(19) United States
(12) Patent Application Publication
(10) Pub. No.: US 2012/0136044 A1
(43) Pub. Date: May 31, 2012

(54) Compositions And Methods Of Inhibiting NADPH Oxidase Expression

(75) Inventors: Hagar Kalinski, Rishon-le-Zion (IL); Igor Mett, Rehovot (IL); Elena Feinstein, Rehovot (IL)

(73) Assignee: Quark Pharmaceuticals, Inc.

(21) Appl. No.: 13/368,064

(22) Filed: Feb. 7, 2012

Related U.S. Application Data
(62) Division of application No. 12/452,123, filed on Jun. 14, 2010.
(60) Provisional application No. 60/934,865, filed on Jun. 15, 2007.

(51) Int. Cl. A61K 31/713; A61P 27/16; A61P 13/12; A61P 17/00; A61P 11/00; A61P 37/06; A61P 25/00; A61P 19/02; C07H 21/02; A61P 27/02

(52) U.S. Cl. 514/44 A; 536/24.5

(57) ABSTRACT

The invention relates to one or more inhibitors, in particular siRNAs, which down-regulate the expression of a NOX gene selected from the group consisting of NOX4, NOX1, NOX2 (gp91phox, CYBB), NOX5, DUOX2, NOXO1, NOXA1 and NOXA2 (p67phox). The invention also relates to a pharmaceutical composition comprising the compound, or a vector capable of expressing the compound, and a pharmaceutically acceptable carrier. The present invention also contemplates a method of treating or preventing the incidence or severity of various diseases or conditions associated with NOX gene comprising administering to the patient the pharmaceutical composition in a therapeutically effective dose so as to thereby treat the patient.

This application claims the benefit of US provisional application No. 60/934,865 filed Jun. 15, 2007, which is hereby incorporated by reference in its entirety.

Throughout this application various patent and scientific publications are cited. The disclosures for these publications in their entireties are hereby incorporated by reference into this application to more fully describe the state of the art to which this invention pertains.

### FIELD OF THE INVENTION

The present invention relates to compounds, pharmaceutical compositions comprising same and methods of use thereof for the inhibition of NADPH oxidase genes, including NOX4, useful in the treatment of diseases and disorders in which expression of Nox4 and other NADPH oxidase genes is adverse. In particular embodiments, the invention provides siRNA oligonucleotides, compositions comprising same and methods of use thereof in the treatment of acute renal failure, hearing loss, acute respiratory distress syndrome and glaucoma.

### BACKGROUND OF THE INVENTION

### sIRNAs and RNA Interference

RNA interference (RNAi) is a phenomenon involving double-stranded (ds) RNA-dependent gene specific posttranscriptional silencing. Originally, attempts to study this phenomenon and to manipulate mammalian cells experimentally were frustrated by an active, non-specific antiviral defense mechanism which was activated in response to long dsRNA molecules (Gil et al. Apoptosis, 2000. 5:107-114). Later it was discovered that synthetic duplexes of 21 nucleotide RNAs could mediate gene specific RNAi in mammalian cells, without the stimulation of the generic antiviral defense mechanisms (see Elbashir et al. Nature 2001, 411:494-498 and Caplen et al. PNAS USA 2001, 98:9742-9747). As a result, small interfering RNAs (siRNAs), which are short double-stranded RNAs, have become powerful tools in attempting to understand gene function. Thus RNA interference (RNAi) refers to the process of sequence-specific post-transcriptional gene silencing in mammals mediated by small interfering RNAs (siRNAs) (Fire et al, Nature 1998. 391, 806) or microRNAs (miRNA; Ambros, Nature 2004 431:7006, 350-55; and Bartel, Cell. 2004. 116(2):281-97). The corresponding process in plants is commonly referred to as specific post transcriptional gene silencing or RNA silencing and is referred to as quelling in fungi.

An siRNA is a double-stranded RNA molecule which inhibits, either partially or fully, the expression of a gene/mRNA of its endogenous or cellular counterpart, or of an exogenous gene such as a viral nucleic acid. The mechanism of RNA interference is detailed infra.

Several studies have revealed that siRNA therapeutics am effective in vivo in both mammals and in humans. Bitko et al., have shown that specific siRNA molecules directed against the respiratory syncytial virus (RSV) nucleocapsid N gene are effective in treating mice when administered intranasally (Bitko et al., Nat. Med. 2005, 11(1):50-55). siRNA has recently been successfully used for inhibition in primates (Tolentino et al., Retina 2004. 24(1):132-138). For a review of the use of siRNA as therapeutics, see Barik (J. Mol. Med. 2005. 83: 764-773). In addition clinical studies with short siRNAs that target the VEGFR1 receptor for the treatment of Age-Related Macular Degeneration (AMD) have been conducted in human patients. In those studies, such siRNA administered by an intravitreal (intraocular) injection was found effective and safe in 14 patients tested (Kaiser, Am. J. Ophthalmol. 2006. 142(4):660-8).

siRNA Structures

The selection and synthesis of siRNA corresponding to known genes has been widely reported; (see for example Ui-Tei et al., J Biomed Biotech. 2006; 2006: 65052; Chalk et al., BBRC. 2004, 319(1): 264-74; Sioud & Leirdal, Met Mol Biol.; 2004, 252:457-69; Levenkova et al., Bioinform. 2004, 20(3):430-2; Ui-Tei et al., NAR. 2004, 32(3):936-48).

For examples of the use of, and production of; modified siRNA see, for example, Braasch et al., Biochem: 2003, 42(26):7967-75; Chiu et al., RNA, 2003, 9(9):1034-48; PCT publications WO 2004/015107 (atugen AG) and WO 02/44321 (Tuschl et al). U.S. Pat. Nos. 5,898,031 and 6,107,094, teach chemically modified oligomers. US Patent Publication Nos. 2005/0080246 and 2005/0042647 relate to oligomeric compounds having an alternating motif and dsRNA compounds having chemically modified internucleoside linkages, respectively.

Other modifications have been disclosed. The inclusion of a 5′-phosphate moiety was shown to enhance activity of siRNAs in Drosophila embryos (Boutla, et al., Curr. Biol. 2001, 11:1776-1780) and is required for siRNA function in human HeLa cells (Schwarz et al., Mol. Cell, 2002, 10:537-48). Amarzguioui et al., (NAR, 2003, 31(2):589-95) showed that siRNA activity depended on the positioning of the 2′-O-methyl modifications. Holen et al (NAR. 2003, 31(9):2401-07) report that an siRNA having small numbers of 2′-O-methyl modified nucleosides gave good activity compared to wild type but that the activity decreased as the numbers of 2′-O-methyl modified nucleosides was increased. Chiu and Rana (RNA. 2003, 9:1034-48) teach that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czaudema et al., NAR. 2003, 31(11):2705-16; WO 2004/015107). The molecules of the present invention offer an advantage in that they are non-toxic and may be formulated as pharmaceutical compositions for treatment of various diseases.

### NADPH Oxidase

The NADPH oxidase (NOX) family of proteins in humans consists of at least thirteen unique gene products: NOX1, NOX2 (gp91phox, CYBB), NOX3, NOX4, NOX5, DUOX1 and DUOX2 and associated proteins p22phox (CYBA), NOXO1, NOXO2 (p47phox, NCF1) NOXA1, NOXA2 (p67phox, NCF2) and p40phox (NCF4) (hereinafter “NOX genes”). Each member of the NOX family has a specific tissue expression pattern. For example, NOX1 is highly expressed in colonic epithelium, NOX2 has a broad expression pattern while NOX4 has been detected primarily in renal tubular epithelium (Geiszt et al., PNAS USA 2000, 97:8010-8014) and proliferating vascular smooth muscle (Lassegue et al., Circ Res. 2001 88(9):888-94). Reactive oxygen species (ROS) generated in many tissues has been shown to originate from the activity of NOX enzymes and NOX gene expression has been associated with various pathological processes (comprehensive review in Bedard and Krause, Physiol. Rev. 2007. 87:245-313, hereby incorporated by reference in its entirety).

International patent publication no. WO 2005/119251 discloses a method of inhibiting NOX3 for treating hearing loss.

International patent publication no. WO 2002/030453 discloses NADPH oxidase inhibitors for reducing angiogenesis.

U.S. Pat. No. 6,846,672 and related patents and patent applications disclose the polynucleotide and polypeptide sequences of the NOX enzymes.

US Patent Publication No. 2007/0037883 relates to NOX4 inhibition.

U.S. Pat. Nos. 6,846,672; 7,029,673; 7,202,052; 7,202,053 and 7,226,769 disclose NOX enzymes and regulators thereof.

International patent publication no. WO 2008/050329 assigned to the assignee of the current invention relates to certain NOX genes.

### Chemical-Induced Toxicity

Certain chemicals, including pharmaceuticals, are known to be toxic to their hosts and a benefit-risk assessment may be warranted. Chemicals such as cisplatin and its analogs (cisplatin-like compounds) and hydroquinone and its analogs, selected antibiotics and loop diuretics are known to be associated with, inter alia, hearing loss (ototoxicity), renal damage (nephrotoxicity) and/or neural damage (neurotoxicity).

The ototoxic effects of various therapeutic drugs on auditory cells and spiral ganglion neurons are often the limiting factor for their therapeutic usefulness. Main ototoxic drugs include the widely used chemotherapeutic agent cisplatin and its analogs, commonly used aminoglycoside antibiotics, e.g. gentamycin, for the treatment of infections caused by gram-negative bacteria, quinine and its analogs, salicylate and its analogs, and loop-diuretics.

For example, antibacterial aminoglycosides such as gentamycins, streptomycins, kanamycin, tobramycins, and the like are known to have serious toxicity, particularly ototoxicity and nephrotoxicity, which reduce the value of such antimicrobials as therapeutic agents (see Goodman and Gihnan's The Pharmacological Basis of Therapeutics, 6th ed, A. Goodman Gilman et al., eds; Macmillan Publishing Co., Inc., New York, 1980, pp. 1169-71).

Chemical induced toxicity especially ototoxicity is a dose-limiting side-effect of antibiotic administration. Chemical induced toxicity, especially ototoxicity, is also a serious dose-limiting side-effect for cisplatin and cisplatin-like compounds, which contain a platinum coordination complex, that have proven effective on a variety of human cancers including testicular, ovarian, bladder, and head and neck cancer. Platinum based drugs include carboplatin, cisplatin, oxaliplatin and satrapaltin inter alia; see Kelland and Farrell, Platinum-based drugs in cancer therapy (Cancer drug discovery & development) Lavoisier 2000 which is hereby incorporated by reference.

Cisplatin (Platinol®) and cisplatin-like compounds (platinum based compounds) damage auditory and vestibular systems.

Salicylates, such as aspirin, are the most commonly used therapeutic drugs for their anti-inflammatory, analgesic, anti-pyretic and anti-thrombotic effects. Unfortunately, they too have ototoxic side effects and can lead to tinnitus (“ringing in the ears”) and temporary hearing loss. Moreover, if the drug is used at high doses for a prolonged time, chronic and irreversible hearing impairment can arise.

Without being bound by theory, it is believed that cisplatin drugs (cisplatin and cisplatin-like compounds) and other potentially ototoxic drugs (such as aminoglycoside antibiotics) may induce the ototoxic effects via programmed cell death or apoptosis in inner ear tissue, particularly inner ear hair cells (Zhang et al., Neuroscience 2003, 120(1):191-205; Wang et al., J. Neuroscience 2003, 23(24):8596-8607). Presbycusis, age related hearing loss, is prevalent in the aging population with about 30-35 percent of adults between the ages of 65 and 75 years and about 40-50 percent of people aged 75 and older affected.

In mammals, auditory hair cells are produced only during embryonic development and do not regenerate if lost during postnatal life, therefore, loss of hair cells results in profound and irreversible deafness. Unfortunately, at present, there are no effective therapies to treat the cochlea and reverse this condition. Thus, an effective therapy to prevent cell death of auditory hair cells would be of great therapeutic value.

Accordingly, there exists a need for means to prevent, reduce or treat the incidence and/or severity of inner ear disorders and hearing impairments involving inner ear tissue, particularly inner ear hair cells and of other diseases or disorders caused by toxicity such as renal damage (nephrotoxicity) and/or neural damage (neurotoxicity).

### Acute Renal Failure

Acute renal failure (ARF) is a clinical syndrome characterized by rapid deterioration of renal function that occurs within days. The principal feature of ARF is an abrupt decline in glomerular filtration rate (GFR), resulting in the retention of nitrogenous wastes (urea, creatinine). Worldwide, severe ARF occurs in about 170-200 per million population annually. To date, there is no specific treatment for established ARF. Several drugs have been found to ameliorate toxic and ischemic experimental ARF, as manifested by lower serum creatinine levels, reduced histological damage and faster recovery of renal function in different animal models. These include anti-oxidants, calcium channel blockers, diuretics, vasoactive substances, growth factors, anti-inflammatory agents and more. However, the drugs tested in clinical trials showed no benefit, and their use in clinical ARF has not been approved.

In the majority of hospitalized ARF patients, ARF is caused by acute tubular necrosis (ATN), which results from ischemic and/or nephrotoxic insults. Renal hypoperfusion is caused by hypovolemic, cardiogenic and septic shock, by administration of vasoconstrictive drugs or renovascular injury. Nephrotoxins include exogenous toxins such as contrast media, aminoglycosides and cisplatin and cisplatin-like compounds as well as endogenous toxin such as myoglobin. Recent studies, however, support the theory that apoptosis in renal tissues is prominent in most human cases of ARF. The principal site of apoptotic cell death is the distal nephron. During the initial phase of ischemic injury, loss of integrity of the actin cytoskeleton leads to flattening of the epithelium, with loss of the brush border, loss of focal cell contacts, and subsequent disengagement of the cell from the underlying substratum. It has been suggested that apoptotic tubule cell death may be more predictive of functional changes than necrotic cell death (Komarov et al., Science 1999, 10; 285(5434):1733-7); Supavekin et al., Kidney Int. 2003, 63(5):1714-24).

In conclusion, currently there are no satisfactory modes of therapy for the prevention and/or treatment of acute renal failure, and there is a need therefore to develop novel compounds for this purpose.

### Glaucoma

Glaucoma is one of the leading causes of blindness in the world. It affects approximately 66.8 million people worldwide and at least 12,000 Americans are blinded by this disease each year (Kahn and Milton, Am J Epidemiol. 1980, 111(6):769-76). Glaucoma is characterized by the degeneration of axons in the optic nerve head, primarily due to elevated intraocular pressure (IOP). One of the most common forms of glaucoma, known as primary open-angle glaucoma (POAG), results from the increased resistance of aqueous humor outflow in the trabecular meshwork (TM), causing IOP elevation and eventual optic nerve damage.

### Acute Respiratory Distress Syndrome

Acute respiratory distress syndrome (ARDS), also known as respiratory distress syndrome (RDS) or adult respiratory distress syndrome (in contrast with infant respiratory distress syndrome, IRDS) is a serious reaction to various forms of injuries to the lung. This is the most important disorder resulting in increased permeability pulmonary edema.

ARDS is a severe lung disease caused by, a variety of direct and indirect insults. It is characterized by inflammation of the lung parenchyma leading to impaired gas exchange with concomitant systemic release of inflammatory mediators causing inflammation, hypoxemia and frequently resulting in multiple organ failure. This condition is life threatening, usually requiring mechanical ventilation and admission to an intensive care unit. A less severe form is called acute lung injury (ALI).

### Spiral Cord Injury

Spinal cord injury or myelopathy, is a disturbance of the spinal cord that results in loss of sensation and/or mobility. The two common types of spinal cord injury are due to trauma and disease. Traumatic injury can be due to automobile accidents, falls, gunshot, diving accidents inter cilia, and diseases which can affect the spinal cord include polio, spina bifida, tumors and Friedreich's ataxia.

### Ischemia Reperfusion Injury Following Lung Transplantation

Lung transplantation, the only definitive therapy for many patients with end stage lung disease, has poor survival rates in all solid allograft recipients. Ischemia reperfusion (IR) injury is one of the leading causes of death in lung allograft recipients.

International patent application no. WO 2006/035434 assigned to the assignee of the present invention discloses p53 inhibitors for the treatment of, inter alia, acute renal failure and hearing loss.

### Oral Mucositis

Oral mucositis, also referred to as a stomatitis, is a common and debilitating side effect of chemotherapy and radiotherapy regimens, which manifests itself as erythema and painful ulcerative lesions of the mouth and throat. Routine activities such as eating, drinking, swallowing, and talking may be difficult or impossible for subjects with severe oral mucositis. Palliative therapy includes administration of analgesics and topical rinses.

### Dry-Eye Syndrome

Dry eye syndrome is a common problem usually resulting from a decrease in the production of tear film that lubricates the eyes. Most patients with dry eye experience discomfort, and no vision loss; although in severe cases, the cornea may become dams or infected. Wetting drops (artificial tears) may be used for treatment while lubricating ointments may help more severe cases.

### Ocular Ischemic Conditions

Ischemic optic neuropathy (ION) includes a variety of disorders that produce ischemia to the optic nerve. By definition, ION is termed anterior if disc edema is present acutely, suggesting infarction of the portion of the optic nerve closest to the globe. ION also may be posterior, lying several centimeters behind the globe. Ischemic optic neuropathy usually occurs only in people older than 60 years of age. Most cases are nonarteritic and attributed to the effects of atherosclerosis, diabetes, or hypertension on optic nerve perfusion. Temporal arteritis causes about 5% of cases (arteritic ION).

Symptoms and signs are sudden, partial or complete vision loss, accompanied by swelling of the optic nerve head and often hemorrhage. Visual field defects may manifest as loss of half the visual field with a horizontal demarcation or as central or centrocecal (surrounding the natural blind spot) scotomata. Decreased vision is soon followed by pallor of the optic disk.

More effective therapies to treat the above mentioned diseases and disorders would be of great therapeutic value.

### SUMMARY OF THE INVENTION

The present invention provides novel double stranded oligonucleotides that inhibit or reduce expression of NOX4, NOX1, NOX2 (gp91phox, CYBB), NOX5, DUOX2, NOXO1, NOXO2 (NCF1), NOXA1 and NOXA2 (p67phox, NCF2 (the “NOX genes”) and pharmaceutical compositions comprising one or more such oligonucleotides or a vector capable of expressing the oligoribonucleotide. The present invention further relates to methods for treating or preventing the incidence or severity of various diseases or conditions in which NOX genes expression is associated with the etiology or progression of the disease or condition.

In one aspect the present invention provides a compound having the structure:

5′(N)ₓ—Z3′ (antisense strand)

3′Z′—(N′)_{y}5′ (sense strand)

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present, or absent, but if present is dTdT and is covalently attached at the 3′ terminus of the strand in which it is present and
wherein the sequence of (N)ₓ comprises an antisense sequence to the mRNA transcribed from a mammalian gene selected from the group consisting of NOX4, NOX1, NOX2 (gp-91phox, CYBB), NOX5, DUOX2, NOXO1, NOXO2 (NCF1), NOXA1 and NOXA2 (p67phox, NCF2).

In certain embodiments the preferred gene is NOX4. The polynucleotide sequence of human NOX4 mRNA (NCBI gene ID no. 50507) is set forth in SEQ ID NO:1, and the corresponding polypeptide is set forth in SEQ ID NO:2.

In some embodiments the compound comprises a phosphodiester bond. In various embodiments the compound comprises ribonucleotides wherein x=y and wherein x is an integer selected from the group consisting of 19 and 21. In preferred embodiments x=y=19.

In some embodiments the compound is blunt ended, for example wherein Z and Z′ are both absent. In an alternative embodiment, the compound comprises at least one 3′ overhang, wherein at least one of Z or Z′ is present.

In some embodiments the compound comprises one or more ribonucleotides unmodified in their sugar residues. In other embodiments the compound comprises at least one ribonucleotide modified in the sugar residue. In some embodiments the compound comprises a modification at the 2′ position of the su residue. Modifications in the 2′ position of the sugar residue include amino, fluoro, methoxy, alkoxy and alkyl moieties. In certain preferred embodiments the modification comprises a ribonucleotide comprising a methoxy moiety at the 2′ position (2′-O-methyl; 2′-O-Me; 2′-O—CH₃) of the sugar residue.

In some embodiments the compound comprises modified alternating ribonucleotides in one or both of the antisense and the sense strands. In preferred embodiments the compound comprises modified alternating ribonucleotides in the antisense and the sense strands. In some preferred embodiments the middle ribonucleotide of the antisense strand is not modified; e.g. ribonucleotide in position 10 in a 19-mer strand.

In additional embodiments the compound comprises modified ribonucleotides in alternating positions wherein the ribonucleotides at the 5′ and 3′ termini of the antisense strand are modified in their sugar residues, and the ribonucleotides at the 5′ and 3′ termini of the sense strand are unmodified in their sugar residues. In some embodiments, neither the antisense nor the sense strands are phosphorylated at the 3′ and 5′ termini. In other embodiments either or both the antisense and the sense strands are phosphorylated at the 3′ termini.

In various embodiments the compound comprises an antisense sequence present in Tables A-R. In other embodiments the present invention provides a mammalian expression vector comprising an antisense sequence present in Tables A-R. In certain preferred embodiments the present invention provides a compound having the structure

5′(N)3′ antisense strand

3′(N′)5′ sense strand

wherein each of N and N′=19 and are fully complementary;
wherein alternating ribonucleotides in the antisense and the sense strands are modified to result in a 2′-O-methyl modification in the sugar residue of the ribonucleotides;
wherein the ribonucleotides at the 5′ and 3′ termini of the antisense strand are modified;
wherein the ribonucleotides at the 5′ and 3′ termini of the sense strand are unmodified;
wherein the antisense and the sense strands are phosphorylated or non-phosphorylated at the 3′ and 5′ termini; and
wherein each of N and N′ is selected from the group of oligomers set forth in Tables A-R.

In a second aspect the present invention provides a pharmaceutical composition comprising one or more compounds of the present invention, in an amount effective to inhibit human NOX gene expression wherein the NOX gene or NOX associated gene is selected from NOX4, NOX1, NOX2 (gp91phox, CYBB), NOX5, DUOX2, NOXO1, NOXO2, NOXA1 and NOXA2 (p67phox); and a pharmaceutically acceptable carrier.

In another aspect, the present invention relates to a method for the treatment of a subject in need of treatment for a disease or disorder associated with the expression of a NOX gene wherein the NOX gene is selected from NOX4, NOX1, NOX2 (gp91phox, CYBB), NOX5, DUOX2, NOXO1, NOXO2, NOXA1 and NOXA2 (p67phox), comprising administering to the subject an amount of an siRNA which reduces or inhibits expression of at least one of the NOX genes.

More specifically, the present invention provides methods and compositions useful in treating a patient suffering from acute renal failure (ARF), hearing loss, ocular disease (e.g. glaucoma), acute respiratory distress syndrome (ARDS) and other acute lung and respiratory injuries, lung transplantation, nephrotoxicity, spinal cord injury, pressure sores, and chronic obstructive pulmonary disease (COPD).

The methods of the invention comprise administering to the patient one or more inhibitory compounds which down-regulate expression of a NOX gene (e.g. NOX4) and in particular siRNA in a therapeutically effective dose so as to thereby treat the patient.

In various embodiments the inhibitor is selected from the group consisting of an siRNA, shRNA, an aptamer, an antisense molecule, miRNA, a ribozyme, and an antibody. In preferred embodiments the inhibitor is siRNA.

According to one embodiment, the present invention provides improved a method for treating or preventing hearing loss associated with an ototoxic drug comprising administering to a subject in need thereof an ototoxic drug having a hearing-impairing side-effect, in combination with a therapeutically effective amount of at least one siRNA compound that inhibits expression of a NOX gene The compositions of the invention can be administered at a suitable interval(s) either prior to, subsequent to, or substantially concurrent with the administration of the ototoxic, hearing-impairing drug that induces inner ear apoptotic tissue damage. In some embodiments, the siRNA and the ototoxic drug are administered separately. In some embodiments the siRNA molecules that inhibit a NOX gene are administered locally while the ototoxic, hearing-impairing drug is administered systemically. The siRNA molecules may be administered prior to, simultaneously with or subsequent to the ototoxic drug.

In some embodiments the present invention provides a pharmaceutical composition comprising an siRNA that inhibits expression of a NOX gene; and a pharmaceutically acceptable carrier.

In certain embodiments the pharmaceutical composition further comprises an ototoxic drug. The pharmaceutical composition has lower ototoxicity than the ototoxic pharmaceutical alone, and, preferably, has a higher dosage of the ototoxic pharmaceutical than typically used Examples of such improved compositions include cisplatin or other ototoxic neoplastic agent or an aminoglycoside antibiotic(s) in combination with the therapeutically effective amount of one or more siRNA molecules that inhibit a NOX gene.

Still further, the invention relates to the use of the compositions of the invention in cases where diuretics are needed. The present invention provides a solution to the art that has long sought a therapy and a medicament which can treat the ototoxic effects associated with certain diuretics, and particular with the more popular and commonly used loop-diuretics, without sacrificing their diuretic effectiveness.

In yet other embodiments, the invention relates to the use of the compositions in subjects requiring quinine or quinine-like compounds. The present invention provides a solution to the art that has long sought a therapy and a medicament which can treat the ototoxic effects currently associated with certain quinines without sacrificing their effectiveness.

The present invention further relates to the use of compounds which down-regulate the expression of a NOX gene, particularly to small interfering RNAs (siRNAs), in the treatment of various diseases, conditions or disorders associated with a NOX gene expression including hearing loss, acute renal failure, ocular disease (e.g. glaucoma), Acute Respiratory Distress Syndrome (ARDS) and other acute lung injuries, lung transplantation, spinal cord injury, pressure sores, and Chronic Obstructive Pulmonary Disease (COPD).

Lists of 19- and 21-mer sense and corresponding antisense sequences useful in preparation of siRNA compounds are set forth in Tables A-R.

A list of preferred siRNA to NOX4 is provided in tables A-B, infra.

A list of preferred siRNA to NOX1 is provided in tables C-D, infra.

A list of preferred siRNA to NOX2 (CYBB) is provided in tables E-F, infra.

A list of preferred siRNA to NOX5 is provided in tables G-H, infra.

A list of preferred siRNA to DUOX2 is provided in tables infra.

A list of preferred siRNA to NOXO1 is provided in tables K-L, infra.

A list of preferred siRNA to NOXO2 (NCF1) is provided in tables M-N, infra.

A list of preferred siRNA to NOXA1 is provided in tables O—P, infra.

A list of preferred siRNA to NOXA2 (p67phox, NCF2) is provided in tables Q-R, infra.

All sequences are provided in 5′-3′ orientation. Where cross-species specificity is known, the species is listed as well. In certain embodiments the preferred gene is NOX4. Preferred siRNA compounds are blunt ended 19 base pair compounds, which comprise an oligomer pair set forth in Table A.

These and further features of the present invention will be better understood in conjunction with the detailed description, examples and claims that follow.

### DETAILED DESCRIPTION OF THE INVENTION

The present invention relates generally to compounds which down-regulate expression of NOX genes, particularly to novel small interfering RNAs (siRNAs), and to the use of these siRNAs in the treatment of various diseases and medical conditions. Particular diseases and conditions to be treated are hearing loss including chemical-induced oxotoxicity; acute renal failure (ARF), nephritis, ocular disease (e.g glaucoma), Acute Respiratory Distress Syndrome (ARDS) and other acute lung injuries, lung transplantation, nephrotoxicity, spinal cord injury, pressure sores, osteoarthritis (OA) and Chronic Obstructive Pulmonary Disease (COPD).

Lists of preferred siRNA are provided in Tables A-R, infra. There are separate lists of 19-mer and 21-mer siRNAs, which are prioritized based on their score according to a proprietary algorithm as the best sequences for targeting the human gene expression. Methods, molecules and compositions, which inhibit the NOX genes are discussed herein at length, and any of said molecules and/or compositions may be beneficially employed in the treatment of a patient suffering from any of said conditions. Tables A, C, E, G, I, K, M, O, Q set forth 19-mer oligomers. Tables B, D, F, H, J, L, N, P, R set forth 21-mer oligomers.

### DEFINITIONS

For convenience certain terms employed in the specification, examples and claims are described herein.

It is to be noted that, as used herein, the singular forms “a”, “an” and “the” include plural forms unless the content clearly dictates otherwise.

Where aspects or embodiments of the invention are described in terms of Markush groups or other grouping of alternatives, those skilled in the art will recognize that the invention is also thereby described in terms of any individual member or subgroup of members of the group.

An “inhibitor” is a compound which is capable of reducing the expression of a gene or the activity of the product of such gene to an extent sufficient to achieve a desired biological or physiological effect. The term “inhibitor” as used herein refers to one or more of an oligonucleotide inhibitor, including siRNA, shRNA, [synthetic shRNA; miRNA, antisense RNA and DNA and ribozymes. Inhibition may also be referred to as down-regulation or, for RNAi, silencing.

The term “inhibit” as used herein refers to reducing the expression of a gene or the activity of the product of such gene to an extent sufficient to achieve a desired biological or physiological effect Inhibition may be complete or partial.

As used herein, the term “inhibition” of a NOX gene means inhibition of the gene expression (transcription or translation) or polypeptide activity of a gene selected from the group NOX4, NOX1, NOX2 (gp91phox, CYBB), NOX5, DUOX2, NOXO1, NOXO2, NOXA1 and NOXA2 (p67phox), or SNP (single nucleotide polymorphism) or other variants thereof.

As used herein, the terms “polynucleotide” and “nucleic acid” may be used interchangeably and refer to nucleotide sequences comprising deoxyribonucleic acid (DNA), and ribonucleic acid (RNA). The terms should also be understood to include, as equivalents, analogs of either RNA or DNA made from nucleotide analogs. Throughout this application mRNA sequences are set forth as representing the corresponding genes. The terms “mRNA polynucleotide sequence” and mRNA are used interchangeably.

“Oligonucleotide” or “oligomer” refers to a deoxyribonucleotide or ribonucleotide sequence from about 2 to about 50 nucleotides. Each DNA or RNA nucleotide may be independently natural or synthetic, and or modified or unmodified. Modifications include changes to the sugar moiety, the base moiety and or the linkages between nucleotides in the oligonucleotide. The compounds of the present invention encompass molecules comprising deoxyribonucleotides, ribonucleotides, modified deoxyribonucleotides, modified ribonucleotides and combinations thereof.

“Nucleotide” is meant to encompass deoxyribonucleotides and ribonucleotides, which may be natural or synthetic, and or modified or unmodified. Modifications include changes and substitutions to the sugar moiety, the base moiety and/or the internucleotide linkages.

All analogs of, or modifications to, a nucleotide/oligonucleotide may be employed with the present invention, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

As used herein, the term “polypeptide” refers to, in addition to a polypeptide, an oligopeptide, peptide and a full protein. For example, several human NOXO1 and NOX1 isoforms are known to be expressed. The present invention provides molecules that inhibit one or more of the isoforms.

RNA Interference and siRNA

RNA interference (RNAi) is based on the ability of dsRNA species to enter a cytoplasmic protein complex, where it is then targeted to the complementary cellular RNA and specifically degrade it. The RNA interference response features an endonuclease complex containing an siRNA, commonly referred to as an RNA-induced silencing complex (RISC), which mediates cleavage of single-stranded RNA having a sequence complementary to the antisense strand of the siRNA duplex. Cleavage of the target RNA may take place in the middle of the region complementary to the antisense strand of the siRNA duplex (Elbashir et al., Genes Dev., 2001, 15(2):188-200). In more detail, longer dsRNAs are digested into short (17-29 bp) dsRNA fragments (also referred to as short inhibitory RNAs, “siRNAs”) by type III RNAses (DICER, DROSHA, etc.; Bernstein et al., Nature, 2001, 409(6818):363-6; Lee et al., Nature, 2003, 425(6956):415-9). The RISC protein complex recognizes these fragments and complementary mRNA. The whole process is culminated by endonuclease cleavage of target mRNA (McManus & Sharp, Nature Rev Genet, 2002, 3(10):737-47; Paddison & Hannon, Curr Opin Mol Ther. 2003, 5(3):217-24). (For additional information on these terms and proposed mechanisms, see for example Bernstein et al., RNA 2001, 7(11):1509-21; Nishikura, Cell 2001, 107(4):415-8 and PCT publication WO 01/36646).

The selection and synthesis of siRNA corresponding to known genes has been widely reported; see for example Ui-Tei et al., J Biomed Biotechnol. 2006; 2006: 65052; Chalk et al., Biochem. Biophys. Res. Comm. 2004, 319(1): 264-74; Sioud & Leirdal, Met. Mol Biol.; 2004, 252:457-69; Levenkova et al., Bioinform. 2004, 20(3):430-2; Ui-Tei et al., Nuc. Acid Res. 2004, 32(3):936-48. For examples of the use of, and production of, modified siRNA see Braasch et al., Biochem., 2003, 42(26):7967-75; Chiu et al., RNA, 2003, 9(9):1034-48; PCT publications WO 2004/015107 (Atugen); WO 02/44321 (Tuschl et al), and U.S. Pat. Nos. 5,898,031 and 6,107,094. See also Dykxhoorn et al 2006, 13, 541-552.

Several groups have described the development of DNA-based vectors capable of generating siRNA within cells. The method generally involves transcription of short hairpin RNAs that are efficiently processed to form siRNAs within cells (Paddison et al. PNAS USA 2002, 99:1443-1448; Paddison et al. Genes & Dev 2002, 16:948-958; Sui et al. PNAS USA 2002, 8:5515-5520; and Brummelkamp et al. Science 2002, 296:550-553). These reports describe methods to generate siRNAs capable of specifically targeting numerous endogenously and exogenously expressed genes.

As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, double-stranded polynucleotides and single-stranded polynucleotides such as sense or antisense.

“Oligonucleotide” refers to a sequence having from about 2 to about 50 linked nucleotides or linked modified nucleotides, or a combination of modified and unmodified nucleotide. Oligonucleotide includes the terms oligomer, antisense strand and sense strand.

“Nucleotide” is meant to encompass deoxyribonucleotides and ribonucleotides, which may be natural or synthetic, and or modified or unmodified. Modifications include changes to the sugar moiety, the base moiety and or the linkages between ribonucleotides in the oligoribonucleotide. Table 1, below, sets forth the gene identification number (gi) with an NCBI accession number and the corresponding oligomer tables' listings.

TABLE 1

| | | | Preferred indications or |
|---|---|---|---|
| | | | diseases to be treated |
| | | Oligomer | according to the present |
| | Full name and | tables (for | invention (particularly |
| | Human Gene | siRNA | preferred indications are |
| Gene | ID (mRNA) | compounds) | underlined) |
| NOX4 | NADPH oxidase 4 | A, B | ARF, hearing loss, |
| | (gi:20149638, | | ARDS, glaucoma, |
| | NM_016931, | | spinal cord injury, |
| | SEQ ID NO: 1) | | COPD, DR, nephritis |
| NOX1 | NADPH oxidase 1 | C, D | ARF, hearing loss, |
| | (gi:21614529, | | ARDS, glaucoma, |
| | NM_007052 | | spinal cord injury, |
| | isoform 1L; | | COPD, DR, nephritis |
| | SEQ ID NO: 2) | | |
| | (gi:7669509, | | |
| | NM_013955 | | |
| | isoform 1Lv; | | |
| | SEQ ID NO: 3) | | |
| NOX2 | NADPH oxidase 2 | E, F | ARF, hearing loss, |
| (gp91pho, | (gi:6996020, | | ARDS, glaucoma, |
| CYBB) | NM_000397; | | spinal cord injury, |
| | SEQ ID NO: 4) | | COPD, OA, DR, |
| | | | nephritis |
| NOX5 | NADPH oxidase 5 | G, H | ARF, hearing loss, |
| | (gi:20127623, | | ARDS, glaucoma, |
| | NM_024505; | | spinal cord injury, |
| | SEQ ID NO: 5) | | COPD, DR, nephritis |
| DUOX2 | Dual oxidase 2 | I, J | ARF, hearing loss, |
| | (gi:132566531, | | ARDS, glaucoma, |
| | NM_014080; | | spinal cord injury, |
| | SEQ ID NO: 6) | | COPD, DR, nephritis |
| NOXO1 | NADPH oxidase | K, L | ARF, hearing loss, |
| | organizer 1 | | ARDS, glaucoma, |
| | (gi:34222190, | | spinal cord injury, |
| | variant a, | | COPD, DR, nephritis |
| | NM_144603, | | |
| | SEQ ID NO: 7) | | |
| | (gi:41281810, | | |
| | variant b, | | |
| | NM_172167; | | |
| | SEQ ID NO: 8) | | |
| | (gi:41281827, | | |
| | variant c, | | |
| | NM_172168; | | |
| | SEQ ID NO: 9) | | |
| NOXO2 | NADPH oxidase | M, N | ARF, hearing loss, |
| (p47phox, | organizer 2 | | ARDS, glaucoma, |
| NCF1) | (gi:115298671, | | spinal cord injury, |
| | NM_000265; | | COPD, DR, nephritis |
| | SEQ ID NO: 10) | | |
| NOXA1 | NADPH oxidase | O, P | ARF, hearing loss, |
| | activator 1 | | ARDS, glaucoma, |
| | (gi:41393186, | | spinal cord injury, |
| | NM_006647; | | COPD, DR, nephritis |
| | SEQ ID NO: 11) | | |
| NOXA2 | NADPH oxidase | Q, R | ARF, hearing loss, |
| (p67phox, | activator 2 | | ARDS, glaucoma, |
| NCF2) | (gi:67189969, | | spinal cord injury, |
| | NM_000433; | | COPD, DR, nephritis |
| | SEQ ID NO: 12) | | |

ARF: acute renal failure,
ARDS, acute respiratory distress syndrome,
COPD chronic obstructive pulmonary disease,
DR: diabetic retinopathy.

All analogues of, or modifications to, a nucleotide/oligonucleotide may be employed with the present invention, provided that said analogue or modification does not substantially affect the function of the nucleotide/oligonucleotide. The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, psuedo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogues of polynucleotides can be prepared wherein the structure of one or more nucleotide is fundamentally altered and better suited as therapeutic or experimental reagents. An example of a nucleotide analogue is a peptide nucleic acid (PNA) wherein the deoxyribose (or ribose) phosphate backbone in DNA (or RNA is replaced with a polyamide backbone which is similar to that found in peptides. PNA analogues have been shown to be resistant to enzymatic degradation and to have extended lives in vivo and in vitro. Other modifications include locked nucleic acids (LNA), morpholinos, mirror nucleotides (L-nucleotide; spieglemer), polymer backbones, cyclic backbones, or acyclic backbones. Examples of siRNA compounds comprising LNA nucleotides are disclosed in Ehnen et al., (NAR 2005. 33(1):439-447).

A “mirror” nucleotide is a nucleotide with reversed chirality to the naturally occurring or commonly employed nucleotide, i.e., a mirror image (L-nucleotide) of the naturally occurring (D-nucleotide). The nucleotide can be a ribonucleotide or a deoxyribonucleotide and my further comprise at least one sugar, base and or backbone modification. U.S. Pat. No. 6,602,858 discloses nucleic acid catalysts comprising at least one L-nucleotides substitution.

The present invention provides methods and compositions for inhibiting expression of a NOX gene in vivo. In general, the method includes administering oligoribonucleotides, in particular small interfering RNAs (i.e., siRNAs) or a nucleic acid material that can produce siRNA in a cell, that targets an mRNA transcribed from a NOX gene in an amount sufficient to down-regulate expression of a target gene by an RNA interference mechanism. In particular, the subject method can be used to inhibit expression of NOX4 for treatment of a disease.

In accordance with the present invention, the siRNA molecules or other inhibitors of the NOX genes are used as drugs to treat various pathologies.

The present invention provides double-stranded oligoribonucleotides (eg. siRNAs), which down-regulate the expression of the NOX genes selected from the group consisting of NOX4, NOX1, NOX2 (gp91phox, CYBB), NOX5, DUOX2, NOXO1, NOXO2, NOXA1 and NOXA2 (p67phox). An siRNA of the invention is a duplex oligoribonucleotide in which the sense strand is derived from the mRNA sequence of the NOX genes, and the antisense strand is complementary to the sense strand. In general, some deviation from the target mRNA sequence is tolerated without compromising the siRNA activity (see e.g. Czauderna et al., Nuc. Acids Res. 2003, 31(11):2705-2716). An siRNA of the invention inhibits gene expression on a post-transcriptional level with or without destroying the mRNA. Without being bound by theory, siRNA may target the mRNA for specific cleavage and degradation and/or may inhibit translation from the targeted message.

In some embodiments the oligoribonucleotide according to the present invention comprises modified siRNA. In various embodiments the siRNA comprises an RNA duplex comprising a first strand and a second strand, whereby the first strand comprises a ribonucleotide sequence at least partially complementary to about 18 to about 40 consecutive nucleotides of a target nucleic acid, and the second strand comprises ribonucleotide sequence at least partially complementary to the first strand and wherein said first strand and/or said second strand comprises a plurality of groups of modified ribonucleotides having a modification at the 2′-position of the sugar moiety whereby within each strand each group of modified ribonucleotides is flanked on one or both sides by a group of flanking ribonucleotides whereby each ribonucleotide forming the group of flanking ribonucleotides is selected from an unmodified ribonucleotide or a ribonucleotide having a modification different from the modification of the groups of modified ribonucleotides. The oligoribonucleotide according to the present invention may comprise a 2′ modification on the sugar moiety of at least one ribonucleotide (“2′ sugar modification”). In certain embodiments the compound comprises 2′O-alkyl or 2′-fluoro or 2′O-allyl or any other 2′ sugar modification, optionally on alternate positions.

Other stabilizing modifications are also possible (eg. modified nucleotides added to a 3′ or 5′ terminus of an oligomer). Other modifications include additions to the 5′ and/or 3′termini of the oligonucleotides. Such terminal modifications may be lipids, peptides, sugars or other molecules.

The group of modified ribonucleotides and/or the group of flanking ribonucleotides comprise a number of ribonucleotides selected from the group consisting of an integer from 1 to 10. Accordingly, the group thus comprises one nucleotide, two nucleotides, three nucleotides, four nucleotides, five nucleotides, six nucleotides, seven nucleotides, eight nucleotides, nine nucleotides or ten nucleotides.

The groups of modified nucleotides and flanking nucleotides may be organized in a pattern on at least one of the strands.

In some embodiments the first and second strands comprise a pattern of modified nucleotides. In various embodiments the pattern of modified nucleotides of said first strand is identical relative to the pattern of modified nucleotides of the second strand.

In other embodiments the pattern of modified nucleotides of said first strand is shifted by one or more nucleotides relative to the pattern of modified nucleotides of the second strand.

In some preferred embodiments the middle ribonucleotide in the first strand (antisense) is an unmodified nucleotide. For example, in a 19-oligomer antisense strand, ribonucleotide number 10 is unmodified; in a 21-oligomer antisense strand, ribonucleotide number 11 is unmodified; and in a 23-oligomer antisense strand, ribonucleotide number 12 is unmodified. The modifications or pattern of modification, if any, of the siRNA must be planned to allow for this.

The modifications on the 2′ moiety of the sugar residue include amino, fluoro, methoxy alkoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O-, S-, or N-alkyl; O-, S, or N-alkenyl; SOCH₃; SO₂CH₃; ONO₂; NO₂, N₃; heterozycloalkyl; heterozycloalkaryl; aminoalkylamino; polyalkylamino or substituted silyl, as described, inter alia, in European patents EP 0 586 520 B1 and EP 0 618 925 B1.

In some embodiments the siRNA is blunt ended, on one or both ends. More specifically, the siRNA may be blunt ended on the end defined by the 5′-terminus of the first strand and the 3′-terminus of the second strand, or the end defined by the 3′-terminus of the first strand and the 5′-terminus of the second strand.

In other embodiments at least one of the two strands may have an overhang of at least one nucleotide at the 5′-terminus; the overhang may consist of at least one deoxyribonucleotide. At least one of the strands may also optionally have an overhang of at least one nucleotide at the 3′-terminus. The overhang may consist of from about 1 to about 4 nucleotides.

The length of RNA duplex is from about 18 to about 40 ribonucleotides, preferably 19 to 23 ribonucleotides. Further, the length of each strand (oligomer) may independently have a length selected from the group consisting of about 15 to about 40 bases, preferably 18 to 23 bases and more preferably 19 or 21 ribonucleotides.

Additionally, in certain preferred embodiments the complementarity between said first strand and the target nucleic acid can be perfect. In some embodiments, the strands are substantially complementary, i.e. having one, two or up to three mismatches between said first strand and the target nucleic acid.

In some embodiments the first strand and the second strand each comprise at least one group of modified ribonucleotides and at least one group of flanking ribonucleotides, whereby each group of modified ribonucleotides comprises at least one ribonucleotide and whereby each group of flanking ribonucleotides comprises at least one ribonucleotide, wherein each group of modified ribonucleotides of the first strand is aligned with a group of flanking ribonucleotides on the second strand, and wherein the 5′ most terminal ribonucleotide is selected from a group of modified ribonucleotides, and the 3′ most terminal ribonucleotide of the second strand is a selected from the group of flanking ribonucleotide. In some embodiments each group of modified ribonucleotides consists of a single ribonucleotide and each group of flanking ribonucleotides consists of a single nucleotide.

In yet other embodiments the ribonucleotide forming the group of flanking ribonucleotides on the first strand is an unmodified ribonucleotide arranged in a 3′ direction relative to the ribonucleotide forming the group of modified ribonucleotides, and the ribonucleotide forming the group of modified ribonucleotides on the second strand is a modified ribonucleotide which is arranged in 5′ direction relative to the ribonucleotide forming the group of flanking ribonucleotides. In some embodiments the first strand of the siRNA comprises five to about twenty, eight to twelve, preferably ten or twelve groups of modified ribonucleotides, and the second strand comprises seven to eleven, preferably nine or eleven groups of modified ribonucleotides. The first strand and the second strand may be linked by a loop structure, which may be comprised of a non-nucleic acid polymer such as, inter alia, polyethylene glycol. Alternatively, the loop structure may be comprised of a nucleic acid, including modified and non-modified ribonucleotides and modified and non-modified deoxyribonucleotides.

Further, the 5′-terminus of the first strand of the siRNA may be linked to the 3′-terminus of the second strand, or the 3′-terminus of the first strand may be linked to the 5′-terminus of the second strand, said linkage being via a nucleic acid linker typically having a length between 3-100 nucleotides, preferably about 3 to about 10 nucleotides.

According to the present invention the siRNA compounds are chemically and or structurally modified according to the following, or as tandem siRNA or RNAstar.

In one aspect the present invention provides a compound set forth as Structure (A):

5′(N)ₓ—Z3′ (antisense strand)

3′Z′—(N′)_{y}5′ (sense strand) (A)

wherein each of N and N′ is a nucleotide selected from an unmodified ribonucleotide, a modified ribonucleotide, an unmodified deoxyribonucleotide and a modified deoxyribonucleotide;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present; and
wherein the sequence of (N)ₓ comprises an antisense sequence having substantial identity to about 18 to about 40 consecutive ribonucleotides in the mRNA transcribed from a gene selected from the group consisting of NOX4, NOX1, NOX2 (gp91phox, CYBB), NOX5, DUOX2, NOXO1, NOXA1 and NOXA2 (p67phox), with the proviso that each of (N)x and (N′)y does not comprise more than 50%, 60%, 70% or 80% unmodified deoxyribonucleotide or modified deoxyribonucleotide. In certain embodiments the antisense and sense sequences are selected from the sequences presented in Tables A-R. In preferred embodiments the antisense and sense sequences are selected from Table A.

It will be readily understood by those skilled in the art that the compounds of the present invention consist of a plurality of modified and/or unmodified ribonucleotides, which are linked through covalent linkages. Each such covalent linkage may be a phosphodiester linkage, a phosphorothioate linkage, or a combination of both, along the length of the ribonucleotide sequence of the individual strand. Other possible backbone modifications are described inter alia in U.S. Pat. Nos. 5,587,361; 6,242,589; 6,277,967; 6,326,358; 5,399,676; 5,489,677; and 5,596,086.

In particular embodiments, x and y are independently an integer between about 18 to about 40, preferably from about 19 to about 23. In a particular embodiment, x is equal to y (i.e. x=y) and in preferred embodiments x=y=19, x=y=20 or x=y=21. In a particularly preferred embodiment x=y=19.

In one embodiment of the compound of the invention, Z and Z′ are both absent; in another embodiment one of Z or Z′ is present.

In one embodiment all of the ribonucleotides of the compound are unmodified in their sugar residues.

In certain preferred embodiments at least one ribonucleotide is modified in its sugar residue, preferably by the addition of a moiety at the 2′ position. A preferred moiety is selected from the group consisting of amino, fluoro, methoxy, alkoxy and alkyl groups. In a presently preferred embodiment the moiety at the 2′ position is methoxy (2′-O-Me).

Thus, in certain embodiments the present invention provides a compound having structure B (structures having alternating 2′-O-methyl modification in both strands:

5′(N)x3′ antisense strand

3′(N′)y5′ sense strand (B)

wherein each of (N)ₓ and (N′)_{y} is an oligomer in which each consecutive N or N′ is an unmodified ribonucleotide or a modified ribonucleotide joined to the next N or N′ by a covalent bond;
wherein each of x and y=19, 21 or 23 and (N)ₓ and (N′)_{y} are fully complementary
wherein alternating ribonucleotides in each of (N)ₓ and (N′)_{y} are modified to result in a 2′-O-methyl modification in the sugar residue of the ribonucleotides;
wherein the sequence of (N′)_{y} is a sequence complementary to (N)ₓ; and wherein the sequence of (N)ₓ is present in any one of Tables A-R.

In some embodiments each of (N)ₓ and (N′)_{y} is independently phosphorylated or non-phosphorylated at the 3′ and 5′ termini.

In certain embodiments of the invention, alternating ribonucleotides are modified in both the antisense and the sense strands of the compound.

In certain embodiments wherein each of x and y=19 or 23, each N at the 5′ and 3′ termini of (N)ₓ is modified; and

each N′ at the 5′ and 3′ termini of (N′)_{y} is unmodified.

In certain embodiments wherein each of x and y=21, each N at the 5′ and 3′ termini of (N)ₓ is unmodified; and

each N′ at the 5′ and 3′ termini of (N′)_{y} is modified.

In particular embodiments, when x and y=19, the siRNA is modified such that a 2′-O-methyl (2′-OMe) group is present on the first, third, fifth, seventh, ninth, eleventh, thirteenth, fifteenth, seventeenth and nineteenth nucleotide of the antisense strand (N)ₓ, and whereby the very same modification, i.e. a 2′-OMe group, is present at the second, fourth, sixth, eighth, tenth, twelfth, fourteenth, sixteenth and eighteenth nucleotide of the sense strand (N′)_{y}. In various embodiments these particular siRNA compounds are blunt ended at both termini.

In preferred embodiments of the invention, alternating ribonucleotides are modified in both the antisense and the sense strands of the compound. In particular the exemplified siRNA has been modified such that a 2′-O-methyl (Me) group was present on the first, third, fifth, seventh, ninth, eleventh, thirteenth, fifteenth, seventeenth and nineteenth nucleotide of the antisense strand, whereby the very same modification, i.e. a 2′-O-Me group, was present at the second, fourth, sixth, eighth, tenth, twelfth, fourteenth, sixteenth and eighteenth nucleotide of the sense strand. Additionally, it is to be noted that these particular siRNA compounds are also blunt ended.

In various preferred embodiments of the compounds of the invention having alternating ribonucleotides modified in both the antisense and the sense strands of the compound, for 19-mers and 23-mers the ribonucleotides at the 5′ and 3′ termini of the antisense strand are modified in their sugar residues, and the ribonucleotides at the 5′ and 3′ termini of the sense strand are unmodified in their sugar residues. For 21 mere the ribonucleotides at the 5′ and 3′ termini of the sense strand are modified in their sugar residues, and the ribonucleotides at the 5′ and 3′ termini of the antisense strand are unmodified in their sugar residues. As mentioned above, it is preferred that the middle nucleotide of the antisense strand is unmodified.

According to one preferred embodiment of the invention, the antisense and the sense strands of the siRNA are phosphorylated only at the 3′-terminus and not at the 5′-terminus. According to another preferred embodiment of the invention, the antisense and the sense strands are non-phosphorylated. According to yet another preferred embodiment of the invention, the 5′ most ribonucleotide in the sense strand is modified, for example to abolish any possibility of in vivo 5′-phosphorylation.

The invention further provides a vector capable of expressing any of the aforementioned oligoribonucleotides in unmodified form in a cell after which appropriate modification may be made. In preferred embodiment the cell is a mammalian cell, preferably a human cell.

Substantially complementary refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

More particularly, the invention provides an antisense oligoribonucleotide wherein one strand comprises consecutive nucleotides having, from 5′ to 3′, the sequence set forth in Tables A-R, or a homolog thereof wherein in up to two of the ribonucleotides in each terminal region is altered.

The terminal region of the oligoribonucleotide refers to bases 1-4 and/or 16-19 in the 19-mer sequence and to bases 1-4 and/or 18-21 in the 21-mer sequence.

Additionally, the invention provides sense oligoribonucleotides wherein one strand comprises consecutive nucleotides having, from 5′ to 3′, the sequence set forth in Tables A-R or a homolog thereof wherein in up to two of the ribonucleotides in each terminal region is altered.

Preferred lists of siRNA (sense and antisense strands) directed to NOX4 are set forth in Tables A and B. Tables C through R provide lists of 19- and 21-mer sequences useful in the preparation of siRNA compounds NOX1, NOX2 (gp91phox, CYBB), NOX5, DUOX2, NOXO1, NOXO2, NOXA1 and NOXA2 (p67phox), respectively. The presently most preferred compound of the invention is a blunt-ended 19-mer siRNA, i.e. x=y=19 and Z and Z′ are both absent. The siRNA is either phosphorylated at 3′ termini of both sense and anti-sense strands, or non-phosphorylated at all; or having the 5′ most ribonucleotide on the sense strand specifically modified to abolish any possibility of in vivo 5′-phosphorylation. The alternating ribonucleotides are modified at the 2′ position of the sugar residue in both the antisense and the sense strands, wherein the moiety at the 2′ position is methoxy (2′-O-methyl) and wherein the ribonucleotides at the 5′ and 3′ termini of the antisense strand are modified in their sugar residues, and the ribonucleotides at the 5′ and 3′ termini of the sense strand are unmodified in their sugar residues.

For all the above structures, in various embodiments x=y and each of x and y is 19, 20, 21, 22 or 23. In certain embodiments, x=y=19. In yet other embodiments x=y=23. In additional embodiments the compound comprises modified ribonucleotides in alternating positions wherein each N at the 5′ and 3′ termini of (N)x are modified in their sugar residues and the middle ribonucleotide is not modified, e.g. ribonucleotide in position 10 in a 19-mer strand or 11 in a 21-mer strand or 12 in a 23-mer strand.

In some embodiments, neither (N)x nor (N′)y are phosphorylated at the 3′ and 5′ termini. In other embodiments either or both (N)x and (N′)y are phosphorylated at the 3′ termini. In yet another embodiment, either or both (N)x and (N′)y are phosphorylated at the 3′ termini using non-cleavable phosphate groups. In yet another embodiment, either or both (N)x and (N′)y are phosphorylated at the terminal 2′ termini position using cleavable or non-cleavable phosphate groups.

In certain embodiments for all the above-mentioned Structures, the compound is blunt ended, for example wherein both Z and Z′ are absent. In an alternative embodiment, the compound comprises at least one 3′ overhang, wherein at least one of Z or Z′ is present. Z and Z′ can be independently comprise one or more covalently linked modified or non-modified nucleotides. In some embodiments each of Z and Z′ are independently selected from dT and dTdT. siRNA in which Z and/or Z′ is present have similar activity and stability as siRNA in which Z and Z′ are absent.

Additional nucleic acids according to the present invention comprise at least 14 contiguous nucleotides of any one of the polynucleotides in Tables A-R and more preferably 14 contiguous nucleotide base pairs at any end of the double-stranded structure. It will be understood by one skilled in the art that given the potential length of the nucleic acid according to the present invention and particularly of the individual stretches forming such nucleic acid according to the present invention, some shifts relative to the coding sequence of the mammalian NOX genes of the present invention to each side is possible, whereby such shifts can be up to 1, 2, 3, 4, 5 and 6 nucleotides in both directions, and whereby the thus generated double-stranded nucleic acid molecules shall also be within the present invention.

An additional novel molecule provided by the present invention is an oligonucleotide comprising consecutive nucleotides wherein a first segment of such nucleotides encode a first inhibitory RNA molecule, a second segment of such nucleotides encode a second inhibitory RNA molecule, and a third segment of such nucleotides encode a third inhibitory RNA molecule. Each of the first, the second and the third segment may comprise one strand of a double stranded RNA and the first, second and third segments may be joined together by a linker. Further, the oligonucleotide may comprise three double stranded segments joined together by one or more linker.

Thus, one molecule provided by the present invention is an oligonucleotide comprising consecutive nucleotides which encode three inhibitory RNA molecules; said oligonucleotide may possess a triple stranded structure, such that three double stranded arms are linked together by one or more linker, such as any of the linkers presented hereinabove. This molecule forms a “star”-like structure, and may also be referred to herein as RNAstar. Said triple-stranded oligonucleotide may be an oligoribonucleotide having the general structure:

| | | | | |
|---|---|---|---|---|
| 5′ | Oligo1 (sense) | LINKER A | Oligo2 (sense) | 3′ |
| 3′ | Oligo1 (antisense) | LINKER B | Oligo3 (sense) | 5′ |
| 3′ | Oligo3 (antisense) | LINKER C | Oligo2 (antisense) | 5′ |
| or | | | | |
| 5′ | Oligo1 (sense) | LINKER A | Oligo2 (antisense) | 3′ |
| 3′ | Oligo1 (antisense) | LINKER B | Oligo3 (sense) | 5′ |
| 3′ | Oligo3 (antisense) | LINKER C | Oligo2 (sense) | 5′ |
| or | | | | |
| 5′ | Oligo1 (sense) | LINKER A | Oligo3 (antisense) | 3′ |
| 3′ | Oligo1 (antisense) | LINKER B | Oligo2 (sense) | 5′ |
| 5′ | Oligo3 (sense) | LINKER C | Oligo2 (antisense) | 3′ |

wherein one or more of linker A, linker B or linker C is present; any combination of two or more oligonucleotides and one or more of linkers A-C is possible, so long as the polarity of the strands and the general structure of the molecule remains. Further, if two or more of linkers A-C are present, they may be identical or different.

Thus, a triple-armed structure is formed, wherein each arm comprises a sense strand and complementary antisense strand (i.e. Oligol antisense base pairs to Oligol sense etc.). The triple armed structure may be triple stranded, whereby each arm possesses base pairing.

Further, the above triple stranded structure may have a gap instead of a linker in one or more of the strands. Such a molecule with one gap is technically quadruple stranded and not triple stranded; inserting additional gaps or nicks will lead to the molecule having additional strands. Preliminary results obtained by the inventors of the present invention indicate that said gapped molecules are more active in inhibiting certain target genes than the similar but non-gapped molecules. This may also be the case for nicked molecules.

The compounds of the present invention can be synthesized by any of the methods that are well-known in the art for synthesis of ribonucleic (or deoxyribonucleic) oligonucleotides. Such synthesis is, among others, described in Beaucage S. L. and Iyer R. P., Tetrahedron 1992; 48: 2223-2311, Beaucage S. L. and Iyer R. P., Tetrahedron 1993; 49: 6123-6194 and Caruthers M. H. et. al., Methods Enzymol. 1987; 154: 287-313; the synthesis of thioates is, among others, described in Eckstein F., Annu. Rev. Biochem. 1985; 54: 367-402, the synthesis of RNA molecules is described in Sproat B., in Humana Press 2005 edited by Herdewijn P.; Kap. 2: 17-31 and respective downstream processes are, among others, described in Pingoud A. et al., in IRL Press 1989 edited by Oliver R. W. A.; Kap. 7: 183-208 and Sproat B., in Humana Press 2005 edited by Herdewijn P.; Kap. 2: 17-31 (supra).

Other synthetic procedures are known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Belton et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via a tandem synthesis methodology, as described in US patent application publication No. US2004/0019001 (McSwiggen), wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

### Pharmaceutical Compositions

While it may be possible for the compounds of the present invention to be administered as the raw chemical, it is preferable to present them as a pharmaceutical composition. Accordingly the present invention provides a pharmaceutical composition comprising one or more of the compounds of the invention; and a pharmaceutically acceptable carrier. This composition may comprise a mixture of two or more different oligonucleotides/siRNAs.

The invention further provides a pharmaceutical composition comprising at least one compound of the invention covalently or non-covalently bound to one or more compounds of the invention in an amount effective to inhibit one or more genes as disclosed above; and a pharmaceutically acceptable carrier. The compound may be processed intracellularly by endogenous cellular complexes to produce one or more oligoribonucleotides of the invention.

The invention further provides a pharmaceutical composition comprising a pharmaceutically acceptable carrier and one or more of the compounds of the invention in an amount effective to down-regulate expression in a cell of a target gene, the compound comprising a sequence substantially complementary to the sequence of (N)ₓ. In certain embodiments, the target gene is a viral, bacterial or mammalian gene. In various embodiments the target gene is a mammalian gene, preferably a human gene.

Additionally, the invention provides a method of inhibiting the expression of a target gene, by at least 50% as compared to a control, comprising contacting an mRNA transcript of the target gene with one or more of the compounds of the invention. In some embodiments an active siRNA compound inhibits gene expression at a level of at least 50%, 60% or 70% as compared to control. In certain preferred embodiments inhibition is at a level of at least 75%, 80% or 90% as compared to control. In some embodiments the target gene is a pro-apoptotic gene as disclosed herein.

In one embodiment the oligoribonucleotide is inhibiting one or more of the NOX genes of the present invention, whereby the inhibition is selected from the group comprising inhibition of gene function, inhibition of polypeptide and inhibition of mRNA expression.

In one embodiment the compound inhibits expression of a polypeptide encoded by a target gene whereby the inhibition is selected from the group comprising inhibition of function (which may be examined by an enzymatic assay or a binding assay with a known interactor of the native gene/polypeptide, inter alia), inhibition of protein (which may be examined by Western blotting, ELISA or immuno-precipitation, inter alia) and inhibition of mRNA expression (which may be examined by Northern blotting, quantitative RT-PCR, in-situ hybridisation or microarray hybridisation, inter alia).

In additional embodiments the invention provides a method of treating a subject suffering from a disease accompanied by an elevated level of the NOX genes of the present invention, the method comprising administering to the subject a compound of the invention in a therapeutically effective dose thereby treating the subject.

More particularly, the invention provides an oligoribonucleotide wherein one strand comprises consecutive nucleotides having, from 5′ to 3′, any one of the sequences set forth in Tables A-R or a homolog thereof wherein up to two of the ribonucleotides do not base pair with the ribonucleotide in the complementary strand

Additionally, further nucleic acids according to the present invention comprise at least 14 contiguous nucleotides of any one of the polynucleotides oligomers set forth in any one of Tables A-R and more preferably 14 contiguous nucleotide base pairs at any end of the double-stranded structure comprised of the first strand and second strand as described above.

### Methods of Treatment

In preferred embodiments the subject being treated is a warm-blooded animal and, in particular, mammals including human.

The term “treatment” as used herein refers to administration of a therapeutic substance to a subject in need thereof in an amount effective to ameliorate symptoms associated with a disease, to lessen the severity or cure the disease, or to prevent the disease from occurring.

Additionally, the invention provides a method of down-regulating the expression of a mammalian NOX genes selected from the group consisting of NOX4, NOX1, NOX2 (gp91phox, CYBB), NOX5, DUOX2, NOXO1, NOXO2, NOXA1 and NOXA2 (p67phox) by at least 50% as compared to a control comprising contacting a NOX mRNA transcript selected from the group consisting of NOX4, NOX1, NOX2 (gp91phox, CYBB), NOX5, DUOX2, NOXO1, NOXO2, NOXA1 and NOXA2 (p67phox), respectively with one or more of the compounds of the present invention.

In one embodiment the compound of the present invention is down-regulating one or more mammalian NOX genes selected from the group consisting of NOX4, NOX1, NOX2 (gp91phox, CYBB), NOX5, DUOX2, NOXO1, NOXO2, NOXA1 and NOXA2 (p67phox), whereby the down-regulation is selected from the group comprising down-regulation of gene function, down-regulation of polypeptide and down-regulation of mRNA expression.

In one embodiment the compound is down-regulating a mammalian NOX polypeptide, whereby the down-regulation is selected from the group comprising down-regulation of function (which may be examined by an enzymatic assay or a binding assay with a known interactor of the native gene/polypeptide, inter alia), down-regulation of protein (which may be examined by Western blotting, ELISA or immuno-precipitation, inter alia) and down-regulation of mRNA expression (which may be examined by Northern blotting, quantitative RT-PCR, in-situ hybridisation or microarray hybridisation, inter alia).

In additional embodiments the invention provides a method of treating a patient suffering from a disease accompanied by an elevated level of a mammalian NOX gene, the method comprising administering to the patient a compound or composition of the invention in a therapeutically effective dose thereby treating the patient.

The present invention relates to the use of compounds which down-regulate the expression of a mammalian NOX gene particularly to novel small interfering RNAs (siRNAs), in the treatment of the following diseases or conditions in which inhibition of the expression of the mammalian NOX gene is beneficial: hearing loss, acute renal failure, ocular disease e.g. glaucoma, Acute Respiratory Distress Syndrome and other acute lung injuries, lung transplantation, spinal cord injury, pressure sores, osteoarthritis and Chronic Obstructive Pulmonary Disease (COPD). Other indications include chemical-induced nephrotoxicity and chemical-induced neurotoxicty, for example toxicity induced by cisplatin and cispaltin-like compounds, by aminoglycosides, by loop diuretics, and by hydroquinone and their analogs.

Methods, molecules and compositions which inhibit the mammalian NOX gene or polypeptide are discussed herein at length, and any of said molecules and/or compositions may be beneficially employed in the treatment of a patient suffering from any of said conditions. In certain preferred embodiments the NOX gene is NOX4. A list of siRNAs directed to NOX4 gene are listed in Table A, infra. The number in parenthesis (#) indicates the number of the compound in the respective table, supra.

Certain preferred NOX4 siRNA compounds include:

| | |
|---|---|
| NOX4 (#4 TABLE A): | |
| Antisense 5′-3′ | UCUAGAUUGAAUGAAGGGC |
| Sense 3′-5′ | AGAUCUAACUUACUUCCCG |
| NOX4 (#55 TABLE A): | |
| Antisense 5′-3′ | UUUCUCCAAUUAUCUUCUG |
| Sense 3′-5′ | AAAGAGGUUAAUAGAAGAC |
| NOX4 (#184 TABLE A): | |
| Antisense 5′-3′ | AAGUCUUAGAAAUUGCACU |
| Sense: 3′-5′ | UUCAGAAUCUUUAACGUGA |

Certain preferred NOX2 (CYBB) siRNA compounds include:

| | |
|---|---|
| CYBB (#6 TABLE E) | |
| Antisense 5′-3′ | AAUUCAUUAUCCCAGUUGG |
| Sense 3′-5′ | UUAAGUAAUAGGGUCAACC |
| CYBB (#7 TABLE E) | |
| Antisense 5′-3′ | AAUGACCAAUAUGAGACCC |
| Sense 3′-5′ | UUACUGGUUAUACUCUGGG |
| CYBB (#2 TABLE E) | |
| Antisense 5′-3′ | UUAUGUCCUAAUGAUCUGG |
| Sense 3′-5′ | AAUACAGGAUUACUAGACC |
| CYBB (#1, TABLE E) | |
| Antisense 5′-3′ | AAGAACAAAAAGAAAACCC |
| Sense 3′-5′ | UUCUUGUUUUUGUUUUGGG |

Certain preferred NOX1 siRNA compounds include:

| | |
|---|---|
| NOX1 (#3 TABLE C) | |
| Antisense 5′-3′ | AACAGGAAAACAUUCAGCC |
| Sense 3′-5′ | UUGUCCUUUUGUAAGUCGG |
| NOX1 (#1 TABLE C) | |
| Antisense 5′-3′ | UUAAAAUUCAAGCAGAGAG |
| Sense 3′-5′ | AAUUUUAAGUUCGUCUCUC |
| NOX1 (#60 TABLE C) | |
| Antisense 5′-3′ | GUGUAGUAGUAUUUGUCGG |
| Sense 3′-5′ | CACAUCAUCAUAAACAGCC |
| NOX1 (#17 TABLE C) | |
| Antisense 5′-3′ | AUCAUGAGAUAGGCUGGAG |
| Sense 3′-5′ | UAGUACUCUAUCCGACCUC |
| NOX1 (#26 TABLE C) | |
| Antisense 5′-3′ | UCAAAGUUAAACAGGUGUG |
| Sense 3′-5′ | AGUUUCAAUUUGUCUACAC |

Certain preferred NOXO1 siRNA compounds include:

| | |
|---|---|
| NOXO1 (#4 TABLE K) | |
| Antisense 5′-3′ | UUUGGAGCCUCUUGAUCUG |
| Sense 3′-5′ | AAACCUCGGAGAACUAGAC |
| NOXO1 (#2 TABLE K) | |
| Antisense 5′-3′ | UCUGACGUUUCCAACACGC |
| Sense 3′-5′ | AGACUGCAAAGGUUGUCAG |
| NOXO1 (#7 TABLE K) | |
| Antisense 5′-3′ | AGAAUAGGUUUCCAACAGC |
| Sense 3′-5′ | UCUUAUCCAAAGGUUGUCG |
| NOXO1 (#10 TABLE K) | |
| Antisense 5′-3′ | AGAAGGCAAACGUUUGGAG |
| Sense 3′-5′ | UCUUCCGUUUGCAAACCUC |
| NOXO1 (#8 TABLE K) | |
| Antisense 5′-3′ | CUGAAUUCGUCCCAACUCC |
| Sense 3′-5′ | GACUUAAGCAGGGUUGAGG |

“Treatment” refers to both therapeutic treatment and prophylactic or preventative measures, wherein the object is to prevent or slow down (lessen) NOX-related disorder as listed above. Those in need of treatment include those already experiencing the disease or condition, those prone to having the disease or condition, and those in which the disease or condition is to be prevented. The compounds of the invention may be administered before, during or subsequent to the onset of the disease or condition.

The method of the invention includes administering a therapeutically effective amount of one or more compounds which down-regulate expression of the NOX gene, particularly the novel siRNAs of the present invention, small molecule inhibitors of the NOX gene or protein or antibodies to the NOX proteins.

In some preferred embodiments, the methods of the invention are applied to various conditions of bearing loss. Without being bound by theory, the hearing loss may be due to inner ear hair cell damage or loss, wherein the damage or loss is caused by, inter alia, infection, mechanical injury, loud sound, aging (presbycusis or loss of hearing that gradually occurs in most individuals as they grow older), or chemical-induced ototoxicity. Ototoxins include therapeutic drugs including antineoplastic agents, salicylates, quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. Preferably a therapeutically effective composition is given immediately after the exposure to prevent or reduce the ototoxic effect. More preferably, treatment is provided prophylactically, either by administration of the composition prior to or concomitantly with the ototoxic pharmaceutical or the exposure to the ototoxin.

By “ototoxin” in the context of the present invention is meant a substance that through its chemical action injures, impairs or inhibits the activity of the sound receptors component of the nervous system related to hearing, which in turn impairs hearing (and/or balance). In the context of the present invention, ototoxicity includes a deleterious effect on the inner ear hair cells Ototoxic agents that cause hearing impairments include, but are not limited to, neoplastic agents such as vincristine, vinblastine, cisplatin and cisplatin-like compounds, taxol and taxol-like compounds, dideoxy-compounds, e.g., dideoxyinosine; alcohol; metals; industrial toxins involved in occupational or environmental exposure; contaminants of food or medicinals; and over-doses of vitamins or therapeutic drugs, e.g., antibiotics such as penicillin or chloramphenicol, and megadoses of vitamins A, D, or B6, salicylates, quinines and loop diuretics.

The ototoxic effects of various therapeutic drugs on auditory cells and spiral ganglion neurons are often the limiting factor for their therapeutic usefulness. Main ototoxic drugs include the widely used chemotherapeutic agent cisplatin and its analogs, commonly used aminoglycoside antibiotics, e.g. gentamicin, for the treatment of infections caused by gram-negative bacteria, quinine and its analogs, salicylate and its analogs, and loop-diuretics.

For example, antibacterial aminoglycosides such as gentamicins, streptomycins, kanamycin, tobramycins, and the like are known to have serious toxicity, particularly ototoxicity and nephrotoxicity, which reduce the value of such antimicrobials as therapeutic agents (see Goodman and Gilman's The Pharmacological Basis of Therapeutics, 6th ed., A. Goodman Gilman et al., eds; Macmillan Publishing Co., Inc., New York, 1980, pp. 1169-71).

Clearly, ototoxicity is a dose-limiting side-effect of antibiotic administration. Studies have shown that from 4% to 15% of patients receiving 1 gram per day for greater than 1 week develop measurable hearing loss, which slowly becomes worse and can lead to complete permanent deafness if treatment continues.

Nephrotoxicity and ototoxicity are serious dose-limiting side-effect for cisplatin, a platinum coordination complex, that has proven effective on a variety of human cancers including testicular, ovarian, bladder, and head and neck cancer. Cisplatin (Platinol®) damages auditory and vestibular systems. Salicylates, such as aspirin, are the most commonly used therapeutic drugs for their anti-inflammatory, analgesic, anti-pyretic and anti-thrombotic effects. Unfortunately, they too have ototoxic side effects and can lead to tinnitus (“ringing in the ears”) and temporary hearing loss. Moreover, if the drug is used at high doses for a prolonged time, chronic and irreversible hearing impairment can become an issue. Another target organ for cisplatin toxicity is the kidney. This toxicity is manifested by reduced renal function and leads to serum electrolyte changes and pathological changes in the urine analysis. Doses of cisplatin, which produce changes in renal function may cause no histopathological changes. Higher doses of the drug lead to terstitial nephritis. Cisplatin also causes bone marrow hypoplasia, and can cause autonomic neuropathy. Slight changes in liver function and histopathology are also observed following cisplatin therapy.

Without being bound by theory, it is believed that cisplatin drugs and other potentially ototoxic drugs (such as aminoglycoside antibiotics) may induce the ototoxic effects via programmed cell death or apoptosis in inner ear tissue, particularly inner ear hair cells (Zhang et al., Neuroscience 2003, 120(1):191-205; Wang et al., J. Neuroscience 2003, 23(24):8596-8607). In mammals, auditory hair cells are produced only during embryonic development and do not regenerate if lost during postnatal life, therefore, a loss of hair cells will result in profound and irreversible deafness. Unfortunately, at present, there are no effective therapies to treat the cochlea and reverse this condition. Thus, an effective therapy to prevent cell death of auditory hair cells would be of great therapeutic value.

Another type of hearing loss is presbycusis, which is age related hearing loss. It is estimated that about 30-35 percent of adults between the ages of 65 and 75 years and about 40-50 percent of people aged 75 and older have hearing loss. Accordingly, there exists a need for means to prevent, reduce or treat the incidence and/or severity of inner ear disorders and hearing impairments involving inner ear tissue, particularly inner ear hair cells.

By “exposure to an toxic agent” is meant that the toxic agent is made available to, or comes into contact with, a mammal. A toxic agent can be toxic to one or more organs in the body, for example, the ear, kidney, nervous system, liver and the like. Exposure to an toxic agent can occur by direct administration, e.g., by ingestion or administration of a food, medicinal, or therapeutic agent, e.g., a chemotherapeutic agent, by accidental contamination, or by environmental exposure, e.g., aerial or aqueous exposure.

Hearing loss relevant to the invention may be due to end-organ lesions involving inner ear hair cells, e.g., acoustic trauma, viral endolymphatic labyrinthitis, Meniere's disease. Hearing impairments include tinnitus, which is a perception of sound in the absence of an acoustic stimulus, and may be intermittent or continuous, wherein there is diagnosed a sensorineural loss. Hearing loss may be due to bacterial or viral infection, such as in herpes zoster oticus, purulent labyrinthitis arising from acute otitis media, purulent meningitis, chronic otitis media, sudden deafness including that of viral origin, e.g., viral endolymphatic labyrinthitis caused by viruses including mumps, measles, influenza, chicken pox, mononucleosis and adenoviruses. The hearing loss can be congenital, such as that caused by rubella, anoxia during birth, bleeding into the inner ear due to trauma during delivery, ototoxic drugs administered to the mother, erythroblastosis fetalis, and hereditary conditions including Waardenburg's syndrome and Hurler's syndrome.

The hearing loss can be noise-induced, generally due to a noise, greater than about 85 decibels (db) that damages the inner ear. In a particular aspect of the invention, the hearing loss is caused by an ototoxic drug that effects the auditory portion of the inner ear, particularly inner ear hair cells. Incorporated herein by reference are chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210 relating to description and diagnosis of hearing and balance impairments.

It is the object of the present invention to provide a method and compositions for treating a mammal, to prevent, reduce, or treat a hearing impairment, disorder or imbalance, preferably an ototoxin-induced hearing condition, by administering to a mammal in need of such treatment a composition of the invention. One embodiment of the invention is a method for treating a hearing disorder or impairment wherein the ototoxicity results from administration of a therapeutically effective amount of an ototoxic pharmaceutical drug. Typical ototoxic drugs are chemotherapeutic agents, e.g. antineoplastic agents, and antibiotics. Other possible candidates include loop-diuretics, quinines or a quinine-like compound, and salicylate or salicylate-like compounds.

Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2.

Ototoxic chemotherapeutic agents amenable to the methods of the invention include, but are not limited to an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas. Cisplatin-like compounds include carboplatin (Paraplatin tetraplatin, oxaliplatin, aroplatin and transplatin inter alia.

The methods and compositions of the present invention are also effective in the treatment of acoustic trauma or mechanical trauma, preferably acoustic or mechanical trauma that leads to inner ear hair cell loss. Acoustic trauma to be treated in the present invention may be caused by a single exposure to an extremely loud sound, or following long-term exposure to everyday loud sounds above 85 decibels. Mechanical inner ear trauma to be treated in the present invention is for example the inner ear trauma following insertion and operation of an electronic device in the inner ear. The compositions of the present invention prevent or minimize the damage to inner ear hair cells associated with the device.

In some embodiments the composition of the invention is co-administered with an ototoxin. For example, the present invention provides an improved method for treatment of infection of a mammal receiving an antibiotic for treatment of the infection, comprising administering a therapeutically effective amount of one or more compounds (particularly novel siRNAs) which down-regulate expression of the mammalian NOX4 gene, to the patient in need of such treatment to reduce or prevent ototoxin-induced hearing impairment associated with the antibiotic. The compounds which down-regulate expression of a NOX gene, in particular novel NOX4 siRNA compounds are preferably administered locally within the inner ear.

In yet another embodiment an improved method for treatment of cancer in a mammal by administration of a chemotherapeutic compound is provided, wherein the improvement comprises administering a therapeutically effective amount of a composition of the invention to the patient in need of such treatment to reduce or prevent ototoxin-induced hearing impairment associated with the chemotherapeutic drug. The compounds which reduce or prevent the ototoxin-induced hearing impairment, eg. the novel siRNAs inter alia are preferably administered locally within the inner ear.

In another embodiment the methods of treatment are applied to treatment of hearing loss resulting from the administration of a chemotherapeutic agent in order to treat its ototoxic side-effect.

In another embodiment the methods of the invention are applied to hearing impairments resulting from the administration of quinine and its synthetic substitutes, typically used in the treatment of malaria, to treat its ototoxic side-effect.

In another embodiment the methods of the invention are applied to hearing impairments resulting from administration of a diuretic to treat its ototoxic side-effect. Diuretics, particularly “loop” diuretics, i.e. those that act primarily in the Loop of Henle, are candidate ototoxins. Illustrative examples, not limiting to the invention method, include furosemide, ethacrylic acid, and mercurials. Diuretics are typically used to prevent or eliminate edema. Diuretics are also used in nonedematous states for example hypertension, hypercalcemia, idiopathic hypercalciuria, and nephrogenic diabetes insipidus.

In another preferred embodiment, the compounds of the invention are used for treating acute renal failure, in particular acute renal failure due to ischemia in post surgical patients, and acute renal failure due to chemotherapy treatment such as cisplatin administration or sepsis-associated acute renal failure. A preferred use of the compounds of the invention is for the prevention of acute renal failure in high-risk patients undergoing major cardiac surgery or vascular surgery. The patients at high-risk of developing acute renal failure can be identified using various scoring methods such as the Cleveland Clinic algorithm or that developed by US Academic Hospitals (QMMI) and by Veterans' Administration (CICSS). Other preferred uses of the compounds of the invention are for the prevention of ischemic acute renal failure in kidney transplant patients or for the prevention of toxic ARF in patients receiving chemotherapy.

In another preferred embodiment, the compounds of the invention are used for treating ocular diseases (e.g. glaucoma, ocular ischemic conditions and dry eye syndrome).

In other embodiments the compounds and methods of the invention are useful for treating or preventing the incidence or severity of various diseases and conditions in a patient, in particular conditions which are result from ischemic/reperfusion injury or oxidative stress, Acute Respiratory Distress Syndrome (ARDS) for example due to coronavirus infection or endotoxins, severe acute respiratory syndrome (SARS), and other acute lung injuries, ischemia reperfusion injury associated with hmg transplantation, glaucoma, spinal cord injury, pressure sores, osteoarthritis and Chronic Obstructive Pulmonary Disease (COPD). The methods comprising administering to the patient a composition comprising one or more inhibitors (such as siRNA compounds) which inhibit NOX4 gene in a therapeutically effective dose, thereby treating the patient.

In other embodiments the compounds and methods of the invention are useful for treating or preventing the incidence or severity of other diseases and conditions in a patient. These diseases and conditions include stroke and stroke-like situations (e.g. cerebral, renal, cardiac failure), neuronal cell death, brain injuries with or without reperfusion, chronic degenerative diseases e.g. neurodegenerative disease including Alzheimer's disease, Huntington's disease, Parkinson's disease, multiple sclerosis, amyotrophic lateral sclerosis, spinobulbar atrophy, prion disease, and apoptosis resulting from traumatic brain injury (TBI).

The compounds and methods of the invention are directed to providing neuroprotection, cerebroprotection, or to prevent and/or treat cytotoxic T cell and natural killer cell-mediated apoptosis associated with autoimmune disease and transplant rejection, or to prevent cell death of cardiac cells including heart failure, cardiomyopathy, viral infection or bacterial infection of the heart, myocardial ischemia, myocardial infarct, and myocardial ischemia, coronary artery by-pass graft, or to prevent and/or treat mitochondrial drug toxicity e.g. as a result of chemotherapy or HIV therapy, to prevent cell death during viral infection or bacterial infection, or to prevent and/or treat inflammation or inflammatory diseases, inflammatory bowel disease, sepsis and septic shock. Other uses include prevention of cell death from follicle to ovocyte stages, from ovocyte to mature egg stages and sperm (for example, methods of freezing and transplanting ovarian tissue, artificial fertilization), or to preserve fertility in mammals after chemotherapy, in particular human mammals, or to prevent and/or treat, macular degeneration, or to prevent and/or treat acute hepatitis, chronic active hepatitis, hepatitis-B, and hepatitis-C, or to prevent hair loss, (e.g. hair loss due-to male-pattern baldness, or hair loss due to radiation, chemotherapy or emotional stress), or to treat or ameliorate skin damage whereby the skin damage may be due to exposure to high levels of radiation, heat, chemicals, sun, or to burns and autoimmune diseases), or to prevent cell death of bone marrow cells in myelodysplastic syndromes (MDS), or to treat pancreatisis, or to treat, rheumatoid arthritis, psoriasis, glomerulonephritis, atheroscerosis, and graft versus host disease (GVHD), or to treat retinal pericyte apoptosis, retinal damages resulting from ischemia, diabetic retinopathy, or to treat any disease states associated NOX expression, wherein the NOX expression is associated with expression of NOX1, NOX2 (gp91phox, CYBB), NOX4, NOX5, DUOX2, NOXO1, NOXO2, NOXA1 and/or NOXA2 (p67phox).

The present invention also relates to organ transplantation in general. For organ transplantation, either the donor or the recipient or both may be treated with a compound or composition of the present invention. Accordingly, the present invention relates to a method of treating an organ donor or an organ recipient comprising the step of administering to the organ donor or organ recipient a therapeutically effective amount of a compound according to the present invention.

The invention further relates to a method for preserving an organ comprising contacting the organ with an effective amount of compound of the present invention. Also provided is a method for reducing or preventing injury (in particular reperfusion injury) of an organ during surgery and/or following removal of the organ from a subject comprising placing the organ in an organ preserving solution wherein the solution comprises a compound according to the present invention.

The present invention also provides for a process of preparing a pharmaceutical composition, which comprises:
providing one or more double stranded compound of the invention; and
admixing said compound with a pharmaceutically acceptable carrier.

In a preferred embodiment, the compound used in the preparation of a pharmaceutical composition is admixed with a carrier in a pharmaceutically effective dose. In a particular embodiment the compound of the present invention is conjugated to a steroid or to a lipid or to another suitable molecule e.g. to cholesterol.

### Delivery

The siRNA molecules of the present invention may be delivered to the target tissue by direct application of the naked molecules prepared with a carrier or a diluent.

The term “naked siRNA” refers to siRNA molecules that are free from any delivery vehicle that acts to assist, promote or facilitate entry into the cell, including viral sequences, viral particles, liposome formulations, lipofectin or precipitating agents and the like. For example, siRNA in PBS is “naked siRNA”.

However, in some embodiments the siRNA molecules of the invention are delivered in liposome formulations and lipofectin formulations and the like and can be prepared by methods well known to those skilled in the art. Such methods are described, for example, in U.S. Pat. Nos. 5,593,972, 5,589,466, and 5,580,859, which are herein incorporated by reference.

Delivery systems aimed specifically at the enhanced and improved delivery of siRNA into mammalian cells have been developed (see, for example, Shen et al FEBS Let. 539: 111-114 (2003), Xia et al., Nat. Biotech. 20: 1006-1010 (2002), Reich et al., Mol. Vision 9: 210-216 (2003), Sorensen et al., J. Mol. Biol. 327: 761-766 (2003), Lewis et al., Nat. Gen. 32: 107-108 (2002) and Simeoni et al., NAR 31, 11: 2717-2724 (2003)). siRNA has recently been successfully used for inhibition of gene expression in primates; for details see for example, Tolentino et al., Retina 24(1) February 2004 1132-138. Respiratory formulations for siRNA are described in US patent application publication No. 2004/0063654. Cholesterol-conjugated siRNAs (and other steroid and lipid conjugated siRNAs) can been used for delivery (see for example Soutschek et al Nature 432: 173-177(2004) Therapeutic silencing of an endogenous gene by systemic administration of modified siRNAs; and Lorenz et al. Bioorg. Med. Chem. Lett. 14:4975-4977 (2004) Steroid and lipid conjugates of siRNAs to enhance cellular uptake and gene silencing in liver cells.)

Pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention and they include liposomes and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art. In one specific embodiment of this invention topical and transdermal formulations may be selected. The siRNAs or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

A “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art.

In general, the active dose of compound for humans is in the range of from 1 ng/kg to about 100 mg/kg body weight per day, preferably about 0.01 mg to about 2-10 mg/kg body weight per day, in a regimen of one dose per day or twice or three or more times per day for a period of 1-4 weeks or longer.

The compounds of the present invention can be administered by any of the conventional mutes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental mutes of administration. The liquid compositions include aqueous solutions, with and without organic co-solvents, aqueous or oil suspensions, emulsions with edible oils, as well as similar pharmaceutical vehicles. In a particular embodiment, the administration comprises intravenous administration. In another embodiment the administration comprises topical or local administration

In addition, in certain embodiments the compositions for use in the novel treatments of the present invention may be formed as aerosols, for example for intranasal administration, or oral compositions (such as tablets, suspensions, solutions) for local delivery to the oral cavity.

The present invention further provides for a pharmaceutical composition comprising two or more siRNA molecules for the treatment of any of the diseases and conditions mentioned herein, whereby said two molecules may be physically mixed together in the pharmaceutical composition in amounts which generate equal or otherwise beneficial activity, or may be covalently or non-covalently bound, or joined together by a nucleic acid linker of a length ranging from 2-100, preferably 2-50 or 2-30 nucleotides. In one embodiment, the siRNA molecules are comprised of a double-stranded nucleic acid structure as described herein, wherein the two siRNA sequences are selected from Tables A-R.

The siRNA molecules are covalently or non-covalently bound or joined by a linker to form a tandem siRNA molecule. Such tandem siRNA molecules comprising two siRNA sequences are typically of 38-150 nucleotides in length, more preferably 38 or 40-60 nucleotides in length, and longer accordingly if more than two siRNA sequences are included in the tandem molecule. A longer tandem molecule comprised of two or more longer sequences which encode siRNA produced via internal cellular processing, e.g., long dsRNAs, is also envisaged, as is a tandem molecule encoding two or more shRNAs. Such tandem molecules are also considered to be a part of the present invention.

siRNA molecules that target the NOX gene may be the main active component in a pharmaceutical composition, or may be one active component of a pharmaceutical composition containing two or more siRNAs (or molecules which encode or endogenously produce two or more siRNAs, be it a mixture of molecules or one or more tandem molecules which encode two or more siRNAs), said pharmaceutical composition further being comprised of one or more additional siRNA molecule which targets one or more additional gene. Simultaneous inhibition of said additional gene(s) will likely have an additive or synergistic effect for treatment of the diseases disclosed herein.

Additionally, the NOX siRNA compounds disclosed herein or any nucleic acid molecule comprising or encoding such siRNA can be linked or bound (covalently or non-covalently) to antibodies (including aptamer molecules) against cell surface internalizable molecules expressed on the target cells, in order to achieve enhanced targeting for treatment of the diseases disclosed herein. For example, anti-Fas antibody (preferably a neutralizing antibody) may be combined (covalently or non-covalently) with NOX siRNA. In another example, an aptamer which can act like a ligand/antibody may be combined (covalently or non-covalently) with any NOX siRNA.

The compounds of the present invention can be delivered either directly or with viral or non-viral vectors. When delivered directly the sequences are generally rendered nuclease resistant. Alternatively the sequences can be incorporated into expression cassettes or constructs such that the sequence is expressed in the cell as discussed herein below. Generally the construct contains the proper regulatory sequence or promoter to allow the sequence to be expressed in the targeted cell. Vectors optionally used for delivery of the compounds of the present invention are commercially available, and may be modified for the purpose of delivery of the compounds of the present invention by methods known to one of skill in the art.

It is also envisaged that a long oligonucleotide (typically 25-500 nucleotides in length) comprising one or more stern and loop structures, where stem regions comprise the sequences of the oligonucleotides of the invention, may be delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the invention. This oligonucleotide can be termed a tandem shRNA construct. It is envisaged that this long oligonucleotide is a single stranded oligonucleotide comprising one or more stem and loop structures, wherein each stem region comprises a sense and corresponding antisense siRNA sequence of the NOX genes of the invention. In particular, it is envisaged that this oligonucleotide comprises sense and antisense siRNA sequences as depicted in Tables A-R.

Although the inhibitor may be an siRNA molecule, other inhibitors contemplated to be used in the methods of the invention to inhibit a NOX gene and to treat the diseases and conditions described herein are inter alia antibodies, preferably neutralizing antibodies or fragments thereof, including single chain antibodies, antisense oligonucleotides, antisense DNA or RNA molecules, ribozymes, proteins, polypeptides and peptides including peptido-mimetics and dominant negatives, and also expression vectors expressing all the above. Additional inhibitors may be small chemical molecules, which generally have a molecular weight of less than 2000 daltons, more preferably less than 1000 daltons, even more preferably less than 500 daltons. These inhibitors may act as follows: small molecules may affect expression and/or activity; antibodies may affect activity; all kinds of antisense may affect NOX4 gene expression; and dominant negative polypeptides and peptidomimetics may affect activity; expression vectors may be used inter alia for delivery of antisense or dominant-negative polypeptides or antibodies.

### Antibodies

The term “antibody” refers to IgG, IgM, IgD, IgA, and IgE antibody, inter alia. The definition includes polyclonal antibodies or monoclonal antibodies. This term refers to whole antibodies or fragments of antibodies comprising an antigen-binding domain, e.g. antibodies without the Fc portion, single chain antibodies, miniantibodies, fragments consisting of essentially only the variable, antigen-binding domain of the antibody, etc. The term “antibody” may also refer to antibodies against polynucleotide sequences obtained by cDNA vaccination. The term also encompasses antibody fragments which retain the ability to selectively bind with their antigen or receptor and are exemplified as follows, inter alia:
(1) Fab, the fragment which contains a monovalent antigen-binding fragment of an antibody molecule which can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′₂) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

### Antisense Molecules

By the term “antisense” (AS) or “antisense fragment” is meant a polynucleotide fragment (comprising either deoxyribonucleotides, ribonucleotides or a mixture of both) having inhibitory antisense activity, said activity causing a decrease in the expression of the endogenous genomic copy of the corresponding gene. Many reviews have covered the main aspects of antisense (AS) technology and its therapeutic potential (Aboul-Fadi T., Curr Med Chem. 2005, 12(19):2193-214; Crooke S T, Curr Mol Med. 2004, 4(5):465-87; Crooke S T, Ann Rev Med. 2004, 55:61-95; Vacek M et al., Cell Mol Life Sci. 2003, 60(5):825-33; Cho-Chung Y S, Arch Pharm Res. 2003, 26(3):183-91. Additional reviews on the chemical (Crooke et al., Hematol Pathol. 1995, 9(2):59-72), cellular (Wagner, Nature. 1994, 372(6504):333-5) and therapeutic (Scanlon, et al, FASEB J. 1995, 9(13):1288-96) aspects of AS technology are available. Antisense intervention in the expression of specific genes can be achieved by the use of modified AS oligonucleotide sequences (for recent reports see Lefebvre-d'Hellencourt et al, 1995; Agrawal, 1996; LevLehman et al, 1997).

AS oligonucleotide sequences may be short sequences of DNA, typically 15-30 mer but may be as small as 7-mer (Wagner et al, Nat Biotech. 1996, 14(7):840-4), designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, Semin Oncol. 1996, 23(1):78-87). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive.

The sequence target segment for the antisense oligonucleotide is selected such that the sequence exhibits suitable energy related characteristics important for oligonucleotide duplex formation with their complementary templates, and shows a low potential for self-dimerization or self-complementation (Anazodo et al., 1996, Biochem. Biophys. Res. Comm. 229:305-309). For example, the computer program OLIGO (Primer Analysis Software, Version 3.4), can be used to determine antisense sequence melting temperature, free energy properties, and to estimate potential self-dimer formation and self-complimentary properties. The program allows the determination of a qualitative estimation of these two parameters (potential self-dimer formation and self-complimentary) and provides an indication of “no potential” or “some potential” or “essentially complete potential”. Using this program target segments are generally selected that have estimates of no potential in these parameters. However, segments can be used that have “some potential” in one of the categories. A balance of the parameters is used in the selection as is known in the art. Further, the oligonucleotides are also selected as needed so that analog substitution does not substantially affect function.

Phosphorothioate antisense oligonucleotides do not normally show significant toxicity at concentrations that are effective and exhibit sufficient pharmacodynamic half-lives in animals (Agrawal, et al., PNAS USA. 1997, 94(6):2620-5) and are nuclease resistant. Antisense oligonucleotide inhibition of basic fibroblast growth factor (bFGF), having mitogenic and angiogenic properties, suppressed 80% of growth in glioma cells (Morrison, J Biol Chem. 1991 266(2):728-34) in a saturable and specific manner. Being hydrophobic, antisense oligonucleotides interact well with phospholipid membranes (Akhter et al., NAR. 1991, 19:5551-5559). Following their interaction with the cellular plasma membrane, they are actively (or passively) transported into living cells (Loke et al., PNAS 1989, 86(10):3474-8), in a saturable mechanism predicted to involve specific receptors (Yakubov at al., PNAS, 1989 86(17):6454-58).

### Ribozymes

A “ribozyme” is an RNA molecule that possesses RNA catalytic ability (see Cech for review) and cleaves a specific site in a target RNA. In accordance with the present invention, ribozymes which cleave mRNA may be utilized as inhibitors. This may be necessary in cases where antisense therapy is limited by stoichiometric considerations (Sarver et al., 1990, Gene Regulation and Aids, pp. 305-325). Ribozymes can then be used that will target the a gene associated with a bone marrow disease. The number of RNA molecules that are cleaved by a ribozyme is greater than the number predicted by stochiochemistry. (Hampel and Tritz, Biochem. 1989, 28(12):4929-33; Uhlenbeck, Nature. 1987 328(6131):596-600).

Ribozymes catalyze the phosphodiester bond cleavage of RNA. Several ribozyme structural families have been identified including Group I introns, RNase P, the hepatitis delta virus ribozyme, hammerhead ribozymes and the hairpin ribozyme originally derived from the negative strand of the tobacco ringspot virus satellite RNA (sTRSV) (U.S. Pat. No. 5,225,347). The latter two families are derived from viroids and virusoids, in which the ribozyme is believed to separate monomers from oligomers created during rolling circle replication (Symons, 1989 and 1992). Hammerhead and hairpin ribozyme motifs are most commonly adapted for trans-cleavage of mRNAs for gene therapy (Sullivan, 1994). In general the ribozyme has a length of from about 30-100 nucleotides. Delivery of ribozymes is similar to that of AS fragments and/or siRNA molecules.

### Screening for NOX Inhibitors

Some of the compounds and compositions of the present invention may be used in a screening assay for identifying and isolating compounds that modulate the activity of a NOX gene, in particular compounds that modulate a disorder accompanied by an elevated level of a NOX gene. The compounds to be screened comprise inter alia substances such as small chemical molecules and antisense oligonucleotides.

The inhibitory activity of the compounds of the present invention on a NOX gene or binding of the compounds of the present invention to a NOX gene may be used to determine the interaction of a test compound with the NOX gene, e.g., if the test compound competes with the oligonucleotides of the present invention for inhibition of a NOX gene, or if the additional compound rescues said inhibition. The inhibition or activation can be tested by various means, such as, inter alia, assaying for the product of the activity of the NOX polypeptide or displacement of binding compound from the NOX polypeptide in radioactive or fluorescent competition assays.

The present invention is illustrated in detail below with reference to Examples, but is not to be construed as being limited thereto.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### EXAMPLES

### General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and as in Ausubel et al., Current Protocols in Molecular Biology, John Wiley and Sons, Baltimore, Md. (1989) and as in Perbal, A Practical Guide to Molecular Cloning, John Wiley & Sons, New York (1988), and as in Watson et al, Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In situ PCR in combination with Flow Cytometry can be used for detection of cells containing specific DNA and mRNA sequences (Testoni et al., 1996, Blood 87:3822.) Methods of performing RT-PCR are also well known in the art.

### Example 1

### In Vitro Testing of the siRNA Compounds for NOX Genes

### 1. General

About 1.5-2×10⁵ tested cells (HeLa cells or 293T cells for siRNA targeted the human gene and NRKS2 cells or NMUMG cells for siRNA targeted the rat/mouse gene) were seeded per well in 6 wells plate (70-80% confluent).

At 24 h subsequently, cells were transfected with siRNA compounds using Lipofectamine® 2000 reagent (Invitrogene) at final concentration of 500 pM, 5 nM, 20 nM or 40 nM. The cells were incubated at 37° C. in a CO2 incubator for 72 h.

As positive control for cells transfection PTEN-Cy3 labeled sRNA compounds were used. As negative control for siRNA activity GFP siRNA compounds were used.

At 72 h after transfection cells were harvested and RNA was extracted from cells. Transfection efficiency was tested by fluorescent microscopy.

Results: The percent of inhibition of gene expression using specific preferred siRNAs was determined using qPCR analysis of target gene in cells expressing the endogenous gene. The data demonstrate the percent of knockdown of the expression of the target gene in cells. In general, the siRNAs having specific sequences that were selected for in vitro testing were specific for both human and the rat/rabbit genes. Similar results of reduced expression of specific genes are obtained with other siRNAs, the sequences of which are listed in Tables A-R.

### Example 2

### Model Systems of Acute Renal Failure (ARF)

ARF is a clinical syndrome characterized by rapid deterioration of renal function that occurs within days. Without being bound by theory acute kidney injury may be the result of renal ischemia-reperfusion injury such as renal ischemia-reperfusion injury in patients undergoing major surgery such as major cardiac surgery. The principal feature of ARF is an abrupt decline in glomerular filtration rate (GFR), resulting in the retention of nitrogenous wastes (urea, creatinine). Recent studies, support that apoptosis in renal tissues is prominent in most human cases of ARF. The principal site of apoptotic cell death is the distal nephron. During the initial phase of ischemic injury, loss of integrity of the actin cytoskeleton leads to flattening of the epithelium, with loss of the brush border, loss of focal cell contacts, and subsequent disengagement of the cell from the underlying substratum.

Testing the active siRNA for each NOX gene separately for treating ARF is done using an animal model for ischemia-reperfusion-induced ARF.

Ischemia-Reperfusion Induced ARF

Ischemia-reperfusion injury is induced in rats following 45 minutes bilateral kidney arterial clamp and subsequent release of the clamp to allow 24 hours of reperfusion. Twelve mg/kg of siRNA to a specific NOX gene are injected into the jugular vein 30 minutes prior to and 4 hours following the clamp. ARF progression is monitored by measurement of serum creatinine levels before (baseline) and 24 hrs post surgery. At the end of the experiment, the rats are perfused via an indwelling femoral line with warm PBS followed by 4% paraformaldehyde. The left kidneys are removed and stored in 4% paraformaldehyde for subsequent histological analysis. Acute renal failure is frequently defined as an acute increase of the serum creatinine level from baseline. An increase of at least 0.5 mg per dL or 44.2 μmol per L of serum creatinine is considered as an indication for acute renal failure. Serum creatinine is measured at time zero before the surgery and at 24 hours post ARF surgery. The results show that siRNA to each specific NOX gene described herein prevents production of acute renal failure in this model.

### Example 3

### Model Systems of Pressure Sores or Pressure Ulcers

Pressure sores including diabetic ulcers, are areas of damaged skin and tissue that develop when sustained pressure (usually from a bed or wheelchair) cuts off circulation to vulnerable parts of the body, especially the skin on the buttocks, hips and heels. The lack of adequate blood flow leads to ischemic necrosis and ulceration of the affected tissue. Pressure sores occur most often in patients with diminished or absent sensation or who are debilitated, emaciated, paralyzed, or long bedridden. Tissues over the sacrum, ischia, greater trochanters, external malleoli, and heels are especially susceptible; other sites may be involved depending on the patient's situation.

Testing the active inhibitors of the invention (such as siRNA) for treating pressure sore, ulcers and similar wounds is performed in the mouse model described in Reid et al., Cyclical Magnetic Pressure Necrosis: A Novel Murine Model of Cutaneous Ischemia-Reperfusion Injury. J Surgical Research 2004, 116: 172-180.

Additionally, rabbit models described by Mustoe et al, JCI, 1991; Alm & Mustoe, Ann Pl Surg, 1991 and is used for testing the siRNAs of the invention.

### Example 4

### Model Systems of Chronic Obstructive Pulmonary Disease (COPD)

Chronic obstructive pulmonary disease (COPD) is characterized mainly by emphysema, which is permanent destruction of peripheral air spaces, distal to terminal bronchioles. Emphysema is also characterized by accumulation of inflammatory cells such as macrophages and neutrophils in bronchioles and alveolar structures. Emphysema and chronic bronchitis may occur as part of COPD or independently.

Testing the active inhibitors of the invention (such as siRNA) for treating COPD/emphysema/chronic bronchitis is done in the following animal models:

Cigarette smoke-induced emphysema model: chronic exposure to cigarette smoke causes emphysema in several animals such as, inter alia, mouse, guinea pig.
Lung protease activity as a trigger of emphysema.
VEGFR inhibition model of emphysema.
Bronchial instillation with human neutrophil/pancreatic elastase in rodents.
   MMP (matrix metalloprotease)-induced emphysema.
   Inflammation-induced emphysema.

These models are described in co-assigned PCT patent application WO 2006/023544, which is hereby incorporated by reference into this application. The siRNAs of the invention prevent formation of emphysema.

### Example 5

### Model Systems of Spinal Cord Injury

Spinal cord injury, or myelopathy, is a disturbance of the spinal cord that results in loss of sensation and/or mobility. The two common types of spinal cord injury are due to trauma and disease. Traumatic injury can be due to automobile accidents, falls, gunshot, diving accidents inter edict, and diseases which can affect the spinal cord include polio, spina bifida, tumors and Friedreich's ataxia.

Testing the active inhibitors of the invention (such as siRNA) for treating, spinal cord injury is done in the rat spinal cord contusion model as described by Young (Prog Brain Res. 2002; 137:231-55). Other predictive animal models of spinal cord injury are described in the following references:

Gruner J Neurotrauma. 1992. 9(2): 123; Hasegawa and Grumet J Neurosurg 2003. 98(5): 1065-71; and Huang and Young J Neurotrauma 1994, 11(5): 547.

### Example 6

### Model Systems of Glaucoma

Testing the active inhibitors of the invention (such as siRNA) for treating or preventing glaucoma is done in the animal model for example as described by Pease et al. J. Glaucoma 2006, 15(6):512-9.

### Example 7

### Model Systems of Ischemia Reperfusion Injury Following Lung Transplantation In Rats

Testing the active inhibitors of the invention (such as siRNA) for treating or preventing ischemia reperfusion (IR) injury following lung transplantation is performed in an animal model as described, for example, by Mizobuchi et al. J. Heart Lung Transplant 2004:23:889-93.

### Example 8

### Model Systems of Acute Respiratory Distress Syndrome

Testing the active inhibitors of the invention (such as siRNA) for treating Acute respiratory distress syndrome is performed in an animal model as described, for example, by Chen et al. J Biomed Sci. 2003; 10(6 Pt 1):588-92.

### Example 9

### Model Systems of Hearing Loss Conditions

(i) Distribution of Cy3-PTEN siRNA in the Cochlea Following Local Application to the Round Window of the Ear:

A solution of 1 μg/100 μl of Cy3-PTEN siRNA (total of 0.3-0.4 μg) PBS was applied to the round window of chinchillas. The Cy3-labelled cells within the treated cochlea were analyzed 24-48 hours post siRNA round window application after sacrifice of the chinchillas. The pattern of labeling within the cochlea was similar following 24 h and 48 h and includes labeling in the basal turn of cochlea, in the middle turn of cochlea and in the apical turn of cochlea. Application of Cy3-PTEN siRNA onto scala tympani revealed labelling mainly in the basal turn of the cochlea and the middle turn of the cochlea. The Cy3 signal is persistent to up to 15 days after the application of the Cy3-PTEN siRNA. These results indicate for the first time that local application of siRNA molecules within the round window leads to significant penetration of the siRNA molecules to the basal, middle and apical turns of the cochlea. The active siRNAs of the invention are tested in this model.

### (ii) Animal Model of Carboplatin-Induced or Cisplatin-Induced Hair Cell Death in the Cochlea of Chinchilla:

Chinchillas are pre-treated by direct administration of siRNA of the invention in saline to the left ear of each animal. Saline is given to the right ear of each animal as placebo. Two days following the administration of the specific siRNA, the animals are treated with carboplatin (75 mg/kg ip) or cisplatin (intraperitoneal infusion of 13 mg/kg over 30 minutes). After sacrifice of the chinchillas (two weeks post carboplatin treatment) the % of dead cells of inner hair cells (IHC) and outer hair cells (OHC) is calculated in the left ear (siRNA treated) and the right ear (saline treated). The results show that the siRNA prevents cell death of inner hair cells.

(iii) Animal Model of Acoustic-Induced Hair Cell Death in the Cochlea of Chinchilla:

The activity of specific siRNA to each NOX gene described herein is studied in an acoustic trauma model in chinchilla. The animals are exposed to an octave band of noise centered at 4 kHz for 2.5 h at 105 dB. The left ear of the noise-exposed chinchillas is pre-treated (48 h before the acoustic trauma) with 30 μg of siRNA in ˜10 μL of saline; the right ear is pre-treated with vehicle (saline). The compound action potential (CAP) is a convenient and reliable electrophysiological method for measuring the neural activity transmitted from the cochlea. The CAP is recorded by placing an electrode near the base of the cochlea in order to detect the local field potential that is generated when a sound stimulus, such as click or tone burst, is abruptly turned on. The functional status of each ear is assessed 2.5 weeks after the acoustic trauma. Specifically, the mean threshold of the compound action potential recorded from the round window is determined 2.5 weeks after the acoustic trauma in order to determine if the thresholds in the siRNA-treated ear are lower (better) than the untreated (saline) ear. In addition, the amount of inner and outer hair cell loss is determined in the siRNA-treated and the control ear.

The results show that in the siRNA-treated ear there is less cell death of inner and outer hair cells compared to the control ear.

### Example 10

### Generation of Sequences for Active siRNA Compounds to the NOX Genes and Production of the siRNAs

Using proprietary algorithms and the known sequence of each NOX gene, the sequences of potential siRNAs were generated. Tables A and B show NOX4 19-mer and 21-mer siRNAs, respectively, as 5′-3′ sequences, which are prioritized based on their score in the proprietary algorithm as the best sequences for targeting the human gene expression. A list of preferred siRNA to NOX1 is provided in tables C and D, infra. A list of preferred siRNA to NOX2 is provided in tables E and F, infra. A list of preferred siRNA to NOX5 is provided in tables G and H, infra. A list of preferred siRNA to DUOX2 is provided in tables I and J, infra. A list of preferred siRNA to NOXO1 is provided in tables K and L infra. A list of preferred siRNA to NOXO2 is provided in tables M and N, infra. A list of preferred siRNA to NOXA1 is provided in tables O and P H, infra. A list of preferred siRNA to NOXA2 (p67phox) is provided in tables Q and R, infra.

The siRNAs used in the experiments described herein are all 19-mers, having alternating ribonucleotides modified in both the antisense and the sense strands of the compound. The modification is such that a 2′-O-methyl (Me) group is present on the first, third, fifth, seventh, ninth, eleventh, thirteenth, fifteenth, seventeenth and nineteenth nucleotide of the antisense strand, whereby the very same modification, i.e. a 2′-O-Me group, is present at the second, fourth, sixth, eighth, tenth, twelfth, fourteenth, sixteenth and eighteenth nucleotide of the sense strand. These particular siRNA compounds are also blunt ended and are non-phosphorylated at the termini; however, comparative experiments have shown that siRNAs phosphorylated at the 3′-termini have similar activity to non-phosphorylated siRNAs in vivo.

TABLE A
19-mer oligomers for NOX4 (NADPH oxidase 4)

| | | | | |
|---|---|---|---|---|
| | | | | HUMAN-20149638 |
| NUMBER | SENSE SIRNA | ANTISENSE SIRNA | OTHER SP | ORF: 240-1976 |

| | | | | |
|---|---|---|---|---|
| 1 | GCCCUUCAUUCAAUCUAGA | UCUAGAUUGAAUGAAGGGC | Chimp, Rat | [1427-1445] ORF |
| 2 | CAUCCAUUUACCCUCACAA | UUGUGAGGGUAAAUGGAUG | Chimp | [1293-1311] ORF |
| 3 | UCAUCCAUUUACCCUCACA | UGUGAGGGUAAAUGGAUGA | Chimp | [1292-1310] ORF |
| 4 | GCCUUAUCCUUUUACCCAU | AUGGGUAAAAGGAUAAGGC | | [445-483] ORF |
| 5 | CAAUACUCAACACCCUGUU | AACAGGGUGUUGAGUAUUG | | [1558-1578] ORF |
| 6 | CCCUUCAUUCAAUCUAGAA | UUCUAGAUUGAAUGAAGGG | Chimp, Rat | [1428-1448] ORF |
| 7 | GGACUCUAAAGAAGGAAUG | CAUUCCUUCUUUAGAGUCC | Chimp | [1996-2014] 3′UTR |
| 8 | GGAAACUUUUGUUUGAUGA | UCAUCAAACAAAAGUUUCC | Chimp | [1818-1834] ORF |
| 9 | CCGUUGGUUUGCAGAUUUA | UAAAUCUGCAAACCAACGG | Chimp | [1548-1864] ORF |
| 10 | CUGCCCUUCAUUCAAUCUA | UAGAUUGAAUGAAGGGCAG | Chimp, Rat | [1425-1443] ORF |
| 11 | GAGUAUUUCUCAGAACAUU | AAUGUUCUGAGAAAUACUC | Chimp | [960-978] ORF |
| 12 | CCAUGAAUGUCCUGCUUUU | AAAAGCAGGACAUUCAUGG | Chimp | [310-328] ORF |
| 13 | CUGCCUCCAUCUAGUCAAG | CUUGACUAGAUGGAGGCAG | Chimp | [1395-1413] ORF |
| 14 | GGGUUCAUCUUAAAAUAGU | ACUAUUUUAAGAUGAACCC | Chimp | [1342-1360] ORF |
| 15 | CCAACUGAAACCAAAGCAA | UUGCUUUGGUUUCAGUUGG | Chimp | [1317-1335] ORF |
| 16 | CGAAAGACUUUACAGGUAU | AUACCUGUAAAGUCUUUCG | Chimp | [1127-1145] ORF |
| 17 | CCGAAAGACUUUACAGGUA | UACCUGUAAAGUCUUUCGG | Chimp | [1126-1144] ORF |
| 18 | GGAUUUUCAAAACCGGCAG | CUGCCGGUUUUGAAAAUCC | Chimp | [999-1017] ORF |
| 19 | GGAGAUUGUUGGAUAAAAG | CUUUUAUCCAACAAUCUCC | Chimp | [523-541] ORF |
| 20 | GCACACUGUUGAUUUUCAU | AUGAAAAUCAACAGUGUGC | | [2237-2255] 3′UTR |
| 21 | GGAGAAAAAUAUCAUGCAC | GUGCAUGAUAUUUUUCUCC | Chimp | [1764-1782] ORF |
| 22 | UGCCCUUCAUUCAAUCUAG | CUAGAUUGAAUGAAGGGCA | Chimp, Rat | [1426-1444] ORF |
| 23 | UGCUAUUCCUCAUGAUCAC | GUGAUCAUGAGGAAUAGCA | Chimp | [748-768] ORF |
| 24 | GUGGAAACUUUUGUUUGAU | AUCAAACAAAAGUUUCCAC | Chimp | [1814-1832] ORF |
| 25 | UCAAUACUCAACACCCUGU | ACAGGGUGUUGAGUAUUGA | | [1557-1575] ORF |
| 26 | GUAUAUUGAUGGUCCUUUU | AAAAGGACCAUCAAUAUAC | Chimp | [1460-1478] ORF |
| 27 | ACUGCCUCCAUCUAGUCAA | UUGACUAGAUGGAGGCAGU | Chimp | [1394-1412] ORF |
| 28 | GGAGACUGGACAGAACGAU | AUCGUUCUGUCCAGUCUCC | Chimp, Ms | [1362-1380] ORF |
| 29 | CACAAUGUGUCCAACUGAA | UUCAGUUGGACACAUUGUG | Chimp | [1307-1325] ORF |
| 30 | CCGGCUGCAUCAGUCUUAA | UUAAGACUGAUGCAGCCGG | Chimp | [910-928] ORF |
| 31 | CUAUUCCUCAUGAUCACAG | CUGUGAUCAUGAGGAAUAG | Chimp | [750-768] ORF |
| 32 | UCACAACUGUUCCUGGCCU | AGGCCAGGAACAGUUGUGA | Chimp | [706-724] ORF |
| 33 | UGAGGAUCCUAGAAAACUU | AAGUUUUCUAGGAUCCUCA | Chimp | [683-701] ORF |
| 34 | CAACUUCUCAGUGAAUUAC | GUAAUUCACUGAGAAGUUG | Chimp | [623-641] ORF |
| 35 | AGGAGAUUGUUGGAUAAAA | UUUUAUCCAACAAUCUCCU | Chimp | [522-540] ORF |
| 36 | CCUUAUCCUUUUACCCAUG | CAUGGGUAAAAGGAUAAGG | | [446-464] ORF |
| 37 | AGGACUCUAAAGAAGGAAU | AUUCCUUCUUUAGAGUCCU | Chimp | [1995-2013] 3′UTR |
| 38 | CCCAAUUCACUAUCCAAGA | UCUUGGAUAGUGAAUUGGG | Chimp | [1884-1902] ORF |
| 39 | GACCCAAUUCACUAUCCAA | UUGGAUAGUGAAUUGGGUC | Chimp | [1882-1900] ORF |
| 40 | GCAUAACAAGUUUUGGCAA | UUGCCAAAACUUGUUAUGC | Chimp, Rat, Ms | [1676-1694] ORF |
| 41 | GGCUAGGAUUGUGUCUAAG | CUUAGACACAAUCCUAGCC | Chimp | [391-409] ORF |
| 42 | CAGAACGAUUUCGAGAUUU | AAAUCUCGAAAUCGUUCUG | Chimp | [1372-1390] ORF |
| 43 | CGAAUGGUCAAAGAAAAUU | AAUUUUCUUUGACCAUUCG | Chimp | [1212-1230] ORF |
| 44 | CUCAUGAUCACAGCCUCUA | UAGAGGCUGUGAUCAUGAG | Chimp | [756-774] ORF |
| 45 | UGGUGUUACUAUCUGUAUU | AAUACAGAUAGUAACACCA | Chimp | [563-581] ORF |
| 46 | GGUUGAACAAAUGCAAGAU | AUCUUGCAUUUGUUCAACC | Chimp | [2338-2356] 3′UTR |
| 47 | CUAGUGAGGAGCUGAACUU | AAGUUCAGCUCCUCACUAG | Chimp | [2274-2292] 3′UTR |
| 48 | GGAAUCAAUCAGCUGUGUU | AACACAGCUGAUUGAUUCC | Chimp | [2045-2083] 3′UTR |
| 49 | CGGAAUCAAUCAGCUGUGU | ACACAGCUGAUUGAUUCCG | Chimp | [2044-2062] 3′UTR |
| 50 | GCAAUUUCUAAGACUUUGA | UCAAAGUCUUAGAAAUUGC | Chimp | [2018-2036] 3′UTR |
| 51 | ACCCAAUUCACUAUCCAAG | CUUGGAUAGUGAAUUGGGU | Chimp | [1883-1901] ORF |
| 52 | AGGAAAAACAGUUGGUGUU | AACACCAACUGUUUUUCCU | | [1853-1871] ORF |
| 53 | UGGAAACUUUUGUUUGAUG | CAUCAAACAAAAGUUUCCA | Chimp | [1815-1833] ORF |
| 54 | CUGAAUUCAAGACUGUUUA | UAAACAGUCUUGAAUUCAG | Chimp | [1782-1800] ORF |
| 55 | CAGAAGAUAAUUGGAGAAA | UUUCUCCAAUUAUCUUCUG | Chimp, Rat, Ms | [1752-1770] ORF |
| 56 | GUAACUCCAUUUGCAUCAA | UUGAUGCAAAUGGAGUUAC | Chimp | [1542-1560] ORF |
| 57 | AGAAAAUCAUCCAUUUACC | GGUAAAUGGAUGAUUUUCU | Chimp | [1286-1304] ORF |
| 58 | GAAAGACUUUACAGGUAUA | UAUACCUGUAAAGUCUUUC | Chimp | [1128-1146] ORF |
| 59 | GAAAACUUCUCUUCACAAC | GUUGUGAAGAGAAGUUUUC | Chimp | [694-712] ORF |
| 60 | GGAUCCUAGAAAACUUCUC | GAGAAGUUUUCUAGGAUCC | Chimp | [686-704] ORF |
| 61 | AGACUUUGUUGAACUGAAU | AUUCAGUUCAACAAAGUCU | | [647-685] ORF |
| 62 | GUUGAACAAAUGCAAGAUU | AAUCUUGCAUUUGUUCAAC | Chimp | [2339-2357] 3′UTR |
| 63 | CUUAUCCUUUUACCCAUGU | ACAUGGGUAAAAGGAUAAG | | [447-465] ORF |
| 64 | CUGCAGCCUUAUCCUUUUA | UAAAAGGAUAAGGCUGCAG | Chimp | [440-458] ORF |
| 65 | UGCCAAAGAAUAGUAAGGU | ACCUUACUAUUCUUUGGCA | Chimp | [2065-2083] 3′UTR |
| 66 | AGAAGGAAUGAGUGCAAUU | AAUUGCACUCAUUCCUUCU | Chimp | [2005-2023] 3′UTR |
| 67 | AAAGAAGGAAUGAGUGCAA | UUGCACUCAUUCCUUCUUU | Chimp | [2003-2021] 3′UTR |
| 68 | CUCUAAAGAAGGAAUGAGU | ACUCAUUCCUUCUUUAGAG | Chimp | [1999-2017] 3′UTR |
| 69 | CAGGACUCUAAAGAAGGAA | UUCCUUCUUUAGAGUCCUG | Chimp | [1994-2012] 3′UTR |
| 70 | CCAAGACUCUUCAUAAACU | AGUUUAUGAAGAGUCUUGG | Chimp | [1897-1915] ORF |
| 71 | CUGUUCUUAACCUCAACUG | CAGUUGAGGUUAAGAACAG | Chimp | [424-442] ORF |
| 72 | CAAUUCACUAUCCAAGACU | AGUCUUGGAUAGUGAAUUG | Chimp | [1886-1904] ORF |
| 73 | CAUCUGUUCUUAACCUCAA | UUGAGGUUAAGAACAGAUG | Chimp | [421-439] ORF |
| 74 | GCAAAAUAUAACAGAGGAA | UUCCUCUGUUAUAUUUUGC | | [1839-1857] ORF |
| 75 | GGUGGAAACUUUUGUUUGA | UCAAACAAAAGUUUCCACC | Chimp | [1813-1831] ORF |
| 76 | UCGGUGGAAACUUUUGUUU | AAACAAAAGUUUCCACCGA | Chimp | [1811-1829] ORF |
| 77 | AGAAAAAUAUCAUGCACUG | CAGUGCAUGAUAUUUUUCU | Chimp | [1766-1784] ORF |
| 78 | ACAGAAGAUAAUUGGAGAA | UUCUCCAAUUAUCUUCUGU | Chimp, Rat | [1751-1769] ORF |
| 79 | CUAGGAUUGUGUCUAAGCA | UGCUUAGACACAAUCCUAG | Chimp | [393-411] ORF |
| 80 | CCCAAGCUGUAUAUUGAUG | CAUCAAUAUACAGCUUGGG | Chimp | [1452-1470] ORF |
| 81 | ACGAUUUCGAGAUUUACUA | UAGUAAAUCUCGAAAUCGU | Chimp | [1376-1394] ORF |
| 82 | GACAGAACGAUUUCGAGAU | AUCUCGAAAUCGUUCUGUC | Chimp | [1370-1388] ORF |
| 83 | ACAAUGUGUCCAACUGAAA | UUUCAGUUGGACACAUUGU | Chimp | [1308-1326] ORF |
| 84 | ACCCUCACAAUGUGUCCAA | UUGGACACAUUGUGAGGGU | Chimp | [1302-1320] ORF |
| 85 | CCUGUACUGUGCCGAAAGA | UCUUUCGGCACAGUACAGG | Chimp | [1115-1133] ORF |
| 86 | CAGAUUCCAAGCUAAUUUU | AAAAUUAGCUUGGAAUCUG | Chimp | [1061-1079] ORF |
| 87 | CAAACUAAUUUAGAUACCC | GGGUAUCUAAAUUAGUUUG | Chimp | [885-903] ORF |
| 88 | AGAUGAGGAUCCUAGAAAA | UUUUCUAGGAUCCUCAUCU | Chimp | [680-698] ORF |
| 89 | CGAGAUGAGGAUCCUAGAA | UUCUAGGAUCCUCAUCUCG | Chimp | [678-696] ORF |
| 90 | UGAAUUACAGUGAAGACUU | AAGUCUUCACUGUAAUUCA | | [634-652] ORF |
| 91 | UCGUGUUCCUCUUUAAAUU | AAUUUAAAGAGGAACACGA | Chimp | [2313-2331] 3′UTR |
| 92 | GAGACCCUGAAGAAUAUUU | AAAUAUUCUUCAGGGUCUC | Chimp | [2168-2186] 3′UTR |
| 93 | ACAUGUUUAAUCUGGAAAC | GUUUCCAGAUUAAACAUGU | Chimp | [2145-2163] 3′UTR |
| 94 | GCAGCCUUAUCCUUUUACC | GGUAAAAGGAUAAGGCUGC | Chimp | [442-460] ORF |
| 95 | GUUAUGCCAAAGAAUAGUA | UACUAUUCUUUGGCAUAAC | Chimp | [2061-2079] 3′UTR |
| 96 | UGAGUGCAAUUUCUAAGAC | GUCUUAGAAAUUGCACUCA | Chimp | [2013-2031] 3′UTR |
| 97 | ACAAUAAAGAGUCUUUCAG | CUGAAAGACUCUUUAUUGU | Chimp | [1954-1972] ORF |
| 98 | ACAACUCAUAUGGGACAAG | CUUGUCCCAUAUGAGUUGU | | [1927-1945] ORF |
| 99 | GUGGACCCAAUUCACUAUC | GAUAGUGAAUUGGGUCCAC | Chimp | [1879-1897] ORF |
| 100 | CUCGGUGGAAACUUUUGUU | AACAAAAGUUUCCACCGAG | Chimp | [1810-1828] ORF |
| 101 | GAGAAAAAUAUCAUGCACU | AGUGCAUGAUAUUUUUCUC | Chimp | [1765-1783] ORF |
| 102 | UGGAGAAAAAUAUCAUGCA | UGCAUGAUAUUUUUCUCCA | Chimp | [1763-1781] ORF |
| 103 | ACAAGUUUUGGCAAGAGAA | UUCUCUUGCCAAAACUUGU | Chimp | [1681-1699] ORF |
| 104 | UGAGGAAUCACUGAACUAU | AUAGUUCAGUGAUUCCUCA | Chimp | [1490-1508] ORF |
| 105 | GCUGUAUAUUGAUGGUCCU | AGGACCAUCAAUAUACAGC | Chimp | [1457-1475] ORF |
| 106 | CAAGCUGUAUAUUGAUGGU | ACCAUCAAUAUACAGCUUG | Chimp | [1454-1472] ORF |
| 107 | UGGUCAGUAUAUUACUCUA | UAGAGUAAUAUACUGACCA | Chimp | [1244-1282] ORF |
| 108 | CCGAAUGGUCAAAGAAAAU | AUUUUCUUUGACCAUUCGG | Chimp | [1211-1229] ORF |
| 109 | AGUUUACCCAGCACAAAUU | AAUUUGUGCUGGGUAAACU | Chimp | [1018-1036] ORF |
| 110 | GAGUUUACCCAGCACAAAU | AUUUGUGCUGGGUAAACUC | Chimp | [1017-1035] ORF |
| 111 | UCCUCAUGAUCACAGCCUC | GAGGCUGUGAUCAUGAGGA | Chimp | [754-772] ORF |
| 112 | CUCCAUGAAUGUCCUGCUU | AAGCAGGACAUUCAUGGAG | Chimp | [308-328] ORF |
| 113 | GUGAAUUACAGUGAAGACU | AGUCUUCACUGUAAUUCAC | | [633-651] ORF |
| 114 | CAGUGAAUUACAGUGAAGA | UCUUCACUGUAAUUCACUG | | [631-649] ORF |
| 115 | CCGAGGAUCACAGAAGGUU | AACCUUCUGUGAUCCUCGG | Chimp | [488-506] ORF |
| 116 | CCGAACACUCUUGGCUUAC | GUAAGCCAAGAGUGUUCGG | Chimp | [467-485] ORF |
| 117 | CUUUUACCCAUGUGCCGAA | UUCGGCACAUGGGUAAAAG | | [453-471] ORF |
| 118 | UGAUUUUCAUGGUGGAUUC | GAAUCCACCAUGAAAAUCA | | [2246-2264] 3′UTR |
| 119 | AGGAAUGAGUGCAAUUUCU | AGAAAUUGCACUCAUUCCU | Chimp | [2008-2028] 3′UTR |
| 120 | AAGAAGGAAUGAGUGCAAU | AUUGCACUCAUUCCUUCUU | Chimp | [2004-2022] 3′UTR |
| 121 | UGGGACAAGAUUUGAAUAC | GUAUUCAAAUCUUGUCCCA | | [1937-1955] ORF |
| 122 | GCAUCUGUUCUUAACCUCA | UGAGGUUAAGAACAGAUGC | Chimp | [420-438] ORF |
| 123 | ACUGAAUUCAAGACUGUUU | AAACAGUCUUGAAUUCAGU | Chimp | [1781-1799] ORF |
| 124 | GGGAUACAGAAGAUAAUUG | CAAUUAUCUUCUGUAUCCC | Chimp, Rat | [1746-1764] ORF |
| 125 | CUCUGUAUGUUGCAUAACA | UGUUAUGCAACAUACAGAG | Chimp | [1665-1683] ORF |
| 126 | UGCAGAGAUAUCCAGUCCU | AGGACUGGAUAUCUCUGCA | Chimp | [1626-1644] ORF |
| 127 | GACUAUACUUUAUUUGGGU | ACCCAAAUAAAGUAUAGUC | Chimp | [1606-1624] ORF |
| 128 | CCUGUUGGAUGACUGGAAA | UUUCCAGUCAUCCAACAGG | | [1571-1589] ORF |
| 129 | GGAUUGUGUCUAAGCAGAG | CUCUGCUUAGACACAAUCC | Chimp | [395-414] ORF |
| 130 | AGGAUUGUGUCUAAGCAGA | UCUGCUUAGACACAAUCCU | Chimp | [395-413] ORF |
| 131 | AACUCCAUUUGCAUCAAUA | UAUUGAUGCAAAUGGAGUU | Chimp | [1544-1562] ORF |
| 132 | CCAAGCUGUAUAUUGAUGG | CCAUCAAUAUACAGCUUGG | Chimp | [1453-1471] ORF |
| 133 | ACUCCGAAAUUCUGCCCUU | AAGGGCAGAAUUUCGGAGU | Chimp | [1414-1432] ORF |
| 134 | CUGGACAGAACGAUUUCGA | UCGAAAUCGUUCUGUCCAG | Chimp | [1367-1385] ORF |
| 135 | GCCGAAAGACUUUACAGGU | ACCUGUAAAGUCUUUCGGC | Chimp | [1125-1143] ORF |
| 136 | CGGCUGCAUCAGUCUUAAC | GUUAAGACUGAUGCAGCCG | Chimp | [911-929] ORF |
| 137 | UCUACAUGCUGCUGACGUU | AACGUCAGCAGCAUGUAGA | Chimp | [838-856] ORF |
| 138 | UCUUCUGGUAUACUCAUAA | UUAUGAGUAUACCAGAAGA | Chimp | [805-823] ORF |
| 139 | GUGGUGUUACUAUCUGUAU | AUACAGAUAGUAACACCAC | Chimp | [562-580] ORF |
| 140 | CCUGUGGUGUUACUAUCUG | CAGAUAGUAACACCACAGG | Chimp | [559-577] ORF |
| 141 | CAGGAGAUUGUUGGAUAAA | UUUAUCCAACAAUCUCCUG | Chimp | [521-539] ORF |
| 142 | UGGUUGAACAAAUGCAAGA | UCUUGCAUUUGUUCAACCA | Chimp | [2337-2355] 3′UTR |
| 143 | CCCUGAAGAAUAUUUGAUG | CAUCAAAUAUUCUUCAGGG | Chimp, Rat | [2172-2190] 3′UTR |
| 144 | ACCCUGAAGAAUAUUUGAU | AUCAAAUAUUCUUCAGGGU | Chimp | [2171-2189] 3′UTR |
| 145 | AGAGACCCUGAAGAAUAUU | AAUAUUCUUCAGGGUCUCU | Chimp | [2167-2185] 3′UTR |
| 146 | CUGUGUUAUGCCAAAGAAU | AUUCUUUGGCAUAACACAG | Chimp | [2057-2075] 3′UTR |
| 147 | GAGUGCAAUUUCUAAGACU | AGUCUUAGAAAUUGCACUC | Chimp | [2014-2032] 3′UTR |
| 148 | UGAAAACUUUUGCCAUGAA | UUCAUGGCAAAAGUUUUCA | Chimp | [1974-1992] ORF + 3′UTR |
| 149 | GAACAACUCAUAUGGGACA | UGUCCCAUAUGAGUUGUUC | Rat, Ms | [1925-1943] ORF |
| 150 | AGAGGAAAAACAGUUGGUG | CACCAACUGUUUUUCCUCU | | [1851-1869] ORF |
| 151 | CGGUGGAAACUUUUGUUUG | CAAACAAAAGUUUCCACCG | Chimp | [1812-1830] ORF |
| 152 | CAGCAUCUGUUCUUAACCU | AGGUUAAGAACAGAUGCUG | Chimp | [418-436] ORF |
| 153 | UAACAAGUUUUGGCAAGAG | CUCUUGCCAAAACUUGUUA | Chimp | [1679-1697] ORF |
| 154 | UGCAUAACAAGUUUUGGCA | UGCCAAAACUUGUUAUGCA | Chimp, Rat, Ms | [1675-1893] ORF |
| 155 | UUACUCUGUAUGUUGCAUA | UAUGCAACAUACAGAGUAA | Chimp | [1662-1680] ORF |
| 156 | UGUAUAUUGAUGGUCCUUU | AAAGGACCAUCAAUAUACA | Chimp | [1459-1477] ORF |
| 157 | CGAUUUCGAGAUUUACUAC | GUAGUAAAUCUCGAAAUCG | Chimp | [1377-1395] ORF |
| 158 | GGACAGAACGAUUUCGAGA | UCUCGAAAUCGUUCUGUCC | Chimp | [1369-1387] ORF |
| 159 | CCCUCAGAUGUCAUGGAAA | UUUCCAUGACAUCUGAGGG | Chimp | [1191-1209] ORF |
| 160 | CCUUCUUGCUGUAUAACCA | UGGUUAUACAGCAAGAAGG | Chimp | [337-355] ORF |
| 161 | CAUGAACCUUUCCCUGAAG | CUUCAGGGAAAGGUUCAUG | Chimp | [961-999] ORF |
| 162 | CCAGAGUAUUUCUCAGAAC | GUUCUGAGAAAUACUCUGG | Chimp | [957-975] ORF |
| 163 | CCAGCUCUCAGAAUAUUUC | GAAAUAUUCUGAGAGCUGG | Chimp | [934-952] ORF |
| 164 | GACGUUGCAUGUUUCAGGA | UCCUGAAACAUGCAACGUC | Chimp | [851-869] ORF |
| 165 | CUAUGAUAUCUUCUGGUAU | AUACCAGAAGAUAUCAUAG | Chimp | [797-815] ORF |
| 166 | UGAAUGUCCUGCUUUUCUG | CAGAAAAGCAGGACAUUCA | Chimp | [313-331] ORF |
| 167 | CUAACUAUGAUAUCUUCUG | CAGAAGAUAUCAUAGUUAG | Chimp | [793-811] ORF |
| 168 | GCAAUAAGAGUUUCUAACU | AGUUAGAAACUCUUAUUGC | Chimp | [780-796] ORF |
| 169 | UGAUCACAGCCUCUACAUA | UAUGUAGAGGCUGUGAUCA | Chimp | [760-778] ORF |
| 170 | GAGGAUCCUAGAAAACUUC | GAAGUUUUCUAGGAUCCUC | Chimp | [684-702] ORF |
| 171 | UUACAGUGAAGACUUUGUU | AACAAAGUCUUCACUGUAA | | [638-658] ORF |
| 172 | ACAUUCCAUAUUACCUGUG | CACAGGUAAUAUGGAAUGU | Chimp | [546-564] ORF |
| 173 | GAACAUUCCAUAUUACCUG | CAGGUAAUAUGGAAUGUUC | Chimp | [544-562] ORF |
| 174 | GUGUUCCUCUUUAAAUUGU | ACAAUUUAAAGAGGAACAC | Chimp | [2315-2333] 3′UTR |
| 175 | GUCGUGUUCCUCUUUAAAU | AUUUAAAGAGGAACACGAC | Chimp | [2312-2330] 3′UTR |
| 176 | GGCUGAUUGUCGUGUUCCU | AGGAACACGACAAUCAGCC | Chimp | [2304-2322] 3′UTR |
| 177 | CACUGUUGAUUUUCAUGGU | ACCAUGAAAAUCAACAGUG | | [2240-2258] 3′UTR |
| 178 | UGCACACUGUUGAUUUUCA | UGAAAAUCAACAGUGUGCA | | [2236-2254] 3′UTR |
| 179 | AGACCCUGAAGAAUAUUUG | CAAAUAUUCUUCAGGGUCU | Chimp | [2169-2187] 3′UTR |
| 180 | GCAAGAUGACCGUCACAUU | AAUGUGACGGUCAUCUUGC | | [2126-2144] 3′UTR |
| 181 | UGCAGCCUUAUCCUUUUAC | GUAAAAGGAUAAGGCUGCA | Chimp | [441-459] ORF |
| 182 | ACUGCAGCCUUAUCCUUUU | AAAAGGAUAAGGCUGCAGU | Chimp | [439-457] ORF |
| 183 | GUGCAAUUUCUAAGACUUU | AAAGUCUUAGAAAUUGCAC | Chimp | [2016-2034] 3′UTR |
| 184 | AGUGCAAUUUCUAAGACUU | AAGUCUUAGAAAUUGCACU | Chimp, Rat, Ms | [2015-2033] 3′UTR |
| 185 | UGAAGCAGGACUCUAAAGA | UCUUUAGAGUCCUGCUUCA | Chimp | [1989-2007] 3′UTR |
| 186 | AAAGAGUCUUUCAGCUGAA | UUCAGCUGAAAGACUCUUU | Chimp | [1959-1977] ORF + 3′UTR |
| 187 | UCAUAUGGGACAAGAUUUG | CAAAUCUUGUCCCAUAUGA | | [1932-1950] ORF |
| 188 | AACAACUCAUAUGGGACAA | UUGUCCCAUAUGAGUUGUU | Rat, Ms | [1926-1944] ORF |
| 189 | CUAUCCAAGACUCUUCAUA | UAUGAAGAGUCUUGGAUAG | Chimp | [1893-1911] ORF |
| 190 | UCACUAUCCAAGACUCUUC | GAAGAGUCUUGGAUAGUGA | Chimp | [1890-1908] ORF |
| 191 | CAGAGGAAAAACAGUUGGU | ACCAACUGUUUUUCCUCUG | | [1850-1888] ORF |
| 192 | AGCAUCUGUUCUUAACCUC | GAGGUUAAGAACAGAUGCU | Chimp | [419-437] ORF |
| 193 | CCUCGGUGGAAACUUUUGU | ACAAAAGUUUCCACCGAGG | Chimp | [1809-1827] ORF |
| 194 | AGAUGGGAUACAGAAGAUA | UAUCUUCUGUAUCCCAUCU | Chimp, Rat | [1742-1760] ORF |
| 195 | UACUCUGUAUGUUGCAUAA | UUAUGCAACAUACAGAGUA | Chimp | [1683-1681] ORF |
| 196 | AGACUAUACUUUAUUUGGG | CCCAAAUAAAGUAUAGUCU | Chimp | [1605-1623] ORF |
| 197 | GCUGGAGGCAUUGGAGUAA | UUACUCCAAUGCCUCCAGC | Chimp | [1527-1545] ORF |
| 198 | UCCCAAGCUGUAUAUUGAU | AUCAAUAUACAGCUUGGGA | Chimp | [1451-1469] ORF |
| 199 | UCCAACUGAAACCAAAGCA | UGCUUUGGUUUCAGUUGGA | Chimp | [1316-1334] ORF |
| 200 | CUGGUCAGUAUAUUACUCU | AGAGUAAUAUACUGACCAG | Chimp | [1243-1261] ORF |
| 201 | CCAUCAUUUCGGUCAUAAG | CUUAUGACCGAAAUGAUGG | Chimp | [1168-1186] ORF |
| 202 | GGUAUAUCCGGAGCAAUAA | UUAUUGCUCCGGAUAUACC | Chimp | [1141-1159] ORF |
| 203 | GGAUUUCUGGACCUUUGUG | CACAAAGGUCCAGAAAUCC | Chimp, Rat, Ms | [1096-1114] ORF |
| 204 | AAAACCUUCUUGCUGUAUA | UAUACAGCAAGAAGGUUUU | Chimp | [333-351] ORF |
| 205 | AUACUCAUAACCUCUUCUU | AAGAAGAGGUUAUGAGUAU | Chimp | [814-832] ORF |
| 206 | GUAUACUCAUAACCUCUUC | GAAGAGGUUAUGAGUAUAC | Chimp | [812-830] ORF |
| 207 | CUUCUGGUAUACUCAUAAC | GUUAUGAGUAUACCAGAAG | Chimp | [806-824] ORF |
| 208 | AUCUUCUGGUAUACUCAUA | UAUGAGUAUACCAGAAGAU | Chimp | [804-822] ORF |
| 209 | UCCAUGAAUGUCCUGCUUU | AAAGCAGGACAUUCAUGGA | Chimp | [309-327] ORF |
| 210 | GUGAAGACUUUGUUGAACU | AGUUCAACAAAGUCUUCAC | | [643-661] ORF |
| 211 | ACAGUGAAGACUUUGUUGA | UCAACAAAGUCUUCACUGU | | [640-658] ORF |
| 212 | ACAAAUGCAAGAUUGAACA | UGUUCAAUCUUGCAUUUGU | Chimp | [2344-2362] 3′UTR |
| 213 | UUGAUUUUCAUGGUGGAUU | AAUCCACCAUGAAAAUCAA | | [2245-2263] 3′UTR |
| 214 | AGAUGCACACUGUUGAUUU | AAAUCAACAGUGUGCAUCU | Chimp, Rat | [2233-2251] 3′UTR |
| 215 | ACCUCAACUGCAGCCUUAU | AUAAGGCUGCAGUUGAGGU | Chimp | [433-451] ORF |
| 216 | CUCAUAUGGGACAAGAUUU | AAAUCUUGUCCCAUAUGAG | | [1931-1949] ORF |
| 217 | CAGAACAACUCAUAUGGGA | UCCCAUAUGAGUUGUUCUG | | [1923-1941] ORF |
| 218 | CCAGAACAACUCAUAUGGG | CCCAUAUGAGUUGUUCUGG | | [1922-1940] ORF |
| 219 | CAAGACUCUUCAUAAACUG | CAGUUUAUGAAGAGUCUUG | Chimp | [1898-1916] ORF |
| 220 | GGUGUUUUCUGUUGUGGAC | GUCCACAACAGAAAACACC | Chimp | [1868-1884] ORF |
| 221 | CAGUUGGUGUUUUCUGUUG | CAACAGAAAACACCAACUG | Chimp | [1861-1879] ORF |
| 222 | UCAAGACUGUUUAUAGGAC | GUCCUAUAAACAGUCUUGA | | [1788-1808] ORF |
| 223 | CACUGAAUUCAAGACUGUU | AACAGUCUUGAAUUCAGUG | Chimp | [1780-1798] ORF |
| 224 | GGAUACAGAAGAUAAUUGG | CCAAUUAUCUUCUGUAUCC | Chimp, Rat | [1747-1765] ORF |
| 225 | AUGGGAUACAGAAGAUAAU | AUUAUGUUCUGUAUCCCAU | Chimp, Rat | [1744-1762] ORF |
| 226 | AGAUUUACUCUGUAUGUUG | CAACAUACAGAGUAAAUCU | Chimp | [1658-1676] ORF |
| 227 | CUGUAUAUUGAUGGUCCUU | AAGGACCAUCAAUAUACAG | Chimp | [1458-1476] ORF |
| 228 | UCGAGAUUUACUACUGCCU | AGGCAGUAGUAAAUCUCGA | Chimp | [1382-1400] ORF |
| 229 | GUAUCUGCAUUAGAAAAUC | GAUUUUCUAAUGCAGAUAC | Chimp | [1275-1293] ORF |
| 230 | CCUGGUCAGUAUAUUACUC | GAGUAAUAUACUGACCAGG | Chimp | [1242-1260] ORF |
| 231 | GACUUGGCUUUGGAUUUCU | AGAAAUCCAAAGCCAAGUC | Chimp | [1085-1103] ORF |
| 232 | UGAUAUCUUCUGGUAUACU | AGUAUACCAGAAGAUAUCA | Chimp, Rat, Ms | [800-818] ORF |
| 233 | AAGACUUUGUUGAACUGAA | UUCAGUUCAACAAAGUCUU | | [646-664] ORF |
| 234 | UGAAGACUUUGUUGAACUG | CAGUUCAACAAAGUCUUCA | | [644-662] ORF |
| 235 | CAGUGAAGACUUUGUUGAA | UUCAACAAAGUCUUCACUG | | [641-659] ORF |
| 236 | UGUGGUGUUACUAUCUGUA | UACAGAUAGUAACACCACA | Chimp | [581-579] ORF |
| 237 | CUGUGGUGUUACUAUCUGU | ACAGAUAGUAACACCACAG | Chimp | [580-578] ORF |
| 238 | UGAACAAAUGCAAGAUUGA | UCAAUCUUGCAUUUGUUCA | Chimp | [2341-2359] 3′UTR |
| 239 | UUGUCGUGUUCCUCUUUAA | UUAAAGAGGAACACGACAA | Chimp | [2310-2328] 3′UTR |
| 240 | UGAUUGUCGUGUUCCUCUU | AAGAGGAACACGACAAUCA | Chimp | [2307-2325] 3′UTR |
| 241 | ACUGUUGAUUUUCAUGGUG | CACCAUGAAAAUCAACAGU | | [2241-2259] 3′UTR |
| 242 | CCUGAAGAAUAUUUGAUGU | ACAUCAAAUAUUCUUCAGG | Chimp, Rat | [2173-2191] 3′UTR |
| 243 | GGAAAUGUGAGAAUGUGGC | GCCACAUUCUCACAUUUCC | Chimp | [2109-2127] 3′UTR |
| 244 | CAACUGCAGCCUUAUCCUU | AAGGAUAAGGCUGCAGUUG | Chimp | [437-455] ORF |
| 245 | UGCAAUUUCUAAGACUUUG | CAAAGUCUUAGAAAUUGCA | Chimp | [2017-2035] 3′UTR |
| 246 | GCUGAAAACUUUUGCCAUG | CAUGGCAAAAGUUUUCAGC | Chimp | [1972-1990] ORF + 3′UTR |
| 247 | AUAUGGGACAAGAUUUGAA | UUCAAAUCUUGUCCCAUAU | | [1934-1952] ORF |
| 248 | AUCCAAGACUCUUCAUAAA | UUUAUGAAGAGUCUUGGAU | Chimp | [1895-1913] ORF |
| 249 | CACUAUCCAAGACUCUUCA | UGAAGAGUCUUGGAUAGUG | Chimp | [1891-1909] ORF |
| 250 | UAACAGAGGAAAAACAGUU | AACUGUUUUUCCUCUGUUA | | [1847-1865] ORF |
| 251 | UGGGAUACAGAAGAUAAUU | AAUUAUCUUCUGUAUCCCA | Chimp, Rat | [1745-1763] ORF |
| 252 | CAGUCAAACAGAUGGGAUA | UAUCCCAUCUGUUUGACUG | Chimp, Rat | [1733-1751] ORF |
| 253 | GGGUAUGCAGAGAUAUCCA | UGGAUAUCUCUGCAUACCC | Chimp | [1621-1639] ORF |
| 254 | UGGAGGCAUUGGAGUAACU | AGUUACUCCAAUGCCUCCA | Chimp | [1529-1547] ORF |
| 255 | CCAUUUGAGGAAUCACUGA | UCAGUGAUUCCUCAAAUGG | Chimp | [1485-1503] ORF |
| 256 | GACCUGGUCAGUAUAUUAC | GUAAUAUACUGACCAGGUC | Chimp | [1240-1258] ORF |
| 257 | GGAAAUCCGAAUGGUCAAA | UUUGACCAUUCGGAUUUCC | Chimp | [1205-1223] ORF |
| 258 | UCACCAUCAUUUCGGUCAU | AUGACCGAAAUGAUGGUGA | Chimp | [1165-1183] ORF |
| 259 | CAGACUUGGCUUUGGAUUU | AAAUCCAAAGCCAAGUCUG | Chimp | [1083-1101] ORF |
| 260 | ACAGACUUGGCUUUGGAUU | AAUCCAAAGCCAAGUCUGU | Chimp | [1082-1100] ORF |
| 261 | GCUAAUUUUCCACAGACUU | AAGUCUGUGGAAAAUUAGC | Chimp | [1071-1089] ORF |
| 262 | UGUGAAGAUUUGUAUGGAA | UUCCAUACAAAUCUUCACA | Chimp | [1037-1055] ORF |
| 263 | GAAAACCUUCUUGCUGUAU | AUACAGCAAGAAGGUUUUC | Chimp | [332-350] ORF |
| 264 | CUCAUAACCUCUUCUUUGU | ACAAAGAAGAGGUUAUGAG | Chimp | [817-835] ORF |
| 265 | UGCAAUAAGAGUUUCUAAC | GUUAGAAACUCUUAUUGCA | Chimp | [779-797] ORF |
| 266 | AAAAGCAGAACAUUCCAUA | UAUGGAAUGUUCUGCUUUU | Chimp | [537-555] ORF |
| 267 | UAAAAGCAGAACAUUCCAU | AUGGAAUGUUCUGCUUUUA | Chimp | [536-554] ORF |
| 268 | CGUGUUCCUCUUUAAAUUG | CAAUUUAAAGAGGAACACG | Chimp | [2314-2332] 3′UTR |
| 269 | UAAGGCUGAUUGUCGUGUU | AACACGACAAUCAGCCUUA | Chimp | [2301-2319] 3′UTR |
| 270 | UCAUGGUGGAUUCAAGAAC | GUUCUUGAAUCCACCAUGA | | [2252-2270] 3′UTR |
| 271 | CUGAAGAAUAUUUGAUGUG | CACAUCAAAUAUUCUUCAG | Chimp, Rat | [2174-2192] 3′UTR |
| 272 | CUUUGAAACUCAGCGGAAU | AUUCCGCUGAGUUUCAAAG | Chimp | [2031-2049] 3′UTR |
| 273 | AGUAACCAGAACAACUCAU | AUGAGUUGUUCUGGUUACU | | [1917-1935] ORF |
| 274 | GGUAUGCAGAGAUAUCCAG | CUGGAUAUCUCUGCAUACC | Chimp | [1622-1640] ORF |
| 275 | GGAGGCAUUGGAGUAACUC | GAGUUACUCCAAUGCCUCC | Chimp | [1530-1548] ORF |
| 276 | CUGGAGGCAUUGGAGUAAC | GUUACUCCAAUGCCUCCAG | Chimp | [1528-1546] ORF |
| 277 | CAUUUGAGGAAUCACUGAA | UUCAGUGAUUCCUCAAAUG | Chimp | [1486-1504] ORF |
| 278 | GUGUAUCUGCAUUAGAAAA | UUUUCUAAUGCAGAUACAC | Chimp | [1273-1291] ORF |
| 279 | AGCAAGACCUGGUCAGUAU | AUACUGACCAGGUCUUGCU | Chimp | [1235-1253] ORF |
| 280 | CACAGACUUGGCUUUGGAU | AUCCAAAGCCAAGUCUGUG | Chimp | [1081-1099] ORF |
| 281 | ACCAGCUCUCAGAAUAUUU | AAAUAUUCUGAGAGCUGGU | Chimp | [933-951] ORF |
| 282 | AGAUGACCGUCACAUUACA | UGUAAUGUGACGGUCAUCU | | [2129-2147] 3′UTR |
| 283 | CAAGAUGACCGUCACAUUA | UAAUGUGACGGUCAUCUUG | | [2127-2145] 3′UTR |
| 284 | GACUUUGAAACUCAGCGGA | UCCGCUGAGUUUCAAAGUC | Chimp | [2029-2047] 3′UTR |
| 285 | CUGAAAACUUUUGCCAUGA | UCAUGGCAAAAGUUUUCAG | Chimp | [1973-1991] ORF + 3′UTR |
| 286 | GUUGGUGUUUUCUGUUGUG | CACAACAGAAAACACCAAC | Chimp | [1883-1881] ORF |
| 287 | ACAGAUGGGAUACAGAAGA | UCUUCUGUAUCCCAUCUGU | Chimp, Rat | [1740-1758] ORF |
| 288 | GUCAAACAGAUGGGAUACA | UGUAUCCCAUCUGUUUGAC | Chimp, Rat | [1735-1753] ORF |
| 289 | GGCUUCCACUCAGUCUUUG | CAAAGACUGAGUGGAAGCC | | [104-122] 5′UTR |
| 290 | CUAGUCAAGACUCCGAAAU | AUUUCGGAGUCUUGACUAG | Chimp | [1405-1423] ORF |
| 291 | AGCUCUCAGAAUAUUUCCU | AGGAAAUAUUCUGAGAGCU | Chimp | [936-954] ORF |
| 292 | AACCAGCUCUCAGAAUAUU | AAUAUUCUGAGAGCUGGUU | Chimp | [932-950] ORF |
| 293 | AUAACCUCUUCUUUGUCUU | AAGACAAAGAAGAGGUUAU | Chimp | [820-838] ORF |
| 294 | CACAGAAGGUUCCAAGCAG | CUGCUUGGAACCUUCUGUG | Chimp | [496-514] ORF |
| 295 | GAAAACUUUUGCCAUGAAG | CUUCAUGGCAAAAGUUUUC | Chimp | [1975-1993] ORF + 3′UTR |
| 296 | AGCAAAAUAUAACAGAGGA | UCCUCUGUUAUAUUUUGCU | | [1838-1856] ORF |
| 297 | UCAAACAGAUGGGAUACAG | CUGUAUCCCAUCUGUUUGA | Chimp, Rat | [1738-1754] ORF |
| 298 | UCAGUCAAACAGAUGGGAU | AUCCCAUCUGUUUGACUGA | Chimp, Rat, Ms | [1732-1750] ORF |
| 299 | CCCUCACAAUGUGUCCAAC | GUUGGACACAUUGUGAGGG | Chimp | [1303-1321] ORF |
| 300 | CCAUUUACCCUCACAAUGU | ACAUUGUGAGGGUAAAUGG | Chimp | [1296-1314] ORF |
| 301 | AGACUUUACAGGUAUAUCC | GGAUAUACCUGUAAAGUCU | Chimp | [1131-1149] ORF |
| 302 | CAGAGUAUUUCUCAGAACA | UGUUCUGAGAAAUACUCUG | Chimp | [958-976] ORF |
| 303 | AGAAAACUGUUAGAUGCAC | GUGCAUCUAACAGUUUUCU | Chimp | [2222-2240] 3′UTR |
| 304 | GCCUCCAUCUAGUCAAGAC | GUCUUGACUAGAUGGAGGC | Chimp | [1397-1415] ORF |
| 305 | AUAAGCCAGUCACCAUCAU | AUGAUGGUGACUGGCUUAU | Chimp | [1158-1174] ORF |
| 306 | GGAGCAAUAAGCCAGUCAC | GUGACUGGCUUAUUGCUCC | Chimp | [1150-1168] ORF |
| 307 | ACCUUUCCCUGAAGGAUUU | AAAUCCUUCAGGGAAAGGU | Chimp | [986-1004] ORF |
| 308 | ACCUCUUCUUUGUCUUCUA | UAGAAGACAAAGAAGAGGU | Chimp, Rat, Ms | [823-841] ORF |
| 309 | CCGAAAUUCUGCCCUUCAU | AUGAAGGGCAGAAUUUCGG | Chimp | [1417-1435] ORF |
| 310 | AAGCCAGUCACCAUCAUUU | AAAUGAUGGUGACUGGCUU | Chimp | [1158-1176] ORF |
| 311 | CGGAGCAAUAAGCCAGUCA | UGACUGGCUUAUUGCUCCG | Chimp | [1149-1167] ORF |
| 312 | GUAUGGAAGAGCCCAGAUU | AAUCUGGGCUCUUCCAUAC | Chimp | [1048-1066] ORF |
| 313 | CCCUGAAGGAUUUUCAAAA | UUUUGAAAAUCCUUCAGGG | Chimp | [992-1010] ORF |
| 314 | CCCUCAACUUCUCAGUGAA | UUCACUGAGAAGUUGAGGG | Chimp | [619-637] ORF |
| 315 | CCAUGUGCCGAACACUCUU | AAGAGUGUUCGGCACAUGG | | [460-478] ORF |
| 316 | GGACCCAAUUCACUAUCCA | UGGAUAGUGAAUUGGGUCC | Chimp | [1881-1899] ORF |
| 317 | GCAUUGGAGUAACUCCAUU | AAUGGAGUUACUCCAAUGC | Chimp | [1534-1552] ORF |
| 318 | CCUUCAUUCAAUCUAGAAA | UUUCUAGAUUGAAUGAAGG | Chimp, Rat | [1429-1447] ORF |
| 319 | GGCCAGAGUAUCACUACCU | AGGUAGUGAUACUCUGGCC | Chimp | [358-376] ORF |
| 320 | CUCACAAUGUGUCCAACUG | CAGUUGGACACAUUGUGAG | Chimp | [1305-1323] ORF |
| 321 | CCAGUCACCAUCAUUUCGG | CCGAAAUGAUGGUGACUGG | Chimp | [1161-1179] ORF |
| 322 | CCAAGCUAAUUUUCCACAG | CUGUGGAAAAUUAGCUUGG | Chimp | [1087-1085] ORF |
| 323 | ACCAGAGUAUUUCUCAGAA | UUCUGAGAAAUACUCUGGU | Chimp | [956-974] ORF |
| 324 | GCCUCUACAUAUGCAAUAA | UUAUUGCAUAUGUAGAGGC | Chimp | [768-786] ORF |
| 325 | UGGUGGUGCUAUUCCUCAU | AUGAGGAAUAGCACCACCA | Chimp | [742-760] ORF |
| 326 | GGAUAAAAGCAGAACAUUC | GAAUGUUCUGCUUUUAUCC | Chimp | [533-551] ORF |
| 327 | UGGAUAAAAGCAGAACAUU | AAUGUUCUGCUUUUAUCCA | Chimp | [532-550] ORF |
| 328 | UUGGAUAAAAGCAGAACAU | AUGUUCUGCUUUUAUCCAA | Chimp | [531-549] ORF |
| 329 | GCCUCAGCAUCUGUUCUUA | UAAGAACAGAUGCUGAGGC | Chimp | [414-432] ORF |
| 330 | GUAUGUUGCAUAACAAGUU | AACUUGUUAUGCAACAUAC | Chimp | [1669-1687] ORF |
| 331 | UCCUUUUGGAAGUCCAUUU | AAAUGGACUUCCAAAAGGA | Chimp | [1472-1490] ORF |
| 332 | CCUCCAUCUAGUCAAGACU | AGUCUUGACUAGAUGGAGG | Chimp | [1398-1416] ORF |
| 333 | GCCAGAGUAUCACUACCUC | GAGGUAGUGAUACUCUGGC | Chimp | [359-377] ORF |
| 334 | CCUCACAAUGUGUCCAACU | AGUUGGACACAUUGUGAGG | Chimp | [1304-1322] ORF |
| 335 | GCAUUAGAAAAUCAUCCAU | AUGGAUGAUUUUCUAAUGC | Chimp | [1281-1299] ORF |
| 336 | GAAAUCCGAAUGGUCAAAG | CUUUGACCAUUCGGAUUUC | Chimp | [1206-1224] ORF |
| 337 | CAUAAGUCAUCCCUCAGAU | AUCUGAGGGAUGACUUAUG | Chimp | [1181-1199] ORF |
| 338 | UCAUAAGUCAUCCCUCAGA | UCUGAGGGAUGACUUAUGA | Chimp | [1180-1198] ORF |
| 339 | GUCACCAUCAUUUCGGUCA | UGACCGAAAUGAUGGUGAC | Chimp | [1164-1182] ORF |
| 340 | CAAUAACCCAGUCACCAUC | GAUGGUGACUGGCUUAUUG | Chimp | [1154-1172] ORF |
| 341 | AGAGUAUUUCUCAGAACAU | AUGUUCUGAGAAAUACUCU | Chimp | [959-977] ORF |
| 342 | GCAUCAGUCUUAACCGAAC | GUUCGGUUAAGACUGAUGC | Chimp | [915-934] ORF |
| 343 | UGCUGAAGUAUCAAACUAA | UUAGUUUGAUACUUCAGCA | Chimp | [874-892] ORF |
| 344 | UGGUGCUAUUCCUCAUGAU | AUCAUGAGGAAUAGCACCA | Chimp | [745-763] ORF |
| 345 | GGUGGUGCUAUUCCUCAUG | CAUGAGGAAUAGCACCACC | Chimp | [743-761] ORF |
| 346 | CUAGAAAACUUCUCUUCAC | GUGAAGAGAAGUUUUCUAG | Chimp | [691-709] ORF |
| 347 | CUCAACUUCUCAGUGAAUU | AAUUCACUGAGAAGUUGAG | Chimp | [621-639] ORF |
| 348 | CCAUAUUACCUGUGGUGUU | AACACCACAGGUAAUAUGG | Chimp | [551-569] ORF |
| 349 | CCGUCACAUUACAUGUUUA | UAAACAUGUAAUGUGACGG | | [2135-2153] 3′UTR |
| 350 | GCAAGACCUGGUCAGUAUA | UAUACUGACCAGGUCUUGC | Chimp | [1236-1254] ORF |
| 351 | AGCUAAUUUUCCACAGACU | AGUCUGUGGAAAAUUAGCU | Chimp | [1070-1088] ORF |
| 352 | CAAGCUAAUUUUCCACAGA | UCUGUGGAAAAUUAGCUUG | Chimp | [1068-1086] ORF |
| 353 | GAAGAUUUGUAUGGAAGAG | CUCUUCCAUACAAAUCUUC | Chimp | [1040-1058] ORF |
| 354 | GGGCUGCUGAAGUAUCAAA | UUUGAUACUUCAGCAGCCC | Chimp | [870-888] ORF |
| 355 | CCUCUUCUUUGUCUUCUAC | GUAGAAGACAAAGAAGAGG | Chimp, Rat, Ms | [824-842] ORF |
| 356 | UCAACUUCUCAGUGAAUUA | UAAUUCACUGAGAAGUUGA | Chimp | [622-640] ORF |
| 357 | UUGUUGGAUAAAAGCAGAA | UUCUGCUUUUAUCCAACAA | Chimp | [526-546] ORF |
| 358 | ACCUCUGCCUGUUCAUCUG | CAGAUGAACAGGCAGAGGU | | [286-304] ORF |
| 359 | AGCUGAACUUGCUCAAUCU | AGAUUGAGCAAGUUCAGCU | Chimp | [2283-2301] 3′UTR |
| 360 | AGGAGCUGAACUUGCUCAA | UUGAGCAAGUUCAGCUCCU | Chimp | [2280-2298] 3′UTR |
| 361 | GGAAACCAAAGAGACCCUG | CAGGGUCUCUUUGGUUUCC | Chimp | [2158-2176] 3′UTR |
| 362 | CUGUUGUGGACCCAAUUCA | UGAAUUGGGUCCACAACAG | Chimp | [1874-1892] ORF |
| 363 | GACCUGACUAUGUCAACAU | AUGUUGACAUAGUCAGGUC | Chimp | [1702-1720] ORF |
| 364 | UCCAUUUGCAUCAAUACUC | GAGUAUUGAUGCAAAUGGA | Chimp | [1547-1565] ORF |
| 365 | CCUUUUGGAAGUCCAUUUG | CAAAUGGACUUCCAAAAGG | Chimp | [1473-1491] ORF |
| 366 | CGAAAUUCUGCCCUUCAUU | AAUGAAGGGCAGAAUUUCG | Chimp | [1418-1436] ORF |
| 367 | CCCAGUGUAUCUGCAUUAG | CUAAUGCAGAUACACUGGG | Chimp | [1269-1287] ORF |
| 368 | CAGAGUUUACCCAGCACAA | UUGUGCUGGGUAAACUCUG | Chimp | [1015-1033] ORF |
| 369 | GCAGAGUUUACCCAGCACA | UGUGCUGGGUAAACUCUGC | Chimp | [1014-1032] ORF |
| 370 | UCAAAACCGGCAGAGUUUA | UAAACUCUGCCGGUUUUGA | Chimp | [1005-1023] ORF |
| 371 | CCUGAAGGAUUUUCAAAAC | GUUUUGAAAAUCCUUCAGG | Chimp | [993-1011] ORF |
| 372 | CUCAGAACAUUUUCAUGAA | UUCAUGAAAAUGUUCUGAG | Chimp | [968-986] ORF |
| 373 | UCUUCUUUGUCUUCUACAU | AUGUAGAAGACAAAGAAGA | Chimp, Rat, Ms | [826-844] ORF |
| 374 | AGCCUCUACAUAUGCAAUA | UAUUGCAUAUGUAGAGGCU | Chimp | [767-785] ORF |
| 375 | CCUCAACUUCUCAGUGAAU | AUUCACUGAGAAGUUGAGG | Chimp | [620-638] ORF |
| 376 | GGAGAACCAGGAGAUUGUU | AACAAUCUCCUGGUUCUCC | Chimp | [514-532] ORF |
| 377 | AAACCAAAGAGACCCUGAA | UUCAGGGUCUCUUUGGUUU | Chimp | [2160-2178] 3′UTR |
| 378 | CGUCACAUUACAUGUUUAA | UUAAACAUGUAAUGUGACG | | [2136-2154] 3′UTR |
| 379 | GGAAAAACAGUUGGUGUUU | AAACACCAACUGUUUUUCC | | [1854-1872] ORF |
| 380 | UGCAGAUUUACUCUGUAUG | CAUACAGAGUAAAUCUGCA | Chimp | [1655-1673] ORF |
| 381 | UUGCAGAUUUACUCUGUAU | AUACAGAGUAAAUCUGCAA | Chimp | [1654-1672] ORF |
| 382 | GUCCCAGUGUAUCUGCAUU | AAUGCAGAUACACUGGGAC | Chimp | [1267-1285] ORF |
| 383 | CAAGACCUGGUCAGUAUAU | AUAUACUGACCAGGUCUUG | Chimp | [1237-1255] ORF |
| 384 | GUAUAACCAAGGGCCAGAG | CUCUGGCCCUUGGUUAUAC | Chimp | [347-365] ORF |
| 385 | CUGUGCCGAAAGACUUUAC | GUAAAGUCUUUCGGCACAG | Chimp | [1121-1139] ORF |
| 386 | UGGCUUUGGAUUUCUGGAC | GUCCAGAAAUCCAAAGCCA | Chimp | [1089-1107] ORF |
| 387 | GCCCAGAUUCCAAGCUAAU | AUUAGCUUGGAAUCUGGGC | Chimp | [1058-1076] ORF |
| 388 | AGCCCAGAUUCCAAGCUAA | UUAGCUUGGAAUCUGGGCU | Chimp | [1057-1075] ORF |
| 389 | AGCACAAAUUUGUGAAGAU | AUCUUCACAAAUUUGUGCU | Chimp | [1027-1045] ORF |
| 390 | CCCAGCACAAAUUUGUGAA | UUCACAAAUUUGUGCUGGG | Chimp | [1024-1042] ORF |
| 391 | CUCAGUGAAUUACAGUGAA | UUCACUGUAAUUCACUGAG | | [629-647] ORF |
| 392 | GCAGGAGAACCAGGAGAUU | AAUCUCCUGGUUCUCCUGC | Chimp | [511-529] ORF |
| 393 | GAACACUCUUGGCUUACCU | AGGUAAGCCAAGAGUGUUC | Chimp | [469-487] ORF |
| 394 | GGAGCUGAACUUGCUCAAU | AUUGAGCAAGUUCAGCUCC | Chimp | [2261-2299] 3′UTR |
| 395 | UGGAAACCAAAGAGACCCU | AGGGUCUCUUUGGUUUCCA | Chimp | [2157-2175] 3′UTR |
| 396 | CCUCAGCAUCUGUUCUUAA | UUAAGAACAGAUGCUGAGG | Chimp | [415-433] ORF |
| 397 | CCAGCUGUACCUCAGUCAA | UUGACUGAGGUACAGCUGG | Chimp, Rat, Ms | [1721-1739] ORF |
| 398 | GCAGAUUUACUCUGUAUGU | ACAUACAGAGUAAAUCUGC | Chimp | [1656-1674] ORF |
| 399 | UCCGUUGGUUUGCAGAUUU | AAAUCUGCAAACCAACGGA | Chimp | [1645-1663] ORF |
| 400 | UGAUGGUCCUUUUGGAAGU | ACUUCCAAAAGGACCAUCA | Chimp | [1466-1484] ORF |
| 401 | CUGAAACCAAAGCAACAUU | AAUGUUGCUUUGGUUUCAG | Chimp, Rat, Ms | [1321-1339] ORF |
| 402 | GUGUCCAACUGAAACCAAA | UUUGGUUUCAGUUGGACAC | Chimp | [1313-1331] ORF |
| 403 | CUUUGGAUUUCUGGACCUU | AAGGUCCAGAAAUCCAAAG | Chimp | [1092-1110] ORF |
| 404 | GUGAAGAUUUGUAUGGAAG | CUUCCAUACAAAUCUUCAC | Chimp | [1038-1058] ORF |
| 405 | GCACAAAUUUGUGAAGAUU | AAUCUUCACAAAUUUGUGC | Chimp | [1028-1046] ORF |
| 406 | UCAGAACAUUUUCAUGAAC | GUUCAUGAAAAUGUUCUGA | Chimp | [969-987] ORF |
| 407 | CUCUCAGAAUAUUUCCUUA | UAAGGAAAUAUUCUGAGAG | Chimp | [938-956] ORF |
| 408 | CUGCUUUUCUGGAAAACCU | AGGUUUUCCAGAAAAGCAG | Chimp, Rat | [321-339] ORF |
| 409 | GGCUGCUGAAGUAUCAAAC | GUUUGAUACUUCAGCAGCC | Chimp | [871-889] ORF |
| 410 | GUCCUGCUUUUCUGGAAAA | UUUUCCAGAAAAGCAGGAC | Chimp, Rat | [318-336] ORF |
| 411 | CCUCUACAUAUGCAAUAAG | CUUAUUGCAUAUGUAGAGG | Chimp | [769-787] ORF |
| 412 | AUCUGGUGAAUGCCCUCAA | UUGAGGGCAUUCACCAGAU | Chimp | [607-625] ORF |
| 413 | CGAGGAUCACAGAAGGUUC | GAACCUUCUGUGAUCCUCG | Chimp | [489-507] ORF |
| 414 | CGAACACUCUUGGCUUACC | GGUAAGCCAAGAGUGUUCG | Chimp | [468-486] ORF |
| 415 | GUUUUUGGUUGAACAAAUG | CAUUUGUUCAACCAAAAAC | Chimp | [2332-2350] 3′UTR |
| 416 | UGAACUUGCUCAAUCUAAG | CUUAGAUUGAGCAAGUUCA | Chimp | [2286-2304] 3′UTR |
| 417 | UCACUUUUCAGUUCUCAAA | UUUGAGAACUGAAAAGUGA | Chimp | [2198-2216] 3′UTR |
| 418 | ACCAAAGAGACCCUGAAGA | UCUUCAGGGUCUCUUUGGU | Chimp | [2162-2180] 3′UTR |
| 419 | ACCGUCACAUUACAUGUUU | AAACAUGUAAUGUGACGGU | | [2134-2152] 3′UTR |
| 420 | GCGGAAUCAAUCAGCUGUG | CACAGCUGAUUGAUUCCGC | Chimp | [2043-2061] 3′UTR |
| 421 | UGAAACUCAGCGGAAUCAA | UUGAUUCCGCUGAGUUUCA | Chimp | [2034-2052] 3′UTR |
| 422 | UUAAACACCUCUGCCUGUU | AACAGGCAGAGGUGUUUAA | | [280-298] ORF |
| 423 | CUCAGCAUCUGUUCUUAAC | GUUAAGAACAGAUGCUGAG | Chimp | [416-434] ORF |
| 424 | AGAGAACAGACCUGACUAU | AUAGUCAGGUCUGUUCUCU | Chimp | [1694-1712] ORF |
| 425 | CAGAUUUACUCUGUAUGUU | AACAUACAGAGUAAAUCUG | Chimp | [1657-1675] ORF |
| 426 | GUCCUUCCGUUGGUUUGCA | UGCAAACCAACGGAAGGAC | Chimp | [1640-1658] ORF |
| 427 | AGCUUAGAAGACUAUACUU | AAGUAUAGUCUUCUAAGCU | Chimp | [1597-1615] ORF |
| 428 | CCCUGUUGGAUGACUGGAA | UUCCAGUCAUCCAACAGGG | | [1570-1588] ORF |
| 429 | GUCCUUUUGGAAGUCCAUU | AAUGGACUUCCAAAAGGAC | Chimp | [1471-1489] ORF |
| 430 | ACUACCUCCACCAGAUGUU | AACAUCUGGUGGAGGUAGU | Chimp | [370-388] ORF |
| 431 | CAGAGUAUCACUACCUCCA | UGGAGGUAGUGAUACUCUG | Chimp | [361-379] ORF |
| 432 | GGACCUUUGUGCCUGUACU | AGUACAGGCACAAAGGUCC | Chimp | [1104-1122] ORF |
| 433 | CUGGACCUUUGUGCCUGUA | UACAGGCACAAAGGUCCAG | Chimp | [1102-1120] ORF |
| 434 | AAGCUAAUUUUCCACAGAC | GUCUGUGGAAAAUUAGCUU | Chimp | [1089-1087] ORF |
| 435 | UCCAAGCUAAUUUUCCACA | UGUGGAAAAUUAGCUUGGA | Chimp | [1088-1084] ORF |
| 436 | CCCAGAUUCCAAGCUAAUU | AAUUAGCUUGGAAUCUGGG | Chimp | [1059-1077] ORF |
| 437 | GGAAGAGCCCAGAUUCCAA | UUGGAAUCUGGGCUCUUCC | Chimp | [1052-1070] ORF |
| 438 | UGAAGAUUUGUAUGGAAGA | UCUUCCAUACAAAUCUUCA | Chimp | [1039-1057] ORF |
| 439 | AGAGUUUACCCAGCACAAA | UUUGUGCUGGGUAAACUCU | Chimp | [1016-1034] ORF |
| 440 | AUAUUUCCUUACCAGAGUA | UACUCUGGUAAGGAAAUAU | Chimp | [946-964] ORF |
| 441 | GCUGCUGACGUUGCAUGUU | AACAUGCAACGUCAGCAGC | Chimp | [845-863] ORF |
| 442 | CUGGUAUACUCAUAACCUC | GAGGUUAUGAGUAUACCAG | Chimp | [809-827] ORF |
| 443 | CUCUACAUAUGCAAUAAGA | UCUUAUUGCAUAUGUAGAG | Chimp | [770-788] ORF |
| 444 | UGCAGCAAGAUACCGAGAU | AUCUCGGUAUCUUGCUGCA | Chimp | [665-683] ORF |
| 445 | UCAGUGAAUUACAGUGAAG | CUUCACUGUAAUUCACUGA | | [630-648] ORF |
| 446 | CUGAACUUGCUCAAUCUAA | UUAGAUUGAGCAAGUUCAG | Chimp | [2285-2303] 3′UTR |
| 447 | GCUGAACUUGCUCAAUCUA | UAGAUUGAGCAAGUUCAGC | Chimp | [2284-2302] 3′UTR |
| 448 | UGUUUAAUCUGGAAACCAA | UUGGUUUCCAGAUUAAACA | Chimp | [2148-2166] 3′UTR |
| 449 | AACUGAGUAACCAGAACAA | UUGUUCUGGUUACUCAGUU | | [1912-1930] ORF |
| 450 | CUCUUCAUAAACUGAGUAA | UUACUCAGUUUAUGAAGAG | Chimp | [1903-1921] ORF |
| 451 | ACCUGACUAUGUCAACAUC | GAUGUUGACAUAGUCAGGU | Chimp | [1703-1721] ORF |
| 452 | UCCUUCCGUUGGUUUGCAG | CUGCAAACCAACGGAAGGA | Chimp | [1641-1659] ORF |
| 453 | GAUAUCCAGUCCUUCCGUU | AACGGAAGGACUGGAUAUC | Chimp | [1632-1650] ORF |
| 454 | UGACUGGAAACCAUACAAG | CUUGUAUGGUUUCCAGUCA | Chimp, Rat, Ms | [1580-1598] ORF |
| 455 | CUAGAAAUUAUCCCAAGCU | AGCUUGGGAUAAUUUCUAG | Chimp | [1441-1459] ORF |
| 456 | UCCCAGUGUAUCUGCAUUA | UAAUGCAGAUACACUGGGA | Chimp | [1268-1288] ORF |
| 457 | AAGACCUGGUCAGUAUAUU | AAUAUACUGACCAGGUCUU | Chimp | [1238-1256] ORF |
| 458 | GGUCAUAAGUCAUCCCUCA | UGAGGGAUGACUUAUGACC | Chimp | [1178-1196] ORF |
| 459 | UCGGUCAUAAGUCAUCCCU | AGGGAUGACUUAUGACCGA | Chimp | [1176-1194] ORF |
| 460 | CAGUCACCAUCAUUUCGGU | ACCGAAAUGAUGGUGACUG | Chimp | [1162-1180] ORF |
| 461 | UGCCUGUACUGUGCCGAAA | UUUCGGCACAGUACAGGCA | Chimp | [1113-1131] ORF |
| 462 | AGAGCCCAGAUUCCAAGCU | AGCUUGGAAUCUGGGCUCU | Chimp | [1055-1073] ORF |
| 463 | AGGAUUUUCAAAACCGGCA | UGCCGGUUUUGAAAAUCCU | Chimp | [998-1016] ORF |
| 464 | AGAAAACUUCUCUUCACAA | UUGUGAAGAGAAGUUUUCU | Chimp | [693-711] ORF |
| 465 | UGAAUGCCCUCAACUUCUC | GAGAAGUUGAGGGCAUUCA | Chimp | [613-631] ORF |
| 466 | UGGUGAAUGCCCUCAACUU | AAGUUGAGGGCAUUCACCA | Chimp, Ms | [610-628] ORF |
| 467 | UCAUCUGGCUCUCCAUGAA | UUCAUGGAGAGCCAGAUGA | Chimp | [298-316] ORF |
| 468 | AGCAGGAGAACCAGGAGAU | AUCUCCUGGUUCUCCUGCU | Chimp | [510-528] ORF |
| 469 | GAGCUGAACUUGCUCAAUC | GAUUGAGCAAGUUCAGCUC | Chimp | [2282-2300] 3′UTR |
| 470 | GGAUUCAAGAACUCCCUAG | CUAGGGAGUUCUUGAAUCC | Chimp | [2259-2277] 3′UTR |
| 471 | GUGGAUUCAAGAACUCCCU | AGGGAGUUCUUGAAUCCAC | Chimp | [2257-2275] 3′UTR |
| 472 | UGAUUCACUUUUCAGUUCU | AGAACUGAAAAGUGAAUCA | Chimp | [2194-2212] 3′UTR |
| 473 | GACCGUCACAUUACAUGUU | AACAUGUAAUGUGACGGUC | | [2133-2151] 3′UTR |
| 474 | UCAGCUGUGUUAUGCCAAA | UUUGGCAUAACACAGCUGA | Chimp | [2053-2071] 3′UTR |
| 475 | GUCUUUCAGCUGAAAACUU | AAGUUUUCAGCUGAAAGAC | Chimp | [1964-1982] ORF + 3′UTR |
| 476 | CAGCUGUACCUCAGUCAAA | UUUGACUGAGGUACAGCUG | Chimp, Rat, Ms | [1722-1740] ORF |
| 477 | AUGUCAACAUCCAGCUGUA | UACAGCUGGAUGUUGACAU | Chimp | [1711-1729] ORF |
| 478 | AGACCUGACUAUGUCAACA | UGUUGACAUAGUCAGGUCU | Chimp | [1701-1719] ORF |
| 479 | CCAGUCCUUCCGUUGGUUU | AAACCAACGGAAGGACUGG | Chimp | [1637-1655] ORF |
| 480 | UCAACACCCUGUUGGAUGA | UCAUCCAACAGGGUGUUGA | | [1564-1982] ORF |
| 481 | GGAGUAACUCCAUUUGCAU | AUGCAAAUGGAGUUACUCC | Chimp | [1539-1557] ORF |
| 482 | CAUUGGAGUAACUCCAUUU | AAAUGGAGUUACUCCAAUG | Chimp | [1535-1553] ORF |
| 483 | GGGUUAAACACCUCUGCCU | AGGCAGAGGUGUUUAACCC | Ms | [277-295] ORF |
| 484 | UGGAAGUCCAUUUGAGGAA | UUCCUCAAAUGGACUUCCA | Chimp | [1478-1496] ORF |
| 485 | AUCACUACCUCCACCAGAU | AUCUGGUGGAGGUAGUGAU | Chimp | [367-385] ORF |
| 486 | ACUGAAACCAAAGCAACAU | AUGUUGCUUUGGUUUCAGU | Chimp, Rat, Ms | [1320-1338] ORF |
| 487 | UCACAAUGUGUCCAACUGA | UCAGUUGGACACAUUGUGA | Chimp | [1306-1324] ORF |
| 488 | GUCAGUAUAUUACUCUACA | UGUAGAGUAAUAUACUGAC | Chimp | [1246-1264] ORF |
| 489 | UCCGAAUGGUCAAAGAAAA | UUUUCUUUGACCAUUCGGA | Chimp | [1210-1228] ORF |
| 490 | CGGUCAUAAGUCAUCCCUC | GAGGGAUGACUUAUGACCG | Chimp | [1177-1195] ORF |
| 491 | CAGCACAAAUUUGUGAAGA | UCUUCACAAAUUUGUGCUG | Chimp | [1026-1044] ORF |
| 492 | UGAAGGAUUUUCAAAACCG | CGGUUUUGAAAAUCCUUCA | Chimp | [995-1013] ORF |
| 493 | CUGAAGGAUUUUCAAAACC | GGUUUUGAAAAUCCUUCAG | Chimp | [994-1012] ORF |
| 494 | CCUUACCAGAGUAUUUCUC | GAGAAAUACUCUGGUAAGG | Chimp | [952-970] ORF |
| 495 | CUCAGAAUAUUUCCUUACC | GGUAAGGAAAUAUUCUGAG | Chimp | [940-958] ORF |
| 496 | GCUGCUGAAGUAUCAAACU | AGUUUGAUACUUCAGCAGC | Chimp | [872-890] ORF |
| 497 | GGUAUACUCAUAACCUCUU | AAGAGGUUAUGAGUAUACC | Chimp | [811-829] ORF |
| 498 | GCUCAAUCUAAGGCUGAUU | AAUCAGCCUUAGAUUGAGC | Chimp | [2293-2311] 3′UTR |
| 499 | GAUGUGAUGAUUCACUUUU | AAAAGUGAAUCAUCACAUC | Chimp | [2187-2205] 3′UTR |
| 500 | UGUGGACCCAAUUCACUAU | AUAGUGAAUUGGGUCCACA | Chimp | [1876-1896] ORF |
| 501 | UCUGUAUGUUGCAUAACAA | UUGUUAUGCAACAUACAGA | Chimp | [1666-1684] ORF |
| 502 | GGCAUUGGAGUAACUCCAU | AUGGAGUUACUCCAAUGCC | Chimp | [1533-1551] ORF |
| 503 | GGUCCUUUUGGAAGUCCAU | AUGGACUUCCAAAAGGACC | Chimp | [1470-1488] ORF |
| 504 | UGCAUUAGAAAAUCAUCCA | UGGAUGAUUUUCUAAUGCA | Chimp | [1280-1296] ORF |
| 505 | CCAGUGUAUCUGCAUUAGA | UCUAAUGCAGAUACACUGG | Chimp | [1270-1298] ORF |
| 506 | CUACAUUGUCCCAGUGUAU | AUACACUGGGACAAUGUAG | Chimp, Rat | [1260-1278] ORF |
| 507 | GGUCAGUAUAUUACUCUAC | GUAGAGUAAUAUACUGACC | Chimp | [1245-1263] ORF |
| 508 | CAGGUAUAUCCGGAGCAAU | AUUGCUCCGGAUAUACCUG | Chimp | [1139-1157] ORF |
| 509 | GCUUUGGAUUUCUGGACCU | AGGUCCAGAAAUCCAAAGC | Chimp | [1091-1109] ORF |
| 510 | CCAGCACAAAUUUGUGAAG | CUUCACAAAUUUGUGCUGG | Chimp | [1025-1043] ORF |
| 511 | GCUCUCAGAAUAUUUCCUU | AAGGAAAUAUUCUGAGAGC | Chimp | [937-955] ORF |
| 512 | UGCUGACGUUGCAUGUUUC | GAAACAUGCAACGUCAGCA | Chimp | [847-865] ORF |
| 513 | ACAGCCUCUACAUAUGCAA | UUGCAUAUGUAGAGGCUGU | Chimp | [765-783] ORF |
| 514 | UCCUAGAAAACUUCUCUUC | GAAGAGAAGUUUUCUAGGA | Chimp | [689-707] ORF |
| 515 | GAACUGAAUGCAGCAAGAU | AUCUUGCUGCAUUCAGUUC | Chimp, Ms | [657-675] ORF |
| 516 | CUAUCUGUAUUUUCUCAGG | CCUGAGAAAAUACAGAUAG | Chimp, Ms | [571-589] ORF |
| 517 | UGCUCAAUCUAAGGCUGAU | AUCAGCCUUAGAUUGAGCA | Chimp | [2292-2310] 3′UTR |
| 518 | UCCCUAGUGAGGAGCUGAA | UUCAGCUCCUCACUAGGGA | Chimp | [2271-2289] 3′UTR |
| 519 | UGAUGUGAUGAUUCACUUU | AAAGUGAAUCAUCACAUCA | Chimp | [2156-2204] 3′UTR |
| 520 | GGCAAGAUGACCGUCACAU | AUGUGACGGUCAUCUUGCC | | [2125-2143] 3′UTR |
| 521 | AGCUGUGUUAUGCCAAAGA | UCUUUGGCAUAACACAGCU | Chimp | [2055-2073] 3′UTR |
| 522 | UGCACUGAAUUCAAGACUG | CAGUCUUGAAUUCAGUGCA | Chimp | [1778-1796] ORF |
| 523 | AGCCUCAGCAUCUGUUCUU | AAGAACAGAUGCUGAGGCU | Chimp | [413-431] ORF |
| 524 | AGCUGUACCUCAGUCAAAC | GUUUGACUGAGGUACAGCU | Chimp, Rat, Ms | [1723-1741] ORF |
| 525 | ACAGACCUGACUAUGUCAA | UUGACAUAGUCAGGUCUGU | Chimp | [1699-1717] ORF |
| 526 | UCCAGUCCUUCCGUUGGUU | AACCAACGGAAGGACUGGA | Chimp | [1636-1654] ORF |
| 527 | CAAUCUAGAAAUUAUCCCA | UGGGAUAAUUUCUAGAUUG | Chimp | [1437-1455] ORF |
| 528 | UCCGAAAUUCUGCCCUUCA | UGAAGGGCAGAAUUUCGGA | Chimp | [1416-1434] ORF |
| 529 | CUCCGAAAUUCUGCCCUUC | GAAGGGCAGAAUUUCGGAG | Chimp | [1415-1433] ORF |
| 530 | AGAUUUACUACUGCCUCCA | UGGAGGCAGUAGUAAAUCU | Chimp, Rat | [1385-1403] ORF |
| 531 | CGAGAUUUACUACUGCCUC | GAGGCAGUAGUAAAUCUCG | Chimp, Rat | [1383-1401] ORF |
| 532 | UCUACAUUGUCCCAGUGUA | UACACUGGGACAAUGUAGA | Chimp, Rat | [1259-1277] ORF |
| 533 | UCAUGGAAAUCCGAAUGGU | ACCAUUCGGAUUUCCAUGA | Chimp | [1201-1219] ORF |
| 534 | UCCCUCAGAUGUCAUGGAA | UUCCAUGACAUCUGAGGGA | Chimp, Rat | [1190-1208] ORF |
| 535 | ACCAUCAUUUCGGUCAUAA | UUAUGACCGAAAUGAUGGU | Chimp | [1167-1185] ORF |
| 536 | UCCGGAGCAAUAAGCCAGU | ACUGGCUUAUUGCUCCGGA | Chimp | [1147-1165] ORF |
| 537 | ACAGGUAUAUCCGGAGCAA | UUGCUCCGGAUAUACCUGU | Chimp | [1138-1158] ORF |
| 538 | CCACUCAGUCUUUGACCCU | AGGGUCAAAGACUGAGUGG | | [109-127] 5′UTR |
| 539 | GAACCUUUCCCUGAAGGAU | AUCCUUCAGGGAAAGGUUC | Chimp | [984-1002] ORF |
| 540 | CAGAACAUUUUCAUGAACC | GGUUCAUGAAAAUGUUCUG | Chimp | [970-988] ORF |
| 541 | UCUCAGAAUAUUUCCUUAC | GUAAGGAAAUAUUCUGAGA | Chimp | [939-957] ORF |
| 542 | AUCCUAGAAAACUUCUCUU | AAGAGAAGUUUUCUAGGAU | Chimp | [688-706] ORF |
| 543 | GGCUCUCCAUGAAUGUCCU | AGGACAUUCAUGGAGACCC | Chimp | [304-322] ORF |
| 544 | UGCCGAACACUCUUGGCUU | AAGCCAAGAGUGUUCGGCA | Chimp | [465-483] ORF |
| 545 | CAAAUGCAAGAUUGAACAA | UUGUUCAAUCUUGCAUUUG | Chimp | [2345-2363] 3′UTR |
| 546 | UUGAUGUGAUGAUUCACUU | AAGUGAAUCAUCACAUCAA | Chimp | [2185-2203] 3′UTR |
| 547 | UCAGCGGAAUCAAUCAGCU | AGCUGAUUGAUUCCGCUGA | Chimp | [2040-2058] 3′UTR |
| 548 | UGAGUAACCAGAACAACUC | GAGUUGUUCUGGUUACUCA | | [1915-1933] ORF |
| 549 | UCCAGCUGUACCUCAGUCA | UGACUGAGGUACAGCUGGA | Chimp, Rat, | [1720-1738] ORF |
| | | | Ms | |
| 550 | GGCAAGAGAACAGACCUGA | UCAGGUCUGUUCUCUUGCC | Chimp | [1690-1708] ORF |
| 551 | UUCCGUUGGUUUGCAGAUU | AAUCUGCAAACCAACGGAA | Chimp | [1644-1662] ORF |
| 552 | GGAUGACUGGAAACCAUAC | GUAUGGUUUCCAGUCAUCC | Chimp, Rat, Ms | [1577-1595] ORF |
| 553 | ACACCCUGUUGGAUGACUG | CAGUCAUCCAACAGGGUGU | | [1567-1585] ORF |
| 554 | GUCCAUUUGAGGAAUCACU | AGUGAUUCCUCAAAUGGAC | Chimp | [1483-1501] ORF |
| 555 | UGGUCCUUUUGGAAGUCCA | UGGACUUCGAAAAGGACCA | Chimp | [1469-1487] ORF |
| 556 | AGACCUGGUCAGUAUAUUA | UAAUAUACUGACCAGGUCU | Chimp | [1239-1257] ORF |
| 557 | CCGAACCAGCUCUCAGAAU | AUUCUGAGAGCUGGUUCGG | Chimp | [929-947] ORF |
| 558 | UCAGUCUUAACCGAACCAG | CUGGUUCGGUUAAGACUGA | Chimp | [919-937] ORF |
| 559 | GGCUGCAUCAGUCUUAACC | GGUUAAGACUGAUGCAGCC | Chimp | [912-930] ORF |
| 560 | UCCUGCUUUUCUGGAAAAC | GUUUUCCAGAAAAGCAGGA | Chimp, Rat | [319-337] ORF |
| 561 | UGGUGGUGGUGCUAUUCCU | AGGAAUAGCACCACCACCA | Chimp | [739-757] ORF |
| 562 | CCUAGAAAACUUCUCUUCA | UGAAGAGAAGUUUUCUAGG | Chimp | [690-708] ORF |
| 563 | CCGAGAUGAGGAUCCUAGA | UCUAGGAUCCUCAUCUCGG | Chimp | [677-695] ORF |
| 564 | AUGUGAUGAUUCACUUUUC | GAAAAGUGAAUCAUCACAU | Chimp | [2188-2206] 3′UTR |
| 565 | AGCGGAAUCAAUCAGCUGU | ACAGCUGAUUGAUUCCGCU | Chimp | [2042-2060] 3′UTR |
| 566 | AGAGUCUUUCAGCUGAAAA | UUUUCAGCUGAAAGACUCU | Chimp | [1961-1979] ORF + 3′UTR |
| 567 | ACAGUUGGUGUUUUCUGUU | AACAGAAAACACCAACUGU | Chimp | [1860-1878] ORF |
| 568 | GUCCUCGGUGGAAACUUUU | AAAAGUUUCCACCGAGGAC | Chimp, Ms | [1807-1825] ORF |
| 569 | CAAGAGAACAGACCUGACU | AGUCAGGUCUGUUCUCUUG | Chimp | [1692-1710] ORF |
| 570 | GGUUUGCAGAUUUACUCUG | CAGAGUAAAUCUGCAAACC | Chimp, Ms | [1651-1669] ORF |
| 571 | UGGAUGACUGGAAACCAUA | UAUGGUUUCCAGUCAUCCA | Chimp, Rat, Ms | [1576-1594] ORF |
| 572 | UGGAGUAACUCCAUUUGCA | UGCAAAUGGAGUUACUCCA | Chimp | [1538-1556] ORF |
| 573 | GGAAUCACUGAACUAUGAG | CUCAUAGUUCAGUGAUUCC | Chimp | [1493-1511] ORF |
| 574 | AGGUAUAUCCGGAGCAAUA | UAUUGCUCCGGAUAUACCU | Chimp | [1140-1158] ORF |
| 575 | CCUUUCCCUGAAGGAUUUU | AAAAUCCUUCAGGGAAAGG | Chimp | [987-1005] ORF |
| 576 | UCAGAAUAUUUCCUUACCA | UGGUAAGGAAAUAUUCUGA | Chimp | [941-959] ORF |
| 577 | UGCUUUUCUGGAAAACCUU | AAGGUUUUCCAGAAAAGCA | Chimp, Rat | [322-340] ORF |
| 578 | UGCAUCAGUCUUAACCGAA | UUCGGUUAAGACUGAUGCA | Chimp | [915-933] ORF |
| 579 | CUGCAUCAGUCUUAACCGA | UCGGUUAAGACUGAUGCAG | Chimp | [914-932] ORF |
| 580 | ACAUGCUGCUGACGUUGCA | UGCAACGUCAGCAGCAUGU | Chimp | [841-859] ORF |
| 581 | ACCGAGAUGAGGAUCCUAG | CUAGGAUCCUCAUCUCGGU | Chimp | [676-694] ORF |
| 582 | CUGUUCAUCUGGCUCUCCA | UGGAGAGCCAGAUGAACAG | | [294-312] ORF |
| 583 | AAGGUUCCAAGCAGGAGAA | UUCUCCUGCUUGGAACCUU | Chimp | [501-519] ORF |
| 584 | CUCAAUCUAAGGCUGAUUG | CAAUCAGCCUUAGAUUGAG | Chimp | [2294-2312] 3′UTR |
| 585 | UGGAUUCAAGAACUCCCUA | UAGGGAGUUCUUGAAUCCA | Chimp | [2256-2276] 3′UTR |
| 586 | ACUGUUAGAUGCACACUGU | ACAGUGUGCAUCUAACAGU | Chimp | [2227-2245] 3′UTR |
| 587 | CAGCUGUGUUAUGCCAAAG | CUUUGGCAUAACACAGCUG | Chimp | [2054-2072] 3′UTR |
| 588 | GCCAUGAAGCAGGACUCUA | UAGAGUCCUGCUUCAUGGC | Chimp | [1965-2003] 3′UTR |
| 589 | UCUUUCAGCUGAAAACUUU | AAAGUUUUCAGCUGAAAGA | Chimp | [1965-1983] ORF + 3′UTR |
| 590 | GAGUCUUUCAGCUGAAAAC | GUUUUCAGCUGAAAGACUC | Chimp | [1962-1980] ORF + 3′UTR |
| 591 | AGACUGUUUAUAGGACGUC | GACGUCCUAUAAACAGUCU | | [1791-1809] ORF |
| 592 | CUGACUAUGUCAACAUCCA | UGGAUGUUGACAUAGUCAG | Chimp | [1705-1723] ORF |
| 593 | UGGCAAGAGAACAGACCUG | CAGGUCUGUUCUCUUGCCA | Chimp | [1689-1707] ORF |
| 594 | UCAUCCCUCAGAUGUCAUG | CAUGACAUCUGAGGGAUGA | Chimp, Rat | [1167-1205] ORF |
| 595 | GAACAUUUUCAUGAACCUU | AAGGUUCAUGAAAAUGUUC | Chimp | [972-990] ORF |
| 596 | CAGCCUCUACAUAUGCAAU | AUUGCAUAUGUAGAGGCUG | Chimp | [768-784] ORF |
| 597 | CACAGCCUCUACAUAUGCA | UGCAUAUGUAGAGGCUGUG | Chimp | [764-782] ORF |
| 598 | AGGAGAACCAGGAGAUUGU | ACAAUCUCCUGGUUCUCCU | Chimp | [513-531] ORF |
| 599 | GAAGGUUCCAAGCAGGAGA | UCUCCUGCUUGGAACCUUC | Chimp | [500-518] ORF |
| 600 | CUAAGACUUUGAAACUCAG | CUGAGUUUCAAAGUCUUAG | Chimp | [2025-2043] 3′UTR |
| 601 | AAGAGUCUUUCAGCUGAAA | UUUCAGCUGAAAGACUCUU | Chimp | [1960-1978] ORF + 3′UTR |
| 602 | GAAAAACAGUUGGUGUUUU | AAAACACCAACUGUUUUUC | | [1855-1873] ORF |
| 603 | GCAAGAGAACAGACCUGAC | GUCAGGUCUGUUCUCUUGC | Chimp | [1691-1709] ORF |
| 604 | UGAACUAUGAGGUCAGCCU | AGGCUGACCUCAUAGUUCA | Chimp | [1501-1519] ORF |
| 605 | CACUGAACUAUGAGGUCAG | CUGACCUCAUAGUUCAGUG | Chimp | [1498-1516] ORF |
| 606 | UCACUGAACUAUGAGGUCA | UGACCUCAUAGUUCAGUGA | Chimp | [1497-1515] ORF |
| 607 | UGGAAAACCUUCUUGCUGU | ACAGCAAGAAGGUUUUCCA | Chimp | [330-348] ORF |
| 608 | CAGCAAGAUACCGAGAUGA | UCAUCUCGGUAUCUUGCUG | Chimp | [667-685] ORF |
| 609 | UCAAUCUAAGGCUGAUUGU | ACAAUCAGCCUUAGAUUGA | Chimp | [2295-2313] 3′UTR |
| 610 | AGAACUCCCUAGUGAGGAG | CUCCUCACUAGGGAGUUCU | Chimp | [2266-2284] 3′UTR |
| 611 | CACUUUUCAGUUCUCAAAU | AUUUGAGAACUGAAAAGUG | Chimp | [2199-2217] 3′UTR |
| 612 | UGAUGAUUCACUUUUCAGU | ACUGAAAAGUGAAUCAUCA | Chimp | [2191-2209] 3′UTR |
| 613 | AGCUGAAAACUUUUGCCAU | AUGGCAAAAGUUUUCAGCU | Chimp | [1971-1989] ORF + 3′UTR |
| 614 | CAGCUGAAAACUUUUGCCA | UGGCAAAAGUUUUCAGCUG | Chimp | [1970-1988] ORF + 3′UTR |
| 615 | AGACUCUUCAUAAACUGAG | CUCAGUUUAUGAAGAGUCU | Chimp | [1900-1918] ORF |
| 616 | CUGUACCUCAGUCAAACAG | CUGUUUGACUGAGGUACAG | Chimp, Rat, Ms | [1725-1743] ORF |
| 617 | ACAUCCAGCUGUACCUCAG | CUGAGGUACAGCUGGAUGU | Chimp, Rat, Ms | [1717-1735] ORF |
| 618 | UCAACAUCCAGCUGUACCU | AGGUACAGCUGGAUGUUGA | Chimp | [1714-1732] ORF |
| 619 | AUGACUGGAAACCAUACAA | UUGUAUGGUUUCCAGUCAU | Chimp, Rat, Ms | [1579-1597] ORF |
| 620 | AGGCAUUGGAGUAACUCCA | UGGAGUUACUCCAAUGCCU | Chimp | [1532-1550] ORF |
| 621 | ACCCAGCACAAAUUUGUGA | UCACAAAUUUGUGCUGGGU | Chimp | [1023-1041] ORF |
| 622 | ACCGAACCAGCUCUCAGAA | UUCUGAGAGCUGGUUCGGU | Chimp | [928-948] ORF |
| 623 | UGUCCUGCUUUUCUGGAAA | UUUCCAGAAAAGCAGGACA | Chimp, Rat | [317-335] ORF |
| 624 | CUGUUAGAUGCACACUGUU | AACAGUGUGCAUCUAACAG | Chimp, Rat | [2228-2246] 3′UTR |
| 625 | GAAAACUGUUAGAUGCACA | UGUGCAUCUAACAGUUUUC | Chimp | [2223-2241] 3′UTR |
| 626 | UGUGAUGAUUCACUUUUCA | UGAAAAGUGAAUCAUCACA | Chimp | [2189-2207] 3′UTR |
| 627 | ACUCUUCAUAAACUGAGUA | UACUCAGUUUAUGAAGAGU | Chimp | [1902-1920] ORF |
| 628 | GACUCUUCAUAAACUGAGU | ACUCAGUUUAUGAAGAGUC | Chimp | [1901-1919] ORF |
| 629 | CAGAGCCUCAGCAUCUGUU | AACAGAUGCUGAGGCUCUG | Chimp | [410-428] ORF |
| 630 | CUAAGCAGAGCCUCAGCAU | AUGCUGAGGCUCUGCUUAG | Chimp | [405-423] ORF |
| 631 | UUGGAUGACUGGAAACCAU | AUGGUUUCCAGUCAUCCAA | Chimp | [1575-1593] ORF |
| 632 | ACAUUUUCAUGAACCUUUC | GAAAGGUUCAUGAAAAUGU | Chimp | [974-992] ORF |
| 633 | AGAACAUUUUCAUGAACCU | AGGUUCAUGAAAAUGUUCU | Chimp | [971-989] ORF |
| 634 | GAUUCACUUUUCAGUUCUC | GAGAACUGAAAAGUGAAUC | Chimp | [2195-2213] 3′UTR |
| 635 | UGGAAAUCCGAAUGGUCAA | UUGACCAUUCGGAUUUCCA | Chimp | [1204-1222] ORF |
| 636 | UCCCUGAAGGAUUUUCAAA | UUUGAAAAUCCUUCAGGGA | Chimp | [991-1009] ORF |
| 637 | GGAUCACAGAAGGUUCCAA | UUGGAACCUUCUGUGAUCC | Chimp | [492-510] ORF |
| 638 | CAGAAUAUUUCCUUACCAG | CUGGUAAGGAAAUAUUCUG | Chimp | [942-960] ORF |
| 639 | AAAAACAGUUGGUGUUUUC | GAAAACACCAACUGUUUUU | | [1856-1874] ORF |
| 640 | GGCUUUGGAUUUCUGGACC | GGUCCAGAAAUCCAAAGCC | Chimp | [1090-1108] ORF |
| 641 | GUCUUCUACAUGCUGCUGA | UCAGCAGCAUGUAGAAGAC | Chimp | [834-852] ORF |
| 642 | GUUGAACUGAAUGCAGCAA | UUGCUGCAUUCAGUUCAAC | Chimp | [654-672] ORF |
| 643 | AUGUCCUGCUUUUCUGGAA | UUCCAGAAAAGCAGGACAU | Chimp, Rat | [316-334] ORF |
| 644 | UGCCUGUUCAUCUGGCUCU | AGAGCCAGAUGAACAGGCA | | [291-309] ORF |

TABLE B
21-mer oligomers for NOX4 (NADPH oxidase 4)

| | | | | |
|---|---|---|---|---|
| | | | | Human-20149638 |
| Number | Sense siRNA | AntiSense siRNA | Other Sp | ORF: 240-1976 |

| | | | | |
|---|---|---|---|---|
| 1 | UCAUCCAUUUACCCUCACAAU | AUUGUGAGGGUAAAUGGAUGA | Chimp | [1292-1312] ORF |
| 2 | GGGACAAGAUUUGAAUACAAU | AUUGUAUUCAAAUCUUGUCCC | | [1938-1958] ORF |
| 3 | GCCUUAUCCUUUUACCCAUGU | ACAUGGGUAAAAGGAUAAGGC | | [445-465] ORF |
| 4 | GCCAAAGAAUAGUAAGGUUUU | AAAACCUUACUAUUCUUUGGC | Chimp | [2068-2086] 3′UTR |
| 5 | GGAAUCAAUCAGCUGUGUUAU | AUAACACAGCUGAUUGAUUCC | Chimp | [2045-2065] 3′UTR |
| 6 | CGGUGGAAACUUUUGUUUGAU | AUCAAACAAAAGUUUCCACCG | Chimp | [1812-1832] ORF |
| 7 | UGCCCUUCAUUCAAUCUAGAA | UUCUAGAUUGAAUGAAGGGCA | Chimp, Rat | [1426-1446] ORF |
| 8 | GGACAGAACGAUUUCGAGAUU | AAUCUCGAAAUCGUUCUGUCC | Chimp | [1369-1389] ORF |
| 9 | AUCAUCCAUUUACCCUCACAA | UUGUGAGGGUAAAUGGAUGAU | Chimp | [1291-1311] ORF |
| 10 | GCCGAAAGACUUUACAGGUAU | AUACCUGUAAAGUCUUUCGGC | Chimp | [1125-1145] ORF |
| 11 | GGAUUUUCAAAACCGGCAGAG | CUCUGCCGGUUUUGAAAAUCC | Chimp | [999-1019] ORF |
| 12 | GCAGGACUCUAAAGAAGGAAU | AUUCCUUCUUUAGAGUCCUGC | Chimp | [1993-2013] 3′UTR |
| 13 | CCCAAUUCACUAUCCAAGACU | AGUCUUGGAUAGUGAAUUGGG | Chimp | [1884-1904] ORF |
| 14 | GCAUCUGUUCUUAACCUCAAC | GUUGAGGUUAAGAACAGAUGC | Chimp | [420-440] ORF |
| 15 | ACAGAACGAUUUCGAGAUUUA | UAAAUCUCGAAAUCGUUCUGU | Chimp | [1371-1391] ORF |
| 16 | CCGAAAGACUUUACAGGUAUA | UAUACCUGUAAAGUCUUUCGG | Chimp | [1126-1148] ORF |
| 17 | CGAGAUGAGGAUCCUAGAAAA | UUUUCUAGGAUCCUCAUCUCG | Chimp | [678-698] ORF |
| 18 | ACAACUCAUAUGGGACAAGAU | AUCUUGUCCCAUAUGAGUUGU | | [1927-1947] ORF |
| 19 | GCAUAACAAGUUUUGGCAAGA | UCUUGCCAAAACUUGUUAUGC | Chimp, Rat, Ms | [1676-1696] ORF |
| 20 | GUAACUCCAUUUGCAUCAAUA | UAUUGAUGCAAAUGGAGUUAC | Chimp | [1542-1562] ORF |
| 21 | CCAAGGGCCAGAGUAUCACUA | UAGUGAUACUCUGGCCCUUGG | Chimp | [353-373] ORF |
| 22 | AUCAUUUCGGUCAUAAGUCAU | AUGACUUAUGACCGAAAUGAU | Chimp | [1170-1190] ORF |
| 23 | CGAAAGACUUUACAGGUAUAU | AUAUACCUGUAAAGUCUUUCG | Chimp | [1127-1147] ORF |
| 24 | UCAUGAUCACAGCCUCUACAU | AUGUAGAGGCUGUGAUCAUGA | Chimp | [757-777] ORF |
| 25 | GUGGUGUUACUAUCUGUAUUU | AAAUACAGAUAGUAACACCAC | Chimp | [562-582] ORF |
| 26 | CCUUAUCCUUUUACCCAUGUG | CACAUGGGUAAAAGGAUAAGG | | [446-466] ORF |
| 27 | UGCCAAAGAAUAGUAAGGUUU | AAACCUUACUAUUCUUUGGCA | Chimp | [2065-2085] 3′UTR |
| 28 | GUGUUAUGCCAAAGAAUAGUA | UACUAUUCUUUGGCAUAACAC | Chimp | [2059-2079] 3′UTR |
| 29 | UGGGACAAGAUUUGAAUACAA | UUGUAUUCAAAUCUUGUCCCA | | [1937-1957] ORF |
| 30 | CCAAUUCACUAUCCAAGACUC | GAGUCUUGGAUAGUGAAUUGG | Chimp | [1885-1905] ORF |
| 31 | GGAGAAAAAUAUCAUGCACUG | CAGUGCAUGAUAUUUUUCUCC | Chimp | [1764-1784] ORF |
| 32 | AUCAAUACUCAACACCCUGUU | AACAGGGUGUUGAGUAUUGAU | | [1556-1576] ORF |
| 33 | GAGUUUACCCAGCACAAAUUU | AAAUUUGUGCUGGGUAAACUC | Chimp | [1017-1037] ORF |
| 34 | UCACAACUGUUCCUGGCCUGA | UCAGGCCAGGAACAGUUGUGA | Chimp | [706-726] ORF |
| 35 | GGUGUUACUAUCUGUAUUUUC | GAAAAUACAGAUAGUAACACC | Chimp | [564-584] ORF |
| 36 | CCAGGAGAUUGUUGGAUAAAA | UUUUAUCCAACAAUCUCCUGG | Chimp | [520-540] ORF |
| 37 | UGGUUGAACAAAUGCAAGAUU | AAUCUUGCAUUUGUUCAACCA | Chimp | [2337-2357] 3′UTR |
| 38 | AAAGAAGGAAUGAGUGCAAUU | AAUUGCACUCAUUCCUUCUUU | Chimp | [2003-2023] 3′UTR |
| 39 | CUAAAGAAGGAAUGAGUGCAA | UUGCACUCAUUCCUUCUUUAG | Chimp | [2001-2021] 3′UTR |
| 40 | GACCCAAUUCACUAUCCAAGA | UCUUGGAUAGUGAAUUGGGUC | Chimp | [1862-1902] ORF |
| 41 | CUGUAUAUUGAUGGUCCUUUU | AAAAGGACCAUCAAUAUACAG | Chimp | [1458-1478] ORF |
| 42 | CUGCCCUUCAUUCAAUCUAGA | UCUAGAUUGAAUGAAGGGCAG | Chimp, Rat | [1425-1445] ORF |
| 43 | CUACUGCCUCCAUCUAGUCAA | UUGACUAGAUGGAGGCAGUAG | Chimp | [1392-1412] ORF |
| 44 | GGAGACUGGACAGAACGAUUU | AAAUCGUUCUGUCCAGUCUCC | Chimp | [1362-1382] ORF |
| 45 | GGGUUCAUCUUAAAAUAGUAG | CUACUAUUUUAAGAUGAACCC | Chimp | [1342-1362] ORF |
| 46 | CCGAAUGGUCAAAGAAAAUUU | AAAUUUUCUUUGACCAUUCGG | Chimp | [1211-1231] ORF |
| 47 | CCAGAUUCCAAGCUAAUUUUC | GAAAAUUAGCUUGGAAUCUGG | Chimp | [1060-1080] ORF |
| 48 | GAAAACCUUCUUGCUGUAUAA | UUAUACAGCAAGAAGGUUUUC | Chimp | [332-352] ORF |
| 49 | AAACUUCUCUUCACAACUGUU | AACAGUUGUGAAGAGAAGUUU | Chimp | [696-716] ORF |
| 50 | AGCCUUAUCCUUUUACCCAUG | CAUGGGUAAAAGGAUAAGGCU | | [444-464] ORF |
| 51 | CGGAAUCAAUCAGCUGUGUUA | UAACACAGCUGAUUGAUUCCG | Chimp | [2044-2064] 3′UTR |
| 52 | UGAGUGCAAUUUCUAAGACUU | AAGUCUUAGAAAUUGCACUCA | Chimp | [2013-2033] 3′UTR |
| 53 | GGACUCUAAAGAAGGAAUGAG | CUCAUUCCUUCUUUAGAGUCC | Chimp | [1996-2016] 3′UTR |
| 54 | CCAGAACAACUCAUAUGGGAC | GUCCCAUAUGAGUUGUUCUGG | | [1922-1942] ORF |
| 55 | GUGGAAACUUUUGUUUGAUGA | UCAUCAAACAAAAGUUUCCAC | Chimp | [1814-1834] ORF |
| 56 | CGAUUUCGAGAUUUACUACUG | CAGUAGUAAAUCUCGAAAUCG | Chimp | [1377-1397] ORF |
| 57 | CAAACUAAUUUAGAUACCCAC | GUGGGUAUCUAAAUUAGUUUG | Chimp | [885-905] ORF |
| 58 | CCUCAUGAUCACAGCCUCUAC | GUAGAGGCUGUGAUCAUGAGG | Chimp | [755-775] ORF |
| 59 | GAAGACUUUGUUGAACUGAAU | AUUCAGUUCAACAAAGUCUUC | | [645-685] ORF |
| 60 | GUAUUUUCUCAGGCGUGCAUG | CAUGCACGCCUGAGAAAAUAC | Chimp | [577-597] ORF |
| 61 | CUGUAUUUUCUCAGGCGUGCA | UGCACGCCUGAGAAAAUACAG | Chimp | [575-595] ORF |
| 62 | CGUGUUCCUCUUUAAAUUGUU | AACAAUUUAAAGAGGAACACG | Chimp | [2314-2334] 3′UTR |
| 63 | CAAAGAGACCCUGAAGAAUAU | AUAUUCUUCAGGGUCUCUUUG | Chimp | [2164-2184] 3′UTR |
| 64 | AGAAGGAAUGAGUGCAAUUUC | GAAAUUGCACUCAUUCCUUCU | Chimp | [2005-2025] 3′UTR |
| 65 | AGGACUCUAAAGAAGGAAUGA | UCAUUCCUUCUUUAGAGUCCU | Chimp | [1995-2015] 3′UTR |
| 66 | AGAACAACUCAUAUGGGACAA | UUGUCCCAUAUGAGUUGUUCU | | [1924-1944] ORF |
| 67 | GGUGGAAACUUUUGUUUGAUG | CAUCAAACAAAAGUUUCCACC | Chimp | [1813-1833] ORF |
| 68 | CACUGAAUUCAAGACUGUUUA | UAAACAGUCUUGAAUUCAGUG | Chimp | [1780-1800] ORF |
| 69 | UAACAAGUUUUGGCAAGAGAA | UUCUCUUGCCAAAACUUGUUA | Chimp | [1679-1699] ORF |
| 70 | GAGUAACUCCAUUUGCAUCAA | UUGAUGCAAAUGGAGUUACUC | Chimp | [1540-1560] ORF |
| 71 | AGCUGUAUAUUGAUGGUCCUU | AAGGACCAUCAAUAUACAGCU | Chimp | [1456-1476] ORF |
| 72 | GACAGAACGAUUUCGAGAUUU | AAAUCUCGAAAUCGUUCUGUC | Chimp | [1370-1390] ORF |
| 73 | CUAUGAUAUCUUCUGGUAUAC | GUAUACCAGAAGAUAUCAUAG | Chimp | [797-817] ORF |
| 74 | GUGAAUUACAGUGAAGACUUU | AAAGUCUUCACUGUAAUUCAC | | [633-653] ORF |
| 75 | AGUGAAUUACAGUGAAGACUU | AAGUCUUCACUGUAAUUCACU | | [632-652] ORF |
| 76 | CCUGUGGUGUUACUAUCUGUA | UACAGAUAGUAACACCACAGG | Chimp | [559-579] ORF |
| 77 | CAGGAGAUUGUUGGAUAAAAG | CUUUUAUCCAACAAUCUCCUG | Chimp | [521-541] ORF |
| 78 | GGUUGAACAAAUGCAAGAUUG | CAAUCUUGCAUUUGUUCAACC | Chimp | [2338-2358] 3′UTR |
| 79 | CCUUUUACCCAUGUGCCGAAC | GUUCGGCACAUGGGUAAAAGG | | [452-472] ORF |
| 80 | AAAGAGACCCUGAAGAAUAUU | AAUAUUCUUCAGGGUCUCUUU | Chimp | [2165-2185] 3′UTR |
| 81 | ACAUGUUUAAUCUGGAAACCA | UGGUUUCCAGAUUAAACAUGU | Chimp | [2145-2165] 3′UTR |
| 82 | CCAAAGAAUAGUAAGGUUUUC | GAAAACCUUACUAUUCUUUGG | Chimp | [2067-2087] 3′UTR |
| 83 | AUGCCAAAGAAUAGUAAGGUU | AACCUUACUAUUCUUUGGCAU | Chimp | [2064-2084] 3′UTR |
| 84 | GAGUGCAAUUUCUAAGACUUU | AAAGUCUUAGAAAUUGCACUC | Chimp | [2014-2034] 3′UTR |
| 85 | CAGGACUCUAAAGAAGGAAUG | CAUUCCUUCUUUAGAGUCCUG | Chimp | [1994-2014] 3′UTR |
| 86 | AGCAGGACUCUAAAGAAGGAA | UUCCUUCUUUAGAGUCCUGCU | Chimp | [1992-2012] 3′UTR |
| 87 | CAAUAAAGAGUCUUUCAGCUG | CAGCUGAAAGACUCUUUAUUG | Chimp | [1955-1975] ORF |
| 88 | CAACUCAUAUGGGACAAGAUU | AAUCUUGUCCCAUAUGAGUUG | | [1928-1948] ORF |
| 89 | UCACUAUCCAAGACUCUUCAU | AUGAAGAGUCUUGGAUAGUGA | Chimp | [1890-1910] ORF |
| 90 | CAAUUCACUAUCCAAGACUCU | AGAGUCUUGGAUAGUGAAUUG | Chimp | [1886-1906] ORF |
| 91 | CCUCGGUGGAAACUUUUGUUU | AAACAAAAGUUUCCACCGAGG | Chimp | [1809-1829] ORF |
| 92 | AGAUUUACUCUGUAUGUUGCA | UGCAACAUACAGAGUAAAUCU | Chimp | [1658-1678] ORF |
| 93 | AGUAACUCCAUUUGCAUCAAU | AUUGAUGCAAAUGGAGUUACU | Chimp | [1541-1561] ORF |
| 94 | CCAUUUGAGGAAUCACUGAAC | GUUCAGUGAUUCCUCAAAUGG | Chimp | [1485-1505] ORF |
| 95 | GAACGAUUUCGAGAUUUACUA | UAGUAAAUCUCGAAAUCGUUC | Chimp | [1374-1394] ORF |
| 96 | AGGAGACUGGACAGAACGAUU | AAUCGUUCUGUCCAGUCUCCU | Chimp, Ms | [1361-1381] ORF |
| 97 | GUCCAACUGAAACCAAAGCAA | UUGCUUUGGUUUCAGUUGGAC | Chimp | [1315-1335] ORF |
| 98 | CCUGGUCAGUAUAUUACUCUA | UAGAGUAAUAUACUGACCAGG | Chimp | [1242-1262] ORF |
| 99 | ACCUUCUUGCUGUAUAACCAA | UUGGUUAUACAGCAAGAAGGU | Chimp | [336-356] ORF |
| 100 | GGAAAACCUUCUUGCUGUAUA | UAUACAGCAAGAAGGUUUUCC | Chimp | [331-351] ORF |
| 101 | AAAACUUCUCUUCACAACUGU | ACAGUUGUGAAGAGAAGUUUU | Chimp | [695-715] ORF |
| 102 | UGAAUUACAGUGAAGACUUUG | CAAAGUCUUCACUGUAAUUCA | | [634-654] ORF |
| 103 | GAAUGCCCUCAACUUCUCAGU | ACUGAGAAGUUGAGGGCAUUC | Chimp | [614-634] ORF |
| 104 | CUGUGGUGUUACUAUCUGUAU | AUACAGAUAGUAACACCACAG | Chimp | [560-580] ORF |
| 105 | UUGGUUGAACAAAUGCAAGAU | AUCUUGCAUUUGUUCAACCAA | Chimp | [2336-2356] 3′UTR |
| 106 | CUGUUGAUUUUCAUGGUGGAU | AUCCACCAUGAAAAUCAACAG | | [2242-2262] 3′UTR |
| 107 | CAGCCUUAUCCUUUUACCCAU | AUGGGUAAAAGGAUAAGGCUG | | [443-463] ORF |
| 108 | GCUGUGUUAUGCCAAAGAAUA | UAUUCUUUGGCAUAACACAGC | Chimp | [2056-2076] 3′UTR |
| 109 | AAGGAAUGAGUGCAAUUUCUA | UAGAAAUUGCACUCAUUCCUU | Chimp | [2007-2027] 3′UTR |
| 110 | UAAAGAAGGAAUGAGUGCAAU | AUUGCACUCAUUCCUUCUUUA | Chimp | [2002-2022] 3′UTR |
| 111 | GCUGAAAACUUUUGCCAUGAA | UUCAUGGCAAAAGUUUUCAGC | Chimp | [1972-1992] |
| | | | | ORF + 3′UTR |
| 112 | GAACAACUCAUAUGGGACAAG | CUUGUCCCAUAUGAGUUGUUC | | [1925-1945] ORF |
| 113 | AUAACAAGUUUUGGCAAGAGA | UCUCUUGCCAAAACUUGUUAU | Chimp | [1678-1698] ORF |
| 114 | UGCAGAGAUAUCCAGUCCUUC | GAAGGACUGGAUAUCUCUGCA | Chimp | [1626-1646] ORF |
| 115 | AUGCAGAGAUAUCCAGUCCUU | AAGGACUGGAUAUCUCUGCAU | Chimp | [1625-1645] ORF |
| 116 | UAACUCCAUUUGCAUCAAUAC | GUAUUGAUGCAAAUGGAGUUA | Chimp | [1543-1563] ORF |
| 117 | GGAUUGUGUCUAAGCAGAGCC | GGCUCUGCUUAGACACAAUCC | Chimp | [396-416] ORF |
| 118 | CAGAACGAUUUCGAGAUUUAC | GUAAAUCUCGAAAUCGUUCUG | Chimp | [1372-1392] ORF |
| 119 | GACUGGACAGAACGAUUUCGA | UCGAAAUCGUUCUGUCCAGUC | Chimp | [1365-1385] ORF |
| 120 | GAGACUGGACAGAACGAUUUC | GAAAUCGUUCUGUCCAGUCUC | Chimp | [1363-1383] ORF |
| 121 | ACCCUCACAAUGUGUCCAACU | AGUUGGACACAUUGUGAGGGU | Chimp | [1302-1322] ORF |
| 122 | GAACCAGCUCUCAGAAUAUUU | AAAUAUUCUGAGAGCUGGUUC | Chimp | [931-951] ORF |
| 123 | GUAUACUCAUAACCUCUUCUU | AAGAAGAGGUUAUGAGUAUAC | Chimp | [812-832] ORF |
| 124 | UGAUAUCUUCUGGUAUACUCA | UGAGUAUACCAGAAGAUAUCA | Chimp, Rat, Ms | [800-820] ORF |
| 125 | CCAUGAAUGUCCUGCUUUUCU | AGAAAAGCAGGACAUUCAUGG | Chimp | [310-330] ORF |
| 126 | UCCAUGAAUGUCCUGCUUUUC | GAAAAGCAGGACAUUCAUGGA | Chimp | [309-329] ORF |
| 127 | GCAAGAUACCGAGAUGAGGAU | AUCCUCAUCUCGGUAUCUUGC | Chimp | [669-689] ORF |
| 128 | GGAGAUUGUUGGAUAAAAGCA | UGCUUUUAUCCAACAAUCUCC | Chimp | [523-543] ORF |
| 129 | UCCUUUUACCCAUGUGCCGAA | UUCGGCACAUGGGUAAAAGGA | | [451-471] ORF |
| 130 | AGAGACCCUGAAGAAUAUUUG | CAAAUAUUCUUCAGGGUCUCU | Chimp | [2167-2187] 3′UTR |
| 131 | GCAGCCUUAUCCUUUUACCCA | UGGGUAAAAGGAUAAGGCUGC | | [442-462] ORF |
| 132 | AACUGCAGCCUUAUCCUUUUA | UAAAAGGAUAAGGCUGCAGUU | Chimp | [438-458] ORF |
| 133 | CAACUGCAGCCUUAUCCUUUU | AAAAGGAUAAGGCUGCAGUUG | Chimp | [437-457] ORF |
| 134 | AGACUUUGAAACUCAGCGGAA | UUCCGCUGAGUUUCAAAGUCU | Chimp | [2028-2048] 3′UTR |
| 135 | GUGCAAUUUCUAAGACUUUGA | UCAAAGUCUUAGAAAUUGCAC | Chimp | [2016-2036] 3′UTR |
| 136 | GAAUGAGUGCAAUUUCUAAGA | UCUUAGAAAUUGCACUCAUUC | Chimp | [2010-2030] 3′UTR |
| 137 | AUAAAGAGUCUUUCAGCUGAA | UUCAGCUGAAAGACUCUUUAU | Chimp | [1957-1977] |
| | | | | ORF + 3′UTR |
| 138 | AGAGGAAAAACAGUUGGUGUU | AACACCAACUGUUUUUCCUCU | | [1851-1871] ORF |
| 139 | AGCAUCUGUUCUUAACCUCAA | UUGAGGUUAAGAACAGAUGCU | Chimp | [419-439] ORF |
| 140 | UGGAGAAAAAUAUCAUGCACU | AGUGCAUGAUAUUUUUCUCCA | Chimp | [1763-1783] ORF |
| 141 | GGGUAUGCAGAGAUAUCCAGU | ACUGGAUAUCUCUGCAUACCC | Chimp | [1621-1641] ORF |
| 142 | GCUGUAUAUUGAUGGUCCUUU | AAAGGACCAUCAAUAUACAGC | Chimp | [1457-1477] ORF |
| 143 | GAAAUUAUCCCAAGCUGUAUA | UAUACAGCUUGGGAUAAUUUC | Chimp | [1444-1464] ORF |
| 144 | GAUUUACUACUGCCUCCAUCU | AGAUGGAGGCAGUAGUAAAUC | Chimp | [1386-1406] ORF |
| 145 | AUAGUAGGAGACUGGACAGAA | UUCUGUCCAGUCUCCUACUAU | Chimp | [1356-1376] ORF |
| 146 | CAUCUUAAAAUAGUAGGAGAC | GUCUCCUACUAUUUUAAGAUG | Chimp | [1347-1387] ORF |
| 147 | CCCUCAGAUGUCAUGGAAAUC | GAUUUCCAUGACAUCUGAGGG | Chimp | [1191-1211] ORF |
| 148 | CCAGAGUAUUUCUCAGAACAU | AUGUUCUGAGAAAUACUCUGG | Chimp | [957-977] ORF |
| 149 | UGACGUUGCAUGUUUCAGGAG | CUCCUGAAACAUGCAACGUCA | Chimp | [850-870] ORF |
| 150 | UCAUAACCUCUUCUUUGUCUU | AAGACAAAGAAGAGGUUAUGA | Chimp | [818-838] ORF |
| 151 | CUCCAUGAAUGUCCUGCUUUU | AAAAGCAGGACAUUCAUGGAG | Chimp | [308-328] ORF |
| 152 | ACCUGUGGUGUUACUAUCUGU | ACAGAUAGUAACACCACAGGU | Chimp | [558-578] ORF |
| 153 | GCAGAACAUUCCAUAUUACCU | AGGUAAUAUGGAAUGUUCUGC | Chimp | [541-581] ORF |
| 154 | GUUGAACAAAUGCAAGAUUGA | UCAAUCUUGCAUUUGUUCAAC | Chimp | [2339-2359] 3′UTR |
| 155 | CCCUGAAGAAUAUUUGAUGUG | CACAUCAAAUAUUCUUCAGGG | Chimp, Rat | [2172-2192] 3′UTR |
| 156 | GGAAUGAGUGCAAUUUCUAAG | CUUAGAAAUUGCACUCAUUCC | Chimp | [2009-2029] 3′UTR |
| 157 | GAAGGAAUGAGUGCAAUUUCU | AGAAAUUGCACUCAUUCCUUC | Chimp | [2008-2028] 3′UTR |
| 158 | CUCGGUGGAAACUUUUGUUUG | CAAACAAAAGUUUCCACCGAG | Chimp | [1810-1830] ORF |
| 159 | CAGCAUCUGUUCUUAACCUCA | UGAGGUUAAGAACAGAUGCUG | Chimp | [418-438] ORF |
| 160 | GGGAUACAGAAGAUAAUUGGA | UCCAAUUAUCUUCUGUAUCCC | Chimp, Rat | [1746-1766] ORF |
| 161 | CAGAUGGGAUACAGAAGAUAA | UUAUCUUCUGUAUCCCAUCUG | Chimp, Rat | [1741-1761] ORF |
| 162 | ACAGAUGGGAUACAGAAGAUA | UAUCUUCUGUAUCCCAUCUGU | Chimp, Rat | [1740-1760] ORF |
| 163 | UUAUUUGGGUAUGCAGAGAUA | UAUCUCUGCAUACCCAAAUAA | Chimp | [1615-1635] ORF |
| 164 | CUAUACUUUAUUUGGGUAUGC | GCAUACCCAAAUAAAGUAUAG | Chimp | [1608-1628] ORF |
| 165 | CCAUUUGCAUCAAUACUCAAC | GUUGAGUAUUGAUGCAAAUGG | Chimp | [1548-1568] ORF |
| 166 | CCCUUCAUUCAAUCUAGAAAU | AUUUCUAGAUUGAAUGAAGGG | Chimp | [1428-1448] ORF |
| 167 | UUACCCUCACAAUGUGUCCAA | UUGGACACAUUGUGAGGGUAA | Chimp | [1300-1320] ORF |
| 168 | CCAUCAUUUCGGUCAUAAGUC | GACUUAUGACCGAAAUGAUGG | Chimp | [1168-1188] ORF |
| 169 | GCUUUUCUGGAAAACCUUCUU | AAGAAGGUUUUCCAGAAAAGC | Chimp | [323-343] ORF |
| 170 | GAGAUGAGGAUCCUAGAAAAC | GUUUUCUAGGAUCCUCAUCUC | Chimp | [679-699] ORF |
| 171 | CUCUCCAUGAAUGUCCUGCUU | AAGCAGGACAUUCAUGGAGAG | Chimp | [306-326] ORF |
| 172 | CCAUAUUACCUGUGGUGUUAC | GUAACACCACAGGUAAUAUGG | Chimp | [551-571] ORF |
| 173 | GAACAUUCCAUAUUACCUGUG | CACAGGUAAUAUGGAAUGUUC | Chimp | [544-564] ORF |
| 174 | AGAACAUUCCAUAUUACCUGU | ACAGGUAAUAUGGAAUGUUCU | Chimp | [543-563] ORF |
| 175 | GGGCUUCCACUCAGUCUUUGA | UCAAAGACUGAGUGGAAGCCC | | [103-123] 5′UTR |
| 176 | GAACAAAUGCAAGAUUGAACA | UGUUCAAUCUUGCAUUUGUUC | Chimp | [2342-2362] 3′UTR |
| 177 | GUCGUGUUCCUCUUUAAAUUG | CAAUUUAAAGAGGAACACGAC | Chimp | [2312-2332] 3′UTR |
| 178 | GAUUGUCGUGUUCCUCUUUAA | UUAAAGAGGAACACGACAAUC | Chimp | [2308-2328] 3′UTR |
| 179 | CUGAUUGUCGUGUUCCUCUUU | AAAGAGGAACACGACAAUCAG | Chimp | [2306-2326] 3′UTR |
| 180 | CCUAGUGAGGAGCUGAACUUG | CAAGUUCAGCUCCUCACUAGG | Chimp | [2273-2293] 3′UTR |
| 181 | UGAUUUUCAUGGUGGAUUCAA | UUGAAUCCACCAUGAAAAUCA | | [2246-2266] 3′UTR |
| 182 | AUGCACACUGUUGAUUUUCAU | AUGAAAAUCAACAGUGUGCAU | | [2235-2255] 3′UTR |
| 183 | GGAAAUGUGAGAAUGUGGCAA | UUGCCACAUUCUCACAUUUCC | Chimp | [2109-2129] 3′UTR |
| 184 | AGGAAUGAGUGCAAUUUCUAA | UUAGAAAUUGCACUCAUUCCU | Chimp | [2008-2028] 3′UTR |
| 185 | CCUCAACUGCAGCCUUAUCCU | AGGAUAAGGCUGCAGUUGAGG | Chimp | [434-454] ORF |
| 186 | AUGAAGCAGGACUCUAAAGAA | UUCUUUAGAGUCCUGCUUCAU | Chimp | [1988-2008] 3′UTR |
| 187 | CCAUGAAGCAGGACUCUAAAG | CUUUAGAGUCCUGCUUCAUGG | Chimp | [1988-2008] 3′UTR |
| 188 | AGUAACCAGAACAACUCAUAU | AUAUGAGUUGUUCUGGUUACU | | [1917-1937] ORF |
| 189 | GUGUUUUCUGUUGUGGACCCA | UGGGUCCACAACAGAAAACAC | Chimp | [1867-1887] ORF |
| 190 | CAGAGGAAAAACAGUUGGUGU | ACACCAACUGUUUUUCCUCUG | | [1850-1870] ORF |
| 191 | ACAGAGGAAAAACAGUUGGUG | CACCAACUGUUUUUCCUCUGU | | [1849-1869] ORF |
| 192 | AUACUUUAUUUGGGUAUGCAG | CUGCAUACCCAAAUAAAGUAU | Chimp | [1610-1630] ORF |
| 193 | GCUGGAGGCAUUGGAGUAACU | AGUUACUCCAAUGCCUCCAGC | Chimp | [1527-1547] ORF |
| 194 | UGUAUAUUGAUGGUCCUUUUG | CAAAAGGACCAUCAAUAUACA | Chimp | [1459-1479] ORF |
| 195 | UGGACAGAACGAUUUCGAGAU | AUCUCGAAAUCGUUCUGUCCA | Chimp | [1368-1388] ORF |
| 196 | GAAAGACUUUACAGGUAUAUC | GAUAUACCUGUAAAGUCUUUC | Chimp | [1128-1148] ORF |
| 197 | AGACUUGGCUUUGGAUUUCUG | CAGAAAUCCAAAGCCAAGUCU | Chimp | [1084-1104] ORF |
| 198 | CUAACUAUGAUAUCUUCUGGU | ACCAGAAGAUAUCAUAGUUAG | Chimp | [793-813] ORF |
| 199 | GUGGUGGUGCUAUUCCUCAUG | CAUGAGGAAUAGCACCACCAC | Chimp | [741-761] ORF |
| 200 | UCUCCAUGAAUGUCCUGCUUU | AAAGCAGGACAUUCAUGGAGA | Chimp | [307-327] ORF |
| 201 | CAGUGAAGACUUUGUUGAACU | AGUUCAACAAAGUCUUCACUG | | [641-661] ORF |
| 202 | UACAGUGAAGACUUUGUUGAA | UUCAACAAAGUCUUCACUGUA | | [639-659] ORF |
| 203 | AAGCAGAACAUUCCAUAUUAC | GUAAUAUGGAAUGUUCUGCUU | Chimp | [539-559] ORF |
| 204 | GUUGAUUUUCAUGGUGGAUUC | GAAUCGACCAUGAAAAUCAAC | | [2244-2264] 3′UTR |
| 205 | ACUGUUGAUUUUCAUGGUGGA | UCCACCAUGAAAAUCAACAGU | | [2241-2261] 3′UTR |
| 206 | CACACUGUUGAUUUUCAUGGU | ACCAUGAAAAUCAACAGUGUG | | [2238-2258] 3′UTR |
| 207 | AGAUGCACACUGUUGAUUUUC | GAAAAUCAACAGUGUGCAUCU | | [2233-2253] 3′UTR |
| 208 | UUAGAUGCACACUGUUGAUUU | AAAUCAACAGUGUGCAUCUAA | Chimp, Rat | [2231-2251] 3′UTR |
| 209 | UGUUAGAUGCACACUGUUGAU | AUCAACAGUGUGCAUCUAACA | Chimp, Rat | [2229-2249] 3′UTR |
| 210 | AGACCCUGAAGAAUAUUUGAU | AUCAAAUAUUCUUCAGGGUCU | Chimp | [2169-2189] 3′UTR |
| 211 | GAGACCCUGAAGAAUAUUUGA | UCAAAUAUUCUUCAGGGUCUC | Chimp | [2168-2188] 3′UTR |
| 212 | UGGAAAUGUGAGAAUGUGGCA | UGCCACAUUCUCACAUUUCCA | Chimp | [2108-2128] 3′UTR |
| 213 | UCAACUGCAGCCUUAUCCUUU | AAAGGAUAAGGCUGCAGUUGA | Chimp | [436-456] ORF |
| 214 | GACUUUGAAACUCAGCGGAAU | AUUCCGCUGAGUUUCAAAGUC | Chimp | [2029-2049] 3′UTR |
| 215 | CAUAUGGGACAAGAUUUGAAU | AUUCAAAUCUUGUCCCAUAUG | | [1933-1953] ORF |
| 216 | UCAUAUGGGACAAGAUUUGAA | UUCAAAUCUUGUCCCAUAUGA | | [1932-1952] ORF |
| 217 | CUCAUAUGGGACAAGAUUUGA | UCAAAUCUUGUCCCAUAUGAG | | [1931-1951] ORF |
| 218 | UCCAAGACUCUUCAUAAACUG | CAGUUUAUGAAGAGUCUUGGA | Chimp | [1896-1916] ORF |
| 219 | UGUUUUCUGUUGUGGACCCAA | UUGGGUCCACAACAGAAAACA | Chimp | [1868-1888] ORF |
| 220 | GAGUUGGUGUUUUCUGUUGUG | CACAACAGAAAACACCAACUG | Chimp | [1861-1881] ORF |
| 221 | UCAAGACUGUUUAUAGGACGU | ACGUCCUAUAAACAGUCUUGA | | [1788-1808] ORF |
| 222 | GAUGGGAUACAGAAGAUAAUU | AAUUAUCUUCUGUAUCCCAUC | Chimp, Rat | [1743-1763] ORF |
| 223 | AGAUGGGAUACAGAAGAUAAU | AUUAUCUUCUGUAUCCCAUCU | Chimp, Rat | [1742-1762] ORF |
| 224 | CCAUCUAGUCAAGACUCCGAA | UUCGGAGUCUUGACUAGAUGG | Chimp | [1401-1421] ORF |
| 225 | ACCUGGUCAGUAUAUUACUCU | AGAGUAAUAUACUGACCAGGU | Chimp | [1241-1261] ORF |
| 226 | CAGACUUGGCUUUGGAUUUCU | AGAAAUCCAAAGCCAAGUCUG | Chimp | [1083-1103] ORF |
| 227 | UGAAGACUUUGUUGAACUGAA | UUCAGUUCAACAAAGUCUUCA | | [644-664] ORF |
| 228 | UCAUGGUGGAUUCAAGAACUC | GAGUUCUUGAAUCCACCAUGA | | [2252-2272] 3′UTR |
| 229 | UGCACACUGUUGAUUUUCAUG | CAUGAAAAUCAACAGUGUGCA | | [2236-2256] 3′UTR |
| 230 | ACCCUGAAGAAUAUUUGAUGU | ACAUCAAAUAUUCUUCAGGGU | Chimp | [2171-2191] 3′UTR |
| 231 | GACCCUGAAGAAUAUUUGAUG | CAUCAAAUAUUCUUCAGGGUC | Chimp | [2170-2190] 3′UTR |
| 232 | CUAUCCAAGACUCUUCAUAAA | UUUAUGAAGAGUCUUGGAUAG | Chimp | [1893-1913] ORF |
| 233 | ACUAUCCAAGACUCUUCAUAA | UUAUGAAGAGUCUUGGAUAGU | Chimp | [1892-1912] ORF |
| 234 | CACUAUCCAAGACUCUUCAUA | UAUGAAGAGUCUUGGAUAGUG | Chimp | [1891-1911] ORF |
| 235 | GUGUAUCUGCAUUAGAAAAUC | GAUUUUCUAAUGCAGAUACAC | Chimp | [1273-1293] ORF |
| 236 | CACAGACUUGGCUUUGGAUUU | AAAUCCAAAGCCAAGUCUGUG | Chimp | [1081-1101] ORF |
| 237 | GUGAAGACUUUGUUGAACUGA | UCAGUUCAACAAAGUCUUCAC | | [643-663] ORF |
| 238 | AUAUUACCUGUGGUGUUACUA | UAGUAACACCACAGGUAAUAU | Chimp | [553-573] ORF |
| 239 | UGUCGUGUUCCUCUUUAAAUU | AAUUUAAAGAGGAACACGACA | Chimp | [2311-2331] 3′UTR |
| 240 | UGAUUGUCGUGUUCCUCUUUA | UAAAGAGGAACACGACAAUCA | Chimp | [2307-2327] 3′UTR |
| 241 | CUAAGGCUGAUUGUCGUGUUC | GAACACGACAAUCAGCCUUAG | Chimp | [2300-2320] 3′UTR |
| 242 | CCUGAAGAAUAUUUGAUGUGA | UCACAUCAAAUAUUCUUCAGG | Chimp | [2173-2193] 3′UTR |
| 243 | GAAAUGUGAGAAUGUGGCAAG | CUUGCCACAUUCUCACAUUUC | Chimp | [2110-2130] 3′UTR |
| 244 | CUGAAAACUUUUGCCAUGAAG | CUUCAUGGCAAAAGUUUUCAG | Chimp | [1973-1993] |
| | | | | ORF + 3′UTR |
| 245 | GGAUACAGAAGAUAAUUGGAG | CUCCAAUUAUCUUCUGUAUCC | Chimp, Rat | [1747-1767] ORF |
| 246 | AACAGAUGGGAUACAGAAGAU | AUCUUCUGUAUCCCAUCUGUU | Chimp, Rat | [1739-1759] ORF |
| 247 | CUGGAGGCAUUGGAGUAACUC | GAGUUACUCCAAUGCCUCCAG | Chimp | [1528-1548] ORF |
| 248 | CCAGAGUAUCACUACCUCCAC | GUGGAGGUAGUGAUACUCUGG | Chimp | [360-380] ORF |
| 249 | CAGUGUAUCUGCAUUAGAAAA | UUUUCUAAUGCAGAUACACUG | Chimp | [1271-1291] ORF |
| 250 | GAAAUCCGAAUGGUCAAAGAA | UUCUUUGACCAUUCGGAUUUC | Chimp | [1206-1226] ORF |
| 251 | GGAAAUCCGAAUGGUCAAAGA | UCUUUGACCAUUCGGAUUUCC | Chimp | [1205-1225] ORF |
| 252 | CAAUAAGCCAGUCACCAUCAU | AUGAUGGUGACUGGCUUAUUG | Chimp | [1154-1174] ORF |
| 253 | CCAGCUCUCAGAAUAUUUCCU | AGGAAAUAUUCUGAGAGCUGG | Chimp | [934-954] ORF |
| 254 | GACUUUGUUGAACUGAAUGCA | UGCAUUCAGUUCAACAAAGUC | | [648-668] ORF |
| 255 | AGUGAAGACUUUGUUGAACUG | CAGUUCAACAAAGUCUUCACU | | [642-662] ORF |
| 256 | GCUCUCCAUGAAUGUCCUGCU | AGCAGGACAUUCAUGGAGAGC | Chimp | [305-325] ORF |
| 257 | GCUGAUUGUCGUGUUCCUCUU | AAGAGGAACACGACAAUCAGC | Chimp | [2305-2325] 3′UTR |
| 258 | AGGCUGAUUGUCGUGUUCCUC | GAGGAACACGACAAUCAGCCU | Chimp | [2303-2323] 3′UTR |
| 259 | UCUAAGGCUGAUUGUCGUGUU | AACACGACAAUCAGCCUUAGA | Chimp | [2299-2319] 3′UTR |
| 260 | ACCGUCACAUUACAUGUUUAA | UUAAACAUGUAAUGUGACGGU | | [2134-2154] 3′UTR |
| 261 | GUCAAACAGAUGGGAUACAGA | UCUGUAUCCCAUCUGUUUGAC | Chimp, Rat | [1735-1755] ORF |
| 262 | UCAGUCAAACAGAUGGGAUAC | GUAUCCCAUCUGUUUGACUGA | Chimp, Rat | [1732-1752] ORF |
| 263 | UCCUUUUGGAAGUCCAUUUGA | UCAAAUGGACUUCCAAAAGGA | Chimp | [1472-1492] ORF |
| 264 | CCAUUUACCCUCACAAUGUGU | ACACAUUGUGAGGGUAAAUGG | Chimp | [1296-1316] ORF |
| 265 | AAGCAAGACCUGGUCAGUAUA | UAUACUGACCAGGUCUUGCUU | Chimp | [1234-1254] ORF |
| 266 | GCAAUAAGCCAGUCACCAUCA | UGAUGGUGACUGGCUUAUUGC | Chimp | [1153-1173] ORF |
| 267 | CUGCUGAAGUAUCAAACUAAU | AUUAGUUUGAUACUUCAGCAG | Chimp | [873-893] ORF |
| 268 | GCUGCUGAAGUAUCAAACUAA | UUAGUUUGAUACUUCAGCAGC | Chimp | [872-892] ORF |
| 269 | ACUCAUAACCUCUUCUUUGUC | GACAAAGAAGAGGUUAUGAGU | Chimp | [816-836] ORF |
| 270 | GGAUAAAAGCAGAACAUUCCA | UGGAAUGUUCUGCUUUUAUCC | Chimp | [533-553] ORF |
| 271 | CAAGCAGGAGAACCAGGAGAU | AUCUCCUGGUUCUCCUGCUUG | Chimp | [506-526] ORF |
| 272 | AAGAUGACCGUCACAUUACAU | AUGUAAUGUGACGGUCAUCUU | | [2128-2148] 3′UTR |
| 273 | CAAGAUGACCGUCACAUUACA | UGUAAUGUGACGGUCAUCUUG | | [2127-2147] 3′UTR |
| 274 | UGUGAGAAUGUGGCAAGAUGA | UCAUCUUGCCACAUUCUCACA | Chimp | [2114-2134] 3′UTR |
| 275 | GAAAUUCUGCCCUUCAUUCAA | UUGAAUGAAGGGCAGAAUUUC | Chimp | [1419-1439] ORF |
| 276 | GGUGGUGCUAUUCCUCAUGAU | AUCAUGAGGAAUAGCACCACC | Chimp | [743-763] ORF |
| 277 | GGCUGAUUGUCGUGUUCCUCU | AGAGGAACACGACAAUCAGCC | Chimp | [2304-2324] 3′UTR |
| 278 | UCAAACAGAUGGGAUACAGAA | UUCUGUAUCCCAUCUGUUUGA | Chimp, Rat | [1736-1756] ORF |
| 279 | CUCAGUCAAACAGAUGGGAUA | UAUCCCAUCUGUUUGACUGAG | Chimp, Rat | [1731-1751] ORF |
| 280 | AGCAAGACCUGGUCAGUAUAU | AUAUACUGACCAGGUCUUGCU | Chimp | [1235-1255] ORF |
| 281 | CCUCAACUUCUCAGUGAAUUA | UAAUUCACUGAGAAGUUGAGG | Chimp | [620-640] ORF |
| 282 | CCCUCAACUUCUCAGUGAAUU | AAUUCACUGAGAAGUUGAGGG | Chimp | [619-639] ORF |
| 283 | CGAACACUCUUGGCUUACCUC | GAGGUAAGCCAAGAGUGUUCG | Chimp | [468-488] ORF |
| 284 | UCCAUUUGCAUCAAUACUCAA | UUGAGUAUUGAUGCAAAUGGA | Chimp | [1547-1567] ORF |
| 285 | GGCAUUGGAGUAACUCCAUUU | AAAUGGAGUUACUCCAAUGCC | Chimp | [1533-1553] ORF |
| 286 | UCCGAAAUUCUGCCCUUCAUU | AAUGAAGGGCAGAAUUUCGGA | Chimp | [1416-1436] ORF |
| 287 | CCAAGCUAAUUUUCCACAGAC | GUCUGUGGAAAAUUAGCUUGG | Chimp | [1067-1087] ORF |
| 288 | GGCUGCUGAAGUAUCAAACUA | UAGUUUGAUACUUCAGCAGCC | Chimp | [871-891] ORF |
| 289 | CUAGAAAACUUCUCUUCACAA | UUGUGAAGAGAAGUUUUCUAG | Chimp | [691-711] ORF |
| 290 | GCCCUCAACUUCUCAGUGAAU | AUUCACUGAGAAGUUGAGGGC | Chimp | [618-638] ORF |
| 291 | CCAUCUGGUGAAUGCCCUCAA | UUGAGGGCAUUCACCAGAUGG | Chimp | [605-625] ORF |
| 292 | GAAACCAAAGAGACCCUGAAG | CUUCAGGGUCUCUUUGGUUUC | Chimp | [2159-2179] 3′UTR |
| 293 | GCAUCAAUACUCAACACCCUG | CAGGGUGUUGAGUAUUGAUGC | | [1554-1574] ORF |
| 294 | CCCUCACAAUGUGUCCAACUG | CAGUUGGACACAUUGUGAGGG | Chimp | [1303-1323] ORF |
| 295 | GGUCAGUAUAUUACUCUACAU | AUGUAGAGUAAUAUACUGACC | Chimp | [1245-1265] ORF |
| 296 | GUCAUGGAAAUCCGAAUGGUC | GACCAUUCGGAUUUCCAUGAC | Chimp | [1200-1220] ORF |
| 297 | AGAGCCCAGAUUCCAAGCUAA | UUAGCUUGGAAUCUGGGCUCU | Chimp | [1055-1075] ORF |
| 298 | CAGAGUAUUUCUCAGAACAUU | AAUGUUCUGAGAAAUACUCUG | Chimp | [958-978] ORF |
| 299 | GUUGGAUAAAAGCAGAACAUU | AAUGUUCUGCUUUUAUCCAAC | Chimp | [530-550] ORF |
| 300 | UGUUGGAUAAAAGCAGAACAU | AUGUUCUGCUUUUAUCCAACA | Chimp | [529-549] ORF |
| 301 | AGAUUGUUGGAUAAAAGCAGA | UCUGCUUUUAUCCAACAAUCU | Chimp | [525-545] ORF |
| 302 | AACCAAAGAGACCCUGAAGAA | UUCUUCAGGGUCUCUUUGGUU | Chimp | [2161-2181] 3′UTR |
| 303 | GCCCUUCAUUCAAUCUAGAAA | UUUCUAGAUUGAAUGAAGGGC | Chimp, Rat | [1427-1447] ORF |
| 304 | UCACAAUGUGUCCAACUGAAA | UUUCAGUUGGACACAUUGUGA | Chimp | [1306-1326] ORF |
| 305 | GUCACCAUCAUUUCGGUCAUA | UAUGACCGAAAUGAUGGUGAC | Chimp | [1164-1184] ORF |
| 306 | CAAGCUAAUUUUCCACAGACU | AGUCUGUGGAAAAUUAGCUUG | Chimp | [1068-1088] ORF |
| 307 | GAAUAUUUCCUUACCAGAGUA | UACUCUGGUAAGGAAAUAUUC | Chimp | [944-964] ORF |
| 308 | UAACCUCUUCUUUGUCUUCUA | UAGAAGACAAAGAAGAGGUUA | Chimp | [821-841] ORF |
| 309 | GUGGUGCUAUUCCUCAUGAUC | GAUCAUGAGGAAUAGCACCAC | Chimp | [744-764] ORF |
| 310 | CCUAGAAAACUUCUCUUCACA | UGUGAAGAGAAGUUUUCUAGG | Chimp | [690-710] ORF |
| 311 | GAUUGUUGGAUAAAAGCAGAA | UUCUGCUUUUAUCCAACAAUC | Chimp | [526-546] ORF |
| 312 | GGAAACCAAAGAGACCCUGAA | UUCAGGGUCUCUUUGGUUUCC | Chimp | [2158-2178] 3′UTR |
| 313 | CUCCAUUUGCAUCAAUACUCA | UGAGUAUUGAUGCAAAUGGAG | Chimp | [1546-1566] ORF |
| 314 | GCCUCCAUCUAGUCAAGACUC | GAGUCUUGACUAGAUGGAGGC | Chimp | [1397-1417] ORF |
| 315 | CAAGACCUGGUCAGUAUAUUA | UAAUAUACUGACCAGGUCUUG | Chimp | [1237-1257] ORF |
| 316 | GAACCUUUCCCUGAAGGAUUU | AAAUCCUUCAGGGAAAGGUUC | Chimp | [984-1004] ORF |
| 317 | CCUCUUCUUUGUCUUCUACAU | AUGUAGAAGACAAAGAAGAGG | Chimp, Rat, Ms | [824-844] ORF |
| 318 | CCGAACACUCUUGGCUUACCU | AGGUAAGCCAAGAGUGUUCGG | Chimp | [467-487] ORF |
| 319 | ACCCAUGUGCCGAACACUCUU | AAGAGUGUUCGGCACAUGGGU | | [458-478] ORF |
| 320 | GGUUUGCAGAUUUACUCUGUA | UACAGAGUAAAUCUGCAAACC | Chimp | [1651-1671] ORF |
| 321 | GGUCCUUUUGGAAGUCCAUUU | AAAUGGACUUCCAAAAGGACC | Chimp | [1470-1490] ORF |
| 322 | UGCCUCCAUCUAGUCAAGACU | AGUCUUGACUAGAUGGAGGCA | Chimp | [1396-1416] ORF |
| 323 | GCAAGACCUGGUCAGUAUAUU | AAUAUACUGACCAGGUCUUGC | Chimp | [1236-1256] ORF |
| 324 | GUAUAACCAAGGGCCAGAGUA | UACUCUGGCCCUUGGUUAUAC | Chimp | [347-367] ORF |
| 325 | GCUUUGGAUUUCUGGACCUUU | AAAGGUCCAGAAAUCCAAAGC | Chimp | [1091-1111] ORF |
| 326 | CCCAGAUUCCAAGCUAAUUUU | AAAAUUAGCUUGGAAUCUGGG | Chimp | [1059-1079] ORF |
| 327 | CAGAGUUUACCCAGCACAAAU | AUUUGUGCUGGGUAAACUCUG | Chimp | [1015-1035] ORF |
| 328 | GGCAGAGUUUACCCAGCACAA | UUGUGCUGGGUAAACUCUGCC | Chimp | [1013-1033] ORF |
| 329 | GCAUCAGUCUUAACCGAACCA | UGGUUCGGUUAAGACUGAUGC | Chimp | [916-936] ORF |
| 330 | CCUGCUUUUCUGGAAAACCUU | AAGGUUUUCCAGAAAAGCAGG | Chimp, Rat | [320-340] ORF |
| 331 | CUCUUCUUUGUCUUCUACAUG | CAUGUAGAAGACAAAGAAGAG | Chimp, Rat, Ms | [825-845] ORF |
| 332 | ACCUCUUCUUUGUCUUCUACA | UGUAGAAGACAAAGAAGAGGU | Chimp, Rat, Ms | [823-843] ORF |
| 333 | UGCCCUCAACUUCUCAGUGAA | UUCACUGAGAAGUUGAGGGCA | Chimp | [617-637] ORF |
| 334 | GAACCAGGAGAUUGUUGGAUA | UAUCCAACAAUCUCCUGGUUC | Chimp | [517-537] ORF |
| 335 | GGAGCUGAACUUGCUCAAUCU | AGAUUGAGCAAGUUCAGCUCC | Chimp | [2281-2301] 3′UTR |
| 336 | UGAGGAGCUGAACUUGCUCAA | UUGAGCAAGUUCAGCUCCUCA | Chimp | [2278-2298] 3′UTR |
| 337 | CCAAAGAGACCCUGAAGAAUA | UAUUCUUCAGGGUCUCUUUGG | Chimp | [2163-2183] 3′UTR |
| 338 | CCGUCACAUUACAUGUUUAAU | AUUAAACAUGUAAUGUGACGG | | [2135-2155] 3′UTR |
| 339 | GCGGAAUCAAUCAGCUGUGUU | AACACAGCUGAUUGAUUCCGC | Chimp | [2043-2063] 3′UTR |
| 340 | UGGACCCAAUUCACUAUCCAA | UUGGAUAGUGAAUUGGGUCCA | Chimp | [1880-1900] ORF |
| 341 | ACGUCCUCGGUGGAAACUUUU | AAAAGUUUCCACCGAGGACGU | Chimp | [1805-1825] ORF |
| 342 | CUAUGUCAACAUCCAGCUGUA | UACAGCUGGAUGUUGACAUAG | Chimp | [1709-1729] ORF |
| 343 | GCAGAUUUACUCUGUAUGUUG | CAACAUACAGAGUAAAUCUGC | Chimp | [1656-1676] ORF |
| 344 | CUUCCGUUGGUUUGCAGAUUU | AAAUCUGCAAACCAACGGAAG | Chimp | [1643-1663] ORF |
| 345 | GCAUUGGAGUAACUCCAUUUG | CAAAUGGAGUUACUCCAAUGC | Chimp | [1534-1554] ORF |
| 346 | GUCCUUUUGGAAGUCCAUUUG | CAAAUGGACUUCCAAAAGGAC | Chimp | [1471-1491] ORF |
| 347 | ACUGAAACCAAAGCAACAUUU | AAAUGUUGCUUUGGUUUCAGU | Chimp, Rat, Ms | [1320-1340] ORF |
| 348 | CCUCACAAUGUGUCCAACUGA | UCAGUUGGACACAUUGUGAGG | Chimp | [1304-1324] ORF |
| 349 | CCAGUGUAUCUGCAUUAGAAA | UUUCUAAUGCAGAUACACUGG | Chimp | [1270-1290] ORF |
| 350 | CCCAGUGUAUCUGCAUUAGAA | UUCUAAUGCAGAUACACUGGG | Chimp | [1269-1289] ORF |
| 351 | GUCAUAAGUCAUCCCUCAGAU | AUCUGAGGGAUGACUUAUGAC | Chimp | [1179-1199] ORF |
| 352 | UCACCAUCAUUUCGGUCAUAA | UUAUGACCGAAAUGAUGGUGA | Chimp | [1165-1185] ORF |
| 353 | AGUCACCAUCAUUUCGGUCAU | AUGACCGAAAUGAUGGUGACU | Chimp | [1163-1183] ORF |
| 354 | CCAGUCACCAUCAUUUCGGUC | GACCGAAAUGAUGGUGACUGG | Chimp | [1161-1181] ORF |
| 355 | UCCAAGCUAAUUUUCCACAGA | UCUGUGGAAAAUUAGCUUGGA | Chimp | [1066-1086] ORF |
| 356 | GCAGAGUUUACCCAGCACAAA | UUUGUGCUGGGUAAACUCUGC | Chimp | [1014-1034] ORF |
| 357 | ACCAGAGUAUUUCUCAGAACA | UGUUCUGAGAAAUACUCUGGU | Chimp | [956-976] ORF |
| 358 | GCUGCUGACGUUGCAUGUUUC | GAAACAUGCAACGUCAGCAGC | Chimp | [845-865] ORF |
| 359 | CUUCUUUGUCUUCUACAUGCU | AGCAUGUAGAAGACAAAGAAG | Chimp, Rat, Ms | [827-847] ORF |
| 360 | CAGCCUCUACAUAUGCAAUAA | UUAUUGCAUAUGUAGAGGCUG | Chimp | [766-786] ORF |
| 361 | GUGCUAUUCCUCAUGAUCACA | UGUGAUCAUGAGGAAUAGCAC | Chimp | [747-767] ORF |
| 362 | GGUGGUGGUGCUAUUCCUCAU | AUGAGGAAUAGCACCACCACC | Chimp | [740-760] ORF |
| 363 | UCAACUUCUCAGUGAAUUACA | UGUAAUUCACUGAGAAGUUGA | Chimp | [822-842] ORF |
| 364 | GUGAAUGCCCUCAACUUCUCA | UGAGAAGUUGAGGGCAUUCAC | Chimp | [612-632] ORF |
| 365 | GAGGAGCUGAACUUGCUCAAU | AUUGAGCAAGUUCAGCUCCUC | Chimp | [2279-2299] 3′UTR |
| 366 | CUCAACACCCUGUUGGAUGAC | GUCAUCCAACAGGGUGUUGAG | | [1563-1583] ORF |
| 367 | UCAAUACUCAACACCCUGUUG | CAACAGGGUGUUGAGUAUUGA | | [1557-1577] ORF |
| 368 | UCUAGUCAAGACUCCGAAAUU | AAUUUCGGAGUCUUGACUAGA | Chimp | [1404-1424] ORF |
| 369 | CAUCUAGUCAAGACUCCGAAA | UUUCGGAGUCUUGACUAGAUG | Chimp | [1402-1422] ORF |
| 370 | AGAGUAUCACUACCUCCACCA | UGGUGGAGGUAGUGAUACUCU | Chimp | [362-382] ORF |
| 371 | AACUGAAACCAAAGCAACAUU | AAUGUUGCUUUGGUUUCAGUU | Chimp | [1319-1339] ORF |
| 372 | CGGUCAUAAGUCAUCCCUCAG | CUGAGGGAUGACUUAUGACCG | Chimp | [1177-1197] ORF |
| 373 | GCCCAGAUUCCAAGCUAAUUU | AAAUUAGCUUGGAAUCUGGGC | Chimp | [1058-1078] ORF |
| 374 | GAGCCCAGAUUCCAAGCUAAU | AUUAGCUUGGAAUCUGGGCUC | Chimp | [1058-1076] ORF |
| 375 | CCCUGAAGGAUUUUCAAAACC | GGUUUUGAAAAUCCUUCAGGG | Chimp | [992-1012] ORF |
| 376 | UGAACCUUUCCCUGAAGGAUU | AAUCCUUCAGGGAAAGGUUCA | Chimp | [983-1003] ORF |
| 377 | CUGGAAAACCUUCUUGCUGUA | UACAGCAAGAAGGUUUUCCAG | Chimp | [329-349] ORF |
| 378 | CUAAUUUAGAUACCCACCCUC | GAGGGUGGGUAUCUAAAUUAG | Chimp | [889-909] ORF |
| 379 | GGGCUGCUGAAGUAUCAAACU | AGUUUGAUACUUCAGCAGCCC | Chimp | [870-890] ORF |
| 380 | UGGUGGUGCUAUUCCUCAUGA | UCAUGAGGAAUAGCACCACCA | Chimp | [742-762] ORF |
| 381 | GGAUCCUAGAAAACUUCUCUU | AAGAGAAGUUUUCUAGGAUCC | Chimp | [686-706] ORF |
| 382 | GUGCCGAACACUCUUGGCUUA | UAAGCCAAGAGUGUUCGGCAC | Chimp | [464-484] ORF |
| 383 | GAGCUGAACUUGCUCAAUCUA | UAGAUUGAGCAAGUUCAGCUC | Chimp | [2282-2302] 3′UTR |
| 384 | GGUGGAUUCAAGAACUCCCUA | UAGGGAGUUCUUGAAUCCACC | Chimp | [2256-2276] 3′UTR |
| 385 | GUCUUUCAGCUGAAAACUUUU | AAAAGUUUUCAGCUGAAAGAC | Chimp | [1964-1984] |
| | | | | ORF + 3′UTR |
| 386 | UCAUAAACUGAGUAACCAGAA | UUCUGGUUACUCAGUUUAUGA | Chimp | [1907-1927] ORF |
| 387 | CAGACCUGACUAUGUCAACAU | AUGUUGACAUAGUCAGGUCUG | Chimp | [1700-1720] ORF |
| 388 | GCAAGAGAACAGACCUGACUA | UAGUCAGGUCUGUUCUCUUGC | Chimp | [1691-1711] ORF |
| 389 | UGCAGAUUUACUCUGUAUGUU | AACAUACAGAGUAAAUCUGCA | Chimp | [1655-1675] ORF |
| 390 | GUUUGCAGAUUUACUCUGUAU | AUACAGAGUAAAUCUGCAAAC | Chimp | [1652-1672] ORF |
| 391 | CCUUCCGUUGGUUUGCAGAUU | AAUCUGCAAACCAACGGAAGG | Chimp | [1642-1662] ORF |
| 392 | AGAUAUCCAGUCCUUCCGUUG | CAACGGAAGGACUGGAUAUCU | Chimp | [1631-1651] ORF |
| 393 | GGAAUCACUGAACUAUGAGGU | ACCUCAUAGUUCAGUGAUUCC | Chimp | [1493-1513] ORF |
| 394 | GAAGUCCAUUUGAGGAAUCAC | GUGAUUCCUCAAAUGGACUUC | Chimp | [1480-1500] ORF |
| 395 | CGAGAUUUACUACUGCCUCCA | UGGAGGCAGUAGUAAAUCUCG | Chimp, Rat | [1383-1403] ORF |
| 396 | GCCAGAGUAUCACUACCUCCA | UGGAGGUAGUGAUACUCUGGC | Chimp | [359-379] ORF |
| 397 | CUGCAUUAGAAAAUCAUCCAU | AUGGAUGAUUUUCUAAUGCAG | Chimp | [1279-1299] ORF |
| 398 | CAAGGGCCAGAGUAUCACUAC | GUAGUGAUACUCUGGCCCUUG | Chimp | [354-374] ORF |
| 399 | UCAUAAGUCAUCCCUCAGAUG | CAUCUGAGGGAUGACUUAUGA | Chimp | [1180-1200] ORF |
| 400 | CUGUAUAACCAAGGGCCAGAG | CUCUGGCCCUUGGUUAUACAG | Chimp | [345-365] ORF |
| 401 | GUACUGUGCCGAAAGACUUUA | UAAAGUCUUUCGGCACAGUAC | Chimp | [1118-1138] ORF |
| 402 | CUGUACUGUGCCGAAAGACUU | AAGUCUUUCGGCACAGUACAG | Chimp | [1118-1138] ORF |
| 403 | UGUGAAGAUUUGUAUGGAAGA | UCUUCCAUACAAAUCUUCACA | Chimp | [1037-1057] ORF |
| 404 | CAGCACAAAUUUGUGAAGAUU | AAUCUUCACAAAUUUGUGCUG | Chimp | [1026-1046] ORF |
| 405 | CCAGCACAAAUUUGUGAAGAU | AUCUUCACAAAUUUGUGCUGG | Chimp | [1025-1045] ORF |
| 406 | AGAGUUUACCCAGCACAAAUU | AAUUUGUGCUGGGUAAACUCU | Chimp | [1016-1036] ORF |
| 407 | AGGAUUUUCAAAACCGGCAGA | UCUGCCGGUUUUGAAAAUCCU | Chimp | [996-1016] ORF |
| 408 | UUACCAGAGUAUUUCUCAGAA | UUCUGAGAAAUACUCUGGUAA | Chimp | [954-974] ORF |
| 409 | UCCCGGCUGCAUCAGUCUUAA | UUAAGACUGAUGCAGCCGGGA | Chimp | [908-928] ORF |
| 410 | AGAAAACUUCUCUUCACAACU | AGUUGUGAAGAGAAGUUUUCU | Chimp | [693-713] ORF |
| 411 | AACCAGGAGAUUGUUGGAUAA | UUAUCCAACAAUCUCCUGGUU | Chimp | [518-538] ORF |
| 412 | CAGGAGAACCAGGAGAUUGUU | AACAAUCUCCUGGUUCUCCUG | Chimp | [512-532] ORF |
| 413 | UGGAAACCAAAGAGACCCUGA | UCAGGGUCUCUUUGGUUUCCA | Chimp | [2157-2177] ORF |
| 414 | AGCUGUGUUAUGCCAAAGAAU | AUUCUUUGGCAUAACACAGCU | Chimp | [2055-2075] ORF |
| 415 | GACUCUAAAGAAGGAAUGAGU | ACUCAUUCCUUCUUUAGAGUC | Chimp | [1997-2017] ORF |
| 416 | GCCAUGAAGCAGGACUCUAAA | UUUAGAGUCCUGCUUCAUGGC | Chimp | [1985-2005] ORF |
| 417 | GGACCCAAUUCACUAUCCAAG | CUUGGAUAGUGAAUUGGGUCC | Chimp | [1881-1901] ORF |
| 418 | GUGGACCCAAUUCACUAUCCA | UGGAUAGUGAAUUGGGUCCAC | Chimp | [1879-1899] ORF |
| 419 | GAGGAAAAACAGUUGGUGUUU | AAACACCAACUGUUUUUCCUC | | [1852-1872] ORF |
| 420 | GACGUCCUCGGUGGAAACUUU | AAAGUUUCCACCGAGGACGUC | Chimp | [1804-1824] ORF |
| 421 | GCACUGAAUUCAAGACUGUUU | AAACAGUCUUGAAUUCAGUGC | Chimp | [1779-1799] ORF |
| 422 | GAGCCUCAGCAUCUGUUCUUA | UAAGAACAGAUGCUGAGGCUC | Chimp | [412-432] ORF |
| 423 | CUGUAUGUUGCAUAACAAGUU | AACUUGUUAUGCAACAUACAG | Chimp | [1667-1687] ORF |
| 424 | GAGAUAUCCAGUCCUUCCGUU | AACGGAAGGACUGGAUAUCUC | Chimp | [1630-1650] ORF |
| 425 | GUCUAAGCAGAGCCUCAGCAU | AUGCUGAGGCUCUGCUUAGAC | Chimp | [403-423] ORF |
| 426 | GGAAGUCCAUUUGAGGAAUCA | UGAUUCCUCAAAUGGACUUCC | Chimp | [1479-1499] ORF |
| 427 | AGACUCCGAAAUUCUGCCCUU | AAGGGCAGAAUUUCGGAGUCU | Chimp | [1412-1432] ORF |
| 428 | GUAUCACUACCUCCACCAGAU | AUCUGGUGGAGGUAGUGAUAC | Chimp | [365-385] ORF |
| 429 | GAGAUUUACUACUGCCUCCAU | AUGGAGGCAGUAGUAAAUCUC | Chimp, Rat | [1384-1404] ORF |
| 430 | CAAUGUGUCCAACUGAAACCA | UGGUUUCAGUUGGACACAUUG | Chimp | [1309-1329] ORF |
| 431 | AGGGCCAGAGUAUCACUACCU | AGGUAGUGAUACUCUGGCCCU | Chimp | [356-376] ORF |
| 432 | CUCUACAUUGUCCCAGUGUAU | AUACACUGGGACAAUGUAGAG | Chimp | [1258-1278] ORF |
| 433 | AAGUCAUCCCUCAGAUGUCAU | AUGACAUCUGAGGGAUGACUU | Chimp | [1184-1204] ORF |
| 434 | GAGCAAUAAGCCAGUCACCAU | AUGGUGACUGGCUUAUUGCUC | Chimp | [1151-1171] ORF |
| 435 | CAGGUAUAUCCGGAGCAAUAA | UUAUUGCUCCGGAUAUACCUG | Chimp | [1139-1159] ORF |
| 436 | UGCUGCUGACGUUGCAUGUUU | AAACAUGCAACGUCAGCAGCA | Chimp | [844-864] ORF |
| 437 | GGUGCUAUUCCUCAUGAUCAC | GUGAUCAUGAGGAAUAGCACC | Chimp | [746-766] ORF |
| 438 | CUGCAUGGUGGUGGUGCUAUU | AAUAGCACCACCACCAUGCAG | Chimp | [734-754] ORF |
| 439 | ACCAGGAGAUUGUUGGAUAAA | UUUAUCCAACAAUCUCCUGGU | Chimp | [519-539] ORF |
| 440 | UUGCUCAAUCUAAGGCUGAUU | AAUCAGCCUUAGAUUGAGCAA | Chimp | [2291-2311] 3′UTR |
| 441 | CCCUAGUGAGGAGCUGAACUU | AAGUUCAGCUCCUCACUAGGG | Chimp | [2272-2292] 3′UTR |
| 442 | GACCGUCACAUUACAUGUUUA | UAAACAUGUAAUGUGACGGUC | | [2133-2153] 3′UTR |
| 443 | CAGCUGUGUUAUGCCAAAGAA | UUCUUUGGCAUAACACAGCUG | Chimp | [2054-2074] 3′UTR |
| 444 | GCCUCAGCAUCUGUUCUUAAC | GUUAAGAACAGAUGCUGAGGC | Chimp | [414-434] ORF |
| 445 | ACUCUGUAUGUUGCAUAACAA | UUGUUAUGCAACAUACAGAGU | Chimp | [1664-1684] ORF |
| 446 | GGAUGACUGGAAACCAUACAA | UUGUAUGGUUUCCAGUCAUCC | Chimp, Rat, Ms | [1577-1597] ORF |
| 447 | AGGCAUUGGAGUAACUCCAUU | AAUGGAGUUACUCCAAUGCCU | Chimp | [1532-1552] ORF |
| 448 | UUGGAAGUCCAUUUGAGGAAU | AUUCCUCAAAUGGACUUCCAA | Chimp | [1477-1497] ORF |
| 449 | GGGCUAGGAUUGUGUCUAAGC | GCUUAGACACAAUCCUAGCCC | Chimp | [390-410] ORF |
| 450 | CCCAAGCUGUAUAUUGAUGGU | ACCAUCAAUAUACAGCUUGGG | Chimp | [1452-1472] ORF |
| 451 | CUCACAAUGUGUCCAACUGAA | UUCAGUUGGACACAUUGUGAG | Chimp | [1305-1325] ORF |
| 452 | UGUCCCAGUGUAUCUGCAUUA | UAAUGCAGAUACACUGGGACA | Chimp | [1266-1286] ORF |
| 453 | AUCCGAAUGGUCAAAGAAAAU | AUUUUCUUUGACCAUUCGGAU | Chimp | [1209-1229] ORF |
| 454 | GUGCCGAAAGACUUUACAGGU | ACCUGUAAAGUCUUUCGGCAC | Chimp | [1123-1143] ORF |
| 455 | UCCUUACCAGAGUAUUUCUCA | UGAGAAAUACUCUGGUAAGGA | Chimp | [951-971] ORF |
| 456 | AGCUCUCAGAAUAUUUCCUUA | UAAGGAAAUAUUCUGAGAGCU | Chimp | [936-956] ORF |
| 457 | CCGAACCAGCUCUCAGAAUAU | AUAUUCUGAGAGCUGGUUCGG | Chimp | [929-949] ORF |
| 458 | CUGGUAUACUCAUAACCUCUU | AAGAGGUUAUGAGUAUACCAG | Chimp | [809-829] ORF |
| 459 | AGCCUCUACAUAUGCAAUAAG | CUUAUUGCAUAUGUAGAGGCU | Chimp | [767-787] ORF |
| 460 | ACAGCCUCUACAUAUGCAAUA | UAUUGCAUAUGUAGAGGCUGU | Chimp | [765-785] ORF |
| 461 | UCACAGCCUCUACAUAUGCAA | UUGCAUAUGUAGAGGCUGUGA | Chimp | [763-783] ORF |
| 462 | UCAGUGAAUUACAGUGAAGAC | GUCUUCACUGUAAUUCACUGA | | [630-650] ORF |
| 463 | GCAGGAGAACCAGGAGAUUGU | ACAAUCUCCUGGUUCUCCUGC | Chimp | [511-531] ORF |
| 464 | CCUGUUCAUCUGGCUCUCCAU | AUGGAGAGCCAGAUGAACAGG | | [293-313] ORF |
| 465 | GGAUUCAAGAACUCCCUAGUG | CACUAGGGAGUUCUUGAAUCC | Chimp | [2259-2279] 3′UTR |
| 466 | ACCAAAGAGACCCUGAAGAAU | AUUCUUCAGGGUCUCUUUGGU | Chimp | [2162-2182] 3′UTR |
| 467 | UGACCGUCACAUUACAUGUUU | AAACAUGUAAUGUGACGGUCA | | [2132-2152] 3′UTR |
| 468 | CAAUCAGCUGUGUUAUGCCAA | UUGGCAUAACACAGCUGAUUG | Chimp | [2050-2070] 3′UTR |
| 469 | UCAGCUGAAAACUUUUGCCAU | AUGGCAAAAGUUUUCAGCUGA | Chimp | [1969-1989] |
| | | | | ORF + 3′UTR |
| 470 | AGAGUCUUUCAGCUGAAAACU | AGUUUUCAGCUGAAAGACUCU | Chimp | [1961-1981] |
| | | | | ORF + 3′UTR |
| 471 | UAAACUGAGUAACCAGAACAA | UUGUUCUGGUUACUCAGUUUA | | [1910-1930] ORF |
| 472 | GAGAAAAAUAUCAUGCACUGA | UCAGUGCAUGAUAUUUUUCUC | Chimp | [1765-1785] ORF |
| 473 | CAGCUGUACCUCAGUCAAACA | UGUUUGACUGAGGUACAGCUG | Chimp, Rat, Ms | [1722-1742] ORF |
| 474 | UUCCGUUGGUUUGCAGAUUUA | UAAAUCUGCAAACCAACGGAA | Chimp | [1644-1664] ORF |
| 475 | AGAGAUAUCCAGUCCUUCCGU | ACGGAAGGACUGGAUAUCUCU | Chimp | [1629-1649] ORF |
| 476 | GCUAGGAUUGUGUCUAAGCAG | CUGCUUAGACACAAUCCUAGC | Chimp | [392-412] ORF |
| 477 | GGCUAGGAUUGUGUCUAAGCA | UGCUUAGACACAAUCCUAGCC | Chimp | [391-411] ORF |
| 478 | CCAAGCUGUAUAUUGAUGGUC | GACCAUCAAUAUACAGCUUGG | Chimp | [1453-1473] ORF |
| 479 | CAAUCUAGAAAUUAUCCCAAG | CUUGGGAUAAUUUCUAGAUUG | Chimp | [1437-1457] ORF |
| 480 | UCACUACCUCCACCAGAUGUU | AACAUCUGGUGGAGGUAGUGA | Chimp | [368-388] ORF |
| 481 | CUCCGAAAUUCUGCCCUUCAU | AUGAAGGGCAGAAUUUCGGAG | Chimp | [1415-1435] ORF |
| 482 | GAGUAUCACUACCUCCACCAG | CUGGUGGAGGUAGUGAUACUC | Chimp | [363-383] ORF |
| 483 | CAACUGAAACCAAAGCAACAU | AUGUUGCUUUGGUUUCAGUUG | Chimp | [1318-1338] ORF |
| 484 | CCAACUGAAACCAAAGCAACA | UGUUGCUUUGGUUUCAGUUGG | Chimp | [1317-1337] ORF |
| 485 | GUCCCAGUGUAUCUGCAUUAG | CUAAUGCAGAUACACUGGGAC | Chimp | [1267-1287] ORF |
| 486 | GGAGCAAUAAGCCAGUCACCA | UGGUGACUGGCUUAUUGCUCC | Chimp | [1150-1170] ORF |
| 487 | GGCUUUGGAUUUCUGGACCUU | AAGGUCCAGAAAUCCAAAGCC | Chimp | [1090-1110] ORF |
| 488 | CCUGAAGGAUUUUCAAAACCG | CGGUUUUGAAAAUCCUUCAGG | Chimp | [993-1013] ORF |
| 489 | GCUCUCAGAAUAUUUCCUUAC | GUAAGGAAAUAUUCUGAGAGC | Chimp | [937-957] ORF |
| 490 | GUCUUAACCGAACCAGCUCUC | GAGAGCUGGUUCGGUUAAGAC | Chimp | [922-942] ORF |
| 491 | GCUGCAUCAGUCUUAACCGAA | UUCGGUUAAGACUGAUGCAGC | Chimp | [913-933] ORF |
| 492 | CUGCUGACGUUGCAUGUUUCA | UGAAACAUGCAACGUCAGCAG | Chimp | [846-866] ORF |
| 493 | CCGAGAUGAGGAUCCUAGAAA | UUUCUAGGAUCCUCAUCUCGG | Chimp | [677-697] ORF |
| 494 | CUCAGUGAAUUACAGUGAAGA | UCUUCACUGUAAUUCACUGAG | | [629-649] ORF |
| 495 | UGCCGAACACUCUUGGCUUAC | GUAAGCCAAGAGUGUUCGGCA | Chimp | [465-485] ORF |
| 496 | GAACUUGCUCAAUCUAAGGCU | AGCCUUAGAUUGAGCAAGUUC | Chimp | [2287-2307] 3′UTR |
| 497 | GCUGAACUUGCUCAAUCUAAG | CUUAGAUUGAGCAAGUUCAGC | Chimp | [2284-2304] 3′UTR |
| 498 | AGCUGAACUUGCUCAAUCUAA | UUAGAUUGAGCAAGUUCAGCU | Chimp | [2283-2303] 3′UTR |
| 499 | UGAUGUGAUGAUUCACUUUUC | GAAAAGUGAAUCAUCACAUCA | Chimp | [2186-2206] 3′UTR |
| 500 | UAACCUCAACUGCAGCCUUAU | AUAAGGCUGCAGUUGAGGUUA | Chimp | [431-451] ORF |
| 501 | UAAACACCUCUGCCUGUUCAU | AUGAACAGGCAGAGGUGUUUA | | [281-301] ORF |
| 502 | GAGUAACCAGAACAACUCAUA | UAUGAGUUGUUCUGGUUACUC | | [1916-1936] ORF |
| 503 | UGCACUGAAUUCAAGACUGUU | AACAGUCUUGAAUUCAGUGCA | Chimp | [1778-1798] ORF |
| 504 | CCUCAGCAUCUGUUCUUAACC | GGUUAAGAACAGAUGCUGAGG | Chimp | [415-435] ORF |
| 505 | AGCCUCAGCAUCUGUUCUUAA | UUAAGAACAGAUGCUGAGGCU | Chimp | [413-433] ORF |
| 506 | CCAGCUGUACCUCAGUCAAAC | GUUUGACUGAGGUACAGCUGG | Chimp, Rat, Ms | [1721-1741] ORF |
| 507 | UCCGUUGGUUUGCAGAUUUAC | GUAAAUCUGCAAACCAACGGA | Chimp | [1645-1665] ORF |
| 508 | AUCCAGUCCUUCCGUUGGUUU | AAACCAACGGAAGGACUGGAU | Chimp | [1635-1655] ORF |
| 509 | ACCCUGUUGGAUGACUGGAAA | UUUCCAGUCAUCCAACAGGGU | | [1569-1589] ORF |
| 510 | CAACACCCUGUUGGAUGACUG | CAGUCAUCCAACAGGGUGUUG | | [1565-1585] ORF |
| 511 | AUAUUGAUGGUCCUUUUGGAA | UUCCAAAAGGACCAUCAAUAU | Chimp | [1462-1482] ORF |
| 512 | CUAGAAAUUAUCCCAAGCUGU | ACAGCUUGGGAUAAUUUCUAG | Chimp | [1441-1461] ORF |
| 513 | CCGAAAUUCUGCCCUUCAUUC | GAAUGAAGGGCAGAAUUUCGG | Chimp | [1417-1437] ORF |
| 514 | CUGAAACCAAAGCAACAUUUG | CAAAUGUUGCUUUGGUUUCAG | Chimp, Rat, Ms | [1321-1341] ORF |
| 515 | AGAUGUCAUGGAAAUCCGAAU | AUUCGGAUUUCCAUGACAUCU | Chimp | [1196-1216] ORF |
| 516 | UCCGGAGCAAUAAGCCAGUCA | UGACUGGCUUAUUGCUCCGGA | Chimp | [1147-1167] ORF |
| 517 | CCACAGACUUGGCUUUGGAUU | AAUCCAAAGCCAAGUCUGUGG | Chimp | [1080-1100] ORF |
| 518 | AGCCCAGAUUCCAAGCUAAUU | AAUUAGCUUGGAAUCUGGGCU | Chimp | [1057-1077] ORF |
| 519 | GAACAUUUUCAUGAACCUUUC | GAAAGGUUCAUGAAAAUGUUC | Chimp | [972-992] ORF |
| 520 | UCUCAGAACAUUUUCAUGAAC | GUUCAUGAAAAUGUUCUGAGA | Chimp | [967-987] ORF |
| 521 | UAACCGAACCAGCUCUCAGAA | UUCUGAGAGCUGGUUCGGUUA | Chimp | [926-946] ORF |
| 522 | GCCUCUACAUAUGCAAUAAGA | UCUUAUUGCAUAUGUAGAGGC | Chimp | [768-788] ORF |
| 523 | UCUCAGUGAAUUACAGUGAAG | CUUCACUGUAAUUCACUGAGA | | [628-648] ORF |
| 524 | UUCUCAGUGAAUUACAGUGAA | UUCACUGUAAUUCACUGAGAA | | [627-647] ORF |
| 525 | UCCAUAUUACCUGUGGUGUUA | UAACACCACAGGUAAUAUGGA | Chimp | [550-570] ORF |
| 526 | AGAAGGUUCCAAGCAGGAGAA | UUCUCCUGCUUGGAACCUUCU | Chimp | [499-519] ORF |
| 527 | UGGUGGAUUCAAGAACUCCCU | AGGGAGUUCUUGAAUCCACCA | Chimp | [2255-2275] 3′UTR |
| 528 | CAUGUUUAAUCUGGAAACCAA | UUGGUUUCCAGAUUAAACAUG | Chimp | [2146-2166] 3′UTR |
| 529 | CCAAGACUCUUCAUAAACUGA | UCAGUUUAUGAAGAGUCUUGG | Chimp | [1897-1917] ORF |
| 530 | UCCUCGGUGGAAACUUUUGUU | AACAAAAGUUUCCACCGAGGA | Chimp | [1808-1828] ORF |
| 531 | UCAGCAUCUGUUCUUAACCUC | GAGGUUAAGAACAGAUGCUGA | Chimp | [417-437] ORF |
| 532 | CUCUGUAUGUUGCAUAACAAG | CUUGUUAUGCAACAUACAGAG | Chimp | [1665-1685] ORF |
| 533 | CCGUUGGUUUGCAGAUUUACU | AGUAAAUCUGCAAACCAACGG | Chimp | [1646-1666] ORF |
| 534 | CCCUGUUGGAUGACUGGAAAC | GUUUCCAGUCAUCCAACAGGG | | [1570-1590] ORF |
| 535 | GUUAAACACCUCUGCCUGUU | AACAGGCAGAGGUGUUUAACC | | [278-298] ORF |
| 536 | GGAGUAACUCCAUUUGCAUCA | UGAUGCAAAUGGAGUUACUCC | Chimp | [1539-1559] ORF |
| 537 | GAGGCAUUGGAGUAACUCCAU | AUGGAGUUACUCCAAUGCCUC | Chimp | [1531-1551] ORF |
| 538 | GAAUCACUGAACUAUGAGGUC | GACCUCAUAGUUCAGUGAUUC | Chimp | [1494-1514] ORF |
| 539 | AGAAAUUAUCCCAAGCUGUAU | AUACAGCUUGGGAUAAUUUCU | Chimp | [1443-1463] ORF |
| 540 | GACUCCGAAAUUCUGCCCUUC | GAAGGGCAGAAUUUCGGAGUC | Chimp | [1413-1433] ORF |
| 541 | UCCCAGUGUAUCUGCAUUAGA | UCUAAUGCAGAUACACUGGGA | Chimp | [1268-1288] ORF |
| 542 | UGGUCAGUAUAUUACUCUACA | UGUAGAGUAAUAUACUGACCA | Chimp | [1244-1264] ORF |
| 543 | CUGGUCAGUAUAUUACUCUAC | GUAGAGUAAUAUACUGACCAG | Chimp | [1243-1263] ORF |
| 544 | UCCACAGACUUGGCUUUGGAU | AUCCAAAGCCAAGUCUGUGGA | Chimp | [1079-1099] ORF |
| 545 | CCUUUCCCUGAAGGAUUUUGA | UGAAAAUCCUUCAGGGAAAGG | Chimp | [987-1007] ORF |
| 546 | ACUAAUUUAGAUACCCACCCU | AGGGUGGGUAUCUAAAUUAGU | Chimp | [888-908] ORF |
| 547 | AGGGCUGCUGAAGUAUCAAAC | GUUUGAUACUUCAGCAGCCCU | Chimp | [869-889] ORF |
| 548 | CACAGCCUCUACAUAUGCAAU | AUUGCAUAUGUAGAGGCUGUG | Chimp | [764-784] ORF |
| 549 | UGAGGAUCCUAGAAAACUUCU | AGAAGUUUUCUAGGAUCCUCA | Chimp | [683-703] ORF |
| 550 | GCAGCAAGAUACCGAGAUGAG | CUCAUCUCGGUAUCUUGCUGC | Chimp | [666-686] ORF |
| 551 | UGAACUGAAUGCAGCAAGAUA | UAUCUUGCUGCAUUCAGUUCA | Chimp, Ms | [656-676] ORF |
| 552 | ACAUUCCAUAUUACCUGUGGU | ACCACAGGUAAUAUGGAAUGU | Chimp | [546-566] ORF |
| 553 | ACACUGUUGAUUUUCAUGGUG | CACCAUGAAAAUCAACAGUGU | | [2239-2259] 3′UTR |
| 554 | UCAGCUGUGUUAUGCCAAAGA | UCUUUGGCAUAACACAGCUGA | Chimp | [2053-2073] 3′UTR |
| 555 | ACAGUUGGUGUUUUCUGUUGU | ACAACAGAAAACACCAACUGU | Chimp | [1860-1880] ORF |
| 556 | AAACAGUUGGUGUUUUCUGUU | AACAGAAAACACCAACUGUUU | Chimp | [1858-1878] ORF |
| 557 | AGACUGUUUAUAGGACGUCCU | AGGACGUCCUAUAAACAGUCU | | [1791-1811] ORF |
| 558 | CAAGAGAACAGACCUGACUAU | AUAGUCAGGUCUGUUCUCUUG | Chimp | [1692-1712] ORF |
| 559 | GUUGGAUGACUGGAAACCAUA | UAUGGUUUCCAGUCAUCCAAC | Chimp | [1574-1594] ORF |
| 560 | UUGGAGUAACUCCAUUUGCAU | AUGCAAAUGGAGUUACUCCAA | Chimp | [1537-1557] ORF |
| 561 | UGGAAGUCCAUUUGAGGAAUC | GAUUCCUCAAAUGGACUUCCA | Chimp | [1478-1498] ORF |
| 562 | AUGGUCCUUUUGGAAGUCCAU | AUGGACUUCCAAAAGGACCAU | Chimp | [1468-1488] ORF |
| 563 | GGGUUAAACACCUCUGCCUGU | ACAGGCAGAGGUGUUUAACCC | | [277-297] ORF |
| 564 | UCCAACUGAAACCAAAGCAAC | GUUGCUUUGGUUUCAGUUGGA | Chimp | [1316-1336] ORF |
| 565 | CUACAUUGUCCCAGUGUAUCU | AGAUACACUGGGACAAUGUAG | Chimp | [1260-1280] ORF |
| 566 | CACCAUCAUUUCGGUCAUAAG | CUUAUGACCGAAAUGAUGGUG | Chimp | [1166-1186] ORF |
| 567 | UACAGGUAUAUCCGGAGCAAU | AUUGCUCCGGAUAUACCUGUA | Chimp | [1137-1157] ORF |
| 568 | UGCCGAAAGACUUUACAGGUA | UACCUGUAAAGUCUUUCGGCA | Chimp | [1124-1144] ORF |
| 569 | GCACAAAUUUGUGAAGAUUUG | CAAAUCUUCACAAAUUUGUGC | Chimp | [1028-1048] ORF |
| 570 | UGGAAAACCUUCUUGCUGUAU | AUACAGCAAGAAGGUUUUCCA | Chimp | [330-350] ORF |
| 571 | AACCUUUCCCUGAAGGAUUUU | AAAAUCCUUCAGGGAAAGGUU | Chimp | [985-1005] ORF |
| 572 | CUGACGUUGCAUGUUUCAGGA | UCCUGAAACAUGCAACGUCAG | Chimp | [849-869] ORF |
| 573 | UGGUAUACUCAUAACCUCUUC | GAAGAGGUUAUGAGUAUACCA | Chimp | [810-830] ORF |
| 574 | AGGAUCCUAGAAAACUUCUCU | AGAGAAGUUUUCUAGGAUCCU | Chimp | [685-705] ORF |
| 575 | UGCAGCAAGAUACCGAGAUGA | UCAUCUCGGUAUCUUGCUGCA | Chimp | [665-685] ORF |
| 576 | UUGAACUGAAUGCAGCAAGAU | AUCUUGCUGCAUUCAGUUCAA | Chimp, Ms | [655-675] ORF |
| 577 | UUCAUCUGGCUCUCCAUGAAU | AUUCAUGGAGAGCCAGAUGAA | Chimp | [297-317] ORF |
| 578 | GCUCAAUCUAAGGCUGAUUGU | ACAAUCAGCCUUAGAUUGAGC | Chimp | [2293-2313] 3′UTR |
| 579 | UGGAUUCAAGAACUCCCUAGU | ACUAGGGAGUUCUUGAAUCCA | Chimp | [2258-2278] 3′UTR |
| 580 | AACUGUUAGAUGCACACUGUU | AACAGUGUGCAUCUAACAGUU | Chimp | [2226-2246] 3′UTR |
| 581 | GAUGAUUCACUUUUCAGUUCU | AGAACUGAAAAGUGAAUCAUC | Chimp | [2192-2212] 3′UTR |
| 582 | GAUGUGAUGAUUCACUUUUCA | UGAAAAGUGAAUCAUCACAUC | Chimp | [2187-2207] 3′UTR |
| 583 | UGGCAAGAUGACCGUCACAUU | AAUGUGACGGUCAUCUUGCCA | | [2124-2144] 3′UTR |
| 584 | AGAAUGUGGCAAGAUGACCGU | ACGGUCAUCUUGCCACAUUCU | | [2118-2138] 3′UTR |
| 585 | AGCGGAAUCAAUCAGCUGUGU | ACACAGCUGAUUGAUUCCGCU | Chimp | [2042-2062] 3′UTR |
| 586 | AAAGAGUCUUUCAGCUGAAAA | UUUUCAGCUGAAAGACUCUUU | Chimp | [1959-1979] |
| | | | | ORF + 3′UTR |
| 587 | UAAAGAGUCUUUCAGCUGAAA | UUUCAGCUGAAAGACUCUUUA | Chimp | [1958-1978] |
| | | | | ORF + 3′UTR |
| 588 | AGACUCUUCAUAAACUGAGUA | UACUCAGUUUAUGAAGAGUCU | Chimp | [1900-1920] ORF |
| 589 | GGAAAAACAGUUGGUGUUUUC | GAAAACACCAACUGUUUUUCC | | [1854-1874] ORF |
| 590 | GACUGUUUAUAGGACGUCCUC | GAGGACGUCCUAUAAACAGUC | | [1792-1812] ORF |
| 591 | CCUGACUAUGUCAACAUCCAG | CUGGAUGUUGACAUAGUCAGG | Chimp | [1704-1724] ORF |
| 592 | AGAACAGACCUGACUAUGUCA | UGACAUAGUCAGGUCUGUUCU | Chimp | [1696-1716] ORF |
| 593 | UUGCAUAACAAGUUUUGGCAA | UUGCCAAAACUUGUUAUGCAA | Chimp, Ms | [1674-1694] ORF |
| 594 | UGGUUUGCAGAUUUACUCUGU | ACAGAGUAAAUCUGCAAACCA | Chimp, Ms | [1650-1670] ORF |
| 595 | GUCCUUCCGUUGGUUUGCAGA | UCUGCAAACCAACGGAAGGAC | Chimp | [1640-1660] ORF |
| 596 | GAGGAAUCACUGAACUAUGAG | CUCAUAGUUCAGUGAUUCCUC | Chimp | [1491-1511] ORF |
| 597 | AGACCUGGUCAGUAUAUUACU | AGUAAUAUACUGACCAGGUCU | Chimp | [1239-1259] ORF |
| 598 | ACAGGUAUAUCCGGAGCAAUA | UAUUGCUCCGGAUAUACCUGU | Chimp | [1138-1158] ORF |
| 599 | UGGACCUUUGUGCCUGUACUG | CAGUACAGGCACAAAGGUCCA | Chimp | [1103-1123] ORF |
| 600 | CUUUCCCUGAAGGAUUUUCAA | UUGAAAAUCCUUCAGGGAAAG | Chimp | [968-1008] ORF |
| 601 | CAGCUCUCAGAAUAUUUCCUU | AAGGAAAUAUUCUGAGAGCUG | Chimp | [935-955] ORF |
| 602 | CGAACCAGCUCUCAGAAUAUU | AAUAUUCUGAGAGCUGGUUCG | Chimp | [930-950] ORF |
| 603 | GGUAUACUCAUAACCUCUUCU | AGAAGAGGUUAUGAGUAUACC | Chimp | [811-831] ORF |
| 604 | CCUCUACAUAUGCAAUAAGAG | CUCUUAUUGCAUAUGUAGAGG | Chimp | [769-789] ORF |
| 605 | ACCGAGAUGAGGAUCCUAGAA | UUCUAGGAUCCUCAUCUCGGU | Chimp | [676-696] ORF |
| 606 | AGAUACCGAGAUGAGGAUCCU | AGGAUCCUCAUCUCGGUAUCU | Chimp | [672-692] ORF |
| 607 | GAAGGUUCCAAGCAGGAGAAC | GUUCUCCUGCUUGGAACCUUC | Chimp | [500-520] ORF |
| 608 | GAAAACUGUUAGAUGCACACU | AGUGUGCAUCUAACAGUUUUC | Chimp | [2223-2243] 3′UTR |
| 609 | GUGAUGAUUCACUUUUCAGUU | AACUGAAAAGUGAAUCAUCAC | Chimp | [2190-2210] 3′UTR |
| 610 | CGUCACAUUACAUGUUUAAUC | GAUUAAACAUGUAAUGUGACG | | [2136-2156] 3′UTR |
| 611 | GGCAAGAUGACCGUCACAUUA | UAAUGUGACGGUCAUCUUGCC | | [2125-2145] 3′UTR |
| 612 | CAGCGGAAUCAAUCAGCUGUG | CACAGCUGAUUGAUUCCGCUG | Chimp | [2041-2061] 3′UTR |
| 613 | UGAGUAACCAGAACAACUCAU | AUGAGUUGUUCUGGUUACUCA | | [1915-1935] ORF |
| 614 | CUGAGUAACCAGAACAACUCA | UGAGUUGUUCUGGUUACUCAG | | [1914-1934] ORF |
| 615 | GCUGUACCUCAGUCAAACAGA | UCUGUUUGACUGAGGUACAGC | Chimp, Rat, Ms | [1724-1744] ORF |
| 616 | AGACCUGACUAUGUCAACAUC | GAUGUUGACAUAGUCAGGUCU | Chimp | [1701-1721] ORF |
| 617 | GAACAGACCUGACUAUGUCAA | UUGACAUAGUCAGGUCUGUUC | Chimp | [1697-1717] ORF |
| 618 | GGCAAGAGAACAGACCUGACU | AGUCAGGUCUGUUCUCUUGCC | Chimp | [1690-1710] ORF |
| 619 | CACCCUGUUGGAUGACUGGAA | UUCCAGUCAUCCAACAGGGUG | | [1568-1588] ORF |
| 620 | UCGGGCUUCCACUCAGUCUUU | AAAGACUGAGUGGAAGCCCGA | | [101-121] 5′UTR |
| 621 | CUGAACUAUGAGGUCAGCCUC | GAGGCUGACCUCAUAGUUCAG | Chimp | [1500-1520] ORF |
| 622 | UCCAUUUGAGGAAUCACUGAA | UUCAGUGAUUCCUCAAAUGGA | Chimp | [1484-1504] ORF |
| 623 | GGACCUUUGUGCCUGUACUGU | ACAGUACAGGCACAAAGGUCC | Chimp | [1104-1124] ORF |
| 624 | GGAUUUCUGGACCUUUGUGCC | GGCACAAAGGUCCAGAAAUCC | Chimp, Rat, Ms | [1096-1116] ORF |
| 625 | CCCAGCACAAAUUUGUGAAGA | UCUUCACAAAUUUGUGCUGGG | Chimp | [1024-1044] ORF |
| 626 | GGCUGCAUCAGUCUUAACCGA | UCGGUUAAGACUGAUGCAGCC | Chimp | [912-932] ORF |
| 627 | UGCAUGGUGGUGGUGCUAUUC | GAAUAGCACCACCACCAUGCA | Chimp | [735-755] ORF |
| 628 | CAAGAUACCGAGAUGAGGAUC | GAUCCUCAUCUCGGUAUCUUG | Chimp | [670-690] ORF |
| 629 | GAGAACCAGGAGAUUGUUGGA | UCCAACAAUCUCCUGGUUCUC | Chimp | [515-535] ORF |
| 630 | UGCUCAAUCUAAGGCUGAUUG | CAAUCAGCCUUAGAUUGAGCA | Chimp | [2292-2312] 3′UTR |
| 631 | AAACUGUUAGAUGCACACUGU | ACAGUGUGCAUCUAACAGUUU | Chimp | [2225-2245] 3′UTR |
| 632 | GCAAGAUGACCGUCACAUUAC | GUAAUGUGACGGUCAUCUUGC | | [2126-2146] 3′UTR |
| 633 | ACUUUGAAACUCAGCGGAAUC | GAUUCCGCUGAGUUUCAAAGU | Chimp | [2030-2050] 3′UTR |
| 634 | UGCCAUGAAGCAGGACUCUAA | UUAGAGUCCUGCUUCAUGGCA | Chimp | [1984-2004] 3′UTR |
| 635 | AGCUGAAAACUUUUGCCAUGA | UCAUGGCAAAAGUUUUCAGCU | Chimp | [1971-1991] |
| | | | | ORF + 3′UTR |
| 636 | GACUCUUCAUAAACUGAGUAA | UUACUCAGUUUAUGAAGAGUC | Chimp | [1901-1921] ORF |
| 637 | CCUCAGUCAAACAGAUGGGAU | AUCCCAUCUGUUUGACUGAGG | Chimp, Rat, Ms | [1730-1750] ORF |
| 638 | UGACUAUGUCAACAUCCAGCU | AGCUGGAUGUUGACAUAGUCA | Chimp | [1706-1726] ORF |
| 639 | UGGCAAGAGAACAGACCUGAC | GUCAGGUCUGUUCUCUUGCCA | Chimp | [1689-1709] ORF |
| 640 | GGAGGCAUUGGAGUAACUCCA | UGGAGUUACUCCAAUGCCUCC | Chimp | [1530-1550] ORF |
| 641 | UGAGGAAUCACUGAACUAUGA | UCAUAGUUCAGUGAUUCCUCA | Chimp | [1490-1510] ORF |
| 642 | UCCGAAUGGUCAAAGAAAAUU | AAUUUUCUUUGACCAUUCGGA | Chimp | [1210-1230] ORF |
| 643 | UCCCUCAGAUGUCAUGGAAAU | AUUUCCAUGACAUCUGAGGGA | Chimp | [1190-1210] ORF |
| 644 | CAUCCCUCAGAUGUCAUGGAA | UUCCAUGACAUCUGAGGGAUG | Chimp, Rat | [1188-1208] ORF |
| 645 | GCCAGUCACCAUCAUUUCGGU | ACCGAAAUGAUGGUGACUGGC | Chimp | [1160-1180] ORF |
| 646 | UCCCUGAAGGAUUUUCAAAAC | GUUUUGAAAAUCCUUCAGGGA | Chimp | [991-1011] ORF |
| 647 | CAGAACAUUUUCAUGAACCUU | AAGGUUCAUGAAAAUGUUCUG | Chimp | [970-990] ORF |
| 648 | UCAGAACAUUUUCAUGAACCU | AGGUUCAUGAAAAUGUUCUGA | Chimp | [969-989] ORF |
| 649 | ACCGAACCAGCUCUCAGAAUA | UAUUCUGAGAGCUGGUUCGGU | Chimp | [928-948] ORF |
| 650 | GGAGGGCUGCUGAAGUAUCAA | UUGAUACUUCAGCAGCCCUCC | Chimp | [867-887] ORF |
| 651 | UGGCUCUCCAUGAAUGUCCUG | CAGGACAUUCAUGGAGAGCCA | Chimp | [303-323] ORF |
| 652 | CUGGCUCUCCAUGAAUGUCCU | AGGACAUUCAUGGAGAGCCAG | Chimp | [302-322] ORF |
| 653 | GAUUCACUUUUCAGUUCUCAA | UUGAGAACUGAAAAGUGAAUC | Chimp | [2195-2215] 3′UTR |
| 654 | UGAUUCACUUUUCAGUUCUCA | UGAGAACUGAAAAGUGAAUCA | Chimp | [2194-2214] 3′UTR |
| 655 | GAGUCUUUCAGCUGAAAACUU | AAGUUUUCAGCUGAAAGACUC | Chimp | [1962-1982] |
| | | | | ORF, 3′UTR |
| 656 | CAAGACUGUUUAUAGGACGUC | GACGUCCUAUAAACAGUCUUG | | [1789-1809] ORF |
| 657 | CUGUACCUCAGUCAAACAGAU | AUCUGUUUGACUGAGGUACAG | Chimp, Rat, Ms | [1725-1745] ORF |
| 658 | GACUAUGUCAACAUCCAGCUG | CAGCUGGAUGUUGACAUAGUC | Chimp | [1707-1727] ORF |
| 659 | AGAGCCUCAGCAUCUGUUCUU | AAGAACAGAUGCUGAGGCUCU | Chimp | [411-431] ORF |
| 660 | UCAGAUGUCAUGGAAAUCCGA | UCGGAUUUCCAUGACAUCUGA | Chimp | [1194-1214] ORF |
| 661 | UUACAGGUAUAUCCGGAGCAA | UUGCUCCGGAUAUACCUGUAA | Chimp | [1136-1156] ORF |
| 662 | UCAGAAUAUUUCCUUACCAGA | UCUGGUAAGGAAAUAUUCUGA | Chimp | [941-961] ORF |
| 663 | AGUCUUUCAGCUGAAAACUUU | AAAGUUUUCAGCUGAAAGACU | Chimp | [1963-1983] |
| | | | | ORF + 3′UTR |
| 664 | UCCAGCUGUACCUCAGUCAAA | UUUGACUGAGGUACAGCUGGA | Chimp, Rat, Ms | [1720-1740] ORF |
| 665 | CGAAAUUCUGCCCUUCAUUCA | UGAAUGAAGGGCAGAAUUUCG | Chimp | [1418-1438] ORF |
| 666 | AUCCCUCAGAUGUCAUGGAAA | UUUCCAUGACAUCUGAGGGAU | Chimp | [1189-1209] ORF |
| 667 | ACCCAGCACAAAUUUGUGAAG | CUUCACAAAUUUGUGCUGGGU | Chimp | [1023-1043] ORF |
| 668 | CUCAGAAUAUUUCCUUACCAG | CUGGUAAGGAAAUAUUCUGAG | Chimp | [940-960] ORF |
| 669 | AUGUCCUGCUUUUCUGGAAAA | UUUUCCAGAAAAGCAGGACAU | Chimp, Rat | [316-336] ORF |
| 670 | AGAAAACUGUUAGAUGCACAC | GUGUGCAUCUAACAGUUUUCU | Chimp | [2222-2242] 3′UTR |
| 671 | CUUCAUAAACUGAGUAACCAG | CUGGUUACUCAGUUUAUGAAG | Chimp | [1905-1925] ORF |
| 672 | CUGUUGGAUGACUGGAAACCA | UGGUUUCCAGUCAUCCAACAG | Chimp | [1572-1592] ORF |
| 673 | UCAGGAGGGCUGCUGAAGUAU | AUACUUCAGCAGCCCUCCUGA | Chimp | [864-884] ORF |
| 674 | CACAGAAGGUUCCAAGCAGGA | UCCUGCUUGGAACCUUCUGUG | Chimp | [496-516] ORF |
| 675 | UGGAAAUCCGAAUGGUCAAAG | CUUUGACCAUUCGGAUUUCCA | Chimp | [1204-1224] ORF |
| 676 | AGAAUAUUUCCUUACCAGAGU | ACUCUGGUAAGGAAAUAUUCU | Chimp | [943-963] ORF |
| 677 | UCUCAGAAUAUUUCCUUACCA | UGGUAAGGAAAUAUUCUGAGA | Chimp | [939-959] ORF |
| 678 | GAAAAACAGUUGGUGUUUUCU | AGAAAACACCAACUGUUUUUC | | [1855-1875] ORF |
| 679 | GGCUUCCACUCAGUCUUUGAC | GUCAAAGACUGAGUGGAAGCC | | [104-124] 5′UTR |
| 680 | UGAAAACUUUUGCCAUGAAGC | GCUUCAUGGCAAAAGUUUUCA | Chimp | [1974-1994] |
| | | | | ORF + 3′UTR |
| 681 | AGGAAAAACAGUUGGUGUUUU | AAAACACCAACUGUUUUUCCU | | [1853-1873] ORF |
| 682 | UCAUGCACUGAAUUCAAGACU | AGUCUUGAAUUCAGUGCAUGA | Chimp | [1775-1795] ORF |
| 683 | UGGCUUUGGAUUUCUGGACCU | AGGUCCAGAAAUCCAAAGCCA | Chimp | [1089-1109] ORF |
| 684 | GAAUGUCCUGCUUUUCUGGAA | UUCCAGAAAAGCAGGACAUUC | Chimp | [314-334] ORF |
| 685 | CGAGGAUCACAGAAGGUUCCA | UGGAACCUUCUGUGAUCCUCG | Chimp | [489-509] ORF |
| 686 | AAGCUAAUUUUCCACAGACUU | AAGUCUGUGGAAAAUUAGCUU | Chimp | [1069-1089] ORF |
| 687 | UCCGAGGAUCACAGAAGGUUC | GAACCUUCUGUGAUCCUCGGA | Chimp | [487-507] ORF |
| 688 | GGAUCACAGAAGGUUCCAAGC | GCUUGGAACCUUCUGUGAUCC | Chimp | [492-512] ORF |
| 689 | AGGAUCACAGAAGGUUCCAAG | CUUGGAACCUUCUGUGAUCCU | Chimp | [491-511] ORF |

TABLE C
19-mer oligomers for NOX1 (NADPH oxidase 1)

| | | | | | |
|---|---|---|---|---|---|
| | | | | Human-21614529 | Human-7669509 |
| Number | Sense siRNA | AntiSense siRNA | Other Sp | ORF: 207-1901 | ORF: 71-1618 |

| | | | | | |
|---|---|---|---|---|---|
| 1 | CUCUCUGCUUGAAUUUUAA | UUAAAAUUCAAGCAGAGAG | | [376-394] ORF | [240-258] ORF |
| 2 | GGAGUUAUUUUGAAGUCUU | AAGACUUCAAAAUAACUCC | | [799-817] ORF | [663-681] ORF |
| 3 | GGCUGAAUGUUUUCCUGUU | AACAGGAAAACAUUCAGCC | | [265-283] ORF | [129-147] ORF |
| 4 | GCAAAGUGGGUUUUCUAAA | UUUAGAAAACCCACUUUGC | | [1594-1612] ORF | — |
| 5 | GCAGACCACAACCUCAAAA | UUUUGAGGUUGUGGUCUGC | GP | [1476-1494] ORF | [1340-1358] ORF |
| 6 | GGACAGAAAAUCUCAUAAG | CUUAUGAGAUUUUCUGUCC | Rat | [1288-1306] ORF | [1152-1170] ORF |
| 7 | CCGGAGGAGUUAUUUUGAA | UUCAAAAUAACUCCUCCGG | | [794-812] ORF | [658-676] ORF |
| 8 | ACAUGUUUACUGUCACUUU | AAAGUGACAGUAAACAUGU | | [2487-2505] 3′UTR | — |
| 9 | GUAGGGUGACUUUUUAAAU | AUUUAAAAAGUCACCCUAC | | [2371-2389] 3′UTR | — |
| 10 | CCAGGUGAUUUUAUCAAUU | AAUUGAUAAAAUCACCUGG | | [2281-2299] 3′UTR | — |
| 11 | CACUUUAGGAUAAGAAUGU | ACAUUCUUAUCCUAAAGUG | | [2041-2059] 3′UTR | [1701-1719] 3′UTR |
| 12 | GUCACUUUAGGAUAAGAAU | AUUCUUAUCCUAAAGUGAC | | [2039-2057] 3′UTR | [1699-1717] 3′UTR |
| 13 | AGUCACUUUAGGAUAAGAA | UUCUUAUCCUAAAGUGACU | | [2038-2056] 3′UTR | [1698-1716] 3′UTR |
| 14 | AGGUUCAAUUCUACUUCAA | UUGAAGUAGAAUUGAACCU | Rat | [1867-1885] ORF | [1584-1602] ORF |
| 15 | GGCAUCCUUUUACUUUGAC | GUCAAAGUAAAAGGAUGCC | | [1216-1234] ORF | [1080-1098] ORF |
| 16 | GCAGAGUCUUUUGAGAUGU | ACAUCUCAAAAGACUCUGC | | [936-954] ORF | [800-818] ORF |
| 17 | CUCCAGCCUAUCUCAUGAU | AUCAUGAGAUAGGCUGGAG | | [629-647] ORF | [493-511] ORF |
| 18 | CAGUCACUUUAGGAUAAGA | UCUUAUCCUAAAGUGACUG | | [2037-2055] 3′UTR | [1697-1715] 3′UTR |
| 19 | CAAAGUGGGUUUUCUAAAC | GUUUAGAAAACCCACUUUG | | [1595-1613] ORF | — |
| 20 | CGGUCAUUCUUUAUAUCUG | CAGAUAUAAAGAAUGACCG | | [1033-1051] ORF | [897-915] ORF |
| 21 | CGGCAUUGGUGGAAUUGUC | GACAAUUCCACCAAUGCCG | | [869-887] ORF | [733-751] ORF |
| 22 | CCACCUUUUUAUCUUCUAU | AUAGAAGAUAAAAAGGUGG | | [830-848] ORF | [694-712] ORF |
| 23 | GGAGGAGUUAUUUUGAAGU | ACUUCAAAAUAACUCCUCC | | [796-814] ORF | [660-678] ORF |
| 24 | CAGUGGAGUAUGUGACAUU | AAUGUCACAUACUCCACUG | | [697-715] ORF | [561-579] ORF |
| 25 | ACACCUGUUUAACUUUGAC | GUCAAAGUUAAACAGGUGU | | [560-578] ORF | [424-442] ORF |
| 26 | CACACCUGUUUAACUUUGA | UCAAAGUUAAACAGGUGUG | Rabbit | [559-577] ORF | [423-441] ORF |
| 27 | GCUAUUCACAUCAUUGCAC | GUGCAAUGAUGUGAAUAGC | | [543-561] ORF | [407-425] ORF |
| 28 | GGAUCACAACCUCACCUUC | GAAGGUGAGGUUGUGAUCC | Rat, Ms | [488-506] ORF | [352-370] ORF |
| 29 | UCACUUUUGAUAUGGUCUU | AAGACCAUAUCAAAAGUGA | | [2499-2517] 3′UTR | — |
| 30 | AGUAGGGUGACUUUUUAAA | UUUAAAAAGUCACCCUACU | | [2370-2388] 3′UTR | — |
| 31 | CCCAGGUGAUUUUAUCAAU | AUUGAUAAAAUCACCUGGG | | [2280-2298] 3′UTR | — |
| 32 | GCAUUAAACUUUGACAAGG | CCUUGUCAAAGUUUAAUGC | | [1662-1680] ORF | [1379-1397] ORF |
| 33 | AAGUGGGUUUUCUAAACUA | UAGUUUAGAAAACCCACUU | | [1597-1615] ORF | — |
| 34 | CUCCAUUCAUAUCCGAGCA | UGCUCGGAUAUGAAUGGAG | | [1259-1277] ORF | [1123-1141] ORF |
| 35 | AGAUUUCUUCUCCAUUCAU | AUGAAUGGAGAAGAAAUCU | | [1250-1268] ORF | [1114-1132] ORF |
| 36 | UGGCAUCCUUUUACUUUGA | UCAAAGUAAAAGGAUGCCA | | [1215-1233] ORF | [1079-1097] ORF |
| 37 | UCAAUCUCUCUCCUGGAAU | AUUCCAGGAGAGAGAUUGA | | [1197-1215] ORF | [1061-1079] ORF |
| 38 | CAAGUGUGCAGAGUCUUUU | AAAAGACUCUGCACACUUG | | [929-947] ORF | [793-811] ORF |
| 39 | ACAGUGGAGUAUGUGACAU | AUGUCACAUACUCCACUGU | | [696-714] ORF | [560-578] ORF |
| 40 | CCAGCCUAUCUCAUGAUGA | UCAUCAUGAGAUAGGCUGG | | [631-649] ORF | [495-513] ORF |
| 41 | CUAUUCACAUCAUUGCACA | UGUGCAAUGAUGUGAAUAG | | [544-562] ORF | [408-426] ORF |
| 42 | UACAGCUAUUCACAUCAUU | AAUGAUGUGAAUAGCUGUA | | [539-557] ORF | [403-421] ORF |
| 43 | CACUUUUGAUAUGGUCUUA | UAAGACCAUAUCAAAAGUG | | [2500-2518] 3′UTR | — |
| 44 | CCUGAGUUUUGGUUUGUAA | UUACAAACCAAAACUCAGG | | [2315-2333] 3′UTR | — |
| 45 | AGGAUAAGAAUGUGCCUCU | AGAGGCACAUUCUUAUCCU | | [2047-2065] 3′UTR | [1707-1725] 3′UTR |
| 46 | UGAGCAGUCACUUUAGGAU | AUCCUAAAGUGACUGCUCA | | [2033-2051] 3′UTR | [1693-1711] 3′UTR |
| 47 | GGAGUUUUCUUAUGUGGCC | GGCCACAUAAGAAAACUCC | | [1785-1803] ORF | [1502-1520] ORF |
| 48 | CCACAACCUCAAAACAAAA | UUUUGUUUUGAGGUUGUGG | | [1481-1499] ORF | [1345-1363] ORF |
| 49 | ACCACAACCUCAAAACAAA | UUUGUUUUGAGGUUGUGGU | | [1480-1498] ORF | [1344-1362] ORF |
| 50 | AGAAUCCGGAUUGCUGAAU | AUUCAGCAAUCCGGAUUCU | | [147-165] 5′UTR | [11-29] 5′UTR |
| 51 | CCUUUUACUUUGACCUCUG | CAGAGGUCAAAGUAAAAGG | | [1221-1239] ORF | [1085-1103] ORF |
| 52 | UGAUUACCAAGGUUGUUAU | AUAACAACCUUGGUAAUCA | | [1093-1111] ORF | [957-975] ORF |
| 53 | CCGGUCAUUCUUUAUAUCU | AGAUAUAAAGAAUGACCGG | | [1032-1050] ORF | [896-914] ORF |
| 54 | ACCGGUCAUUCUUUAUAUC | GAUAUAAAGAAUGACCGGU | | [1031-1049] ORF | [895-913] ORF |
| 55 | CGGAGGAGUUAUUUUGAAG | CUUCAAAAUAACUCCUCCG | | [795-813] ORF | [659-677] ORF |
| 56 | CAAUAGCCUUGAUUCUCAU | AUGAGAAUCAAGGCUAUUG | | [751-769] ORF | [615-633] ORF |
| 57 | CUGUUUAACUUUGACUGCU | AGCAGUCAAAGUUAAACAG | | [564-582] ORF | [428-446] ORF |
| 58 | CACCUGUUUAACUUUGACU | AGUCAAAGUUAAACAGGUG | | [561-579] ORF | [425-443] ORF |
| 59 | UGAAUUUUAACAGCACGCU | AGCGUGCUGUUAAAAUUCA | | [385-403] ORF | [249-267] ORF |
| 60 | CCGACAAAUACUACUACAC | GUGUAGUAGUAUUUGUCGG | | [313-331] ORF | [177-195] ORF |
| 61 | CCUUCCUGAAAUAUGAGAA | UUCUCAUAUUUCAGGAAGG | | [292-310] ORF | [156-174] ORF |
| 62 | GCCUUCCUGAAAUAUGAGA | UCUCAUAUUUCAGGAAGGC | | [291-309] ORF | [155-173] ORF |
| 63 | CUGUAAAUCUUUCUGGACA | UGUCCAGAAAGAUUUACAG | | [2201-2219] 3′UTR | — |
| 64 | UGCAUUCAACUUCGUUACU | AGUAACGAAGUUGAAUGCA | | [2099-2117] 3′UTR | — |
| 65 | GCAUUUUGUCUCUUUGUAU | AUACAAAGAGACAAAAUGC | | [1984-2002] 3′UTR | [1644-1662] 3′UTR |
| 66 | GUAGUGGGAGUUUUCUUAU | AUAAGAAAACUCCCACUAC | | [1779-1797] ORF | [1496-1514] ORF |
| 67 | CCAAGUCUGUAGUGGGAGU | ACUCCCACUACAGACUUGG | | [1771-1789] ORF | [1488-1506] ORF |
| 68 | CCGGAUGGGACAGCAAUAU | AUAUUGCUGUCCCAUCCGG | | [1630-1648] ORF | — |
| 69 | AAAGUGGGUUUUCUAAACU | AGUUUAGAAAACCCACUUU | | [1596-1614] ORF | — |
| 70 | ACAAAUUCCAGUGUGCAGA | UCUGCACACUGGAAUUUGU | | [1462-1480] ORF | [1326-1344] ORF |
| 71 | GGUACAAAUUCCAGUGUGC | GCACACUGGAAUUUGUACC | | [1459-1477] ORF | [1323-1341] ORF |
| 72 | CUUCUAUCUUGAAAUCCAU | AUGGAUUUCAAGAUAGAAG | | [1438-1456] ORF | [1302-1320] ORF |
| 73 | GCAUCCUUUUACUUUGACC | GGUCAAAGUAAAAGGAUGC | | [1217-1235] ORF | [1081-1099] ORF |
| 74 | CAGAGUCUUUUGAGAUGUG | CACAUCUCAAAAGACUCUG | | [937-955] ORF | [801-819] ORF |
| 75 | GAGUUAUUUUGAAGUCUUC | GAAGACUUCAAAAUAACUC | | [800-818] ORF | [664-682] ORF |
| 76 | GAGGAGUUAUUUUGAAGUC | GACUUCAAAAUAACUCCUC | | [797-815] ORF | [661-679] ORF |
| 77 | CGACAGUGGAGUAUGUGAC | GUCACAUACUCCACUGUCG | | [694-712] ORF | [558-576] ORF |
| 78 | UCCAGCCUAUCUCAUGAUG | CAUCAUGAGAUAGGCUGGA | | [630-648] ORF | [494-512] ORF |
| 79 | ACCUGUUUAACUUUGACUG | CAGUCAAAGUUAAACAGGU | | [562-580] ORF | [426-444] ORF |
| 80 | UGCACACCUGUUUAACUUU | AAAGUUAAACAGGUGUGCA | | [557-575] ORF | [421-439] ORF |
| 81 | ACAUCAUUGCACACCUGUU | AACAGGUGUGCAAUGAUGU | | [550-568] ORF | [414-432] ORF |
| 82 | GCCUAUAUGAUCUGCCUAC | GUAGGCAGAUCAUAUAGGC | | [519-537] ORF | [383-401] ORF |
| 83 | GGCCGACAAAUACUACUAC | GUAGUAGUAUUUGUCGGCC | | [311-329] ORF | [175-193] ORF |
| 84 | UGAAUGUUUUCCUGUUUGU | ACAAACAGGAAAACAUUCA | | [268-286] ORF | [132-150] ORF |
| 85 | CUGAAUGUUUUCCUGUUUG | CAAACAGGAAAACAUUCAG | | [267-285] ORF | [131-149] ORF |
| 86 | CAGUUUUGUUUCUGGUUGU | ACAACCAGAAACAAAACUG | | [238-256] ORF | [102-120] ORF |
| 87 | UGUUUACUGUCACUUUUGA | UCAAAAGUGACAGUAAACA | | [2490-2508] 3′UTR | — |
| 88 | CAACUUAAGAACUUCUGAA | UUCAGAAGUUCUUAAGUUG | | [2128-2146] 3′UTR | — |
| 89 | CAACUUCGUUACUUGAGCU | AGCUCAAGUAACGAAGUUG | | [2105-2123] 3′UTR | — |
| 90 | UGAUUGCAUUCAACUUCGU | ACGAAGUUGAAUGCAAUCA | | [2095-2113] 3′UTR | — |
| 91 | UCCCUGGUAUUCUUUUUUU | AAAAAAAGAAUACCAGGGA | | [2077-2095] 3′UTR | — |
| 92 | UGCAUUUUGUCUCUUUGUA | UACAAAGAGACAAAAUGCA | | [1983-2001] 3′UTR | [1643-1661] 3′UTR |
| 93 | GUGGGAGUUUUCUUAUGUG | CACAUAAGAAAACUCCCAC | | [1782-1800] ORF | [1499-1517] ORF |
| 94 | AGUCUGUAGUGGGAGUUUU | AAAACUCCCACUACAGACU | | [1774-1792] ORF | [1491-1509] ORF |
| 95 | CAAGUCUGUAGUGGGAGUU | AACUCCCACUACAGACUUG | | [1772-1790] ORF | [1489-1507] ORF |
| 96 | GGACAAUGAGUUUUCUACA | UGUAGAAAACUCAUUGUCC | | [1736-1754] ORF | [1453-1471] ORF |
| 97 | CAGCAAUAUUGUUGGUCAU | AUGACCAACAAUAUUGCUG | | [1640-1658] ORF | — |
| 98 | CACAACCUGAAAACAAAAA | UUUUUGUUUUGAGGUUGUG | | [1482-1500] ORF | [1346-1364] ORF |
| 99 | GACCACAACCUCAAAACAA | UUGUUUUGAGGUUGUGGUC | | [1479-1497] ORF | [1343-1361] ORF |
| 100 | CUAUCUUGAAAUCCAUCUG | CAGAUGGAUUUCAAGAUAG | | [1441-1459] ORF | [1305-1323] ORF |
| 101 | CCCUUUGCUUCUAUCUUGA | UCAAGAUAGAAGCAAAGGG | | [1431-1449] ORF | [1295-1313] ORF |
| 102 | UCCUUUUACUUUGACCUCU | AGAGGUCAAAGUAAAAGGA | | [1220-1238] ORF | [1084-1102] ORF |
| 103 | GUCAUUCUUUAUAUCUGUG | CACAGAUAUAAAGAAUGAC | | [1035-1053] ORF | [899-917] ORF |
| 104 | AGAGCAUGAAUGAGAGUCA | UGACUCUCAUUCAUGCUCU | | [904-922] ORF | [768-786] ORF |
| 105 | GCUUAGGGAUUCACGGCAU | AUGCCGUGAAUCCCUAAGC | | [856-874] ORF | [720-738] ORF |
| 106 | ACCACCUUUUUAUCUUCUA | UAGAAGAUAAAAAGGUGGU | | [829-847] ORF | [693-711] ORF |
| 107 | AGGAGUUAUUUUGAAGUCU | AGACUUCAAAAUAACUCCU | | [798-816] ORF | [662-680] ORF |
| 108 | UGAUUCUCAUGGUAACUUC | GAAGUUACCAUGAGAAUCA | GP, Rabbit | [760-778] ORF | [624-642] ORF |
| 109 | CUGGAGUGAUCAUGACAAU | AUUGUCAUGAUCACUCCAG | | [736-754] ORF | [600-618] ORF |
| 110 | GGGUAGGUGUGUGUUUUUC | GAAAAACACACACCUACCC | | [67-85] 5′UTR | — |
| 111 | CCUACAUACAGCUAUUCAC | GUGAAUAGCUGUAUGUAGG | | [533-551] ORF | [397-415] ORF |
| 112 | UGAAAUAUGAGAAGGCCGA | UCGGCCUUCUCAUAUUUCA | | [298-316] ORF | [162-180] ORF |
| 113 | GAAGGCGGAAUAAACUUAU | AUAAGUUUAUUCCGCCUUC | | [29-47] 5′UTR | — |
| 114 | AGUUUUGUUUCUGGUUGUU | AACAACCAGAAACAAAACU | | [239-257] ORF | [103-121] ORF |
| 115 | CUGUCACUUUUGAUAUGGU | ACCAUAUCAAAAGUGACAG | | [2496-2514] 3′UTR | — |
| 116 | CCCAUAUAACACCAACAGU | ACUGUUGGUGUUAUAUGGG | | [2466-2484] 3′UTR | — |
| 117 | ACAACUAAUCCCAGGUGAU | AUCACCUGGGAUUAGUUGU | | [2271-2289] 3′UTR | — |
| 118 | CAGAGUUAUACAACUAAUC | GAUUAGUUGUAUAACUCUG | | [2262-2280] 3′UTR | — |
| 119 | GUUCAGAGUUAUACAACUA | UAGUUGUAUAACUCUGAAC | | [2259-2277] 3′UTR | — |
| 120 | GGUUCAGAGUUAUACAACU | AGUUGUAUAACUCUGAACC | | [2258-2276] 3′UTR | — |
| 121 | GUGGUUCAGAGUUAUACAA | UUGUAUAACUCUGAACCAC | | [2256-2274] 3′UTR | — |
| 122 | UGGGAAACUGGGUGGUUAA | UUAACCACCCAGUUUCCCA | | [208-226] ORF | [72-90] ORF |
| 123 | GUAAUUUACUUGGUCUCGU | ACGAGACCAAGUAAAUUAC | | [2008-2026] 3′UTR | [1668-1686] 3′UTR |
| 124 | UCAGUAAUUUACUUGGUCU | AGACCAAGUAAAUUACUGA | | [2005-2023] 3′UTR | [1665-1683] 3′UTR |
| 125 | AGGACGGUAAUCUGCAUUU | AAAUGCAGAUUACCGUCCU | | [1971-1989] 3′UTR | [1631-1649] 3′UTR |
| 126 | AGCAUUAAACUUUGACAAG | CUUGUCAAAGUUUAAUGCU | | [1661-1679] ORF | [1378-1396] ORF |
| 127 | GCCUUUUCCUGGUUCAACA | UGUUGAACCAGGAAAAGGC | | [1536-1554] ORF | — |
| 128 | CCUCAAAACAAAAAAGAUC | GAUCUUUUUUGUUUUGAGG | | [1487-1505] ORF | [1351-1365] (16/16) |
| | | | | | ORF |
| 129 | CAGACCACAACCUCAAAAC | GUUUUGAGGUUGUGGUCUG | | [1477-1495] ORF | [1341-1359] ORF |
| 130 | AGCAGAAGGUUGUGAUUAC | GUAAUCACAACCUUCUGCU | | [1081-1099] ORF | [945-963] ORF |
| 131 | UGCAGAGUCUUUUGAGAUG | CAUCUCAAAAGACUCUGCA | | [935-953] ORF | [799-817] ORF |
| 132 | GGAGAGCAUGAAUGAGAGU | ACUCUCAUUCAUGCUCUCC | | [902-920] ORF | [766-784] ORF |
| 133 | ACAUCUUAAAGGCUCACAG | CUGUGAGCCUUUAAGAUGU | | [86-104] 5′UTR | — |
| 134 | CUAUAUGAUCUGCCUACAU | AUGUAGGCAGAUCAUAUAG | | [521-539] ORF | [385-403] ORF |
| 135 | GAAAAAUCCUUGGGUCAAC | GUUGACCCAAGGAUUUUUC | | [334-352] ORF | [198-216] ORF |
| 136 | AGGCCGACAAAUACUACUA | UAGUAGUAUUUGUCGGCCU | | [310-328] ORF | [174-192] ORF |
| 137 | GGGCUGAAUGUUUUCCUGU | ACAGGAAAACAUUCAGCCC | | [264-282] ORF | [128-146] ORF |
| 138 | UCCCAGGUGAUUUUAUCAA | UUGAUAAAAUCACCUGGGA | | [2279-2297] 3′UTR | — |
| 139 | UGUAAAUCUUUCUGGACAG | CUGUCCAGAAAGAUUUACA | | [2202-2220] 3′UTR | — |
| 140 | CCCUGGUAUUCUUUUUUUG | CAAAAAAAGAAUACCAGGG | | [2078-2096] 3′UTR | — |
| 141 | CUCCCUGGUAUUCUUUUUU | AAAAAAGAAUACCAGGGAG | | [2076-2094] 3′UTR | — |
| 142 | GCCUUGACUCCCUGGUAUU | AAUACCAGGGAGUCAAGGC | | [2069-2087] 3′UTR | — |
| 143 | CAGUAAUUUACUUGGUCUC | GAGACCAAGUAAAUUACUG | | [2006-2024] 3′UTR | [1666-1684] 3′UTR |
| 144 | CUCUUUGUAUCUUCAGUAA | UUACUGAAGAUACAAAGAG | | [1993-2011] 3′UTR | [1653-1671] 3′UTR |
| 145 | CACCUCUUGACAAUGGGAA | UUCCGAUUGUCAAGAGGUG | | [195-213] | [59-77] |
| | | | | 5′UTR + ORF | 5′UTR + ORF |
| 146 | AAGGUUCAAUUCUACUUCA | UGAAGUAGAAUUGAACCUU | Rat | [1866-1884] ORF | [1583-1601] ORF |
| 147 | AGUGGGAGUUUUCUUAUGU | ACAUAAGAAAACUCCCACU | | [1781-1799] ORF | [1498-1516] ORF |
| 148 | UGUAGUGGGAGUUUUCUUA | UAAGAAAACUCCCACUACA | | [1778-1796] ORF | [1495-1513] ORF |
| 149 | CUGUAGUGGGAGUUUUCUU | AAGAAAACUCCCACUACAG | | [1777-1795] ORF | [1494-1512] ORF |
| 150 | UGGUCAUGCAGCAUUAAAC | GUUUAAUGCUGCAUGACCA | GP, Rat, | [1652-1670] ORF | [1369-1387] ORF |
| | | | Ms | | |
| 151 | GGUUUUCUAAACUACCGUC | GACGGUAGUUUAGAAAACC | | [1602-1620] ORF | — |
| 152 | UGGAGGAAUUAGGCAAAGU | ACUUUGCCUAAUUCCUCCA | | [1582-1600] ORF | — |
| 153 | GGAUUGCUGAAUCUUCCCU | AGGGAAGAUUCAGCAAUCC | | [154-172] 5′UTR | [18-36] 5′UTR |
| 154 | CUUUGCUUCUAUCUUGAAA | UUUCAAGAUAGAAGCAAAG | | [1433-1451] ORF | [1297-1315] ORF |
| 155 | CCUUUGCUUCUAUCUUGAA | UUCAAGAUAGAAGCAAAGG | | [1432-1450] ORF | [1296-1314] ORF |
| 156 | UGCACCGGUCAUUCUUUAU | AUAAAGAAUGACCGGUGCA | | [1028-1046] ORF | [892-910] ORF |
| 157 | GUGCAGAGUCUUUUGAGAU | AUCUCAAAAGACUCUGCAC | | [934-952] ORF | [798-816] ORF |
| 158 | AGGAGAGCAUGAAUGAGAG | CUCUCAUUCAUGCUCUCCU | | [901-919] ORF | [765-783] ORF |
| 159 | CUCACCACCUUUUUAUCUU | AAGAUAAAAAGGUGGUGAG | | [826-844] ORF | [690-708] ORF |
| 160 | GUAUACUCACCACCUUUUU | AAAAAGGUGGUGAGUAUAC | | [821-839] ORF | [685-703] ORF |
| 161 | CACAUCUUAAAGGCUCACA | UGUGAGCCUUUAAGAUGUG | | [85-103] 5′UTR | — |
| 162 | UCACAUCUUAAAGGCUCAC | GUGAGCCUUUAAGAUGUGA | | [84-102] 5′UTR | — |
| 163 | GUGGAGUAUGUGACAUUCA | UGAAUGUCACAUACUCCAC | | [699-717] ORF | [563-581] ORF |
| 164 | CCAUUCUCUCCAGCCUAUC | GAUAGGCUGGAGAGAAUGG | | [622-640] ORF | [486-504] ORF |
| 165 | CCUAUAUGAUCUGCCUACA | UGUAGGCAGAUCAUAUAGG | | [520-538] ORF | [384-402] ORF |
| 166 | UCCUGUGUGUCGCAAUCUG | CAGAUUGCGACACACAGGA | | [413-431] ORF | [277-295] ORF |
| 167 | AGAAAAAUCCUUGGGUCAA | UUGACCCAAGGAUUUUUCU | | [333-351] ORF | [197-215] ORF |
| 168 | AGGGCUGAAUGUUUUCCUG | CAGGAAAACAUUCAGCCCU | | [263-281] ORF | [127-145] ORF |
| 169 | AAACAAAAUUACCGAGAAC | GUUCUCGGUAAUUUUGUUU | | [2441-2459] 3′UTR | — |
| 170 | UGGUUUGUAAUCUUUUGUC | GACAAAAGAUUACAAACCA | | [2324-2342] 3′UTR | — |
| 171 | GAGUUUUGGUUUGUAAUCU | AGAUUACAAACCAAAACUC | | [2318-2336] 3′UTR | — |
| 172 | CAGGUGAUUUUAUCAAUUC | GAAUUGAUAAAAUCACCUG | | [2282-2300] 3′UTR | — |
| 173 | UGAAGUUCUUAAAGCCCAU | AUGGGCUUUAAGAACUUCA | | [2160-2178] 3′UTR | — |
| 174 | GGGAAACUGGGUGGUUAAC | GUUAACCACCCAGUUUCCC | | [209-227] ORF | [73-91] ORF |
| 175 | GCAACUUAAGAACUUCUGA | UCAGAAGUUCUUAAGUUGC | | [2127-2145] 3′UTR | — |
| 176 | AACUUCGUUACUUGAGCUU | AAGCUCAAGUAACGAAGUU | | [2106-2124] 3′UTR | — |
| 177 | CUCUUGACAAUGGGAAACU | AGUUUCCCAUUGUCAAGAG | | [198-216] | [62-80] |
| | | | | 5′UTR + ORF | 5′UTR + ORF |
| 178 | ACGGUAAUCUGCAUUUUGU | ACAAAAUGCAGAUUACCGU | | [1974-1992] 3′UTR | [1634-1652] 3′UTR |
| 179 | CUCGGACUUUGGCAAAGAG | CUCUUUGCCAAAGUCCGAG | | [1804-1822] ORF | [1521-1539] ORF |
| 180 | CAAUAUUGUUGGUCAUGCA | UGCAUGACCAACAAUAUUG | | [1643-1661] ORF | [1367-1378] (12/12) |
| | | | | | ORF |
| 181 | GCAAUAUUGUUGGUCAUGC | GCAUGACCAACAAUAUUGC | | [1642-1660] ORF | [1367-1377] (11/11) |
| | | | | | ORF |
| 182 | ACAGCAAUAUUGUUGGUCA | UGACCAACAAUAUUGCUGU | | [1639-1657] ORF | — |
| 183 | GAAUUAGGCAAAGUGGGUU | AACCCACUUUGCCUAAUUC | | [1587-1605] ORF | — |
| 184 | AGAUGGAGGAAUUAGGCAA | UUGCCUAAUUCCUCCAUCU | Ms | [1579-1597] ORF | — |
| 185 | ACAGGAGAUGGAGGAAUUA | UAAUUCCUCCAUCUCCUGU | | [1574-1592] ORF | — |
| 186 | CAACCUCAAAACAAAAAAG | CUUUUUUGUUUUGAGGUUG | | [1464-1502] ORF | [1348-1366] ORF |
| 187 | CCAGGAUUGAAGUGGAUGG | CCAUCCACUUCAAUCCUGG | | [1336-1354] ORF | [1200-1218] ORF |
| 188 | GACAGAAAAUCUCAUAAGG | CCUUAUGAGAUUUUCUGUC | Rat | [1289-1307] ORF | [1153-1171] ORF |
| 189 | GAAUGGCAUCCUUUUACUU | AAGUAAAAGGAUGCCAUUC | | [1212-1230] ORF | [1076-1094] ORF |
| 190 | GUUUUGGAAUUGCAGAUGA | UCAUCUGCAAUUCCAAAAC | Rat, Ms | [1125-1143] ORF | [989-1007] ORF |
| 191 | AAGUUUUGGAAUUGCAGAU | AUCUGCAAUUCCAAAACUU | Rat | [1123-1141] ORF | [967-1005] ORF |
| 192 | GUCAAACAGAGGAGAGCAU | AUGCUCUCCUCUGUUUGAC | | [892-910] ORF | [756-774] ORF |
| 193 | CUUAGGGAUUCACGGCAUU | AAUGCGGUGAAUCCCUAAG | | [857-875] ORF | [721-739] ORF |
| 194 | GAAGUCUUCUGGUAUACUC | GAGUAUACCAGAAGACUUC | | [810-828] ORF | [674-692] ORF |
| 195 | UUUGAAGUCUUCUGGUAUA | UAUACCAGAAGACUUCAAA | | [807-825] ORF | [671-689] ORF |
| 196 | UUGAUUCUCAUGGUAACUU | AAGUUACCAUGAGAAUCAA | GP, Rabbit | [759-777] ORF | [623-641] ORF |
| 197 | UCCCUUGCCUCCAUUCUCU | AGAGAAUGGAGGCAAGGGA | | [612-630] ORF | [476-494] ORF |
| 198 | GAGAAAGCAAUUGGAUCAC | GUGAUCCAAUUGCUUUCUC | | [476-494] ORF | [340-358] ORF |
| 199 | AAGGCGGAAUAAACUUAUU | AAUAAGUUUAUUCCGCCUU | | [30-48] 5′UTR | — |
| 200 | CUGAGUUUUGGUUUGUAAU | AUUACAAACCAAAACUCAG | | [2316-2334] 3′UTR | — |
| 201 | GGUGAUUUUAUCAAUUCCA | UGGAAUUGAUAAAAUCACC | | [2264-2302] 3′UTR | — |
| 202 | CUGAAGUUCUUAAAGUUCU | AGAACUUUAAGAACUUCAG | | [2142-2160] 3′UTR | — |
| 203 | AUCUGCAUUUUGUCUCUUU | AAAGAGACAAAAUGCAGAU | | [1960-1996] 3′UTR | [1640-1658] 3′UTR |
| 204 | GAAACAGAAAACCUCCUUU | AAAGGAGGUUUUCUGUUUC | Rat | [1703-1721] ORF | [1420-1438] ORF |
| 205 | UGUUGGUCAUGCAGCAUUA | UAAUGCUGCAUGACCAACA | | [1849-1867] ORF | [1367-1384] (18/18) |
| | | | | | ORF |
| 206 | GCUGAAUCUUCCCUGUUGC | GCAACAGGGAAGAUUCAGC | | [159-177] 5′UTR | [23-41] 5′UTR |
| 207 | ACAACCUGUUGACUUCCCU | AGGGAAGUCAACAGGUUGU | | [1552-1570] ORF | — |
| 208 | CCUUUUCCUGGUUCAACAA | UUGUUGAACCAGGAAAAGG | | [1537-1555] ORF | — |
| 209 | GGUGCCUUUUCCUGGUUCA | UGAACCAGGAAAAGGCACC | GP | [1533-1551] ORF | — |
| 210 | UGCAGACCACAACCUCAAA | UUUGAGGUUGUGGUCUGCA | GP | [1475-1493] ORF | [1339-1357] ORF |
| 211 | GGCUUUCGAACAACAAUAU | AUAUUGUUGUUCGAAAGCC | | [1307-1325] ORF | [1171-1189] ORF |
| 212 | GGGACUGGACAGAAAAUCU | AGAUUUUCUGUCCAGUCCC | Rat | [1282-1300] ORF | [1146-1164] ORF |
| 213 | GGACAAAUGUUCCAUUCCU | AGGAAUGGAACAUUUGUCC | | [115-133] 5′UTR | — |
| 214 | CACCACCUUUUUAUCUUCU | AGAAGAUAAAAAGGUGGUG | | [828-846] ORF | [692-710] ORF |
| 215 | UCACCACCUUUUUAUCUUC | GAAGAUAAAAAGGUGGUGA | | [827-845] ORF | [691-709] ORF |
| 216 | AUACUCACCACCUUUUUAU | AUAAAAAGGUGGUGAGUAU | | [823-841] ORF | [687-705] ORF |
| 217 | CUCCAUUCUCUCCAGCCUA | UAGGCUGGAGAGAAUGGAG | | [620-638] ORF | [484-502] ORF |
| 218 | CACUGAGAAAGCAAUUGGA | UCCAAUUGCUUUCUCAGUG | | [472-490] ORF | [336-354] ORF |
| 219 | UGGUUAGGGCUGAAUGUUU | AAACAUUCAGCCCUAACCA | | [258-276] ORF | [122-140] ORF |
| 220 | GGUUUGUAAUCUUUUGUCC | GGACAAAAGAUUACAAACC | | [2325-2343] 3′UTR | — |
| 221 | UGAGUUUUGGUUUGUAAUC | GAUUACAAACCAAAACUCA | | [2317-2335] 3′UTR | — |
| 222 | UCAAUUCCAGUGUUACCAU | AUGGUAACACUGGAAUUGA | | [2294-2312] 3′UTR | [1329-1339] (11/11) |
| | | | | | ORF |
| 223 | CUUCUGAAGUUCUUAAAGU | ACUUUAAGAACUUCAGAAG | | [2139-2157] 3′UTR | — |
| 224 | AGCAACUUAAGAACUUCUG | CAGAAGUUCUUAAGUUGCU | | [2126-2144] 3′UTR | — |
| 225 | CUUCGUUACUUGAGCUUCA | UGAAGCUCAAGUAACGAAG | | [2108-2126] 3′UTR | — |
| 226 | CUUCAGUAAUUUACUUGGU | ACCAAGUAAAUUACUGAAG | | [2003-2021] 3′UTR | [1663-1681] 3′UTR |
| 227 | CAGUAAUUGAGUUAUAGGA | UCCUAUAACUCAAUUACUG | | [1949-1967] 3′UTR | [1615-1627] (13/13) |
| | | | | | ORF + 3′UTR |
| 228 | CCUCUUGACAAUGGGAAAC | GUUUCCCAUUGUCAAGAGG | | [197-215] | [61-79] |
| | | | | 5′UTR + ORF | 5′UTR + ORF |
| 229 | CGGUAAUCUGCAUUUUGUC | GACAAAAUGCAGAUUACCG | | [1975-1993] 3′UTR | [1635-1653] 3′UTR |
| 230 | AUAGGAAUAAGGACGGUAA | UUACCGUCCUUAUUCCUAU | | [1962-1980] 3′UTR | [1622-1640] 3′UTR |
| 231 | UGAGUUAUAGGAAUAAGGA | UCCUUAUUCCUAUAACUCA | | [1956-1974] 3′UTR | [1616-1634] |
| | | | | | ORF + 3′UTR |
| 232 | GGAAUUAGGCAAAGUGGGU | ACCCACUUUGCCUAAUUCC | | [1586-1604] ORF | — |
| 233 | CAGGAUUGAAGUGGAUGGU | ACCAUCCACUUCAAUCCUG | | [1337-1355] ORF | [1201-1219] ORF |
| 234 | GGGCUUUCGAACAACAAUA | UAUUGUUGUUCGAAAGCCC | | [1306-1324] ORF | [1170-1188] ORF |
| 235 | CAGAUGAACAAGCGUGGCU | AGCCACGCUUGUUCAUCUG | | [1137-1155] ORF | [1001-1019] ORF |
| 236 | ACAGAGGAGAGCAUGAAUG | CAUUCAUGCUCUCCUCUGU | | [897-915] ORF | [761-779] ORF |
| 237 | UCUUCUAUAUCCUUGGCUU | AAGCCAAGGAUAUAGAAGA | | [841-859] ORF | [705-723] ORF |
| 238 | UGGAGUGAUCAUGACAAUA | UAUUGUCAUGAUCACUCCA | | [737-755] ORF | [601-619] ORF |
| 239 | ACUGGAGUGAUCAUGACAA | UUGUCAUGAUCACUCCAGU | | [735-753] ORF | [599-617] ORF |
| 240 | CACACUGAGAAAGCAAUUG | CAAUUGCUUUCUCAGUGUG | | [470-488] ORF | [334-352] ORF |
| 241 | GCUUCCUGUGUGUCGCAAU | AUUGCGACACACAGGAAGC | | [410-428] ORF | [274-292] ORF |
| 242 | UGUUUGGUUAGGGCUGAAU | AUUCAGCCCUAACCAAACA | | [254-272] ORF | [116-136] ORF |
| 243 | GUGUUACCAUCUCCUGAGU | ACUCAGGAGAUGGUAACAC | | [2303-2321] 3′UTR | — |
| 244 | CAAUUCCAGUGUUACCAUC | GAUGGUAACACUGGAAUUG | | [2295-2313] 3′UTR | [1329-1339] (11/11) |
| | | | | | ORF |
| 245 | GUGAUUUUAUCAAUUCCAG | CUGGAAUUGAUAAAAUCAC | | [2285-2303] 3′UTR | — |
| 246 | CAGCAACUUAAGAACUUCU | AGAAGUUCUUAAGUUGCUG | | [2125-2143] 3′UTR | — |
| 247 | ACUCCCUGGUAUUCUUUUU | AAAAAGAAUACCAGGGAGU | | [2075-2093] 3′UTR | — |
| 248 | AGGUUUGAGCAGUCACUUU | AAAGUGACUGCUCAAACCU | | [2028-2048] 3′UTR | [1688-1708] 3′UTR |
| 249 | CUGCAUUUUGUCUCUUUGU | ACAAAGAGACAAAAUGCAG | | [1962-2000] 3′UTR | [1642-1660] 3′UTR |
| 250 | GUAAUCUGCAUUUUGUCUC | GAGACAAAAUGCAGAUUAC | | [1977-1995] 3′UTR | [1637-1655] 3′UTR |
| 251 | ACCUCUUGACAAUGGGAAA | UUUCCCAUUGUCAAGAGGU | | [196-214] | [60-78] |
| | | | | 5′UTR + ORF | 5′UTR + ORF |
| 252 | UUGUUGGUCAUGCAGCAUU | AAUGCUGCAUGACCAACAA | | [1648-1666] ORF | [1367-1383] (17/17) |
| | | | | | ORF |
| 253 | GGCAAAGUGGGUUUUCUAA | UUAGAAAACCCACUUUGCC | | [1593-1611] ORF | — |
| 254 | GUGCCUUUUCCUGGUUCAA | UUGAACCAGGAAAAGGCAC | GP | [1534-1552] ORF | — |
| 255 | CCUGAGUCUUGGAAGUGGA | UCCACUUCCAAGACUCAGG | GP, Rat, Ms | [1005-1023] ORF | [869-867] ORF |
| 256 | CUACUGAGUUCAUCCGGAG | CUCCGGAUGAACUCAGUAG | | [781-799] ORF | [645-663] ORF |
| 257 | GCUACUGAGUUCAUCCGGA | UCCGGAUGAACUCAGUAGC | | [780-798] ORF | [644-662] ORF |
| 258 | CCUAUCUCAUGAUGAGAAA | UUUCUCAUCAUGAGAUAGG | | [635-653] ORF | [499-517] ORF |
| 259 | UGGUGGCCUAUAUGAUCUG | CAGAUCAUAUAGGCCACCA | | [514-532] ORF | [378-396] ORF |
| 260 | CUGGUGGCCUAUAUGAUCU | AGAUCAUAUAGGCCACCAG | | [513-531] ORF | [377-395] ORF |
| 261 | ACAACCUCACCUUCCACAA | UUGUGGAAGGUGAGGUUGU | | [493-511] ORF | [357-375] ORF |
| 262 | UUGUUUGGUUAGGGCUGAA | UUCAGCCCUAACCAAACAA | | [253-271] ORF | [117-135] ORF |
| 263 | AGAAGGAAGGCGGAAUAAA | UUUAUUCCGCCUUCCUUCU | | [24-42] 5′UTR | — |
| 264 | UGGUGGUUCAGAGUUAUAC | GUAUAACUCUGAACCACCA | | [2254-2272] 3′UTR | — |
| 265 | GGUUUGGUGGUUCAGAGUU | AACUCUGAACCACCAAACC | | [2250-2288] 3′UTR | — |
| 266 | GAGGUUUGGUGGUUCAGAG | CUCUGAACCACCAAACCUC | | [2248-2266] 3′UTR | — |
| 267 | CAGAGAAGGAAGGCGGAAU | AUUCCGCCUUCCUUCUCUG | | [21-39] 5′UTR | — |
| 268 | AGCCCAUGGAUCCUUUCUC | GAGAAAGGAUCCAUGGGCU | | [2172-2190] 3′UTR | — |
| 269 | GAAGUUCUUAAAGCCCAUG | CAUGGGCUUUAAGAACUUC | | [2161-2179] 3′UTR | — |
| 270 | UCAGCAACUUAAGAACUUC | GAAGUUCUUAAGUUGCUGA | | [2124-2142] 3′UTR | — |
| 271 | UGACUCCCUGGUAUUCUUU | AAAGAAUACCAGGGAGUCA | | [2073-2091] 3′UTR | — |
| 272 | UUGACUCCCUGGUAUUCUU | AAGAAUACCAGGGAGUCAA | | [2072-2090] 3′UTR | — |
| 273 | UCAGGUUUGAGCAGUCACU | AGUGACUGCUCAAACCUGA | | [2026-2044] 3′UTR | [1686-1704] 3′UTR |
| 274 | CGUCAGGUUUGAGCAGUCA | UGACUGCUCAAACCUGACG | | [2024-2042] 3′UTR | [1684-1702] 3′UTR |
| 275 | UCGUCAGGUUUGAGCAGUC | GACUGCUCAAACCUGACGA | | [2023-2041] 3′UTR | [1683-1701] 3′UTR |
| 276 | CUCGUCAGGUUUGAGCAGU | ACUGCUCAAACCUGACGAG | | [2022-2040] 3′UTR | [1662-1700] 3′UTR |
| 277 | UACUUGGUCUCGUCAGGUU | AACCUGACGAGACCAAGUA | | [2014-2032] 3′UTR | [1674-1692] 3′UTR |
| 278 | UCUCUUUGUAUCUUCAGUA | UACUGAAGAUACAAAGAGA | | [1992-2010] 3′UTR | [1652-1670] 3′UTR |
| 279 | UCUGCAUUUUGUCUCUUUG | CAAAGAGACAAAAUGCAGA | | [1981-1999] 3′UTR | [1641-1659] 3′UTR |
| 280 | GACGGUAAUCUGCAUUUUG | CAAAAUGCAGAUUACCGUC | | [1973-1991] 3′UTR | [1633-1651] 3′UTR |
| 281 | GGGACAGCAAUAUUGUUGG | CCAACAAUAUUGCUGUCCC | | [1636-1654] ORF | — |
| 282 | GAAAAUCUCAUAAGGGCUU | AAGCCCUUAUGAGAUUUUC | | [1293-1311] ORF | [1157-1175] ORF |
| 283 | UUGUUAUGCACCCAUCCAA | UUGGAUGGGUGCAUAACAA | | [1105-1123] ORF | [989-987] ORF |
| 284 | CCAGCAGAAGGUUGUGAUU | AAUCACAACCUUCUGCUGG | | [1079-1097] ORF | [943-951] ORF |
| 285 | CUGAGUCUUGGAAGUGGAU | AUCCACUUCCAAGACUCAG | GP, Rat, Ms | [1006-1024] ORF | [870-888] ORF |
| 286 | CUGCUCUCUGCUUGAAUUU | AAAUUCAAGCAGAGAGCAG | | [373-391] ORF | [237-255] ORF |
| 287 | GCGGAAUAAACUUAUUCAU | AUGAAUAAGUUUAUUCCGC | | [33-51] 5′UTR | — |
| 288 | AGCAGAGGUUUGGUGGUUC | GAACCACCAAACCUCUGCU | | [2244-2262] 3′UTR | — |
| 289 | UGAUAGCAGAGGUUUGGUG | CACCAAACCUCUGCUAUCA | | [2240-2258] 3′UTR | — |
| 290 | CCCAUGGAUCCUUUCUCAG | CUGAGAAAGGAUCCAUGGG | | [2174-2192] 3′UTR | — |
| 291 | CUGAAGUUCUUAAAGCCCA | UGGGCUUUAAGAACUUCAG | | [2159-2177] 3′UTR | — |
| 292 | UUCAGCAACUUAAGAACUU | AAGUUCUUAAGUUGCUGAA | | [2123-2141] 3′UTR | — |
| 293 | GACUCCCUGGUAUUCUUUU | AAAAGAAUACCAGGGAGUC | | [2074-2092] 3′UTR | — |
| 294 | CAGGUUUGAGCAGUCACUU | AAGUGACUGCUCAAACCUG | | [2027-2045] 3′UTR | [1687-1705] 3′UTR |
| 295 | GUCUCUUUGUAUCUUCAGU | ACUGAAGAUACAAAGAGAC | | [1991-2009] 3′UTR | [1651-1669] 3′UTR |
| 296 | GACAGCAAUAUUGUUGGUC | GACCAACAAUAUUGCUGUC | | [1638-1656] ORF | — |
| 297 | UGGGACAGCAAUAUUGUUG | CAACAAUAUUGCUGUCCCA | | [1635-1653] ORF | — |
| 298 | AGAUGUGGGAUGAUCGUGA | UCACGAUCAUCCCACAUCU | | [949-967] ORF | [813-831] ORF |
| 299 | GCAAUUGGAUCACAACCUC | GAGGUUGUGAUCCAAUUGC | | [482-500] ORF | [346-384] ORF |
| 300 | CUACUACACAAGAAAAAUC | GAUUUUUCUUGUGUAGUAG | | [323-341] ORF | [187-205] ORF |
| 301 | UCCAGUGUGAACAGCAAUU | AAUUGCUGUUCACACUGGA | | [2519-2537] 3′UTR | — |
| 302 | GGUUUUCAGUUUUGUUUCU | AGAAACAAAACUGAAAACC | | [232-250] ORF | [96-114] ORF |
| 303 | CUAAGUAGGGUGACUUUUU | AAAAAGUCACCCUACUUAG | | [2367-2385] 3′UTR | — |
| 304 | UCCAGUGUUACCAUCUCCU | AGGAGAUGGUAACACUGGA | | [2299-2317] 3′UTR | — |
| 305 | CCUGGUAUUCUUUUUUUGA | UCAAAAAAAGAAUACCAGG | | [2079-2097] 3′UTR | — |
| 306 | GCAGCAUUAAACUUUGACA | UGUCAAAGUUUAAUGCUGC | GP, Rat, Ms | [1659-1677] ORF | [1376-1394] ORF |
| 307 | GCUGGACAAAUGUUCCAUU | AAUGGAACAUUUGUCCAGC | | [112-130] 5′UTR | — |
| 308 | UGAUCCUGCUUCCUGUGUG | CACACAGGAAGCAGGAUCA | | [403-421] ORF | [267-285] ORF |
| 309 | CCACAGAAGAUUUCCUAAG | CUUAGGAAAUCUUCUGUGG | | [2353-2371] 3′UTR | — |
| 310 | GGGUGGUUAACCACUGGUU | AACCAGUGGUUAACCACCC | | [217-235] ORF | [81-99] ORF |
| 311 | GCUUCAGCAACUUAAGAAC | GUUCUUAAGUUGCUGAAGC | | [2121-2139] 3′UTR | — |
| 312 | GAGUUUUCUACAAUAGCUA | UAGCUAUUGUAGAAAACUC | | [1743-1761] ORF | [1460-1478] ORF |
| 313 | UGAGUUUUCUACAAUAGCU | AGCUAUUGUAGAAAACUCA | | [1742-1760] ORF | [1459-1477] ORF |
| 314 | GGGACAAUGAGUUUUCUAC | GUAGAAAACUCAUUGUCCC | | [1735-1753] ORF | [1452-1470] ORF |
| 315 | AGGCAAAGUGGGUUUUCUA | UAGAAAACCCACUUUGCCU | | [1592-1610] ORF | — |
| 316 | GAACAGGAGAUGGAGGAAU | AUUCCUCCAUCUCCUGUUC | | [1572-1590] ORF | — |
| 317 | CCAUUCCUGAAGGACCUCU | AGAGGUCCUUCAGGAAUGG | | [126-144] 5′UTR | — |
| 318 | GACAAAUGUUCCAUUCCUG | CAGGAAUGGAACAUUUGUC | | [116-134] 5′UTR | — |
| 319 | GCCUAUCUCAUGAUGAGAA | UUCUCAUCAUGAGAUAGGC | | [634-652] ORF | [496-516] ORF |
| 320 | GUCUGCUCUCUGCUUGAAU | AUUCAAGCAGAGAGCAGAC | | [371-389] ORF | [235-253] ORF |
| 321 | CCACUGGUUUUCAGUUUUG | CAAAACUGAAAACCAGUGG | | [227-245] ORF | [91-109] ORF |
| 322 | ACUGUAGCAAGGCUUGAUA | UAUCAAGCCUUGCUACAGU | | [2226-2244] 3′UTR | — |
| 323 | UCAUGCAGCAUUAAACUUU | AAAGUUUAAUGCUGCAUGA | GP, Rat, Ms | [1655-1673] ORF | [1372-1390] ORF |
| 324 | AAAAUCUCAUAAGGGCUUU | AAAGCCCUUAUGAGAUUUU | | [1294-1312] ORF | [1158-1176] ORF |
| 325 | CCAUCCAAAGUUUUGGAAU | AUUCCAAAACUUUGGAUGG | | [1116-1134] ORF | [980-996] ORF |
| 326 | CCCUAAGUUUGAAGGGCAU | AUGCCCUUCAAACUUAGGG | | [983-1001] ORF | [847-885] ORF |
| 327 | AGCCUUGAUUCUCAUGGUA | UACCAUGAGAAUCAAGGCU | GP, Rabbit | [755-773] ORF | [619-637] ORF |
| 328 | CACCAACAGUGUACAUGUU | AACAUGUACACUGUUGGUG | | [2475-2493] 3′UTR | — |
| 329 | CGGAUGGGACAGCAAUAUU | AAUAUUGCUGUCCCAUCCG | | [1631-1649] ORF | — |
| 330 | GGAACAGGAGAUGGAGGAA | UUCCUCCAUCUCCUGUUCC | | [1571-1589] ORF | — |
| 331 | CCAUCUGGUACAAAUUCCA | UGGAAUUUGUACCAGAUGG | | [1453-1471] ORF | [1317-1335] ORF |
| 332 | CUAUCUCAUGAUGAGAAAA | UUUUCUCAUCAUGAGAUAG | | [636-654] ORF | [500-518] ORF |
| 333 | CUGCCUACAUACAGCUAUU | AAUAGCUGUAUGUAGGCAG | | [530-548] ORF | [394-412] ORF |
| 334 | UGGAUCACAACCUCACCUU | AAGGUGAGGUUGUGAUCCA | Rat, Ms | [487-505] ORF | [351-369] ORF |
| 335 | UGCUCUCUGCUUGAAUUUU | AAAAUUCAAGCAGAGAGCA | | [374-392] ORF | [238-256] ORF |
| 336 | UUCCUAAGUAGGGUGACUU | AAGUCACCCUACUUAGGAA | | [2364-2382] 3′UTR | — |
| 337 | ACCACUGGUUUUCAGUUUU | AAAACUGAAAACCAGUGGU | | [226-244] ORF | [90-108] ORF |
| 338 | GGAUCCUUUCUCAGAAAAA | UUUUUCUGAGAAAGGAUCC | | [2179-2197] 3′UTR | — |
| 339 | UGGGACAAUGAGUUUUCUA | UAGAAAACUCAUUGUCCCA | Rat, Ms | [1734-1752] ORF | [1451-1469] ORF |
| 340 | GUGGGACAAUGAGUUUUCU | AGAAAACUCAUUGUCCCAC | Rat, Ms | [1733-1751] ORF | [1450-1468] ORF |
| 341 | CCUGGUUCAACAACCUGUU | AACAGGUUGUUGAACCAGG | | [1543-1561] ORF | — |
| 342 | CCUCUCCAGAAUCCGGAUU | AAUCCGGAUUCUGGAGAGG | | [140-158] 5′UTR | [4-22] 5′UTR |
| 343 | CAUCCAAAGUUUUGGAAUU | AAUUCCAAAACUUUGGAUG | | [1117-1135] ORF | [981-999] ORF |
| 344 | CUGGUAUACUCACCACCUU | AAGGUGGUGAGUAUACCAG | | [818-836] ORF | [682-700] ORF |
| 345 | UAAGUAGGGUGACUUUUUA | UAAAAAGUCACCCUACUUA | | [2368-2388] 3′UTR | — |
| 346 | GGAUAAGAAUGUGCCUCUC | GAGAGGCACAUUCUUAUCC | | [2048-2066] 3′UTR | [1706-1726] 3′UTR |
| 347 | CCAGUCUGGAUCCUAGAAA | UUUCUAGGAUCCAGACUGG | | [1849-1867] ORF | [1566-1584] ORF |
| 348 | CGAUAUUCCAGUCUGGAUC | GAUCCAGACUGGAAUAUCG | | [1842-1860] ORF | [1559-1577] ORF |
| 349 | GAGACCAAUGUGGGACAAU | AUUGUCCCACAUUGGUCUC | Rat, Ms | [1724-1742] ORF | [1441-1459] ORF |
| 350 | CAGCAUUAAACUUUGACAA | UUGUCAAAGUUUAAUGCUG | GP | [1660-1678] ORF | [1377-1395] ORF |
| 351 | GGAAGAUUUCUUCUCCAUU | AAUGGAGAAGAAAUCUUCC | | [1247-1265] ORF | [1111-1129] ORF |
| 352 | AGGAAGAUUUCUUCUCCAU | AUGGAGAAGAAAUCUUCCU | | [1246-1264] ORF | [1110-1128] ORF |
| 353 | CCAAAGUUUUGGAAUUGCA | UGCAAUUCCAAAACUUUGG | | [1120-1138] ORF | [984-1002] ORF |
| 354 | CAAAUGUUCCAUUCCUGAA | UUCAGGAAUGGAACAUUUG | | [118-136] 5′UTR | — |
| 355 | CGCUGGACAAAUGUUCCAU | AUGGAACAUUUGUCCAGCG | | [111-129] 5′UTR | — |
| 356 | UGUGUGUUUUUCACAUCUU | AAGAUGUGAAAAACACACA | | [74-92] 5′UTR | — |
| 357 | GGAUGCCUUCCUGAAAUAU | AUAUUUCAGGAAGGCAUCC | | [287-305] ORF | [151-169] ORF |
| 358 | UGUUUCUGGUUGUUUGGUU | AACCAAACAACCAGAAACA | | [244-262] ORF | [108-126] ORF |
| 359 | CCAACAGUGUACAUGUUUA | UAAACAUGUACACUGUUGG | | [2477-2495] 3′UTR | — |
| 360 | UCCUAAGUAGGGUGACUUU | AAAGUCACCCUACUUAGGA | | [2365-2383] 3′UTR | — |
| 361 | UGGAUCCUUUCUCAGAAAA | UUUUCUGAGAAAGGAUCCA | | [2178-2196] 3′UTR | — |
| 362 | UGAAGUUCUUAAAGUUCUG | CAGAACUUUAAGAACUUCA | | [2143-2161] 3′UTR | — |
| 363 | UCACCGAUAUUCCAGUCUG | CAGACUGGAAUAUCGGUGA | | [1838-1856] ORF | [1555-1573] ORF |
| 364 | UGCAGCAUUAAACUUUGAC | GUCAAAGUUUAAUGCUGCA | GP, Rat, Ms | [1658-1675] ORF | [1375-1393] ORF |
| 365 | UGGUACAAAUUCCAGUGUG | CACACUGGAAUUUGUACCA | | [1458-1475] ORF | [1322-1340] ORF |
| 366 | CUGGUACAAAUUCCAGUGU | ACACUGGAAUUUGUACCAG | | [1457-1475] ORF | [1321-1339] ORF |
| 367 | CGAACAACAAUAUUCACCA | UGGUGAAUAUUGUUGUUCG | | [1313-1331] ORF | [1177-1195] ORF |
| 368 | ACAGUUCUGUCCAGAGAAG | CUUCUCUGGACAGAACUGU | | [10-28] 5′UTR | — |
| 369 | CCCAUCCAAAGGUUUGGAA | UUCCAAAACUUUGGAUGGG | | [1115-1133] ORF | [979-997] ORF |
| 370 | GCCCUAAGUUUGAAGGGCA | UGCCCUUCAAACUUAGGGC | | [952-1000] ORF | [846-864] ORF |
| 371 | CGAAACACGACAGUGGAGU | ACUCCACUGUCGUGUUUCG | | [687-705] ORF | [551-569] ORF |
| 372 | GGCUAAAUCCCAUCCAGUC | GACUGGAUGGGAUUUAGCC | Rat, Ms | [667-685] ORF | [531-549] ORF |
| 373 | GUGUGUGUUUUUCACAUCU | AGAUGUGAAAAACACACAC | | [73-91] 5′UTR | — |
| 374 | GGUGUGUGUUUUUCACAUC | GAUGUGAAAAACACACACC | | [72-90] 5′UTR | — |
| 375 | UCACAUCAUUGCACACCUG | CAGGUGUGCAAUGAUGUGA | | [548-568] ORF | [412-430] ORF |
| 376 | UCAUUUUGCAGCCGCACAC | GUGUGCGGCUGCAAAAUGA | | [456-474] ORF | [320-338] ORF |
| 377 | GGCACCUGCUCAUUUUGCA | UGCAAAAUGAGCAGGUGCC | GP, Rat, Ms | [447-465] ORF | [311-329] ORF |
| 378 | CCUAAGUAGGGUGACUUUU | AAAAGUCACCCUACUUAGG | | [2366-2384] 3′UTR | — |
| 379 | AACCACUGGUUUUCAGUUU | AAACUGAAAACCAGUGGUU | | [225-243] ORF | [89-107] ORF |
| 380 | AGGCUUGAUAGCAGAGGUU | AACCUCUGCUAUCAAGCCU | | [2235-2253] 3′UTR | — |
| 381 | CUAGAAAGGUUCAAUUCUA | UAGAAUUGAACCUUUCUAG | | [1861-1879] ORF | [1578-1596] ORF |
| 382 | ACAGGUCUGAAACAGAAAA | UUUUCUGUUUCAGACCUGU | GP, Rat | [1695-1713] ORF | [1412-1430] ORF |
| 383 | UUCUAAACUACCGUCUCUU | AAGAGACGGUAGUUUAGAA | Ms | [1606-1624] ORF | — |
| 384 | GGGUUUUCUAAACUACCGU | ACGGUAGUUUAGAAAACCC | | [1601-1619] ORF | — |
| 385 | CCUGUUGACUUCCCUGGAA | UUCCAGGGAAGUCAACAGG | | [1556-1574] ORF | — |
| 386 | UGAGGAUGUUUUCCAGUAU | AUACUGGAAAACAUCCUCA | Ms | [1373-1391] ORF | [1237-1255] ORF |
| 387 | CUCUCCAGAAUCCGGAUUG | CAAUCCGGAUUCUGGAGAG | | [141-159] 5′UTR | [5-23] 5′UTR |
| 388 | GCUUUCGAACAACAAUAUU | AAUAUUGUUGUUCGAAAGC | | [1308-1326] ORF | [1172-1190] ORF |
| 389 | UGGACAGAAAAUCUCAUAA | UUAUGAGAUUUUCUGUCCA | Rat | [1287-1305] ORF | [1151-1169] ORF |
| 390 | CUGGACAGAAAAUCUCAUA | UAUGAGAUUUUCUGUCCAG | Rat | [1286-1304] ORF | [1150-1168] ORF |
| 391 | UCCAUUCAUAUCCGAGCAG | CUGCUCGGAUAUGAAUGGA | | [1260-1278] ORF | [1124-1142] ORF |
| 392 | CUCCUGGAAUGGCAUCCUU | AAGGAUGCCAUUCCAGGAG | | [1206-1224] ORF | [1070-1088] ORF |
| 393 | GCACAGUUCUGUCCAGAGA | UCUCUGGACAGAACUGUGC | | [8-26] 5′UTR | — |
| 394 | GCGCUGGACAAAUGUUCCA | UGGAACAUUUGUCCAGCGC | | [110-128] 5′UTR | — |
| 395 | UCAUCCGGAGGAGUUAUUU | AAAUAACUCCUCCGGAUGA | | [790-808] ORF | [654-672] ORF |
| 396 | AAACACGACAGUGGAGUAU | AUACUCCACUGUCGUGUUU | | [689-707] ORF | [553-571] ORF |
| 397 | ACCUCACCUUCCACAAGCU | AGCUUGUGGAAGGUGAGGU | | [496-514] ORF | [360-378] ORF |
| 398 | CUACACAAGAAAAAUCCUU | AAGGAUUUUUCUUGUGUAG | | [326-344] ORF | [190-208] ORF |
| 399 | AGAUUUCCUAAGUAGGGUG | CACCCUACUUAGGAAAUCU | | [2360-2378] 3′UTR | — |
| 400 | CUCCUGAGUUUUGGUUUGU | ACAAACCAAAACUCAGGAG | | [2313-2331] 3′UTR | — |
| 401 | UGGGUGGUUAACCACUGGU | ACCAGUGGUUAACCACCCA | | [216-234] ORF | [80-98] ORF |
| 402 | GAUCCUUUCUCAGAAAAAU | AUUUUUCUGAGAAAGGAUC | | [2180-2195] 3′UTR | — |
| 403 | GAAGUUCUUAAAGUUCUGA | UCAGAACUUUAAGAACUUC | | [2144-2162] 3′UTR | — |
| 404 | GGAUCCUAGAAAGGUUCAA | UUGAACCUUUCUAGGAUCC | | [1856-1874] ORF | [1573-1591] ORF |
| 405 | UCUGGAUCCUAGAAAGGUU | AACCUUUCUAGGAUCCAGA | | [1853-1871] ORF | [1570-1588] ORF |
| 406 | GUCACCGAUAUUCCAGUCU | AGACUGGAAUAUCGGUGAC | GP | [1837-1855] ORF | [1554-1572] ORF |
| 407 | CGCAAAUGCUGUCACCGAU | AUCGGUGACAGCAUUUGCG | GP | [1827-1845] ORF | [1544-1562] ORF |
| 408 | AGGGCUUUCGAACAACAAU | AUUGUUGUUCGAAAGCCCU | | [1305-1323] ORF | [1169-1187] ORF |
| 409 | CCAUUCAUAUCCGAGCAGC | GCUGCUCGGAUAUGAAUGG | | [1261-1279] ORF | [1125-1143] ORF |
| 410 | GAAGAUUUCUUCUCCAUUC | GAAUGGAGAAGAAAUCUUC | | [1248-1266] ORF | [1112-1130] ORF |
| 411 | CUCCAGAGGAAGAUUUCUU | AAGAAAUCUUCCUCUGGAG | | [1240-1258] ORF | [1104-1122] ORF |
| 412 | GGAAUGGCAUCCUUUUACU | AGUAAAAGGAUGCCAUUCC | | [1211-1229] ORF | [1075-1093] ORF |
| 413 | UGGAAUUGCAGAUGAACAA | UUGUUCAUCUGCAAUUCCA | | [1129-1147] ORF | [993-1011] ORF |
| 414 | CACAGUUCUGUCCAGAGAA | UUCUCUGGACAGAACUGUG | | [9-27] 5′UTR | — |
| 415 | GCACCCAUCCAAAGUUUUG | CAAAACUUUGGAUGGGUGC | | [1112-1130] ORF | [976-994] ORF |
| 416 | AUGCACCCAUCCAAAGUUU | AAACUUUGGAUGGGUGCAU | | [1110-1128] ORF | [974-992] ORF |
| 417 | GGUUGUGAUUACCAAGGUU | AACCUUGGUAAUCACAACC | | [1088-1106] ORF | [952-970] ORF |
| 418 | GUGAAAGGAUCCUCCGGUU | AACCGGAGGAUCCUUUCAC | | [1051-1069] ORF | [915-933] ORF |
| 419 | CUGGACAAAUGUUCCAUUC | GAAUGGAACAUUUGUCCAG | | [113-131] 5′UTR | — |
| 420 | UUCAUCCGGAGGAGUUAUU | AAUAACUCCUCCGGAUGAA | | [789-807] ORF | [653-671] ORF |
| 421 | GCCUUGAUUCUCAUGGUAA | UUACCAUGAGAAUCAAGGC | GP, Rabbit | [756-774] ORF | [620-638] ORF |
| 422 | UGGUCUCACUGGAGUGAUC | GAUCACUCCAGUGAGACCA | | [726-746] ORF | [592-610] ORF |
| 423 | GUGUGUUUUUCACAUCUUA | UAAGAUGUGAAAAACACAC | | [75-93] 5′UTR | — |
| 424 | CGCACACUGAGAAAGCAAU | AUUGCUUUCUCAGUGUGCG | | [468-486] ORF | [332-350] ORF |
| 425 | GGCGGAAUAAACUUAUUCA | UGAAUAAGUUUAUUCCGCC | | [32-50] 5′UTR | — |
| 426 | UACAUGUUUACUGUCACUU | AAGUGACAGUAAACAUGUA | | [2486-2504] 3′UTR | — |
| 427 | CCAUAUAACACCAACAGUG | CACUGUUGGUGUUAUAUGG | | [2467-2485] 3′UTR | — |
| 428 | CCCACAGAAGAUUUCCUAA | UUAGGAAAUCUUCUGUGGG | | [2352-2370] 3′UTR | — |
| 429 | ACUAAUCCCAGGUGAUUUU | AAAAUCACCUGGGAUUAGU | | [2274-2292] 3′UTR | — |
| 430 | GACUGUAGCAAGGCUUGAU | AUCAAGCCUUGCUACAGUC | | [2225-2243] 3′UTR | — |
| 431 | AUCCUAGAAAGGUUCAAUU | AAUUGAACCUUUCUAGGAU | | [1858-1876] ORF | [1575-1593] ORF |
| 432 | CAGUCUGGAUCCUAGAAAG | CUUUCUAGGAUCCAGACUG | | [1850-1868] ORF | [1567-1585] ORF |
| 433 | GAAUCUUCCCUGUUGCCUA | UAGGCAACAGGGAAGAUUC | | [162-180] 5′UTR | [26-44] 5′UTR |
| 434 | UCCCAGGAUUGAAGUGGAU | AUCCACUUCAAUCCUGGGA | | [1334-1352] ORF | [1196-1216] ORF |
| 435 | CCAAUUCCCAGGAUUGAAG | CUUCAAUCCUGGGAAUUGG | | [1329-1347] ORF | [1193-1211] ORF |
| 436 | GGACUGGACAGAAAAUCUC | GAGAUUUUCUGUCCAGUCC | Rat | [1283-1301] ORF | [1147-1165] ORF |
| 437 | GAGGAAGAUUUCUUCUCCA | UGGAGAAGAAAUCUUCCUC | | [1245-1263] ORF | [1109-1127] ORF |
| 438 | CCAGAGGAAGAUUUCUUCU | AGAAGAAAUCUUCCUCUGG | | [1242-1260] ORF | [1106-1124] ORF |
| 439 | CGCAAGUGUGCAGAGUCUU | AAGACUCUGCACACUUGCG | | [927-945] ORF | [791-809] ORF |
| 440 | GAGCAUGAAUGAGAGUCAU | AUGACUCUCAUUCAUGCUC | | [905-923] ORF | [769-787] ORF |
| 441 | GGUAUACUCACCACCUUUU | AAAAGGUGGUGAGUAUACC | | [820-838] ORF | [684-702] ORF |
| 442 | CAUGACAAUAGCCUUGAUU | AAUCAAGGCUAUUGUCAUG | | [746-764] ORF | [610-628] ORF |
| 443 | UCAUGACAAUAGCCUUGAU | AUCAAGGCUAUUGUCAUGA | | [745-763] ORF | [609-627] ORF |
| 444 | UGAUCAUGACAAUAGCCUU | AAGGCUAUUGUCAUGAUCA | | [742-760] ORF | [606-624] ORF |
| 445 | GUCUCACUGGAGUGAUCAU | AUGAUCACUCCAGUGAGAC | | [730-748] ORF | [594-612] ORF |
| 446 | GAGUAUGUGACAUUCACCA | UGGUGAAUGUCACAUACUC | | [702-720] ORF | [566-584] ORF |
| 447 | CAGCCUAUCUCAUGAUGAG | CUCAUCAUGAGAUAGGCUG | | [632-650] ORF | [496-514] ORF |
| 448 | AGCAAUUGGAUCACAACCU | AGGUUGUGAUCCAAUUGCU | | [481-499] ORF | [345-383] ORF |
| 449 | ACUUAUUCAUUCCCAGGAA | UUCCUGGGAAUGAAUAAGU | | [42-60] 5′UTR | — |
| 450 | UCAGUUUUGUUUCUGGUUG | CAACCAGAAACAAAACUGA | | [237-255] ORF | [101-119] ORF |
| 451 | UCCUGAGUUUUGGUUUGUA | UACAAACCAAAACUCAGGA | | [2314-2332] 3′UTR | — |
| 452 | GAACUUCUGAAGUUCUUAA | UUAAGAACUUCAGAAGUUC | | [2136-2154] 3′UTR | — |
| 453 | GAUCCUAGAAAGGUUCAAU | AUUGAACCUUUCUAGGAUC | | [1857-1875] ORF | [1574-1592] ORF |
| 454 | CCGAUAUUCCAGUCUGGAU | AUCCAGACUGGAAUAUCGG | | [1841-1859] ORF | [1558-1576] ORF |
| 455 | AGACCAAUGUGGGACAAUG | CAUUGUCCCACAUUGGUCU | Rat, Ms | [1725-1743] ORF | [1442-1460] ORF |
| 456 | GAAUCCGGAUUGCUGAAUC | GAUUCAGCAAUCCGGAUUC | | [148-166] 5′UTR | [12-30] 5′UTR |
| 457 | CCAGUAUGAAGUGGCUGUG | CACAGCCACUUCAUACUGG | | [1385-1403] ORF | [1249-1267] ORF |
| 458 | UUCACCAAUUCCCAGGAUU | AAUCCUGGGAAUUGGUGAA | | [1325-1343] ORF | [1189-1207] ORF |
| 459 | CAGAGGAAGAUUUCUUCUC | GAGAAGAAAUCUUCCUCUG | | [1243-1261] ORF | [1107-1125] ORF |
| 460 | CUGGAAUGGCAUCCUUUUA | UAAAAGGAUGCCAUUCCAG | | [1209-1227] ORF | [1073-1091] ORF |
| 461 | CCUGGAAUGGCAUCCUUUU | AAAAGGAUGCCAUUCCAGG | | [1208-1226] ORF | [1072-1090] ORF |
| 462 | UGCACCCAUCCAAAGUUUU | AAAACUUUGGAUGGGUGCA | | [1111-1129] ORF | [975-993] ORF |
| 463 | UGAGAGUCAUCCUCGCAAG | CUUGCGAGGAUGACUCUCA | | [914-932] ORF | [778-795] ORF |
| 464 | CUCAUGGUAACUUCAGCUA | UAGCUGAAGUUACCAUGAG | GP, Rat | [765-783] ORF | [629-647] ORF |
| 465 | AGGUGUGUGUUUUUCACAU | AUGUGAAAAACACACACCU | | [71-89] 5′UTR | — |
| 466 | GCCUACAUACAGCUAUUCA | UGAAUAGCUGUAUGUAGGC | | [532-550] ORF | [396-414] ORF |
| 467 | GGCCUAUAUGAUCUGCCUA | UAGGCAGAUCAUAUAGGCC | | [518-536] ORF | [382-400] ORF |
| 468 | UCACCUUCCACAAGCUGGU | ACCAGCUUGUGGAAGGUGA | | [499-517] ORF | [363-381] ORF |
| 469 | UGCUCAUUUUGCAGCCGCA | UGCGGCUGCAAAAUGAGCA | | [453-471] ORF | [317-335] ORF |
| 470 | GCACCUGCUCAUUUUGCAG | CUGCAAAAUGAGCAGGUGC | | [448-466] ORF | [312-330] ORF |
| 471 | UGGGUCAACAUUGGCCUGU | ACAGGCCAAUGUUGACCCA | | [344-362] ORF | [205-226] ORF |
| 472 | ACAGUGUACAUGUUUACUG | CAGUAAACAUGUACACUGU | | [2480-2498] 3′UTR | — |
| 473 | CAUCUCCUGAGUUUUGGUU | AACCAAAACUCAGGAGAUG | | [2310-2328] 3′UTR | — |
| 474 | CUAAUCCCAGGUGAUUUUA | UAAAAUCACCUGGGAUUAG | | [2275-2293] 3′UTR | — |
| 475 | CCAUGACUGUAGCAAGGCU | AGCCUUGCUACAGUCAUGG | | [2221-2239] 3′UTR | — |
| 476 | UGGACAGCCAUGACUGUAG | CUACAGUCAUGGCUGUCCA | | [2214-2232] 3′UTR | — |
| 477 | GAAUGUGCCUCUCAAGCCU | AGGCUUGAGAGGCACAUUC | | [2054-2072] 3′UTR | [1714-1732] 3′UTR |
| 478 | CCUCGGACUUUGGCAAAGA | UCUUUGCCAAAGUCCGAGG | | [1603-1621] ORF | [1520-1538] ORF |
| 479 | CAGGUCUGAAACAGAAAAC | GUUUUCUGUUUCAGACCUG | GP, Rat | [1696-1714] ORF | [1413-1431] ORF |
| 480 | GGUUCAACAACCUGUUGAC | GUCAACAGGUUGUUGAACC | | [1546-1564] ORF | — |
| 481 | GCUGGUUGGAGCAGGAAUU | AAUUCCUGCUCCAACCAGC | | [1403-1421] ORF | [1267-1285] ORF |
| 482 | CUGAAGGACCUCUCCAGAA | UUCUGGAGAGGUCCUUCAG | | [132-150] 5′UTR | [1-14] (14/14) |
| | | | | | 5′UTR |
| 483 | AGAUGAACAAGCGUGGCUU | AAGCCACGCUUGUUCAUCU | | [1138-1156] ORF | [1002-1020] ORF |
| 484 | AGGUUGUGAUUACCAAGGU | ACCUUGGUAAUCACAACCU | | [1087-1105] ORF | [951-969] ORF |
| 485 | GCAGAAGGUUGUGAUUACC | GGUAAUCACAACCUUCUGC | | [1082-1100] ORF | [946-964] ORF |
| 486 | AAAGGAUCCUCCGGUUUUA | UAAAACCGGAGGAUCCUUU | | [1054-1072] ORF | [918-936] ORF |
| 487 | UCGCAAGUGUGCAGAGUCU | AGACUCUGCACACUUGCGA | | [926-944] ORF | [790-808] ORF |
| 488 | CCUGCGCUGGACAAAUGUU | AACAUUUGUCCAGCGCAGG | | [107-125] 5′UTR | — |
| 489 | CCUUGAUUCUCAUGGUAAC | GUUACCAUGAGAAUCAAGG | GP, Rabbit | [757-775] ORF | [621-639] ORF |
| 490 | UGGAGUAUGUGACAUUCAC | GUGAAUGUCACAUACUCCA | | [700-718] ORF | [564-582] ORF |
| 491 | CUAUAGCAGAAGCCGACAG | CUGUCGGCUUCUGCUAUAG | | [581-599] ORF | [445-463] ORF |
| 492 | CAAGAAAAAUCCUUGGGUC | GACCCAAGGAUUUUUCUUG | | [331-349] ORF | [195-213] ORF |
| 493 | ACAAGAAAAAUCCUUGGGU | ACCCAAGGAUUUUUCUUGU | | [330-348] ORF | [194-212] ORF |
| 494 | UCCUGUUUGUGGAUGCCUU | AAGGCAUCCACAAACAGGA | | [277-295] ORF | [141-159] ORF |
| 495 | CAGUGUACAUGUUUACUGU | ACAGUAAACAUGUACACUG | | [2481-2499] 3′UTR | — |
| 496 | CAACUAAUCCCAGGUGAUU | AAUCACCUGGGAUUAGUUG | | [2272-2290] 3′UTR | — |
| 497 | GGCUUGAUAGCAGAGGUUU | AAACCUCUGCUAUCAAGCC | | [2236-2254] 3′UTR | — |
| 498 | AGCAAGGCUUGAUAGCAGA | UCUGCUAUCAAGCCUUGCU | | [2231-2249] 3′UTR | — |
| 499 | AGCCUUGACUCCCUGGUAU | AUACCAGGGAGUCAAGGCU | | [2068-2086] 3′UTR | — |
| 500 | GACAGGUCUGAAACAGAAA | UUUCUGUUUCAGACCUGUC | GP, Rat | [1694-1712] ORF | [1411-1429] ORF |
| 501 | UGACAGGUCUGAAACAGAA | UUCUGUUUCAGACCUGUCA | GP, Rat | [1693-1711] ORF | [1410-1428] ORF |
| 502 | UCUUCCCUGUUGCCUAGAA | UUCUAGGCAACAGGGAAGA | | [165-183] 5′UTR | [29-47] 5′UTR |
| 503 | GUCAUGCAGCAUUAAACUU | AAGUUUAAUGCUGCAUGAC | GP, Rat, Ms | [1654-1672] ORF | [1371-1389] ORF |
| 504 | CAGGUGCCUUUUCCUGGUU | AACCAGGAAAAGGCACCUG | GP | [1531-1549] ORF | — |
| 505 | CCGGAUUGCUGAAUCUUCC | GGAAGAUUCAGCAAUCCGG | | [152-170] 5′UTR | [16-34] 5′UTR |
| 506 | GUGCAGACCACAACCUCAA | UUGAGGUUGUGGUCUGCAC | GP | [1474-1492] ORF | [1338-1356] ORF |
| 507 | UUGCUUCUAUCUUGAAAUC | GAUUUCAAGAUAGAAGCAA | | [1435-1453] ORF | [1299-1317] ORF |
| 508 | CCAGAAUCCGGAUUGCUGA | UCAGCAAUCCGGAUUCUGG | | [145-163] 5′UTR | [9-27] 5′UTR |
| 509 | GAGGAUGUUUUCCAGUAUG | CAUACUGGAAAACAUCCUC | Ms | [1374-1392] ORF | [1238-1256] ORF |
| 510 | ACUGGACAGAAAAUCUCAU | AUGAGAUUUUCUGUCCAGU | Rat | [1285-1303] ORF | [1149-1167] ORF |
| 511 | AAGAUUUCUUCUCCAUUCA | UGAAUGGAGAAGAAAUCUU | | [1249-1267] ORF | [1113-1131] ORF |
| 512 | AGAGGAAGAUUUCUUCUCC | GGAGAAGAAAUCUUCCUCU | | [1244-1262] ORF | [1108-1126] ORF |
| 513 | UCCAGAGGAAGAUUUCUUC | GAAGAAAUCUUCCUCUGGA | | [1241-1259] ORF | [1105-1123] ORF |
| 514 | CUCUGCUCCAGAGGAAGAU | AUCUUCCUCUGGAGCAGAG | | [1235-1253] ORF | [1099-1117] ORF |
| 515 | UCCUGGAAUGGCAUCCUUU | AAAGGAUGCCAUUCCAGGA | | [1207-1225] ORF | [1071-1089] ORF |
| 516 | GGAAUUGCAGAUGAACAAG | CUUGUUCAUCUGCAAUUCC | | [1130-1148] ORF | [994-1012] ORF |
| 517 | AUCCAAAGUUUUGGAAUUG | CAAUUCCAAAACUUUGGAU | | [1116-1136] ORF | [962-1000] ORF |
| 518 | GAUCCUUGCACCGGUCAUU | AAUGACCGGUGCAAGGAUC | | [1022-1040] ORF | [886-904] ORF |
| 519 | GAGUCAUCCUCGCAAGUGU | ACACUUGCGAGGAUGACUC | | [917-935] ORF | [781-799] ORF |
| 520 | UCACUGGAGUGAUCAUGAC | GUCAUGAUCACUCCAGUGA | | [733-751] ORF | [597-615] ORF |
| 521 | GUAGGUGUGUGUUUUUCAC | GUGAAAAACACACACCUAC | | [69-87] 5′UTR | — |
| 522 | UGCCUACAUACAGCUAUUC | GAAUAGCUGUAUGUAGGCA | | [531-549] ORF | [395-413] ORF |
| 523 | CCGCACACUGAGAAAGCAA | UUGCUUUCUCAGUGUGCGG | | [467-485] ORF | [331-349] ORF |
| 524 | GUGGUUAACCACUGGUUUU | AAAACCAGUGGUUAACCAC | | [219-237] ORF | [83-101] ORF |
| 525 | GGUGGUUAACCACUGGUUU | AAACCAGUGGUUAACCACC | | [218-236] ORF | [82-100] ORF |
| 526 | GCAAGGCUUGAUAGCAGAG | CUCUGCUAUCAAGCCUUGC | | [2232-2250] 3′UTR | — |
| 527 | CUUAAGAACUUCUGAAGUU | AACUUCAGAAGUUCUUAAG | | [2131-2149] 3′UTR | — |
| 528 | CGUUACUUGAGCUUCAGCA | UGCUGAAGCUCAAGUAACG | | [2111-2129] 3′UTR | — |
| 529 | GGUUCAAUUCUACUUCAAC | GUUGAAGUAGAAUUGAACC | Rat | [1868-1886] ORF | [1585-1603] ORF |
| 530 | CCAAUGUGGGACAAUGAGU | ACUCAUUGUCCCACAUUGG | Rat, Ms | [1728-1746] ORF | [1445-1463] ORF |
| 531 | UCAACAACCUGUUGACUUC | GAAGUCAACAGGUUGUUGA | | [1549-1567] ORF | — |
| 532 | UGGUUCAACAACCUGUUGA | UCAACAGGUUGUUGAACCA | | [1545-1563] ORF | — |
| 533 | GCUGAUAGCACAGUUCUGU | ACAGAACUGUGCUAUCAGC | | [1-91] 5′UTR | — |
| 534 | CUGCUCCAGAGGAAGAUUU | AAAUCUUCCUCUGGAGCAG | | [1237-1255] ORF | [1101-1119] ORF |
| 535 | UCCAAAGUUUUGGAAUUGC | GCAAUUCCAAAACUUUGGA | | [1119-1137] ORF | [963-1001] ORF |
| 536 | CCAAGGUUGUUAUGCACCC | GGGUGCAUAACAACCUUGG | | [1099-1117] ORF | [963-961] ORF |
| 537 | GCAUGAAUGAGAGUCAUCC | GGAUGACUCUCAUUCAUGC | | [907-925] ORF | [771-789] ORF |
| 538 | UGGCCUAUAUGAUCUGCCU | AGGCAGAUCAUAUAGGCCA | | [517-535] ORF | [381-399] ORF |
| 539 | GCUUGAAUUUUAACAGCAC | GUGCUGUUAAAAUUCAAGC | | [382-400] ORF | [248-264] ORF |
| 540 | UGCUUGAAUUUUAACAGCA | UGCUGUUAAAAUUCAAGCA | Ms | [381-399] ORF | [245-263] ORF |
| 541 | CUCUGCUUGAAUUUUAACA | UGUUAAAAUUCAAGCAGAG | | [378-396] ORF | [242-260] ORF |
| 542 | AGGCGGAAUAAACUUAUUC | GAAUAAGUUUAUUCCGCCU | | [31-49] 5′UTR | — |
| 543 | CACUGGUUUUCAGUUUUGU | ACAAAACUGAAAACCAGUG | | [228-248] ORF | [92-110] ORF |
| 544 | AUGGAUCCUUUCUCAGAAA | UUUCUGAGAAAGGAUCCAU | | [2177-2195] 3′UTR | — |
| 545 | GAAACUGGGUGGUUAACCA | UGGUUAACCACCCAGUUUC | | [211-229] ORF | [75-93] ORF |
| 546 | UUGAGCUUCAGCAACUUAA | UUAAGUUGCUGAAGCUCAA | | [2117-2135] 3′UTR | — |
| 547 | CCACCUCUUGACAAUGGGA | UCCCAUUGUCAAGAGGUGG | | [194-212] | [58-76] |
| | | | | 5′UTR + ORF | 5′UTR + ORF |
| 548 | CUGGAUCCUAGAAAGGUUC | GAACCUUUCUAGGAUCCAG | | [1854-1872] ORF | [1571-1589] ORF |
| 549 | GGACUUUGGCAAAGAGCCU | AGGCUCUUUGCCAAAGUCC | | [1807-1825] ORF | [1524-1542] ORF |
| 550 | UGAAACAGAAAACCUCCUU | AAGGAGGUUUUCUGUUUCA | Rat | [1702-1720] ORF | [1419-1437] ORF |
| 551 | CUGAAACAGAAAACCUCCU | AGGAGGUUUUCUGUUUCAG | Rat | [1701-1719] ORF | [1418-1436] ORF |
| 552 | CUGUUGACUUCCCUGGAAC | GUUCCAGGGAAGUCAACAG | | [1557-1575] ORF | — |
| 553 | CCAGUGAGGAUGUUUUCCA | UGGAAAACAUCCUCACUGG | | [1369-1387] ORF | [1233-1251] ORF |
| 554 | CAGCCAGUGAGGAUGUUUU | AAAACAUCCUCACUGGCUG | | [1368-1384] ORF | [1230-1248] ORF |
| 555 | CACAGCCAGUGAGGAUGUU | AACAUCCUCACUGGCUGUG | | [1364-1382] ORF | [1228-1246] ORF |
| 556 | ACCAAUUCCCAGGAUUGAA | UUCAAUCCUGGGAAUUGGU | | [1328-1346] ORF | [1192-1210] ORF |
| 557 | UGGAAUGGCAUCCUUUUAC | GUAAAAGGAUGCCAUUCCA | | [1210-1228] ORF | [1074-1092] ORF |
| 558 | ACCCAUCCAAAGUUUUGGA | UCCAAAACUUUGGAUGGGU | | [1114-1132] ORF | [978-996] ORF |
| 559 | CAAGGUUGUUAUGCACCCA | UGGGUGCAUAACAACCUUG | | [1100-1118] ORF | [964-982] ORF |
| 560 | UGGUAUACUCACCACCUUU | AAAGGUGGUGAGUAUACCA | | [819-837] ORF | [683-701] ORF |
| 561 | GUGACAUUCACCAGCGUUG | CAACGCUGGUGAAUGUCAC | | [708-726] ORF | [572-586] (15/15) |
| | | | | | ORF |
| 562 | UCUGCCUACAUACAGCUAU | AUAGCUGUAUGUAGGCAGA | | [529-547] ORF | [393-411] ORF |
| 563 | CUUUUGAUAUGGUCUUAUC | GAUAAGACCAUAUCAAAAG | | [2502-2520] 3′UTR | — |
| 564 | UGGUUAACCACUGGUUUUC | GAAAACCAGUGGUUAACCA | | [220-238] ORF | [84-102] ORF |
| 565 | CAUGGAUCCUUUCUCAGAA | UUCUGAGAAAGGAUCCAUG | | [2176-2194] 3′UTR | — |
| 566 | AGAACUUGUGAAGUUCUUA | UAAGAACUUCAGAAGUUCU | | [2135-2153] 3′UTR | — |
| 567 | AAGAACUUCUGAAGUUCUU | AAGAACUUCAGAAGUUCUU | | [2134-2152] 3′UTR | — |
| 568 | CUCCAAACCACCUCUUGAC | GUCAAGAGGUGGUUUGGAG | | [187-205] 5′UTR | [51-69] 5′UTR |
| 569 | UGCCUAGAAGGGCUCCAAA | UUUGGAGCCCUUCUAGGCA | | [175-193] 5′UTR | [39-57] 5′UTR |
| 570 | CGUGACAGGUCUGAAACAG | CUGUUUCAGACCUGUCACG | | [1691-1709] ORF | [1408-1426] ORF |
| 571 | CUGACAUCGUGACAGGUCU | AGACCUGUCACGAUGUCAG | | [1684-1702] ORF | [1401-1419] ORF |
| 572 | UGACUUCCCUGGAACAGGA | UCCUGUUCCAGGGAAGUCA | | [1561-1579] ORF | — |
| 573 | UCCAGUAUGAAGUGGCUGU | ACAGCCACUUCAUACUGGA | Ms | [1384-1402] ORF | [1248-1266] ORF |
| 574 | CAGUGAGGAUGUUUUCCAG | CUGGAAAACAUCCUCACUG | Ms | [1370-1388] ORF | [1234-1252] ORF |
| 575 | AGGUUGUUAUGCACCCAUC | GAUGGGUGCAUAACAACCU | | [1102-1120] ORF | [966-984] ORF |
| 576 | AAGGUUGUUAUGCACCCAU | AUGGGUGCAUAACAACCUU | | [1101-1119] ORF | [965-983] ORF |
| 577 | CCCAGCAGAAGGUUGUGAU | AUCACAACCUUCUGCUGGG | | [1078-1096] ORF | [942-960] ORF |
| 578 | CCUUGCACCGGUCAUUCUU | AAGAAUGACCGGUGCAAGG | | [1025-1043] ORF | [889-907] ORF |
| 579 | GUGAUCAUGACAAUAGCCU | AGGCUAUUGUCAUGAUCAC | | [741-759] ORF | [605-623] ORF |
| 580 | CUGGUCUCACUGGAGUGAU | AUCACUCCAGUGAGACCAG | | [727-745] ORF | [591-609] ORF |
| 581 | UGCUUCCUGUGUGUCGCAA | UUGCGACACACAGGAAGCA | | [409-427] ORF | [273-291] ORF |
| 582 | CUGCUUGAAUUUUAACAGC | GCUGUUAAAAUUCAAGCAG | | [380-398] ORF | [244-262] ORF |
| 583 | CCAUCUCCUGAGUUUUGGU | ACCAAAACUCAGGAGAUGG | | [2309-2327] 3′UTR | — |
| 584 | CAUGACUGUAGCAAGGCUU | AAGCCUUGCUACAGUCAUG | | [2222-2240] 3′UTR | — |
| 585 | ACUUGAGCUUCAGCAACUU | AAGUUGCUGAAGCUCAAGU | | [2115-2133] 3′UTR | — |
| 586 | CCUAGAAAGGUUCAAUUCU | AGAAUUGAACCUUUCUAGG | | [1860-1878] ORF | [1577-1595] ORF |
| 587 | UCCAGUCUGGAUCCUAGAA | UUCUAGGAUCCAGACUGGA | | [1848-1866] ORF | [1565-1583] ORF |
| 588 | AGGUGCCUUUUCCUGGUUC | GAACCAGGAAAAGGCACCU | GP | [1532-1550] ORF | — |
| 589 | ACAGGUGCCUUUUCCUGGU | ACCAGGAAAAGGCACCUGU | GP | [1530-1548] ORF | — |
| 590 | GGAUUGAAGUGGAUGGUCC | GGACCAUCCACUUCAAUCC | | [1339-1357] ORF | [1203-1221] ORF |
| 591 | UGGAAGUGGAUCCUUGCAC | GUGCAAGGAUCCACUUCCA | Ms | [1014-1032] ORF | [878-896] ORF |
| 592 | UGACAAUAGCCUUGAUUCU | AGAAUCAAGGCUAUUGUCA | | [748-766] ORF | [612-630] ORF |
| 593 | GUUCUUGGCUAAAUCCCAU | AUGGGAUUUAGCCAAGAAC | | [661-679] ORF | [525-543] ORF |
| 594 | GUCUUAUCCAGUGUGAACA | UGUUCACACUGGAUAAGAG | | [2513-2531] 3′UTR | — |
| 595 | UGGUCUUAUCCAGUGUGAA | UUCACACUGGAUAAGACCA | | [2511-2529] 3′UTR | — |
| 596 | CCAGUGUUACCAUCUCCUG | CAGGAGAUGGUAACACUGG | | [2300-2318] 3′UTR | — |
| 597 | AGCUUCAGCAACUUAAGAA | UUCUUAAGUUGCUGAAGCU | | [2120-2138] 3′UTR | — |
| 598 | CCUCCUUUGGGAGACCAAU | AUUGGUCUCCCAAAGGAGG | Rat | [1714-1732] ORF | [1431-1449] ORF |
| 599 | ACAGCCAGUGAGGAUGUUU | AAACAUCCUCACUGGCUGU | | [1365-1383] ORF | [1229-1247] ORF |
| 600 | UCCUUGGCUUAGGGAUUCA | UGAAUCCCUAAGCCAAGGA | | [850-868] ORF | [714-732] ORF |
| 601 | CACAAGCUGGUGGCCUAUA | UAUAGGCCACCAGCUUGUG | | [507-525] ORF | [371-389] ORF |
| 602 | ACCAACAGUGUACAUGUUU | AAACAUGUACACUGUUGGU | | [2476-2494] 3′UTR | — |
| 603 | UGCCUCUCAAGCCUUGACU | AGUCAAGGCUUGAGAGGCA | | [2059-2077] 3′UTR | [1719-1734] (16/16) |
| | | | | | 3′UTR |
| 604 | ACCUCCUUUGGGAGACCAA | UUGGUCUCCCAAAGGAGGU | Rat | [1713-1731] ORF | [1430-1448] ORF |
| 605 | CAGAAAACCUCCUUUGGGA | UCCCAAAGGAGGUUUUCUG | Rat | [1707-1725] ORF | [1424-1442] ORF |
| 606 | ACCGGAUGGGACAGCAAUA | UAUUGCUGUCCCAUCCGGU | | [1629-1647] ORF | — |
| 607 | CCUUGGCUUAGGGAUUCAC | GUGAAUCCCUAAGCCAAGG | | [851-869] ORF | [715-733] ORF |
| 608 | CUGGUUUUCAGUUUUGUUU | AAACAAAACUGAAAACCAG | | [230-248] ORF | [94-112] ORF |
| 609 | CAACAGUGUACAUGUUUAC | GUAAACAUGUACACUGUUG | | [2478-2496] 3′UTR | — |
| 610 | AGGUGAUUUUAUCAAUUCC | GGAAUUGAUAAAAUCACCU | | [2283-2301] 3′UTR | — |
| 611 | UAAAGCCCAUGGAUCCUUU | AAAGGAUCCAUGGGCUUUA | | [2169-2187] 3′UTR | — |
| 612 | GUCUGAAACAGAAAACCUC | GAGGUUUUCUGUUUCAGAC | Rat | [1699-1717] ORF | [1416-1434] ORF |
| 613 | GUCUUGGAAGUGGAUCCUU | AAGGAUCCACUUCCAAGAC | Ms | [1010-1026] ORF | [874-892] ORF |
| 614 | GGAUGUUUUCCAGUAUGAA | UUCAUACUGGAAAACAUCC | Ms | [1376-1394] ORF | [1240-1258] ORF |
| 615 | UCCUUUGGGAGACCAAUGU | ACAUUGGUCUCCCAAAGGA | Rat, Ms | [1716-1734] ORF | [1433-1451] ORF |
| 616 | ACAGCCAUGACUGUAGCAA | UUGCUACAGUCAUGGCUGU | | [2217-2235] 3′UTR | — |
| 617 | AGGGAGACAGGUGCCUUUU | AAAAGGCACCUGUCUCCCU | GP | [1524-1542] ORF | — |
| 618 | AGGAUGUUUUCCAGUAUGA | UCAUACUGGAAAACAUCCU | Ms | [1375-1393] ORF | [1239-1257] ORF |
| 619 | GGUAACUUCAGCUACUGAG | CUCAGUAGCUGAAGUUACC | | [770-788] ORF | [634-652] ORF |

TABLE D
21-mer oligomers for NOX1 (NADPH oxidase 1)

| | | | | | |
|---|---|---|---|---|---|
| | | | | Human-21614529 | Human-7669509 |
| Number | Sense siRNA | AntiSense siRNA | Other Sp | ORF: 207-1901 | ORF: 71-1618 |

| | | | | | |
|---|---|---|---|---|---|
| 1 | GCAGUCACUUUAGGAUAAGAA | UUCUUAUCCUAAAGUGACUGC | | [2036-2056] 3′UTR | [1696-1716] 3′UTR |
| 2 | GGACAAUGAGUUUUCUACAAU | AUUGUAGAAAACUCAUUGUCC | | [1736-1756] ORF | [1453-1473] ORF |
| 3 | CAGUCACUUUAGGAUAAGAAU | AUUCUUAUCCUAAAGUGACUG | | [2037-2057] 3′UTR | [1697-1717] 3′UTR |
| 4 | GCAAAGUGGGUUUUCUAAACU | AGUUUAGAAAACCCACUUUGC | | [1594-1614] ORF | — |
| 5 | GGGCUGAAUGUUUUCCUGUUU | AAACAGGAAAACAUUCAGCCC | | [264-284] ORF | [128-148] ORF |
| 6 | ACAUGUUUACUGUGACUUUUG | CAAAAGUGACAGUAAACAUGU | | [2487-2507] 3′UTR | — |
| 7 | GAAAUCCAUCUGGUACAAAUU | AAUUUGUACCAGAUGGAUUUC | | [1448-1468] ORF | [1312-1332] ORF |
| 8 | CCUCAAUCUCUCUCCUGGAAU | AUUCCAGGAGAGAGAUUGAGG | | [1195-1215] ORF | [1059-1079] ORF |
| 9 | GCACACCUGUUUAACUUUGAC | GUCAAAGUUAAACAGGUGUGC | | [558-578] ORF | [422-442] ORF |
| 10 | ACAUCAUUGCACACCUGUUUA | UAAACAGGUGUGCAAUGAUGU | | [550-570] ORF | [414-434] ORF |
| 11 | GCGUCUGCUCUCUGCUUGAAU | AUUCAAGCAGAGAGCAGACGC | | [369-389] ORF | [233-253] ORF |
| 12 | CAAAGUGGGUUUUCUAAACUA | UAGUUUAGAAAACCCACUUUG | | [1595-1615] ORF | — |
| 13 | CCACAACCUCAAAACAAAAAA | UUUUUUGUUUUGAGGUUGUGG | | [1481-1501] ORF | [1345-1365] ORF |
| 14 | GGGCAGUAUAUCUUUGUUAAU | AUUAACAAAGAUAUACUGCCC | | [1170-1190] ORF | [1034-1054] ORF |
| 15 | CCGGUCAUUCUUUAUAUCUGU | ACAGAUAUAAAGAAUGACCGG | | [1032-1052] ORF | [898-918] ORF |
| 16 | UGCAUUCAACUUCGUUACUUG | CAAGUAACGAAGUUGAAUGCA | | [2099-2119] 3′UTR | — |
| 17 | GCAAAGGCUGUCACCGAUAUU | AAUAUCGGUGACAGCAUUUGC | GP | [1828-1848] ORF | [1545-1565] ORF |
| 18 | CCAAGUCUGUAGUGGGAGUUU | AAACUCCCACUACAGACUUGG | | [1771-1791] ORF | [1488-1508] ORF |
| 19 | GCACCGGUCAUUCUUUAUAUC | GAUAUAAAGAAUGACCGGUGC | | [1029-1049] ORF | [893-913] ORF |
| 20 | ACAUACAGCUAUUCACAUCAU | AUGAUGUGAAUAGCUGUAUGU | | [536-556] ORF | [400-420] ORF |
| 21 | GCCUAUAUGAUCUGCCUACAU | AUGUAGGCAGAUCAUAUAGGC | | [519-539] ORF | [383-403] ORF |
| 22 | GGAUCACAACCUCACCUUCCA | UGGAAGGUGAGGUUGUGAUCC | Rat, Ms | [488-508] ORF | [352-372] ORF |
| 23 | CGACAAAUACUACUACACAAG | CUUGUGUAGUAGUAUUUGUCG | | [314-334] ORF | [178-198] ORF |
| 24 | GGAGUUUUCUUAUGUGGCCCU | AGGGCCACAUAAGAAAACUCC | | [1785-1805] ORF | [1502-1522] ORF |
| 25 | GGAAUUAGGCAAAGUGGGUUU | AAACCCACUUUGCCUAAUUCC | | [1586-1606] ORF | — |
| 26 | GCAGACCACAACCUCAAAACA | UGUUUUGAGGUUGUGGUCUGC | | [1476-1496] ORF | [1340-1360] ORF |
| 27 | CAAAUUCCAGUGUGCAGACCA | UGGUCUGCACACUGGAAUUUG | | [1463-1483] ORF | [1327-1347] ORF |
| 28 | GGCUUAGGGAUUCACGGCAUU | AAUGCCGUGAAUCCCUAAGCC | | [855-875] ORF | [719-739] ORF |
| 29 | GGAGUUAUUUUGAAGUCUUCU | AGAAGACUUCAAAAUAACUCC | | [799-819] ORF | [663-683] ORF |
| 30 | GACAGUGGAGUAUGUGACAUU | AAUGUCACAUACUCCACUGUC | | [695-715] ORF | [559-579] ORF |
| 31 | UGCACACCUGUUUAACUUUGA | UCAAAGUUAAACAGGUGUGCA | | [557-577] ORF | [421-441] ORF |
| 32 | GCUAUUCACAUCAUUGCACAC | GUGUGCAAUGAUGUGAAUAGC | | [543-563] ORF | [407-427] ORF |
| 33 | AGCAGUCACUUUAGGAUAAGA | UCUUAUCCUAAAGUGACUGCU | | [2035-2055] 3′UTR | [1695-1715] 3′UTR |
| 34 | GAGCAGUCACUUUAGGAUAAG | CUUAUCCUAAAGUGACUGCUC | | [2034-2054] 3′UTR | [1694-1714] 3′UTR |
| 35 | CUGUAGUGGGAGUUUUCUUAU | AUAAGAAAACUCCCACUACAG | | [1777-1797] ORF | [1494-1514] ORF |
| 36 | GACCACAACCUCAAAACAAAA | UUUUGUUUUGAGGUUGUGGUC | | [1479-1499] ORF | [1343-1363] ORF |
| 37 | CGGUCAUUCUUUAUAUCUGUG | CACAGAUAUAAAGAAUGACCG | | [1033-1053] ORF | [897-917] ORF |
| 38 | UGCACCGGUCAUUCUUUAUAU | AUAUAAAGAAUGACCGGUGCA | | [1028-1048] ORF | [892-912] ORF |
| 39 | GAGGAGUUAUUUUGAAGUCUU | AAGACUUCAAAAUAACUCCUC | | [797-817] ORF | [661-681] ORF |
| 40 | GGAGGAGUUAUUUUGAAGUCU | AGACUUCAAAAUAACUCCUCC | | [796-816] ORF | [660-680] ORF |
| 41 | CACAUCAUUGCACACCUGUUU | AAACAGGUGUGCAAUGAUGUG | | [549-569] ORF | [413-433] ORF |
| 42 | CAUACAGCUAUUCACAUCAUU | AAUGAUGUGAAUAGCUGUAUG | | [537-557] ORF | [401-421] ORF |
| 43 | GAGAAAGCAAUUGGAUCACAA | UUGUGAUCCAAUUGCUUUCUC | | [476-496] ORF | [340-360] ORF |
| 44 | GAAAUAUGAGAAGGCCGACAA | UUGUCGGCCUUCUCAUAUUUC | | [299-319] ORF | [163-183] ORF |
| 45 | GGAAGGCGGAAUAAACUUAUU | AAUAAGUUUAUUCCGCCUUCC | | [28-48] 5′UTR | — |
| 46 | GGCUGAAUGUUUUCCUGUUUG | CAAACAGGAAAACAUUCAGCC | | [265-285] ORF | [129-149] ORF |
| 47 | UGGUUCAGAGUUAUACAACUA | UAGUUGUAUAACUCUGAACCA | | [2257-2277] 3′UTR | — |
| 48 | GCAUUUUGUCUCUUUGUAUCU | AGAUACAAAGAGACAAAAUGC | | [1984-2004] 3′UTR | [1644-1664] 3′UTR |
| 49 | GUAGUGGGAGUUUUCUUAUGU | ACAUAAGAAAACUCCCACUAC | | [1779-1799] ORF | [1496-1516] ORF |
| 50 | GCAUUAAACUUUGACAAGGCC | GGCCUUGUCAAAGUUUAAUGC | | [1662-1682] ORF | [1379-1399] ORF |
| 51 | UGAAAUCCAUCUGGUACAAAU | AUUUGUACCAGAUGGAUUUCA | | [1447-1467] ORF | [1311-1331] ORF |
| 52 | GCAUCCUUUUACUUUGACCUC | GAGGUCAAAGUAAAAGGAUGC | | [1217-1237] ORF | [1081-1101] ORF |
| 53 | GGCAUCCUUUUACUUUGACCU | AGGUCAAAGUAAAAGGAUGCC | | [1216-1236] ORF | [1080-1100] ORF |
| 54 | AGUUUUGGAAUUGCAGAUGAA | UUCAUCUGCAAUUCCAAAACU | | [1124-1144] ORF | [988-1008] ORF |
| 55 | GUGUGCAGAGUCUUUUGAGAU | AUCUCAAAAGACUCUGCACAC | | [932-952] ORF | [796-816] ORF |
| 56 | GGAGAGCAUGAAUGAGAGUCA | UGACUCUCAUUCAUGCUCUCC | | [902-922] ORF | [766-786] ORF |
| 57 | CGACAGUGGAGUAUGUGACAU | AUGUCACAUACUCCACUGUCG | | [694-714] ORF | [558-578] ORF |
| 58 | GAAAAAUCCUUGGGUCAACAU | AUGUUGACCCAAGGAUUUUUC | | [334-354] ORF | [198-218] ORF |
| 59 | CCGACAAAUACUACUACACAA | UUGUGUAGUAGUAUUUGUCGG | | [313-333] ORF | [177-197] ORF |
| 60 | GCCGACAAAUACUACUACACA | UGUGUAGUAGUAUUUGUCGGC | | [312-332] ORF | [176-196] ORF |
| 61 | UGCCUUCCUGAAAUAUGAGAA | UUCUCAUAUUUCAGGAAGGCA | | [290-310] ORF | [154-174] ORF |
| 62 | GCUGAAUGUUUUCCUGUUUGU | ACAAACAGGAAAACAUUCAGC | | [266-286] ORF | [130-150] ORF |
| 63 | CAGUUUUGUUUCUGGUUGUUU | AAACAACCAGAAACAAAACUG | | [238-258] ORF | [102-122] ORF |
| 64 | GUCACUUUUGAUAUGGUCUUA | UAAGACCAUAUCAAAAGUGAC | | [2498-2518] 3′UTR | — |
| 65 | GGUUCAGAGUUAUACAACUAA | UUAGUUGUAUAACUCUGAACC | | [2258-2278] 3′UTR | — |
| 66 | CCAGAGAAGGAAGGCGGAAUA | UAUUCCGCCUUCCUUCUCUGG | | [20-40] 5′UTR | — |
| 67 | UGAGCAGUCACUUUAGGAUAA | UUAUCCUAAAGUGACUGCUCA | | [2033-2053] 3′UTR | [1693-1713] 3′UTR |
| 68 | CUGCAUUUUGUCUCUUUGUAU | AUACAAAGAGACAAAAUGCAG | | [1982-2002] 3′UTR | [1642-1662] 3′UTR |
| 69 | GAAAGGUUCAAUUCUACUUCA | UGAAGUAGAAUUGAACCUUUC | | [1864-1884] ORF | [1581-1601] ORF |
| 70 | AGACCACAACCUCAAAACAAA | UUUGUUUUGAGGUUGUGGUCU | | [1478-1498] ORF | [1342-1362] ORF |
| 71 | CCCUUUGCUUCUAUCUUGAAA | UUUCAAGAUAGAAGCAAAGGG | | [1431-1451] ORF | [1295-1315] ORF |
| 72 | ACAGAAAAUCUCAUAAGGGCU | AGCCCUUAUGAGAUUUUCUGU | | [1290-1310] ORF | [1154-1174] ORF |
| 73 | GGACAGAAAAUCUCAUAAGGG | CCCUUAUGAGAUUUUCUGUCC | | [1288-1308] ORF | [1152-1172] ORF |
| 74 | GGUCAUUCUUUAUAUCUGUGA | UCACAGAUAUAAAGAAUGACC | | [1034-1054] ORF | [898-918] ORF |
| 75 | CGGAGGAGUUAUUUUGAAGUC | GACUUCAAAAUAACUCCUCCG | | [795-815] ORF | [659-679] ORF |
| 76 | CCGGAGGAGUUAUUUUGAAGU | ACUUCAAAAUAACUCCUCCGG | | [794-814] ORF | [658-678] ORF |
| 77 | CAGUGGAGUAUGUGACAUUCA | UGAAUGUCACAUACUCCACUG | | [697-717] ORF | [561-581] ORF |
| 78 | ACAGUGGAGUAUGUGACAUUC | GAAUGUCACAUACUCCACUGU | | [698-718] ORF | [560-580] ORF |
| 79 | ACACCUGUUUAACUUUGACUG | CAGUCAAAGUUAAACAGGUGU | | [560-580] ORF | [424-444] ORF |
| 80 | CACACCUGUUUAACUUUGACU | AGUCAAAGUUAAACAGGUGUG | | [559-579] ORF | [423-443] ORF |
| 81 | AGAAAGCAAUUGGAUCACAAC | GUUGUGAUCCAAUUGCUUUCU | | [477-497] ORF | [341-361] ORF |
| 82 | CGUCUGCUCUCUGCUUGAAUU | AAUUCAAGCAGAGAGCAGACG | | [370-390] ORF | [234-254] ORF |
| 83 | GGCCGACAAAUACUACUACAC | GUGUAGUAGUAUUUGUCGGCC | | [311-331] ORF | [175-195] ORF |
| 84 | GUCUGUAGUGGGAGUUUUCUU | AAGAAAACUCCCACUACAGAC | | [1775-1795] ORF | [1492-1512] ORF |
| 85 | GAAUUAGGCAAAGUGGGUUUU | AAAACCCACUUUGCCUAAUUC | | [1587-1607] ORF | — |
| 86 | UGCCUUUUCCUGGUUCAACAA | UUGUUGAACCAGGAAAAGGCA | | [1535-1555] ORF | — |
| 87 | UUGAAAUCCAUCUGGUACAAA | UUUGUACCAGAUGGAUUUCAA | | [1446-1466] ORF | [1310-1330] ORF |
| 88 | CCUUUUACUUUGACCUCUGCU | AGCAGAGGUCAAAGUAAAAGG | | [1221-1241] ORF | [1085-1105] ORF |
| 89 | CACCGGUCAUUCUUUAUAUCU | AGAUAUAAAGAAUGACCGGUG | | [1030-1050] ORF | [894-914] ORF |
| 90 | GCAGAGUCUUUUGAGAUGUGG | CCACAUCUCAAAAGACUCUGC | | [936-956] ORF | [800-820] ORF |
| 91 | GUGCAGAGUCUUUUGAGAUGU | ACAUCUCAAAAGACUCUGCAC | | [934-954] ORF | [798-818] ORF |
| 92 | AGGAGUUAUUUUGAAGUCUUC | GAAGACUUCAAAAUAACUCCU | | [798-818] ORF | [662-682] ORF |
| 93 | CUCCAGCCUAUCUCAUGAUGA | UCAUCAUGAGAUAGGCUGGAG | | [629-649] ORF | [493-513] ORF |
| 94 | CCAUUCUCUCCAGCCUAUCUC | GAGAUAGGCUGGAGAGAAUGG | | [622-642] ORF | [486-506] ORF |
| 95 | CCUACAUACAGCUAUUCACAU | AUGUGAAUAGCUGUAUGUAGG | | [533-553] ORF | [397-417] ORF |
| 96 | GCUCUCUGCUUGAAUUUUAAC | GUUAAAAUUGAAGCAGAGAGC | | [375-395] ORF | [239-259] ORF |
| 97 | CCCAGGUGAUUUUAUCAAUUC | GAAUUGAUAAAAUCACCUGGG | | [2280-2300] 3′UTR | — |
| 98 | AUACAACUAAUCCCAGGUGAU | AUCACCUGGGAUUAGUUGUAU | | [2269-2289] 3′UTR | — |
| 99 | UGAUUGCAUUCAACUUCGUUA | UAACGAAGUUGAAUGCAAUCA | | [2095-2115] 3′UTR | — |
| 100 | CAAGUCUGUAGUGGGAGUUUU | AAAACUCCCACUACAGACUUG | | [1772-1792] ORF | [1489-1509] ORF |
| 101 | GCAAUAUUGUUGGUCAUGCAG | CUGCAUGACCAACAAUAUUGC | | [1642-1662] ORF | [1367-1379] |
| | | | | | (13/13) ORF |
| 102 | GGAUUGCUGAAUCUUCCCUGU | ACAGGGAAGAUUCAGCAAUCC | | [154-174] 5′UTR | [18-38] 5′UTR |
| 103 | UGCAGACCACAACCUCAAAAC | GUUUUGAGGUUGUGGUCUGCA | | [1475-1495] ORF | [1339-1359] ORF |
| 104 | CUUGCACCGGUCAUUCUUUAU | AUAAAGAAUGACCGGUGCAAG | | [1026-1046] ORF | [890-810] ORF |
| 105 | GAGUCUUUUGAGAUGUGGGAU | AUCCCACAUCUCAAAAGACUC | | [939-959] ORF | [803-823] ORF |
| 106 | UGCAGAGUCUUUUGAGAUGUG | CACAUCUCAAAAGACUCUGCA | | [935-955] ORF | [799-819] ORF |
| 107 | CAAACAGAGGAGAGCAUGAAU | AUUCAUGCUCUCCUCUGUUUG | | [894-914] ORF | [758-778] ORF |
| 108 | UGGCUUAGGGAUUCACGGCAU | AUGCCGUGAAUCCCUAAGCCA | | [854-874] ORF | [718-738] ORF |
| 109 | CACCACCUUUUUAUCUUCUAU | AUAGAAGAUAAAAAGGUGGUG | | [828-848] ORF | [692-712] ORF |
| 110 | UCACCACCUUUUUAUGUUCUA | UAGAAGAUAAAAAGGUGGUGA | | [827-847] ORF | [691-711] ORF |
| 111 | UCACAUCUUAAAGGCUCACAG | CUGUGAGCCUUUAAGAUGUGA | | [84-104] 5′UTR | — |
| 112 | UCCGGAGGAGUUAUUUUGAAG | CUUCAAAAUAACUCCUCCGGA | | [793-813] ORF | [657-677] ORF |
| 113 | ACAAUAGCCUUGAUUCUCAUG | CAUGAGAAUCAAGGCUAUUGU | | [750-770] ORF | [614-634] ORF |
| 114 | CUACAUACAGCUAUUCACAUC | GAUGUGAAUAGCUGUAUGUAG | | [534-554] ORF | [398-418] ORF |
| 115 | UGAGAAAGCAAUUGGAUCACA | UGUGAUCCAAUUGCUUUCUCA | | [475-495] ORF | [339-359] ORF |
| 116 | ACACUGAGAAAGCAAUUGGAU | AUCCAAUUGCUUUCUCAGUGU | | [471-491] ORF | [335-355] ORF |
| 117 | AGAAAAAUCCUUGGGUCAACA | UGUUGACCCAAGGAUUUUUCU | | [333-353] ORF | [197-217] ORF |
| 118 | GACAAAUACUACUACACAAGA | UCUUGUGUAGUAGUAUUUGUC | | [315-335] ORF | [179-199] ORF |
| 119 | CUGAAUGUUUUCCUGUUUGUG | CACAAACAGGAAAACAUUCAG | | [267-287] ORF | [131-151] ORF |
| 120 | GGUUGUUUGGUUAGGGCUGAA | UUCAGCCCUAAGCAAACAACC | | [251-271] ORF | [115-135] ORF |
| 121 | UCCCAGGUGAUUUUAUCAAUU | AAUUGAUAAAAUCACCUGGGA | | [2279-2299] 3′UTR | — |
| 122 | UUGAUUGCAUUCAACUUCGUU | AACGAAGUUGAAUGCAAUCAA | | [2094-2114] 3′UTR | — |
| 123 | GUAAUUUACUUGGUCUCGUCA | UGACGAGACGAAGUAAAUUAC | | [2008-2028] 3′UTR | [1668-1688] 3′UTR |
| 124 | GAGUUUUCUUAUGUGGCCCUC | GAGGGCCACAUAAGAAAACUC | | [1786-1806] ORF | [1503-1523] ORF |
| 125 | CAAUGAGUUUUCUACAAUAGC | GCUAUUGUAGAAAACUCAUUG | | [1739-1759] ORF | [1456-1476] ORF |
| 126 | GACAGCAAUAUUGUUGGUCAU | AUGACCAACAAUAUUGCUGUC | | [1638-1658] ORF | — |
| 127 | GAGAUGGAGGAAUUAGGCAAA | UUUGGCUAAUUCCUCCAUCUC | Ms | [1578-1598] ORF | — |
| 128 | GGAGAUGGAGGAAUUAGGCAA | UUGCCUAAUUCCUCCAUCUCC | | [1577-1597] ORF | — |
| 129 | CAGACCACAACCUCAAAACAA | UUGUUUUGAGGUUGUGGUCUG | | [1477-1497] ORF | [1341-1361] ORF |
| 130 | CCAGGAUUGAAGUGGAUGGUC | GACCAUCCACUUCAAUCCUGG | | [1336-1356] ORF | [1200-1220] ORF |
| 131 | ACAACAAUAUUCACCAAUUCC | GGAAUUGGUGAAUAUUGUUGU | | [1318-1338] ORF | [1180-1200] ORF |
| 132 | CAAAGUUUUGGAAUUGCAGAU | AUCUGCAAUUCCAAAAGUUUG | | [1121-1141] ORF | [985-1005] ORF |
| 133 | UGUGAUUACCAAGGUUGUUAU | AUAACAACCUUGGUAAUCACA | | [1091-1111] ORF | [955-975] ORF |
| 134 | GAGGAGAGCAUGAAUGAGAGU | ACUCUCAUUCAUGCUCUCCUC | | [900-920] ORF | [764-784] ORF |
| 135 | AGAGGAGAGCAUGAAUGAGAG | CUCUCAUUCAUGCUCUCCUCU | | [899-919] ORF | [763-783] ORF |
| 136 | CACAUCUUAAAGGCUCACAGA | UCUGUGAGCCUUUAAGAUGUG | | [85-105] 5′UTR | — |
| 137 | UGAAGUCUUCUGGUAUACUCA | UGAGUAUACCAGAAGACUUCA | | [809-829] ORF | [673-693] ORF |
| 138 | ACGACAGUGGAGUAUGUGACA | UGUCACAUACUCCACUGUCGU | | [693-713] ORF | [557-577] ORF |
| 139 | CCGAAACACGACAGUGGAGUA | UACUCCACUGUCGUGUUUCGG | | [686-706] ORF | [550-570] ORF |
| 140 | CUCCAUUCUCUCCAGCCUAUC | GAUAGGCUGGAGAGAAUGGAG | | [620-640] ORF | [484-504] ORF |
| 141 | CCUAUAUGAUCUGCCUACAUA | UAUGUAGGCAGAUCAUAUAGG | | [520-540] ORF | [384-404] ORF |
| 142 | UCACAACCUCACCUUCCACAA | UUGUGGAAGGUGAGGUUGUGA | | [491-511] ORF | [355-375] ORF |
| 143 | GAAGGCCGACAAAUACUACUA | UAGUAGUAUUUGUCGGCCUUC | | [308-328] ORF | [172-192] ORF |
| 144 | AAAUAUGAGAAGGCCGACAAA | UUUGUCGGCCUUCUCAUAUUU | | [300-320] ORF | [164-184] ORF |
| 145 | CUGAAAUAUGAGAAGGCCGAC | GUCGGCCUUCUCAUAUUUCAG | | [297-317] ORF | [161-181] ORF |
| 146 | UGGUUAGGGCUGAAUGUUUUC | GAAAACAUUCAGCCCUAACCA | | [258-278] ORF | [122-142] ORF |
| 147 | GGUUUGGUGGUUCAGAGUUAU | AUAACUCUGAACCACCAAACC | | [2250-2270] 3′UTR | — |
| 148 | ACAAUGGGAAACUGGGUGGUU | AACCACCCAGUUUCCCAUUGU | | [204224] | [6888] |
| | | | | 5′UTR + ORF | 5′UTR + ORF |
| 149 | CAACUUCGUUACUUGAGCUUC | GAAGCUCAAGUAACGAAGUUG | | [2105-2125] 3′UTR | — |
| 150 | CCUCUUGACAAUGGGAAACUG | CAGUUUCCCAUUGUCAAGAGG | | [197-217] | [61-81] |
| | | | | 5′UTR + ORF | 5′UTR + ORF |
| 151 | GUAAUCUGCAUUUUGUCUCUU | AAGAGACAAAAUGCAGAUUAC | | [1977-1997] 3′UTR | [1637-1657] 3′UTR |
| 152 | CAACCUGUUGACUUCCCUGGA | UCCAGGGAAGUCAACAGGUUG | | [1553-1573] ORF | — |
| 153 | ACCACAACCUCAAAACAAAAA | UUUUUGUUUUGAGGUUGUGGU | | [1480-1500] ORF | [1344-1364] ORF |
| 154 | UCCUUUUACUUUGACCUCUGC | GCAGAGGUCAAAGUAAAAGGA | | [1220-1240] ORF | [1084-1104] ORF |
| 155 | AUCCUUUUACUUUGACCUCUG | CAGAGGUCAAAGUAAAAGGAU | | [1219-1239] ORF | [1083-1103] ORF |
| 156 | GUGAUUACCAAGGUUGUUAUG | CAUAACAACCUUGGUAAUCAC | | [1092-1112] ORF | [956-976] ORF |
| 157 | GCUUAGGGAUUCACGGCAUUG | CAAUGCCGUGAAUCCCUAAGC | | [856-876] ORF | [720-740] ORF |
| 158 | GUAUACUCACCACCUUUUUAU | AUAAAAAGGUGGUGAGUAUAC | | [821-841] ORF | [685-705] ORF |
| 159 | CCUCCAUUCUCUCCAGCCUAU | AUAGGCUGGAGAGAAUGGAGG | | [619-639] ORF | [483-503] ORF |
| 160 | GCACACUGAGAAAGCAAUUGG | CCAAUUGCUUUCUCAGUGUGC | | [469-489] ORF | [333-353] ORF |
| 161 | GCUUCCUGUGUGUCGCAAUCU | AGAUUGCGACACACAGGAAGC | | [410-430] ORF | [274-294] ORF |
| 162 | AGGAAGGCGGAAUAAACUUAU | AUAAGUUUAUUCCGCCUUCCU | | [27-47] 5′UTR | — |
| 163 | AGGGCUGAAUGUUUUCCUGUU | AACAGGAAAACAUUCAGCCCU | | [263-283] ORF | [127-147] ORF |
| 164 | CUGUCACUUUUGAUAUGGUCU | AGACCAUAUCAAAAGUGACAG | | [2496-2516] 3′UTR | — |
| 165 | UCAGAGUUAUACAACUAAUCC | GGAUUAGUUGUAUAACUCUGA | | [2261-2281] 3′UTR | — |
| 166 | GGUGGUUCAGAGUUAUACAAC | GUUGUAUAACUCUGAACCACC | | [2255-2275] 3′UTR | — |
| 167 | CCCUGGUAUUCUUUUUUUGAU | AUCAAAAAAAGAAUACCAGGG | | [2078-2098] 3′UTR | — |
| 168 | GACUCCCUGGUAUUCUUUUUU | AAAAAAGAAUACCAGGGAGUC | | [2074-2094] 3′UTR | — |
| 169 | AGUAAUUUACUUGGUCUCGUC | GACGAGACCAAGUAAAUUACU | | [2007-2027] 3′UTR | [1667-1687] 3′UTR |
| 170 | GUCUCUUUGUAUCUUCAGUAA | UUACUGAAGAUACAAAGAGAC | | [1991-2011] 3′UTR | [1651-1671] 3′UTR |
| 171 | GGACGGUAAUCUGCAUUUUGU | ACAAAAUGCAGAUUACCGUCC | | [1972-1992] 3′UTR | [1632-1652] 3′UTR |
| 172 | AUAAGGACGGUAAUCUGCAUU | AAUGCAGAUUACCGUCCUUAU | | [1968-1988] 3′UTR | [1628-1648] 3′UTR |
| 173 | UUGUUGGUCAUGCAGCAUUAA | UUAAUGCUGCAUGACCAACAA | | [1648-1668] ORF | [1367-1385] |
| | | | | | (19/19) ORF |
| 174 | AUAUUGUUGGUCAUGCAGCAU | AUGCUGCAUGACCAACAAUAU | | [1645-1665] ORF | [1367-1382] |
| | | | | | (16/16) ORF |
| 175 | GAUUGCUGAAUCUUCCCUGUU | AACAGGGAAGAUUCAGCAAUC | | [155-175] 5′UTR | [19-39] 5′UTR |
| 176 | GGAACAGGAGAUGGAGGAAUU | AAUUCCUCCAUCUCCUGUUCC | | [1571-1591] ORF | — |
| 177 | CAGUAUGAAGUGGCUGUGCUG | CAGCACAGCCACUUCAUACUG | | [1386-1406] ORF | [1250-1270] ORF |
| 178 | GGGUCAAACAGAGGAGAGCAU | AUGCUCUCCUCUGUUUGACCC | | [890-910] ORF | [754-774] ORF |
| 179 | CUUGAUUCUCAUGGUAACUUC | GAAGUUACCAUGAGAAUCAAG | GP, | [758-778] ORF | [622-642] ORF |
| | | | Rabbit | | |
| 180 | CUGAGAAAGCAAUUGGAUCAC | GUGAUCCAAUUGCUUUCUCAG | | [474-494] ORF | [338-358] ORF |
| 181 | CCAGUGUGAACAGCAAUUUAU | AUAAAUUGCUGUUCACACUGG | | [2520-2540] 3′UTR | — |
| 182 | CAGAGAAGGAAGGCGGAAUAA | UUAUUCCGCCUUCCUUCUCUG | | [21-41] 5′UTR | — |
| 183 | UCAAUUCCAGUGUUACCAUCU | AGAUGGUAACACUGGAAUUGA | | [2294-2314] 3′UTR | [1329-1339] |
| | | | | | (11/11) ORF |
| 184 | GAGUUAUACAACUAAUCCCAG | CUGGGAUUAGUUGUAUAACUC | | [2264-2284] 3′UTR | — |
| 185 | CAGCAACUUAAGAACUUCUGA | UCAGAAGUUCUUAAGUUGCUG | | [2125-2145] 3′UTR | — |
| 186 | CAAUGGGAAACUGGGUGGUUA | UAACCACCCAGUUUCCCAUUG | | [205-225] | [69-89] |
| | | | | 5′UTR + ORF | 5′UTR + ORF |
| 187 | UUACUUGGUCUCGUCAGGUUU | AAACCUGACGAGACCAAGUAA | | [2013-2033] 3′UTR | [1673-1693] 3′UTR |
| 188 | ACCUCUUGACAAUGGGAAACU | AGUUUCCCAUUGUCAAGAGGU | | [196-216] | [60-80] |
| | | | | 5′UTR + ORF | 5′UTR + ORF |
| 189 | CUUCAGUAAUUUACUUGGUCU | AGACCAAGUAAAUUACUGAAG | | [2003-2023] 3′UTR | [1663-1683] 3′UTR |
| 190 | GGUAAUCUGCAUUUUGUCUCU | AGAGACAAAAUGCAGAUUACC | | [1976-1996] 3′UTR | [1636-1656] 3′UTR |
| 191 | GACGGUAAUCUGCAUUUUGUC | GACAAAAUGCAGAUUACCGUC | | [1973-1993] 3′UTR | [1633-1653] 3′UTR |
| 192 | AGGACGGUAAUCUGCAUUUUG | CAAAAUGCAGAUUACCGUCCU | | [1971-1991] 3′UTR | [1631-1651] 3′UTR |
| 193 | AAGGACGGUAAUCUGCAUUUU | AAAAUGCAGAUUACCGUCCUU | | [1970-1990] 3′UTR | [1630-1650] 3′UTR |
| 194 | UAAGGACGGUAAUCUGCAUUU | AAAUGCAGAUUACCGUCCUUA | | [1969-1989] 3′UTR | [1629-1649] 3′UTR |
| 195 | AGGAAUAAGGACGGUAAUCUG | CAGAUUACCGUCCUUAUUCCU | | [1964-1984] 3′UTR | [1624-1644] 3′UTR |
| 196 | ACAGCAAUAUUGUUGGUCAUG | CAUGACCAACAAUAUUGCUGU | | [1639-1659] ORF | — |
| 197 | AGAUGGAGGAAUUAGGCAAAG | CUUUGCCUAAUUCCUCCAUCU | | [1579-1599] ORF | — |
| 198 | GCCUUUUCCUGGUUCAACAAC | GUUGUUGAACCAGGAAAAGGC | | [1538-1558] ORF | — |
| 199 | CACAACCUCAAAACAAAAAAG | CUUUUUUGUUUUGAGGUUGUG | | [1482-1502] ORF | [1346-1366] ORF |
| 200 | GUUGUUAUGCACCCAUCCAAA | UUUGGAUGGGUGCAUAACAAC | | [1104-1124] ORF | [968-988] ORF |
| 201 | ACUGGAGUGAUCAUGACAAUA | UAUUGUCAUGAUCACUCCAGU | | [735-755] ORF | [599-619] ORF |
| 202 | CGAAACACGACAGUGGAGUAU | AUACUCCACUGUCGUGUUUCG | | [687-707] ORF | [551-571] ORF |
| 203 | AGGCCGACAAAUACUACUACA | UGUAGUAGUAUUUGUCGGCCU | | [310-330] ORF | [174-194] ORF |
| 204 | UGAGAAGGCCGACAAAUACUA | UAGUAUUUGUCGGCCUUCUCA | | [305-325] ORF | [169-189] ORF |
| 205 | GAAGGAAGGCGGAAUAAACUU | AAGUUUAUUCCGCCUUCCUUC | | [25-45] 5′UTR | — |
| 206 | GUUGUUUGGUUAGGGCUGAAU | AUUCAGCCCUAACCAAACAAC | | [252-272] ORF | [116-136] ORF |
| 207 | CAGUGUGAACAGCAAUUUAUU | AAUAAAUUGCUGUUCACACUG | | [2521-2541] 3′UTR | — |
| 208 | CUAAGUAGGGUGACUUUUUAA | UUAAAAAGUCACCCUACUUAG | | [2367-2387] 3′UTR | — |
| 209 | GGUGAUUUUAUCAAUUCCAGU | ACUGGAAUUGAUAAAAUCACC | | [2284-2304] 3′UTR | — |
| 210 | UGGUGGUUCAGAGUUAUACAA | UUGUAUAACUCUGAACCACCA | | [2254-2274] 3′UTR | — |
| 211 | GUUUGGUGGUUCAGAGUUAUA | UAUAACUCUGAACCACCAAAC | | [2251-2271] 3′UTR | — |
| 212 | AGAGGUUUGGUGGUUCAGAGU | ACUCUGAACCACCAAACCUCU | | [2247-2267] 3′UTR | — |
| 213 | GAAGUUCUUAAAGCCCAUGGA | UCCAUGGGCUUUAAGAACUUC | | [2161-2181] 3′UTR | — |
| 214 | UCUGAAGUUCUUAAAGCCCAU | AUGGGCUUUAAGAACUUCAGA | | [2158-2178] 3′UTR | — |
| 215 | AGCAACUUAAGAACUUCUGAA | UUCAGAAGUUCUUAAGUUGCU | | [2126-2148] 3′UTR | — |
| 216 | UCAGCAACUUAAGAACUUCUG | CAGAAGUUCUUAAGUUGCUGA | | [2124-2144] 3′UTR | — |
| 217 | UCCCUGGUAUUCUUUUUUUGA | UCAAAAAAAGAAUACCAGGGA | | [2077-2097] 3′UTR | — |
| 218 | CUCCCUGGUAUUCUUUUUUUG | CAAAAAAAGAAUACCAGGGAG | | [2076-2096] 3′UTR | — |
| 219 | UGACUCCCUGGUAUUCUUUUU | AAAAAGAAUACCAGGGAGUCA | | [2073-2093] 3′UTR | — |
| 220 | CUUGACUCCCUGGUAUUCUUU | AAAGAAUACCAGGGAGUCAAG | | [2071-2091] 3′UTR | — |
| 221 | CCUUGACUCCCUGGUAUUCUU | AAGAAUACCAGGGAGUCAAGG | | [2070-2090] 3′UTR | — |
| 222 | UCAGGUUUGAGCAGUCACUUU | AAAGUGACUGCUCAAACCUGA | | [2026-2046] 3′UTR | [1686-1705] 3′UTR |
| 223 | GUCAGGUUUGAGCAGUCACUU | AAGUGACUGCUCAAACCUGAC | | [2025-2045] 3′UTR | [1685-1705] 3′UTR |
| 224 | UUAUAGGAAUAAGGACGGUAA | UUACCGUCCUUAUUCCUAUAA | | [1960-1980] 3′UTR | [1620-1640] 3′UTR |
| 225 | GUUAUAGGAAUAAGGACGGUA | UACCGUCCUUAUUCCUAUAAC | | [1959-1979] 3′UTR | [1619-1639] 3′UTR |
| 226 | AGAAAAUCUCAUAAGGGCUUU | AAAGCCCUUAUGAGAUUUUCU | | [1292-1312] ORF | [1156-1176] ORF |
| 227 | UCCAUUCCUGAAGGACCUCUC | GAGAGGUCCUUCAGGAAUGGA | | [125-145] 5′UTR | — |
| 228 | AUCUUCUAUAUCCUUGGCUUA | UAAGCCAAGGAUAUAGAAGAU | | [840-860] ORF | [704-724] ORF |
| 229 | GCCUAUCUCAUGAUGAGAAAA | UUUUCUCAUCAUGAGAUAGGC | | [634-654] ORF | [496-518] ORF |
| 230 | GCUGGUGGCCUAUAUGAUCUG | CAGAUCAUAUAGGCCACCAGC | | [512-532] ORF | [378-396] ORF |
| 231 | CACACUGAGAAAGCAAUUGGA | UCCAAUUGCUUUCUCAGUGUG | | [470-490] ORF | [334-354] ORF |
| 232 | GUCUGCUCUCUGCUUGAAUUU | AAAUUCAAGCAGAGAGCAGAC | | [371-391] ORF | [235-255] ORF |
| 233 | AGAGAAGGAAGGCGGAAUAAA | UUUAUUCCGCCUUCCUUCUCU | | [22-42] 5′UTR | — |
| 234 | UCCAGUGUGAACAGCAAUUUA | UAAAUUGCUGUUCACACUGGA | | [2519-2539] 3′UTR | — |
| 235 | CCUGAGUUUUGGUUUGUAAUC | GAUUACAAACCAAAACUCAGG | | [2315-2335] 3′UTR | — |
| 236 | UGUUACCAUCUCCUGAGUUUU | AAAACUCAGGAGAUGGUAACA | | [2304-2324] 3′UTR | — |
| 237 | GUGUUACCAUCUCCUGAGUUU | AAACUCAGGAGAUGGUAACAC | | [2303-2323] 3′UTR | — |
| 238 | AGUGUUACCAUCUCCUGAGUU | AACUCAGGAGAUGGUAACACU | | [2302-2322] 3′UTR | — |
| 239 | CAGUGUUACCAUCUCCUGAGU | ACUCAGGAGAUGGUAACACUG | | [2301-2321] 3′UTR | — |
| 240 | AUCCCAGGUGAUUUUAUCAAU | AUUGAUAAAAUCACCUGGGAU | | [2278-2298] 3′UTR | — |
| 241 | AGGUUUGGUGGUUCAGAGUUA | UAACUCUGAACCACCAAACCU | | [2249-2269] 3′UTR | — |
| 242 | GCAGAGGUUUGGUGGUUCAGA | UCUGAACCACCAAACCUCUGC | | [2245-2265] 3′UTR | — |
| 243 | ACUCCCUGGUAUUCUUUUUUU | AAAAAAAGAAUACCAGGGAGU | | [2075-2095] 3′UTR | — |
| 244 | GCCUUGACUCCCUGGUAUUCU | AGAAUACCAGGGAGUCAAGGC | | [2069-2089] 3′UTR | — |
| 245 | CUCGUCAGGUUUGAGCAGUCA | UGACUGCUCAAACCUGACGAG | | [2022-2042] 3′UTR | [1682-1702] 3′UTR |
| 246 | CGGUAAUCUGCAUUUUGUCUC | GAGACAAAAUGCAGAUUACCG | | [1975-1995] 3′UTR | [1835-1655] 3′UTR |
| 247 | GGCAAAGUGGGUUUUCUAAAC | GUUUAGAAAACCCACUUUGCC | | [1593-1613] ORF | — |
| 248 | GGGCUUUCGAACAACAAUAUU | AAUAUUGUUGUUCGAAAGCCC | | [1308-1326] ORF | [1170-1190] ORF |
| 249 | GGACAAAUGUUCCAUUCCUGA | UCAGGAAUGGAACAUUUGUCC | | [115-135] 5′UTR | — |
| 250 | CUCACCACCUUUUUAUCUUCU | AGAAGAUAAAAAGGUGGUGAG | | [826-846] ORF | [690-710] ORF |
| 251 | AGCUACUGAGUUCAUCCGGAG | CUCCGGAUGAACUCAGUAGCU | | [779-799] ORF | [643-663] ORF |
| 252 | UGCUCUCUGCUUGAAUUUUAA | UUAAAAUUCAAGCAGAGAGCA | | [374-394] ORF | [238-258] ORF |
| 253 | CUGCUCUCUGCUUGAAUUUUA | UAAAAUUCAAGCAGAGAGCAG | | [373-393] ORF | [237-257] ORF |
| 254 | GUACAUGUUUACUGUCACUUU | AAAGUGACAGUAAACAUGUAC | | [2485-2505] 3′UTR | — |
| 255 | AGAUUUCCUAAGUAGGGUGAC | GUCACCCUACUUAGGAAAUCU | | [2360-2380] 3′UTR | — |
| 256 | AGCAGAGGUUUGGUGGUUCAG | CUGAACCACCAAACCUCUGCU | | [2244-2264] 3′UTR | — |
| 257 | UGACUGUAGCAAGGCUUGAUA | UAUCAAGCCUUGCUACAGUCA | | [2224-2244] 3′UTR | — |
| 258 | GCCCAUGGAUCCUUUCUCAGA | UCUGAGAAAGGAUCCAUGGGC | | [2173-2193] 3′UTR | — |
| 259 | AAGUUCUUAAAGCCCAUGGAU | AUCCAUGGGCUUUAAGAACUU | | [2162-2182] 3′UTR | — |
| 260 | UGUCUCUUUGUAUCUUCAGUA | UACUGAAGAUACAAAGAGACA | | [1990-2010] 3′UTR | [1650-1670] 3′UTR |
| 261 | UCUGCAUUUUGUCUCUUUGUA | UACAAAGAGACAAAAUGCAGA | | [1961-2001] 3′UTR | [1641-1681] 3′UTR |
| 262 | GGGACAAUGAGUUUUCUACAA | UUGUAGAAAACUCAUUGUCCC | | [1735-1755] ORF | [1452-1472] ORF |
| 263 | UGGGACAGCAAUAUUGUUGGU | ACCAACAAUAUUGCUGUCCCA | | [1635-1655] ORF | — |
| 264 | UUAGGCAAAGUGGGUUUUCUA | UAGAAAACCCACUUUGCCUAA | | [1590-1610] ORF | — |
| 265 | GAACAGGAGAUGGAGGAAUUA | UAAUUCCUCCAUCUCCUGUUC | | [1572-1592] ORF | — |
| 266 | AGAUGUGGGAUGAUCGUGACU | AGUCACGAUCAUCCCACAUCU | | [949-969] ORF | [813-833] ORF |
| 267 | GAGAUGUGGGAUGAUCGUGAC | GUCACGAUCAUCCCACAUCUC | | [948-968] ORF | [812-832] ORF |
| 268 | GUUCAUCCGGAGGAGUUAUUU | AAAUAACUCCUCCGGAUGAAC | | [788-808] ORF | [652-672] ORF |
| 269 | GUGUCGCAAUCUGCUGUCCUU | AAGGACAGCAGAUUGCGACAC | | [419-439] ORF | [283-303] ORF |
| 270 | GAGGUUUGGUGGUUCAGAGUU | AACUCUGAACCACCAAACCUC | | [2248-2268] 3′UTR | — |
| 271 | AGCCCAUGGAUCCUUUCUCAG | CUGAGAAAGGAUCCAUGGGCU | | [2172-2192] 3′UTR | — |
| 272 | CUGAAGUUCUUAAAGCCCAUG | CAUGGGCUUUAAGAACUUCAG | | [2159-2179] 3′UTR | — |
| 273 | GCUUCAGCAACUUAAGAACUU | AAGUUCUUAAGUUGCUGAAGC | | [2121-2141] 3′UTR | — |
| 274 | UCGUCAGGUUUGAGCAGUCAC | GUGACUGCUCAAACCUGACGA | | [2023-2043] 3′UTR | [1683-1703] 3′UTR |
| 275 | AGGCAAAGUGGGUUUUCUAAA | UUUAGAAAACCCACUUUGCCU | | [1592-1612] ORF | — |
| 276 | CUUGAAAUCCAUCUGGUACAA | UUGUACCAGAUGGAUUUCAAG | | [1445-1465] ORF | [1309-1329] ORF |
| 277 | UGAACAAGCGUGGCUUCAGCA | UGCUGAAGCCACGCUUGUUCA | | [1141-1181] ORF | [1005-1025] ORF |
| 278 | CCAGCAGAAGGUUGUGAUUAC | GUAAUCACAACCUUCUGCUGG | | [1079-1099] ORF | [943-963] ORF |
| 279 | GCGCUGGACAAAUGUUCCAUU | AAUGGAACAUUUGUCCAGCGC | | [110-130] 5′UTR | — |
| 280 | AGCCUAUCUCAUGAUGAGAAA | UUUCUCAUCAUGAGAUAGGCU | | [633-653] ORF | [497-517] ORF |
| 281 | CUGAUCCUGCUUCCUGUGUGU | ACACACAGGAAGCAGGAUCAG | | [402-422] ORF | [266-286] ORF |
| 282 | GCAAGGCUUGAUAGCAGAGGU | ACCUCUGCUAUCAAGCCUUGC | | [2232-2252] 3′UTR | — |
| 283 | GGAUCCUUUCUCAGAAAAAUA | UAUUUUUCUGAGAAAGGAUCC | | [2179-2199] 3′UTR | — |
| 284 | GGGAGACCAAUGUGGGACAAU | AUUGUCCCACAUUGGUCUCCC | Rat, Ms | [1722-1742] ORF | [1439-1459] ORF |
| 285 | GACAUCGUGACAGGUCUGAAA | UUUCAGACCUGUCACGAUGUC | | [1686-1706] ORF | [1403-1423] ORF |
| 286 | CCAUCCAAAGUUUUGGAAUUG | CAAUUCCAAAACUUUGGAUGG | | [1116-1136] ORF | [980-1000] ORF |
| 287 | CAAUCUGCUGUCCUUCCUGAG | CUCAGGAAGGACAGCAGAUUG | | [425-445] ORF | [289-309] ORF |
| 288 | GCAAUCUGCUGUCCUUCCUGA | UCAGGAAGGACAGCAGAUUGC | | [424-444] ORF | [288-308] ORF |
| 289 | GGCGGAAUAAACUUAUUCAUU | AAUGAAUAAGUUUAUUCCGCC | | [32-52] 5′UTR | — |
| 290 | CCACAGAAGAUUUCCUAAGUA | UACUUAGGAAAUCUUCUGUGG | | [2353-2373] 3′UTR | — |
| 291 | GUUCUUAAAGUUCUGAAGUUC | GAACUUCAGAACUUUAAGAAC | | [2147-2167] 3′UTR | — |
| 292 | GAUAUUCCAGUCUGGAUCCUA | UAGGAUCCAGACUGGAAUAUC | | [1843-1863] ORF | [1560-1580] ORF |
| 293 | GCGCAAAUGCUGUCACCGAUA | UAUCGGUGACAGCAUUUGCGC | GP | [1826-1846] ORF | [1543-1563] ORF |
| 294 | GCAGCAUUAAACUUUGACAAG | CUUGUCAAAGUUUAAUGCUGC | | [1659-1679] ORF | [1376-1396] ORF |
| 295 | GAAAAUCUCAUAAGGGCUUUC | GAAAGCCCUUAUGAGAUUUUC | | [1293-1313] ORF | [1157-1177] ORF |
| 296 | CGCUGGACAAAUGUUCCAUUC | GAAUGGAACAUUUGUCCAGCG | | [111-131] 5′UTR | — |
| 297 | GAGUUCAUCCGGAGGAGUUAU | AUAACUCCUCCGGAUGAACUC | | [786-806] ORF | [650-670] ORF |
| 298 | CUAUCUCAUGAUGAGAAAAAG | CUUUUUCUCAUCAUGAGAUAG | | [636-656] ORF | [500-520] ORF |
| 299 | CCUAUCUCAUGAUGAGAAAAA | UUUUUCUCAUCAUGAGAUAGG | | [635-655] ORF | [499-519] ORF |
| 300 | CCUGUUUAACUUUGACUGCUA | UAGCAGUCAAAGUUAAACAGG | | [563-583] ORF | [427-447] ORF |
| 301 | GAAUUUUAACAGCACGCUGAU | AUCAGCGUGCUGUUAAAAUUC | | [386-406] ORF | [250-270] ORF |
| 302 | CCACUGGUUUUCAGUUUUGUU | AACAAAACUGAAAACCAGUGG | | [227-247] ORF | [91-111] ORF |
| 303 | UGGGACAAUGAGUUUUCUACA | UGUAGAAAACUCAUUGUCCCA | | [1734-1754] ORF | [1451-1471] ORF |
| 304 | UGUGGGACAAUGAGUUUUCUA | UAGAAAACUCAUUGUCCCACA | Rat, Ms | [1732-1752] ORF | [1449-1469] ORF |
| 305 | AUAAGGGCUUUCGAACAACAA | UUGUUGUUCGAAAGCCCUUAU | | [1302-1322] ORF | [1166-1186] ORF |
| 306 | UCAUAAGGGCUUUCGAACAAC | GUUGUUCGAAAGCCCUUAUGA | | [1300-1320] ORF | [1164-1184] ORF |
| 307 | CAGAAAAUCUCAUAAGGGCUU | AAGCCCUUAUGAGAUUUUCUG | | [1291-1311] ORF | [1155-1175] ORF |
| 308 | GAGGAAGAUUUCUUCUCCAUU | AAUGGAGAAGAAAUCUUCCUC | | [1245-1265] ORF | [1109-1129] ORF |
| 309 | UGGAUCCUUGCACCGGUCAUU | AAUGACCGGUGCAAGGAUCCA | | [1020-1040] ORF | [884-904] ORF |
| 310 | UGAUCGUGACUCCCACUGUAG | CUACAGUGGGAGUCACGAUCA | | [959-979] ORF | [823-843] ORF |
| 311 | GUGGAUGCCUUCCUGAAAUAU | AUAUUUCAGGAAGGCAUCCAC | | [285-305] ORF | [149-169] ORF |
| 312 | UGAGUUUUCUACAAUAGCUAC | GUAGCUAUUGUAGAAAACUCA | | [1742-1762] ORF | [1459-1479] ORF |
| 313 | CGGAUGGGACAGCAAUAUUGU | ACAAUAUUGCUGUCCCAUCCG | | [1631-1651] ORF | — |
| 314 | CUGGAACAGGAGAUGGAGGAA | UUCCUCCAUCUCCUGUUCCAG | | [1569-1589] ORF | — |
| 315 | AGUGAGGAUGUUUUCCAGUAU | AUACUGGAAAACAUCCUCACU | Ms | [1371-1391] ORF | [1235-1255] ORF |
| 316 | CUCAUAAGGGCUUUCGAACAA | UUGUUCGAAAGCCCUUAUGAG | | [1299-1319] ORF | [1163-1183] ORF |
| 317 | GGAAGAUUUCUUCUCCAUUCA | UGAAUGGAGAAGAAAUCUUCC | | [1247-1267] ORF | [1111-1131] ORF |
| 318 | CCCAUCCAAAGUUUUGGAAUU | AAUUCCAAAACUUUGGAUGGG | | [1115-1135] ORF | [979-999] ORF |
| 319 | CCAAGGUUGUUAUGCACCCAU | AUGGGUGCAUAACAACCUUGG | | [1099-1119] ORF | [963-983] ORF |
| 320 | CGCCCUAAGUUUGAAGGGCAU | AUGCCCUUCAAACUUAGGGCG | | [981-1001] ORF | [845-865] ORF |
| 321 | GGUGUGUGUUUUUCACAUCUU | AAGAUGUGAAAAACACACACC | | [72-92] 5′UTR | — |
| 322 | CUGUUUAACUUUGACUGCUAU | AUAGCAGUCAAAGUUAAACAG | | [564-584] ORF | [428-448] ORF |
| 323 | CCUAAGUAGGGUGACUUUUUA | UAAAAAGUCACCCUACUUAGG | | [2366-2386] 3′UTR | — |
| 324 | CCAUCUCCUGAGUUUUGGUUU | AAACCAAAACUCAGGAGAUGG | | [2309-2329] 3′UTR | — |
| 325 | GACUGUAGCAAGGCUUGAUAG | CUAUCAAGCCUUGCUACAGUC | | [2225-2245] 3′UTR | — |
| 326 | GCAUUCAACUUCGUUACUUGA | UCAAGUAACGAAGUUGAAUGC | | [2100-2120] 3′UTR | — |
| 327 | AGGUUUGAGCAGUCACUUUAG | CUAAAGUGACUGCUCAAACCU | | [2028-2048] 3′UTR | [1688-1708] 3′UTR |
| 328 | UGCAGCAUUAAACUUUGACAA | UUGUCAAAGUUUAAUGCUGCA | GP | [1658-1678] ORF | [1375-1395] ORF |
| 329 | CGAACAACAAUAUUCACCAAU | AUUGGUGAAUAUUGUUGUUCG | | [1313-1333] ORF | [1177-1197] ORF |
| 330 | GACUGGACAGAAAAUCUCAUA | UAUGAGAUUUUCUGUCCAGUC | Rat | [1284-1304] ORF | [1148-1168] ORF |
| 331 | GCAGAAGGUUGUGAUUACCAA | UUGGUAAUCACAACCUUCUGC | | [1062-1102] ORF | [946-968] ORF |
| 332 | UCUUUUGAGAUGUGGGAUGAU | AUCAUCCCACAUCUCAAAAGA | | [942-962] ORF | [806-826] ORF |
| 333 | GUCUUUUGAGAUGUGGGAUGA | UCAUCCCACAUCUCAAAAGAC | | [941-961] ORF | [805-825] ORF |
| 334 | GUGUGUGUUUUUCACAUCUUA | UAAGAUGUGAAAAACACACAC | | [73-93] 5′UTR | — |
| 335 | GCAAUUGGAUCACAACCUCAC | GUGAGGUUGUGAUCCAAUUGC | | [482-502] ORF | [346-366] ORF |
| 336 | UGUGGAUGCCUUCCUGAAAUA | UAUUUCAGGAAGGCAUCCACA | | [284-304] ORF | [148-168] ORF |
| 337 | UCCUAAGUAGGGUGACUUUUU | AAAAAGUCACCCUACUUAGGA | | [2365-2385] 3′UTR | — |
| 338 | CAGGUUUGAGCAGUCACUUUA | UAAAGUGACUGCUCAAACCUG | | [2027-2047] 3′UTR | [1687-1707] |
| | | | | | 3′UTR |
| 339 | GGAUGGGACAGCAAUAUUGUU | AACAAUAUUGCUGUCCCAUCC | | [1632-1652] ORF | — |
| 340 | GCACAGCCAGUGAGGAUGUUU | AAACAUCCUCACUGGCUGUGC | | [1363-1383] ORF | [1227-1247] ORF |
| 341 | AGGGCUUUCGAACAACAAUAU | AUAUUGUUGUUCGAAAGCCCU | | [1305-1325] ORF | [1169-1189] ORF |
| 342 | GAAGAUUUCUUCUCCAUUCAU | AUGAAUGGAGAAGAAAUCUUC | | [1248-1268] ORF | [1112-1132] ORF |
| 343 | AGAGGAAGAUUUCUUCUCCAU | AUGGAGAAGAAAUCUUCCUCU | | [1244-1264] ORF | [1108-1128] ORF |
| 344 | GGAGUAUGUGACAUUCACCAG | CUGGUGAAUGUCACAUACUCC | | [701-721] ORF | [565-585] ORF |
| 345 | AGGCGGAAUAAACUUAUUCAU | AUGAAUAAGUUUAUUCCGCCU | | [31-51] 5′UTR | — |
| 346 | UUGUUUCUGGUUGUUUGGUUA | UAACCAAACAACCAGAAACAA | | [243-263] ORF | [107-127] ORF |
| 347 | CACAGAAGAUUUCCUAAGUAG | CUACUUAGGAAAUCUUCUGUG | | [2354-2374] 3′UTR | — |
| 348 | CCCACAGAAGAUUUCCUAAGU | ACUUAGGAAAUCUUCUGUGGG | | [2352-2372] 3′UTR | — |
| 349 | CUCCUGAGUUUUGGUUUGUAA | UUACAAACCAAAACUCAGGAG | | [2313-2333] 3′UTR | — |
| 350 | GGGUGGUUAACCACUGGUUUU | AAAACCAGUGGUUAACCACCC | | [217-237] ORF | [81-101] ORF |
| 351 | UGGGUGGUUAACCACUGGUUU | AAACCAGUGGUUAACCACCCA | | [216-236] ORF | [80-100] ORF |
| 352 | UCUGAAGUUCUUAAAGUUCUG | CAGAACUUUAAGAACUUCAGA | | [2141-2161] 3′UTR | — |
| 353 | GGAUAAGAAUGUGCCUCUCAA | UUGAGAGGCACAUUCUUAUCC | | [2048-2068] 3′UTR | [1708-1728] |
| | | | | | 3′UTR |
| 354 | UCACUUUAGGAUAAGAAUGUG | CACAUUCUUAUCCUAAAGUGA | | [2040-2060] 3′UTR | [1700-1720] 3′UTR |
| 355 | CCCAAGUCUGUAGUGGGAGUU | AACUCCCACUACAGACUUGGG | | [1770-1790] ORF | [1487-1507] ORF |
| 356 | GUGGGUUUUCUAAACUACCGU | ACGGUAGUUUAGAAAACCCAC | | [1599-1619] ORF | — |
| 357 | ACUGGACAGAAAAUCUCAUAA | UUAUGAGAUUUUCUGUCCAGU | Rat | [1285-1305] ORF | [1149-1169] ORF |
| 358 | ACCCAUCCAAAGUUUUGGAAU | AUUCCAAAACUUUGGAUGGGU | | [1114-1134] ORF | [978-998] ORF |
| 359 | GCUGGACAAAUGUUCCAUUCC | GGAAUGGAACAUUUGUCCAGC | | [112-132] 5′UTR | — |
| 360 | GCAAGUGUGCAGAGUCUUUUG | CAAAAGACUCUGCACACUUGC | | [928-948] ORF | [792-812] ORF |
| 361 | GGAUUCACGGCAUUGGUGGAA | UUCCACCAAUGCCGUGAAUCC | | [862-882] ORF | [726-746] ORF |
| 362 | CCAGCCUAUCUCAUGAUGAGA | UCUCAUCAUGAGAUAGGCUGG | | [631-651] ORF | [495-515] ORF |
| 363 | UAACCACUGGUUUUCAGUUUU | AAAACUGAAAACCAGUGGUUA | | [224-244] ORF | [88-108] ORF |
| 364 | CAACUAAUCCCAGGUGAUUUU | AAAAUCACCUGGGAUUAGUUG | | [2272-2292] 3′UTR | — |
| 365 | CUGGGUGGUUAACCACUGGUU | AACCAGUGGUUAACCACCCAG | | [215-235] ORF | [79-99] ORF |
| 366 | CCAUGGAUCCUUUCUCAGAAA | UUUCUGAGAAAGGAUCCAUGG | | [2175-2195] 3′UTR | — |
| 367 | GAAGUUCUUAAAGUUCUGAAG | CUUCAGAACUUUAAGAACUUC | | [2144-2164] 3′UTR | — |
| 368 | CUGAAGUUCUUAAAGUUCUGA | UCAGAACUUUAAGAACUUCAG | | [2142-2162] 3′UTR | — |
| 369 | GGUUCAAUUCUACUUCAACAA | UUGUUGAAGUAGAAUUGAACC | Rat | [1868-1888] ORF | [1585-1605] ORF |
| 370 | CCAAUGUGGGACAAUGAGUUU | AAACUCAUUGUCCCACAUUGG | Rat, Ms | [1728-1748] ORF | [1445-1465] ORF |
| 371 | GGUCAUGCAGCAUUAAACUUU | AAAGUUUAAUGCUGCAUGACC | GP, Rat, | [1653-1673] ORF | [1370-1390] ORF |
| | | | Ms | | |
| 372 | CCGGAUGGGACAGCAAUAUUG | CAAUAUUGCUGUCCCAUCCGG | | [1630-1650] ORF | — |
| 373 | UGCUUCUAUCUUGAAAUGCAU | AUGGAUUUCAAGAUAGAAGCA | | [1436-1458] ORF | [1300-1320] ORF |
| 374 | CCCUCAAUCUCUCUCCUGGAA | UUCCAGGAGAGAGAUUGAGGG | | [1194-1214] ORF | [1058-1078] ORF |
| 375 | GCAGAUGAACAAGCGUGGCUU | AAGCCACGCUUGUUCAUCUGC | | [1136-1156] ORF | [1000-1020] ORF |
| 376 | GGAAUUGCAGAUGAACAAGCG | CGCUUGUUCAUCUGCAAUUCC | | [1130-1150] ORF | [994-1014] ORF |
| 377 | GACAAAUGUUCCAUUCCUGAA | UUCAGGAAUGGAACAUUUGUC | | [116-136] 5′UTR | — |
| 378 | UGGACAAAUGUUCGAUUCCUG | CAGGAAUGGAACAUUUGUCCA | | [114-134] 5′UTR | — |
| 379 | GCCCUAAGUUUGAAGGGCAUC | GAUGCCCUUCAAACUUAGGGC | | [982-1002] ORF | [846-866] ORF |
| 380 | GUAUGUGACAUUCACCAGCGU | ACGCUGGUGAAUGUCACAUAC | | [704-724] ORF | [566-586] (19/19) |
| | | | | | ORF |
| 381 | ACAGCUAUUCACAUCAUUGCA | UGCAAUGAUGUGAAUAGCUGU | | [540-560] ORF | [404-424] ORF |
| 382 | GCCUACAUACAGCUAUUCACA | UGUGAAUAGCUGUAUGUAGGC | | [532-552] ORF | [396-416] ORF |
| 383 | CUGCCUACAUACAGCUAUUCA | UGAAUAGCUGUAUGUAGGCAG | | [530-550] ORF | [394-414] ORF |
| 384 | CCGCACACUGAGAAAGCAAUU | AAUUGCUUUCUCAGUGUGCGG | | [467-487] ORF | [331-351] ORF |
| 385 | CUCAUUUUGCAGCCGCACACU | AGUGUGCGGCUGCAAAAUGAG | | [455-475] ORF | [319-339] ORF |
| 386 | UCAGUUUUGUUUCUGGUUGUU | AACAACCAGAAACAAAACUGA | | [237-257] ORF | [101-121] ORF |
| 387 | CCCAUAUAACACCAACAGUGU | ACACUGUUGGUGUUAUAUGGG | | [2466-2486] 3′UTR | — |
| 388 | GAAGAUUUCCUAAGUAGGGUG | CACCCUACUUAGGAAAUCUUC | | [2358-2378] 3′UTR | — |
| 389 | CCAUGACUGUAGCAAGGCUUG | CAAGCCUUGCUACAGUCAUGG | | [2221-2241] 3′UTR | — |
| 390 | UCAACUUCGUUACUUGAGCUU | AAGCUCAAGUAACGAAGUUGA | | [2104-2124] 3′UTR | — |
| 391 | AGAAUGUGCCUCUCAAGCCUU | AAGGCUUGAGAGGCACAUUCU | | [2053-2073] 3′UTR | [1713-1733] |
| | | | | | 3′UTR |
| 392 | CCACCUCUUGACAAUGGGAAA | UUUCCCAUUGUCAAGAGGUGG | | [194-214] | [58-78] |
| | | | | 5′UTR + ORF | 5′UTR + ORF |
| 393 | UGGAUCCUAGAAAGGUUCAAU | AUUGAACCUUUCUAGGAUCCA | | [1855-1875] ORF | [1572-1592] ORF |
| 394 | GUCAUGCAGCAUUAAACUUUG | CAAAGUUUAAUGCUGCAUGAC | GP, Rat, | [1654-1674] ORF | [1371-1391] ORF |
| | | | Ms | | |
| 395 | UGGGUUUUCUAAACUACCGUC | GACGGUAGUUUAGAAAACCCA | | [1600-1620] ORF | — |
| 396 | CCUGUUGACUUCCCUGGAACA | UGUUCCAGGGAAGUCAACAGG | | [1556-1576] ORF | — |
| 397 | GGUUCAACAACCUGUUGACUU | AAGUCAACAGGUUGUUGAACC | | [1546-1566] ORF | — |
| 398 | GAAUCCGGAUUGCUGAAUCUU | AAGAUUCAGCAAUGCGGAUUC | | [148-168] 5′UTR | [12-32] 5′UTR |
| 399 | CCUUUGCUUCUAUCUUGAAAU | AUUUCAAGAUAGAAGCAAAGG | | [1432-1452] ORF | [1296-1316] ORF |
| 400 | CCUGAAGGACCUCUCCAGAAU | AUUCUGGAGAGGUCCUUCAGG | | [131-151] 5′UTR | [1-15] (15/15) |
| | | | | | 5′UTR |
| 401 | GAACAAGCGUGGCUUCAGCAU | AUGCUGAAGCCACGCUUGUUC | | [1142-1162] ORF | [1006-1026] ORF |
| 402 | GAAUUGCAGAUGAACAAGCGU | ACGCUUGUUCAUCUGCAAUUC | | [1131-1151] ORF | [995-1015] ORF |
| 403 | GAAGGUUGUGAUUACCAAGGU | ACCUUGGUAAUGACAACCUUC | | [1085-1105] ORF | [949-969] ORF |
| 404 | CGCAAGUGUGCAGAGUCUUUU | AAAAGACUCUGCACACUUGCG | | [927-947] ORF | [791-811] ORF |
| 405 | AUAGCCUUGAUUCUCAUGGUA | UACCAUGAGAAUCAAGGCUAU | Rabbit | [753-773] ORF | [617-637] ORF |
| 406 | CUCUCCAGCCUAUCUCAUGAU | AUCAUGAGAUAGGCUGGAGAG | | [627-647] ORF | [491-511] ORF |
| 407 | UCACAUCAUUGCACACCUGUU | AACAGGUGUGCAAUGAUGUGA | | [548-568] ORF | [412-432] ORF |
| 408 | AGCUAUUCACAUCAUUGCACA | UGUGCAAUGAUGUGAAUAGCU | | [542-562] ORF | [406-426] ORF |
| 409 | GUGGCCUAUAUGAUCUGCCUA | UAGGCAGAUCAUAUAGGCCAC | | [516-536] ORF | [380-400] ORF |
| 410 | GGAUGCCUUCCUGAAAUAUGA | UCAUAUUUCAGGAAGGCAUCC | | [287-307] ORF | [151-171] ORF |
| 411 | GUGUACAUGUUUACUGUCACU | AGUGACAGUAAACAUGUACAC | | [2483-2503] 3′UTR | — |
| 412 | CCAUAUAACACCAACAGUGUA | UACACUGUUGGUGUUAUAUGG | | [2467-2487] 3′UTR | — |
| 413 | CAAGGCUUGAUACCAGAGGUU | AACCUCUGCUAUCAAGCCUUG | | [2233-2253] 3′UTR | — |
| 414 | GCCAUGACUGUAGCAAGGCUU | AAGCCUUGCUACAGUCAUGGC | | [2220-2240] 3′UTR | — |
| 415 | AUGGAUCCUUCCUCAGAAAAA | UUUUUCUGAGAAAGGAUCCAU | | [2177-2197] 3′UTR | — |
| 416 | CAUGGAUCCUUUCUCAGAAAA | UUUUCUGAGAAAGGAUCCAUG | | [2176-2196] 3′UTR | — |
| 417 | GAUAAGAAUGUGCCUCUCAAG | CUUGAGAGGCACAUUCUUAUC | | [2049-2069] 3′UTR | [1709-1729] 3′UTR |
| 418 | AGGAUAAGAAUGUGCCUCUCA | UGAGAGGCACAUUCUUAUCCU | | [2047-2067] 3′UTR | [1707-1727] 3′UTR |
| 419 | GGAUCCUAGAAAGGUUCAAUU | AAUUGAACCUUUCUAGGAUCC | | [1856-1876] ORF | [1573-1593] ORF |
| 420 | GAGACCAAUGUGGGACAAUGA | UCAUUGUCCCACAUUGGUCUC | Rat, Ms | [1724-1744] ORF | [1441-1461] ORF |
| 421 | GUGACAGGUCUGAAACAGAAA | UUUCUGUUUCAGACCUGUCAC | | [1692-1712] ORF | [1409-1429] ORF |
| 422 | GGGUUUUCUAAACUACCGUCU | AGACGGUAGUUUAGAAAACCC | | [1601-1621] ORF | — |
| 423 | UGGUUCAACAACCUGUUGACU | AGUCAACAGGUUGUUGAACCA | | [1545-1565] ORF | — |
| 424 | UGGUACAAAUUCCAGUGUGCA | UGCACACUGGAAUUUGUACCA | | [1458-1478] ORF | [1322-1342] ORF |
| 425 | CCAUCUGGUACAAAUUCCAGU | ACUGGAAUUUGUACCAGAUGG | | [1453-1473] ORF | [1317-1337] ORF |
| 426 | CCAGAAUCCGGAUUGCUGAAU | AUUCAGCAAUCCGGAUUCUGG | | [145-165] 5′UTR | [9-29] 5′UTR |
| 427 | ACCUCUCCAGAAUCCGGAUUG | CAAUCCGGAUUCUGGAGAGGU | | [139-159] 5′UTR | [3-23] 5′UTR |
| 428 | UCGAACAACAAUAUUCACCAA | UUGGUGAAUAUUGUUGUUCGA | | [1312-1332] ORF | [1176-1196] ORF |
| 429 | GCUUUCGAACAACAAUAUUCA | UGAAUAUUGUUGUUCGAAAGC | | [1308-1328] ORF | [1172-1192] ORF |
| 430 | UCCAUUCAUAUCCGAGCAGCA | UGCUGCUCGGAUAUGAAUGGA | | [1260-1280] ORF | [1124-1144] ORF |
| 431 | CCAGAGGAAGAUUUCUUCUCC | GGAGAAGAAAUCUUCCUCUGG | | [1242-1262] ORF | [1106-1126] ORF |
| 432 | CUGGAAUGGCAUCCUUUUACU | AGUAAAAGGAUGCCAUUCCAG | | [1209-1229] ORF | [1073-1093] ORF |
| 433 | CUCUCCUGGAAUGGCAUCCUU | AAGGAUGCCAUUCCAGGAGAG | | [1204-1224] ORF | [1068-1088] ORF |
| 434 | UUAUGCACCCAUCCAAAGUUU | AAACUUUGGAUGGGUGCAUAA | | [1108-1128] ORF | [972-992] ORF |
| 435 | AAGGUUGUGAUUACCAAGGUU | AACCUUGGUAAUCACAACCUU | | [1086-1106] ORF | [950-970] ORF |
| 436 | CAGAAGGUUGUGAUUACCAAG | CUUGGUAAUCACAACCUUCUG | | [1083-1103] ORF | [947-967] ORF |
| 437 | GUUUUACCGCUCCGAGCAGAA | UUCUGCUGGGAGCGGUAAAAC | | [1067-1087] ORF | [931-951] ORF |
| 438 | UGCGCUGGACAAAUGUUCCAU | AUGGAACAUUUGUCCAGCGCA | | [109-129] 5′UTR | — |
| 439 | GCAUGAAUGAGAGUCAUCCUC | GAGGAUGACUCUCAUUCAUGC | | [907-927] ORF | [771-791] ORF |
| 440 | UCUGGUAUACUCACCACCUUU | AAAGGUGGUGAGUAUACCAGA | | [817-837] ORF | [681-701] ORF |
| 441 | UCAUGGUAACUUCAGGUACUG | CAGUAGCUGAAGUUACCAUGA | | [766-786] ORF | [630-650] ORF |
| 442 | GCCUUGAUUCUCAUGGUAACU | AGUUACCAUGAGAAUCAAGGC | GP, | [756-776] ORF | [620-640] ORF |
| | | | Rabbit | | |
| 443 | UAGCCUUGAUUCUCAUGGUAA | UUACCAUGAGAAUCAAGGCUA | GP, | [754-774] ORF | [618-638] ORF |
| | | | Rabbit | | |
| 444 | GACAAUAGCCUUGAUUCUCAU | AUGAGAAUCAAGGCUAUUGUC | | [749-769] ORF | [613-633] ORF |
| 445 | GAAACACGACAGUGGAGUAUG | CAUACUCCACUGUCGUGUUUC | | [668-708] ORF | [552-572] ORF |
| 446 | AGGUGUGUGUUUUUCACAUCU | AGAUGUGAAAAACACACACCU | | [71-91] 5′UTR | — |
| 447 | UGAUCUGCCUACAUACAGCUA | UAGCUGUAUGUAGGCAGAUCA | | [526-546] ORF | [390-410] ORF |
| 448 | CCACAAGCUGGUGGCCUAUAU | AUAUAGGCCACCAGCUUGUGG | | [506-526] ORF | [370-390] ORF |
| 449 | CGGAAUAAACUUAUUCAUUCC | GGAAUGAAUAAGUUUAUUCCG | | [34-54] 5′UTR | — |
| 450 | ACCACUGGUUUUCAGUUUUGU | ACAAAACUGAAAACCAGUGGU | | [226-246] ORF | [90-110] ORF |
| 451 | UGUACAUGUUUACUGUCACUU | AAGUGACAGUAAACAUGUAGA | | [2484-2504] 3′UTR | — |
| 452 | UCUCCUGAGUUUUGGUUUGUA | UACAAACCAAAACUCAGGAGA | | [2312-2332] 3′UTR | — |
| 453 | UAAAUCUUUCUGGACAGCCAU | AUGGCUGUCCAGAAAGAUUUA | | [2204-2224] 3′UTR | — |
| 454 | UGGAUCCUUUCUCAGAAAAAU | AUUUUUCUGAGAAAGGAUCCA | | [2178-2198] 3′UTR | — |
| 455 | CGAUAUUCCAGUCUGGAUCCU | AGGAUCCAGACUGGAAUAUCG | | [1842-1862] ORF | [1559-1579] ORF |
| 456 | CCGAUAUUCCAGUCUGGAUCC | GGAUCCAGACUGGAAUAUCGG | | [1841-1861] ORF | [1558-1578] ORF |
| 457 | CGCAAAUGCUGUCACCGAUAU | AUAUCGGUGACAGCAUUUGCG | GP | [1827-1847] ORF | [1544-1564] ORF |
| 458 | UGCGCAAAUGCUGUCACCGAU | AUCGGUGACAGCAUUUGCGCA | GP | [1825-1845] ORF | [1542-1562] ORF |
| 459 | UCAUGCAGCAUUAAACUUUGA | UCAAAGUUUAAUGCUGCAUGA | GP, Rat, | [1655-1675] ORF | [1372-1392] ORF |
| | | | Ms | | |
| 460 | GACAGGUGCCUUUUCCUGGUU | AACCAGGAAAAGGCACCUGUC | GP | [1529-1549] ORF | — |
| 461 | CUGGUACAAAUUCCAGUGUGC | GCACACUGGAAUUUGUACCAG | | [1457-1477] ORF | [1321-1341] ORF |
| 462 | AGAAUCCGGAUUGCUGAAUCU | AGAUUCAGCAAUCCGGAUUCU | | [147-167] 5′UTR | [11-31] 5′UTR |
| 463 | CUCUCCAGAAUCCGGAUUGCU | AGCAAUCCGGAUUCUGGAGAG | | [141-161] 5′UTR | [5-25] 5′UTR |
| 464 | GGCUUUCGAACAACAAUAUUC | GAAUAUUGUUGUUCGAAAGCC | | [1307-1327] ORF | [1171-1191] ORF |
| 465 | CUGAAGGACCUCUCCAGAAUC | GAUUCUGGAGAGGUCCUUCAG | | [132-152] 5′UTR | [1-16] (16/16) |
| | | | | | 5′UTR |
| 466 | GGACUGGACAGAAAAUCUCAU | AUGAGAUUUUCUGUCCAGUCC | Rat | [1283-1303] ORF | [1147-1167] ORF |
| 467 | GGAAUGGCAUCCUUUUACUUU | AAAGUAAAAGGAUGCCAUUCC | | [1211-1231] ORF | [1075-1095] ORF |
| 468 | UCCUGGAAUGGCAUCCUUUUA | UAAAAGGAUGCCAUUCCAGGA | | [1207-1227] ORF | [1071-1091] ORF |
| 469 | CAAGGUUGUUAUGCACCCAUC | GAUGGGUGCAUAACAACCUUG | | [1100-1120] ORF | [964-984] ORF |
| 470 | CUGUGAAAGGAUCCUCCGGUU | AACCGGAGGAUCCUUUCACAG | | [1049-1069] ORF | [913-933] ORF |
| 471 | CUCGCAAGUGUGCAGAGUCUU | AAGACUCUGCACACUUGCGAG | | [925-945] ORF | [789-809] ORF |
| 472 | AGCACAGUUCUGUCCAGAGAA | UUCUCUGGACAGAACUGUGCU | | [7-27] 5′UTR | — |
| 473 | UGGUAUACUCACCACCUUUUU | AAAAAGGUGGUGAGUAUACCA | | [819-839] ORF | [683-703] ORF |
| 474 | CUGGUAUACUCACCACCUUUU | AAAAGGUGGUGAGUAUACCAG | | [818-838] ORF | [682-702] ORF |
| 475 | UGAGUUCAUCCGGAGGAGUUA | UAACUCCUCCGGAUGAACUCA | | [785-805] ORF | [649-669] ORF |
| 476 | AGUGAUCAUGACAAUAGCCUU | AAGGCUAUUGUCAUGAUCACU | | [740-760] ORF | [604-624] ORF |
| 477 | CUGGUCUCACUGGAGUGAUCA | UGAUCACUCCAGUGAGACCAG | | [727-747] ORF | [591-611] ORF |
| 478 | AUCUGCCUACAUACAGCUAUU | AAUAGCUGUAUGUAGGCAGAU | | [528-548] ORF | [392-412] ORF |
| 479 | GGCCUAUAUGAUCUGCCUACA | UGUAGGCAGAUCAUAUAGGCC | | [518-538] ORF | [382-402] ORF |
| 480 | AGCAAUUGGAUCACAACCUCA | UGAGGUUGUGAUCCAAUUGCU | | [481-501] ORF | [345-365] ORF |
| 481 | AAACUUAUUCAUUCCCAGGAA | UUCCUGGGAAUGAAUAAGUUU | | [40-60] 5′UTR | — |
| 482 | AGCGUCUGCUCUCUGCUUGAA | UUCAAGCAGAGAGCAGACGCU | | [368-388] ORF | [232-252] ORF |
| 483 | CACAAGAAAAAUCCUUGGGUC | GACCCAAGGAUUUUUCUUGUG | | [329-349] ORF | [193-213] ORF |
| 484 | GCCUUCCUGAAAUAUGAGAAG | CUUCUCAUAUUUCAGGAAGGC | | [291-311] ORF | [155-175] ORF |
| 485 | UUCAGUUUUGUUUCUGGUUGU | ACAACCAGAAACAAAACUGAA | | [236-256] ORF | [100-120] ORF |
| 488 | AACUAAUCCCAGGUGAUUUUA | UAAAAUCACCUGGGAUUAGUU | | [2273-2293] 3′UTR | — |
| 487 | GUAGCAAGGCUUGAUAGCAGA | UCUGCUAUCAAGCCUUGCUAC | | [2229-2249] 3′UTR | — |
| 488 | GCAACUUAAGAACUUCUGAAG | CUUCAGAAGUUCUUAAGUUGC | | [2127-2147] 3′UTR | — |
| 489 | UCGUUACUUGAGCUUCAGCAA | UUGCUGAAGCUCAAGUAACGA | | [2110-2130] 3′UTR | — |
| 490 | UCAAGCCUUGACUCCCUGGUA | UACCAGGGAGUCAAGGCUUGA | | [2065-2085] 3′UTR | [1725-1734] |
| | | | | | (10/10) 3′UTR |
| 491 | AGAAAGGUUCAAUUCUACUUC | GAAGUAGAAUUGAACCUUUCU | | [1863-1883] ORF | [1580-1600] ORF |
| 492 | CUGGAUCCUAGAAAGGUUCAA | UUGAACCUUUCUAGGAUCCAG | | [1854-1874] ORF | [1571-1591] ORF |
| 493 | CUGUCACCGAUAUUCCAGUCU | AGACUGGAAUAUCGGUGACAG | GP | [1835-1855] ORF | [1552-1572] ORF |
| 494 | UGGCAAAGAGCCUGCGCAAAU | AUUUGCGCAGGCUCUUUGCCA | | [1813-1833] ORF | [1530-1550] ORF |
| 495 | GCUCCAAACCACCUCUUGACA | UGUCAAGAGGUGGUUUGGAGC | | [186-206] 5′UTR | [50-70] 5′UTR |
| 496 | GGAGACCAAUGUGGGACAAUG | CAUUGUCCCACAUUGGUCUCC | Rat, Ms | [1723-1743] ORF | [1440-1460] ORF |
| 497 | CUGAAUCUUCCCUGUUGCCUA | UAGGCAACAGGGAAGAUUCAG | | [160-180] 5′UTR | [24-44] 5′UTR |
| 498 | CCGGAUUGCUGAAUCUUCCCU | AGGGAAGAUUCAGCAAUCCGG | | [152-172] 5′UTR | [16-36] 5′UTR |
| 499 | UCCUGGUUCAACAACCUGUUG | CAACAGGUUGUUGAACCAGGA | | [1542-1562] ORF | — |
| 500 | UUCCUGGUUCAACAACCUGUU | AACAGGUUGUUGAACCAGGAA | | [1541-1561] ORF | — |
| 501 | AUAUUCACCAAUUCCCAGGAU | AUCCUGGGAAUUGGUGAAUAU | | [1322-1342] ORF | [1186-1206] ORF |
| 502 | CUGGACAGAAAAUCUCAUAAG | CUUAUGAGAUUUUCUGUCCAG | Rat | [1286-1306] ORF | [1150-1170] ORF |
| 503 | GGGACUGGACAGAAAAUCUCA | UGAGAUUUUCUGUCCAGUCCC | Rat | [1282-1302] ORF | [1146-1166] ORF |
| 504 | CCAUUCAUAUCCGAGCAGCAG | CUGCUGCUCGGAUAUGAAUGG | | [1261-1281] ORF | [1125-1145] ORF |
| 505 | CAGAGGAAGAUUUCUUCUCCA | UGGAGAAGAAAUCUUCCUCUG | | [1243-1263] ORF | [1107-1127] ORF |
| 506 | CCUCUGCUCCAGAGGAAGAUU | AAUCUUCCUCUGGAGCAGAGG | | [1234-1254] ORF | [1098-1118] ORF |
| 507 | UGGAAUGGCAUCCUUUUACUU | AAGUAAAAGGAUGCCAUUCCA | | [1210-1230] ORF | [1074-1094] ORF |
| 508 | CUCCUGGAAUGGCAUCCUUUU | AAAAGGAUGCCAUUCCAGGAG | | [1206-1226] ORF | [1070-1090] ORF |
| 509 | UCUCCUGGAAUGGCAUCCUUU | AAAGGAUGCCAUUCCAGGAGA | | [1205-1225] ORF | [1069-1089] ORF |
| 510 | CCCUGAGUCUUGGAAGUGGAU | AUCCACUUCCAAGACUCAGGG | GP, Rat, | [1004-1024] ORF | [868-888] ORF |
| | | | Ms | | |
| 511 | GUAGGCGCCCUAAGUUUGAAG | CUUCAAACUUAGGGCGCCUAC | | [976-996] ORF | [840-860] ORF |
| 512 | GUCUCACUGGAGUGAUCAUGA | UCAUGAUCACUCCAGUGAGAC | | [730-750] ORF | [594-614] ORF |
| 513 | UGGUCUCACUGGAGUGAUCAU | AUGAUCACUCCAGUGAGACCA | | [728-748] ORF | [592-612] ORF |
| 514 | UUCCACAAGCUGGUGGCCUAU | AUAGGCCACCAGCUUGUGGAA | | [504-524] ORF | [368-388] ORF |
| 515 | GGAAUAAACUUAUUCAUUCCC | GGGAAUGAAUAAGUUUAUUCC | | [35-55] 5′UTR | — |
| 516 | GCGGAAUAAACUUAUUCAUUC | GAAUGAAUAAGUUUAUUCCGC | | [33-53] 5′UTR | — |
| 517 | CACUUUUGAUAUGGUCUUAUC | GAUAAGACCAUAUCAAAAGUG | | [2500-2520] 3′UTR | — |
| 518 | GGUUAACCACUGGUUUUCAGU | ACUGAAAACCAGUGGUUAACC | | [221-241] ORF | [85-105] ORF |
| 519 | UCCUGAGUUUUGGUUUGUAAU | AUUACAAACCAAAACUCAGGA | | [2314-2334] 3′UTR | — |
| 520 | AGAGUUAUACAACUAAUCCCA | UGGGAUUAGUUGUAUAACUCU | | [2263-2283] 3′UTR | — |
| 521 | GGAAACUGGGUGGUUAACCAC | GUGGUUAACCACCCAGUUUCC | | [210-230] ORF | [74-94] ORF |
| 522 | ACUGUAAAUCUUUCUGGACAG | CUGUCCAGAAAGAUUUACAGU | | [2200-2220] 3′UTR | — |
| 523 | CUAGAAAGGUUCAAUUCUACU | AGUAGAAUUGAACCUUUCUAG | | [1861-1881] ORF | [1578-1598] ORF |
| 524 | CCUAGAAAGGUUCAAUUCUAC | GUAGAAUUGAACCUUUCUAGG | | [1860-1880] ORF | [1577-1597] ORF |
| 525 | CCAAACCACCUCUUGACAAUG | CAUUGUCAAGAGGUGGUUUGG | | [189-209] | [53-73] |
| | | | | 5′UTR + ORF | 5′UTR + ORF |
| 526 | GGGAGUUUUCUUAUGUGGCCC | GGGCCACAUAAGAAAACUCCC | | [1784-1804] ORF | [1501-1521] ORF |
| 527 | GUUGCCUAGAAGGGCUCCAAA | UUUGGAGCCCUUCUAGGCAAC | | [173-193] 5′UTR | [37-57] 5′UTR |
| 528 | CUGAAACAGAAAACCUCCUUU | AAAGGAGGUUUUCUGUUUCAG | Rat | [1701-1721] ORF | [1418-1438] ORF |
| 529 | AGCAUUAAACUUUGACAAGGC | GCCUUGUCAAAGUUUAAUGCU | | [1661-1681] ORF | [1376-1396] ORF |
| 530 | GGUUUUCUAAACUACCGUCUC | GAGACGGUAGUUUAGAAAACC | | [1602-1622] ORF | — |
| 531 | CUAUUUCUACUGGAUCUGCAG | CUGCAGAUCCAGUAGAAAUAG | | [1505-1525] ORF | — |
| 532 | GCUUCUAUCUUGAAAUCCAUC | GAUGGAUUUCAAGAUAGAAGC | | [1437-1457] ORF | [1301-1321] ORF |
| 533 | CCAGUGAGGAUGUUUUCCAGU | ACUGGAAAACAUCCUCACUGG | | [1369-1389] ORF | [1233-1253] ORF |
| 534 | CCCAGGAUUGAAGUGGAUGGU | ACCAUCCACUUCAAUCCUGGG | | [1335-1355] ORF | [1199-1219] ORF |
| 535 | GCACCCAUCCAAAGUUUUGGA | UCCAAAACUUUGGAUGGGUGC | | [1112-1132] ORF | [976-996] ORF |
| 536 | UGUGAAAGGAUCCUCCGGUUU | AAACCGGAGGAUCCUUUCACA | | [1050-1070] ORF | [914-934] ORF |
| 537 | UCGCAAGUGUGCAGAGUCUUU | AAAGACUCUGCACACUUGCGA | | [926-946] ORF | [790-810] ORF |
| 538 | AGAGUCAUCCUCGCAAGUGUG | CACACUUGCGAGGAUGACUCU | | [916-936] ORF | [780-800] ORF |
| 539 | AGAGCAUGAAUGAGAGUCAUC | GAUGACUCUCAUUCAUGCUCU | | [904-924] ORF | [768-788] ORF |
| 540 | GAGAGCAUGAAUGAGAGUCAU | AUGACUCUCAUUCAUGCUCUC | | [903-923] ORF | [767-787] ORF |
| 541 | CACUGGAGUGAUCAUGACAAU | AUUGUCAUGAUCACUCCAGUG | | [734-754] ORF | [598-618] ORF |
| 542 | GGUUCUUGGCUAAAUCCCAUC | GAUGGGAUUUAGCCAAGAACC | | [660-680] ORF | [524-544] ORF |
| 543 | CAGCCUAUCUCAUGAUGAGAA | UUCUCAUCAUGAGAUAGGCUG | | [632-652] ORF | [496-516] ORF |
| 544 | CAAGAAAAAUCCUUGGGUCAA | UUGACCCAAGGAUUUUUCUUG | | [331-351] ORF | [195-215] ORF |
| 545 | ACAAGAAAAAUCCUUGGGUCA | UGACCCAAGGAUUUUUCUUGU | | [330-350] ORF | [194-214] ORF |
| 546 | AGUGUACAUGUUUACUGUCAC | GUGACAGUAAACAUGUACACU | | [2482-2502] 3′UTR | — |
| 547 | ACAGUGUACAUGUUUACUGUC | GACAGUAAACAUGUACACUGU | | [2480-2500] 3′UTR | — |
| 548 | ACACCAACAGUGUACAUGUUU | AAACAUGUACACUGUUGGUGU | | [2474-2494] 3′UTR | — |
| 549 | ACAACUAAUCCCAGGUGAUUU | AAAUCACCUGGGAUUAGUUGU | | [2271-2291] 3′UTR | — |
| 550 | UGGACAGCCAUGACUGUAGCA | UGCUACAGUCAUGGCUGUCCA | | [2214-2234] 3′UTR | — |
| 551 | CCAGUCUGGAUCCUAGAAAGG | CCUUUCUAGGAUCCAGACUGG | | [1849-1869] ORF | [1566-1586] ORF |
| 552 | CUCCAAACCACCUCUUGACAA | UUGUCAAGAGGUGGUUUGGAG | | [187-207] 5′UTR | [51-71] 5′UTR |
| 553 | GACAGGUCUGAAACAGAAAAC | GUUUUCUGUUUCAGACCUGUC | GP, Rat | [1694-1714] ORF | [1411-1431] ORF |
| 554 | UGACAGGUCUGAAACAGAAAA | UUUUCUGUUUCAGACCUGUCA | GP, Rat | [1693-1713] ORF | [1410-1430] ORF |
| 555 | UGACAUCGUGACAGGUCUGAA | UUCAGACCUGUCACGAUGUCA | | [1685-1705] ORF | [1402-1422] ORF |
| 556 | GAAUCUUCCCUGUUGCCUAGA | UCUAGGCAACAGGGAAGAUUC | | [162-182] 5′UTR | [26-46] 5′UTR |
| 557 | GGACAGCAAUAUUGUUGGUCA | UGACCAACAAUAUUGCUGUCC | | [1637-1657] ORF | — |
| 558 | ACCUGUUGACUUCCCUGGAAC | GUUCCAGGGAAGUCAACAGGU | | [1555-1575] ORF | — |
| 559 | CUGGUUCAACAACCUGUUGAC | GUCAACAGGUUGUUGAACCAG | | [1544-1564] ORF | — |
| 560 | UGUGCAGACCACAACCUCAAA | UUUGAGGUUGUGGUCUGCACA | | [1473-1493] ORF | [1337-1357] ORF |
| 561 | CAGAAUCCGGAUUGCUGAAUC | GAUUCAGCAAUCCGGAUUCUG | | [146-166] 5′UTR | [10-30] 5′UTR |
| 562 | GCCAGUGAGGAUGUUUUCCAG | CUGGAAAACAUCCUCACUGGC | | [1368-1388] ORF | [1232-1252] ORF |
| 563 | GGAUUGAAGUGGAUGGUCCCU | AGGGACCAUCCACUUCAAUCC | | [1339-1359] ORF | [1203-1223] ORF |
| 564 | CUCCAGAGGAAGAUUUCUUCU | AGAAGAAAUCUUCCUCUGGAG | | [1240-1260] ORF | [1104-1124] ORF |
| 565 | CCAAAGUUUUGGAAUUGCAGA | UCUGCAAUUCCAAAACUUUGG | | [1120-1140] ORF | [984-1004] ORF |
| 566 | UCCAAAGUUUUGGAAUUGCAG | CUGCAAUUCCAAAACUUUGGA | | [1119-1139] ORF | [983-1003] ORF |
| 567 | CACCCAUCCAAAGUUUUGGAA | UUCCAAAACUUUGGAUGGGUG | | [1113-1133] ORF | [977-997] ORF |
| 568 | AGCAGAAGGUUGUGAUUACCA | UGGUAAUCACAACCUUCUGCU | | [1081-1101] ORF | [945-965] ORF |
| 569 | GUGAAAGGAUCCUCCGGUUUU | AAAACCGGAGGAUCCUUUCAC | | [1051-1071] ORF | [915-935] ORF |
| 570 | GCACAGUUCUGUCCAGAGAAG | CUUCUCUGGACAGAACUGUGC | | [8-28] 5′UTR | — |
| 571 | CACUGUAGGCGCCCUAAGUUU | AAACUUAGGGCGCCUACAGUG | | [972-992] ORF | [836-856] ORF |
| 572 | UGAAUGAGAGUCAUCCUCGCA | UGCGAGGAUGACUCUCAUUCA | | [910-930] ORF | [774-794] ORF |
| 573 | CUGAGUUCAUCCGGAGGAGUU | AACUCCUCCGGAUGAACUCAG | | [784-804] ORF | [648-668] ORF |
| 574 | GAUCAUGACAAUAGCCUUGAU | AUCAAGGCUAUUGUCAUGAUC | | [743-763] ORF | [607-627] ORF |
| 575 | CUGCUAUAGCAGAAGCCGACA | UGUCGGCUUCUGCUAUAGCAG | | [578-598] ORF | [442-462] ORF |
| 576 | CUAUAUGAUCUGCCUACAUAC | GUAUGUAGGCAGAUCAUAUAG | | [521-541] ORF | [385-405] ORF |
| 577 | ACUUAUUCAUUCCCAGGAACU | AGUUCCUGGGAAUGAAUAAGU | | [42-62] 5′UTR | — |
| 578 | AGCCGCACACUGAGAAAGCAA | UUGCUUUCUCAGUGUGCGGCU | | [465-485] ORF | [329-349] ORF |
| 579 | CUGCUUCCUGUGUGUCGCAAU | AUUGCGACACACAGGAAGCAG | | [408-428] ORF | [272-292] ORF |
| 580 | ACCAACAGUGUACAUGUUUAC | GUAAACAUGUACACUGUUGGU | | [2476-2496] 3′UTR | — |
| 581 | GUGGUUAACCACUGGUUUUCA | UGAAAACCAGUGGUUAACCAC | | [219-239] ORF | [83-103] ORF |
| 582 | CCAGGUGAUUUUAUCAAUUCC | GGAAUUGAUAAAAUCACCUGG | | [2281-2301] 3′UTR | — |
| 583 | CCUUUCUCAGAAAAAUAACUG | CAGUUAUUUUUCUGAGAAAGG | | [2183-2203] 3′UTR | — |
| 584 | CCCAUGGAUCCUUUCUCAGAA | UUCUGAGAAAGGAUCCAUGGG | | [2174-2194] 3′UTR | — |
| 585 | GGGAAACUGGGUGGUUAACCA | UGGUUAACCACCCAGUUUCCC | | [209-229] ORF | [73-93] ORF |
| 586 | UUACUUGAGCUUCAGCAACUU | AAGUUGCUGAAGCUCAAGUAA | | [2113-2133] 3′UTR | — |
| 587 | UCCUAGAAAGGUUCAAUUCUA | UAGAAUUGAACCUUUCUAGGA | | [1859-1879] ORF | [1576-1596] ORF |
| 588 | UGGUCAUGCAGCAUUAAACUU | AAGUUUAAUGCUGCAUGACCA | GP, Rat, | [1652-1672] ORF | [1369-1389] ORF |
| | | | Ms | | |
| 589 | AGCAAUAUUGUUGGUCAUGCA | UGCAUGACCAACAAUAUUGCU | | [1641-1661] ORF | [1367-1378] |
| | | | | | (12/12) ORF |
| 590 | CGGAUUGCUGAAUCUUCCCUG | CAGGGAAGAUUCAGCAAUCCG | | [153-173] 5′UTR | [17-37] 5′UTR |
| 591 | CAACAACCUGUUGACUUCCCU | AGGGAAGUCAACAGGUUGUUG | | [1550-1570] ORF | — |
| 592 | GAUCUAUUUCUACUGGAUCUG | CAGAUCCAGUAGAAAUAGAUC | Ms | [1502-1522] ORF | — |
| 593 | CUGAUAGCACAGUUCUGUCCA | UGGACAGAACUGUGCUAUCAG | | [2-22] 5′UTR | — |
| 594 | GUGCAGACCACAACCUCAAAA | UUUUGAGGUUGUGGUCUGCAC | Gp | [1474-1494] ORF | [1338-1358] ORF |
| 595 | UCCAGAAUCCGGAUUGCUGAA | UUCAGCAAUCCGGAUUCUGGA | | [144-164] 5′UTR | [8-28] 5′UTR |
| 596 | CCAGUAUGAAGUGGCUGUGCU | AGCACAGCCACUUCAUACUGG | | [1385-1405] ORF | [1249-1269] ORF |
| 597 | UCACCAAUUCCCAGGAUUGAA | UUCAAUCCUGGGAAUUGGUGA | | [1326-1346] ORF | [1190-1210] ORF |
| 598 | AUCCAAAGUUUUGGAAUUGCA | UGCAAUUCCAAAACUUUGGAU | | [1118-1138] ORF | [982-1002] ORF |
| 599 | GGUUGUGAUUACCAAGGUUGU | ACAACCUUGGUAAUCACAACC | | [1088-1108] ORF | [952-972] ORF |
| 600 | CCUUGCACCGGUCAUUCUUUA | UAAAGAAUGACCGGUGCAAGG | | [1025-1045] ORF | [889-909] ORF |
| 601 | AGCAUGAAUGAGAGUCAUCCU | AGGAUGACUCUCAUUCAUGCU | | [906-926] ORF | [770-790] ORF |
| 602 | CUAUAUCCUUGGCUUAGGGAU | AUCCCUAAGCCAAGGAUAUAG | | [845-865] ORF | [709-729] ORF |
| 603 | GGUAUACUCACCACCUUUUUA | UAAAAAGGUGGUGAGUAUACC | | [820-840] ORF | [684-704] ORF |
| 604 | GGAGUGAUCAUGACAAUAGCC | GGCUAUUGUCAUGAUCACUCC | | [738-758] ORF | [602-622] ORF |
| 605 | CUCACUGGAGUGAUCAUGACA | UGUCAUGAUCACUCCAGUGAG | | [732-752] ORF | [596-616] ORF |
| 606 | GGGUUCUUGGCUAAAUCCCAU | AUGGGAUUUAGCCAAGAACCC | | [659-679] ORF | [523-543] ORF |
| 607 | GGUAGGUGUGUGUUUUUCACA | UGUGAAAAACACACACCUACC | | [68-88] 5′UTR | — |
| 608 | GCCGCACACUGAGAAAGCAAU | AUUGCUUUCUCAGUGUGCGGC | | [466-486] ORF | [330-350] ORF |
| 609 | CUGCUUGAAUUUUAACAGCAC | GUGCUGUUAAAAUUCAAGCAG | | [380-400] ORF | [244-264] ORF |
| 610 | ACACAAGAAAAAUCCUUGGGU | ACCCAAGGAUUUUUCUUGUGU | | [328-348] ORF | [192-212] ORF |
| 611 | ACAGAAGAUUUCCUAAGUAGG | CCUACUUAGGAAAUCUUCUGU | | [2355-2375] 3′UTR | — |
| 612 | GAACUUCUGAAGUUCUUAAAG | CUUUAAGAACUUCAGAAGUUC | | [2136-2156] 3′UTR | — |
| 613 | CAACUUAAGAACUUCUGAAGU | ACUUCAGAAGUUCUUAAGUUG | | [2128-2148] 3′UTR | — |
| 614 | UGAGCUUCAGCAACUUAAGAA | UUCUUAAGUUGCUGAAGCUCA | | [2118-2138] 3′UTR | — |
| 615 | CACCGAUAUUCCAGUCUGGAU | AUCCAGACUGGAAUAUCGGUG | | [1839-1859] ORF | [1556-1576] ORF |
| 616 | UCACCGAUAUUCCAGUCUGGA | UCCAGACUGGAAUAUCGGUGA | | [1838-1858] ORF | [1555-1575] ORF |
| 617 | UCCAAACCACCUCUUGACAAU | AUUGUCAAGAGGUGGUUUGGA | | [188-208] | [52-72] |
| | | | | 5′UTR + ORF | 5′UTR + ORF |
| 618 | ACCAAUGUGGGACAAUGAGUU | AACUCAUUGUCCCACAUUGGU | Rat, Ms | [1727-1747] ORF | [1444-1464] ORF |
| 619 | AGGUGCCUUUUCCUGGUUCAA | UUGAACCAGGAAAAGGCACCU | GP | [1532-1552] ORF | — |
| 620 | GUGAGGAUGUUUUCCAGUAUG | CAUACUGGAAAACAUCCUCAC | Ms | [1372-1392] ORF | [1236-1256] ORF |
| 621 | CCAAUUCCCAGGAUUGAAGUG | CACUUCAAUCCUGGGAAUUGG | | [1329-1349] ORF | [1193-1213] ORF |
| 622 | ACCAAUUCCCAGGAUUGAAGU | ACUUCAAUCCUGGGAAUUGGU | | [1328-1348] ORF | [1192-1212] ORF |
| 623 | CAGAUGAACAAGCGUGGCUUC | GAAGCCACGCUUGUUCAUCUG | | [1137-1157] ORF | [1001-1021] ORF |
| 624 | ACCAAGGUUGUUAUGCACCCA | UGGGUGCAUAACAACCUUGGU | | [1096-1118] ORF | [962-982] ORF |
| 625 | AGGUUGUGAUUACCAAGGUUG | CAACCUUGGUAAUCACAACCU | | [1087-1107] ORF | [951-971] ORF |
| 626 | CUCCCAGCAGAAGGUUGUGAU | AUCACAACCUUCUGCUGGGAG | | [1076-1096] ORF | [940-960] ORF |
| 627 | UCCUUGCACCGGUCAUUCUUU | AAAGAAUGACCGGUGCAAGGA | | [1024-1044] ORF | [888-908] ORF |
| 628 | CCUUGAUUCUCAUGGUAACUU | AAGUUACCAUGAGAAUCAAGG | GP, | [757-777] ORF | [621-641] ORF |
| | | | Rabbit | | |
| 629 | UCAUGACAAUAGCCUUGAUUC | GAAUCAAGGCUAUUGUCAUGA | | [745-765] ORF | [609-629] ORF |
| 630 | AUCAUGACAAUAGCCUUGAUU | AAUCAAGGCUAUUGUCAUGAU | | [744-764] ORF | [608-628] ORF |
| 631 | UGAUCAUGACAAUAGCCUUGA | UCAAGGCUAUUGUCAUGAUCA | | [742-762] ORF | [606-626] ORF |
| 632 | GUAGGUGUGUGUUUUUCACAU | AUGUGAAAAACACACACCUAC | | [69-89] 5′UTR | — |
| 633 | GGGUAGGUGUGUGUUUUUCAC | GUGAAAAACACACACCUACCC | | [67-87] 5′UTR | — |
| 634 | GGUGGCCUAUAUGAUCUGCCU | AGGCAGAUCAUAUAGGCCACC | | [515-535] ORF | [379-399] ORF |
| 635 | CAACCUCACCUUCCACAAGCU | AGCUUGUGGAAGGUGAGGUUG | | [494-514] ORF | [358-378] ORF |
| 636 | GAAGGCGGAAUAAACUUAUUC | GAAUAAGUUUAUUCCGCCUUC | | [29-49] 5′UTR | — |
| 637 | GGUCUUAUCCAGUGUGAACAG | CUGUUCACACUGGAUAAGACC | | [2512-2532] 3′UTR | — |
| 638 | UGAUAUGGUCUUAUCCAGUGU | ACACUGGAUAAGACCAUAUCA | | [2506-2526] 3′UTR | — |
| 639 | GUCUGGAUCCUAGAAAGGUUC | GAACCUUUCUAGGAUCCAGAC | | [1852-1872] ORF | [1569-1589] ORF |
| 640 | UCCAGUCUGGAUCCUAGAAAG | CUUUCUAGGAUCCAGACUGGA | | [1848-1888] ORF | [1565-1585] ORF |
| 641 | GCUGUCACCGAUAUUCCAGUC | GACUGGAAUAUCGGUGACAGC | GP | [1834-1854] ORF | [1551-1571] ORF |
| 642 | UGGGAGACCAAUGUGGGACAA | UUGUCCCACAUUGGUCUCCCA | Rat, Ms | [1721-1741] ORF | [1438-1458] ORF |
| 643 | UCGUGACAGGUCUGAAACAGA | UCUGUUUCAGACCUGUCACGA | | [1690-1710] ORF | [1407-1427] ORF |
| 644 | ACAUCGUGACAGGUCUGAAAC | GUUUCAGACCUGUCACGAUGU | | [1687-1707] ORF | [1404-1424] ORF |
| 645 | ACAAGGCCACUGACAUCGUGA | UCACGAUGUCAGUGGCCUUGU | | [1675-1695] ORF | [1392-1412] ORF |
| 646 | UGACAAGGCCACUGACAUCGU | ACGAUGUCAGUGGCCUUGUCA | | [1673-1693] ORF | [1390-1410] ORF |
| 647 | UGAAAGGAUCCUCCGGUUUUA | UAAAACCGGAGGAUCCUUUCA | | [1052-1072] ORF | [916-936] ORF |
| 648 | GUCUUCUGGUAUACUCACCAC | GUGGUGAGUAUACCAGAAGAC | Rabbit | [813-833] ORF | [677-697] ORF |
| 649 | UGACAAUAGCCUUGAUUCUCA | UGAGAAUCAAGGCUAUUGUCA | | [748-768] ORF | [612-632] ORF |
| 650 | UCACUGGAGUGAUCAUGACAA | UUGUCAUGAUCACUCCAGUGA | | [733-753] ORF | [597-617] ORF |
| 651 | CACAAGCUGGUGGCCUAUAUG | CAUAUAGGCCACCAGCUUGUG | | [507-527] ORF | [371-391] ORF |
| 652 | CGCACACUGAGAAAGCAAUUG | CAAUUGCUUUCUCAGUGUGCG | | [468-488] ORF | [332-352] ORF |
| 653 | CAGGUGAUUUUAUCAAUUCCA | UGGAAUUGAUAAAAUCACCUG | | [2282-2302] 3′UTR | — |
| 654 | GCUUGAUAGCAGAGGUUUGGU | ACCAAACCUCUGCUAUCAAGC | | [2237-2257] 3′UTR | — |
| 655 | UACUUGAGCUUCAGCAACUUA | UAAGUUGCUGAAGCUCAAGUA | | [2114-2134] 3′UTR | — |
| 656 | AAGCCUUGACUCCCUGGUAUU | AAUACCAGGGAGUCAAGGCUU | | [2067-2087] 3′UTR | — |
| 657 | ACAGAAAACCUCCUUUGGGAG | CUCCCAAAGGAGGUUUUCUGU | Rat | [1706-1726] ORF | [1423-1443] ORF |
| 658 | CGUGACAGGUCUGAAACAGAA | UUCUGUUUCAGACCUGUCACG | | [1691-1711] ORF | [1408-1428] ORF |
| 659 | GGGACAGCAAUAUUGUUGGUC | GACCAACAAUAUUGCUGUCCC | | [1636-1656] ORF | — |
| 660 | UCACCGGAUGGGACAGCAAUA | UAUUGCUGUCCCAUCCGGUGA | | [1627-1647] ORF | — |
| 661 | CAGGUGCCUUUUCCUGGUUCA | UGAACCAGGAAAAGGCACCUG | GP | [1531-1551] ORF | — |
| 662 | CAGUGAGGAUGUUUUCCAGUA | UACUGGAAAACAUCCUCACUG | Ms | [1370-1390] ORF | [1234-1254] ORF |
| 663 | ACAGCCAGUGAGGAUGUUUUC | GAAAACAUCCUCACUGGCUGU | | [1365-1385] ORF | [1229-1249] ORF |
| 664 | GGUUGUUAUGCACCCAUCCAA | UUGGAUGGGUGCAUAACAACC | | [1103-1123] ORF | [967-987] ORF |
| 665 | UCAAACAGAGGAGAGCAUGAA | UUCAUGCUCUCCUCUGUUUGA | | [893-913] ORF | [757-777] ORF |
| 666 | CAAGCUGGUGGCCUAUAUGAU | AUCAUAUAGGCCACCAGCUUG | | [509-529] ORF | [373-393] ORF |
| 667 | CCAACAGUGUACAUGUUUACU | AGUAAACAUGUACACUGUUGG | | [2477-2497] 3′UTR | — |
| 668 | CCAGUGUUACCAUCUCCUGAG | CUCAGGAGAUGGUAACACUGG | | [2300-2320] 3′UTR | — |
| 669 | CAAGCCUUGACUCCCUGGUAU | AUACCAGGGAGUCAAGGCUUG | | [2066-2086] 3′UTR | — |
| 670 | CACCGGAUGGGACAGCAAUAU | AUAUUGCUGUCCCAUCCGGUG | | [1628-1648] ORF | — |
| 671 | ACAGGUGCCUUUUCCUGGUUC | GAACCAGGAAAAGGCACCUGU | GP | [1530-1550] ORF | — |
| 672 | GGUCAAACAGAGGAGAGCAUG | CAUGCUCUCCUCUGUUUGACC | | [891-911] ORF | [755-775] ORF |
| 673 | CACCAACAGUGUACAUGUUUA | UAAACAUGUACACUGUUGGUG | | [2475-2495] 3′UTR | — |
| 674 | AGGUGAUUUUAUCAAUUCCAG | CUGGAAUUGAUAAAAUCACCU | | [2283-2303] 3′UTR | — |
| 675 | CUUAAAGCCCAUGGAUCCUUU | AAAGGAUCCAUGGGCUUUAAG | | [2167-2187] 3′UTR | — |
| 676 | ACUUAAGAACUUCUGAAGUUC | GAACUUCAGAAGUUCUUAAGU | | [2130-2150] 3′UTR | — |
| 677 | AAACCUCCUUUGGGAGACCAA | UUGGUCUCCCAAAGGAGGUUU | Rat | [1711-1731] ORF | [1428-1448] ORF |
| 678 | UGAAACAGAAAACCUCCUUUG | CAAAGGAGGUUUUCUGUUUCA | Rat | [1702-1722] ORF | [1419-1439] ORF |
| 679 | UCUGAAACAGAAAACCUCCUU | AAGGAGGUUUUCUGUUUCAGA | Rat | [1700-1720] ORF | [1417-1437] ORF |
| 680 | CAGGUCUGAAACAGAAAACCU | AGGUUUUCUGUUUCAGACCUG | GP, Rat | [1696-1716] ORF | [1413-1433] ORF |
| 681 | UGCUCCAGAGGAAGAUUUCUU | AAGAAAUCUUCCUCUGGAGCA | | [1238-1258] ORF | [1102-1122] ORF |
| 682 | UCCCAGCAGAAGGUUGUGAUU | AAUCACAACCUUCUGCUGGGA | | [1077-1097] ORF | [941-961] ORF |
| 683 | GGACAGCCAUGACUGUAGCAA | UUGCUACAGUCAUGGCUGUCC | | [2215-2235] 3′UTR | — |
| 684 | CUAUCUUGAAAUCCAUCUGGU | ACCAGAUGGAUUUCAAGAUAG | | [1441-1461] ORF | [1305-1325] ORF |
| 685 | GAGGAUGUUUUCCAGUAUGAA | UUCAUACUGGAAAACAUCCUC | Ms | [1374-1394] ORF | [1238-1258] ORF |
| 686 | CUACUACACAAGAAAAAUCCU | AGGAUUUUUCUUGUGUAGUAG | | [323-343] ORF | [187-207] ORF |
| 687 | UGAGGAUGUUUUCCAGUAUGA | UCAUACUGGAAAACAUCCUCA | Ms | [1373-1393] ORF | [1237-1257] ORF |
| 688 | CCCAGCAGAAGGUUGUGAUUA | UAAUCACAACCUUCUGCUGGG | | [1078-1096] ORF | [942-962] ORF |
| 689 | CUGUUUGUGGAUGCCUUCCUG | CAGGAAGGCAUCCACAAACAG | | [279-299] ORF | [143-163] ORF |
| 690 | UGGUUUUCAGUUUUGUUUCUG | CAGAAACAAAACUGAAAACCA | | [231-251] ORF | [95-115] ORF |
| 691 | GAGCUUCAGCAACUUAAGAAC | GUUCUUAAGUUGCUGAAGCUC | | [2119-2139] 3′UTR | — |
| 692 | GAAAGGAUCCUCCGGUUUUAC | GUAAAACCGGAGGAUCCUUUC | | [1053-1073] ORF | [917-937] ORF |
| 693 | GAGUCUUGGAAGUGGAUCCUU | AAGGAUCCACUUCCAAGACUC | Ms | [1008-1028] ORF | [872-892] ORF |
| 694 | AUCCAGUGUGAACAGCAAUUU | AAAUUGCUGUUCACACUGGAU | | [2518-2538] 3′UTR | — |
| 695 | AGCUUCAGCAACUUAAGAACU | AGUUCUUAAGUUGCUGAAGCU | | [2120-2140] 3′UTR | — |
| 696 | GCUCCAGAGGAAGAUUUCUUC | GAAGAAAUCUUCCUCUGGAGC | | [1239-1259] ORF | [1103-1123] ORF |
| 697 | UGGUAACUUCAGCUACUGAGU | ACUCAGUAGCUGAAGUUACCA | | [769-789] ORF | [633-653] ORF |
| 698 | ACCGGAUGGGACAGCAAUAUU | AAUAUUGCUGUCCCAUCCGGU | | [1629-1649] ORF | — |
| 699 | UGCUGGUCUCACUGGAGUGAU | AUCACUCCAGUGAGACCAGCA | | [725-745] ORF | [589-609] ORF |
| 700 | CUGGUUUUCAGUUUUGUUUCU | AGAAACAAAACUGAAAACCAG | | [230-250] ORF | [94-114] ORF |
| 701 | UUAUCCAGUGUGAACAGCAAU | AUUGCUGUUCACACUGGAUAA | | [2516-2536] 3′UTR | — |
| 702 | GGUUUUCAGUUUUGUUUCUGG | CCAGAAACAAAACUGAAAACC | | [232-252] ORF | [96-116] ORF |
| 703 | CACUGGUUUUCAGUUUUGUUU | AAACAAAACUGAAAACCAGUG | | [228-248] ORF | [92-112] ORF |
| 704 | CUCCUUUGGGAGACCAAUGUG | CACAUUGGUCUCCCAAAGGAG | Rat | [1715-1735] ORF | [1432-1452] ORF |
| 705 | GUGCUGGUUGGAGCAGGAAUU | AAUUCCUGCUCCAACCAGCAC | | [1401-1421] ORF | [1265-1285] ORF |
| 706 | GGAUGUUUUCCAGUAUGAAGU | ACUUCAUACUGGAAAACAUCC | Ms | [1376-1396] ORF | [1240-1260] ORF |
| 707 | UGCUGGUUGGAGCAGGAAUUG | CAAUUCCUGCUCCAACCAGCA | | [1402-1422] ORF | [1268-1286] ORF |
| 708 | UGAGUCUUGGAAGUGGAUCCU | AGGAUCCACUUCCAAGACUCA | Rat, Ms | [1007-1027] ORF | [871-891] ORF |
| 709 | AACUUCAGCUACUGAGUUCAU | AUGAACUCAGUAGCUGAAGUU | | [773-793] ORF | [637-657] ORF |
| 710 | GGUAACUUCAGCUACUGAGUU | AACUCAGUAGCUGAAGUUACC | | [770-790] ORF | [634-654] ORF |

TABLE E
19-mer oligomers NOX2 (NADPH oxidase 2; CYBB)

| | | | | |
|---|---|---|---|---|
| | | | | Human-6996020 |
| Number | Sense siRNA | AntiSense siRNA | Other Sp | ORF: 15-1727 |

| | | | | |
|---|---|---|---|---|
| 1 | GGGUUUUCUUUUUGUUCUU | AAGAACAAAAAGAAAACCC | | [3055-3073] 3′UTR |
| 2 | CCAGAUCAUUAGGACAUAA | UUAUGUCCUAAUGAGCUGG | | [2240-2258] 3′UTR |
| 3 | GCCUCAUAUUAAUUAUCAC | GUGAUAAUUAAUAUGAGGC | | [568-586] ORF |
| 4 | GCAUAAUUAAAACCAGCUU | AAGCUGGUUUUAAUUAUGC | | [3800-3818] 3′UTR |
| 5 | GCAGGGUUUUCUUUUUGUU | AACAAAAAGAAAACCCUGC | | [3052-3070] 3′UTR |
| 6 | CCAACUGGGAUAAUGAAUU | AAUUCAUUAUCCCAGUUGG | | [1555-1573] ORF |
| 7 | GGGUCUCAUAUUGGUCAUU | AAUGACCAAUAUGAGACCC | | [3291-3309] 3′UTR |
| 8 | GGAUAAUGAAUUCAAGACA | UGUCUUGAAUUCAUUAUCC | | [1562-1580] ORF |
| 9 | GCCUGAAUUUCAACUGCAU | AUGCAGUUGAAAUUCAGGC | Rat | [190-206] ORF |
| 10 | CGCUGUGCCUCAUAUUAAU | AUUAAUAUGAGGCACAGCG | | [562-580] ORF |
| 11 | CUAACCUAUGUGCAUAAUU | AAUUAUGCACAUAGGUUAG | | [3769-3807] 3′UTR |
| 12 | CAAUUCAUCUAUUCAACAG | CUGUUGAAUAGAUGAAUUG | | [3853-3871] 3′UTR |
| 13 | GUGUGACUAAUGGUUUAUU | AAUAAACCAUUAGUCACAC | | [2892-2910] 3′UTR |
| 14 | ACCACACAAUGCCAAGAUA | UAUCUUGGCAUUGUGUGGU | | [2473-2491] 3′UTR |
| 15 | GCCUCAGAAUCCUCAAAAU | AUUUUGAGGAUUCUGAGGC | | [2118-2136] 3′UTR |
| 16 | GAAACCCUGAGUAAACAAA | UUUGUUUACUCAGGGUUUC | | [1644-1662] ORF |
| 17 | CCAACUAAGUUAUGAAAGU | ACUUUCAUAACUUAGUUGG | | [3866-3884] 3′UTR |
| 18 | CCAUUGCACAUCUAUUUAA | UUAAAUAGAUGUGCAAUGG | | [381-379] ORF |
| 19 | CAGGUAUCAAUUCAUCUAU | AUAGAUGAAUUGAUACCUG | | [3646-3664] 3′UTR |
| 20 | CGAAUAUAGCCUUUCUGAA | UUCAGAAAGGCUAUAUUCG | | [3568-3586] 3′UTR |
| 21 | GGCCUGCUGUGCGAAUAUA | UAUAUUCGCACAGCAGGCC | | [3557-3575] 3′UTR |
| 22 | AGGGUUUUCUUUUUGUUCU | AGAACAAAAAGAAAACCCU | | [3054-3072] 3′UTR |
| 23 | CCCUUAUGAAUAUUGAACU | AGUUCAAUAUUCAUAAGGG | | [2775-2793] 3′UTR |
| 24 | GUAAUUUCCUGCCCAAAGU | ACUUUGGGCAGGAAAUUAG | | [2740-2758] 3′UTR |
| 25 | CAACACCCUAAUACCAGAA | UUCUGGUAUUAGGGUGUGG | Rabbit | [1590-1608] ORF |
| 26 | GGGAUAAUGAAUUCAAGAC | GUCUUGAAUUCAUUAUCCC | | [1561-1579] ORF |
| 27 | ACUGGGAUAAUGAAUUCAA | UUGAAUUCAUUAUCCCAGU | | [1558-1576] ORF |
| 28 | CCAUCCGGAGGUCUUACUU | AAGUAAGACCUCCGGAUGG | | [601-619] ORF |
| 29 | GUGCCUCAUAUUAAUUAUC | GAUAAUUAAUAUGAGGCAC | | [566-584] ORF |
| 30 | CUGUGCCUCAUAUUAAUUA | UAAUUAAUAUGAGGCACAG | | [564-582] ORF |
| 31 | CCGAGUCAAUAAUUCUGAU | AUCAGAAUUAUUGACUCGG | | [401-419] ORF |
| 32 | GGCUUUGUAUGUGAAUAAU | AUUAUUCACAUACAAAGCC | | [4171-4189] 3′UTR |
| 33 | UCUAACCUAUGUGCAUAAU | AUUAUGCACAUAGGUUAGA | | [3768-3806] 3′UTR |
| 34 | UCAGGUAUCAAUUCAUCUA | UAGAUGAAUUGAUACCUGA | | [3645-3683] 3′UTR |
| 35 | CCCAGAUUUCUUAAAGUUU | AAACUUUAAGAAAUCUGGG | | [3316-3334] 3′UTR |
| 36 | GAGCCACAAAUGUUUAGAA | UUCUAAACAUUUGUGGCUC | | [3128-3146] 3′UTR |
| 37 | AGAGCAGGGUUUUCUUUUU | AAAAAGAAAACCCUGCUCU | | [3049-3067] 3′UTR |
| 38 | UCAUCAUCAUCAUCCUAAA | UUUAGGAUGAUGAUGAUGA | | [2920-2838] 3′UTR |
| 39 | CUGUGUGACUAAUGGUUUA | UAAACCAUUAGUCACACAG | | [2890-2908] 3′UTR |
| 40 | CUGGGAUAAUGAAUUCAAG | CUUGAAUUCAUUAUCCCAG | | [1559-1577] ORF |
| 41 | UCGAGCUACAGAUGAAGAA | UUCUUCAUCUGUAGCUCGA | | [937-955] ORF |
| 42 | GGUCUUACUUUGAAGUCUU | AAGACUUCAAAGUAAGACC | | [610-628] ORF |
| 43 | GGGAAUUCUUAAAACUGUG | CACAGUUUUAAGAAUUCCC | | [4221-4239] 3′UTR |
| 44 | GAGUGCUUGUCAUUAUAAA | UUUAUAAUGACAAGCACUC | | [3935-3953] 3′UTR |
| 45 | GGGACUAGGAUGAACUAAA | UUUAGUUCAUCCUAGUCCC | | [3453-3471] 3′UTR |
| 46 | GGAGAAAAGAGAAUGGGAG | CUCCCAUUCUCUUUUCUCC | | [3228-3246] 3′UTR |
| 47 | GGAGCCACAAAUGUUUAGA | UCUAAACAUUUGUGGCUCC | | [3127-3145] 3′UTR |
| 48 | CCCUUAUUUUCCGUAAAUA | UAUUUACGGAAAAUAAGGG | | [2850-2868] 3′UTR |
| 49 | GCCCUUAUGAAUAUUGAAC | GUUCAAUAUUCAUAAGGGC | | [2774-2792] 3′UTR |
| 50 | AACCACACAAUGCCAAGAU | AUCUUGGCAUUGUGUGGUU | | [2472-2490] 3′UTR |
| 51 | UGAAUUUAGAACCACACAA | UUGUGUGGUUCUAAAUUCA | | [2463-2481] 3′UTR |
| 52 | ACAUUUAUGUGGAGCUCUA | UAGAGCUCCACAUAAAUGU | | [1892-1910] 3′UTR |
| 53 | UGAAACCCUGAGUAAACAA | UUGUUUACUCAGGGUUUCA | | [1643-1661] ORF |
| 54 | CUGAAACCCUGAGUAAACA | UGUUUACUCAGGGUUUCAG | | [1642-1660] ORF |
| 55 | GGCUGUGCAUAAUAUAACA | UGUUAUAUUAUGCACAGCC | | [722-740] ORF |
| 56 | GUCUUACUUUGAAGUCUUU | AAAGACUUCAAAGUAAGAC | | [611-629] ORF |
| 57 | UUAAAACUGUGCUCAACUA | UAGUUGAGCACAGUUUUAA | | [4229-4247] 3′UTR |
| 58 | AGGCUUUGUAUGUGAAUAA | UUAUUCACAUACAAAGCCU | | [4170-4188] 3′UTR |
| 59 | GCAUUUAUUAACAGGUACU | AGUACCUGUUAAUAAAUGC | | [4053-4071] 3′UTR |
| 60 | UGUGCAUAAUUAAAACCAG | CUGGUUUUAAUUAUGCACA | | [3797-3815] 3′UTR |
| 61 | UUCUAACCUAUGUGCAUAA | UUAUGCACAUAGGUUAGAA | | [3787-3805] 3′UTR |
| 62 | UCAAUUCUUUACACCAAAG | CUUUGGUGUAAAGAAUUGA | | [3716-3734] 3′UTR |
| 63 | GAAUAUAGCCUUUCUGAAA | UUUCAGAAAGGCUAUAUUC | | [3569-3587] 3′UTR |
| 64 | GGUCUCAUAUUGGUCAUUC | GAAUGACCAAUAUGAGACC | | [3292-3310] 3′UTR |
| 65 | CCAAGAGCAGGGUUUUCUU | AAGAAAACCCUGCUCUUGG | | [3046-3064] 3′UTR |
| 66 | CCACACAAUGCCAAGAUAG | CUAUCUUGGCAUUGUGUGG | | [2474-2492] 3′UTR |
| 67 | UGGAUUGAUUGGAGUUAAU | AUUAACUCCAAUCAAUCCA | | [2373-2391] 3′UTR |
| 68 | CCAGGUUUGAUACUCUUUC | GAAAGAGUAUCAAACCUGG | | [2085-2103] 3′UTR |
| 69 | CUCCAGGUUUGAUACUCUU | AAGAGUAUCAAACCUGGAG | | [2083-2101] 3′UTR |
| 70 | UCAAAAGGUUCAGUAACUU | AAGUUACUGAACCUUUUGA | | [2023-2041] 3′UTR |
| 71 | GGAUCAAAAGGUUCAGUAA | UUACUGAACCUUUUGAUCC | | [2020-2038] 3′UTR |
| 72 | GCAUUUCAUUUUCAACAAG | CUUGUUGAAAAUGAAAUGC | | [1697-1715] ORF |
| 73 | GAAUUCAAGACAAUUGCAA | UUGCAAUUGUCUUGAAUUC | | [1569-1587] ORF |
| 74 | GGAUGAGUCUCAGGCCAAU | AUUGGCCUGAGACUCAUCC | | [1463-1481] ORF |
| 75 | GCCAGAUGCAGGAAAGGAA | UUCCUUUCCUGCAUCUGGC | | [1402-1420] ORF |
| 76 | GAGCUACAGAUGAAGAAGA | UCUUCUUCAUCUGUAGCUC | | [939-957] ORF |
| 77 | GCGGUUUUGGCGAUCUCAA | UUGAGAUCGCCAAAACCGC | | [872-890] ORF |
| 78 | GGAGGUCUUACUUUGAAGU | ACUUCAAAGUAAGACCUCC | Rat, Rabbit | [607-625] ORF |
| 79 | CGGAGGUCUUACUUUGAAG | CUUCAAAGUAAGACCUCCG | Rat, Rabbit | [606-624] ORF |
| 80 | AUCACUUCCUCCACCAAAA | UUUUGGUGGAGGAAGUGAU | | [582-600] ORF |
| 81 | GCAGGCUUUGUAUGUGAAU | AUUCACAUACAAAGCCUGC | | [4168-4186] 3′UTR |
| 82 | AGAGUGCUUGUCAUUAUAA | UUAUAAUGACAAGCACUCU | | [3934-3962] 3′UTR |
| 83 | CUAUCUUCAAAAGCAACUA | UAGUUGGUUUUGAAGAUAG | | [3854-3872] 3′UTR |
| 84 | GCCCUUUCUAACCUAUGUG | CACAUAGGUUAGAAAGGGC | | [3782-3800] 3′UTR |
| 85 | ACCUCAGGUAUCAAUUCAU | AUGAAUUGAUACCUGAGGU | | [3642-3860] 3′UTR |
| 86 | AGCAGGGUUUUCUUUUUGU | ACAAAAAGAAAACCCUGCU | | [3051-3069] 3′UTR |
| 87 | AUAAAAUGGUGGCAUGGAU | AUCCAUGCCACCAUUUUAU | | [316-334] ORF |
| 88 | UGUGUGACUAAUGGUUUAU | AUAAACCAUUAGUCACACA | | [2891-2909] 3′UTR |
| 89 | AGCUAGCCCUUAUGAAUAU | AUAUUCAUAAGGGCUAGCU | | [2769-2787] 3′UTR |
| 90 | GACUAAUUUCCUGGCCAAA | UUUGGGCAGGAAAUUAGUC | | [2738-2756] 3′UTR |
| 91 | GCAUGAAAAAAGAGGGUUG | CAACCCUCUUUUUUCAUGC | | [2646-2664] 3′UTR |
| 92 | ACAAUGCCAAGAUAGAAUU | AAUUCUAUCUUGGCAUUGU | | [2478-2496] 3′UTR |
| 93 | CACAAUGCCAAGAUAGAAU | AUUCUAUCUUGGCAUUGUG | | [2477-2495] 3′UTR |
| 94 | GAACCACACAAUGCCAAGA | UCUUGGCAUUGUGUGGUUC | | [2471-2489] 3′UTR |
| 95 | ACAUAAUACAUGUUGAGAG | CUCUCAACAUGUAUUAUGU | | [2253-2271] 3′UTR |
| 96 | GGAGUAGCUAUAUAUUUCU | AGAAAUAUAUAGCUACUCC | | [2173-2191] 3′UTR |
| 97 | UGCCUCAGAAUCCUCAAAA | UUUUGAGGAUUCUGAGGCA | | [2117-2135] 3′UTR |
| 98 | GGAGCUCUAUGGUUUUGAG | CUCAAAACCAUAGAGCUCC | | [1902-1920] 3′UTR |
| 99 | GCUGAAACCCUGAGUAAAC | GUUUACUCAGGGUUUCAGC | | [1641-1659] ORF |
| 100 | UCAACACCCUAAUACCAGA | UCUGGUAUUAGGGUGUUGA | Rabbit | [1589-1607] ORF |
| 101 | CCACCUAAGUUCUUUUACA | UGUAAAAGAACUUAGGUGG | | [120-138] ORF |
| 102 | CGAGCUACAGAUGAAGAAG | CUUCUUCAUCUGUAGCUCG | | [938-958] ORF |
| 103 | GCUGUGCCUCAUAUUAAUU | AAUUAAUAUGAGGCACAGC | | [563-581] ORF |
| 104 | GGCAAAAUGAAAGUUAUCU | AGAUAACUUUCAUUUUGCC | | [454-472] ORF |
| 105 | CAGGCAAAAUGAAAGUUAU | AUAACUUUCAUUUUGCCUG | | [452-470] ORF |
| 106 | ACAGGCAAAAUGAAAGUUA | UAACUUUCAUUUUGCCUGU | | [451-469] ORF |
| 107 | GUCAAUAAUUCUGAUCCUU | AAGGAUCAGAAUUAUUGAC | | [405-423] ORF |
| 108 | CCCGAGUCAAUAAUUCUGA | UCAGAAUUAUUGACUCGGG | | [400-418] ORF |
| 109 | CUGUGCUCAACUAUUAAAA | UUUUAAUAGUUGAGCACAG | | [4235-4253] 3′UTR |
| 110 | GAAUUCUUAAAACUGUGCU | AGCACAGUUUUAAGAAUUC | | [4223-4241] 3′UTR |
| 111 | GGAAUUCUUAAAACUGUGC | GCACAGUUUUAAGAAUUCC | | [4222-4240] 3′UTR |
| 112 | UCUUAGUUCUUCUGCUUUU | AAAAGCAGAAGAACUAAGA | | [4120-4138] 3′UTR |
| 113 | GUGCUUGUCAUUAUAAAAG | CUUUUAUAAUGACAAGCAC | | [3937-3955] 3′UTR |
| 114 | GUUAUAAGGAGUCUGAGAA | UUCUGAGACUCCUUAUAAC | | [3758-3776] 3′UTR |
| 115 | GCUCUGUUUAUUGUUUCAA | UUGAAACAAUAAACAGAGC | | [3701-3719] 3′UTR |
| 116 | CCUCAGGUAUCAAUUCAUC | GAUGAAUUGAUACCUGAGG | | [3643-3661] 3′UTR |
| 117 | GCCUUUCUGAAAUGUACCA | UGGUACAUUUCAGAAAGGC | | [3576-3594] 3′UTR |
| 118 | GGAUGAACUAAAUAAGAAC | GUUCUUAUUUAGUUCAUCC | | [3460-3478] 3′UTR |
| 119 | CACAAAUGUUUAGAACUCU | AGAGUUCUAAACAUUUGUG | | [3132-3150] 3′UTR |
| 120 | GCCACAAAUGUUUAGAACU | AGUUCUAAACAUUUGUGGC | | [3130-3148] 3′UTR |
| 121 | AGCCACAAAUGUUUAGAAC | GUUCUAAACAUUUGUGGCU | | [3129-3147] 3′UTR |
| 122 | GAGCAGGGUUUUCUUUUUG | CAAAAAGAAAACCCUGCUC | | [3050-3068] 3′UTR |
| 123 | CUCAAUAGUAUGUUCUGAA | UUCAGAACAUACUAUUGAG | | [2966-3004] 3′UTR |
| 124 | ACAACCCAGAAACAAAAAU | AUUUUUGUUUCUGGGUUGU | | [2943-2961] 3′UTR |
| 125 | GGAAUUGUGACAAAUAUGU | ACAUAUUUGUCACAAUUCC | | [2796-2814] 3′UTR |
| 126 | GCUAGCCCUUAUGAAUAUU | AAUAUUCAUAAGGGCUAGC | | [2770-2788] 3′UTR |
| 127 | CUCCUUGUGCUAUAAAAUA | UAUUUUAUAGCACAAGGAG | | [2713-2731] 3′UTR |
| 128 | GCUCCUUGUGCUAUAAAAU | AUUUUAUAGCACAAGGAGC | | [2712-2730] 3′UTR |
| 129 | GCCAUCAUUAUUAUCAUAC | GUAUGAUAAUAAUGAUGGC | | [2208-2228] 3′UTR |
| 130 | CCAUCUUACUCCAGGUUUG | CAAACCUGGAGUAAGAUGG | | [2075-2093] 3′UTR |
| 131 | AGGAUCAAAAGGUUCAGUA | UACUGAACCUUUUGAUCCU | | [2019-2037] 3′UTR |
| 132 | GUCGAAAUCUGCUGUCCUU | AAGGACAGCAGAUUUCGAC | | [229-247] ORF |
| 133 | GGGAAAGGAUCAAAAGGUU | AACCUUUUGAUCCUUUCCC | | [2014-2032] 3′UTR |
| 134 | GGAAAAGAUUCUUGGACUC | GAGUCCAAGAAUCUUUUCC | | [1979-1997] 3′UTR |
| 135 | GGGAAAAGAUUCUUGGACU | AGUCCAAGAAUCUUUUCCC | | [1978-1996] 3′UTR |
| 136 | CCUGAGUAAACAAAGCAUC | GAUGCUUUGUUUACUCAGG | | [1649-1667] ORF |
| 137 | UCAGUCUGGUACAAAUAUU | AAUAUUUGUACCAGACUGA | Rabbit | [1278-1298] ORF |
| 138 | UGGGACAAUACAUUUUUGU | ACAAAAAUGUAUUGUCCCA | | [976-994] ORF |
| 139 | GUGGGACAAUACAUUUUUG | CAAAAAUGUAUUGUCCCAC | | [975-993] ORF |
| 140 | AGUGGGACAAUACAUUUUU | AAAAAUGUAUUGUCCCACU | | [974-992] ORF |
| 141 | CCGGGUUUAUGAUAUUCCA | UGGAAUAUCAUAAACCCGG | | [104-122] ORF |
| 142 | GGAAAAAUAAAGGAAUGCC | GGCAUUCCUUUAUUUUUCC | | [768-786] ORF |
| 143 | GGCUCUCCAUUUUUGUCAU | AUGACAAAAAUGGAGAGCC | | [40-58] ORF |
| 144 | ACGCUGUGCCUCAUAUUAA | UUAAUAUGAGGCACAGCGU | | [561-579] ORF |
| 145 | CGAAAGAGAAUAAAGAACC | GGUUCUUUAUUCUCUUUCG | | [483-501] ORF |
| 146 | GAAUGCCCGAGUCAAUAAU | AUUAUUGACUCGGGCAUUC | | [395-413] ORF |
| 147 | CAGGCUUUGUAUGUGAAUA | UAUUCACAUACAAAGCCUG | | [4169-4187] 3′UTR |
| 148 | GGCUCUGUUUAUUGUUUCA | UGAAACAAUAAACAGAGCC | | [3700-3718] 3′UTR |
| 149 | GUUGUUCUUUGUCAUACUA | UAGUAUGACAAAGAACAAC | | [3482-3500] 3′UTR |
| 150 | UCAGUUGUUCUUUGUCAUA | UAUGACAAAGAACAACUGA | | [3479-3497] 3′UTR |
| 151 | CUCAGUUGUUCUUUGUCAU | AUGACAAAGAACAACUGAG | | [3478-3498] 3′UTR |
| 152 | GAAAAGAGAAUGGGAGAGU | ACUCUCCCAUUCUCUUUUC | | [3231-3249] 3′UTR |
| 153 | GAAAAGGAAAUAAGGAGAA | UUCUCCUUAUUUCCUUUUC | | [3215-3233] 3′UTR |
| 154 | CAAAUGUUUAGAACUCUUC | GAAGAGUUCUAAACAUUUG | | [3134-3152] 3′UTR |
| 155 | GAGUCUCUAAAUCACUGUU | AACAGUGAUUUAGAGACUC | | [3020-3038] 3′UTR |
| 156 | GUAUCUGAUAUGGUCAUUU | AAAUGACCAUAUCAGAUAC | | [2813-2831] 3′UTR |
| 157 | AGCAUGAAAAAAGAGGGUU | AACCCUCUUUUUUCAUGCU | | [2645-2683] 3′UTR |
| 158 | ACACAAUGCCAAGAUAGAA | UUCUAUCUUGGCAUUGUGU | | [2476-2494] 3′UTR |
| 159 | CUACAGAAGAAUAUUGGAU | AUCCAAUAUUCUUCUGUAG | | [2359-2377] 3′UTR |
| 160 | ACCAAGGAGUAGCUAUAUA | UAUAUAGCUACUCCUUGGU | | [2168-2188] 3′UTR |
| 161 | CUGCCUCAGAAUCCUCAAA | UUUGAGGAUUCUGAGGCAG | | [2116-2134] 3′UTR |
| 162 | CCACAAUACUGAGCUGCCU | AGGCAGCUCAGUAUUGUGG | | [2103-2121] 3′UTR |
| 163 | UUGAUACUCUUUCCACAAU | AUUGUGGAAAGAGUAUCAA | | [2091-2109] 3′UTR |
| 164 | GUAAUGGUUUUCCCUUAAA | UUUAAGGGAAAACCAUUAC | | [1840-1858] 3′UTR |
| 165 | CCAUGAGGAAAUAAAUGUG | CACAUUUAUUUCCUCAUGG | | [1738-1756] 3′UTR |
| 166 | GGAGUGCAUUUCAUUUUCA | UGAAAAUGAAAUGCACUCC | | [1692-1710] ORF |
| 167 | GUAAACAAAGCAUCUCCAA | UUGGAGAUGCUUUGUUUAC | | [1654-1672] ORF |
| 168 | GGAGAAAGAUGUGAUCACA | UGUGAUCACAUCUUUCUCC | Rabbit, Ms | [1505-1523] ORF |
| 169 | CCAUUCUCAAGUCAGUCUG | CAGACUGACUUGAGAAUGG | | [1267-1285] ORF |
| 170 | UCAAGAUGCGUGGAAACUA | UAGUUUCCACGCAUCUUGA | | [1142-1180] ORF |
| 171 | AGUUUCAAGAUGCGUGGAA | UUCCACGCAUCUUGAAACU | | [1138-1156] ORF |
| 172 | GGAGUUUCAAGAUGCGUGG | CCACGCAUCUUGAAACUCC | | [1136-1154] ORF |
| 173 | AGACUUCUUUAGUAUCCAU | AUGGAUACUAAAGAAGUCU | | [1058-1076] ORF |
| 174 | GAGGAAGACUUCUUUAGUA | UACUAAAGAAGUCUUCCUC | | [1053-1071] ORF |
| 175 | AAACCAUCGAGCUACAGAU | AUCUGUAGCUCGAUGGUUU | | [931-949] ORF |
| 176 | AGAGGUUGGUGCGGUUUUG | CAAAACCGCACCAACCUCU | | [682-860] ORF |
| 177 | GAGAGGUUGGUGCGGUUUU | AAAACCGCACCAACCUCUC | | [861-879] ORF |
| 178 | GCUCUCCAUUUUUGUCAUU | AAUGACAAAAAUGGAGAGC | | [41-59] ORF |
| 179 | GUGAACAAAAAAUCUCAGA | UCUGAGAUUUUUUGUUCAC | | [745-763] ORF |
| 180 | CACGCUGUGCCUCAUAUUA | UAAUAUGAGGCACAGCGUG | | [580-578] ORF |
| 181 | GCCCGAGUCAAUAAUUCUG | CAGAAUUAUUGACUCGGGC | | [399-417] ORF |
| 182 | GUGCUCAACUAUUAAAAUG | CAUUUUAAUAGUUGAGCAC | | [4237-4255] 3′UTR |
| 183 | ACUGUGCUCAACUAUUAAA | UUUAAUAGUUGAGCACAGU | | [4234-4252] 3′UTR |
| 184 | UGCAGGCUUUGUAUGUGAA | UUCACAUACAAAGCCUGCA | | [4167-4185] 3′UTR |
| 185 | GGUUUUUCUUAGUUCUUCU | AGAAGAACUAAGAAAAACC | | [4114-4132] 3′UTR |
| 186 | AGGUUUUUCUUAGUUCUUC | GAAGAACUAAGAAAAACCU | | [4113-4131] 3′UTR |
| 187 | GAAGGUUUUUCUUAGUUCU | AGAACUAAGAAAAACCUUC | | [4111-4129] 3′UTR |
| 188 | GAAUGAUGAAGCAUUGUUU | AAACAAUGCUUCAUCAUUC | | [4076-4094] 3′UTR |
| 189 | AGAAUGAUGAAGCAUUGUU | AACAAUGCUUCAUCAUUCU | | [4075-4093] 3′UTR |
| 190 | CUAAGAAUGAUGAAGCAUU | AAUGCUUCAUCAUUCUUAG | | [4072-4090] 3′UTR |
| 191 | AUAUAAGAGUGCUUGUCAU | AUGACAAGCACUCUUAUAU | | [3929-3947] 3′UTR |
| 192 | GAAUAUAAGAGUGCUUGUC | GACAAGCACUCUUAUAUUC | | [3927-3945] 3′UTR |
| 193 | CCCUUUCUAACCUAUGUGC | GCACAUAGGUUAGAAAGGG | | [3783-3801] 3′UTR |
| 194 | AUAUAGCCUUUCUGAAAUG | CAUUUCAGAAAGGCUAUAU | | [3571-3589] 3′UTR |
| 195 | AGGCCUGCUGUGCGAAUAU | AUAUUCGCACAGCAGGCCU | | [3556-3574] 3′UTR |
| 196 | CUAGGAUGAACUAAAUAAG | CUUAUUUAGUUCAUCCUAG | | [3457-3475] 3′UTR |
| 197 | GGAUUAUUCAAGGGACUAG | CUAGUCCCUUGAAUAAUCC | | [3442-3460] 3′UTR |
| 198 | GGAGAGUGAGAGAAAAUAA | UUAUUUUCUCUCACUCUCC | | [3243-3261] 3′UTR |
| 199 | GGGAGAGUGAGAGAAAAUA | UAUUUUCUCUCACUCUCCC | | [3242-3260] 3′UTR |
| 200 | AGAAAAGAGAAUGGGAGAG | CUCUCCCAUUCUCUUUUCU | | [3230-3248] 3′UTR |
| 201 | GGAAAUAAGGAGAAAAGAG | CUCUUUUCUCCUUAUUUCC | | [3220-3238] 3′UTR |
| 202 | GGAAAAGGAAAUAAGGAGA | UCUCCUUAUUUCCUUUUCC | | [3214-3232] 3′UTR |
| 203 | GAACUCUUCAACUUCGGUA | UACCGAAGUUGAAGAGUUC | | [3144-3162] 3′UTR |
| 204 | UGGAGCCACAAAUGUUUAG | CUAAACAUUUGUGGCUCCA | | [3126-3144] 3′UTR |
| 205 | GGUUGAACGUCUUCCUCUU | AAGAGGAAGACGUUCAACC | | [73-91] ORF |
| 206 | CAAGAGCAGGGUUUUCUUU | AAAGAAAACCCUGCUCUUG | | [3047-3065] 3′UTR |
| 207 | AGUCUCUAAAUCACUGUUA | UAACAGUGAUUUAGAGACU | | [3021-3039] 3′UTR |
| 208 | AGAGUCUCUAAAUCACUGU | ACAGUGAUUUAGAGACUCU | | [3019-3037] 3′UTR |
| 209 | UAACAACCCAGAAACAAAA | UUUUGUUUCUGGGUUGUUA | | [2941-2959] 3′UTR |
| 210 | CUAAAAUUAACAACCCAGA | UCUGGGUUGUUAAUUUUAG | | [2934-2952] 3′UTR |
| 211 | UCCUAAAAUUAACAACCCA | UGGGUUGUUAAUUUUAGGA | | [2932-2950] 3′UTR |
| 212 | AGCCCUUAUGAAUAUUGAA | UUCAAUAUUCAUAAGGGCU | | [2773-2791] 3′UTR |
| 213 | CCAGCUAGCCCUUAUGAAU | AUUCAUAAGGGCUAGCUGG | | [2767-2785] 3′UTR |
| 214 | CAAUGCCAAGAUAGAAUUA | UAAUUCUAUCUUGGCAUUG | | [2479-2497] 3′UTR |
| 215 | GGAUUGAUUGGAGUUAAUG | CAUUAACUCCAAUCAAUCC | | [2374-2392] 3′UTR |
| 216 | GAAUCCUCAAAAUCAGUUU | AAACUGAUUUUGAGGAUUC | | [2124-2142] 3′UTR |
| 217 | CAGUGGAAGUUAGGGAAAA | UUUUCCCUAACUUCCACUG | | [1966-1984] 3′UTR |
| 218 | AGAGCACUUUUACAAACAU | AUGUUUGUAAAAGUGCUCU | | [1919-1937] 3′UTR |
| 219 | GGUUUUGAGAGCACUUUUA | UAAAAGUGCUCUCAAAACC | | [1912-1930] 3′UTR |
| 220 | ACUGCAUGCUGAUUCUCUU | AAGAGAAUCAGCAUGCAGU | | [202-220] ORF |
| 221 | UGUGAUCACAGGCCUGAAA | UUUCAGGCCUGUGAUCACA | | [1514-1532] ORF |
| 222 | CCAUGAUGAGGAGAAAGAU | AUCUUUCUCCUCAUCAUGG | Rabbit, Ms | [1496-1514] ORF |
| 223 | UGAAUUUCAACUGCAUGCU | AGCAUGCAGUUGAAAUUCA | Rat | [193-211] ORF |
| 224 | CGCAUCCAUUCUCAAGUCA | UGACUUGAGAAUGGAUGCG | | [1262-1280] ORF |
| 225 | CCCUUCGCAUCCAUUCUCA | UGAGAAUGGAUGCGAAGGG | | [1257-1275] ORF |
| 226 | GUUUCAAGAUGCGUGGAAA | UUUCCACGCAUCUUGAAAC | | [1139-1157] ORF |
| 227 | GCAGGAGUUUCAAGAUGCG | CGCAUCUUGAAACUCCUGC | | [1133-1151] ORF |
| 228 | AGGAAGACUUCUUUAGUAU | AUACUAAAGAAGUCUUCCU | | [1054-1072] ORF |
| 229 | CCAUGUUUCUGUAUCUCUG | CAGAGAUACAGAAACAUGG | | [841-859] ORF |
| 230 | UGGCUGUGCAUAAUAUAAC | GUUAUAUUAUGCACAGCCA | | [721-739] ORF |
| 231 | GGUACACACAUCAUCUCUU | AAGAGAUGAUGUGUGUACC | | [631-649] ORF |
| 232 | AGGUCUUACUUUGAAGUCU | AGACUUCAAAGUAAGACCU | | [609-627] ORF |
| 233 | GGAGUUGUCAUCACGCUGU | ACAGCGUGAUGACAACUCC | | [549-567] ORF |
| 234 | UCGAAAGAGAAUAAAGAAC | GUUCUUUAUUCUCUUUCGA | | [482-500] ORF |
| 235 | GGAGACAGGCAAAAUGAAA | UUUCAUUUUGCCUGUCUCC | | [447-465] ORF |
| 236 | AGUCAAUAAUUCUGAUCCU | AGGAUCAGAAUUAUUGACU | | [404-422] ORF |
| 237 | UGAAUGCCCGAGUCAAUAA | UUAUUGACUCGGGCAUUCA | | [394-412] ORF |
| 238 | GCUUUUGCAAUUGUGUUUG | CAAACACAAUUGCAAAAGC | | [4133-4151] 3′UTR |
| 239 | UGAAUAUAAGAGUGCUUGU | ACAAGCACUCUUAUAUUCA | | [3926-3944] 3′UTR |
| 240 | CUCAGUUAUAAGGAGUCUG | CAGACUCCUUAUAACUGAG | | [3754-3772] 3′UTR |
| 241 | UCACACCAUUGCACAUCUA | UAGAUGUGCAAUGGUGUGA | | [356-374] ORF |
| 242 | GCCUGCUGUGCGAAUAUAG | CUAUAUUCGCACAGCAGGC | | [3556-3575] 3′UTR |
| 243 | AGGGUAGGUCUGACAAAUA | UAUUUGUCAGACCUACCCU | | [3537-3555] 3′UTR |
| 244 | CAGUUGUUCUUUGUCAUAC | GUAUGACAAAGAACAACUG | | [3480-3498] 3′UTR |
| 245 | GACUAGGAUGAACUAAAUA | UAUUUAGUUCAUCCUAGUC | | [3455-3473] 3′UTR |
| 246 | GGGAUUAUUCAAGGGACUA | UAGUCCCUUGAAUAAUCCC | | [3441-3459] 3′UTR |
| 247 | ACAUACUGAUGUUGUUUUG | CAAAACAACAUCAGUAUGU | | [3366-3386] 3′UTR |
| 248 | GAGAGUGAGAGAAAAUAAA | UUUAUUUUCUCUCACUCUC | | [3244-3282] 3′UTR |
| 249 | UGAGGAAGAAGGAGAAAGA | UCUUUCUCCUUCUUCCUCA | | [3164-3182] 3′UTR |
| 250 | GAAUAUAUGUUCAAGAGAG | CUCUCUUGAACAUAUAUUC | | [3002-3020] 3′UTR |
| 251 | CAUCAUCAUCAUCCUAAAA | UUUUAGGAUGAUGAUGAUG | | [2921-2939] 3′UTR |
| 252 | ACUUAGGAAUUGUGACAAA | UUUGUCACAAUUCCUAAGU | | [2791-2809] 3′UTR |
| 253 | CUAGCCCUUAUGAAUAUUG | CAAUAUUCAUAAGGGCUAG | | [2771-2789] 3′UTR |
| 254 | UGAAAAAAGAGGGUUGGAG | CUCCAACCCUCUUUUUUCA | | [2649-2857] 3′UTR |
| 255 | GGGUUCUGCACAUUCUUAU | AUAAGAAUGUGCAGAACCC | | [2622-2640] 3′UTR |
| 256 | UUGGAUUGAUUGGAGUUAA | UUAACUCCAAUCAAUCCAA | | [2372-2390] 3′UTR |
| 257 | GUGUCAUUUUUGCCAUCAU | AUGAUGGCAAAAAUGACAC | | [2197-2215] 3′UTR |
| 258 | AGAAUCCUCAAAAUCAGUU | AACUGAUUUUGAGGAUUCU | | [2123-2141] 3′UTR |
| 259 | AGGUUUGAUACUCUUUCCA | UGGAAAGAGUAUCAAACCU | | [2087-2105] 3′UTR |
| 260 | UCCAGGUUUGAUACUCUUU | AAAGAGUAUCAAACCUGGA | | [2084-2102] 3′UTR |
| 261 | GUAACUUCCCUAAGAUUAU | AUAAUCUUAGGGAAGUUAC | | [2035-2053] 3′UTR |
| 262 | CAAAAGGGAAAGGAUCAAA | UUUGAUCCUUUCCCUUUUG | | [2009-2027] 3′UTR |
| 263 | AAAGAUUCUUGGACUCAAU | AUUGAGUCCAAGAAUCUUU | | [1982-2000] 3′UTR |
| 264 | GGAAGUUAGGGAAAAGAUU | AAUCUUUUCCCUAACUUCC | | [1970-1988] 3′UTR |
| 265 | GAGCACUUUUACAAACAUU | AAUGUUUGUAAAAGUGCUC | | [1920-1938] 3′UTR |
| 266 | GGAAUGUCAAAGAUUGUUU | AAACAAUCUUUGACAUUCC | | [1859-1877] 3′UTR |
| 267 | GAGUGCAUUUCAUUUUCAA | UUGAAAAUGAAAUGCACUC | | [1593-1711] ORF |
| 268 | GGGAGUGCAUUUCAUUUUC | GAAAAUGAAAUGCACUCCC | | [1691-1709] ORF |
| 269 | GACUUCUUUAGUAUCCAUA | UAUGGAUACUAAAGAAGUC | | [1059-1077] ORF |
| 270 | GCACCCUUUUACACUGACA | UGUCAGUGUAAAAGGGUGC | | [1025-1043] ORF |
| 271 | CCUAAGUUCUUUUACACAA | UUGUGUAAAAGAACUUAGG | | [123-141] ORF |
| 272 | AAGUGGGACAAUACAUUUU | AAAAUGUAUUGUCCCACUU | | [973-991] ORF |
| 273 | CAAAAUGGAAGUGGGACAA | UUGUCCCACUUCCAUUUUG | | [965-963] ORF |
| 274 | CUCACCCUUUCAAAACCAU | AUGGUUUUGAAAGGGUGAG | Rabbit | [919-937] ORF |
| 275 | CGGUUUUGGCGAUCUCAAC | GUUGAGAUCGCCAAAACCG | | [873-891] ORF |
| 276 | UGAGAGGUUGGUGCGGUUU | AAACCGCACCAACCUCUCA | | [860-878] ORF |
| 277 | AGAGAGUUUGGCUGUGCAU | AUGCACAGCCAAACUCUCU | | [713-731] ORF |
| 278 | CAGAGAGUUUGGCUGUGCA | UGCACAGCCAAACUCUCUG | | [712-730] ORF |
| 279 | ACACACAUCAUCUCUUUGU | ACAAAGAGAUGAUGUGUGU | | [634-652] ORF |
| 280 | ACUUUGAAGUCUUUUGGUA | UACCAAAAGACUUCAAAGU | | [616-634] ORF |
| 281 | UCACUGGAGUUGUCAUCAC | GUGAUGACAACUCCAGUGA | | [544-562] ORF |
| 282 | UCCUCUUUGUCUGGUAUUA | UAAUACCAGACAAAGAGGA | | [85-103] ORF |
| 283 | GACAGGCAAAAUGAAAGUU | AACUUUCAUUUUGCCUGUC | | [450-468] ORF |
| 284 | UGCAAUUGUGUUUGUGAAA | UUUCACAAACACAAUUGCA | | [4138-4156] 3′UTR |
| 285 | UUGCAAUUGUGUUUGUGAA | UUCACAAACACAAUUGCAA | | [4137-4155] 3′UTR |
| 286 | CUUUUGCAAUUGUGUUUGU | ACAAACACAAUUGCAAAAG | | [4134-4152] 3′UTR |
| 287 | CUUAAGACUAUGAAGGUUU | AAACCUUCAUAGUCUUAAG | | [4100-4118] 3′UTR |
| 288 | AGCAUUUAUUAACAGGUAC | GUACCUGUUAAUAAAUGCU | | [4052-4070] 3′UTR |
| 289 | AGCACUUAGUAUUUAGCAU | AUGCUAAAUACUAAGUGCU | | [4038-4056] 3′UTR |
| 290 | CCAAGGUUUUCUAUCUUCA | UGAAGAUAGAAAACCUUGG | | [3844-3862] 3′UTR |
| 291 | CUCCAAGGUUUUCUAUCUU | AAGAUAGAAAACCUUGGAG | | [3842-3860] 3′UTR |
| 292 | AGAAUAUUGGCCCUUUCUA | UAGAAAGGGCCAAUAUUCU | | [3773-3791] 3′UTR |
| 293 | ACCAUUGCACAUCUAUUUA | UAAAUAGAUGUGCAAUGGU | | [360-378] ORF |
| 294 | AGUUGUUCUUUGUCAUACU | AGUAUGACAAAGAACAACU | | [3481-3499] 3′UTR |
| 295 | GAAUAUAAUUGAAGGAGGU | ACCUCCUUCAAUUAUAUUC | | [3345-3383] 3′UTR |
| 296 | GAAUGGGAGAGUGAGAGAA | UUCUCUCACUCUCCCAUUC | | [3238-3256] 3′UTR |
| 297 | CUUGGAGCCACAAAUGUUU | AAACAUUUGUGGCUCCAAG | | [3124-3142] 3′UTR |
| 298 | GUCUCUAAAUCACUGUUAG | CUAACAGUGAUUUAGAGAC | | [3022-3040] 3′UTR |
| 299 | GAGAGUCUCUAAAUCACUG | CAGUGAUUUAGAGACUCUC | | [3018-3036] 3′UTR |
| 300 | CAACCCAGAAACAAAAAUC | GAUUUUUGUUUCUGGGUUG | | [2944-2962] 3′UTR |
| 301 | UGUAUCUGAUAUGGUCAUU | AAUGACCAUAUCAGAUACA | | [2812-2830] 3′UTR |
| 302 | CUCAUCUAGUCCUGCUCCU | AGGAGCAGGACUAGAUGAG | | [2699-2717] 3′UTR |
| 303 | UGCACAUUCUUAUUUGAAG | CUUCAAAUAAGAAUGUGCA | | [2628-2646] 3′UTR |
| 304 | CUGCACAUUCUUAUUUGAA | UUCAAAUAAGAAUGUGCAG | | [2627-2645] 3′UTR |
| 305 | CCUGAAGGGUUCUGCACAU | AUGUGCAGAACCCUUCAGG | | [2616-2634] 3′UTR |
| 306 | UACUAAUACAGCAGGGUAA | UUACCCUGCUGUAUUAGUA | | [2322-2340] 3′UTR |
| 307 | CAAGGAGUAGCUAUAUAUU | AAUAUAUAGCUACUCCUUG | | [2170-2188] 3′UTR |
| 308 | UCAGAAUCCUCAAAAUCAG | CUGAUUUUGAGGAUUCUGA | | [2121-2139] 3′UTR |
| 309 | CCCUAAGAUUAUGAAACUG | CAGUUUCAUAAUCUUAGGG | | [2042-2060] 3′UTR |
| 310 | CAGUAACUUCCCUAAGAUU | AAUCUUAGGGAAGUUACUG | | [2033-2051] 3′UTR |
| 311 | AUCAAAAGGUUCAGUAACU | AGUUACUGAACCUUUUGAU | | [2022-2040] 3′UTR |
| 312 | UCAAAAGGGAAAGGAUCAA | UUGAUCCUUUCCCUUUUGA | | [2008-2026] 3′UTR |
| 313 | AAAAGAUUCUUGGACUCAA | UUGAGUCCAAGAAUCUUUU | | [1961-1999] 3′UTR |
| 314 | CUGCUUAAAAAUGGACAAA | UUUGUCCAUUUUUAAGCAG | | [1808-1826] 3′UTR |
| 315 | CCUGCUUAAAAAUGGACAA | UUGUCCAUUUUUAAGCAGG | | [1807-1825] 3′UTR |
| 316 | GAGGAAAUAAAUGUGGGUU | AACCCACAUUUAUUUCCUC | | [1742-1760] 3′UTR |
| 317 | CAACAAGGAAAACUUCUAA | UUAGAAGUUUUCCUUGUUG | Rat, Ms | [1709-1727] ORF |
| 318 | GUGCAUUUCAUUUUCAACA | UGUUGAAAAUGAAAUGCAC | | [1695-1713] ORF |
| 319 | ACAGGCCUGAAACAAAAGA | UCUUUUGUUUCAGGCCUGU | | [1521-1539] ORF |
| 320 | UCCUCAGCUACAACAUCUA | UAGAUGUUGUAGCUGAGGA | Rat, Rabbit | [1432-1450] ORF |
| 321 | AGAUGCAGGAAAGGAACAA | UUGUUCCUUUCCUGCAUCU | Ms | [1405-1423] ORF |
| 322 | CCAGAUGCAGGAAAGGAAC | GUUCCUUUCCUGCAUCUGG | Ms | [1403-1421] ORF |
| 323 | UCGCAUCCAUUCUCAAGUC | GACUUGAGAAUGGAUGCGA | | [1261-1279] ORF |
| 324 | UCAGCUAUGAGGUGGUGAU | AUCACCACCUCAUAGCUGA | Rat, Rabbit, Ms | [1210-1228] ORF |
| 325 | CUCCAUUUUUGUCAUUCUG | CAGAAUGACAAAAAUGGAG | | [44-62] ORF |
| 326 | CACCCUUUUACACUGACAU | AUGUCAGUGUAAAAGGGUG | | [1026-1044] ORF |
| 327 | UUCCACCUAAGUUCUUUUA | UAAAAGAACUUAGGUGGAA | | [118-136] ORF |
| 328 | GUCCCAUGUUUCUGUAUCU | AGAUACAGAAACAUGGGAC | | [838-856] ORF |
| 329 | GGGUCCCAUGUUUCUGUAU | AUACAGAAACAUGGGACCC | | [836-854] ORF |
| 330 | GUUUAUGAUAUUCCACCUA | UAGGUGGAAUAUCAUAAAC | | [108-126] ORF |
| 331 | CGGGUUUAUGAUAUUCCAC | GUGGAAUAUCAUAAACCCG | | [105-123] ORF |
| 332 | AGAGUUUGGCUGUGCAUAA | UUAUGCACAGCCAAACUCU | | [715-733] ORF |
| 333 | UGAAGUCUUUUGGUACACA | UGUGUACCAAAAGACUUCA | | [620-638] ORF |
| 334 | GCAUCACUGGAGUUGUCAU | AUGACAACUCCAGUGAUGC | | [541-559] ORF |
| 335 | AGACAGGCAAAAUGAAAGU | ACUUUCAUUUUGCCUGUCU | | [449-467] ORF |
| 336 | UGAACUUGGAGACAGGCAA | UUGCCUGUCUCCAAGUUCA | | [440-458] ORF |
| 337 | UGAUCCUUAUUCAGUAGCA | UGCUACUGAAUAAGGAUCA | | [416-434] ORF |
| 338 | GGACCUGGGAGAUAAUUCU | AGAAUUAUCUCCCAGGUCC | | [4199-4217] 3′UTR |
| 339 | UCUUAAGACUAUGAAGGUU | AACCUUCAUAGUCUUAAGA | | [4099-4117] 3′UTR |
| 340 | CUCUAAGAAUGAUGAAGCA | UGCUUCAUCAUUCUUAGAG | | [4070-4088] 3′UTR |
| 341 | GUACUCUAAGAAUGAUGAA | UUCAUCAUUCUUAGAGUAC | | [4067-4065] 3′UTR |
| 342 | CAGGUACUCUAAGAAUGAU | AUCAUUCUUAGAGUACCUG | | [4064-4062] 3′UTR |
| 343 | AAAACCAGCUUCAUUUGUU | AACAAAUGAAGCUGGUUUU | | [3808-3826] 3′UTR |
| 344 | GUGUUAUUACUAUGAGUCA | UGACUCAUAGUAAUAACAC | | [3681-3699] 3′UTR |
| 345 | UGCUUAGAGACACUUAGGU | ACCUAAGUGUCUCUAAGCA | | [3605-3623] 3′UTR |
| 346 | GGAUGGUUUCUGCUUAGAG | CUCUAAGCAGAAACCAUCC | | [3595-3613] 3′UTR |
| 347 | UCAUACUACUAUUCCUUUC | GAAAGGAAUAGUAGUAUGA | | [3493-3511] 3′UTR |
| 348 | UGUCAUACUACUAUUCCUU | AAGGAAUAGUAGUAUGACA | | [3491-3509] 3′UTR |
| 349 | GAAGGAGGUAUACACAUAC | GUAUGUGUAUACCUCCUUC | | [3355-3373] 3′UTR |
| 350 | AAGGAAAUAAGGAGAAAAG | CUUUUCUCCUUAUUUCCUU | | [3218-3236] 3′UTR |
| 351 | GACUGGUUUAGGAGGAAAA | UUUUCCUCCUAAACCAGUC | | [3201-3219] 3′UTR |
| 352 | CGGUAAUGAGGAAGAAGGA | UCCUUCUUCCUCAUUACCG | | [3158-3176] 3′UTR |
| 353 | CUCUUCAACUUCGGUAAUG | CAUUACCGAAGUUGAAGAG | | [3147-3165] 3′UTR |
| 354 | CAUGGAUGAUUGCACUUCA | UGAAGUGCAAUCAUCCAUG | | [328-346] ORF |
| 355 | CCUUGGAGCCACAAAUGUU | AACAUUUGUGGCUCCAAGG | | [3123-3141] 3′UTR |
| 356 | CCCUUGGAGCCACAAAUGU | ACAUUUGUGGCUCCAAGGG | | [3122-3140] 3′UTR |
| 357 | CAAAUUCACACUCAAUAGU | ACUAUUGAGUGUGAAUUUG | | [2976-2994] 3′UTR |
| 358 | AUGUAUCUGAUAUGGUCAU | AUGACCAUAUCAGAUACAU | | [2811-2829] 3′UTR |
| 359 | CUUAGGAAUUGUGACAAAU | AUUUGUCACAAUUCCUAAG | | [2792-2810] 3′UTR |
| 360 | UCAUCUAGUCCUGCUCCUU | AAGGAGCAGGACUAGAUGA | | [2700-2718] 3′UTR |
| 361 | GGGUUGGAGGUGGAGAAUU | AAUUCUCCACCUCCAACCC | | [2659-2677] 3′UTR |
| 362 | CAUUCUUAUUUGAAGCAUG | CAUGCUUCAAAUAAGAAUG | | [2632-2650] 3′UTR |
| 363 | GUAAUACUCAUCAUUUACC | GGUAAAUGAUGAGUAUUAC | | [2392-2410] 3′UTR |
| 364 | AAAAGAAGAAGGAAACCAA | UUGGUUUCCUUCUUCUUUU | | [2154-2172] 3′UTR |
| 365 | CAAAAGAAGAAGGAAACCA | UGGUUUCCUUCUUCUUUUG | | [2153-2171] 3′UTR |
| 366 | GCUGCCUCAGAAUCCUCAA | UUGAGGAUUCUGAGGCAGC | | [2115-2133] 3′UTR |
| 367 | CAGGUUUGAUACUCUUUCC | GGAAAGAGUAUCAAACCUG | | [2086-2104] 3′UTR |
| 368 | UCUUACUCCAGGUUUGAUA | UAUCAAACCUGGAGUAAGA | | [2078-2096] 3′UTR |
| 369 | CCUAAGAUUAUGAAACUGU | ACAGUUUCAUAAUCUUAGG | | [2043-2061] 3′UTR |
| 370 | UCCCUAAGAUUAUGAAACU | AGUUUCAUAAUCUUAGGGA | | [2041-2059] 3′UTR |
| 371 | AAAAGGGAAAGGAUCAAAA | UUUUGAUCCUUUCCCUUUU | | [2010-2028] 3′UTR |
| 372 | AGGGAAAAGAUUCUUGGAC | GUCCAAGAAUCUUUUCCCU | | [1977-1995] 3′UTR |
| 373 | UCAGUGGAAGUUAGGGAAA | UUUCCCUAACUUCCACUGA | | [1965-1983] 3′UTR |
| 374 | CUAUGGUUUUGAGAGCACU | AGUGCUCUCAAAACCAUAG | | [1908-1926] 3′UTR |
| 375 | UGGAGCUCUAUGGUUUUGA | UCAAAACCAUAGAGCUCCA | | [1901-1919] 3′UTR |
| 376 | GUGGAGCUCUAUGGUUUUG | CAAAACCAUAGAGCUCCAC | | [1900-1918] 3′UTR |
| 377 | CUCUUGCCAGUCUGUCGAA | UUCGACAGACUGGCAAGAG | | [216-234] ORF |
| 378 | AGGAAAUAAAUGUGGGUUG | CAACCCACAUUUAUUUCCU | | [1743-1761] 3′UTR |
| 379 | UGAUUCUCUUGCCAGUCUG | CAGACUGGCAAGAGAAUCA | | [211-229] ORF |
| 380 | AAAGGAACAAUGCCGGCUU | AAGCCGGCAUUGUUCCUUU | | [1414-1432] ORF |
| 381 | GUCAGUCUGGUACAAAUAU | AUAUUUGUACCAGACUGAC | Rabbit | [1277-1295] ORF |
| 382 | CAAGUCAGUCUGGUACAAA | UUUGUACCAGACUGACUUG | Rabbit | [1274-1292] ORF |
| 383 | GGAAGACUUCUUUAGUAUC | GAUACUAAAGAAGUCUUCC | | [1055-1073] ORF |
| 384 | CCUGAGGAAGACUUCUUUA | UAAAGAAGUCUUCCUCAGG | Rat | [1050-1068] ORF |
| 385 | GGCACCCUUUUACACUGAC | GUCAGUGUAAAAGGGUGCC | | [1024-1042] ORF |
| 386 | CACCUAAGUUCUUUUACAC | GUGUAAAAGAACUUAGGUG | | [121-139] ORF |
| 387 | AGCUACAGAUGAAGAAGAA | UUCUUCUUCAUCUGUAGCU | | [940-958] ORF |
| 388 | CUCAACAGAAGGUGGUCAU | AUGACCACCUUCUGUUGAG | | [886-904] ORF |
| 389 | GGCGAUCUCAACAGAAGGU | ACCUUCUGUUGAGAUCGCC | | [880-898] ORF |
| 390 | UGAUAUUCCACCUAAGUUC | GAACUUAGGUGGAAUAUCA | | [113-131] ORF |
| 391 | CCCAUGUUUCUGUAUCUCU | AGAGAUACAGAAACAUGGG | | [840-858] ORF |
| 392 | GGUCCCAUGUUUCUGUAUC | GAUACAGAAACAUGGGACC | | [837-855] ORF |
| 393 | GUUUGGCUGUGCAUAAUAU | AUAUUAUGCACAGCCAAAC | | [718-736] ORF |
| 394 | UCUUUGUGAUCUUCUUCAU | AUGAAGAAGAUCACAAAGA | Rat, Ms | [646-664] ORF |
| 395 | AUCUCUUUGUGAUCUUCUU | AAGAAGAUCACAAAGAGAU | Rat, Ms | [643-661] ORF |
| 396 | UCAUCUCUUUGUGAUCUUC | GAAGAUCACAAAGAGAUGA | | [641-659] ORF |
| 397 | GAAGUCUUUUGGUACACAC | GUGUGUACCAAAAGACUUC | | [621-639] ORF |
| 398 | AGGCAAAAUGAAAGUUAUG | GAUAACUUUCAUUUUGCCU | | [453-471] ORF |
| 399 | GGGCUCUCCAUUUUUGUCA | UGACAAAAAUGGAGAGCCC | | [39-57] ORF |
| 400 | GGUACUCUAAGAAUGAUGA | UCAUCAUUCUUAGAGUACC | | [4066-4084] 3′UTR |
| 401 | CAAGGUUUUCUAUCUUCAA | UUGAAGAUAGAAAACCUUG | | [3845-3863] 3′UTR |
| 402 | CUAUUUAAUGUGGAAUGGU | ACCAUUCCACAUUAAAUAG | | [372-390] ORF |
| 403 | AGGCUCUGUUUAUUGUUUC | GAAACAAUAAACAGAGCCU | | [3699-3717] 3′UTR |
| 404 | ACACCAUUGCACAUCUAUU | AAUAGAUGUGCAAUGGUGU | | [358-378] ORF |
| 405 | AGGAUGGUUUCUGCUUAGA | UCUAAGCAGAAACCAUCCU | | [3594-3612] 3′UTR |
| 406 | UGAGGGUAGGUCUGACAAA | UUUGUCAGACCUACCCUCA | | [3535-3553] 3′UTR |
| 407 | GUCAUACUACUAUUCCUUU | AAAGGAAUAGUAGUAUGAC | | [3492-3510] 3′UTR |
| 408 | ACUCAGUUGUUCUUUGUCA | UGACAAAGAACAACUGAGU | | [3477-3495] 3′UTR |
| 409 | CUGGUCACAAAUUUUGAUG | CAUCAAAAUUUGUGACCAG | | [3416-3434] 3′UTR |
| 410 | UCUGGUCACAAAUUUUGAU | AUCAAAAUUUGUGACCAGA | | [3415-3433] 3′UTR |
| 411 | AGACUGGUUUAGGAGGAAA | UUUCCUCCUAAACCAGUCU | | [3200-3218] 3′UTR |
| 412 | UAAUGAGGAAGAAGGAGAA | UUCUCCUUCUUCCUCAUUA | | [3161-3179] 3′UTR |
| 413 | GUAAUGAGGAAGAAGGAGA | UCUCCUUCUUCCUCAUUAC | | [3160-3178] 3′UTR |
| 414 | CUUCGGUAAUGAGGAAGAA | UUCUUCCUCAUUACCGAAG | | [3155-3173] 3′UTR |
| 415 | AACUCUUCAACUUCGGUAA | UUACCGAAGUUGAAGAGUU | | [3145-3163] 3′UTR |
| 416 | ACAGAGAUCAAAUUCACAC | GUGUGAAUUUGAUCUCUGU | | [2968-2986] 3′UTR |
| 417 | CUGAAGGGUUCUGCACAUU | AAUGUGCAGAACCCUUCAG | | [2617-2835] 3′UTR |
| 418 | AGGGUCAAGAACAGGCUAA | UUAGCCUGUUCUUGACCCU | | [2576-2594] 3′UTR |
| 419 | UCAUUUACCACUGUGCUUG | CAAGCACAGUGGUAAAUGA | | [2402-2420] 3′UTR |
| 420 | CUCAUCAUUUACCACUGUG | CACAGUGGUAAAUGAUGAG | | [2396-2416] 3′UTR |
| 421 | CAGAAGAAUAUUGGAUUGA | UCAAUCCAAUAUUCUUCUG | | [2362-2380] 3′UTR |
| 422 | CCUCAGAAUCCUCAAAAUC | GAUUUUGAGGAUUCUGAGG | | [2119-2137] 3′UTR |
| 423 | CUAAGAUUAUGAAACUGUG | CACAGUUUCAUAAUCUUAG | | [2044-2062] 3′UTR |
| 424 | AGAUUCUUGGACUCAAUUU | AAAUUGAGUCCAAGAAUCU | | [1964-2002] 3′UTR |
| 425 | GUCAGUGGAAGUUAGGGAA | UUCCCUAACUUCCACUGAC | | [1964-1982] 3′UTR |
| 426 | CCCUGCUUAAAAAUGGACA | UGUCCAUUUUUAAGCAGGG | | [1808-1824] 3′UTR |
| 427 | UGAGGAAAUAAAUGUGGGU | ACCCACAUUUAUUUCCUCA | | [1741-1759] 3′UTR |
| 428 | UCUAACUUGUCUCUUCCAU | AUGGAAGAGACAAGUUAGA | | [1723-1741] ORF + 3′UTR |
| 429 | CAAGGAAAACUUCUAACUU | AAGUUAGAAGUUUUCCUUG | | [1712-1730] ORF + 3′UTR |
| 430 | ACAAGGAAAACUUCUAACU | AGUUAGAAGUUUUCCUUGU | Ms | [1711-1729] ORF + 3′UTR |
| 431 | UCAACAAGGAAAACUUCUA | UAGAAGUUUUCCUUGUUGA | Rat, Ms | [1708-1726] ORF |
| 432 | UGAGGAGAAAGAUGUGAUC | GAUCACAUCUUUCUCCUCA | Rabbit, Ms | [1502-1520] ORF |
| 433 | UCAGCUACAACAUCUACCU | AGGUAGAUGUUGUAGCUGA | Rat | [1435-1453] ORF |
| 434 | GCCUUUGAGUGGUUUGCAG | CUGCAAACCACUCAAAGGC | Rabbit, Ms | [1362-1380] ORF |
| 435 | ACAUGCCUUUGAGUGGUUU | AAACCACUCAAAGGCAUGU | Rabbit | [1358-1376] ORF |
| 436 | AAGUCAGUCUGGUACAAAU | AUUUGUACCAGACUGACUU | Rabbit | [1275-1293] ORF |
| 437 | UCCAUUCUCAAGUCAGUCU | AGACUGACUUGAGAAUGGA | | [1266-1284] ORF |
| 438 | ACCCUUCGCAUCCAUUCUC | GAGAAUGGAUGCGAAGGGU | | [1256-1274] ORF |
| 439 | CUGCCAGUGAAGAUGUGUU | AACACAUCUUCACUGGCAG | | [1192-1210] ORF |
| 440 | UGGAAGUGGGACAAUACAU | AUGUAUUGUCCCACUUCCA | | [970-988] ORF |
| 441 | UCAAAAUGGAAGUGGGACA | UGUCCCACUUCCAUUUUGA | | [964-982] ORF |
| 442 | GGGUUCAAAAUGGAAGUGG | CCACUUCCAUUUUGAACCC | | [960-978] ORF |
| 443 | GUCACUCACCCUUUCAAAA | UUUUGAAAGGGUGAGUGAC | Rabbit | [915-933] ORF |
| 444 | UCCCAUGUUUCUGUAUCUC | GAGAUACAGAAACAUGGGA | | [839-857] ORF |
| 445 | UGGGUCCCAUGUUUCUGUA | UACAGAAACAUGGGACCCA | | [835-853] ORF |
| 446 | CCUAUGACUUGGAAAUGGA | UCCAUUUCCAAGUCAUAGG | Rat, Ms | [813-831] ORF |
| 447 | CUCUUUGUGAUCUUCUUCA | UGAAGAAGAUCACAAAGAG | Rat, Ms | [645-663] ORF |
| 448 | GUCUUUUGGUACACACAUC | GAUGUGUGUACCAAAAGAC | | [624-642] ORF |
| 449 | CUGGAGUUGUCAUCACGCU | AGCGUGAUGACAACUCCAG | | [547-565] ORF |
| 450 | AGCACUCUCUGAACUUGGA | UCCAAGUUCAGAGAGUGCU | | [431-449] ORF |
| 451 | UUAACAGGUACUCUAAGAA | UUCUUAGAGUACCUGUUAA | | [4060-4078] 3′UTR |
| 452 | GUGUUAUCCAGUUAUGAGA | UCUCAUAACUGGAUAACAC | | [3900-3918] 3′UTR |
| 453 | GAGAAUAUUGGCCCUUUCU | AGAAAGGGCCAAUAUUCUC | | [3772-3790] 3′UTR |
| 454 | CAAAGUAUGAACUGGAGAG | CUCUCCAGUUCAUACUUUG | | [3730-3748] 3′UTR |
| 455 | AGGGACUAGGAUGAACUAA | UUAGUUCAUCCUAGUCCCU | | [3452-3470] 3′UTR |
| 456 | CAAGGGACUAGGAUGAACU | AGUUCAUCCUAGUCCCUUG | | [3450-3468] 3′UTR |
| 457 | CUUUUAAAAUCUGGUCACA | UGUGACCAGAUUUUAAAAG | | [3406-3424] 3′UTR |
| 458 | UCUUUUAAAAUCUGGUCAC | GUGACCAGAUUUUAAAAGA | | [3405-3423] 3′UTR |
| 459 | AGAAUAUAAUUGAAGGAGG | CCUCCUUCAAUUAUAUUCU | | [3344-3362] 3′UTR |
| 460 | UGGGAGAGUGAGAGAAAAU | AUUUUCUCUCACUCUCCCA | | [3241-3259] 3′UTR |
| 461 | ACUCUUCAACUUCGGUAAU | AUUACCGAAGUUGAAGAGU | | [3146-3164] 3′UTR |
| 462 | GGAACUAAAACCAGUUUUA | UAAAACUGGUUUUAGUUCC | | [3093-3111] 3′UTR |
| 463 | CAGAGAUCAAAUUCACACU | AGUGUGAAUUUGAUCUCUG | | [2969-2987] 3′UTR |
| 464 | UCUGAUAUGGUCAUUUGUU | AACAAAUGACCAUAUCAGA | | [2816-2834] 3′UTR |
| 465 | AACUUAGGAAUUGUGACAA | UUGUCACAAUUCCUAAGUU | | [2790-2808] 3′UTR |
| 466 | GGGUCAAGAACAGGCUAAG | CUUAGCCUGUUCUUGACCC | | [2577-2595] 3′UTR |
| 467 | ACUCAUCAUUUACCACUGU | ACAGUGGUAAAUGAUGAGU | | [2397-2415] 3′UTR |
| 468 | GAAUAUUGGAUUGAUUGGA | UCCAAUCAAUCCAAUAUUC | | [2367-2385] 3′UTR |
| 469 | UGGGCCAGAUGUUCUUUCU | AGAAAGAACAUCUGGCCCA | | [2342-2360] 3′UTR |
| 470 | AACAAGAGUUCGAAGACAA | UUGUCUUCGAACUCUUGUU | | [275-293] ORF |
| 471 | AGUAUUGACAUCUGAGCAU | AUGCUCAGAUGUCAAUACU | | [2294-2312] 3′UTR |
| 472 | UGAAGGAAAUUUUCCAGAU | AUCUGGAAAAUUUCCUUCA | | [2227-2245] 3′UTR |
| 473 | UGUCAUUUUUGCCAUCAUU | AAUGAUGGCAAAAAUGACA | | [2198-2216] 3′UTR |
| 474 | GAAAGGAUCAAAAGGUUCA | UGAACCUUUUGAUCCUUUC | | [2016-2034] 3′UTR |
| 475 | AGGGAAAGGAUCAAAAGGU | ACCUUUUGAUCCUUUCCCU | | [2013-2031] 3′UTR |
| 476 | GAAUCAAAAGGGAAAGGAU | AUCCUUUCCCUUUUGAUUC | | [2005-2023] 3′UTR |
| 477 | AGAAUCAAAAGGGAAAGGA | UCCUUUCCCUUUUGAUUCU | | [2004-2022] 3′UTR |
| 478 | CUUGGACUCAAUUUUAGAA | UUCUAAAAUUGAGUCCAAG | | [1989-2007] 3′UTR |
| 479 | GAUUCUUGGACUCAAUUUU | AAAAUUGAGUCCAAGAAUC | | [1985-2003] 3′UTR |
| 480 | AGUUAGGGAAAAGAUUCUU | AAGAAUCUUUUCCCUAACU | | [1973-1991] 3′UTR |
| 481 | UGGAAGUUAGGGAAAAGAU | AUCUUUUCCCUAACUUCCA | | [1969-1987] 3′UTR |
| 482 | GUGGAAGUUAGGGAAAAGA | UCUUUUCCCUAACUUCCAC | | [1968-1986] 3′UTR |
| 483 | CUCAGUAAUGUCAGUGGAA | UUCCACUGACAUUAGUGAG | | [1955-1973] 3′UTR |
| 484 | GAGAGCACUUUUACAAACA | UGUUUGUAAAAGUGCUCUC | | [1918-1936] 3′UTR |
| 485 | UGAGAGCACUUUUACAAAC | GUUUGUAAAAGUGCUCUCA | | [1917-1935] 3′UTR |
| 486 | UGGUUUUGAGAGCACUUUU | AAAAGUGCUCUCAAAACCA | | [1911-1929] 3′UTR |
| 487 | AUGGUUUUGAGAGCACUUU | AAAGUGCUCUCAAAACCAU | | [1910-1928] 3′UTR |
| 488 | GGAAAACUUCUAACUUGUC | GACAAGUUAGAAGUUUUCC | | [1715-1733] ORF + UTR |
| 489 | AGGAGAAAGAUGUGAUCAC | GUGAUCACAUCUUUCUCCU | Rabbit, Ms | [1504-1522] ORF |
| 490 | CCAAUCUGAAGCUCAAAAA | UUUUUGAGCUUCAGAUUGG | | [1309-1327] ORF |
| 491 | CAGUCUGGUACAAAUAUUG | CAAUAUUUGUACCAGACUG | Rabbit | [1279-1297] ORF |
| 492 | GCAUCCAUUCUCAAGUCAG | CUGACUUGAGAAUGGAUGC | | [1263-1281] ORF |
| 493 | UGAAGAUGUGUUCAGCUAU | AUAGCUGAACACAUCUUCA | Rat, Rabbit, Ms | [1199-1217] ORF |
| 494 | CUGAGGAAGACUUCUUUAG | CUAAAGAAGUCUUCCUCAG | Rat | [1051-1069] ORF |
| 495 | UCUCCAUUUUUGUCAUUCU | AGAAUGACAAAAAUGGAGA | | [43-61] ORF |
| 496 | CUCUCCAUUUUUGUCAUUC | GAAUGACAAAAAUGGAGAG | | [42-80] ORF |
| 497 | GCGAUCUCAACAGAAGGUG | CACCUUCUGUUGAGAUCGC | | [881-899] ORF |
| 498 | UUAUGAUAUUCCACCUAAG | CUUAGGUGGAAUAUCAUAA | | [110-128] ORF |
| 499 | UGAUCUUCUUCAUUGGCCU | AGGCCAAUGAAGAAGAUCA | | [652-670] ORF |
| 500 | GAGUUGUCAUCACGCUGUG | CACAGCGUGAUGACAACUC | | [550-568] ORF |
| 501 | AGGCAUCACUGGAGUUGUC | GACAACUCCAGUGAUGCCU | | [539-557] ORF |
| 502 | UCCUUAUUCAGUAGCACUC | GAGUGCUACUGAAUAAGGA | | [419-437] ORF |
| 503 | UAAGAAUGAUGAAGCAUUG | CAAUGCUUCAUCAUUCUUA | | [4073-4091] 3′UTR |
| 504 | AGGUACUGUAAGAAUGAUG | CAUCAUUCUUAGAGUACCU | | [4065-4083] 3′UTR |
| 505 | UCCAAGGUUUUCUAUCUUC | GAAGAUAGAAAACCUUGGA | | [3843-3861] 3′UTR |
| 506 | CACACCAUUGCACAUCUAU | AUAGAUGUGCAAUGGUGUG | | [357-375] ORF |
| 507 | CCUUUCGUCUCCCAGAAUC | GAUUCUGGGAGACGAAAGG | | [3508-3524] 3′UTR |
| 508 | UCAAGGGACUAGGAUGAAC | GUUCAUCCUAGUCCCUUGA | | [3449-3467] 3′UTR |
| 509 | AUGGGAGAGUGAGAGAAAA | UUUUCUCUCACUCUCCCAU | | [3240-3258] 3′UTR |
| 510 | AGGAAAAGGAAAUAAGGAG | CUCCUUAUUUCCUUUUCCU | | [3213-3231] 3′UTR |
| 511 | AGGAGGAAAAGGAAAUAAG | CUUAUUUCCUUUUCCUCCU | | [3210-3228] 3′UTR |
| 512 | CUGUUAGUGUGGCCAAGAG | CUCUUGGCCACACUAACAG | | [3034-3052] 3′UTR |
| 513 | UCCAGCUAGCCCUUAUGAA | UUCAUAAGGGCUAGCUGGA | | [2766-2784] 3′UTR |
| 514 | UCUGCACAUUCUUAUUUGA | UCAAAUAAGAAUGUGCAGA | | [2626-2644] 3′UTR |
| 515 | GAACAGGCUAAGGAUACUA | UAGUAUCCUUAGCCUGUUC | | [2584-2602] 3′UTR |
| 516 | AGGAAAUUUUCCAGAUCAU | AUGAUCUGGAAAAUUUCCU | | [2230-2248] 3′UTR |
| 517 | GAAGGAAAUUUUCCAGAUC | GAUCUGGAAAAUUUCCUUC | | [2228-2246] 3′UTR |
| 518 | AGAAGAAGGAAACCAAGGA | UCCUUGGUUUCCUUCUUCU | | [2157-2175] 3′UTR |
| 519 | ACUUCCCUAAGAUUAUGAA | UUCAUAAUCUUAGGGAAGU | | [2038-2056] 3′UTR |
| 520 | GGAAAGGAUCAAAAGGUUC | GAACCUUUUGAUCCUUUCC | | [2015-2033] 3′UTR |
| 521 | GUAAUGUCAGUGGAAGUUA | UAACUUCCACUGACAUUAC | | [1959-1977] 3′UTR |
| 522 | UCAGUAAUGUCAGUGGAAG | CUUCCACUGACAUUACUGA | | [1956-1974] 3′UTR |
| 523 | CAAAGAUUGUUUGAUAGUG | CACUAUCAAACAAUCUUUG | | [1856-1884] 3′UTR |
| 524 | AGGAAUGUCAAAGAUUGUU | AACAAUCUUUGACAUUCCU | | [1858-1876] 3′UTR |
| 525 | GAAAUAAAUGUGGGUUGUG | CACAACCCACAUUUAUUUC | | [1745-1763] 3′UTR |
| 526 | GAAAACUUCUAACUUGUCU | AGACAAGUUAGAAGUUUUC | | [1716-1734] ORF + 3′UTR |
| 527 | CUGAAACAAAAGACUUUGU | ACAAAGUCUUUUGUUUCAG | | [1527-1545] ORF |
| 528 | CCUGAAACAAAAGACUUUG | CAAAGUCUUUUGUUUCAGG | | [1526-1544] ORF |
| 529 | GCUACAACAUCUACCUCAC | GUGAGGUAGAUGUUGUAGC | | [1438-1458] ORF |
| 530 | GAUGCAGGAAAGGAACAAU | AUUGUUCCUUUCCUGCAUC | Ms | [1406-1424] ORF |
| 531 | CCUUCGCAUCCAUUCUCAA | UUGAGAAUGGAUGCGAAGG | | [1256-1276] ORF |
| 532 | UGAUAAGCAGGAGUUUCAA | UUGAAACUCCUGCUUAUCA | Rat, Rabbit | [1127-1145] ORF |
| 533 | GUAUCCAUAUCCGCAUCGU | ACGAUGCGGAUAUGGAUAC | | [1069-1087] ORF |
| 534 | UGAGGAAGACUUCUUUAGU | ACUAAAGAAGUCUUCCUCA | | [1052-1070] ORF |
| 535 | CCCUGAGGAAGACUUCUUU | AAAGAAGUCUUCCUCAGGG | Rat | [1049-1067] ORF |
| 536 | UGGCACCCUUUUACACUGA | UCAGUGUAAAAGGGUGCCA | | [1023-1041] ORF |
| 537 | UGACUUGGAAAUGGAUAGU | ACUAUCCAUUUCCAAGUCA | Ms | [817-835] ORF |
| 538 | CUAUGACUUGGAAAUGGAU | AUCCAUUUCCAAGUCAUAG | Rat, Ms | [814-832] ORF |
| 539 | UGGAGACAGGCAAAAUGAA | UUCAUUUUGCCUGUCUCCA | | [446-484] ORF |
| 540 | CUUCCUCUUUGUCUGGUAU | AUACCAGACAAAGAGGAAG | | [83-101] ORF |
| 541 | UCUUCCUCUUUGUCUGGUA | UACCAGACAAAGAGGAAGA | | [82-100] ORF |
| 542 | GUAGCACUCUCUGAACUUG | CAAGUUCAGAGAGUGCUAC | | [429-447] ORF |
| 543 | ACGUCUUCCUCUUUGUCUG | CAGACAAAGAGGAAGACGU | | [79-97] ORF |
| 544 | GACCUGGGAGAUAAUUCUA | UAGAAUUAUCUCCCAGGUC | | [4200-4218] 3′UTR |
| 545 | GAAAUUUGAAUACUUGCAG | CUGCAAGUAUUCAAAUUUC | | [4153-4171] 3′UTR |
| 546 | GGAAUGGUGUGUGAAUGCC | GGCAUUCACACACCAUUCC | | [383-401] ORF |
| 547 | GUGGAAUGGUGUGUGAAUG | CAUUCACACACCAUUCCAC | | [381-399] ORF |
| 548 | GUUUCUCCAAGGUUUUCUA | UAGAAAACCUUGGAGAAAC | | [3838-3856] 3′UTR |
| 549 | GUGCGAAUAUAGCCUUUCU | AGAAAGGCUAUAUUCGCAC | | [3565-3583] 3′UTR |
| 550 | UAGGAGGAAAAGGAAAUAA | UUAUUUCCUUUUCCUCCUA | | [3209-3227] 3′UTR |
| 551 | UUAGGAGGAAAAGGAAAUA | UAUUUCCUUUUCCUCCUAA | | [3208-3226] 3′UTR |
| 552 | GGUUUAGGAGGAAAAGGAA | UUCCUUUUCCUCCUAAACC | | [3205-3223] 3′UTR |
| 553 | UGGUUUAGGAGGAAAAGGA | UCCUUUUCCUCCUAAACCA | | [3204-3222] 3′UTR |
| 554 | CCUUAUUUUCCGUAAAUAC | GUAUUUACGGAAAAUAAGG | | [2851-2869] 3′UTR |
| 555 | GGUUCUGCACAUUCUUAUU | AAUAAGAAUGUGCAGAACC | | [2623-2641] 3′UTR |
| 556 | GGUAACUGGGCCAGAUGUU | AACAUCUGGCCCAGUUACC | | [2336-2354] 3′UTR |
| 557 | GCAUACUCCAGUUUACUAA | UUAGUAAACUGGAGUAUGC | | [2309-2327] 3′UTR |
| 558 | GUCAUUUUUGCCAUCAUUA | UAAUGAUGGCAAAAAUGAC | | [2199-2217] 3′UTR |
| 559 | GAAGAAGGAAACCAAGGAG | CUCCUUGGUUUCCUUCUUC | | [2158-2176] 3′UTR |
| 560 | AGGAAAACUUCUAACUUGU | ACAAGUUAGAAGUUUUCCU | | [1714-1732] ORF + 3′UTR |
| 561 | ACAAAAGACUUUGUAUGGA | UCCAUACAAAGUCUUUUGU | | [1532-1550] ORF |
| 562 | AGCUACAACAUCUACCUCA | UGAGGUAGAUGUUGUAGCU | | [1437-1455] ORF |
| 563 | GGUGAUGUUAGUGGGAGCA | UGCUCCCACUAACAUCACC | Rabbit | [1223-1241] ORF |
| 564 | CUAUGAGGUGGUGAUGUUA | UAACAUCACCACCUCAUAG | Rat, Rabbit, Ms | [1214-1232] ORF |
| 565 | UGCCAGUGAAGAUGUGUUC | GAACACAUCUUCACUGGCA | | [1193-1211] ORF |
| 566 | UUAGUAUCCAUAUCCGCAU | AUGCGGAUAUGGAUACUAA | | [1066-1084] ORF |
| 567 | CUUCUUUAGUAUCCAUAUC | GAUAUGGAUACUAAAGAAG | | [1061-1079] ORF |
| 568 | CCCUUUUACACUGACAUCC | GGAUGUCAGUGUAAAAGGG | | [1028-1046] ORF |
| 569 | GUGGCACCCUUUUACACUG | CAGUGUAAAAGGGUGCCAC | | [1022-1040] ORF |
| 570 | GACUUGGAAAUGGAUAGUG | CACUAUCCAUUUCCAAGUC | Ms | [818-836] ORF |
| 571 | GUAUUACCGGGUUUAUGAU | AUCAUAAACCCGGUAAUAC | | [98-116] ORF |
| 572 | GGCAUCACUGGAGUUGUCA | UGACAACUCCAGUGAUGCC | | [540-558] ORF |
| 573 | CCUCUUUGUCUGGUAUUAC | GUAAUACCAGACAAAGAGG | | [86-104] ORF |
| 574 | GUCUUCCUCUUUGUCUGGU | ACCAGACAAAGAGGAAGAC | | [81-99] ORF |
| 575 | ACCUGGGAGAUAAUUCUAC | GUAGAAUUAUCUCCCAGGU | | [4201-4219] 3′UTR |
| 576 | GGGACCUGGGAGAUAAUUC | GAAUUAUCUCCCAGGUCCC | | [4198-4216] 3′UTR |
| 577 | UUGAAUACUUGCAGGCUUU | AAAGCCUGCAAGUAUUCAA | | [4158-4176] 3′UTR |
| 578 | ACAGGUACUCUAAGAAUGA | UCAUUCUUAGAGUACCUGU | | [4063-4081] 3′UTR |
| 579 | UCCUUUUUAUCUCUCAAGC | GCUUGAGAGAUAAAAAGGA | | [3958-3976] 3′UTR |
| 580 | UUCCUUUUUAUCUCUCAAG | CUUGAGAGAUAAAAAGGAA | | [3957-3975] 3′UTR |
| 581 | GUUUCCUUUUUAUCUCUCA | UGAGAGAUAAAAAGGAAAC | | [3955-3973] 3′UTR |
| 582 | CUGUGCGAAUAUAGCCUUU | AAAGGCUAUAUUCGCACAG | | [3563-3581] 3′UTR |
| 583 | GGAGGAAAAGGAAAUAAGG | CCUUAUUUCCUUUUCCUCC | | [3211-3229] 3′UTR |
| 584 | GGGAACUAAAACCAGUUUU | AAAACUGGUUUUAGUUCCC | | [3092-3110] 3′UTR |
| 585 | AGGGUUGGAGGUGGAGAAU | AUUCUCCACCUCCAACCCU | | [2858-2676] 3′UTR |
| 586 | AGGGUUCUGCACAUUCUUA | UAAGAAUGUGCAGAACCCU | | [2621-2639] 3′UTR |
| 587 | GGAUUGCCUGAAGGGUUCU | AGAACCCUUCAGGCAAUCC | | [2810-2628] 3′UTR |
| 588 | UCAAGAACAGGCUAAGGAU | AUCCUUAGCCUGUUCUUGA | | [2580-2598] 3′UTR |
| 589 | GUCAAGAACAGGCUAAGGA | UCCUUAGCCUGUUCUUGAC | | [2579-2997] 3′UTR |
| 590 | CUAGGGUCAAGAACAGGCU | AGCCUGUUCUUGACCCUAG | | [2574-2592] 3′UTR |
| 591 | CUGGGCCAGAUGUUCUUUC | GAAAGAACAUCUGGCCCAG | | [2341-2359] 3′UTR |
| 592 | CUCCAGUUUACUAAUACAG | CUGUAUUAGUAAACUGGAG | | [2314-2332] 3′UTR |
| 593 | GGACAUAAUACAUGUUGAG | CUCAACAUGUAUUAUGUCC | | [2251-2269] 3′UTR |
| 594 | GGAAAUUUUCCAGAUCAUU | AAUGAUCUGGAAAAUUUCC | | [2231-2249] 3′UTR |
| 595 | CUACUUUGUGUCAUUUUUG | CAAAAAUGACACAAAGUAG | | [2190-2208] 3′UTR |
| 596 | AGUAAUGUCAGUGGAAGUU | AACUUCCACUGACAUUACU | | [1958-1976] 3′UTR |
| 597 | CUUAAAGGAAUGUCAAAGA | UCUUUGACAUUCCUUUAAG | | [1853-1871] 3′UTR |
| 598 | CCUGAAGCCUUGGCUGAAA | UUUCAGCCAAGGCUUCAGG | Rabbit, Ms | [1629-1647] ORF |
| 599 | UGCAAGUCAACACCCUAAU | AUUAGGGUGUUGACUUGCA | Rabbit | [1583-1601] ORF |
| 600 | CAAAAGACUUUGUAUGGAC | GUCCAUACAAAGUCUUUUG | | [1533-1551] ORF |
| 601 | CUACAACAUCUACCUCACU | AGUGAGGUAGAUGUUGUAG | | [1439-1457] ORF |
| 602 | GGGAAAAAUAAAGGAAUGC | GCAUUCCUUUAUUUUUCCC | | [767-785] ORF |
| 603 | UGGAGCUGAACGAAUUGUA | UACAAUUCGUUCAGCUCCA | Rat, Rabbit, Ms | [680-698] ORF |
| 604 | UCUCCAAGGUUUUCUAUCU | AGAUAGAAAACCUUGGAGA | | [3841-3859] 3′UTR |
| 605 | GCUGUGCGAAUAUAGCCUU | AAGGCUAUAUUCGCACAGC | | [3562-3580] 3′UTR |
| 606 | GAAGACUGGUUUAGGAGGA | UCCUCCUAAACCAGUCUUC | | [3198-3216] 3′UTR |
| 607 | UUCUCCAGCUAGCCCUUAU | AUAAGGGCUAGCUGGAGAA | | [2763-2781] 3′UTR |
| 608 | CCUGCUCCUUGUGCUAUAA | UUAUAGCACAAGGAGCAGG | | [2709-2727] 3′UTR |
| 609 | CCAAUAGGAUUGCCUGAAG | CUUCAGGCAAUCCUAUUGG | | [2604-2622] 3′UTR |
| 610 | AGAACAGGCUAAGGAUACU | AGUAUCCUUAGCCUGUUCU | | [2583-2601] 3′UTR |
| 611 | ACUCCAGUUUACUAAUACA | UGUAUUAGUAAACUGGAGU | | [2313-2331] 3′UTR |
| 612 | CAGAUCAUUAGGACAUAAU | AUUAUGUCCUAAUGAUCUG | | [2241-2259] 3′UTR |
| 613 | AAGGAAAUUUUCCAGAUCA | UGAUCUGGAAAAUUUCCUU | | [2229-2247] 3′UTR |
| 614 | GAAUGUCAAAGAUUGUUUG | CAAACAAUCUUUGACAUUC | | [1860-1878] 3′UTR |
| 615 | ACUUCUAACUUGUCUCUUC | GAAGAGACAAGUUAGAAGU | | [1720-1738] ORF + 3′UTR |
| 616 | AAGGAAAACUUCUAACUUG | CAAGUUAGAAGUUUUCCUU | | [1713-1731] ORF + 3′UTR |
| 617 | CCAAUCACUUUGCUGUGCA | UGCACAGCAAAGUGAUUGG | Rat, Rabbit, Ms | [1477-1495] ORF |
| 618 | GUGGUGAUGUUAGUGGGAG | CUCCCACUAACAUCACCAC | Rat, Rabbit, Ms | [1221-1239] ORF |
| 619 | GUGGGUCCCAUGUUUCUGU | ACAGAAACAUGGGACCCAC | | [834-852] ORF |
| 620 | UGUCUGGUAUUACCGGGUU | AACCCGGUAAUACCAGACA | | [92-110] ORF |
| 621 | CCCUGUGUUAUCCAGUUAU | AUAACUGGAUAACACAGGG | | [3898-3914] 3′UTR |
| 622 | GCCCUGUGUUAUCCAGUUA | UAACUGGAUAACACAGGGC | | [3895-3913] 3′UTR |
| 623 | AAGACUGGUUUAGGAGGAA | UUCCUCCUAAACCAGUCUU | | [3199-3217] 3′UTR |
| 624 | GGAAGAAGGAGAAAGAGCU | AGCUCUUUCUCCUUCUUCC | | [3167-3185] 3′UTR |
| 625 | AUCAUCAUCAUCAUCCUAA | UUAGGAUGAUGAUGAUGAU | | [2819-2937] 3′UTR |
| 626 | AACAGGCUAAGGAUACUAA | UUAGUAUCCUUAGCCUGUU | | [2585-2803] 3′UTR |
| 627 | CCAGUUUACUAAUACAGCA | UGCUGUAUUAGUAAACUGG | | [2316-2334] 3′UTR |
| 628 | UCCAGAUCAUUAGGACAUA | UAUGUCCUAAUGAUCUGGA | | [2239-2257] 3′UTR |
| 629 | UGUGUCAUUUUUGCCAUCA | UGAUGGCAAAAAUGACACA | | [2196-2214] 3′UTR |
| 630 | UCAUUUUUUUCCUCUCAGU | ACUGAGAGGAAAAAAAUGA | | [1942-1960] 3′UTR |
| 631 | CCCUGAGUAAACAAAGCAU | AUGCUUUGUUUACUCAGGG | | [1648-1666] ORF |
| 632 | GGGACACACAUGCCUUUGA | UCAAAGGCAUGUGUGUCCC | | [1351-1369] ORF |
| 633 | UCAAGUCAGUCUGGUACAA | UUGUACCAGACUGACUUGA | Rabbit | [1273-1291] ORF |
| 634 | CGUGGAAACUACCUAAGAU | AUCUUAGGUAGUUUCCACG | | [1150-1168] ORF |
| 635 | CAAUGCUUGUGGCUGUGAU | AUCACAGCCACAAGCAUUG | Rabbit, Ms | [1112-1130] ORF |
| 636 | CCAUCGAGCUACAGAUGAA | UUCAUCUGUAGCUCGAUGG | | [934-952] ORF |
| 637 | GGAGCUGAACGAAUUGUAC | GUACAAUUCGUUCAGCUCC | Rat, Rabbit, Ms | [681-699] ORF |
| 638 | UUCUUCUGCUUUUGCAAUU | AAUUGCAAAAGCAGAAGAA | | [4126-4144] 3′UTR |
| 639 | UCGCAUCUGUGUGACUAAU | AUUAGUCACACAGAUGCGA | | [2884-2902] 3′UTR |
| 640 | UCCUGCCGAAAGUGGUCUU | AAGACCACUUUGGGCAGGA | | [2746-2764] 3′UTR |
| 641 | AGACUAAUUUCCUGCCCAA | UUGGGCAGGAAAUUAGUCU | | [2737-2755] 3′UTR |
| 642 | CUGCUCCUUGUGCUAUAAA | UUUAUAGCACAAGGAGCAG | | [2710-2728] 3′UTR |
| 643 | UCCUGCUCCUUGUGCUAUA | UAUAGCACAAGGAGCAGGA | | [2708-2726] 3′UTR |
| 644 | ACACCCUAAUACCAGAAUA | UAUUCUGGUAUUAGGGUGU | Rabbit | [1592-1610] ORF |
| 645 | GUACCUCAGUUAUAAGGAG | CUCCUUAUAACUGAGGUAC | | [3750-3768] 3′UTR |
| 646 | GGAGAGGGUACCUCAGUUA | UAACUGAGGUACCCUCUCC | | [3743-3761] 3′UTR |
| 647 | UGAACGUCUUCCUCUUUGU | ACAAAGAGGAAGACGUUCA | Rabbit | [76-94] ORF |
| 648 | GAGGUAUACACAUACUGAU | AUCAGUAUGUGUAUACCUC | | [3359-3377] 3′UTR |
| 649 | CAAGAGAGAGUCUCUAAAU | AUUUAGAGACUCUCUCUUG | | [3013-3031] 3′UTR |
| 650 | UCAAGAGAGAGUCUCUAAA | UUUAGAGACUCUCUCUUGA | | [3012-3030] 3′UTR |
| 651 | CUGAGCAUACUCCAGUUUA | UAAACUGGAGUAUGCUCAG | | [2305-2323] 3′UTR |
| 652 | GGCUGAAACCCUGAGUAAA | UUUACUCAGGGUUUCAGCC | | [1640-1858] ORF |
| 653 | UUGGCUGAAACCCUGAGUA | UACUCAGGGUUUCAGCCAA | | [1638-1656] ORF |
| 654 | GGCCCAACUGGGAUAAUGA | UCAUUAUCCCAGUUGGGCC | | [1552-1570] ORF |
| 655 | UCACACCCUUCGCAUCCAU | AUGGAUGCGAAGGGUGUGA | | [1252-1270] ORF |
| 656 | GUGGAAACUACCUAAGAUA | UAUCUUAGGUAGUUUCCAC | | [1151-1169] ORF |
| 657 | GGACAAUACAUUUUUGUCA | UGACAAAAAUGUAUUGUCC | | [978-996] ORF |
| 658 | AUCAUCUCUUUGUGAUCUU | AAGAUCACAAAGAGAUGAU | | [640-658] ORF |
| 659 | CUGAGAAUAUUGGCCCUUU | AAAGGGCCAAUAUUCUCAG | | [3770-3788] 3′UTR |
| 660 | CGCAUCUGUGUGACUAAUG | CAUUAGUCACACAGAUGCG | | [2885-2903] 3′UTR |
| 661 | CGAAGACAACUGGACAGGA | UCCUGUCCAGUUGUCUUCG | Rat, Ms | [285-303] ORF |
| 662 | AGGACAUAAUACAUGUUGA | UCAACAUGUAUUAUGUCCU | | [2250-2268] 3′UTR |
| 663 | UGAUACUCUUUCCACAAUA | UAUUGUGGAAAGAGUAUCA | | [2092-2110] 3′UTR |
| 664 | CUGAAGCCUUGGCUGAAAC | GUUUCAGCCAAGGCUUCAG | Rabbit, Ms | [1630-1648] ORF |
| 665 | GCCCAACUGGGAUAAUGAA | UUCAUUAUCCCAGUUGGGC | | [1553-1571] ORF |
| 666 | CCUGCCUGAAUUUCAACUG | CAGUUGAAAUUCAGGCAGG | Rat, Ms | [187-205] ORF |
| 667 | CUGGUACAAAUAUUGCAAU | AUUGCAAUAUUUGUACCAG | | [1283-1301] ORF |
| 668 | GCGUGGAAACUACCUAAGA | UCUUAGGUAGUUUCCACGC | | [1149-1167] ORF |
| 669 | AGAAGGUGGUCAUCACCAA | UUGGUGAUGACCACCUUCU | Rabbit | [892-910] ORF |
| 670 | CGAAUUGUACGUGGGCAGA | UCUGCCCACGUACAAUUCG | | [890-708] ORF |
| 671 | ACGAAUUGUACGUGGGCAG | CUGCCCACGUACAAUUCGU | | [689-707] ORF |
| 672 | AGCUGAACGAAUUGUACGU | ACGUACAAUUCGUUCAGCU | Rat, Rabbit, Ms | [683-701] ORF |
| 673 | CGAGUCAAUAAUUCUGAUC | GAUCAGAAUUAUUGACUCG | | [402-420] ORF |
| 674 | CUUCUGCUUUUGCAAUUGU | ACAAUUGCAAAAGCAGAAG | | [4128-4146] 3′UTR |
| 675 | CUAUGAGUCAGGCUCUGUU | AACAGAGCCUGACUCAUAG | | [3690-3708] 3′UTR |
| 676 | UGCGAAUAUAGCCUUUCUG | CAGAAAGGCUAUAUUCGCA | | [3566-3584] 3′UTR |
| 677 | GGAGGUAUACACAUACUGA | UCAGUAUGUGUAUACCUCC | | [3358-3376] 3′UTR |
| 678 | CCAUUUCUGGGAACUAAAA | UUUUAGUUCCCAGAAAUGG | | [3084-3102] 3′UTR |
| 679 | UGCCCAAAGUGGUCUUCUC | GAGAAGACCACUUUGGGCA | | [2749-2767] 3′UTR |
| 680 | UGCUCCUUGUGCUAUAAAA | UUUUAUAGCACAAGGAGCA | | [2711-2729] 3′UTR |
| 681 | CCAUGACUCUGGCUCAUCU | AGAUGAGCCAGAGUCAUGG | | [2687-2705] 3′UTR |
| 682 | ACCAAUAGGAUUGCCUGAA | UUCAGGCAAUCCUAUUGGU | | [2603-2621] 3′UTR |
| 683 | GCCAGAUGUUCUUUCUACA | UGUAGAAAGAACAUCUGGC | | [2345-2363] 3′UTR |
| 684 | AUCUGAGCAUACUCCAGUU | AACUGGAGUAUGCUCAGAU | | [2303-2321] 3′UTR |
| 685 | GAAAUCUGCUGUCCUUCCU | AGGAAGGACAGCAGAUUUC | | [232-250] ORF |
| 686 | UGUAAUGGUUUUCCCUUAA | UUAAGGGAAAACCAUUACA | | [1839-1857] 3′UTR |
| 687 | UGGCUGAAACCCUGAGUAA | UUACUCAGGGUUUCAGCCA | | [1639-1657] ORF |
| 688 | CAAGUCAACACCCUAAUAC | GUAUUAGGGUGUUGACUUG | Rabbit | [1585-1603] ORF |
| 689 | CCCAACUGGGAUAAUGAAU | AUUCAUUAUCCCAGUUGGG | | [1554-1572] ORF |
| 690 | ACGGCCCAACUGGGAUAAU | AUUAUCCCAGUUGGGCCGU | | [1550-1568] ORF |
| 691 | CAAUCACUUUGCUGUGCAC | GUGCACAGCAAAGUGAUUG | Rat, Rabbit, Ms | [1478-1498] ORF |
| 692 | CUGCAGCCUGCCUGAAUUU | AAAUUCAGGCAGGCUGCAG | Rat, Ms | [181-199] ORF |
| 693 | UGCGUGGAAACUACCUAAG | CUUAGGUAGUUUCCACGCA | | [1146-1166] ORF |
| 694 | UCAAAACCAUCGAGCUACA | UGUAGCUCGAUGGUUUUGA | | [928-948] ORF |
| 695 | UCAGUUUGCUGGAAACCCU | AGGGUUUCCAGCAAACUGA | | [794-812] ORF |
| 696 | UUGCUCGAAAGAGAAUAAA | UUUAUUCUCUUUCGAGCAA | | [478-496] ORF |
| 697 | UGCCCUGUGUUAUCCAGUU | AACUGGAUAACACAGGGCA | | [3894-3912] 3′UTR |
| 698 | UGCAUAAUUAAAACCAGCU | AGCUGGUUUUAAUUAUGCA | | [3799-3817] 3′UTR |
| 699 | GGCCCUUUCUAACCUAUGU | ACAUAGGUUAGAAAGGGCC | | [3781-3799] 3′UTR |
| 700 | CAAUUCUUUACACCAAAGU | ACUUUGGUGUAAAGAAUUG | | [3717-3735] 3′UTR |
| 701 | UCACAAAUUUUGAUGCUGA | UCAGCAUCAAAAUUUGUGA | | [3420-3438] 3′UTR |
| 702 | UCAUAUUGGUCAUUCCCUG | CAGGGAAUGACCAAUAUGA | | [3296-3314] 3′UTR |
| 703 | CCCAUUUCUGGGAACUAAA | UUUAGUUCCCAGAAAUGGG | | [3083-3101] 3′UTR |
| 704 | UCUUAGAACUGCUCCCAUU | AAUGGGAGCAGUUCUAAGA | | [3070-3088] 3′UTR |
| 705 | CCAGAAACAAAAAUCUCUA | UAGAGAUUUUUGUUUCUGG | | [2948-2966] 3′UTR |
| 706 | GUGACAAAUAUGUAUCUGA | UCAGAUACAUAUUUGUCAC | | [2802-2820] 3′UTR |
| 707 | UCACCUUUCAUAAAAUGGU | ACCAUUUUAUGAAAGGUGA | | [307-325] ORF |
| 708 | AGGAAUCUCACCUUUCAUA | UAUGAAAGGUGAGAUUCCU | | [300-318] ORF |
| 709 | GGAUACUAACCAAUAGGAU | AUCCUAUUGGUUAGUAUCC | | [2595-2613] 3′UTR |
| 710 | GGAUACUCAAGUAAGUUUU | AAAACUUACUUGAGUAUCC | | [2430-2448] 3′UTR |
| 711 | UGAGCAUACUCCAGUUUAC | GUAAACUGGAGUAUGCUCA | | [2306-2324] 3′UTR |
| 712 | GAGAGUGUCUCAACACUUA | UAAGUGUUGAGACACUCUC | | [2267-2285] 3′UTR |
| 713 | UUCCAGAUCAUUAGGACAU | AUGUCCUAAUGAUCUGGAA | | [2238-2256] 3′UTR |
| 714 | UCAGUAACUUCCCUAAGAU | AUCUUAGGGAAGUUACUGA | | [2032-2050] 3′UTR |
| 715 | UUCAUUUUUUUCCUCUCAG | CUGAGAGGAAAAAAAUGAA | | [1941-1959] 3′UTR |
| 716 | GCAUCUCCAACUCUGAGUC | GACUCAGAGUUGGAGAUGC | Rat, Rabbit | [1683-1681] ORF |
| 717 | AUACCAGAAUAGGAGUUUU | AAAACUCCUAUUCUGGUAU | Rabbit | [1600-1618] ORF |
| 718 | UAAUACCAGAAUAGGAGUU | AACUCCUAUUCUGGUAUUA | Rabbit | [1598-1616] ORF |
| 719 | UGCACCAUGAUGAGGAGAA | UUCUCCUCAUCAUGGUGCA | Rabbit, Ms | [1492-1510] ORF |
| 720 | AGGCCAAUCACUUUGCUGU | ACAGCAAAGUGAUUGGCCU | Rat, Rabbit, Ms | [1474-1492] ORF |
| 721 | ACCUAAGAUAGCGGUUGAU | AUCAACCGCUAUCUUAGGU | | [1160-1178] ORF |
| 722 | CUACCUAAGAUAGCGGUUG | CAACCGCUAUCUUAGGUAG | | [1158-1176] ORF |
| 723 | GUGAUAAGCAGGAGUUUCA | UGAAACUCCUGCUUAUCAC | Rat, Rabbit | [1126-1144] ORF |
| 724 | CUGUGAUAAGCAGGAGUUU | AAACUCCUGCUUAUCACAG | Rat, Rabbit | [1124-1142] ORF |
| 725 | CUGGAGUGGCACCCUUUUA | UAAAAGGGUGCCACUCCAG | | [1017-1035] ORF |
| 726 | UCCCUCAGUUUGCUGGAAA | UUUCCAGCAAACUGAGGGA | | [790-808] ORF |
| 727 | ACCAAAACCAUCCGGAGGU | ACCUCCGGAUGGUUUUGGU | | [594-612] ORF |
| 728 | GCAAAAUGAAAGUUAUCUC | GAGAUAACUUUCAUUUUGC | | [455-473] ORF |
| 729 | UCAGUUAUAAGGAGUCUGA | UCAGACUCCUUAUAACUGA | | [3755-3773] 3′UTR |
| 730 | CCUCAGUUAUAAGGAGUCU | AGACUCCUUAUAACUGAGG | | [3753-3771] 3′UTR |
| 731 | CUUUACACCAAAGUAUGAA | UUCAUACUUUGGUGUAAAG | | [3722-3740] 3′UTR |
| 732 | CUAAAUAAGAACUCAGUUG | CAACUGAGUUCUUAUUUAG | | [3467-3485] 3′UTR |
| 733 | ACACAUACUGAUGUUGUUU | AAACAACAUCAGUAUGUGU | | [3366-3384] 3′UTR |
| 734 | GUAUACACAUACUGAUGUU | AACAUCAGUAUGUGUAUAC | | [3362-3380] 3′UTR |
| 735 | UCCCAUUUCUGGGAACUAA | UUAGUUCCCAGAAAUGGGA | | [3082-3100] 3′UTR |
| 736 | GCAGACUAAUUUCCUGCCC | GGGCAGGAAAUUAGUCUGC | | [2735-2753] 3′UTR |
| 737 | UGCCAUGACUCUGGCUCAU | AUGAGCCAGAGUCAUGGCA | | [2685-2703] 3′UTR |
| 738 | GAAUUAACCUCCUGCCAUG | CAUGGCAGGAGGUUAAUUC | | [2673-2691] 3′UTR |
| 739 | UGGACAGGAAUCUCACCUU | AAGGUGAGAUUCCUGUCCA | | [295-313] ORF |
| 740 | CUGUCAUAGACCAAAGGAA | UUCCUUUGGUCUAUGACAG | | [2544-2562] 3′UTR |
| 741 | CGGAUACUCAAGUAAGUUU | AAACUUACUUGAGUAUCCG | | [2429-2447] 3′UTR |
| 742 | GGAGUUAAUGUAAUACUCA | UGAGUAUUACAUUAACUCC | | [2383-2401] 3′UTR |
| 743 | AGCAUACUCCAGUUUACUA | UAGUAAACUGGAGUAUGCU | | [2308-2326] 3′UTR |
| 744 | CUGAAGGAAAUUUUCCAGA | UCUGGAAAAUUUCCUUCAG | | [2226-2244] 3′UTR |
| 745 | CCCAAAAGAAGAAGGAAAC | GUUUCCUUCUUCUUUUGGG | | [2151-2169] 3′UTR |
| 746 | AUACUGAGCUGCCUCAGAA | UUCUGAGGCAGCUCAGUAU | | [2106-2126] 3′UTR |
| 747 | GCCCAUCUUACUCCAGGUU | AACCUGGAGUAAGAUGGGC | | [2073-2091] 3′UTR |
| 748 | GGACUCAAUUUUAGAAUCA | UGAUUCUAAAAUUGAGUCC | | [1992-2010] 3′UTR |
| 749 | AUGUAAUGGUUUUCCCUUA | UAAGGGAAAACCAUUACAU | | [1838-1856] 3′UTR |
| 750 | CACCCUAAUACCAGAAUAG | CUAUUCUGGUAUUAGGGUG | Rabbit | [1593-1611] ORF |
| 751 | AACACCCUAAUACCAGAAU | AUUCUGGUAUUAGGGUGUU | Rabbit | [1591-1609] ORF |
| 752 | UCACUUUGCUGUGCACCAU | AUGGUGCACAGCAAAGUGA | Rabbit, Ms | [1481-1499] ORF |
| 753 | AGAGCCAGAUGCAGGAAAG | CUUUCCUGCAUCUGGCUCU | | [1399-1417] ORF |
| 754 | GAGAGCCAGAUGCAGGAAA | UUUCCUGCAUCUGGCUCUC | | [1398-1416] ORF |
| 755 | UUGCUGGAAACCCUCCUAU | AUAGGAGGGUUUCCAGCAA | | [799-817] ORF |
| 756 | CCCUCAGUUUGCUGGAAAC | GUUUCCAGCAAACUGAGGG | | [791-809] ORF |
| 757 | AAAUAAAGGAAUGCCCAAU | AUUGGGCAUUCCUUUAUUU | | [772-790] ORF |
| 758 | GUCUGGUAUUACCGGGUUU | AAACCCGGUAAUACCAGAC | | [93-111] ORF |
| 759 | ACCAUCCGGAGGUCUUACU | AGUAAGACCUCCGGAUGGU | | [600-618] ORF |
| 760 | UGCUUUUGCAAUUGUGUUU | AAACACAAUUGCAAAAGCA | | [4132-4150] 3′UTR |
| 761 | CCGAGAGUGUUUCUCCAAG | CUUGGAGAAACACUCUCGG | | [3830-3848] 3′UTR |
| 762 | UUGGCCCUUUCUAACCUAU | AUAGGUUAGAAAGGGCCAA | | [3779-3797] 3′UTR |
| 763 | UCUGAGAAUAUUGGCCCUU | AAGGGCCAAUAUUCUCAGA | | [3769-3787] 3′UTR |
| 764 | ACCUCAGUUAUAAGGAGUC | GACUCCUUAUAACUGAGGU | | [3752-3770] 3′UTR |
| 765 | GCACAUCUAUUUAAUGUGG | CCACAUUAAAUAGAUGUGC | | [366-384] ORF |
| 766 | CUGACAAAUAAGGCCUGCU | AGCAGGCCUUAUUUGUCAG | | [3546-3564] 3′UTR |
| 767 | GAACUAAAUAAGAACUCAG | CUGAGUUCUUAUUUAGUUC | | [3464-3482] 3′UTR |
| 768 | CACAUACUGAUGUUGUUUU | AAAACAACAUCAGUAUGUG | | [3387-3385] 3′UTR |
| 769 | AGGAGGUAUACACAUACUG | CAGUAUGUGUAUACCUCCU | | [3357-3375] 3′UTR |
| 770 | GCCAAGAGCAGGGUUUUCU | AGAAAACCCUGCUCUUGGC | | [3045-3063] 3′UTR |
| 771 | CACCUUUCAUAAAAUGGUG | CACCAUUUUAUGAAAGGUG | | [308-326] ORF |
| 772 | UCUCACCUUUCAUAAAAUG | CAUUUUAUGAAAGGUGAGA | | [305-323] ORF |
| 773 | CAGACUAAUUUCCUGCCCA | UGGGCAGGAAAUUAGUCUG | | [2736-2754] 3′UTR |
| 774 | GUGGAGAAUUAACCUCCUG | CAGGAGGUUAAUUCUCCAC | | [2668-2686] 3′UTR |
| 775 | GCUAAGGAUACUAACCAAU | AUUGGUUAGUAUCCUUAGC | | [2590-2608] 3′UTR |
| 776 | UGGAGUUAAUGUAAUACUC | GAGUAUUACAUUAACUCCA | | [2382-2400] 3′UTR |
| 777 | CAGUUUACUAAUACAGCAG | CUGCUGUAUUAGUAAACUG | | [2317-2335] 3′UTR |
| 778 | UCUGAGCAUACUCCAGUUU | AAACUGGAGUAUGCUCAGA | | [2304-2322] 3′UTR |
| 779 | ACCCUGAGUAAACAAAGCA | UGCUUUGUUUACUCAGGGU | | [1647-1685] ORF |
| 780 | GCCUGAAACAAAAGACUUU | AAAGUCUUUUGUUUCAGGC | | [1525-1543] ORF |
| 781 | GCCUGCCUGAAUUUCAACU | AGUUGAAAUUCAGGCAGGC | Rat, Ms | [186-204] ORF |
| 782 | ACCAAUCUGAAGCUCAAAA | UUUUGAGCUUCAGAUUGGU | | [1306-1326] ORF |
| 783 | GGGCUGUUCAAUGCUUGUG | CACAAGCAUUGAACAGCCC | Rabbit | [1104-1122] ORF |
| 784 | CCCUUUCAAAACCAUCGAG | CUCGAUGGUUUUGAAAGGG | | [923-941] ORF |
| 785 | GGAUAGUGGGUCCCAUGUU | AACAUGGGACCCACUAUCC | Rabbit | [829-847] ORF |
| 786 | GCUGUGCAUAAUAUAACAG | CUGUUAUAUUAUGCACAGC | | [723-741] ORF |
| 787 | UCACGCUGUGCCUCAUAUU | AAUAUGAGGCACAGCGUGA | | [559-577] ORF |
| 788 | GAGGGUACCUCAGUUAUAA | UUAUAACUGAGGUACCCUC | | [3746-3764] 3′UTR |
| 789 | ACACUUAGGUCCAGCCUGU | ACAGGCUGGACCUAAGUGU | | [3614-3632] 3′UTR |
| 790 | UGACAAAUAAGGCCUGCUG | CAGCAGGCCUUAUUUGUCA | | [3547-3565] 3′UTR |
| 791 | GUCUGACAAAUAAGGCGUG | CAGGCCUUAUUUGUCAGAC | | [3544-3562] 3′UTR |
| 792 | GUCACAAAUUUUGAUGCUG | CAGCAUCAAAAUUUGUGAC | | [3419-3437] 3′UTR |
| 793 | GGAUGAUUGCACUUCACUC | GAGUGAAGUGCAAUCAUCC | | [331-349] ORF |
| 794 | GCAUGGAUGAUUGCACUUC | GAAGUGCAAUCAUCCAUGC | | [327-345] ORF |
| 795 | UGGGAACUAAAACCAGUUU | AAACUGGUUUUAGUUCCCA | | [3091-3109] 3′UTR |
| 796 | UGGCCAAGAGCAGGGUUUU | AAAACCCUGCUCUUGGCCA | | [3043-3061] 3′UTR |
| 797 | AGAUCAAAUUCACACUCAA | UUGAGUGUGAAUUUGAUCU | | [2972-2990] 3′UTR |
| 798 | GCAUCUGUGUGACUAAUGG | CCAUUAGUCACACAGAUGC | | [2888-2904] 3′UTR |
| 799 | CGUAAAUACACACACAAAA | UUUUGUGUGUGUAUUUACG | | [2881-2879] 3′UTR |
| 800 | CCGUAAAUACACACACAAA | UUUGUGUGUGUAUUUACGG | | [2860-2878] 3′UTR |
| 801 | CCAAAGUGGUCUUCUCCAG | CUGGAGAAGACCACUUUGG | | [2752-2770] 3′UTR |
| 802 | CAGGAAUCUCACCUUUCAU | AUGAAAGGUGAGAUUCCUG | | [299-317] ORF |
| 803 | AGGAUUGCCUGAAGGGUUC | GAACCCUUCAGGCAAUCCU | | [2609-2627] 3′UTR |
| 804 | GGCUAAGGAUACUAACCAA | UUGGUUAGUAUCCUUAGCC | | [2589-2607] 3′UTR |
| 805 | CUAUUACUGUCAUAGACCA | UGGUCUAUGACAGUAAUAG | | [2538-2558] 3′UTR |
| 806 | UGUUCUUUCUACAGAAGAA | UUCUUCUGUAGAAAGAACA | | [2351-2389] 3′UTR |
| 807 | AGAUGUUCUUUCUACAGAA | UUCUGUAGAAAGAAGAUCU | | [2348-2368] 3′UTR |
| 808 | AGAGUGUCUCAACACUUAU | AUAAGUGUUGAGACACUCU | | [2268-2288] 3′UTR |
| 809 | UGCUCAACAAGAGUUCGAA | UUCGAACUCUUGUUGAGCA | | [270-288] ORF |
| 810 | GGUUCAGUAACUUCCCUAA | UUAGGGAAGUUACUGAACC | | [2029-2047] 3′UTR |
| 811 | CUGCCAAAUGCUCAAAUAA | UUAUUUGAGCAUUUGGCAG | | [1764-1782] 3′UTR |
| 812 | GUGCUGCCAAAUGCUCAAA | UUUGAGCAUUUGGCAGCAC | | [1761-1779] 3′UTR |
| 813 | UCUCUUCCAUGAGGAAAUA | UAUUUCCUCAUGGAAGAGA | | [1732-1750] 3′UTR |
| 814 | UGAGUAAACAAAGCAUCUC | GAGAUGCUUUGUUUACUCA | | [1651-1669] ORF |
| 815 | GAAUAGGAGUUUUCCUCUG | CAGAGGAAAACUCCUAUUC | Rabbit | [1606-1624] ORF |
| 816 | CCCUAAUACCAGAAUAGGA | UCCUAUUCUGGUAUUAGGG | Rabbit | [1595-1613] ORF |
| 817 | UCAACUGCAUGCUGAUUCU | AGAAUCAGCAUGCAGUUGA | | [199-217] ORF |
| 818 | CCAUUUUUGUCAUUCUGGU | ACCAGAAUGACAAAAAUGG | | [46-64] ORF |
| 819 | GGCCAAUCACUUUGCUGUG | CACAGCAAAGUGAUUGGCC | Rat, Rabbit, Ms | [1475-1493] ORF |
| 820 | UGCCUGAAUUUCAACUGCA | UGCAGUUGAAAUUCAGGCA | Rat | [189-207] ORF |
| 821 | CAGCCUGCCUGAAUUUCAA | UUGAAAUUCAGGCAGGCUG | Rat, Ms | [184-202] ORF |
| 822 | GCUGUGAUAAGCAGGAGUU | AACUCCUGCUUAUCACAGC | Rat, Rabbit, Ms | [1123-1141] ORF |
| 823 | UGGCUGUGAUAAGCAGGAG | CUCCUGCUUAUCACAGCCA | Rat, Rabbit, Ms | [1121-1139] ORF |
| 824 | GUGGCUGUGAUAAGCAGGA | UCCUGCUUAUCACAGCCAC | Rat, Rabbit, Ms | [1120-1138] ORF |
| 825 | GGGACAAUACAUUUUUGUC | GACAAAAAUGUAUUGUCCC | | [977-995] ORF |
| 826 | GGAAACCCUCCUAUGACUU | AAGUCAUAGGAGGGUUUCC | Rat, Ms | [804-822] ORF |
| 827 | CUGUGCAUAAUAUAACAGU | ACUGUUAUAUUAUGCACAG | | [724-742] ORF |
| 828 | CUGGUAUUACCGGGUUUAU | AUAAACCCGGUAAUACCAG | | [95-113] ORF |
| 829 | UCAUCACGCUGUGCCUCAU | AUGAGGCACAGCGUGAUGA | | [556-574] ORF |
| 830 | CUCGAAAGAGAAUAAAGAA | UUCUUUAUUCUCUUUCGAG | | [481-499] ORF |
| 831 | GACUAUGAAGGUUUUUCUU | AAGAAAAACCUUCAUAGUC | | [4105-4123] 3′UTR |
| 832 | UGAGAAUAUUGGCCCUUUC | GAAAGGGCCAAUAUUCUCA | | [3771-3789] 3′UTR |
| 833 | UGGAGAGGGUACCUCAGUU | AACUGAGGUACCCUCUCCA | | [3742-3760] 3′UTR |
| 834 | GUCAGGCUCUGUUUAUUGU | ACAAUAAACAGAGCCUGAC | | [3696-3714] 3′UTR |
| 835 | CUGCACCUCAGGUAUCAAU | AUUGAUACCUGAGGUGCAG | | [3638-3656] 3′UTR |
| 836 | CUCUGCGAUUCACACCAUU | AAUGGUGUGAAUCGCAGAG | | [347-365] ORF |
| 837 | GAACUCAGUUGUUCUUUGU | ACAAAGAACAACUGAGUUC | | [3475-3493] 3′UTR |
| 838 | GUCUCAUAUUGGUCAUUCC | GGAAUGACCAAUAUGAGAC | | [3293-3311] 3′UTR |
| 839 | CUAAAUCACUGUUAGUGUG | CACACUAACAGUGAUUUAG | | [3026-3044] 3′UTR |
| 840 | UCACACUCAAUAGUAUGUU | AACAUACUAUUGAGUGUGA | | [2961-2999] 3′UTR |
| 841 | CACACAAAAUGGAUCGCAU | AUGCGAUCCAUUUUGUGUG | | [2871-2889] 3′UTR |
| 842 | GGAAUCUGAUUCUCCCUAG | CUAGGGAGAAUCAGAUUCC | | [2559-2577] 3′UTR |
| 843 | CCAAAGGAAUCUGAUUCUC | GAGAAUCAGAUUCCUUUGG | | [2564-2572] 3′UTR |
| 844 | CAACACUUAUUAGUGACAG | CUGUCACUAAUAAGUGUUG | | [2277-2295] 3′UTR |
| 845 | UCAACACUUAUUAGUGACA | UGUCACUAAUAAGUGUUGA | | [2276-2294] 3′UTR |
| 846 | GUGCUGCUCAACAAGAGUU | AACUCUUGUUGAGCAGCAC | | [266-284] ORF |
| 847 | CCCAUCUUACUCCAGGUUU | AAACCUGGAGUAAGAUGGG | | [2074-2092] 3′UTR |
| 848 | UAAUGGUUUUCCCUUAAAG | CUUUAAGGGAAAACCAUUA | | [1841-1859] 3′UTR |
| 849 | CCUAAUACCAGAAUAGGAG | CUCCUAUUCUGGUAUUAGG | Rabbit | [1596-1614] ORF |
| 850 | UUGCAAGUCAACACCCUAA | UUAGGGUGUUGACUUGCAA | Rabbit | [1582-1600] ORF |
| 851 | GCCAAUCACUUUGCUGUGC | GCACAGCAAAGUGAUUGGC | Rat, Rabbit, Ms | [1476-1494] ORF |
| 852 | UCAGGCCAAUCACUUUGCU | AGCAAAGUGAUUGGCCUGA | Rat, Ms | [1472-1490] ORF |
| 853 | GGCUGUGAUAAGCAGGAGU | ACUCCUGCUUAUCACAGCC | Rat, Rabbit, Ms | [1122-1140] ORF |
| 854 | AGUGCCCAAAGGUGUCCAA | UUGGACACCUUUGGGCACU | | [997-1015] ORF |
| 855 | CCUCCUAUGACUUGGAAAU | AUUUCCAAGUCAUAGGAGG | Rat, Ms | [810-828] ORF |
| 856 | ACCCUCCUAUGACUUGGAA | UUCCAAGUCAUAGGAGGGU | Rat, Ms | [808-826] ORF |
| 857 | UCUGGUAUUACCGGGUUUA | UAAACCCGGUAAUACCAGA | | [94-112] ORF |
| 858 | AUCACGCUGUGCCUCAUAU | AUAUGAGGCACAGCGUGAU | | [558-576] ORF |
| 859 | AUCUCAAUUUUGCUCGAAA | UUUCGAGCAAAAUUGAGAU | | [469-487] ORF |
| 860 | UUCUGAUCCUUAUUCAGUA | UACUGAAUAAGGAUCAGAA | | [413-431] ORF |
| 861 | CUGCUUUUGCAAUUGUGUU | AACACAAUUGCAAAAGCAG | | [4131-4149] 3′UTR |
| 862 | UGAAGGUUUUUCUUAGUUC | GAACUAAGAAAAACCUUCA | | [4110-4128] 3′UTR |
| 863 | CCUGUGUUAUCCAGUUAUG | CAUAACUGGAUAACACAGG | | [3897-3915] 3′UTR |
| 864 | CCAGCUUCAUUUGUUGCUC | GAGCAACAAAUGAAGCUGG | | [3812-3830] 3′UTR |
| 865 | CACAUCUAUUUAAUGUGGA | UCCACAUUAAAUAGAUGUG | | [367-385] ORF |
| 866 | GGGUACCUCAGUUAUAAGG | CCUUAUAACUGAGGUACCC | | [3748-3766] 3′UTR |
| 867 | GAGAGGGUACCUCAGUUAU | AUAACUGAGGUACCCUCUC | | [3744-3762] 3′UTR |
| 868 | CACCUCAGGUAUCAAUUCA | UGAAUUGAUACCUGAGGUG | | [3641-3659] 3′UTR |
| 869 | GCACCUCAGGUAUCAAUUC | GAAUUGAUACCUGAGGUGC | | [3640-3658] 3′UTR |
| 870 | UCACACUGCACCUCAGGUA | UACCUGAGGUGCAGUGUGA | | [3633-3651] 3′UTR |
| 871 | ACUCUGCGAUUCACACCAU | AUGGUGUGAAUCGCAGAGU | | [346-364] ORF |
| 872 | AGAACUCAGUUGUUCUUUG | CAAAGAACAACUGAGUUCU | | [3474-3492] 3′UTR |
| 873 | GGUCACAAAUUUUGAUGCU | AGCAUCAAAAUUUGUGACC | | [3418-3436] 3′UTR |
| 874 | CUGGGAACUAAAACCAGUU | AACUGGUUUUAGUUCCCAG | | [3090-3108] 3′UTR |
| 875 | GGCAUGGAUGAUUGCACUU | AAGUGCAAUCAUCCAUGCC | | [326-344] ORF |
| 876 | GAGAGAGUCUCUAAAUCAC | GUGAUUUAGAGACUCUCUC | | [3016-3034] 3′UTR |
| 877 | CUGAAUAUAUGUUCAAGAG | CUCUUGAACAUAUAUUCAG | | [3000-3018] 3′UTR |
| 878 | ACACUCAAUAGUAUGUUCU | AGAACAUACUAUUGAGUGU | | [2983-3001] 3′UTR |
| 879 | CUCUAUACAGAGAUCAAAU | AUUUGAUCUCUGUAUAGAG | | [2962-2980] 3′UTR |
| 880 | CCUAAAAUUAACAACCCAG | CUGGGUUGUUAAUUUUAGG | | [2933-2951] 3′UTR |
| 881 | GGAUCGCAUCUGUGUGACU | AGUCACACAGAUGCGAUCC | | [2881-2899] 3′UTR |
| 882 | UCCGUAAAUACACACACAA | UUGUGUGUGUAUUUACGGA | | [2859-2877] 3′UTR |
| 883 | GGUGGAGAAUUAACCUCCU | AGGAGGUUAAUUCUCCACC | | [2667-2685] 3′UTR |
| 884 | GCACAUUCUUAUUUGAAGC | GCUUCAAAUAAGAAUGUGC | | [2629-2647] 3′UTR |
| 885 | GACAACUGGACAGGAAUCU | AGAUUCCUGUCCAGUUGUC | | [289-307] ORF |
| 886 | GAAUGAAUUUAGAACCACA | UGUGGUUCUAAAUUCAUUC | | [2460-2478] 3′UTR |
| 887 | GAGAGCGGAUACUCAAGUA | UACUUGAGUAUCCGCUCUC | | [2424-2442] 3′UTR |
| 888 | GGGCCAGAUGUUCUUUCUA | UAGAAAGAACAUCUGGCCC | | [2343-2361] 3′UTR |
| 889 | GUGACAGUAUUGACAUCUG | CAGAUGUCAAUACUGUCAC | | [2289-2307] 3′UTR |
| 890 | GAGUGUCUCAACACUUAUU | AAUAAGUGUUGAGACACUC | | [2269-2287] 3′UTR |
| 891 | CUGCUCAACAAGAGUUCGA | UCGAACUCUUGUUGAGCAG | | [269-287] ORF |
| 892 | GAAACCAAGGAGUAGCUAU | AUAGCUACUCCUUGGUUUC | | [2165-2183] 3′UTR |
| 893 | AGGUUCAGUAACUUCCCUA | UAGGGAAGUUACUGAACCU | | [2028-2046] 3′UTR |
| 894 | CUCUAUGGUUUUGAGAGCA | UGCUCUCAAAACCAUAGAG | | [1906-1924] 3′UTR |
| 895 | CCCUUAAAGGAAUGUCAAA | UUUGACAUUCCUUUAAGGG | | [1851-1869] 3′UTR |
| 896 | GUGGGUUGUGCUGCCAAAU | AUUUGGCAGCACAACCCAC | | [1754-1772] 3′UTR |
| 897 | CUCUUCCAUGAGGAAAUAA | UUAUUUCCUCAUGGAAGAG | | [1733-1751] 3′UTR |
| 898 | GGCCUGAAACAAAAGACUU | AAGUCUUUUGUUUCAGGCC | | [1524-1542] ORF |
| 899 | GCAGCCUGCCUGAAUUUCA | UGAAAUUCAGGCAGGCUGC | Rat, Ms | [183-201] ORF |
| 900 | UCACCAAGGUGGUCACUCA | UGAGUGACCACCUUGGUGA | Rabbit | [904-922] ORF |
| 901 | GUAUCUCUGUGAGAGGUUG | CAACCUCUCACAGAGAUAC | | [851-869] ORF |
| 902 | CUGUAUCUCUGUGAGAGGU | ACCUCUCACAGAGAUACAG | | [849-867] ORF |
| 903 | UGGAAACCCUCCUAUGACU | AGUCAUAGGAGGGUUUCCA | Ms | [803-821] ORF |
| 904 | CCCAAUCCCUCAGUUUGCU | AGCAAACUGAGGGAUUGGG | | [785-803] ORF |
| 905 | UCAUUGGCCUUGCCAUCCA | UGGAUGGCAAGGCCAAUGA | | [661-679] ORF |
| 906 | AGAGGGUACCUCAGUUAUA | UAUAACUGAGGUACCCUCU | | [3745-3763] 3′UTR |
| 907 | UGCACAUCUAUUUAAUGUG | CACAUUAAAUAGAUGUGCA | | [365-383] ORF |
| 908 | ACCAGGAUGGUUUCUGCUU | AAGCAGAAACCAUCCUGGU | | [3591-3609] 3′UTR |
| 909 | GCGAUUCACACCAUUGCAC | GUGCAAUGGUGUGAAUCGC | | [351-369] ORF |
| 910 | CUAUUCCUUUCGUCUCCCA | UGGGAGACGAAAGGAAUAG | | [3501-3519] 3′UTR |
| 911 | UACACAUACUGAUGUUGUU | AACAACAUCAGUAUGUGUA | | [3365-3383] 3′UTR |
| 912 | GGUAUACACAUACUGAUGU | ACAUCAGUAUGUGUAUACC | | [3361-3379] 3′UTR |
| 913 | GGGCAGAAGACUGGUUUAG | CUAAACCAGUCUUCUGCCC | | [3193-3211] 3′UTR |
| 914 | GAAGGGCAGAAGACUGGUU | AACCAGUCUUCUGCCCUUC | | [3190-3208] 3′UTR |
| 915 | CUCCCAUUUCUGGGAACUA | UAGUUCCCAGAAAUGGGAG | | [3081-3099] 3′UTR |
| 916 | AGAGAGAGUCUCUAAAUCA | UGAUUUAGAGACUCUCUCU | | [3015-3033] 3′UTR |
| 917 | UCUCUAUACAGAGAUCAAA | UUUGAUCUCUGUAUAGAGA | | [2961-2979] 3′UTR |
| 918 | CCCAGAAACAAAAAUCUCU | AGAGAUUUUUGUUUCUGGG | | [2947-2965] 3′UTR |
| 919 | ACAAAAUGGAUCGCAUGUG | CAGAUGCGAUCCAUUUUGU | | [2874-2892] 3′UTR |
| 920 | GACUCUGGCUCAUCUAGUC | GACUAGAUGAGCCAGAGUC | | [2691-2709] 3′UTR |
| 921 | CACAUUCUUAUUUGAAGCA | UGCUUCAAAUAAGAAUGUG | | [2630-2648] 3′UTR |

TABLE F
21-mer ligomers NOX2 (NADPH oxidase 2; CYBB)

| | | | | |
|---|---|---|---|---|
| | | | Other | Human-6996020 |
| Number | Sense siRNA | AntiSense siRNA | Species | ORF: 15-1727 |

| | | | | |
|---|---|---|---|---|
| 1 | GGGUUUUCUUUUUGUUCUUAG | CUAAGAACAAAAAGAAAACCC | | [3055-3075] 3′UTR |
| 2 | CAGGGUUUUCUUUUUGUUCUU | AAGAACAAAAAGAAAACCCUG | | [3053-3073] 3′UTR |
| 3 | GCUGAAACCCUGAGUAAACAA | UUGUUUACUCAGGGUUUCAGC | | [1641-1861] ORF |
| 4 | GGGAUAAUGAAUUCAAGACAA | UUGUCUUGAAUUCAUUAUCCC | | [1561-1581] ORF |
| 5 | CGAAUAUAGCCUUUCUGAAAU | AUUUCAGAAAGGCUAUAUUCG | | [3568-3588] 3′UTR |
| 6 | CCACAAAUGUUUAGAACUCUU | AAGAGUUCUAAACAUUUGUGG | | [3131-3151] 3′UTR |
| 7 | GCCCUUAUGAAUAUUGAACUU | AAGUUCAAUAUUCAUAAGGGC | | [2774-2794] 3′UTR |
| 8 | CAACUGGGAUAAUGAAUUCAA | UUGAAUUCAUUAUCCCAGUUG | | [1556-1576] ORF |
| 9 | GCGAAUAUAGCCUUUCUGAAA | UUUCAGAAAGGCUAUAUUCGC | | [3567-3587] 3′UTR |
| 10 | CCAACUGGGAUAAUGAAUUCA | UGAAUUCAUUAUCCCAGUUGG | | [1555-1575] ORF |
| 11 | GCUGUGCCUCAUAUUAAUUAU | AUAAUUAAUAUGAGGCACAGC | | [563-583] ORF |
| 12 | GCAUAAUUAAAACCAGCUUCA | UGAAGCUGGUUUUAAUUAUGC | | [3800-3820] 3′UTR |
| 13 | GGAAAUAAGGAGAAAAGAGAA | UUCUCUUUUCUCCUUAUUUCC | | [3220-3240] 3′UTR |
| 14 | GAACCACACAAUGCCAAGAUA | UAUCUUGGCAUUGUGUGGUUC | | [2471-2491] 3′UTR |
| 15 | GUCAACACCCUAAUACCAGAA | UUCUGGUAUUAGGGUGUUGAC | Rabbit | [1588-1608] ORF |
| 16 | GUGCCUCAUAUUAAUUAUCAC | GUGAUAAUUAAUAUGAGGCAC | | [568-586] ORF |
| 17 | GCAGGCUUUGUAUGUGAAUAA | UUAUUCACAUACAAAGCCUGC | | [4168-4188] 3′UTR |
| 18 | CCCAGAUUUCUUAAAGUUUGA | UCAAACUUUAAGAAAUCUGGG | | [3316-3336] 3′UTR |
| 19 | CCACACAAUGCCAAGAUAGAA | UUCUAUCUUGGCAUUGUGUGG | | [2474-2494] 3′UTR |
| 20 | AGAACCACACAAUGCCAAGAU | AUCUUGGCAUUGUGUGGUUCU | | [2470-2490] 3′UTR |
| 21 | CCAAAAGAAGAAGGAAACCAA | UUGGUUUCCUUCUUCUUUUGG | | [2152-2172] 3′UTR |
| 22 | ACGCUGUGCCUCAUAUUAAUU | AAUUAAUAUGAGGCACAGCGU | | [561-581] ORF |
| 23 | CUUAAAACUGUGCUCAACUAU | AUAGUUGAGCACAGUUUUAAG | | [4228-4248] 3′UTR |
| 24 | GGAAUUCUUAAAACUGUGCUC | GAGCACAGUUUUAAGAAUUCC | | [4222-4242] 3′UTR |
| 25 | CAGGUAUCAAUUCAUCUAUUC | GAAUAGAUGAAUUGAUACCUG | | [3646-3668] 3′UTR |
| 26 | CUCAGGUAUCAAUUCAUCUAU | AUAGAUGAAUUGAUACCUGAG | | [3644-3664] 3′UTR |
| 27 | GAAUAUAGCCUUUCUGAAAUG | CAUUUCAGAAAGGCUAUAUUC | | [3569-3589] 3′UTR |
| 28 | GGGACUAGGAUGAACUAAAUA | UAUUUAGUUCAUCCUAGUCCC | | [3463-3473] 3′UTR |
| 29 | GGAAAAGGAAAUAAGGAGAAA | UUUCUCCUUAUUUCCUUUUCC | | [3214-3234] 3′UTR |
| 30 | GGAGCCACAAAUGUUUAGAAC | GUUCUAAACAUUUGUGGCUCC | | [3127-3147] 3′UTR |
| 31 | GAGCAGGGUUUUCUUUUUGUU | AACAAAAAGAAAACCCUGCUC | | [3050-3070] 3′UTR |
| 32 | CUGUGUGACUAAUGGUUUAUU | AAUAAACCAUUAGUCACACAG | | [2890-2910] 3′UTR |
| 33 | GGCUCUGUUUAUUGUUUCAAU | AUUGAAACAAUAAACAGAGCC | | [3700-3720] 3′UTR |
| 34 | CAGUUGUUCUUUGUCAUACUA | UAGUAUGACAAAGAACAACUG | | [3480-3500] 3′UTR |
| 35 | GGAGAGUGAGAGAAAAUAAAA | UUUUAUUUUCUCUCACUCUCC | | [3243-3263] 3′UTR |
| 36 | GCCACAAAUGUUUAGAACUCU | AGAGUUCUAAACAUUUGUGGC | | [3130-3150] 3′UTR |
| 37 | GCAGGGUUUUCUUUUUGUUCU | AGAACAAAAAGAAAACCCUGC | | [3052-3072] 3′UTR |
| 38 | CCAAGAGCAGGGUUUUCUUUU | AAAAGAAAACCCUGCUCUUGG | | [3046-3066] 3′UTR |
| 39 | ACACAAUGCCAAGAUAGAAUU | AAUUCUAUCUUGGCAUUGUGU | | [2476-2498] 3′UTR |
| 40 | GGAUUGAUUGGAGUUAAUGUA | UACAUUAACUCCAAUCAAUCC | | [2374-2394] 3′UTR |
| 41 | CUGAAACCCUGAGUAAACAAA | UUUGUUUACUCAGGGUUUCAG | | [1642-1662] ORF |
| 42 | UGGGAUAAUGAAUUCAAGACA | UGUCUUGAAUUCAUUAUCCCA | | [1560-1580] ORF |
| 43 | CGCUGUGCCUCAUAUUAAUUA | UAAUUAAUAUGAGGCACAGCG | | [562-582] ORF |
| 44 | GCCCGAGUCAAUAAUUCUGAU | AUCAGAAUUAUUGACUCGGGC | | [399-419] ORF |
| 45 | GGGAAUUCUUAAAACUGUGCU | AGCACAGUUUUAAGAAUUCCC | | [4221-4241] 3′UTR |
| 46 | CCAACUAAGUUAUGAAAGUAG | CUACUUUCAUAACUUAGUUGG | | [3856-3856] 3′UTR |
| 47 | CUUUCUAACCUAUGUGCAUAA | UUAUGCACAUAGGUUAGAAAG | | [3785-3805] 3′UTR |
| 48 | CCCUUUCUAACCUAUGUGCAU | AUGCACAUAGGUUAGAAAGGG | | [3783-3803] 3′UTR |
| 49 | AGUUAUAAGGAGUCUGAGAAU | AUUCUCAGACUCCUUAUAACU | | [3757-3777] 3′UTR |
| 50 | UCAGUUGUUCUUUGUCAUACU | AGUAUGACAAAGAACAACUGA | | [3479-3499] 3′UTR |
| 51 | UGGAGCCACAAAUGUUUAGAA | UUCUAAACAUUUGUGGCUCCA | | [3126-3146] 3′UTR |
| 52 | UCAUAAAAUGGUGGCAUGGAU | AUCCAUGCCACCAUUUUAUGA | | [314-334] ORF |
| 53 | GUGUGACUAAUGGUUUAUUUG | CAAAUAAACCAUUAGUCACAC | | [2892-2912] 3′UTR |
| 54 | UCUGUGUGACUAAUGGUUUAU | AUAAACCAUUAGUCACACAGA | | [2889-2909] 3′UTR |
| 55 | CACAAUGCCAAGAUAGAAUUA | UAAUUCUAUCUUGGCAUUGUG | | [2477-2497] 3′UTR |
| 56 | CUGCCUCAGAAUCCUCAAAAU | AUUUUGAGGAUUCUGAGGCAG | | [2116-2138] 3′UTR |
| 57 | GGUUUGAUACUCUUUCCACAA | UUGUGGAAAGAGUAUCAAACC | | [2088-2108] 3′UTR |
| 58 | GAAAAGAUUCUUGGACUCAAU | AUUGAGUCCAAGAAUCUUUUC | | [1980-2000] 3′UTR |
| 59 | GAGUAAACAAAGCAUCUCCAA | UUGGAGAUGCUUUGUUUACUC | | [1652-1672] ORF |
| 60 | GGAGUUUCAAGAUGCGUGGAA | UUCCACGCAUCUUGAAACUCC | | [1136-1156] ORF |
| 61 | GAAGUGGGACAAUACAUUUUU | AAAAAUGUAUUGUCCCACUUC | | [972-992] ORF |
| 62 | GGUCUUACUUUGAAGUCUUUU | AAAAGACUUCAAAGUAAGACC | | [610-630] ORF |
| 63 | AGGUCUUACUUUGAAGUCUUU | AAAGACUUCAAAGUAAGACCU | | [609-629] ORF |
| 64 | GAGGUCUUACUUUGAAGUCUU | AAGACUUCAAAGUAAGACCUC | | [608-628] ORF |
| 65 | CCGGAGGUCUUACUUUGAAGU | ACUUCAAAGUAAGACCUCCGG | Rabbit | [605-625] ORF |
| 66 | CCAUCCGGAGGUCUUACUUUG | CAAAGUAAGACCUCCGGAUGG | | [601-621] ORF |
| 67 | UAAUUAUCACUUCCUCCACCA | UGGUGGAGGAAGUGAUAAUUA | | [577-597] ORF |
| 68 | CUGUGCCUCAUAUUAAUUAUC | GAUAAUUAAUAUGAGGCACAG | | [584-584] ORF |
| 69 | GCCCUUUCUAACCUAUGUGCA | UGCACAUAGGUUAGAAAGGGC | | [3782-3802] 3′UTR |
| 70 | GGAUGAACUAAAUAAGAACUC | GAGUUCUUAUUUAGUUCAUCC | | [3460-3480] 3′UTR |
| 71 | GGACUAGGAUGAACUAAAUAA | UUAUUUAGUUCAUCCUAGUCC | | [3454-3474] 3′UTR |
| 72 | AGGGACUAGGAUGAACUAAAU | AUUUAGUUCAUCCUAGUCCCU | | [3452-3472] 3′UTR |
| 73 | AGAGCAGGGUUUUCUUUUUGU | ACAAAAAGAAAACCCUGCUCU | | [3049-3069] 3′UTR |
| 74 | CAAGAGCAGGGUUUUCUUUUU | AAAAAGAAAACCCUGCUCUUG | | [3047-3067] 3′UTR |
| 75 | AGAGUCUCUAAAUCACUGUUA | UAACAGUGAUUUAGAGACUCU | | [3019-3039] 3′UTR |
| 76 | CUAGCCCUUAUGAAUAUUGAA | UUCAAUAUUCAUAAGGGGUAG | | [2771-2791] 3′UTR |
| 77 | CCAGCUAGCCCUUAUGAAUAU | AUAUUCAUAAGGGCUAGCUGG | | [2767-2787] 3′UTR |
| 78 | GACUAAUUUCCUGCCCAAAGU | ACUUUGGGCAGGAAAUUAGUC | | [2738-2758] 3′UTR |
| 79 | GCAUGAAAAAAGAGGGUUGGA | UCCAACCCUCUUUUUUCAUGC | | [2646-2666] 3′UTR |
| 80 | UGAAUUUAGAACCACACAAUG | CAUUGUGUGGUUCUAAAUUCA | | [2463-2483] 3′UTR |
| 81 | ACAUAAUACAUGUUGAGAGUG | CACUCUCAACAUGUAUUAUGU | | [2253-2273] 3′UTR |
| 82 | GAAAUUUUCCAGAUCAUUAGG | CCUAAUGAUCUGGAAAAUUUC | | [2232-2252] 3′UTR |
| 83 | CCAGGUUUGAUACUCUUUCCA | UGGAAAGAGUAUCAAACCUGG | | [2065-2105] 3′UTR |
| 84 | GCAUUUCAUUUUCAACAAGGA | UCCUUGUUGAAAAUGAAAUGC | | [1697-1717] ORF |
| 85 | UGAAUUCAAGACAAUUGCAAG | CUUGCAAUUGUCUUGAAUUCA | | [1568-1588] ORF |
| 86 | GGAAAGGAACAAUGCCGGCUU | AAGCCGGCAUUGUUCCUUUCC | | [1412-1432] ORF |
| 87 | CAAGUCAGUCUGGUACAAAUA | UAUUUGUACCAGACUGACUUG | Rabbit | [1274-1294] ORF |
| 88 | GAGUUUCAAGAUGCGUGGAAA | UUUCCACGCAUCUUGAAACUC | | [1137-1157] ORF |
| 89 | GGAAGUGGGACAAUACAUUUU | AAAAUGUAUUGUCCCACUUCC | | [971-991] ORF |
| 90 | AGAGUUUGGCUGUGCAUAAUA | UAUUAUGCACAGCCAAACUCU | | [715-735] ORF |
| 91 | CACGCUGUGCCUCAUAUUAAU | AUUAAUAUGAGGCACAGCGUG | | [560-580] ORF |
| 92 | UGAAUGCCCGAGUCAAUAAUU | AAUUAUUGACUCGGGCAUUCA | | [394-414] ORF |
| 93 | UCUUAAAACUGUGCUCAACUA | UAGUUGAGCACAGUUUUAAGA | | [4227-4247] 3′UTR |
| 94 | UUCUUAAAACUGUGCUCAACU | AGUUGAGCACAGUUUUAAGAA | | [4226-4246] 3′UTR |
| 95 | GAAUUCUUAAAACUGUGCUCA | UGAGCACAGUUUUAAGAAUUC | | [4223-4243] 3′UTR |
| 96 | GCAUUUAUUAACAGGUACUCU | AGAGUACCUGUUAAUAAAUGC | | [4053-4073] 3′UTR |
| 97 | UGAAUAUAAGAGUGCUUGUCA | UGACAAGCACUCUUAUAUUCA | | [3926-3946] 3′UTR |
| 98 | CCAUUGCACAUCUAUUUAAUG | CAUUAAAUAGAUGUGCAAUGG | | [361-381] ORF |
| 99 | ACACCAUUGCACAUCUAUUUA | UAAAUAGAUGUGCAAUGGUGU | | [358-378] ORF |
| 100 | CCUUUCUAACCUAUGUGCAUA | UAUGCACAUAGGUUAGAAAGG | | [3784-3804] 3′UTR |
| 101 | CAGUUAUAAGGAGUCUGAGAA | UUCUCAGACUCCUUAUAACUG | | [3756-3776] 3′UTR |
| 102 | CCUCAGGUAUCAAUUCAUCUA | UAGAUGAAUUGAUACCUGAGG | | [3643-3683] 3′UTR |
| 103 | UGAGGGUAGGUCUGACAAAUA | UAUUUGUCAGACCUACCCUCA | | [3535-3555] 3′UTR |
| 104 | GAAAAGAGAAUGGGAGAGUGA | UCACUCUCCCAUUCUCUUUUC | | [3231-3251] 3′UTR |
| 105 | GAACUCUUCAACUUCGGUAAU | AUUACCGAAGUUGAAGAGUUC | | [3144-3164] 3′UTR |
| 106 | CCUUGGAGCCACAAAUGUUUA | UAAACAUUUGUGGCUCCAAGG | | [3123-3143] 3′UTR |
| 107 | AGCAGGGUUUUCUUUUUGUUC | GAACAAAAAGAAAACCCUGCU | | [3051-3071] 3′UTR |
| 108 | GAACUUAGGAAUUGUGACAAA | UUUGUCACAAUUCCUAAGUUC | | [2789-2809] 3′UTR |
| 109 | CAGCUAGCCCUUAUGAAUAUU | AAUAUUCAUAAGGGCUAGCUG | | [2768-2788] 3′UTR |
| 110 | ACUAAUUUCCUGCCCAAAGUG | CACUUUGGGCAGGAAAUUAGU | | [2739-2759] 3′UTR |
| 111 | GCUCCUUGUGCUAUAAAAUAA | UUAUUUUAUAGCACAAGGAGC | | [2712-2732] 3′UTR |
| 112 | GCUCAUCUAGUCCUGCUCCUU | AAGGAGCAGGACUAGAUGAGC | | [2696-2718] 3′UTR |
| 113 | GGGUUGGAGGUGGAGAAUUAA | UUAAUUCUCCACCUCCAACCC | | [2659-2679] 3′UTR |
| 114 | GAAGCAUGAAAAAAGAGGGUU | AACCCUCUUUUUUCAUGCUUC | | [2643-2663] 3′UTR |
| 115 | GCCAUCAUUAUUAUCAUACUG | CAGUAUGAUAAUAAUGAUGGC | | [2208-2228] 3′UTR |
| 116 | GCUGCCUCAGAAUCCUCAAAA | UUUUGAGGAUUCUGAGGCAGC | | [2115-2135] 3′UTR |
| 117 | GGAUCAAAAGGUUCAGUAACU | AGUUACUGAACCUUUUGAUCC | | [2020-2040] 3′UTR |
| 118 | GGGAGUGCAUUUCAUUUUCAA | UUGAAAAUGAAAUGCACUCCC | | [1691-1711] ORF |
| 119 | GGGCUCUCCAUUUUUGUCAUU | AAUGACAAAAAUGGAGAGCCC | | [39-59] ORF |
| 120 | CGAGCUACAGAUGAAGAAGAA | UUCUUCUUCAUCUGUAGCUCG | | [938-958] ORF |
| 121 | CAAAACCAUCGAGCUACAGAU | AUCUGUAGCUCGAUGGUUUUG | | [929-949] ORF |
| 122 | CGGAGGUCUUACUUUGAAGUC | GACUUCAAAGUAAGACCUCCG | | [606-626] ORF |
| 123 | UCCGGAGGUCUUACUUUGAAG | CUUCAAAGUAAGACCUCCGGA | Rabbit | [804-824] ORF |
| 124 | GACAGGCAAAAUGAAAGUUAU | AUAACUUUCAUUUUGCCUGUC | | [450-470] ORF |
| 125 | AGACAGGCAAAAUGAAAGUUA | UAACUUUCAUUUUGCCUGUCU | | [449-469] ORF |
| 126 | GGAGACAGGCAAAAUGAAAGU | ACUUUCAUUUUGCCUGUCUCC | | [447-467] ORF |
| 127 | CAGGCUUUGUAUGUGAAUAAU | AUUAUUCACAUACAAAGCCUG | | [4169-4189] 3′UTR |
| 128 | UAAGGCCUGCUGUGCGAAUAU | AUAUUCGCACAGCAGGCCUUA | | [3554-3574] 3′UTR |
| 129 | GGAUUAUUCAAGGGACUAGGA | UCCUAGUCCCUUGAAUAAUCC | | [3442-3462] 3′UTR |
| 130 | AGAAUGGGAGAGUGAGAGAAA | UUUCUCUCACUCUCCCAUUCU | | [3237-3257] 3′UTR |
| 131 | AGAAAAGAGAAUGGGAGAGUG | CACUCUCCGAUUCUCUUUUCU | | [3230-3250] 3′UTR |
| 132 | GUAAUGAGGAAGAAGGAGAAA | UUUCUCCUUCUUCCUCAUUAC | | [3160-3180] 3′UTR |
| 133 | GGUAAUGAGGAAGAAGGAGAA | UUCUCCUUCUUCCUCAUUACC | | [3159-3179] 3′UTR |
| 134 | CACAAAUGUUUAGAACUCUUC | GAAGAGUUCUAAACAUUUGUG | | [3132-3152] 3′UTR |
| 135 | CCCUUGGAGCCACAAAUGUUU | AAACAUUUGUGGCUCCAAGGG | | [3122-3142] 3′UTR |
| 136 | CCUUUCAUAAAAUGGUGGCAU | AUGCCACCAUUUUAUGAAAGG | | [310-330] ORF |
| 137 | CACACAAUGCCAAGAUAGAAU | AUUCUAUCUUGGCAUUGUGUG | | [2475-2495] 3′UTR |
| 138 | CCAUCUUACUCCAGGUUUGAU | AUCAAACCUGGAGUAAGAUGG | | [2075-2095] 3′UTR |
| 139 | GAUCAAAAGGUUCAGUAACUU | AAGUUACUGAACCUUUUGAUC | | [2021-2041] 3′UTR |
| 140 | GGAAAAGAUUCUUGGACUCAA | UUGAGUCCAAGAAUCUUUUCC | | [1979-1999] 3′UTR |
| 141 | CUCUUGCCAGUCUGUCGAAAU | AUUUCGACAGACUGGCAAGAG | | [216-236] ORF |
| 142 | UGAGUCUCAGGCCAAUCACUU | AAGUGAUUGGCCUGAGACUCA | | [1466-1486] ORF |
| 143 | CCAGAUGCAGGAAAGGAACAA | UUGUUCCUUUCCUGCAUCUGG | Ms | [1403-1423] ORF |
| 144 | CCUGAAUUUCAACUGCAUGCU | AGCAUGCAGUUGAAAUUCAGG | Rat | [191-211] ORF |
| 145 | UGAGGAAGACUUCUUUAGUAU | AUACUAAAGAAGUCUUCCUCA | | [1052-1072] ORF |
| 146 | UGGAAGUGGGACAAUACAUUU | AAAUGUAUUGUCCCACUUCCA | | [970-990] ORF |
| 147 | UCAAAAUGGAAGUGGGACAAU | AUUGUCCCACUUCCAUUUUGA | | [964-964] ORF |
| 148 | CCACCUAAGUUCUUUUACACA | UGUGUAAAAGAACUUAGGUGG | | [120-140] ORF |
| 149 | UGGUACACACAUCAUCUCUUU | AAAGAGAUGAUGUGUGUACCA | | [630-650] ORF |
| 150 | ACAGGCAAAAUGAAAGUUAUC | GAUAACUUUCAUUUUGCCUGU | | [451-471] ORF |
| 151 | GGCUUUGUAUGUGAAUAAUUC | GAAUUAUUCACAUACAAAGCC | | [4171-4191] 3′UTR |
| 152 | GAAUAUAAGAGUGCUUGUCAU | AUGACAAGCACUCUUAUAUUC | | [3927-3947] 3′UTR |
| 153 | ACCUCAGGUAUCAAUUCAUCU | AGAUGAAUUGAUACCUGAGGU | | [3642-3662] 3′UTR |
| 154 | GAGAAAAGAGAAUGGGAGAGU | ACUCUCCCAUUCUCUUUUCUC | | [3229-3249] 3′UTR |
| 155 | GGAGAAAAGAGAAUGGGAGAG | CUCUCCCAUUCUCUUUUCUCC | | [3228-3248] 3′UTR |
| 156 | AGGAGAAAAGAGAAUGGGAGA | UCUCCCAUUCUCUUUUCUCCU | | [3227-3247] 3′UTR |
| 157 | GAGAGUCUCUAAAUCACUGUU | AACAGUGAUUUAGAGACUCUC | | [3018-3038] 3′UTR |
| 158 | GUCCUGCUCCUUGUGCUAUAA | UUAUAGCACAAGGAGCAGGAC | | [2707-2727] 3′UTR |
| 159 | UGAAGCAUGAAAAAAGAGGGU | ACCCUCUUUUUUCAUGCUUCA | | [2642-2862] 3′UTR |
| 160 | CCAAGGAGUAGCUAUAUAUUU | AAAUAUAUAGCUACUCCUUGG | | [2189-2189] 3′UTR |
| 161 | UGCCUCAGAAUCCUCAAAAUC | GAUUUUGAGGAUUCUGAGGCA | | [2117-2137] 3′UTR |
| 162 | ACUCCAGGUUUGAUACUCUUU | AAAGAGUAUCAAACCUGGAGU | | [2082-2102] 3′UTR |
| 163 | UCAAAAGGGAAAGGAUCAAAA | UUUUGAUCCUUUCCCUUUUGA | | [2008-2028] 3′UTR |
| 164 | GGGAAAAGAUUCUUGGACUCA | UGAGUCCAAGAAUCUUUUCCC | | [1978-1998] 3′UTR |
| 165 | UACAUUUAUGUGGAGCUCUAU | AUAGAGCUCCACAUAAAUGUA | | [1891-1911] 3′UTR |
| 166 | CCCUGCUUAAAAAUGGACAAA | UUUGUCCAUUUUUAAGCAGGG | | [1806-1826] 3′UTR |
| 167 | GGAGUGCAUUUCAUUUUCAAC | GUUGAAAAUGAAAUGCACUCC | | [1692-1712] ORF |
| 168 | GAGUCUCAGGCCAAUCACUUU | AAAGUGAUUGGCCUGAGACUC | | [1467-1487] ORF |
| 169 | GGCACCCUUUUACACUGACAU | AUGUCAGUGUAAAAGGGUGCC | | [1024-1044] ORF |
| 170 | ACAUUUUUGUCAAGUGCCCAA | UUGGGCACUUGACAAAAAUGU | | [985-1005] ORF |
| 171 | GCGGUUUUGGCGAUCUCAACA | UGUUGAGAUCGGCAAAACCGC | | [872-892] ORF |
| 172 | CCCAUGUUUCUGUAUCUCUGU | ACAGAGAUACAGAAACAUGGG | | [840-860] ORF |
| 173 | GGGUCCCAUGUUUCUGUAUCU | AGAUACAGAAACAUGGGACCC | | [836-856] ORF |
| 174 | GAACUUGGAGACAGGCAAAAU | AUUUUGCCUGUCUCCAAGUUC | | [441-461] ORF |
| 175 | GGGACCUGGGAGAUAAUUCUA | UAGAAUUAUCUCCCAGGUCCC | | [4198-4218] 3′UTR |
| 176 | GAAUACUUGCAGGCUUUGUAU | AUACAAAGCCUGCAAGUAUUC | | [4160-4180] 3′UTR |
| 177 | AGGUUUUUCUUAGUUCUUCUG | CAGAAGAACUAAGAAAAACCU | | [4113-4133] 3′UTR |
| 178 | AGGUACUCUAAGAAUGAUGAA | UUCAUCAUUCUUAGAGUACCU | | [4065-4085] 3′UTR |
| 179 | CUAUUUAAUGUGGAAUGGUGU | ACACCAUUCCACAUUAAAUAG | | [372-392] ORF |
| 180 | CGUCUUCCUCUUUGUCUGGUA | UACCAGACAAAGAGGAAGACG | | [80-100] ORF |
| 181 | CUAUCUUCAAAACCAACUAAG | CUUAGUUGGUUUUGAAGAUAG | | [3854-3874] 3′UTR |
| 182 | AGGCUCUGUUUAUUGUUUCAA | UUGAAACAAUAAACAGAGCCU | | [3699-3719] 3′UTR |
| 183 | GCUGUGCGAAUAUAGCCUUUC | GAAAGGCUAUAUUCGCACAGC | | [3562-3582] 3′UTR |
| 184 | AGGCCUGCUGUGCGAAUAUAG | CUAUAUUCGCACAGCAGGCCU | | [3556-3576] 3′UTR |
| 185 | ACUCAGUUGUUCUUUGUCAUA | UAUGACAAAGAACAACUGAGU | | [3477-3497] 3′UTR |
| 186 | UCAAGGGACUAGGAUGAACUA | UAGUUCAUCCUAGUCCCUUGA | | [3449-3469] 3′UTR |
| 187 | UGGGAGAGUGAGAGAAAAUAA | UUAUUUUCUCUCACUCUCCCA | | [3241-3261] 3′UTR |
| 188 | AGAGAAUGGGAGAGUGAGAGA | UCUCUCACUCUCCCAUUCUCU | | [3235-3255] 3′UTR |
| 189 | AGGAAAAGGAAAUAAGGAGAA | UUCUCCUUAUUUCCUUUUCCU | | [3213-3233] 3′UTR |
| 190 | CUAUACAGAGAUCAAAUUCAC | GUGAAUUUGAUCUCUGUAUAG | | [2964-2984] 3′UTR |
| 191 | GCUAGCCCUUAUGAAUAUUGA | UCAAUAUUCAUAAGGGCUAGC | | [2770-2790] 3′UTR |
| 192 | CAGUAUUGACAUCUGAGCAUA | UAUGCUCAGAUGUCAAUACUG | | [2293-2313] 3′UTR |
| 193 | ACCAAGGAGUAGCUAUAUAUU | AAUAUAUAGCUACUCCUUGGU | | [2168-2188] 3′UTR |
| 194 | CAGAAUCCUCAAAAUCAGUUU | AAACUGAUUUUGAGGAUUCUG | | [2122-2142] 3′UTR |
| 195 | CAGUAACUUCCCUAAGAUUAU | AUAAUCUUAGGGAAGUUACUG | | [2033-2053] 3′UTR |
| 196 | AAGGAUCAAAAGGUUCAGUAA | UUACUGAACCUUUUGAUCCUU | | [2018-2038] 3′UTR |
| 197 | GGAAAGGAUCAAAAGGUUCAG | CUGAACCUUUUGAUCCUUUCC | | [2015-2035] 3′UTR |
| 198 | GAAGUUAGGGAAAAGAUUCUU | AAGAAUCUUUUCCCUAACUUC | | [1971-1991] 3′UTR |
| 199 | CUCUCAGUAAUGUCAGUGGAA | UUCCACUGACAUUACUGAGAG | | [1953-1973] 3′UTR |
| 200 | CUAAUACCAGAAUAGGAGUUU | AAACUCCUAUUCUGGUAUUAG | Rabbit | [1597-1617] ORF |
| 201 | GGAUGAGUCUCAGGCCAAUCA | UGAUUGGCCUGAGACUCAUCC | | [1463-1483] ORF |
| 202 | CAAUAACGCCACCAAUCUGAA | UUCAGAUUGGUGGCGUUAUUG | | [1298-1318] ORF |
| 203 | ACCCUUCGCAUCCAUUCUCAA | UUGAGAAUGGAUGCGAAGGGU | | [1256-1276] ORF |
| 204 | GGGUUCAAAAUGGAAGUGGGA | UCCCACUUCCAUUUUGAACCC | | [960-980] ORF |
| 205 | GUGAGAGGUUGGUGCGGUUUU | AAAACCGCACCAACCUCUCAC | | [859-879] ORF |
| 206 | GAACAAAAAAUCUCAGAAUGG | CCAUUCUGAGAUUUUUUGUUC | | [747-767] ORF |
| 207 | AGUUUGGCUGUGCAUAAUAUA | UAUAUUAUGCACAGCCAAACU | | [717-737] ORF |
| 208 | AGAGAGUUUGGCUGUGCAUAA | UUAUGCACAGCCAAACUCUCU | | [713-733] ORF |
| 209 | CUCUUUGUGAUCUUCUUCAUU | AAUGAAGAAGAUCACAAAGAG | | [645-665] ORF |
| 210 | GUCUUUUGGUACACACAUCAU | AUGAUGUGUGUACCAAAAGAC | | [624-644] ORF |
| 211 | GGAGGUCUUACUUUGAAGUCU | AGACUUCAAAGUAAGACCUCC | | [607-627] ORF |
| 212 | GCAUCACUGGAGUUGUCAUCA | UGAUGACAACUCCAGUGAUGC | | [541-561] ORF |
| 213 | GAGACAGGCAAAAUGAAAGUU | AACUUUCAUUUUGCCUGUCUC | | [448-468] ORF |
| 214 | GUGAAUGCCCGAGUCAAUAAU | AUUAUUGACUCGGGCAUUCAC | | [393-413] ORF |
| 215 | CCAAGGUUUUCUAUCUUCAAA | UUUGAAGAUAGAAAACCUUGG | | [3844-3864] 3′UTR |
| 216 | GAGAAUAUUGGCCCUUUCUAA | UUAGAAAGGGCCAAUAUUCUC | | [3772-3792] 3′UTR |
| 217 | AGGGUAGGUCUGACAAAUAAG | CUUAUUUGUCAGACCUACCCU | | [3537-3557] 3′UTR |
| 218 | GACUAGGAUGAACUAAAUAAG | CUUAUUUAGUUCAUCCUAGUC | | [3455-3475] 3′UTR |
| 219 | GGGAGAGUGAGAGAAAAUAAA | UUUAUUUUCUCUCACUCUCCC | | [3242-3262] 3′UTR |
| 220 | GAGUCUCUAAAUCACUGUUAG | CUAACAGUGAUUUAGAGACUC | | [3020-3040] 3′UTR |
| 221 | GAAUAUAUGUUCAAGAGAGAG | CUCUCUCUUGAACAUAUAUUC | | [3002-3022] 3′UTR |
| 222 | GUAUCUGAUAUGGUCAUUUGU | ACAAAUGACCAUAUCAGAUAC | | [2813-2833] 3′UTR |
| 223 | UGAACUUAGGAAUUGUGACAA | UUGUCACAAUUCCUAAGUUCA | | [2788-2808] 3′UTR |
| 224 | AGGGUUCUGCACAUUCUUAUU | AAUAAGAAUGUGCAGAACCCU | | [2621-2641] 3′UTR |
| 225 | CUACAGAAGAAUAUUGGAUUG | CAAUCCAAUAUUCUUCUGUAG | | [2359-2379] 3′UTR |
| 226 | GCCUCAGAAUCCUCAAAAUCA | UGAUUUUGAGGAUUCUGAGGC | | [2118-2138] 3′UTR |
| 227 | UACUCCAGGUUUGAUACUCUU | AAGAGUAUCAAACCUGGAGUA | | [2081-2101] 3′UTR |
| 228 | CCCUAAGAUUAUGAAACUGUG | CACAGUUUCAUAAUCUUAGGG | | [2042-2062] 3′UTR |
| 229 | GUAACUUCCCUAAGAUUAUGA | UCAUAAUCUUAGGGAAGUUAC | | [2035-2055] 3′UTR |
| 230 | AGGGAAAAGAUUCUUGGACUC | GAGUCCAAGAAUCUUUUCCCU | | [1977-1997] 3′UTR |
| 231 | UCAGUGGAAGUUAGGGAAAAG | CUUUUCCCUAACUUCCACUGA | | [1965-1985] 3′UTR |
| 232 | UGAGAGCACUUUUACAAACAU | AUGUUUGUAAAAGUGCUCUCA | | [1917-1937] 3′UTR |
| 233 | UCUAUGGUUUUGAGAGCACUU | AAGUGCUCUCAAAACCAUAGA | | [1907-1927] 3′UTR |
| 234 | GAAAUAAAUGUGGGUUGUGCU | AGCACAACCCACAUUUAUUUC | | [1745-1765] 3′UTR |
| 235 | GUGCAUUUCAUUUUCAACAAG | CUUGUUGAAAAUGAAAUGCAC | | [1695-1715] ORF |
| 236 | UGAUCACAGGCCUGAAACAAA | UUUGUUUCAGGCCUGUGAUCA | | [1516-1536] ORF |
| 237 | UGAUGAGGAGAAAGAUGUGAU | AUCACAUCUUUCUCCUCAUCA | Rabbit, Ms | [1499-1519] ORF |
| 238 | CAGAUGCAGGAAAGGAACAAU | AUUGUUCCUUUCCUGCAUCUG | Ms | [1404-1424] ORF |
| 239 | GCAUCCAUUCUCAAGUCAGUC | GACUGACUUGAGAAUGGAUGC | | [1263-1283] ORF |
| 240 | GCAGGAGUUUCAAGAUGCGUG | CACGCAUCUUGAAACUCCUGC | | [1133-1153] ORF |
| 241 | GCACCCUUUUACACUGACAUC | GAUGUCAGUGUAAAAGGGUGC | | [1025-1045] ORF |
| 242 | CAAAAUGGAAGUGGGACAAUA | UAUUGUCCCACUUCCAUUUUG | | [965-985] ORF |
| 243 | CACUCACCCUUUCAAAACCAU | AUGGUUUUGAAAGGGUGAGUG | Rabbit | [917-937] ORF |
| 244 | UCACUCACCCUUUCAAAACCA | UGGUUUUGAAAGGGUGAGUGA | Rabbit | [916-936] ORF |
| 245 | UCCACCUAAGUUCUUUUACAC | GUGUAAAAGAACUUAGGUGGA | | [119-139] ORF |
| 246 | GGUUUUGGCGAUCUCAACAGA | UCUGUUGAGAUCGCCAAAACC | | [874-894] ORF |
| 247 | CGGUUUUGGCGAUCUCAACAG | CUGUUGAGAUCGCCAAAACCG | | [873-893] ORF |
| 248 | UGUGAGAGGUUGGUGCGGUUU | AAACCGCACCAACCUCUCACA | | [858-878] ORF |
| 249 | GUGAUCUUCUUCAUUGGCCUU | AAGGCCAAUGAAGAAGAUCAC | | [651-671] ORF |
| 250 | GGUACACACAUCAUCUCUUUG | CAAAGAGAUGAUGUGUGUACC | | [631-651] ORF |
| 251 | CUGAACUUGGAGACAGGCAAA | UUUGCCUGUCUCCAAGUUCAG | | [439-459] ORF |
| 252 | UUCUUAGUUCUUCUGCUUUUG | CAAAAGCAGAAGAACUAAGAA | | [4119-4139] 3′UTR |
| 253 | AGCAUUUAUUAACAGGUACUC | GAGUACCUGUUAAUAAAUGCU | | [4052-4072] 3′UTR |
| 254 | CUAAGUUAUGAAAGUAGAGAG | CUCUCUACUUUCAUAACUUAG | | [3870-3890] 3′UTR |
| 255 | UCCAAGGUUUUCUAUCUUCAA | UUGAAGAUAGAAAACCUUGGA | | [3843-3863] 3′UTR |
| 256 | CACCAUUGCACAUCUAUUUAA | UUAAAUAGAUGUGCAAUGGUG | | [359-379] ORF |
| 257 | GAGGGUAGGUCUGACAAAUAA | UUAUUUGUCAGACCUACCCUC | | [3536-3556] 3′UTR |
| 258 | AACUCAGUUGUUCUUUGUCAU | AUGACAAAGAACAACUGAGUU | | [3476-3496] 3′UTR |
| 259 | CAUCCUAAAAUUAACAACCCA | UGGGUUGUUAAUUUUAGGAUG | | [2930-2950] 3′UTR |
| 260 | AGCUAGCCCUUAUGAAUAUUG | CAAUAUUCAUAAGGGCUAGCU | | [2769-2789] 3′UTR |
| 261 | GGGUUCUGCACAUUCUUAUUU | AAAUAAGAAUGUGCAGAACCC | | [2622-2642] 3′UTR |
| 262 | CGAAGACAACUGGACAGGAAU | AUUCCUGUCCAGUUGUCUUCG | | [285-305] ORF |
| 263 | UCAUCAUUUACCACUGUGCUU | AAGCACAGUGGUAAAUGAUGA | | [2399-2419] 3′UTR |
| 264 | GAAGGAAAUUUUCCAGAUCAU | AUGAUCUGGAAAAUUUCCUUC | | [2228-2248] 3′UTR |
| 265 | GUGUCAUUUUUGCCAUCAUUA | UAAUGAUGGCAAAAAUGACAC | | [2197-2217] 3′UTR |
| 266 | CAAAAGAAGAAGGAAACCAAG | CUUGGUUUCCUUCUUCUUUUG | | [2153-2173] 3′UTR |
| 267 | CAGGUUUGAUACUCUUUCCAC | GUGGAAAGAGUAUCAAACCUG | | [2086-2106] 3′UTR |
| 268 | CCUAAGAUUAUGAAACUGUGA | UCACAGUUUCAUAAUCUUAGG | | [2043-2063] 3′UTR |
| 269 | AAAGGAUCAAAAGGUUCAGUA | UACUGAACCUUUUGAUCCUUU | | [2017-2037] 3′UTR |
| 270 | CAAAAGGGAAAGGAUCAAAAG | CUUUUGAUCCUUUCCCUUUUG | | [2009-2029] 3′UTR |
| 271 | GGAAGUUAGGGAAAAGAUUCU | AGAAUCUUUUCCCUAACUUCC | | [1970-1990] 3′UTR |
| 272 | CAGUAAUGUCAGUGGAAGUUA | UAACUUCCACUGACAUUACUG | | [1957-1977] 3′UTR |
| 273 | GAGAGCACUUUUACAAACAUU | AAUGUUUGUAAAAGUGCUCUC | | [1918-1938] 3′UTR |
| 274 | UUACAUUUAUGUGGAGCUCUA | UAGAGCUCCACAUAAAUGUAA | | [1890-1910] 3′UTR |
| 275 | CCUGCUUAAAAAUGGACAAAA | UUUUGUCCAUUUUUAAGCAGG | | [1807-1827] 3′UTR |
| 276 | GGCUCUCCAUUUUUGUCAUUC | GAAUGACAAAAAUGGAGAGCC | | [46-60] ORF |
| 277 | GUGAUCACAGGCCUGAAACAA | UUGUUUCAGGCCUGUGAUCAC | | [1515-1535] ORF |
| 278 | CAUUCUCAAGUCAGUCUGGUA | UACCAGACUGACUUGAGAAUG | | [1268-1288] ORF |
| 279 | CACCCUUCGCAUCCAUUCUCA | UGAGAAUGGAUGCGAAGGGUG | | [1255-1275] ORF |
| 280 | AGCUAUGAGGUGGUGAUGUUA | UAACAUCACCACCUCAUAGCU | Rat, Rabbit, Ms | [1212-1232] ORF |
| 281 | CCUGAGGAAGACUUCUUUAGU | ACUAAAGAAGUCUUCCUCAGG | | [1050-1070] ORF |
| 282 | UGAUAUUCCACCUAAGUUCUU | AAGAACUUAGGUGGAAUAUCA | | [113-133] ORF |
| 283 | GAGUUUGGCUGUGCAUAAUAU | AUAUUAUGCACAGCCAAACUC | | [716-736] ORF |
| 284 | GAGAGUUUGGCUGUGCAUAAU | AUUAUGCACAGCCAAACUCUC | | [714-734] ORF |
| 285 | ACCGGGUUUAUGAUAUUCCAC | GUGGAAUAUCAUAAACCCGGU | | [103-123] ORF |
| 286 | UCUCUUUGUGAUCUUCUUCAU | AUGAAGAAGAUCACAAAGAGA | Rat, Ms | [644-664] ORF |
| 287 | UCAUCUCUUUGUGAUCUUCUU | AAGAAGAUCACAAAGAGAUGA | | [641-661] ORF |
| 288 | UGAAGUCUUUUGGUACACACA | UGUGUGUACCAAAAGACUUCA | | [620-640] ORF |
| 289 | CACUUCCUCCACCAAAACCAU | AUGGUUUUGGUGGAGGAAGUG | | [584-604] ORF |
| 290 | CCUUAUUCAGUAGCACUCUCU | AGAGAGUGCUACUGAAUAAGG | | [420-440] ORF |
| 291 | GUGUGAAUGCCCGAGUCAAUA | UAUUGACUCGGGCAUUCACAC | | [391-411] ORF |
| 292 | GUCUUCCUCUUUGUCUGGUAU | AUACCAGACAAAGAGGAAGAC | | [81-101] ORF |
| 293 | UUGCAGGCUUUGUAUGUGAAU | AUUCACAUACAAAGCCUGCAA | | [4166-4186] 3′UTR |
| 294 | CUUGCAGGCUUUGUAUGUGAA | UUCACAUACAAAGCCUGCAAG | | [4165-4185] 3′UTR |
| 295 | CUAAGAAUGAUGAAGCAUUGU | ACAAUGCUUCAUCAUUCUUAG | | [4072-4092] 3′UTR |
| 296 | CCUUUUUAUCUCUCAAGCCAC | GUGGCUUGAGAGAUAAAAAGG | | [3959-3979] 3′UTR |
| 297 | GUGUUUCUCCAAGGUUUUCUA | UAGAAAACCUUGGAGAAACAC | | [3836-3856] 3′UTR |
| 298 | CACACCAUUGCACAUCUAUUU | AAAUAGAUGUGCAAUGGUGUG | | [357-377] ORF |
| 299 | CAGGCUCUGUUUAUUGUUUCA | UGAAACAAUAAACAGAGGCUG | | [3696-3716] 3′UTR |
| 300 | GUGUUAUUACUAUGAGUCAGG | CCUGACUCAUAGUAAUAACAC | | [3681-3701] 3′UTR |
| 301 | GUUUCUGCUUAGAGACACUUA | UAAGUGUCUCUAAGCAGAAAC | | [3600-3620] 3′UTR |
| 302 | CACUGAGGGUAGGUCUGACAA | UUGUCAGACCUACCCUCAGUG | | [3532-3552] 3′UTR |
| 303 | GCUCUCCAUUUUUGUCAUUCU | AGAAUGACAAAAAUGGAGAGC | | [41-61] ORF |
| 304 | GGGAUUAUUCAAGGGACUAGG | CCUAGUCCCUUGAAUAAUCCC | | [3441-3461] 3′UTR |
| 305 | CUGGUUUAGGAGGAAAAGGAA | UUCCUUUUCCUCCUAAACCAG | | [3203-3223] 3′UTR |
| 306 | CGGUAAUGAGGAAGAAGGAGA | UCUCCUUCUUCCUCAUUACCG | | [3158-3178] 3′UTR |
| 307 | CCCUUAUUUUCCGUAAAUACA | UGUAUUUACGGAAAAUAAGGG | | [2850-2870] 3′UTR |
| 308 | AGGGUUGGAGGUGGAGAAUUA | UAAUUCUCCACCUCCAACCCU | | [2658-2678] 3′UTR |
| 309 | AGAGGGUUGGAGGUGGAGAAU | AUUCUCCACCUCCAACCCUCU | | [2656-2676] 3′UTR |
| 310 | GGUCAAGAACAGGCUAAGGAU | AUCCUUAGCCUGUUCUUGACC | | [2578-2598] 3′UTR |
| 311 | GAGUUCGAAGACAACUGGACA | UGUCCAGUUGUCUUCGAACUC | | [280-300] ORF |
| 312 | GAAGAAGGAAACCAAGGAGUA | UACUCCUUGGUUUCCUUCUUC | | [2158-2178] 3′UTR |
| 313 | CUCAGAAUCCUCAAAAUCAGU | ACUGAUUUUGAGGAUUCUGAG | | [2120-2140] 3′UTR |
| 314 | CUGUCGAAAUCUGCUGUCCUU | AAGGACAGCAGAUUUCGACAG | | [227-247] ORF |
| 315 | CUAUGGUUUUGAGAGCACUUU | AAAGUGCUCUCAAAACCAUAG | | [1908-1928] 3′UTR |
| 316 | GUGGAGCUCUAUGGUUUUGAG | CUCAAAACCAUAGAGCUCCAC | | [1900-1920] 3′UTR |
| 317 | AGGAAAUAAAUGUGGGUUGUG | CACAACCCACAUUUAUUUCCU | | [1743-1763] 3′UTR |
| 318 | GAGGAAAUAAAUGUGGGUUGU | ACAACCCACAUUUAUUUCCUC | | [1742-1762] 3′UTR |
| 319 | UGAGGAAAUAAAUGUGGGUUG | CAACCCACAUUUAUUUCCUCA | | [1741-1761] 3′UTR |
| 320 | CCAUGAGGAAAUAAAUGUGGG | CCCACAUUUAUUUCCUCAUGG | | [1738-1758] 3′UTR |
| 321 | ACCUGAAGCCUUGGCUGAAAC | GUUUCAGCCAAGGCUUCAGGU | Rabbit | [1628-1648] ORF |
| 322 | GAGGAGAAAGAUGUGAUCACA | UGUGAUCACAUCUUUCUCCUC | Rabbit, Ms | [1503-1523] ORF |
| 323 | CCAUGAUGAGGAGAAAGAUGU | ACAUCUUUCUCCUCAUCAUGG | Rabbit, Ms | [1496-1516] ORF |
| 324 | GAAAGGAACAAUGCCGGCUUC | GAAGCCGGCAUUGUUCCUUUC | | [1413-1433] ORF |
| 325 | GCCAGAUGCAGGAAAGGAACA | UGUUCCUUUCCUGCAUCUGGC | | [1402-1422] ORF |
| 326 | GUCAGUCUGGUACAAAUAUUG | CAAUAUUUGUACCAGACUGAC | Rabbit | [1277-1297] ORF |
| 327 | CAUUUUUGUCAUUCUGGUUUG | CAAACCAGAAUGACAAAAAUG | | [47-67] ORF |
| 328 | UGAAGAUGUGUUCAGCUAUGA | UCAUAGCUGAACACAUCUUCA | Rat, Rabbit, Ms | [1199-1219] ORF |
| 329 | UGCGUGGAAACUACCUAAGAU | AUCUUAGGUAGUUUCCACGCA | | [1148-1168] ORF |
| 330 | AAAAUGGAAGUGGGACAAUAC | GUAUUGUCCCACUUCCAUUUU | | [966-986] ORF |
| 331 | GUUUUGGCGAUCUCAACAGAA | UUCUGUUGAGAUCGCCAAAAC | | [875-895] ORF |
| 332 | GUGGGUCCCAUGUUUCUGUAU | AUACAGAAACAUGGGACCCAC | | [834-854] ORF |
| 333 | CUCUCCAUUUUUGUCAUUCUG | CAGAAUGACAAAAAUGGAGAG | | [42-62] ORF |
| 334 | UGAUCCUUAUUCAGUAGCACU | AGUGCUACUGAAUAAGGAUCA | | [416-436] ORF |
| 335 | UGCCCGAGUCAAUAAUUCUGA | UCAGAAUUAUUGACUCGGGCA | | [398-418] ORF |
| 336 | UGCAGGCUUUGUAUGUGAAUA | UAUUCACAUACAAAGCCUGCA | | [4167-4187] 3′UTR |
| 337 | UGAAUACUUGCAGGCUUUGUA | UACAAAGCCUGCAAGUAUUCA | | [4159-4179] 3′UTR |
| 338 | CUCUAAGAAUGAUGAAGCAUU | AAUGCUUCAUCAUUCUUAGAG | | [4070-4090] 3′UTR |
| 339 | ACUCUAAGAAUGAUGAAGCAU | AUGCUUCAUCAUUCUUAGAGU | | [4069-4089] 3′UTR |
| 340 | ACAGGUACUCUAAGAAUGAUG | CAUCAUUCUUAGAGUACCUGU | | [4063-4083] 3′UTR |
| 341 | UGUGUUAUCCAGUUAUGAGAU | AUCUCAUAACUGGAUAACACA | | [3899-3919] 3′UTR |
| 342 | GCUUCAUUUGUUGCUCCGAGA | UCUCGGAGCAACAAAUGAAGC | | [3815-3835] 3′UTR |
| 343 | UCACACCAUUGCACAUCUAUU | AAUAGAUGUGCAAUGGUGUGA | | [356-376] ORF |
| 344 | CAAGGGACUAGGAUGAACUAA | UUAGUUCAUCCUAGUCCCUUG | | [3450-3470] 3′UTR |
| 345 | GAAUGGGAGAGUGAGAGAAAA | UUUUCUCUCACUCUCCCAUUC | | [3238-3258] 3′UTR |
| 346 | GAGGAAAAGGAAAUAAGGAGA | UCUCCUUAUUUCCUUUUCCUC | | [3212-3232] 3′UTR |
| 347 | GGAGGAAAAGGAAAUAAGGAG | CUCCUUAUUUCCUUUUCCUCC | | [3211-3231] 3′UTR |
| 348 | GAAGACUGGUUUAGGAGGAAA | UUUCCUCCUAAACCAGUCUUC | | [3198-3218] 3′UTR |
| 349 | CUCUUCAACUUCGGUAAUGAG | CUCAUUACCGAAGUUGAAGAG | | [3147-3167] 3′UTR |
| 350 | ACUCUUCAACUUCGGUAAUGA | UCAUUACCGAAGUUGAAGAGU | | [3146-3166] 3′UTR |
| 351 | UGUAUCUGAUAUGGUCAUUUG | CAAAUGACCAUAUCAGAUACA | | [2812-2832] 3′UTR |
| 352 | UCCAGCUAGCCCUUAUGAAUA | UAUUCAUAAGGGCUAGCUGGA | | [2766-2786] 3′UTR |
| 353 | CUCCAGCUAGCCCUUAUGAAU | AUUCAUAAGGGCUAGCUGGAG | | [2765-2785] 3′UTR |
| 354 | GGUUCUGCACAUUCUUAUUUG | CAAAUAAGAAUGUGCAGAACC | | [2623-2643] 3′UTR |
| 355 | CUCAUCAUUUACCACUGUGCU | AGCACAGUGGUAAAUGAUGAG | | [2398-2418] 3′UTR |
| 356 | AUACUCAUCAUUUACCACUGU | ACAGUGGUAAAUGAUGAGUAU | | [2395-2415] 3′UTR |
| 357 | GAAUAUUGGAUUGAUUGGAGU | ACUCCAAUCAAUCCAAUAUUC | | [2387-2387] 3′UTR |
| 358 | AGAAUAUUGGAUUGAUUGGAG | CUCCAAUCAAUCCAAUAUUCU | | [2366-2386] 3′UTR |
| 359 | GCAUACUCCAGUUUACUAAUA | UAUUAGUAAACUGGAGUAUGC | | [2309-2329] 3′UTR |
| 360 | CCAGAUCAUUAGGACAUAAUA | UAUUAUGUCCUAAUGAUCUGG | | [2240-2260] 3′UTR |
| 361 | UGUGUCAUUUUUGCCAUCAUU | AAUGAUGGCAAAAAUGACACA | | [2196-2216] 3′UTR |
| 362 | UCAGAAUCCUCAAAAUCAGUU | AACUGAUUUUGAGGAUUCUGA | | [2121-2141] 3′UTR |
| 363 | CCUCAGAAUCCUCAAAAUCAG | CUGAUUUUGAGGAUUCUGAGG | | [2119-2139] 3′UTR |
| 364 | CUCUUUCCACAAUACUGAGCU | AGCUCAGUAUUGUGGAAAGAG | | [2097-2117] 3′UTR |
| 365 | UCCCUAAGAUUAUGAAACUGU | ACAGUUUCAUAAUCUUAGGGA | | [2041-2061] 3′UTR |
| 366 | GAAAGGAUCAAAAGGUUCAGU | ACUGAACCUUUUGAUCCUUUC | | [2016-2036] 3′UTR |
| 367 | AGUGGAAGUUAGGGAAAAGAU | AUCUUUUCCCUAACUUCCACU | | [1967-1987] 3′UTR |
| 368 | CAGUGGAAGUUAGGGAAAAGA | UCUUUUCCCUAACUUCCACUG | | [1966-1986] 3′UTR |
| 369 | CUCAGUAAUGUCAGUGGAAGU | ACUUCCACUGACAUUACUGAG | | [1955-1975] 3′UTR |
| 370 | UGGUUUUGAGAGCACUUUUAC | GUAAAAGUGCUCUCAAAACCA | | [1911-1931] 3′UTR |
| 371 | CAACAAGGAAAACUUCUAACU | AGUUAGAAGUUUUCCUUGUUG | Ms | [1709-1729] |
| | | | | ORF + 3′UTR |
| 372 | CAGCUACAACAUCUACCUCAC | GUGAGGUAGAUGUUGUAGCUG | | [1436-1456] ORF |
| 373 | CCUCAGCUACAACAUCUACCU | AGGUAGAUGUUGUAGCUGAGG | Rat | [1433-1453] ORF |
| 374 | CUUCCUCAGCUACAACAUCUA | UAGAUGUUGUAGCUGAGGAAG | Rat, Rabbit | [1430-1450] ORF |
| 375 | AGAUGCAGGAAAGGAACAAUG | CAUUGUUCCUUUCCUGCAUCU | Ms | [1405-1425] ORF |
| 376 | CGCAUCCAUUCUCAAGUCAGU | ACUGACUUGAGAAUGGAUGCG | | [1262-1282] ORF |
| 377 | UCGCAUCCAUUCUCAAGUCAG | CUGACUUGAGAAUGGAUGCGA | | [1261-1281] ORF |
| 378 | CAGCUAUGAGGUGGUGAUGUU | AACAUCACCACCUCAUAGCUG | Rat, Rabbit, Ms | [1211-1231] ORF |
| 379 | GAAGAUGUGUUCAGCUAUGAG | CUCAUAGCUGAACACAUCUUC | Rat, Rabbit, Ms | [1200-1220] ORF |
| 380 | GAUAAGCAGGAGUUUCAAGAU | AUCUUGAAACUCCUGCUUAUC | Rat, Rabbit | [1128-1148] ORF |
| 381 | GAGGAAGACUUCUUUAGUAUC | GAUACUAAAGAAGUCUUCCUC | | [1053-1073] ORF |
| 382 | UUCAAAAUGGAAGUGGGACAA | UUGUCCCACUUCCAUUUUGAA | | [963-983] ORF |
| 383 | CACCUAAGUUCUUUUACACAA | UUGUGUAAAAGAACUUAGGUG | | [121-141] ORF |
| 384 | GUCCCAUGUUUCUGUAUCUCU | AGAGAUACAGAAACAUGGGAC | | [838-858] ORF |
| 385 | GGUCCCAUGUUUCUGUAUCUC | GAGAUACAGAAACAUGGGACC | | [837-857] ORF |
| 386 | GUUUAUGAUAUUCCACCUAAG | CUUAGGUGGAAUAUCAUAAAC | | [108-128] ORF |
| 387 | GAAGUCUUUUGGUACACACAU | AUGUGUGUACCAAAAGACUUC | | [621-641] ORF |
| 388 | UGGAGUUGUCAUCACGCUGUG | CACAGCGUGAUGACAACUCCA | | [548-568] ORF |
| 389 | GGCAUCACUGGAGUUGUCAUC | GAUGACAACUCCAGUGAUGCC | | [540-560] ORF |
| 390 | UCUAAGAAUGAUGAAGCAUUG | CAAUGCUUCAUCAUUCUUAGA | | [4071-4091] 3′UTR |
| 391 | CAGGUACUCUAAGAAUGAUGA | UCAUCAUUCUUAGAGUACCUG | | [4064-4084] 3′UTR |
| 392 | CUGAGGGUAGGUCUGACAAAU | AUUUGUCAGACCUACCCUCAG | | [3534-3554] 3′UTR |
| 393 | AGAAGACUGGUUUAGGAGGAA | UUCCUCCUAAACCAGUCUUCU | | [3197-3217] 3′UTR |
| 394 | CAAGGAGUAGCUAUAUAUUUC | GAAAUAUAUAGCUACUCCUUG | | [2170-2190] 3′UTR |
| 395 | GGGAAAGGAUCAAAAGGUUCA | UGAACCUUUUGAUCCUUUCCC | | [2014-2034] 3′UTR |
| 396 | AAGGGAAAGGAUCAAAAGGUU | AACCUUUUGAUCCUUUCCCUU | | [2012-2032] 3′UTR |
| 397 | AUCAAAAGGGAAAGGAUCAAA | UUUGAUCCUUUCCCUUUUGAU | | [2007-2027] 3′UTR |
| 398 | GUGGAAGUUAGGGAAAAGAUU | AAUCUUUUCCCUAACUUCCAC | | [1968-1988] 3′UTR |
| 399 | GUCAGUGGAAGUUAGGGAAAA | UUUUCCCUAACUUCCACUGAC | | [1964-1984] 3′UTR |
| 400 | UGUCAGUGGAAGUUAGGGAAA | UUUCCCUAACUUCCACUGACA | | [1963-1983] 3′UTR |
| 401 | UCAGUAAUGUCAGUGGAAGUU | AACUUCCACUGACAUUACUGA | | [1956-1976] 3′UTR |
| 402 | GGAAAUAAAUGUGGGUUGUGC | GCACAACCCACAUUUAUUUCC | | [1744-1764] 3′UTR |
| 403 | UGCAUUUCAUUUUCAACAAGG | CCUUGUUGAAAAUGAAAUGCA | | [1696-1716] ORF |
| 404 | AGCUACAACAUCUACCUCACU | AGUGAGGUAGAUGUUGUAGCU | | [1437-1457] ORF |
| 405 | GCCUUUGAGUGGUUUGCAGAU | AUCUGCAAACCACUCAAAGGC | | [1362-1382] ORF |
| 406 | AGUCAGUCUGGUACAAAUAUU | AAUAUUUGUACCAGACUGACU | Rabbit | [1276-1296] ORF |
| 407 | CCCUUCGCAUCCAUUCUCAAG | CUUGAGAAUGGAUGCGAAGGG | | [1257-1277] ORF |
| 408 | AGUAUCCAUAUCCGCAUCGUU | AACGAUGCGGAUAUGGAUACU | | [1068-1088] ORF |
| 409 | CUGAGGAAGACUUCUUUAGUA | UACUAAAGAAGUCUUCCUCAG | | [1051-1071] ORF |
| 410 | CAUCUCUUUGUGAUCUUCUUC | GAAGAAGAUCACAAAGAGAUG | Rat, Ms | [642-662] ORF |
| 411 | AGUCUUUUGGUACACACAUCA | UGAUGUGUGUACCAAAAGACU | | [623-643] ORF |
| 412 | AGGCAUCACUGGAGUUGUCAU | AUGACAACUCCAGUGAUGCCU | | [539-559] ORF |
| 413 | UGAACUUGGAGACAGGCAAAA | UUUUGCCUGUCUCCAAGUUCA | | [440-460] ORF |
| 414 | UAAUGUGGAAUGGUGUGUGAA | UUCACACACCAUUCCACAUUA | | [377-397] ORF |
| 415 | UAACAGGUACUCUAAGAAUGA | UCAUUCUUAGAGUACCUGUUA | | [4061-4061] 3′UTR |
| 416 | CUCCAAGGUUUUCUAUCUUCA | UGAAGAUAGAAAACCUUGGAG | | [3842-3862] 3′UTR |
| 417 | UUCUCCAAGGUUUUCUAUCUU | AAGAUAGAAAACCUUGGAGAA | | [3840-3860] 3′UTR |
| 418 | UGUUUCUCCAAGGUUUUCUAU | AUAGAAAACCUUGGAGAAACA | | [3837-3857] 3′UTR |
| 419 | UUCACACCAUUGCACAUCUAU | AUAGAUGUGCAAUGGUGUGAA | | [355-375] ORF |
| 420 | GGUUUAGGAGGAAAAGGAAAU | AUUUCCUUUUCCUCCUAAACC | | [3205-3225] 3′UTR |
| 421 | AGACUGGUUUAGGAGGAAAAG | CUUUUCCUCCUAAAGCAGUCU | | [3200-3220] 3′UTR |
| 422 | GUAAAUACACACACAAAAUGG | CCAUUUUGUGUGUGUAUUUAC | | [2862-2882] 3′UTR |
| 423 | GUUCUGCACAUUCUUAUUUGA | UCAAAUAAGAAUGUGCAGAAC | | [2624-2644] 3′UTR |
| 424 | CCUGAAGGGUUCUGCACAUUC | GAAUGUGCAGAACCCUUCAGG | | [2616-2636] 3′UTR |
| 425 | AGAACAGGCUAAGGAUACUAA | UUAGUAUCCUUAGCCUGUUCU | | [2583-2603] 3′UTR |
| 426 | AGGGUAACUGGGCCAGAUGUU | AACAUCUGGCCCAGUUACCCU | | [2334-2354] 3′UTR |
| 427 | UUGUGUCAUUUUUGCCAUCAU | AUGAUGGCAAAAAUGACACAA | | [2195-2215] 3′UTR |
| 428 | AGGGAAAGGAUCAAAAGGUUC | GAACCUUUUGAUCCUUUCCCU | | [2013-2033] 3′UTR |
| 429 | UAGAAUCAAAAGGGAAAGGAU | AUCCUUUCCCUUUUGAUUCUA | | [2003-2023] 3′UTR |
| 430 | UGUAAUGGUUUUCCCUUAAAG | CUUUAAGGGAAAACCAUUACA | | [1839-1859] 3′UTR |
| 431 | UCAACAAGGAAAACUUCUAAC | GUUAGAAGUUUUCCUUGUUGA | Ms | [1708-1728] |
| | | | | ORF + 3′UTR |
| 432 | UGCCUUUGAGUGGUUUGCAGA | UCUGCAAACCACUCAAAGGCA | Rabbit, Ms | [1361-1381] ORF |
| 433 | AGAUGUGUUCAGCUAUGAGGU | ACCUCAUAGCUGAACACAUCU | Rat, Rsbbit, Ms | [1202-1222] ORF |
| 434 | AGAAAACUUCUUGGGUCAGCA | UGCUGACCCAAGAAGUUUUCU | | [141-161] ORF |
| 435 | GACUUCUUUAGUAUCCAUAUC | GAUAUGGAUACUAAAGAAGUC | | [1059-1079] ORF |
| 436 | UGGCACCCUUUUACACUGACA | UGUCAGUGUAAAAGGGUGCCA | | [1023-1043] ORF |
| 437 | CAAGUGCCCAAAGGUGUCCAA | UUGGACACCUUUGGGCACUUC | | [995-1015] ORF |
| 438 | GGGACAAUACAUUUUUGUCAA | UUGACAAAAAUGUAUUGUCCC | | [977-997] ORF |
| 439 | CCUAUGACUUGGAAAUGGAUA | UAUCCAUUUCCAAGUCAUAGG | Ms | [813-833] ORF |
| 440 | UCUUCCUCUUUGUCUGGUAUU | AAUACCAGACAAAGAGGAAGA | | [82-102] ORF |
| 441 | GGACCUGGGAGAUAAUUCUAC | GUAGAAUUAUCUCCCAGGUCC | | [4199-4219] 3′UTR |
| 442 | GUGCAUAAUUAAAACCAGCUU | AAGCUGGUUUUAAUUAUGCAC | | [3798-3818] 3′UTR |
| 443 | AAGUAUGAACUGGAGAGGGUA | UACCCUCUCCAGUUCAUACUU | | [3732-3752] 3′UTR |
| 444 | ACUAUGAGUCAGGCUCUGUUU | AAACAGAGCCUGACUCAUAGU | | [3689-3709] 3′UTR |
| 445 | GUUUAGGAGGAAAAGGAAAUA | UAUUUCCUUUUCCUCCUAAAC | | [3206-3226] 3′UTR |
| 446 | AAGACUGGUUUAGGAGGAAAA | UUUUCCUCCUAAACCAGUCUU | | [3199-3219] 3′UTR |
| 447 | GGAUCGCAUCUGUGUGACUAA | UUAGUCACACAGAUGCGAUCC | | [2881-2901] 3′UTR |
| 448 | GAAGGGUUCUGCACAUUCUUA | UAAGAAUGUGCAGAACCCUUC | | [2619-2639] 3′UTR |
| 449 | CUAGGGUCAAGAACAGGCUAA | UUAGCCUGUUCUUGACCCUAG | | [2574-2594] 3′UTR |
| 450 | AGAAGAAGGAAACCAAGGAGU | ACUCCUUGGUUUCCUUCUUCU | | [2157-2177] 3′UTR |
| 451 | CUUCCCUAAGAUUAUGAAACU | AGUUUCAUAAUCUUAGGGAAG | | [2039-2059] 3′UTR |
| 452 | GAAAGAUGUGAUCACAGGCCU | AGGCCUGUGAUCACAUCUUUC | Ms | [1508-1528] ORF |
| 453 | GAAAACUUCUUGGGUCAGCAC | GUGCUGACCCAAGAAGUUUUC | Rat | [142-162] ORF |
| 454 | GUGGCACCCUUUUACACUGAC | GUCAGUGUAAAAGGGUGCCAC | | [1022-1042] ORF |
| 455 | GAGCUACAGAUGAAGAAGAAG | CUUCUUCUUCAUCUGUAGCUC | | [939-959] ORF |
| 456 | GUGAAAUUUGAAUACUUGCAG | CUGCAAGUAUUCAAAUUUCAC | | [4151-4171] 3′UTR |
| 457 | UGGUUUAGGAGGAAAAGGAAA | UUUCCUUUUCCUCCCUAAACCA | | [3204-3224] 3′UTR |
| 458 | GGGUCAAGAACAGGCUAAGGA | UCCUUAGCCUGUUCUUGACCC | | [2577-2597] 3′UTR |
| 459 | GUAACUGGGCCAGAUGUUCUU | AAGAACAUCUGGCCCAGUUAC | | [2337-2357] 3′UTR |
| 460 | ACUUUGUGUCAUUUUUGCCAU | AUGGCAAAAAUGACACAAAGU | | [2192-2212] 3′UTR |
| 461 | GUAAUGUCAGUGGAAGUUAGG | CCUAACUUCCACUGACAUUAC | | [1959-1979] 3′UTR |
| 462 | GUAAUGGUUUUCCCUUAAAGG | CCUUUAAGGGAAAACCAUUAC | | [1840-1860] 3′UTR |
| 463 | GGCCCAACUGGGAUAAUGAAU | AUUCAUUAUCCCAGUUGGGCC | | [1552-1572] ORF |
| 464 | CUACAACAUCUACCUCACUGG | CCAGUGAGGUAGAUGUUGUAG | | [1439-1459] ORF |
| 465 | GUGGUGAUGUUAGUGGGAGCA | UGCUCCCACUAACAUCACCAC | Rabbit | [1221-1241] ORF |
| 466 | GCGUGGAAACUACCUAAGAUA | UAUCUUAGGUAGUUUCCACGC | | [1149-1169] ORF |
| 467 | CAAUACAUUUUUGUCAAGUGC | GCACUUGACAAAAAUGUAUUG | | [981-1001] ORF |
| 468 | AGUGGGUCCCAUGUUUCUGUA | UACAGAAACAUGGGACCCACU | | [833-853] ORF |
| 469 | GCUUUUGCAAUUGUGUUUGUG | CACAAACACAAUUGCAAAAGC | | [4133-4153] 3′UTR |
| 470 | CUGGAGAGGGUACCUCAGUUA | UAACUGAGGUACCCUCUCCAG | | [3741-3761] 3′UTR |
| 471 | GCAGACUAAUUUCCUGCCCAA | UUGGGCAGGAAAUUAGUCUGC | | [2735-2755] 3′UTR |
| 472 | CUGCUCCUUGUGCUAUAAAAU | AUUUUAUAGCACAAGGAGCAG | | [2710-2730] 3′UTR |
| 473 | AAGAACAGGCUAAGGAUACUA | UAGUAUCCUUAGCCUGUUCUU | | [2582-2602] 3′UTR |
| 474 | GUCAAGAACAGGCUAAGGAUA | UAUCCUUAGCCUGUUCUUGAC | | [2579-2599] 3′UTR |
| 475 | ACAUCUGAGCAUACUCCAGUU | AACUGGAGUAUGCUCAGAUGU | | [2301-2321] 3′UTR |
| 476 | UCCAGAUCAUUAGGACAUAAU | AUUAUGUCCUAAUGAUCUGGA | | [2239-2259] 3′UTR |
| 477 | CUACUUUGUGUCAUUUUUGCC | GGCAAAAAUGACACAAAGUAG | | [2190-2210] 3′UTR |
| 478 | AGGAAUGUCAAAGAUUGUUUG | CAAACAAUCUUUGACAUUCCU | | [1858-1878] 3′UTR |
| 479 | GCAAGUCAACACCCUAAUACC | GGUAUUAGGGUGUUGACUUGC | Rabbit | [1584-1604] ORF |
| 480 | GAGGUGGUGAUGUUAGUGGGA | UCCCACUAACAUCACCACCUC | Rat, Rabbit, Ms | [1218-1238] ORF |
| 481 | GGAGCUGAACGAAUUGUACGU | ACGUACAAUUCGUUCAGCUCC | Rat, Rabbit, Ms | [681-701] ORF |
| 482 | UGUCUGGUAUUACCGGGUUUA | UAAACCCGGUAAUACCAGACA | | [92-112] ORF |
| 483 | AUUUGAAUACUUGCAGGCUUU | AAAGCCUGCAAGUAUUCAAAU | | [4158-4176] 3′UTR |
| 484 | CUGUGCGAAUAUAGCCUUUCU | AGAAAGGCUAUAUUCGCACAG | | [3563-3583] 3′UTR |
| 485 | CGCAUCUGUGUGACUAAUGGU | ACCAUUAGUCACACAGAUGCG | | [2885-2905] 3′UTR |
| 486 | GCACAUUCUUAUUUGAAGCAU | AUGCUUCAAAUAAGAAUGUGC | | [2629-2649] 3′UTR |
| 487 | UGAUUCUCCCUAGGGUCAAGA | UCUUGACCCUAGGGAGAAUCA | | [2565-2585] 3′UTR |
| 488 | CCUUGGCUGAAACCCUGAGUA | UACUCAGGGUUUCAGCCAAGG | | [1636-1656] ORF |
| 489 | CGGCCCAACUGGGAUAAUGAA | UUCAUUAUCCCAGUUGGGCCG | | [1551-1571] ORF |
| 490 | GGCCAAUCACUUUGCUGUGCA | UGCACAGCAAAGUGAUUGGCC | Rat, Rabbit, Ms | [1475-1495] ORF |
| 491 | CUCAAGUCAGUCUGGUACAAA | UUUGUACCAGACUGACUUGAG | Rabbit | [1272-1292] ORF |
| 492 | UUGUCUGGUAUUACCGGGUUU | AAACCCGGUAAUACCAGACAA | | [91-111] ORF |
| 493 | GUGCGAAUAUAGCCUUUCUGA | UCAGAAAGGCUAUAUUCGCAC | | [3565-3585] 3′UTR |
| 494 | GGAUGAUUGCACUUCACUCUG | CAGAGUGAAGUGCAAUCAUCC | | [331-351] ORF |
| 495 | GGAAUCUCACCUUUCAUAAAA | UUUUAUGAAAGGUGAGAUUCC | | [301-321] ORF |
| 496 | UCCUGCUCCUUGUGCUAUAAA | UUUAUAGCACAAGGAGCAGGA | | [2708-2728] 3′UTR |
| 497 | CAUACUCCAGUUUACUAAUAC | GUAUUAGUAAACUGGAGUAUG | | [2310-2330] 3′UTR |
| 498 | UCAAGACAAUUGCAAGUCAAC | GUUGACUUGCAAUUGUCUUGA | Rabbit | [1573-1593] ORF |
| 499 | CCCAACUGGGAUAAUGAAUUC | GAAUUCAUUAUCCCAGUUGGG | | [1554-1574] ORF |
| 500 | CACCAAUCUGAAGCUCAAAAA | UUUUUGAGCUUCAGAUUGGUG | | [1307-1327] ORF |
| 501 | AUAUUGCAAUAACGCCACCAA | UUGGUGGCGUUAUUGCAAUAU | | [1292-1312] ORF |
| 502 | CUACCUAAGAUAGCGGUUGAU | AUCAACCGCUAUCUUAGGUAG | | [1158-1178] ORF |
| 503 | AGAUGCGUGGAAACUACCUAA | UUAGGUAGUUUCCACGCAUCU | | [1145-1165] ORF |
| 504 | CAUGGAGCUGAACGAAUUGUA | UACAAUUCGUUCAGCUCCAUG | Rat, Rabbit, Ms | [678-698] ORF |
| 505 | CCAAAACCAUCCGGAGGUCUU | AAGACCUCCGGAUGGUUUUGG | | [595-615] ORF |
| 506 | GCACAUCUAUUUAAUGUGGAA | UUCCACAUUAAAUAGAUGUGC | | [366-386] ORF |
| 507 | GGAGGUAUACACAUACUGAUG | CAUCAGUAUGUGUAUACCUCC | | [3358-3378] 3′UTR |
| 508 | GAAGGGCAGAAGACUGGUUUA | UAAACCAGUCUUCUGCCCUUC | | [3190-3210] 3′UTR |
| 509 | UGGGAACUAAAACCAGUUUUA | UAAAACUGGUUUUAGUUCCCA | | [3091-3111] 3′UTR |
| 510 | GCCAUGACUCUGGCUCAUCUA | UAGAUGAGCCAGAGUCAUGGC | | [2686-2706] 3′UTR |
| 511 | CUGGACAGGAAUCUCACCUUU | AAAGGUGAGAUUCCUGUCCAG | | [294-314] ORF |
| 512 | UCGAAGACAACUGGACAGGAA | UUCCUGUCCAGUUGUCUUCGA | Rat, Ms | [284-304] ORF |
| 513 | CCAGUUUACUAAUACAGCAGG | CCUGCUGUAUUAGUAAACUGG | | [2316-2336] 3′UTR |
| 514 | AGCAUACUCCAGUUUACUAAU | AUUAGUAAACUGGAGUAUGCU | | [2308-2328] 3′UTR |
| 515 | CUGAGCAUACUCCAGUUUACU | AGUAAACUGGAGUAUGCUCAG | | [2305-2325] 3′UTR |
| 516 | CAUCUGAGCAUACUCCAGUUU | AAACUGGAGUAUGCUCAGAUG | | [2302-2322] 3′UTR |
| 517 | GGAAAUUUUCCAGAUCAUUAG | CUAAUGAUCUGGAAAAUUUCC | | [2231-2251] 3′UTR |
| 518 | CUUGGCUGAAACCCUGAGUAA | UUACUCAGGGUUUCAGCCAAG | | [1637-1657] ORF |
| 519 | CAACACCCUAAUACCAGAAUA | UAUUCUGGUAUUAGGGUGUUG | Rabbit | [1590-1610] ORF |
| 520 | CAAUUGCAAGUCAACACCCUA | UAGGGUGUUGACUUGCAAUUG | Rabbit | [1579-1599] ORF |
| 521 | UUCAAGACAAUUGCAAGUCAA | UUGACUUGCAAUUGUCUUGAA | Rabbit | [1572-1592] ORF |
| 522 | GCCCAACUGGGAUAAUGAAUU | AAUUCAUUAUCCCAGUUGGGC | | [1553-1573] ORF |
| 523 | ACGGCCCAACUGGGAUAAUGA | UCAUUAUCCCAGUUGGGCCGU | | [1550-1570] ORF |
| 524 | GCCAAUCACUUUGCUGUGCAC | GUGCACAGCAAAGUGAUUGGC | Rat, Rabbit, Ms | [1476-1496] ORF |
| 525 | CGGCUUCCUCAGCUACAACAU | AUGUUGUAGCUGAGGAAGCCG | | [1427-1447] ORF |
| 526 | GGACAAUACAUUUUUGUCAAG | CUUGACAAAAAUGUAUUGUCC | | [978-998] ORF |
| 527 | CCCUUUCAAAACCAUCGAGCU | AGCUCGAUGGUUUUGAAAGGG | | [923-943] ORF |
| 528 | CUGGAAACCCUCCUAUGACUU | AAGUCAUAGGAGGGUUUCCAG | Ms | [802-822] ORF |
| 529 | GCAGAGAGUUUGGCUGUGCAU | AUGCACAGCCAAACUCUCUGC | | [711-731] ORF |
| 530 | UGCUCGAAAGAGAAUAAAGAA | UUCUUUAUUCUCUUUCGAGCA | | [479-499] ORF |
| 531 | GGUACCUCAGUUAUAAGGAGU | ACUCCUUAUAACUGAGGUACC | | [3749-3769] 3′UTR |
| 532 | GCACCUCAGGUAUCAAUUCAU | AUGAAUUGAUACCUGAGGUGC | | [3640-3660] 3′UTR |
| 533 | CUGCUGUGCGAAUAUAGCCUU | AAGGCUAUAUUCGCACAGCAG | | [3560-3580] 3′UTR |
| 534 | GGCCAAGAGCAGGGUUUUCUU | AAGAAAACCCUGCUCUUGGCC | | [3044-3064] 3′UTR |
| 535 | CAAGAGAGAGUCUCUAAAUCA | UGAUUUAGAGACUCUCUCUUG | | [3013-3033] 3′UTR |
| 536 | CCUGCUCCUUGUGCUAUAAAA | UUUUAUAGCACAAGGAGCAGG | | [2709-2729] 3′UTR |
| 537 | GGACAUAAUACAUGUUGAGAG | CUCUCAACAUGUAUUAUGUCC | | [2251-2271] 3′UTR |
| 538 | UGAAGGAAAUUUUCCAGAUCA | UGAUCUGGAAAAUUUCCUUCA | | [2227-2247] 3′UTR |
| 539 | UCAACACCCUAAUACCAGAAU | AUUCUGGUAUUAGGGUGUUGA | Rabbit | [1589-1609] ORF |
| 540 | GGGACACACAUGCCUUUGAGU | ACUCAAAGGCAUGUGUGUCCC | | [1351-1371] ORF |
| 541 | UCAAGUCAGUCUGGUACAAAU | AUUUGUACCAGACUGACUUGA | Rabbit | [1273-1293] ORF |
| 542 | UGUGAUAAGCAGGAGUUUCAA | UUGAAACUCCUGCUUAUCACA | Rat, Rabbit | [1125-1145] ORF |
| 543 | CCCUGAGGAAGACUUCUUUAG | CUAAAGAAGUCUUCCUCAGGG | Rat | [1049-1069] ORF |
| 544 | CGAUCUCAACAGAAGGUGGUC | GACCACCUUCUGUUGAGAUCG | | [882-902] ORF |
| 545 | CCAUGUUUCUGUAUCUCUGUG | CACAGAGAUACAGAAACAUGG | | [841-861] ORF |
| 546 | CCAUGGAGCUGAACGAAUUGU | ACAAUUCGUUCAGCUCCAUGG | Rat, Rabbit, Ms | [677-697] ORF |
| 547 | UCAAUUUUGCUCGAAAGAGAA | UUCUCUUUCGAGCAAAAUUGA | | [472-492] ORF |
| 548 | GCCCUGUGUUAUCCAGUUAUG | CAUAACUGGAUAACACAGGGC | | [3895-3915] 3′UTR |
| 549 | CUGCCCUGUGUUAUCCAGUUA | UAACUGGAUAACACAGGGCAG | | [3893-3913] 3′UTR |
| 550 | GUACCUCAGUUAUAAGGAGUC | GACUCCUUAUAACUGAGGUAC | | [3750-3770] 3′UTR |
| 551 | ACACAUACUGAUGUUGUUUUG | CAAAACAACAUCAGUAUGUGU | | [3366-3386] 3′UTR |
| 552 | AGGAAGAAGGAGAAAGAGCUG | CAGCUCUUUCUCCUUCUUCCU | | [3166-3186] 3′UTR |
| 553 | GGGAACUAAAACCAGUUUUAU | AUAAAACUGGUUUUAGUUCCC | | [3092-3112] 3′UTR |
| 554 | CUUUUUGUUCUUAGAACUGCU | AGCAGUUCUAAGAACAAAAAG | | [3062-3082] 3′UTR |
| 555 | GUUGAACGUCUUCCUCUUUGU | ACAAAGAGGAAGACGUUCAAC | | [74-94] ORF |
| 556 | CAGACUAAUUUCCUGCCCAAA | UUUGGGCAGGAAAUUAGUCUG | | [2736-2756] 3′UTR |
| 557 | CUAGUCCUGCUCCUUGUGCUA | UAGCACAAGGAGCAGGACUAG | | [2704-2724] 3′UTR |
| 558 | CAGGAAUCUCACCUUUCAUAA | UUAUGAAAGGUGAGAUUCCUG | | [299-319] ORF |
| 559 | ACAGGAAUCUCACCUUUCAUA | UAUGAAAGGUGAGAUUCCUGU | | [298-318] ORF |
| 560 | GGAGUUAAUGUAAUACUCAUC | GAUGAGUAUUACAUUAACUCC | | [2383-2403] 3′UTR |
| 561 | GGGCCAGAUGUUCUUUCUACA | UGUAGAAAGAACAUCUGGCCC | | [2343-2363] 3′UTR |
| 562 | AUCUGAGCAUACUCCAGUUUA | UAAACUGGAGUAUGCUCAGAU | | [2303-2323] 3′UTR |
| 563 | UCAACAAGAGUUCGAAGACAA | UUGUCUUCGAACUCUUGUUGA | | [273-293] ORF |
| 564 | UUCCAGAUCAUUAGGACAUAA | UUAUGUCCUAAUGAUCUGGAA | | [2238-2258] 3′UTR |
| 565 | CUGAAGGAAAUUUUCCAGAUC | GAUCUGGAAAAUUUCCUUCAG | | [2226-2246] 3′UTR |
| 566 | CCAAAUGCUCAAAUAAUGCUA | UAGCAUUAUUUGAGCAUUUGG | | [1767-1787] 3′UTR |
| 567 | GGCUGAAACCCUGAGUAAACA | UGUUUACUCAGGGUUUCAGCC | | [1640-1660] ORF |
| 568 | CCAGAAUAGGAGUUUUCCUCU | AGAGGAAAACUCCUAUUCUGG | Rabbit | [1603-1623] ORF |
| 569 | AGAUGUGAUCACAGGCCUGAA | UUCAGGCCUGUGAUCACAUCU | | [1511-1531] ORF |
| 570 | GAAUUUCAACUGCAUGCUGAU | AUCAGCAUGCAGUUGAAAUUC | Rat | [194-214] ORF |
| 571 | CCUUUCAAAACCAUCGAGCUA | UAGCUCGAUGGUUUUGAAAGG | | [924-944] ORF |
| 572 | CAUGUUUCUGUAUCUCUGUGA | UCACAGAGAUACAGAAACAUG | | [842-862] ORF |
| 573 | GAAAAAUAAAGGAAUGCCCAA | UUGGGCAUUCCUUUAUUUUUC | | [769-789] ORF |
| 574 | CGAGUCAAUAAUUCUGAUCCU | AGGAUCAGAAUUAUUGACUCG | | [402-422] ORF |
| 575 | GUUCUUCUGCUUUUGCAAUUG | CAAUUGCAAAAGCAGAAGAAC | | [4125-4145] 3′UTR |
| 576 | UGCCCUGUGUUAUCCAGUUAU | AUAACUGGAUAACACAGGGCA | | [3894-3914] 3′UTR |
| 577 | GUCUGAGAAUAUUGGCCCUUU | AAAGGGCCAAUAUUCUCAGAC | | [3768-3788] 3′UTR |
| 578 | GGAGAGGGUACCUCAGUUAUA | UAUAACUGAGGUACCCUCUCC | | [3743-3763] 3′UTR |
| 579 | UUCUUAGAACUGCUCCCAUUU | AAAUGGGAGCAGUUCUAAGAA | | [3069-3089] 3′UTR |
| 580 | CCGUAAAUACACACACAAAAU | AUUUUGUGUGUGUAUUUACGG | | [2860-2880] 3′UTR |
| 581 | ACUGGACAGGAAUCUCACCUU | AAGGUGAGAUUCCUGUCCAGU | | [293-313] ORF |
| 582 | GAAUGAAUUUAGAACCACACA | UGUGUGGUUCUAAAUUCAUUC | | [2460-2480]3′UTR |
| 583 | GUUUACUAAUACAGCAGGGUA | UACCCUGCUGUAUUAGUAAAC | | [2319-2339] 3′UTR |
| 584 | AGAGUGUCUCAACACUUAUUA | UAAUAAGUGUUGAGACACUCU | | [2268-2288] 3′UTR |
| 585 | UGAGAGUGUCUCAACACUUAU | AUAAGUGUUGAGACACUCUCA | | [2266-2286] 3′UTR |
| 586 | GGAAACCAAGGAGUAGCUAUA | UAUAGCUACUCCUUGGUUUCC | | [2164-2184] 3′UTR |
| 587 | GUUCAGUAACUUCCCUAAGAU | AUCUUAGGGAAGUUACUGAAC | | [2030-2050] 3′UTR |
| 588 | CUCUUCCAUGAGGAAAUAAAU | AUUUAUUUCCUCAUGGAAGAG | | [1733-1753] 3′UTR |
| 589 | CCCUGAGUAAACAAAGCAUCU | AGAUGCUUUGUUUACUCAGGG | | [1648-1668] ORF |
| 590 | UGAAACCCUGAGUAAACAAAG | CUUUGUUUACUCAGGGUUUCA | | [1643-1663] ORF |
| 591 | GACCUGAAGCCUUGGCUGAAA | UUUCAGCCAAGGCUUCAGGUC | Rabbit | [1627-1647] ORF |
| 592 | GUGCACCAUGAUGAGGAGAAA | UUUCUCCUCAUCAUGGUGCAC | Rabbit, Ms | [1491-1511] ORF |
| 593 | CUGCCUGAAUUUCAACUGCAU | AUGCAGUUGAAAUUCAGGCAG | Rat | [188-208] ORF |
| 594 | GUCACACCCUUCGCAUCCAUU | AAUGGAUGCGAAGGGUGUGAC | | [1251-1271] ORF |
| 595 | UCAAUGCUUGUGGCUGUGAUA | UAUCACAGCCACAAGCAUUGA | Rabbit, Ms | [1111-1131] ORF |
| 596 | AGCUGGAGUGGCACCCUUUUA | UAAAAGGGUGCCACUCCAGCU | | [1015-1035] ORF |
| 597 | CCAUCGAGCUACAGAUGAAGA | UCUUCAUCUGUAGCUCGAUGG | | [934-954] ORF |
| 598 | GGGUUUAUGAUAUUCCACCUA | UAGGUGGAAUAUCAUAAACCC | | [106-126] ORF |
| 599 | GCUGUGCAUAAUAUAACAGUU | AACUGUUAUAUUAUGCACAGC | | [723-743] ORF |
| 600 | UUCAUUGGCCUUGCCAUCCAU | AUGGAUGGCAAGGCCAAUGAA | | [660-680] ORF |
| 601 | CAAAACCAUCCGGAGGUCUUA | UAAGACCUCCGGAUGGUUUUG | | [596-616] ORF |
| 602 | CCCUGUGUUAUCCAGUUAUGA | UCAUAACUGGAUAACACAGGG | | [3896-3916]3′UTR |
| 603 | AUAUUGGCCCUUUCUAACCUA | UAGGUUAGAAAGGGCCAAUAU | | [3776-3796] 3′UTR |
| 604 | GGGUACCUCAGUUAUAAGGAG | CUCCUUAUAACUGAGGUACCC | | [3748-3768] 3′UTR |
| 605 | UGCGAAUAUAGCCUUUCUGAA | UUCAGAAAGGCUAUAUUCGCA | | [3566-3586] 3′UTR |
| 606 | UGCUGUGCGAAUAUAGCCUUU | AAAGGCUAUAUUCGCACAGCA | | [3561-3581] 3′UTR |
| 607 | CUGACAAAUAAGGCCUGCUGU | ACAGCAGGCCUUAUUUGUCAG | | [3546-3566] 3′UTR |
| 608 | GGGUCUCAUAUUGGUCAUUCC | GGAAUGACCAAUAUGAGACCC | | [3291-3311]3′UTR |
| 609 | GCAUGGAUGAUUGCACUUCAC | GUGAAGUGCAAUCAUCCAUGC | | [327-347] ORF |
| 610 | GUGGCAUGGAUGAUUGCACUU | AAGUGCAAUCAUCCAUGCCAC | | [324-344] ORF |
| 611 | CUCCCAUUUCUGGGAACUAAA | UUUAGUUCCCAGAAAUGGGAG | | [3081-3101] 3′UTR |
| 612 | AGAAUUAACCUCCUGCCAUGA | UCAUGGCAGGAGGUUAAUUCU | | [2672-2692] 3′UTR |
| 613 | GACAGGAAUCUCACCUUUCAU | AUGAAAGGUGAGAUUCCUGUC | | [297-317] ORF |
| 614 | GGGUUGAACGUCUUCCUCUUU | AAAGAGGAAGACGUUCAACCC | | [72-92] ORF |
| 615 | GGCCAGAUGUUCUUUCUACAG | CUGUAGAAAGAACAUCUGGCC | | [2344-2364] 3′UTR |
| 616 | UCCAGUUUACUAAUACAGCAG | CUGCUGUAUUAGUAAACUGGA | | [2315-2335] 3′UTR |
| 617 | GAGCAUACUCCAGUUUACUAA | UUAGUAAACUGGAGUAUGCUC | | [2307-2327] 3′UTR |
| 618 | GACAUAAUACAUGUUGAGAGU | ACUCUCAACAUGUAUUAUGUC | | [2252-2272] 3′UTR |
| 619 | AGGACAUAAUACAUGUUGAGA | UCUCAACAUGUAUUAUGUCCU | | [2250-2270] 3′UTR |
| 620 | ACUGAAGGAAAUUUUCCAGAU | AUCUGGAAAAUUUCCUUCAGU | | [2225-2245] 3′UTR |
| 621 | GAAACCAAGGAGUAGCUAUAU | AUAUAGCUACUCCUUGGUUUC | | [2165-2185] 3′UTR |
| 622 | AGCCCAUCUUACUCCAGGUUU | AAACCUGGAGUAAGAUGGGCU | | [2072-2092] 3′UTR |
| 623 | CAGGCCUGAAACAAAAGACUU | AAGUCUUUUGUUUCAGGCCUG | | [1522-1542] ORF |
| 624 | CACCAUGAUGAGGAGAAAGAU | AUCUUUCUCCUCAUCAUGGUG | Rabbit, Ms | [1494-1514] ORF |
| 625 | CACUUUGCUGUGCACCAUGAU | AUCAUGGUGCACAGCAAAGUG | Rabbit, Ms | [1482-1502] ORF |
| 626 | CCAAUCACUUUGCUGUGCACC | GGUGCACAGCAAAGUGAUUGG | Rat, Rabbit, Ms | [1477-1497] ORF |
| 627 | GCCACCAAUCUGAAGCUCAAA | UUUGAGCUUCAGAUUGGUGGC | | [1305-1325] ORF |
| 628 | UGGCUGUGAUAAGCAGGAGUU | AACUCCUGCUUAUCACAGCCA | Rat, Rabbit, Ms | [1121-1141] ORF |
| 629 | GUGGUCACUCACCCUUUCAAA | UUUGAAAGGGUGAGUGACCAC | Rabbit | [912-932] ORF |
| 630 | GCGAUCUCAACAGAAGGUGGU | ACCACCUUCUGUUGAGAUCGC | | [881-901] ORF |
| 631 | GUGCGGUUUUGGCGAUCUCAA | UUGAGAUCGCCAAAACCGCAC | | [870-890] ORF |
| 632 | GAAUGCCCAAUCCCUCAGUUU | AAACUGAGGGAUUGGGCAUUC | | [780-800] ORF |
| 633 | GGAAAAAUAAAGGAAUGCCCA | UGGGCAUUCCUUUAUUUUUCC | | [768-788] ORF |
| 634 | AACCAUCCGGAGGUCUUACUU | AAGUAAGACCUCCGGAUGGUU | | [599-619] ORF |
| 635 | ACCAAAACCAUCCGGAGGUCU | AGACCUCCGGAUGGUUUUGGU | | [594-614] ORF |
| 636 | GAGUCAAUAAUUCUGAUCCUU | AAGGAUCAGAAUUAUUGACUC | | [403-423] ORF |
| 637 | CCCGAGUCAAUAAUUCUGAUC | GAUCAGAAUUAUUGACUCGGG | | [400-420] ORF |
| 638 | UGCACAUCUAUUUAAUGUGGA | UCCACAUUAAAUAGAUGUGCA | | [365-385] ORF |
| 639 | UGCAUAAUUAAAACCAGCUUC | GAAGCUGGUUUUAAUUAUGCA | | [3799-3819] 3′UTR |
| 640 | CUGAGAAUAUUGGCCCUUUCU | AGAAAGGGCCAAUAUUCUCAG | | [3770-3790] 3′UTR |
| 641 | UCACUCUGCGAUUCACACCAU | AUGGUGUGAAUCGCAGAGUGA | | [344-364] ORF |
| 642 | CUUUCUGAAAUGUACCAGGAU | AUCCUGGUACAUUUCAGAAAG | | [3578-3598] 3′UTR |
| 643 | CCUUUCUGAAAUGUACCAGGA | UCCUGGUACAUUUCAGAAAGG | | [3577-3597] 3′UTR |
| 644 | GGUCACAAAUUUUGAUGCUGA | UCAGCAUCAAAAUUUGUGACC | | [3418-3438] 3′UTR |
| 645 | AGGUAUACACAUACUGAUGUU | AACAUCAGUAUGUGUAUACCU | | [3360-3380] 3′UTR |
| 646 | GCCAAGAGCAGGGUUUUCUUU | AAAGAAAACCCUGCUCUUGGC | | [3045-3065] 3′UTR |
| 647 | UGUUCAAGAGAGAGUCUCUAA | UUAGAGACUCUCUCUUGAACA | | [3009-3029] 3′UTR |
| 648 | CCCAGAAACAAAAAUCUCUAU | AUAGAGAUUUUUGUUUCUGGG | | [2947-2967] 3′UTR |
| 649 | CCUAAAAUUAACAACCCAGAA | UUCUGGGUUGUUAAUUUUAGG | | [2933-2953] 3′UTR |
| 650 | GCAUCUGUGUGACUAAUGGUU | AACCAUUAGUCACACAGAUGC | | [2886-2906] 3′UTR |
| 651 | GCGGAUACUCAAGUAAGUUUU | AAAACUUACUUGAGUAUCCGC | | [2428-2448] 3′UTR |
| 652 | UGAGCAUACUCCAGUUUACUA | UAGUAAACUGGAGUAUGCUCA | | [2306-2326] 3′UTR |
| 653 | CAAUACUGAGCUGCCUCAGAA | UUCUGAGGCAGCUCAGUAUUG | | [2106-2126] 3′UTR |
| 654 | GUGCUGCCAAAUGCUCAAAUA | UAUUUGAGCAUUUGGCAGCAC | | [1761-1781] 3′UTR |
| 655 | AGAAUAGGAGUUUUCCUCUGU | ACAGAGGAAAACUCCUAUUCU | Rabbit | [1605-1625] ORF |
| 656 | CCCUAAUACCAGAAUAGGAGU | ACUCCUAUUCUGGUAUUAGGG | Rabbit | [1595-1615] ORF |
| 657 | UUGCAAGUCAACACCCUAAUA | UAUUAGGGUGUUGACUUGCAA | Rabbit | [1582-1602] ORF |
| 658 | AGCCUGCCUGAAUUUCAACUG | CAGUUGAAAUUCAGGCAGGCU | Rat, Ms | [185-205] ORF |
| 659 | GCAGCCUGCCUGAAUUUCAAC | GUUGAAAUUCAGGCAGGCUGC | Rat, Ms | [183-203] ORF |
| 660 | CAAUGCUUGUGGCUGUGAUAA | UUAUCACAGCCACAAGCAUUG | Rabbit, Ms | [1112-1132] ORF |
| 661 | CUGGAGUGGCACCCUUUUACA | UGUAAAAGGGUGCCACUCCAG | | [1017-1037] ORF |
| 662 | UCCAUUUUUGUCAUUCUGGUU | AACCAGAAUGACAAAAAUGGA | | [45-65] ORF |
| 663 | ACAGAAGGUGGUCAUCACCAA | UUGGUGAUGACCACCUUCUGU | | [890-910] ORF |
| 664 | AUCUCAACAGAAGGUGGUCAU | AUGACCACCUUCUGUUGAGAU | | [884-904] ORF |
| 665 | AGUUUGCUGGAAACCCUCCUA | UAGGAGGGUUUCCAGCAAACU | | [796-816] ORF |
| 666 | GGCUGUGCAUAAUAUAACAGU | ACUGUUAUAUUAUGCACAGCC | | [722-742] ORF |
| 667 | UCACGCUGUGCCUCAUAUUAA | UUAAUAUGAGGCACAGCGUGA | | [559-579] ORF |
| 668 | CAUCACGCUGUGCCUCAUAUU | AAUAUGAGGCACAGCGUGAUG | | [557-577] ORF |
| 669 | GUCAUCACGCUGUGCCUCAUA | UAUGAGGCACAGCGUGAUGAC | | [555-575] ORF |
| 670 | CGAAAGAGAAUAAAGAACCCU | AGGGUUCUUUAUUCUCUUUCG | | [483-503] ORF |
| 671 | CAAUUUUGCUCGAAAGAGAAU | AUUCUCUUUCGAGCAAAAUUG | | [473-493] ORF |
| 672 | GGCAAAAUGAAAGUUAUCUCA | UGAGAUAACUUUCAUUUUGCC | | [454-474] ORF |
| 673 | CCGAGAGUGUUUCUCCAAGGU | ACCUUGGAGAAACACUCUCGG | | [3830-3850] 3′UTR |
| 674 | AGUCUGAGAAUAUUGGCCCUU | AAGGGCCAAUAUUCUCAGACU | | [3767-3787] 3′UTR |
| 675 | CCUCAGUUAUAAGGAGUCUGA | UCAGACUCCUUAUAACUGAGG | | [3753-3773] 3′UTR |
| 676 | GACACUUAGGUCCAGCCUGUU | AACAGGCUGGACCUAAGUGUC | | [3613-3633] 3′UTR |
| 677 | GUCACAAAUUUUGAUGCUGAG | CUCAGCAUCAAAAUUUGUGAC | | [3419-3439] 3′UTR |
| 678 | GUAUACACAUACUGAUGUUGU | ACAACAUCAGUAUGUGUAUAC | | [3362-3382] 3′UTR |
| 679 | GGUAUACACAUACUGAUGUUG | CAACAUCAGUAUGUGUAUACC | | [3361-3381] 3′UTR |
| 680 | GAAGGAGGUAUACACAUACUG | CAGUAUGUGUAUACCUCCUUC | | [3355-3375] 3′UTR |
| 681 | GGCAUGGAUGAUUGCACUUCA | UGAAGUGCAAUCAUCCAUGCC | | [326-346] ORF |
| 682 | AGAACUCUUCAACUUCGGUAA | UUACCGAAGUUGAAGAGUUCU | | [3143-3163] 3′UTR |
| 683 | CUCACCUUUCAUAAAAUGGUG | CACCAUUUUAUGAAAGGUGAG | | [306-326] ORF |
| 684 | AGGAAUCUCACCUUUCAUAAA | UUUAUGAAAGGUGAGAUUCCU | | [300-320] ORF |
| 685 | AGGUGGAGAAUUAACCUCCUG | CAGGAGGUUAAUUCUCCACCU | | [2666-2686] 3′UTR |
| 686 | GGACAGGAAUCUCACCUUUCA | UGAAAGGUGAGAUUCCUGUCC | | [296-316] ORF |
| 687 | GGAUACUAACCAAUAGGAUUG | CAAUCCUAUUGGUUAGUAUCC | | [2595-2615] 3′UTR |
| 688 | CAACACUUAUUAGUGACAGUA | UACUGUCACUAAUAAGUGUUG | | [2277-2297] 3′UTR |
| 689 | GGAGUAGCUAUAUAUUUCUAC | GUAGAAAUAUAUAGCUACUCC | | [2173-2193] 3′UTR |
| 690 | CCCAUCUUACUCCAGGUUUGA | UCAAACCUGGAGUAAGAUGGG | | [2074-2094] 3′UTR |
| 691 | GUCUCUUCCAUGAGGAAAUAA | UUAUUUCCUCAUGGAAGAGAC | | [1731-1751] 3′UTR |
| 692 | CCACCAAUCUGAAGCUCAAAA | UUUUGAGCUUCAGAUUGGUGG | | [1306-1326] ORF |
| 693 | GGUACAAAUAUUGCAAUAACG | CGUUAUUGCAAUAUUUGUACC | | [1285-1305] ORF |
| 694 | GUCUGGUACAAAUAUUGCAAU | AUUGCAAUAUUUGUACCAGAC | | [1281-1301] ORF |
| 695 | GGAAACUACCUAAGAUAGCGG | CCGCUAUCUUAGGUAGUUUCC | | [1153-1173] ORF |
| 696 | CCAUUUUUGUCAUUCUGGUUU | AAACCAGAAUGACAAAAAUGG | | [46-66] ORF |
| 697 | CUCAGUUUGCUGGAAACCCUC | GAGGGUUUCCAGCAAACUGAG | | [793-813] ORF |
| 698 | GGAAUGCCCAAUCCCUCAGUU | AACUGAGGGAUUGGGCAUUCC | | [779-799] ORF |
| 699 | CGGGUUUAUGAUAUUCCACCU | AGGUGGAAUAUCAUAAACCCG | | [105-125] ORF |
| 700 | GUCUGGUAUUACCGGGUUUAU | AUAAACCCGGUAAUACCAGAC | | [93-113] ORF |
| 701 | UUAGUUCUUCUGCUUUUGCAA | UUGCAAAAGCAGAAGAACUAA | | [4122-4142] 3′UTR |
| 702 | CGAGAGUGUUUCUCCAAGGUU | AACCUUGGAGAAACACUCUCG | | [3831-3851] 3′UTR |
| 703 | UCUGAGAAUAUUGGCCCUUUC | GAAAGGGCCAAUAUUCUCAGA | | [3769-3789] 3′UTR |
| 704 | GAGAGGGUACCUCAGUUAUAA | UUAUAACUGAGGUACCCUCUC | | [3744-3764] 3′UTR |
| 705 | UGGAGAGGGUACCUCAGUUAU | AUAACUGAGGUACCCUCUCCA | | [3742-3762] 3′UTR |
| 706 | CACCAAAGUAUGAACUGGAGA | UCUCCAGUUCAUACUUUGGUG | | [3727-3747] 3′UTR |
| 707 | CUAUGAGUCAGGCUCUGUUUA | UAAACAGAGCCUGACUCAUAG | | [3690-3710] 3′UTR |
| 708 | ACACUGCACCUCAGGUAUCAA | UUGAUACCUGAGGUGCAGUGU | | [3635-3655] 3′UTR |
| 709 | CACUCUGCGAUUCACACCAUU | AAUGGUGUGAAUCGCAGAGUG | | [345-365] ORF |
| 710 | GUCUGACAAAUAAGGCCUGCU | AGCAGGCCUUAUUUGUCAGAC | | [3544-3564] 3′UTR |
| 711 | AGGAGGUAUACACAUACUGAU | AUCAGUAUGUGUAUACCUCCU | | [3357-3377] 3′UTR |
| 712 | GGAAGGGCAGAAGACUGGUUU | AAACCAGUCUUCUGCCCUUCC | | [3189-3209] 3′UTR |
| 713 | GAGCCACAAAUGUUUAGAACU | AGUUCUAAACAUUUGUGGCUC | | [3126-3146] 3′UTR |
| 714 | CACUCAAUAGUAUGUUCUGAA | UUCAGAACAUACUAUUGAGUG | | [2984-3004] 3′UTR |
| 715 | AGAGAUCAAAUUCACACUCAA | UUGAGUGUGAAUUUGAUCUCU | | [2970-2990] 3′UTR |
| 716 | AGCCCUUAUGAAUAUUGAACU | AGUUCAAUAUUCAUAAGGGCU | | [2773-2793] 3′UTR |
| 717 | AGACUAAUUUCCUGCCCAAAG | CUUUGGGCAGGAAAUUAGUCU | | [2737-2757] 3′UTR |
| 718 | UGCAGACUAAUUUCCUGCCCA | UGGGCAGGAAAUUAGUCUGCA | | [2734-2754] 3′UTR |
| 719 | GCCUGAAGGGUUCUGCACAUU | AAUGUGCAGAACCCUUCAGGC | | [2615-2635] 3′UTR |
| 720 | CAGGCUAAGGAUACUAACCAA | UUGGUUAGUAUCCUUAGCCUG | | [2587-2607] 3′UTR |
| 721 | ACUGUCAUAGACCAAAGGAAU | AUUCCUUUGGUCUAUGACAGU | | [2543-2563] 3′UTR |
| 722 | AGAUGUUCUUUCUACAGAAGA | UCUUCUGUAGAAAGAACAUCU | | [2348-2368] 3′UTR |
| 723 | GACAGUAUUGACAUCUGAGCA | UGCUCAGAUGUCAAUACUGUC | | [2291-2311] 3′UTR |
| 724 | GUGACAGUAUUGACAUCUGAG | CUCAGAUGUCAAUACUGUCAC | | [2289-2309] 3′UTR |
| 725 | GCUGCUCAACAAGAGUUCGAA | UUCGAACUCUUGUUGAGCAGC | | [268-288] ORF |
| 726 | UGAUACUCUUUCCACAAUACU | AGUAUUGUGGAAAGAGUAUCA | | [2092-2112] 3′UTR |
| 727 | GCAUCUCCAACUCUGAGUCUG | CAGACUCAGAGUUGGAGAUGC | Rabbit | [1663-1683] ORF |
| 728 | AGCAUCUCCAACUCUGAGUCU | AGACUCAGAGUUGGAGAUGCU | Rabbit | [1662-1682] ORF |
| 729 | UGAGUAAACAAAGCAUCUCCA | UGGAGAUGCUUUGUUUACUCA | | [1651-1671] ORF |
| 730 | ACCCUGAGUAAACAAAGCAUC | GAUGCUUUGUUUACUCAGGGU | | [1647-1667] ORF |
| 731 | AGGCCUGAAACAAAAGACUUU | AAAGUCUUUUGUUUCAGGCCU | | [1523-1543] ORF |
| 732 | CAACUGCAUGCUGAUUCUCUU | AAGAGAAUCAGCAUGCAGUUG | | [200-220] ORF |
| 733 | GAGCCAGAUGCAGGAAAGGAA | UUCCUUUCCUGCAUCUGGCUC | | [1400-1420] ORF |
| 734 | AAACUACCUAAGAUAGCGGUU | AACCGCUAUCUUAGGUAGUUU | | [1155-1175] ORF |
| 735 | ACCAUCGAGCUACAGAUGAAG | CUUCAUCUGUAGCUCGAUGGU | | [933-953] ORF |
| 736 | UCUGUAUCUCUGUGAGAGGUU | AACCUCUCACAGAGAUACAGA | | [848-868] ORF |
| 737 | GGAUAGUGGGUCCCAUGUUUC | GAAACAUGGGACCCACUAUCC | | [829-849] ORF |
| 738 | GGUUUAUGAUAUUCCACCUAA | UUAGGUGGAAUAUCAUAAACC | | [107-127] ORF |
| 739 | CCAGCUUCAUUUGUUGCUCCG | CGGAGCAACAAAUGAAGCUGG | | [3812-3832] 3′UTR |
| 740 | UGGCCCUUUCUAACCUAUGUG | CACAUAGGUUAGAAAGGGCCA | | [3780-3800] 3′UTR |
| 741 | ACCUCAGUUAUAAGGAGUCUG | CAGACUCCUUAUAACUGAGGU | | [3752-3772] 3′UTR |
| 742 | AGUCAGGCUCUGUUUAUUGUU | AACAAUAAACAGAGCCUGACU | | [3695-3715] 3′UTR |
| 743 | AGAGACACUUAGGUCCAGCCU | AGGCUGGACCUAAGUGUCUCU | | [3610-3630] 3′UTR |
| 744 | CCAGGAUGGUUUCUGCUUAGA | UCUAAGCAGAAACCAUCCUGG | | [3592-3612] 3′UTR |
| 745 | ACCAGGAUGGUUUCUGCUUAG | CUAAGCAGAAACCAUCCUGGU | | [3591-3611] 3′UTR |
| 746 | AACUUCGGUAAUGAGGAAGAA | UUCUUCCUCAUUACCGAAGUU | | [3153-3173] 3′UTR |
| 747 | CUGGGAACUAAAACCAGUUUU | AAAACUGGUUUUAGUUCCCAG | | [3090-3110] 3′UTR |
| 748 | CCCAUUUCUGGGAACUAAAAC | GUUUUAGUUCCCAGAAAUGGG | | [3083-3103] 3′UTR |
| 749 | UCCCAUUUCUGGGAACUAAAA | UUUUAGUUCCCAGAAAUGGGA | | [3082-3102] 3′UTR |
| 750 | UGUGGCCAAGAGCAGGGUUUU | AAAACCCUGCUCUUGGCCACA | | [3041-3061] 3′UTR |
| 751 | AGAGAGUCUCUAAAUCACUGU | ACAGUGAUUUAGAGACUCUCU | | [3017-3037] 3′UTR |
| 752 | AGAGAGAGUCUCUAAAUCACU | AGUGAUUUAGAGACUCUCUCU | | [3015-3035] 3′UTR |
| 753 | GAGAUCAAAUUCACACUCAAU | AUUGAGUGUGAAUUUGAUCUC | | [2971-2991] 3′UTR |
| 754 | CAUCUGUGUGACUAAUGGUUU | AAACCAUUAGUCACACAGAUG | | [2887-2907] 3′UTR |
| 755 | CAAAGUGGUCUUCUCCAGCUA | UAGCUGGAGAAGACCACUUUG | | [2753-2773] 3′UTR |
| 756 | GGAGAAUUAACCUCCUGCCAU | AUGGCAGGAGGUUAAUUCUCC | | [2670-2690] 3′UTR |
| 757 | AGGAUACUAACCAAUAGGAUU | AAUCCUAUUGGUUAGUAUCCU | | [2594-2614] 3′UTR |
| 758 | GGCUAAGGAUACUAACCAAUA | UAUUGGUUAGUAUCCUUAGCC | | [2589-2609] 3′UTR |
| 759 | ACAACUGGACAGGAAUCUCAC | GUGAGAUUCCUGUCCAGUUGU | | [290-310] ORF |
| 760 | AGACAACUGGACAGGAAUCUC | GAGAUUCCUGUCCAGUUGUCU | | [288-308] ORF |
| 761 | CUAUUACUGUCAUAGACCAAA | UUUGGUCUAUGACAGUAAUAG | | [2538-2558] 3′UTR |
| 762 | CGGAUACUCAAGUAAGUUUUG | CAAAACUUACUUGAGUAUCCG | | [2429-2449] 3′UTR |
| 763 | AGAGAGCGGAUACUCAAGUAA | UUACUUGAGUAUCCGCUCUCU | | [2423-2443] 3′UTR |
| 764 | UCAACACUUAUUAGUGACAGU | ACUGUCACUAAUAAGUGUUGA | | [2276-2296] 3′UTR |
| 765 | GUCUCAACACUUAUUAGUGAC | GUCACUAAUAAGUGUUGAGAC | | [2273-2293] 3′UTR |
| 766 | GGUUCAGUAACUUCCCUAAGA | UCUUAGGGAAGUUACUGAACC | | [2029-2049] 3′UTR |
| 767 | GCUCUAUGGUUUUGAGAGCAC | GUGCUCUCAAAACCAUAGAGC | | [1905-1925] 3′UTR |
| 768 | GCUGCCAAAUGCUCAAAUAAU | AUUAUUUGAGCAUUUGGCAGC | | [1763-1783] 3′UTR |
| 769 | AUGUGGGUUGUGCUGCCAAAU | AUUUGGCAGCACAACCCACAU | | [1752-1772] 3′UTR |
| 770 | UGUCUCUUCCAUGAGGAAAUA | UAUUUCCUCAUGGAAGAGACA | | [1730-1750] 3′UTR |
| 771 | ACAAAGCAUCUCCAACUCUGA | UCAGAGUUGGAGAUGCUUUGU | | [1658-1678] ORF |
| 772 | GAAUAGGAGUUUUCCUCUGUG | CACAGAGGAAAACUCCUAUUC | Rabbit | [1606-1626] ORF |
| 773 | GGAGAAAGAUGUGAUCACAGG | CCUGUGAUCACAUCUUUCUCC | Rabbit, Ms | [1505-1525] ORF |
| 774 | UGCACCAUGAUGAGGAGAAAG | CUUUCUCCUCAUCAUGGUGCA | Rabbit, Ms | [1492-1512] ORF |
| 775 | CCAAUCUGAAGCUCAAAAAGA | UCUUUUUGAGCUUCAGAUUGG | | [1309-1329] ORF |
| 776 | CGCCACCAAUCUGAAGCUCAA | UUGAGCUUCAGAUUGGUGGCG | | [1304-1324] ORF |
| 777 | ACGCCACCAAUCUGAAGCUCA | UGAGCUUCAGAUUGGUGGCGU | | [1303-1323] ORF |
| 778 | CAAAUAUUGCAAUAACGCCAC | GUGGCGUUAUUGCAAUAUUUG | | [1289-1309] ORF |
| 779 | UGCCUGAAUUUCAACUGCAUG | CAUGCAGUUGAAAUUCAGGCA | Rat | [189-209] ORF |
| 780 | CCUGCCUGAAUUUCAACUGCA | UGCAGUUGAAAUUCAGGCAGG | Rat | [187-207] ORF |
| 781 | GUGGCUGUGAUAAGCAGGAGU | ACUCCUGCUUAUCACAGCCAC | Rat, Rabbit, Ms | [1120-1140] ORF |
| 782 | GGCUGUUCAAUGCUUGUGGCU | AGCCACAAGCAUUGAACAGCC | Rabbit | [1105-1125] ORF |
| 783 | GAAGACUUCUUUAGUAUCCAU | AUGGAUACUAAAGAAGUCUUC | | [1056-1076] ORF |
| 784 | GGAAGACUUCUUUAGUAUCCA | UGGAUACUAAAGAAGUCUUCC | | [1055-1075] ORF |
| 785 | CCAAUCCCUCAGUUUGCUGGA | UCCAGCAAACUGAGGGAUUGG | | [786-806] ORF |
| 786 | UGGCUGUGCAUAAUAUAACAG | CUGUUAUAUUAUGCACAGCCA | | [721-741] ORF |
| 787 | UCAUCACGCUGUGCCUCAUAU | AUAUGAGGCACAGCGUGAUGA | | [556-576] ORF |
| 788 | GUUAUCUCAAUUUUGCUCGAA | UUCGAGCAAAAUUGAGAUAAC | | [466-486] ORF |
| 789 | UCAGUAGCACUCUCUGAACUU | AAGUUCAGAGAGUGCUACUGA | | [426-446] ORF |
| 790 | UGCUUUUGCAAUUGUGUUUGU | ACAAACACAAUUGCAAAAGCA | | [4132-4152] 3′UTR |
| 791 | AGAGUGUUUCUCCAAGGUUUU | AAAACCUUGGAGAAACACUCU | | [3833-3853] 3′UTR |
| 792 | GGCCCUUUCUAACCUAUGUGC | GCACAUAGGUUAGAAAGGGCC | | [3781-3801] 3′UTR |
| 793 | GGAGUCUGAGAAUAUUGGCCC | GGGCCAAUAUUCUCAGACUCC | | [3765-3785] 3′UTR |
| 794 | UCAGUUAUAAGGAGUCUGAGA | UCUCAGACUCCUUAUAACUGA | | [3755-3775] 3′UTR |
| 795 | AGGGUACCUCAGUUAUAAGGA | UCCUUAUAACUGAGGUACCCU | | [3747-3767] 3′UTR |
| 796 | AGAGGGUACCUCAGUUAUAAG | CUUAUAACUGAGGUACCCUCU | | [3745-3765] 3′UTR |
| 797 | CACCUCAGGUAUCAAUUCAUC | GAUGAAUUGAUACCUGAGGUG | | [3641-3661] 3′UTR |
| 798 | GGUUUCUGCUUAGAGACACUU | AAGUGUCUCUAAGCAGAAACC | | [3599-3619] 3′UTR |
| 799 | GGAUGGUUUCUGCUUAGAGAC | GUCUCUAAGCAGAAACCAUCC | | [3595-3615] 3′UTR |
| 800 | GAAAUGUACCAGGAUGGUUUC | GAAACCAUCCUGGUACAUUUC | | [3584-3604] 3′UTR |
| 801 | UGAAAUGUACCAGGAUGGUUU | AAACCAUCCUGGUACAUUUCA | | [3583-3603] 3′UTR |
| 802 | UGGUCACAAAUUUUGAUGCUG | CAGCAUCAAAAUUUGUGACCA | | [3417-3437] 3′UTR |
| 803 | GGUCUCAUAUUGGUCAUUCCC | GGGAAUGACCAAUAUGAGACC | | [3292-3312] 3′UTR |
| 804 | UUCUGGGAACUAAAACCAGUU | AACUGGUUUUAGUUCCCAGAA | | [3088-3108] 3′UTR |
| 805 | UGCUCCCAUUUCUGGGAACUA | UAGUUCCCAGAAAUGGGAGCA | | [3079-3099] 3′UTR |
| 806 | UGUUCUUAGAACUGCUCCCAU | AUGGGAGCAGUUCUAAGAACA | | [3067-3087] 3′UTR |
| 807 | UUCAAGAGAGAGUCUCUAAAU | AUUUAGAGACUCUCUCUUGAA | | [3011-3031] 3′UTR |
| 808 | CUCUAUACAGAGAUCAAAUUC | GAAUUUGAUCUCUGUAUAGAG | | [2962-2982] 3′UTR |
| 809 | ACCCAGAAACAAAAAUCUCUA | UAGAGAUUUUUGUUUCUGGGU | | [2946-2966] 3′UTR |
| 810 | UGCUCCUUGUGCUAUAAAAUA | UAUUUUAUAGCACAAGGAGCA | | [2711-2731] 3′UTR |
| 811 | CCAUGACUCUGGCUCAUCUAG | CUAGAUGAGCCAGAGUCAUGG | | [2687-2707] 3′UTR |
| 812 | UGGACAGGAAUCUCACCUUUC | GAAAGGUGAGAUUCCUGUCCA | | [295-315] ORF |
| 813 | CACAUUCUUAUUUGAAGCAUG | CAUGCUUCAAAUAAGAAUGUG | | [2630-2650] 3′UTR |
| 814 | CCAAUAGGAUUGCCUGAAGGG | CCCUUCAGGCAAUCCUAUUGG | | [2604-2624] 3′UTR |
| 815 | AAGGAUACUAACCAAUAGGAU | AUCCUAUUGGUUAGUAUCCUU | | [2593-2613] 3′UTR |
| 816 | UCAUAGACCAAAGGAAUCUGA | UCAGAUUCCUUUGGUCUAUGA | | [2547-2567] 3′UTR |
| 817 | UGAAUGAAUUUAGAACCACAC | GUGUGGUUCUAAAUUCAUUCA | | [2459-2479] 3′UTR |
| 818 | CAGAGAGCGGAUACUCAAGUA | UACUUGAGUAUCCGCUCUCUG | | [2422-2442] 3′UTR |
| 819 | GUAAUACUCAUCAUUUACCAC | GUGGUAAAUGAUGAGUAUUAC | | [2392-2412] 3′UTR |
| 820 | GAGAGUGUCUCAACACUUAUU | AAUAAGUGUUGAGACACUCUC | | [2267-2287] 3′UTR |
| 821 | AGGAAACCAAGGAGUAGCUAU | AUAGCUACUCCUUGGUUUCCU | | [2163-2183] 3′UTR |
| 822 | GAGCUGCCUCAGAAUCCUCAA | UUGAGGAUUCUGAGGCAGCUC | | [2113-2133] 3′UTR |
| 823 | CGUGCUGCUCAACAAGAGUUC | GAACUCUUGUUGAGCAGCACG | | [265-285] ORF |
| 824 | GCGUGCUGCUCAACAAGAGUU | AACUCUUGUUGAGCAGCACGC | | [264-284] ORF |
| 825 | UAGCCCAUCUUACUCCAGGUU | AACCUGGAGUAAGAUGGGCUA | | [2071-2091] 3′UTR |
| 826 | UCAGUAACUUCCCUAAGAUUA | UAAUCUUAGGGAAGUUACUGA | | [2032-2052] 3′UTR |
| 827 | CCUUAAAGGAAUGUCAAAGAU | AUCUUUGACAUUCCUUUAAGG | | [1852-1872] 3′UTR |
| 828 | CCCUUAAAGGAAUGUCAAAGA | UCUUUGACAUUCCUUUAAGGG | | [1851-1871] 3′UTR |
| 829 | UGGUUUUCCCUUAAAGGAAUG | CAUUCCUUUAAGGGAAAACCA | | [1844-1864] 3′UTR |
| 830 | CUAUAAUGUAAUGGUUUUCCC | GGGAAAACCAUUACAUUAUAG | | [1833-1853] 3′UTR |
| 831 | GGAGUUUUCCUCUGUGGACCU | AGGUCCACAGAGGAAAACUCC | Rabbit | [1611-1631] ORF |
| 832 | CCUAAUACCAGAAUAGGAGUU | AACUCCUAUUCUGGUAUUAGG | Rabbit | [1596-1616] ORF |
| 833 | GCCUGAAACAAAAGACUUUGU | ACAAAGUCUUUUGUUUCAGGC | | [1525-1545] ORF |
| 834 | GGCCUGAAACAAAAGACUUUG | CAAAGUCUUUUGUUUCAGGCC | | [1524-1544] ORF |
| 835 | CUGAAUUUCAACUGCAUGCUG | CAGCAUGCAGUUGAAAUUCAG | Rat | [192-212] ORF |
| 836 | ACCAAUCUGAAGCUCAAAAAG | CUUUUUGAGCUUCAGAUUGGU | | [1308-1328] ORF |
| 837 | AUAGCGGUUGAUGGGCCCUUU | AAAGGGCCCAUCAACCGCUAU | | [1167-1187] ORF |
| 838 | CAUUUUUGUCAAGUGCCCAAA | UUUGGGCACUUGACAAAAAUG | | [986-1006] ORF |
| 839 | GUGGGACAAUACAUUUUUGUC | GACAAAAAUGUAUUGUCCCAC | | [975-995] ORF |
| 840 | GAAAGAGAAUAAAGAACCCUG | CAGGGUUCUUUAUUCUCUUUC | | [484-504] ORF |
| 841 | AGGCAAAAUGAAAGUUAUCUC | GAGAUAACUUUCAUUUUGCCU | | [453-473] ORF |
| 842 | CCGAGUCAAUAAUUCUGAUCC | GGAUCAGAAUUAUUGACUCGG | | [401-421] ORF |
| 843 | GAAUGGUGUGUGAAUGCCCGA | UCGGGCAUUCACACACCAUUC | | [384-404] ORF |
| 844 | CUGCUUUUGCAAUUGUGUUUG | CAAACACAAUUGCAAAAGCAG | | [4131-4151] 3′UTR |
| 845 | UGAGAAUAUUGGCCCUUUCUA | UAGAAAGGGCCAAUAUUCUCA | | [3771-3791] 3′UTR |
| 846 | CUCAGUUAUAAGGAGUCUGAG | CUCAGACUCCUUAUAACUGAG | | [3754-3774] 3′UTR |
| 847 | GUCAGGCUCUGUUUAUUGUUU | AAACAAUAAACAGAGCCUGAC | | [3696-3716] 3′UTR |
| 848 | ACUGCACCUCAGGUAUCAAUU | AAUUGAUACCUGAGGUGCAGU | | [3637-3657] 3′UTR |
| 849 | CUGCUUAGAGACACUUAGGUC | GACCUAAGUGUCUCUAAGCAG | | [3604-3624] 3′UTR |
| 850 | UGGUUUCUGCUUAGAGACACU | AGUGUCUCUAAGCAGAAACCA | | [3598-3618] 3′UTR |
| 851 | UGCACUUCACUCUGCGAUUCA | UGAAUCGCAGAGUGAAGUGCA | | [338-358] ORF |
| 852 | UCACAAAUUUUGAUGCUGAGG | CCUCAGCAUCAAAAUUUGUGA | | [3420-3440] 3′UTR |
| 853 | GGCAGAAGACUGGUUUAGGAG | CUCCUAAACCAGUCUUCUGCC | | [3194-3214] 3′UTR |
| 854 | GCUCCCAUUUCUGGGAACUAA | UUAGUUCCCAGAAAUGGGAGC | | [3080-3100] 3′UTR |
| 855 | UCACACUCAAUAGUAUGUUCU | AGAACAUACUAUUGAGUGUGA | | [2981-3001] 3′UTR |
| 856 | UCCUAAAAUUAACAACCCAGA | UCUGGGUUGUUAAUUUUAGGA | | [2932-2952] 3′UTR |
| 857 | CACAAAAUGGAUCGCAUCUGU | ACAGAUGCGAUCCAUUUUGUG | | [2873-2893] 3′UTR |
| 858 | ACCUUUCAUAAAAUGGUGGCA | UGCCACCAUUUUAUGAAAGGU | | [309-329] ORF |
| 859 | ACACACAAAAUGGAUCGCAUC | GAUGCGAUCCAUUUUGUGUGU | | [2870-2890] 3′UTR |
| 860 | CACACACAAAAUGGAUCGCAU | AUGCGAUCCAUUUUGUGUGUG | | [2869-2889] 3′UTR |
| 861 | CAUAGACCAAAGGAAUCUGAU | AUCAGAUUCCUUUGGUCUAUG | | [2548-2568] 3′UTR |
| 862 | GAUGUUCUUUCUACAGAAGAA | UUCUUCUGUAGAAAGAACAUC | | [2349-2389] 3′UTR |
| 863 | CAACAAGAGUUCGAAGACAAC | GUUGUCUUCGAACUCUUGUUG | | [274-294] ORF |
| 864 | GCCCAUCUUACUCCAGGUUUG | CAAACCUGGAGUAAGAUGGGC | | [2073-2093] 3′UTR |
| 865 | AAAGGUUCAGUAACUUCCCUA | UAGGGAAGUUACUGAACCUUU | | [2026-2046] 3′UTR |
| 866 | CUCUAUGGUUUUGAGAGCACU | AGUGCUCUCAAAACCAUAGAG | | [1906-1926] 3′UTR |
| 867 | AGCUCUAUGGUUUUGAGAGCA | UGCUCUCAAAACCAUAGAGCU | | [1904-1924] 3′UTR |
| 868 | GGUUUUCCCUUAAAGGAAUGU | ACAUUCCUUUAAGGGAAAACC | | [1845-1865] 3′UTR |
| 869 | UGCUGCCAAAUGCUCAAAUAA | UUAUUUGAGCAUUUGGCAGCA | | [1762-1782] 3′UTR |
| 870 | UGUGCUGCCAAAUGCUCAAAU | AUUUGAGCAUUUGGCAGCACA | | [1760-1780] 3′UTR |
| 871 | GCUGAUUCUCUUGCCAGUCUG | CAGACUGGCAAGAGAAUCAGC | | [209-229] ORF |
| 872 | CAGGCCAAUCACUUUGCUGUG | CACAGCAAAGUGAUUGGCCUG | Rat, Rabbit, Ms | [1473-1493] ORF |
| 873 | CACACAUGCCUUUGAGUGGUU | AACCACUCAAAGGCAUGUGUG | Rabbit | [1355-1375] ORF |
| 874 | GCCUGCCUGAAUUUCAACUGC | GCAGUUGAAAUUCAGGCAGGC | Rat | [186-206] ORF |
| 875 | UGCAGCCUGCCUGAAUUUCAA | UUGAAAUUCAGGCAGGCUGCA | Rat, Ms | [182-202] ORF |
| 876 | CGUGGAAACUACCUAAGAUAG | CUAUCUUAGGUAGUUUCCACG | | [1150-1170] ORF |
| 877 | UCACCCUUUCAAAACCAUCGA | UCGAUGGUUUUGAAAGGGUGA | | [920-940] ORF |
| 878 | CAAGGUGGUCACUCACCCUUU | AAAGGGUGAGUGACCACCUUG | Rabbit | [908-928] ORF |
| 879 | GGAAAUGGAUAGUGGGUCCCA | UGGGACCCACUAUCCAUUUCC | Rabbit | [823-843] ORF |
| 880 | CCCUCCUAUGACUUGGAAAUG | CAUUUCCAAGUCAUAGGAGGG | Rat, Ms | [809-829] ORF |
| 881 | ACACACAUCAUCUCUUUGUGA | UCACAAAGAGAUGAUGUGUGU | | [634-654] ORF |
| 882 | GCUCCGAGAGUGUUUCUCCAA | UUGGAGAAACACUCUCGGAGC | | [3827-3847] 3′UTR |
| 883 | GCGAUUCACACCAUUGCACAU | AUGUGCAAUGGUGUGAAUCGC | | [351-371] ORF |
| 884 | GCUUAGAGACACUUAGGUCCA | UGGACCUAAGUGUCUCUAAGC | | [3606-3626] 3′UTR |
| 885 | GUACCAGGAUGGUUUCUGCUU | AAGCAGAAACCAUCCUGGUAC | | [3589-3609] 3′UTR |
| 886 | AGAACUCAGUUGUUCUUUGUC | GACAAAGAACAACUGAGUUCU | | [3474-3494] 3′UTR |
| 887 | CACAUACUGAUGUUGUUUUGA | UCAAAACAACAUCAGUAUGUG | | [3367-3387] 3′UTR |
| 888 | ACAACCCAGAAACAAAAAUCU | AGAUUUUUGUUUCUGGGUUGU | | [2943-2963] 3′UTR |
| 889 | ACAAAAUGGAUCGCAUCUGUG | CACAGAUGCGAUCCAUUUUGU | | [2874-2894] 3′UTR |
| 890 | UGAAGGGUUCUGCACAUUCUU | AAGAAUGUGCAGAACCCUUCA | | [2618-2638] 3′UTR |
| 891 | UCUAUUACUGUCAUAGACCAA | UUGGUCUAUGACAGUAAUAGA | | [2537-2557] 3′UTR |
| 892 | GGAUACUCAAGUAAGUUUUGU | ACAAAACUUACUUGAGUAUCC | | [2430-2450] 3′UTR |
| 893 | CUGGGCCAGAUGUUCUUUCUA | UAGAAAGAACAUCUGGCCCAG | | [2341-2361] 3′UTR |
| 894 | ACAUGUUGAGAGUGUCUCAAC | GUUGAGACACUCUCAACAUGU | | [2260-2280] 3′UTR |
| 895 | CUGCUCAACAAGAGUUCGAAG | CUUCGAACUCUUGUUGAGCAG | | [269-289] ORF |
| 896 | AACCAAGGAGUAGCUAUAUAU | AUAUAUAGCUACUCCUUGGUU | | [2167-2187] 3′UTR |
| 897 | CCCAAAAGAAGAAGGAAACCA | UGGUUUCCUUCUUCUUUUGGG | | [2151-2171] 3′UTR |
| 898 | UUCAGUAACUUCCCUAAGAUU | AAUCUUAGGGAAGUUACUGAA | | [2031-2051] 3′UTR |
| 899 | AGGUUCAGUAACUUCCCUAAG | CUUAGGGAAGUUACUGAACCU | | [2028-2048] 3′UTR |
| 900 | AAGGUUCAGUAACUUCCCUAA | UUAGGGAAGUUACUGAACCUU | | [2027-2047] 3′UTR |
| 901 | CCUCUCAGUAAUGUCAGUGGA | UCCACUGACAUUACUGAGAGG | | [1952-1972] 3′UTR |
| 902 | GGAGCUCUAUGGUUUUGAGAG | CUCUCAAAACCAUAGAGCUCC | | [1902-1922] 3′UTR |
| 903 | CUGCCAAAUGCUCAAAUAAUG | CAUUAUUUGAGCAUUUGGCAG | | [1764-1784] 3′UTR |
| 904 | GGAAAACUUCUAACUUGUCUC | GAGACAAGUUAGAAGUUUUCC | | [1715-1735] ORF + 3′UTR |
| 905 | CUGAUUCUCUUGCCAGUCUGU | ACAGACUGGCAAGAGAAUCAG | | [210-230] ORF |
| 906 | GCACCAUGAUGAGGAGAAAGA | UCUUUCUCCUCAUCAUGGUGC | Rabbit, Ms | [1493-1513] ORF |
| 907 | ACACAUGCCUUUGAGUGGUUU | AAACCACUCAAAGGCAUGUGU | Rabbit | [1356-1376] ORF |
| 908 | GGACACACAUGCCUUUGAGUG | CACUCAAAGGCAUGUGUGUCC | | [1352-1372] ORF |
| 909 | GCAAUAACGCCACCAAUCUGA | UCAGAUUGGUGGCGUUAUUGC | | [1297-1317] ORF |
| 910 | CCAGUGAAGAUGUGUUCAGCU | AGCUGAACACAUCUUCACUGG | Rat, Ms | [1195-1215] ORF |
| 911 | ACCUAAGUUCUUUUACACAAG | CUUGUGUAAAAGAACUUAGGU | | [122-142] ORF |
| 912 | CACCCUUUCAAAACCAUCGAG | CUCGAUGGUUUUGAAAGGGUG | | [921-941] ORF |
| 913 | UCCUAUGACUUGGAAAUGGAU | AUCCAUUUCCAAGUCAUAGGA | Rat, Ms | [812-832] ORF |
| 914 | GAAACCCUCCUAUGACUUGGA | UCCAAGUCAUAGGAGGGUUUC | Rat, Ms | [805-825] ORF |
| 915 | UCAUUGGCCUUGCCAUCCAUG | CAUGGAUGGCAAGGCCAAUGA | | [661-681] ORF |
| 916 | ACAUCAUCUCUUUGUGAUCUU | AAGAUCACAAAGAGAUGAUGU | | [638-658] ORF |
| 917 | CUGGUAUUACCGGGUUUAUGA | UCAUAAACCCGGUAAUACCAG | | [95-115] ORF |
| 918 | CCAAAGUAUGAACUGGAGAGG | CCUCUCCAGUUCAUACUUUGG | | [3729-3749] 3′UTR |
| 919 | ACACCAAAGUAUGAACUGGAG | CUCCAGUUCAUACUUUGGUGU | | [3726-3746] 3′UTR |
| 920 | UCAGGCUCUGUUUAUUGUUUC | GAAACAAUAAACAGAGCCUGA | | [3697-3717] 3′UTR |
| 921 | UGAGUCAGGCUCUGUUUAUUG | CAAUAAACAGAGCCUGACUCA | | [3693-3713] 3′UTR |
| 922 | GCCUUUCUGAAAUGUACCAGG | CCUGGUACAUUUCAGAAAGGC | | [3576-3596] 3′UTR |
| 923 | AGCCUUUCUGAAAUGUACCAG | CUGGUACAUUUCAGAAAGGCU | | [3575-3595] 3′UTR |
| 924 | UCAUACUACUAUUCCUUUCGU | ACGAAAGGAAUAGUAGUAUGA | | [3493-3513] 3′UTR |
| 925 | CUGGUCACAAAUUUUGAUGCU | AGCAUCAAAAUUUGUGACCAG | | [3416-3436] 3′UTR |
| 926 | UGAAGGAGGUAUACACAUACU | AGUAUGUGUAUACCUCCUUCA | | [3354-3374] 3′UTR |
| 927 | CAACUUCGGUAAUGAGGAAGA | UCUUCCUCAUUACCGAAGUUG | | [3152-3172] 3′UTR |
| 928 | GGUGGCAUGGAUGAUUGCACU | AGUGCAAUCAUCCAUGCCACC | | [323-343] ORF |
| 929 | UGUUUAGAACUCUUCAACUUC | GAAGUUGAAGAGUUCUAAACA | | [3138-3158] 3′UTR |
| 930 | UCACUGUUAGUGUGGCCAAGA | UCUUGGCCACACUAACAGUGA | | [3031-3051] 3′UTR |
| 931 | AGUCUCUAAAUCACUGUUAGU | ACUAACAGUGAUUUAGAGACU | | [3021-3041] 3′UTR |
| 932 | CACACUCAAUAGUAUGUUCUG | CAGAACAUACUAUUGAGUGUG | | [2982-3002] 3′UTR |
| 933 | CAGAGAUCAAAUUCACACUCA | UGAGUGUGAAUUUGAUCUCUG | | [2969-2989] 3′UTR |
| 934 | ACAGAGAUCAAAUUCACACUC | GAGUGUGAAUUUGAUCUCUGU | | [2968-2988] 3′UTR |
| 935 | CAACCCAGAAACAAAAAUCUC | GAGAUUUUUGUUUCUGGGUUG | | [2944-2964] 3′UTR |
| 936 | UCCGUAAAUACACACACAAAA | UUUUGUGUGUGUAUUUACGGA | | [2859-2879] 3′UTR |
| 937 | CCAAAGUGGUCUUCUCCAGCU | AGCUGGAGAAGACCACUUUGG | | [2752-2772] 3′UTR |
| 938 | GAGGUGGAGAAUUAACCUCCU | AGGAGGUUAAUUCUCCACCUC | | [2665-2685] 3′UTR |
| 939 | UGCACAUUCUUAUUUGAAGCA | UGCUUCAAAUAAGAAUGUGCA | | [2628-2648] 3′UTR |
| 940 | AGGCUAAGGAUACUAACCAAU | AUUGGUUAGUAUCCUUAGCCU | | [2588-2608] 3′UTR |
| 941 | CAAAGGAAUCUGAUUCUCCCU | AGGGAGAAUCAGAUUCCUUUG | | [2555-2575] 3′UTR |
| 942 | AGACCAAAGGAAUCUGAUUCU | AGAAUCAGAUUCCUUUGGUCU | | [2551-2571] 3′UTR |
| 943 | GCAGAGAGCGGAUACUCAAGU | ACUUGAGUAUCCGCUCUCUGC | | [2421-2441] 3′UTR |
| 944 | ACAGUAUUGACAUCUGAGCAU | AUGCUCAGAUGUCAAUACUGU | | [2292-2312] 3′UTR |
| 945 | CACUUAUUAGUGACAGUAUUG | CAAUACUGUCACUAAUAAGUG | | [2280-2300] 3′UTR |
| 946 | CUCAACACUUAUUAGUGACAG | CUGUCACUAAUAAGUGUUGAG | | [2275-2295] 3′UTR |
| 947 | CCACAAUACUGAGCUGCCUCA | UGAGGCAGCUCAGUAUUGUGG | | [2103-2123] 3′UTR |
| 948 | UGGAGCUCUAUGGUUUUGAGA | UCUCAAAACCAUAGAGCUCCA | | [1901-1921] 3′UTR |
| 949 | CUAACUUGUCUCUUCCAUGAG | CUCAUGGAAGAGACAAGUUAG | | [1724-1744] ORF + 3′UTR |
| 950 | CAAGGAAAACUUCUAACUUGU | ACAAGUUAGAAGUUUUCCUUG | | [1712-1732] ORF + 3′UTR |
| 951 | ACAGGCCUGAAACAAAAGACU | AGUCUUUUGUUUCAGGCCUGU | | [1521-1541] ORF |
| 952 | AGCCAGAUGCAGGAAAGGAAC | GUUCCUUUCCUGCAUCUGGCU | | [1401-1421] ORF |
| 953 | CAAUCUGAAGCUCAAAAAGAU | AUCUUUUUGAGCUUCAGAUUG | | [1310-1330] ORF |
| 954 | CCAUUCUCAAGUCAGUCUGGU | ACCAGACUGACUUGAGAAUGG | | [1267-1287] ORF |
| 955 | GCCAGUGAAGAUGUGUUCAGC | GCUGAACACAUCUUCACUGGC | Rat, Ms | [1194-1214] ORF |
| 956 | GCACUGCCAGUGAAGAUGUGU | ACACAUCUUCACUGGCAGUGC | | [1189-1209] ORF |
| 957 | UGCUUGUGGCUGUGAUAAGCA | UGCUUAUCACAGCCACAAGCA | Rabbit, Ms | [1115-1135] ORF |
| 958 | GGAGUGGCACCCUUUUACACU | AGUGUAAAAGGGUGCCACUCC | | [1019-1039] ORF |
| 959 | CCUAAGUUCUUUUACACAAGA | UCUUGUGUAAAAGAACUUAGG | | [123-143] ORF |
| 960 | CUCCUAUGACUUGGAAAUGGA | UCCAUUUCCAAGUCAUAGGAG | Rat, Ms | [811-831] ORF |
| 961 | AAACCCUCCUAUGACUUGGAA | UUCCAAGUCAUAGGAGGGUUU | Rat, Ms | [806-826] ORF |
| 962 | AUCUCAAUUUUGCUCGAAAGA | UCUUUCGAGCAAAAUUGAGAU | | [469-489] ORF |
| 963 | GUGUUAUCCAGUUAUGAGAUA | UAUCUCAUAACUGGAUAACAC | | [3900-3920] 3′UTR |
| 964 | CAGGAUGGUUUCUGCUUAGAG | CUCUAAGCAGAAACCAUCCUG | | [3593-3613] 3′UTR |
| 965 | AAGAACUCAGUUGUUCUUUGU | ACAAAGAACAACUGAGUUCUU | | [3473-3493] 3′UTR |
| 966 | UCAACUUCGGUAAUGAGGAAG | CUUCCUCAUUACCGAAGUUGA | | [3151-3171] 3′UTR |
| 967 | UCUGGGAACUAAAACCAGUUU | AAACUGGUUUUAGUUCCCAGA | | [3089-3109] 3′UTR |
| 968 | CUGCUCCCAUUUCUGGGAACU | AGUUCCCAGAAAUGGGAGCAG | | [3078-3098] 3′UTR |
| 969 | UUCCGUAAAUACACACACAAA | UUUGUGUGUGUAUUUACGGAA | | [2858-2878] 3′UTR |
| 970 | GGAGGUGGAGAAUUAACCUCC | GGAGGUUAAUUCUGCACCUCC | | [2664-2684] 3′UTR |
| 971 | CUGCACAUUCUUAUUUGAAGC | GCUUCAAAUAAGAAUGUGCAG | | [2627-2647] 3′UTR |
| 972 | GGAAUCUGAUUCUCCCUAGGG | CCCUAGGGAGAAUCAGAUUCC | | [2559-2579] 3′UTR |
| 973 | UGGCAGAGAGCGGAUACUCAA | UUGAGUAUCCGCUCUCUGCCA | | [2419-2439] 3′UTR |
| 974 | CCAGAUGUUCUUUCUACAGAA | UUCUGUAGAAAGAACAUCUGG | | [2346-2366] 3′UTR |
| 975 | GCCAGAUGUUCUUUCUACAGA | UCUGUAGAAAGAACAUCUGGC | | [2345-2365] 3′UTR |
| 976 | AGUGACAGUAUUGACAUCUGA | UCAGAUGUCAAUACUGUCACU | | [2288-2308] 3′UTR |
| 977 | GUUGAGAGUGUCUCAACACUU | AAGUGUUGAGACACUCUCAAC | | [2264-2284] 3′UTR |
| 978 | GCUCAACAAGAGUUCGAAGAC | GUCUUCGAACUCUUGUUGAGC | | [271-291] ORF |
| 979 | GAAGGAAACCAAGGAGUAGCU | AGCUACUCCUUGGUUUCCUUC | | [2161-2181] 3′UTR |
| 980 | UCCACAAUACUGAGCUGCCUC | GAGGCAGCUCAGUAUUGUGGA | | [2102-2122] 3′UTR |
| 981 | CUAGCCCAUCUUACUCCAGGU | ACCUGGAGUAAGAUGGGCUAG | | [2070-2090] 3′UTR |
| 982 | GGAAUGUCAAAGAUUGUUUGA | UCAAACAAUCUUUGACAUUCC | | [1859-1879] 3′UTR |
| 983 | ACUUGUCUCUUCCAUGAGGAA | UUCCUCAUGGAAGAGACAAGU | | [1727-1747] 3′UTR |
| 984 | AGGAGAAAGAUGUGAUCACAG | CUGUGAUCACAUCUUUCUCCU | Rabbit, Ms | [1504-1524] ORF |
| 985 | UGGGAUGAGUCUCAGGCCAAU | AUUGGCCUGAGACUCAUCCCA | | [1461-1481] ORF |
| 986 | GUACAAAUAUUGCAAUAACGC | GCGUUAUUGCAAUAUUUGUAC | | [1286-1306] ORF |
| 987 | AGUGAAGAUGUGUUCAGCUAU | AUAGCUGAACACAUCUUCACU | Rat, Rabbit, Ms | [1197-1217] ORF |
| 988 | UGGUCACUCACCCUUUCAAAA | UUUUGAAAGGGUGAGUGACCA | Rabbit | [913-933] ORF |
| 989 | AGGUGGUCACUCACCCUUUCA | UGAAAGGGUGAGUGACCACCU | Rabbit | [910-930] ORF |
| 990 | ACCCUCCUAUGACUUGGAAAU | AUUUCCAAGUCAUAGGAGGGU | Rat, Ms | [808-828] ORF |
| 991 | AACCCUCCUAUGACUUGGAAA | UUUCCAAGUCAUAGGAGGGUU | Rat, Ms | [807-827] ORF |
| 992 | CAGGCAUCACUGGAGUUGUCA | UGACAACUCCAGUGAUGCCUG | | [538-558] ORF |
| 993 | CUCAAUUUUGCUCGAAAGAGA | UCUCUUUCGAGCAAAAUUGAG | | [471-491] ORF |
| 994 | GUCAUACUACUAUUCCUUUCG | CGAAAGGAAUAGUAGUAUGAC | | [3492-3512] 3′UTR |
| 995 | GGGCAGAAGACUGGUUUAGGA | UCCUAAACCAGUCUUCUGCCC | | [3193-3213] 3′UTR |
| 996 | CUAAAACCAGUUUUAUUUGCC | GGCAAAUAAAACUGGUUUUAG | | [3097-3117] 3′UTR |
| 997 | GAUCAAAUUCACACUCAAUAG | CUAUUGAGUGUGAAUUUGAUC | | [2973-2993] 3′UTR |
| 998 | ACACACACAAAAUGGAUCGCA | UGCGAUCCAUUUUGUGUGUGU | | [2868-2888] 3′UTR |
| 999 | UGGAGAAUUAACCUCCUGCCA | UGGCAGGAGGUUAAUUCUCCA | | [2669-2689] 3′UTR |
| 1000 | UGCCUGAAGGGUUCUGCACAU | AUGUGCAGAACCCUUCAGGCA | | [2614-2634] 3′UTR |

TABLE G
19-mer oligonucleotides to NADPH oxidase 5 (NOX5)

| | | | | |
|---|---|---|---|---|
| Number | Sense siRNA | AntiSense siRNA | Other Sp | Human-20127623 ORF: 302-2545 |

| | | | |
|---|---|---|---|
| 1 | CCCUAUUUGACUCCGAUAG | CUAUCGGAGUCAAAUAGGG | [447-465] ORF |
| 2 | CUAUUUGACUCCGAUAGAA | UUCUAUCGGAGUCAAAUAG | [449-467] ORF |
| 3 | AGAUACUUUUGGAGAAACA | UGUUUCUCCAAAAGUAUCU | [1902-1920] ORF |
| 4 | CUGAGAUACUUUUGGAGAA | UUCUCCAAAAGUAUCUCAG | [1899-1917] ORF |
| 5 | CCCUUUUUUUCACUAUAGA | UCUAUAGUGAAAAAAAGGG | [1645-1663] ORF |
| 6 | GUGCCUGGAAUCUUGUUUU | AAAACAAGAUUCCAGGCAC | [1520-1538] ORF |
| 7 | GAACUUUGUACUCCAGGCU | AGCCUGGAGUACAAAGUUC | [1249-1267] ORF |
| 8 | GAGAUACUUUUGGAGAAAC | GUUUCUCCAAAAGUAUCUC | [1901-1919] ORF |
| 9 | GGCUGUAUGAGUCCUUCAA | UUGAAGGACUCAUACAGCC | [1797-1815] ORF |
| 10 | AGGGCUCUGAGAUACUUUU | AAAAGUAUCUCAGAGCCCU | [1893-1911] ORF |
| 11 | GAAUCUUGUUUUUCCUGGA | UCCAGGAAAAACAAGAUUC | [1527-1545] ORF |
| 12 | GGUGCCUGGAAUCUUGUUU | AAACAAGAUUCCAGGCACC | [1519-1537] ORF |
| 13 | ACACUGUGAACUUUGUACU | AGUACAAAGUUCACAGUGU | [1242-1260] ORF |
| 14 | UGCUGACUAAACUGGAGAU | AUCUCCAGUUUAGUCAGCA | [2202-2220] ORF |
| 15 | CCUAUUUGACUCCGAUAGA | UCUAUCGGAGUCAAAUAGG | [448-466] ORF |
| 16 | GCAGUUUAAGACCAUUGCA | UGCAAUGGUCUUAAACUGC | [346-364] ORF |
| 17 | GCUGACUAAACUGGAGAUG | CAUCUCCAGUUUAGUCAGC | [2203-2221] ORF |
| 18 | AGAAAAGAAAGCAUACUUG | CAAGUAUGCUUUCUUUUCU | [2070-2088] ORF |
| 19 | UACUUUUGGAGAAACACAA | UUGUGUUUCUCCAAAAGUA | [1905-1923] ORF |
| 20 | CCAAGAGAAUUUCUAGCCU | AGGCUAGAAAUUCUCUUGG | [2530-2548] ORF + 3′UTR |
| 21 | CCAUUCUGCAGAGUAUCAU | AUGAUACUCUGCAGAAUGG | [2040-2058] ORF |
| 22 | GGGCUCUGAGAUACUUUUG | CAAAAGUAUCUCAGAGCCC | [1894-1912] ORF |
| 23 | GGACAAACAGGCUGUAUGA | UCAUACAGCCUGUUUGUCC | [1788-1806] ORF |
| 24 | CCCAUGGACAAACUCAAAU | AUUUGAGUUUGUCCAUGGG | [527-545] ORF |
| 25 | UGCAUGUGAAAGAGUCCUU | AAGGACUCUUUCACAUGCA | [408-426] ORF |
| 26 | CAGCAGUUUAAGACCAUUG | CAAUGGUCUUAAACUGCUG | [344-362] ORF |
| 27 | UCAUGUUGUAUCUGGAAUU | AAUUCCAGAUACAACAUGA | [228-246] 5′UTR |
| 28 | ACAGGCACCAGAAAAGAAA | UUUCUUUUCUGGUGCCUGU | [2061-2079] ORF |
| 29 | GGCUCUGAGAUACUUUUGG | CCAAAAGUAUCUCAGAGCC | [1895-1913] ORF |
| 30 | CCAAGGGCUCUGAGAUACU | AGUAUCUCAGAGCCCUUGG | [1890-1908] ORF |
| 31 | UCAGCCUGCAAGAAUUCAA | UUGAAUUCUUGCAGGCUGA | [381-399] ORF |
| 32 | UGGACUUUAUCUGGAUCAA | UUGAUCCAGAUAAAGUCCA | [2148-2166] ORF |
| 33 | AGGUCACUCAUCUCCUCAU | AUGAGGAGAUGAGUGACCU | [1617-1835] ORF |
| 34 | CCGUGUGCAUCAUGGAAGU | ACUUCCAUGAUGCACACGG | [1581-1599] ORF |
| 35 | CAGCCUGCAAGAAUUCAAA | UUUGAAUUCUUGCAGGCUG | [382-400] ORF |
| 36 | GGGCCAUUGUGAGAAGUUC | GAACUUCUCACAAUGGCCC | [2497-2515] ORF |
| 37 | AGCAAGGUGUUCCAGAAAG | CUUUCUGGAACACCUUGCU | [2408-2426] ORF |
| 38 | CUGCUGACUAAACUGGAGA | UCUCCAGUUUAGUCAGCAG | [2201-2219] ORF |
| 39 | CCUGCUGACUAAACUGGAG | CUCCAGUUUAGUCAGCAGG | [2200-2218] ORF |
| 40 | GGACUUUAUCUGGAUCAAC | GUUGAUCCAGAUAAAGUCC | [2149-2167] ORF |
| 41 | AGGUGGACUUUAUCUGGAU | AUCCAGAUAAAGUCCACCU | [2145-2183] ORF |
| 42 | GUCAUGUUGUAUCUGGAAU | AUUCCAGAUACAACAUGAC | [227-245] 5′UTR |
| 43 | CUCUGAGAUACUUUUGGAG | CUCCAAAAGUAUCUCAGAG | [1897-1915] ORF |
| 44 | AGAGUCAAAGGUCGUCCAA | UUGGACGACCUUUGACUCU | [1875-1893] ORF |
| 45 | UGCCUGGAAUCUUGUUUUU | AAAAACAAGAUUCCAGGCA | [1521-1539] ORF |
| 46 | UACCACUGGACCAGAACAU | AUGUUCUGGUCCAGUGGUA | [1161-1179] ORF |
| 47 | ACAAACUCAAAUUCCUCUU | AAGAGGAAUUUGAGUUUGU | [534-552] ORF |
| 48 | UGGGCAAGAAUGACAUGAA | UUCAUGUCAUUCUUGCCCA | [2286-2304] ORF |
| 49 | GCAGUCAUGUUGUAUCUGG | CCAGAUACAACAUGACUGC | [224-242] 5′UTR |
| 50 | GACAAUGAGAAAGAGUCAA | UUGACUCUUUCUCAUUGUC | [1864-1882] ORF |
| 51 | GCUGUAUGAGUCCUUCAAG | CUUGAAGGACUCAUACAGC | [1798-1816] ORF |
| 52 | UGGACAAACAGGCUGUAUG | CAUACAGCCUGUUUGUCCA | [1787-1805] ORF |
| 53 | CGAGGUCUCAAUCCAUUGC | GCAAUGGAUUGAGACCUCG | [191-209] 5′UTR |
| 54 | CCUUUUUUUCACUAUAGAC | GUCUAUAGUGAAAAAAAGG | [1646-1654] ORF |
| 55 | UGUGCAUCAUGGAAGUCAA | UUGACUUCCAUGAUGCACA | [1584-1602] ORF |
| 56 | UCAUCUUUCAUGGGCCCAA | UUGGGCCCAUGAAAGAUGA | [1482-1500] ORF |
| 57 | UGCUCCUCCUCAUGUUCAU | AUGAACAUGAGGAGGAGCA | [1377-1395] ORF |
| 58 | GCAAGAAUUCAAAGCAGCU | AGCUGCUUUGAAUUCUUGC | [388-406] ORF |
| 59 | AGCGGUCUUUCGAGUGGUU | AACCACUCGAAAGACCGCU | [2175-2193] ORF |
| 60 | CCAGAAAAGAAAGCAUACU | AGUAUGCUUUCUUUUCUGG | [2068-2086] ORF |
| 61 | CAAUGAGAAAGAGUCAAAG | CUUUGACUCUUUCUCAUUG | [1866-1884] ORF |
| 62 | CUGGUAUGCAGUCAUGUUG | CAACAUGACUGCAUACCAG | [217-235] 5′UTR |
| 63 | AGGAGUGUGACAAUGAGAA | UUCUCAUUGUCACACUCCU | [1856-1874] ORF |
| 64 | GUGGACAAACAGGCUGUAU | AUACAGCCUGUUUGUCCAC | [1786-1804] ORF |
| 65 | CCAAGGUCACUCAUCUCCU | AGGAGAUGAGUGACCUUGG | [1614-1632] ORF |
| 66 | UGCUGCUCCUCCUCAUGUU | AACAUGAGGAGGAGCAGCA | [1374-1392] ORF |
| 67 | CAAGAAUUCAAAGCAGCUC | GAGCUGCUUUGAAUUCUUG | [389-407] ORF |
| 68 | AGGGCCAUUGUGAGAAGUU | AACUUCUCACAAUGGCCCU | [2496-2514] ORF |
| 69 | UGAAGGGCCAUUGUGAGAA | UUCUCACAAUGGCCCUUCA | [2493-2511] ORF |
| 70 | GUGGACUUUAUCUGGAUCA | UGAUCCAGAUAAAGUCCAC | [2147-2165] ORF |
| 71 | UCCAAGACAACAUGAAGCU | AGCUUCAUGUUGUCUUGGA | [2121-2139] ORF |
| 72 | CAGAAAAGAAAGCAUACUU | AAGUAUGCUUUCUUUUCUG | [2069-2087] ORF |
| 73 | GCACCAGAAAAGAAAGCAU | AUGCUUUCUUUUCUGGUGC | [2065-2083] ORF |
| 74 | GCUUCCAUUCUGCAGAGUA | UACUCUGCAGAAUGGAAGC | [2036-2054] ORF |
| 75 | GCUACAUCGAUGGGCCUUA | UAAGGCCCAUCGAUGUAGC | [1941-1959] ORF |
| 76 | CAAACAGGCUGUAUGAGUC | GACUCAUACAGCCUGUUUG | [1791-1809] ORF |
| 77 | ACACUAUCUGGCUGCACAU | AUGUGCAGCCAGAUAGUGU | [1752-1770] ORF |
| 78 | CUGAGCAGAAAGACACUAU | AUAGUGUCUUUCUGCUCAG | [1740-1758] ORF |
| 79 | CCUGAGCAGAAAGACACUA | UAGUGUCUUUCUGCUCAGG | [1739-1757] ORF |
| 80 | GACUACUUGUAUCUGAACA | UGUUCAGAUACAAGUAGUC | [1670-1688] ORF |
| 81 | CCGAGGUCUCAAUCCAUUG | CAAUGGAUUGAGACCUCGG | [190-208] 5′UTR |
| 82 | ACCUGGUGACUACUUGUAU | AUACAAGUAGUCACCAGGU | [1663-1681] ORF |
| 83 | AGGUGUAUGACAUCGAUGU | ACAUCGAUGUCAUACACCU | [555-573] ORF |
| 84 | UGGGUGACUCAGCAGUUUA | UAAACUGCUGAGUCACCCA | [335-353] ORF |
| 85 | AGAAUUUCUAGCCUCACCU | AGGUGAGGCUAGAAAUUCU | [2535-2553] ORF + 3′UTR |
| 86 | AAGUUCGGCUUCAGAUUUU | AAAAUCUGAAGCCGAACUU | [2510-2528] ORF |
| 87 | GCCAUUGUGAGAAGUUCGG | CCGAACUUCUCACAAUGGC | [2499-2517] ORF |
| 88 | GCAAGAAUGACAUGAAGGC | GCCUUCAUGUCAUUCUUGC | [2289-2307] ORF |
| 89 | GGUGGACUUUAUCUGGAUC | GAUCCAGAUAAAGUCCACC | [2146-2164] ORF |
| 90 | AGAGUAUCAUGUACAGGCA | UGCCUGUACAUGAUACUCU | [2049-2067] ORF |
| 91 | CAGUCAUGUUGUAUCUGGA | UCCAGAUACAACAUGACUG | [225-243] 5′UTR |
| 92 | GUAUGCAGUCAUGUUGUAU | AUACAACAUGACUGCAUAC | [220-238] 5′UTR |
| 93 | GUGUGACAAUGAGAAAGAG | CUCUUUCUCAUUGUCACAC | [1860-1878] ORF |
| 94 | GGUUCUAAGAGGCUGUCGA | UCGACAGCCUCUUAGAACC | [1838-1856] ORF |
| 95 | CGUGGUUCUAAGAGGCUGU | ACAGCCUCUUAGAACCACG | [1835-1853] ORF |
| 96 | AGUGGACAAACAGGCUGUA | UACAGCCUGUUUGUCCACU | [1785-1803] ORF |
| 97 | GACACUAUCUGGCUGCACA | UGUGCAGCCAGAUAGUGUC | [1751-1769] ORF |
| 98 | GUGUGCAUCAUGGAAGUCA | UGACUUCCAUGAUGCACAC | [1583-1601] ORF |
| 99 | AGGUGUUCUAUUGGACUCA | UGAGUCCAAUAGAACACCU | [1434-1452] ORF |
| 100 | GAGGUGUUCUAUUGGACUC | GAGUCCAAUAGAACACCUC | [1433-1451] ORF |
| 101 | CUCCUCCUCAUGUUCAUCU | AGAUGAACAUGAGGAGGAG | [1379-1397] ORF |
| 102 | UCCAGGUGUAUGACAUCGA | UCGAUGUCAUACACCUGGA | [552-570] ORF |
| 103 | UCCGAUAGAAGUGGCACCA | UGGUGCCACUUCUAUCGGA | [456-476] ORF |
| 104 | CGAUUCUUUGCCCUAUUUG | CAAAUAGGGCAAAGAAUCG | [437-455] ORF |
| 105 | UCAGCAGUUUAAGACCAUU | AAUGGUCUUAAACUGCUGA | [343-361] ORF |
| 106 | AGCCCUUAGUCCUUCUAGU | ACUAGAAGGACUAAGGGCU | [134-152] 5′UTR |
| 107 | AGUUCGGCUUCAGAUUUUU | AAAAAUCUGAAGCCGAACU | [2511-2529] ORF |
| 108 | CACUGGGCAAGAAUGACAU | AUGUCAUUCUUGCCGAGUG | [2283-2301] ORF |
| 109 | UGCAGAGUAUCAUGUACAG | CUGUACAUGAUACUCUGCA | [2046-2064] ORF |
| 110 | CUGCAGAGUAUCAUGUACA | UGUACAUGAUACUCUGCAG | [2045-2063] ORF |
| 111 | CUUUUGGAGAAACACAAAU | AUUUGUGUUUCUCCAAAAG | [1907-1925] ORF |
| 112 | AGUGUGACAAUGAGAAAGA | UCUUUCUCAUUGUCACACU | [1859-1877] ORF |
| 113 | GUGGUUCUAAGAGGCUGUC | GACAGCCUCUUAGAACCAC | [1836-1854] ORF |
| 114 | CACUAUCUGGCUGCACAUU | AAUGUGCAGCCAGAUAGUG | [1753-1771] ORF |
| 115 | GCCUGGAAUCUUGUUUUUC | GAAAAACAAGAUUCCAGGC | [1522-1540] ORF |
| 116 | UCCUCCUCAUGUUCAUCUG | CAGAUGAACAUGAGGAGGA | [1380-1398] ORF |
| 117 | UGACAUCGAUGUGUGUGCA | UGCACACACAUCGAUGUCA | [562-580] ORF |
| 118 | GUGUAUGACAUCGAUGUGU | ACACAUCGAUGUCAUACAC | [557-575] ORF |
| 119 | UCUUUGCAGAGCGAUUCUU | AAGAAUCGCUCUGCAAAGA | [426-444] ORF |
| 120 | CCCUUAGUCCUUCUAGUUG | CAACUAGAAGGACUAAGGG | [136-154] 5′UTR |
| 121 | CUCAGCAGUUUAAGACCAU | AUGGUCUUAAACUGCUGAG | [342-360] ORF |
| 122 | GUAUCUGGAAUUGGUGGGU | ACCCACCAAUUCCAGAUAC | [235-253] 5′UTR |
| 123 | CACCAGAAAAGAAAGCAUA | UAUGCUUUCUUUUCUGGUG | [2066-2084] ORF |
| 124 | GUCGAGGAGUGUGACAAUG | CAUUGUCACACUCCUCGAC | [1852-1870] ORF |
| 125 | GAAAGACACUAUCUGGCUG | CAGCCAGAUAGUGUCUUUC | [1747-1765] ORF |
| 126 | UCUUGUUUUUCCUGGAGAA | UUCUCCAGGAAAAACAAGA | [1530-1548] ORF |
| 127 | GCUUCUGCUCAUCUUUCAU | AUGAAAGAUGAGCAGAAGC | [1474-1492] ORF |
| 128 | GUCCUUCUAGUUGCGCUUU | AAAGCGCAACUAGAAGGAC | [142-160] 5′UTR |
| 129 | AGUCCUUCUAGUUGCGCUU | AAGCGCAACUAGAAGGACU | [141-159] 5′UTR |
| 130 | GGAGAAACACAAAUUCUGU | ACAGAAUUUGUGUUUCUCC | [1912-1930] ORF |
| 131 | UCUUCCAGGUGUAUGACAU | AUGUCAUACACCUGGAAGA | [549-567] ORF |
| 132 | GCCCUAUUUGACUCCGAUA | UAUCGGAGUCAAAUAGGGC | [446-464] ORF |
| 133 | CAGUUUAAGACCAUUGCAG | CUGCAAUGGUCUUAAACUG | [347-365] ORF |
| 134 | UGCCCUAUUUGACUCCGAU | AUCGGAGUCAAAUAGGGCA | [445-463] ORF |
| 135 | GGCCAACAAGGAGAAGAAA | UUUCUUCUCCUUGUUGGCC | [2338-2356] ORF |
| 136 | GCCACUUUGAGGUGUUCUA | UAGAACACCUCAAAGUGGC | [1425-1443] ORF |
| 137 | UCCUUCUAGUUGCGCUUUU | AAAAGCGCAACUAGAAGGA | [143-161] 5′UTR |
| 138 | UGGCCAACAAGGAGAAGAA | UUCUUCUCCUUGUUGGCCA | [2337-2355] ORF |
| 139 | GAGAAACACAAAUUCUGUA | UACAGAAUUUGUGUUUCUC | [1913-1931] ORF |
| 140 | CCACCAUUGCUCGCUAUGA | UCAUAGCGAGCAAUGGUGG | [1692-1710] ORF |
| 141 | GGCUCACACUGUGAACUUU | AAAGUUCACAGUGUGAGCC | [1237-1255] ORF |
| 142 | AGCGAUUCUUUGCCCUAUU | AAUAGGGCAAAGAAUCGCU | [435-453] ORF |
| 143 | UGGCUCACACUGUGAACUU | AAGUUCACAGUGUGAGCCA | [1236-1254] ORF |
| 144 | GGAGAAGAAAGACUCCAUC | GAUGGAGUCUUUCUUCUCC | [2347-2365] ORF |
| 145 | GCGGUCUUUCGAGUGGUUU | AAACCACUCGAAAGACCGC | [2176-2194] ORF |
| 146 | ACAGAGACCAGCGGUCUUU | AAAGACCGCUGGUCUCUGU | [2166-2184] ORF |
| 147 | GGAAUCUUGUUUUUCCUGG | CCAGGAAAAACAAGAUUCC | [1526-1544] ORF |
| 148 | GCUUAUCCGAGGUCUCAAU | AUUGAGACCUCGGAUAAGC | [184-202] 5′UTR |
| 149 | CUGUGAACUUUGUACUCCA | UGGAGUACAAAGUUCACAG | [1245-1263] ORF |
| 150 | CGGCUUCAGAUUUUUCCAA | UUGGAAAAAUCUGAAGCCG | [2515-2533] ORF |
| 151 | GGGUUCUUGGUCUCACUGA | UCAGUGAGACCAAGAACCC | [250-268] 5′UTR |
| 152 | GUGGGUUCUUGGUCUCACU | AGUGAGACCAAGAACCCAC | [248-266] 5′UTR |
| 153 | GUGGCCACUUUGAGGUGUU | AACACCUCAAAGUGGCCAC | [1422-1440] ORF |
| 154 | CCAGAACAUCCAGUUCCAC | GUGGAACUGGAUGUUCUGG | [1171-1189] ORF |
| 155 | UCAACUUCGACUGCAGCUU | AAGCUGCAGUCGAAGUUGA | [1077-1095] ORF |
| 156 | CCUUCUAGUUGCGCUUUUG | CAAAAGCGCAACUAGAAGG | [144-162] 5′UTR |
| 157 | CAAGAAUGAAGCCGCAGAC | GUCUGCGGCUUCAUUCUUG | [272-290] 5′UTR |
| 158 | AGGAGAAGAAAGACUCCAU | AUGGAGUCUUUCUUCUCCU | [2346-2364] ORF |
| 159 | UGAGCCUGCUGACUAAACU | AGUUUAGUCAGCAGGCUCA | [2196-2214] ORF |
| 160 | GGUCUUUCGAGUGGUUUGU | ACAAACCACUCGAAAGACC | [2178-2196] ORF |
| 161 | CGAAGGUGUCCAAGACAAC | GUUGUCUUGGACACCUUCG | [2113-2131] ORF |
| 162 | UGGAGAAACACAAAUUCUG | CAGAAUUUGUGUUUCUCCA | [1911-1929] ORF |
| 163 | CAGGCUGGUAUGCAGUCAU | AUGACUGCAUACCAGCCUG | [213-231] 5′UTR |
| 164 | UCAUGUUCAUCUGCUCCAG | CUGGAGCAGAUGAACAUGA | [1386-1404] ORF |
| 165 | CUCCUCAUGUUCAUCUGCU | AGCAGAUGAACAUGAGGAG | [1382-1400] ORF |
| 166 | UCACACUGUGAACUUUGUA | UACAAAGUUCACAGUGUGA | [1240-1258] ORF |
| 167 | GCGCUUUUGCUAUGGCCUU | AAGGCCAUAGCAAAAGCGC | [154-172] 5′UTR |
| 168 | UCCAGUUCCACCAGCUUAU | AUAAGCUGGUGGAACUGGA | [1179-1197] ORF |
| 169 | ACAUCCAGUUCCACCAGCU | AGCUGGUGGAACUGGAUGU | [1176-1194] ORF |
| 170 | UCAAGAAUGAAGCCGCAGA | UCUGCGGCUUCAUUCUUGA | [271-289] 5′UTR |
| 171 | UCAGAUUUUUCCAAGAGAA | UUCUCUUGGAAAAAUCUGA | [2520-2538] ORF |
| 172 | CUGGAAUUGGUGGGUUCUU | AAGAACCCACCAAUUCCAG | [239-257] 5′UTR |
| 173 | AGCUGCAUAUGUACAUGAC | GUCAUGUACAUAUGCAGCU | [2259-2277] ORF |
| 174 | CGGUCUUUCGAGUGGUUUG | CAAACCACUCGAAAGACCG | [2177-2195] ORF |
| 175 | AAGGUGUCCAAGACAACAU | AUGUUGUCUUGGACACCUU | [2115-2133] ORF |
| 176 | GGCUUAUCCGAGGUCUCAA | UUGAGACCUCGGAUAAGCC | [183-201] 5′UTR |
| 177 | CUCAUGUUCAUCUGCUCCA | UGGAGCAGAUGAACAUGAG | [1385-1403] ORF |
| 178 | UCCUCAUGUUCAUCUGCUC | GAGCAGAUGAACAUGAGGA | [1383-1401] ORF |
| 179 | CACACUGUGAACUUUGUAC | GUACAAAGUUCACAGUGUG | [1241-1259] ORF |
| 180 | CUCACACUGUGAACUUUGU | ACAAAGUUCACAGUGUGAG | [1239-1257] ORF |
| 181 | CGCCUAUGAAGCCCUUAGU | ACUAAGGGCUUCAUAGGCG | [125-143] 5′UTR |
| 182 | UGCCUCAACUUCGACUGCA | UGCAGUCGAAGUUGAGGCA | [1073-1091] ORF |
| 183 | CCUUCUUUGCAGAGCGAUU | AAUCGCUCUGCAAAGAAGG | [423-441] ORF |
| 184 | GCAUGUGAAAGAGUCCUUC | GAAGGACUCUUUCACAUGC | [409-427] ORF |
| 185 | AGAAGAAAGACUCCAUCAC | GUGAUGGAGUCUUUCUUCU | [2349-2367] ORF |
| 186 | AGCUCCAUAAGGUGGACUU | AAGUCCACCUUAUGGAGCU | [2136-2154] ORF |
| 187 | AGGUGUCCAAGACAACAUG | CAUGUUGUCUUGGACACCU | [2116-2134] ORF |
| 188 | GUAACAUCAAGUGCUACAU | AUGUAGCACUUGAUGUUAC | [1929-1947] ORF |
| 189 | ACAGGCUGUAUGAGUCCUU | AAGGACUCAUACAGCCUGU | [1794-1812] ORF |
| 190 | CAGUGCUCCUGAGCAGAAA | UUUCUGCUCAGGAGCACUG | [1732-1750] ORF |
| 191 | CCAUUGCUCGCUAUGAGUG | CACUCAUAGCGAGCAAUGG | [1695-1713] ORF |
| 192 | GCAUCAUGGAAGUCAACCU | AGGUUGACUUCCAUGAUGC | [1587-1605] ORF |
| 193 | ACCAGCUUAUGGGCUACGU | ACGUAGCCCAUAAGCUGGU | [1188-1206] ORF |
| 194 | CCAGUUCCACCAGCUUAUG | CAUAAGCUGGUGGAACUGG | [1180-1198] ORF |
| 195 | UCCUACCACUGGACCAGAA | UUCUGGUCCAGUGGUAGGA | [1158-1176] ORF |
| 196 | CUAGUUGCGCUUUUGCUAU | AUAGCAAAAGCGCAACUAG | [148-168] 5′UTR |
| 197 | ACGCCUAUGAAGCCCUUAG | CUAAGGGCUUCAUAGGCGU | [124-142] 5′UTR |
| 198 | CUGCAAGAAUUCAAAGCAG | CUGCUUUGAAUUCUUGCAG | [386-404] ORF |
| 199 | CCUGCAAGAAUUCAAAGCA | UGCUUUGAAUUCUUGCAGG | [385-403] ORF |
| 200 | GGCUUCAGAUUUUUCCAAG | CUUGGAAAAAUCUGAAGCC | [2516-2534] ORF |
| 201 | GUGAGAAGUUCGGCUUCAG | CUGAAGCCGAACUUCUCAC | [2505-2523] ORF |
| 202 | CUGAGAAGAAGGGCAAGGU | ACCUUGCCCUUCUUCUCAG | [2433-2451] ORF |
| 203 | ACAAGGAGAAGAAAGACUC | GAGUCUUUCUUCUCCUUGU | [2343-2361] ORF |
| 204 | GCAUAUGUACAUGACAUCU | AGAUGUCAUGUACAUAUGC | [2263-2281] ORF |
| 205 | CUGCAUAUGUACAUGACAU | AUGUCAUGUACAUAUGCAG | [2261-2279] ORF |
| 206 | GCUUCCUGGAGCUGCAUAU | AUAUGCAGCUCCAGGAAGC | [2250-2268] ORF |
| 207 | CUAAACUGGAGAUGGACCA | UGGUCCAUCUCCAGUUUAG | [2208-2226] ORF |
| 208 | UCGAAGGUGUCCAAGACAA | UUGUCUUGGACACCUUCGA | [2112-2130] ORF |
| 209 | CUGGAUCGAAGGUGUCCAA | UUGGACACCUUCGAUCCAG | [2107-2125] ORF |
| 210 | CCACUUUGAGGUGUUCUAU | AUAGAACACCUCAAAGUGG | [1426-1444] ORF |
| 211 | GGCCACUUUGAGGUGUUCU | AGAACACCUCAAAGUGGCC | [1424-1442] ORF |
| 212 | GCUCCAUAAGGUGGACUUU | AAAGUCCACCUUAUGGAGC | [2137-2155] ORF |
| 213 | AGACAACAUGAAGCUCCAU | AUGGAGCUUCAUGUUGUCU | [2125-2143] ORF |
| 214 | GUGUCCAAGACAACAUGAA | UUCAUGUUGUCUUGGACAC | [2118-2136] ORF |
| 215 | CCCUUUGCUUCCAUUCUGC | GCAGAAUGGAAGCAAAGGG | [2030-2048] ORF |
| 216 | AUGUUCAUCUGCUCCAGUU | AACUGGAGCAGAUGAACAU | [1388-1406] ORF |
| 217 | UCCACCAGCUUAUGGGCUA | UAGCCCAUAAGCUGGUGGA | [1185-1203] ORF |
| 218 | ACCAGAACAUCCAGUUCCA | UGGAACUGGAUGUUCUGGU | [1170-1188] ORF |
| 219 | UGGACCAGAACAUCCAGUU | AACUGGAUGUUCUGGUCCA | [1167-1185] ORF |
| 220 | UGGCUCAAGUCCUACCACU | AGUGGUAGGACUUGAGCCA | [1149-1167] ORF |
| 221 | UCAUGGAGAACCUGACCAU | AUGGUCAGGUUCUCCAUGA | [846-864] ORF |
| 222 | CGAUAGAAGUGGCACCAUC | GAUGGUGCCACUUCUAUCG | [460-478] ORF |
| 223 | CCGAUAGAAGUGGCACCAU | AUGGUGCCACUUCUAUCGG | [459-477] ORF |
| 224 | CAAGAGAAUUUCUAGCCUC | GAGGCUAGAAAUUCUCUUG | [2531-2549] ORF + 3′UTR |
| 225 | GAGAAGUUCGGCUUCAGAU | AUCUGAAGCCGAACUUCUC | [2507-2525] ORF |
| 226 | GUGCAGGUCUUCUUCUGUG | CACAGAAGAAGACCUGCAC | [2450-2468] ORF |
| 227 | UGGAGCUGCAUAUGUACAU | AUGUACAUAUGCAGCUCCA | [2256-2274] ORF |
| 228 | UCCAUAAGGUGGACUUUAU | AUAAAGUCCACCUUAUGGA | [2139-2157] ORF |
| 229 | ACUACUUGUAUCUGAACAU | AUGUUCAGAUACAAGUAGU | [1671-1689] ORF |
| 230 | CCUGGUGACUACUUGUAUC | GAUACAAGUAGUCACCAGG | [1664-1682] ORF |
| 231 | UCACUAUAGACCUGGUGAC | GUCACCAGGUCUAUAGUGA | [1654-1672] ORF |
| 232 | CUGUGGCUCACACUGUGAA | UUCACAGUGUGAGCCACAG | [1233-1251] ORF |
| 233 | AGAACAUCCAGUUCCACCA | UGGUGGAACUGGAUGUUCU | [1173-1191] ORF |
| 234 | UCAAGUCCUACCACUGGAC | GUCCAGUGGUAGGACUUGA | [1153-1171] ORF |
| 235 | CUCAAGUCCUACCACUGGA | UCCAGUGGUAGGACUUGAG | [1152-1170] ORF |
| 236 | AGAGCGAUUCUUUGCCCUA | UAGGGCAAAGAAUCGCUCU | [433-451] ORF |
| 237 | GUCCUUCUUUGCAGAGCGA | UCGCUCUGCAAAGAAGGAC | [421-439] ORF |
| 238 | AAGCAGCUCUGCAUGUGAA | UUCACAUGCAGAGCUGCUU | [399-417] ORF |
| 239 | CAAAGCAGCUCUGCAUGUG | CACAUGCAGAGCUGCUUUG | [397-415] ORF |
| 240 | AGAAUUCAAAGCAGCUCUG | CAGAGCUGCUUUGAAUUCU | [391-409] ORF |
| 241 | GCCUGCAAGAAUUCAAAGC | GCUUUGAAUUCUUGCAGGC | [384-402] ORF |
| 242 | GAAGCCCUUAGUCCUUCUA | UAGAAGGACUAAGGGCUUC | [132-150] 5′UTR |
| 243 | GCCUGCUGACUAAACUGGA | UCCAGUUUAGUCAGCAGGC | [2199-2217] ORF |
| 244 | UCAUGUACAGGCACCAGAA | UUCUGGUGCCUGUACAUGA | [2055-2073] ORF |
| 245 | GUAUCAUGUACAGGCACCA | UGGUGCCUGUACAUGAUAC | [2052-2070] ORF |
| 246 | CUGGUGACUACUUGUAUCU | AGAUACAAGUAGUCACCAG | [1665-1683] ORF |
| 247 | CAAGGUCACUCAUCUCCUC | GAGGAGAUGAGUGACCUUG | [1615-1633] ORF |
| 248 | GGCUUCUGCUCAUCUUUCA | UGAAAGAUGAGCAGAAGCC | [1473-1491] ORF |
| 249 | UGUGGCUUCUGCUCAUCUU | AAGAUGAGCAGAAGCCACA | [1470-1488] ORF |
| 250 | GGUGUUCUAUUGGACUCAC | GUGAGUCCAAUAGAACACC | [1435-1453] ORF |
| 251 | CGCUUUUGCUAUGGCCUUC | GAAGGCCAUAGCAAAAGCG | [155-173] 5′UTR |
| 252 | GGACCAGAACAUCCAGUUC | GAACUGGAUGUUGUGGUCC | [1168-1186] ORF |
| 253 | UGGAGAACCUGACCAUCAG | CUGAUGGUCAGGUUCUCCA | [849-867] ORF |
| 254 | CUCUGCAUGUGAAAGAGUC | GACUCUUUCACAUGCAGAG | [405-423] ORF |
| 255 | CAGAUUUUUCCAAGAGAAU | AUUCUCUUGGAAAAAUCUG | [2521-2539] ORF |
| 256 | UCCUGGCCAACAAGGAGAA | UUCUCCUUGUUGGCCAGGA | [2334-2352] ORF |
| 257 | UGGUUUGUGAGCCUGCUGA | UCAGCAGGCUCACAAACCA | [2189-2207] ORF |
| 258 | UCGAGUGGUUUGUGAGCCU | AGGCUCACAAACCACUCGA | [2184-2202] ORF |
| 259 | GCCUAUGAAGCCCUUAGUC | GACUAAGGGCUUCAUAGGC | [126-144] 5′UTR |
| 260 | GGAGUCAUGGAGAACCUGA | UCAGGUUCUCCAUGACUCC | [842-860] ORF |
| 261 | GGACAAACUCAAAUUCCUC | GAGGAAUUUGAGUUUGUCC | [532-550] ORF |
| 262 | UGGACAAACUCAAAUUCCU | AGGAAUUUGAGUUUGUCCA | [531-549] ORF |
| 263 | GUGAAAGAGUCCUUCUUUG | CAAAGAAGGACUCUUUCAC | [413-431] ORF |
| 264 | AUGUGAAAGAGUCCUUCUU | AAGAAGGACUCUUUCACAU | [411-429] ORF |
| 265 | AGCUCUGCAUGUGAAAGAG | CUCUUUCACAUGCAGAGCU | [403-421] ORF |
| 266 | GAAGUUCGGCUUCAGAUUU | AAAUCUGAAGCCGAACUUC | [2509-2527] ORF |
| 267 | UCCAGAAAGUGGCUGCUGA | UCAGCAGCCACUUUCUGGA | [2418-2436] ORF |
| 268 | UGCAUAUGUACAUGACAUC | GAUGUCAUGUACAUAUGCA | [2262-2280] ORF |
| 269 | GCUGCAUAUGUACAUGACA | UGUCAUGUACAUAUGCAGC | [2260-2278] ORF |
| 270 | CCAUAAGGUGGACUUUAUC | GAUAAAGUCCACCUUAUGG | [2140-2158] ORF |
| 271 | CCAAGACAACAUGAAGCUC | GAGCUUCAUGUUGUCUUGG | [2122-2140] ORF |
| 272 | UGCUUCCAUUCUGCAGAGU | ACUCUGCAGAAUGGAAGCA | [2035-2053] ORF |
| 273 | CAGGCUGUAUGAGUCCUUC | GAAGGACUCAUACAGCCUG | [1795-1813] ORF |
| 274 | UCAUGGAAGUCAACCUCCU | AGGAGGUUGACUUCCAUGA | [1590-1608] ORF |
| 275 | CUGGAAUCUUGUUUUUCCU | AGGAAAAACAAGAUUCCAG | [1524-1542] ORF |
| 276 | CCAACUUCUGGAAGUGGCU | AGCCACUUCCAGAAGUUGG | [1497-1515] ORF |
| 277 | CACUUUGAGGUGUUCUAUU | AAUAGAACACCUCAAAGUG | [1427-1445] ORF |
| 278 | UGGCCACUUUGAGGUGUUC | GAACACCUCAAAGUGGCCA | [1423-1441] ORF |
| 279 | UGUGAAAGAGUCCUUCUUU | AAAGAAGGACUCUUUCACA | [412-430] ORF |
| 280 | GCAGCUCUGCAUGUGAAAG | CUUUCACAUGCAGAGCUGC | [401-419] ORF |
| 281 | AGAAGUUCGGCUUCAGAUU | AAUCUGAAGCCGAACUUCU | [2508-2528] ORF |
| 282 | UGAAGCCCUUAGUCCUUCU | AGAAGGACUAAGGGCUUCA | [131-149] 5′UTR |
| 283 | CAGGUCUUCUUCUGUGGCU | AGCCACAGAAGAAGACCUG | [2453-2471] ORF |
| 284 | UUGACCUCCUGGCCAACAA | UUGUUGGCCAGGAGGUCAA | [2328-2346] ORF |
| 285 | CUCCAUAAGGUGGACUUUA | UAAAGUCCACCUUAUGGAG | [2138-2156] ORF |
| 286 | CAGGAUCUUUGCCUCUGAG | CUCAGAGGCAAAGAUCCUG | [1975-1993] ORF |
| 287 | GGCUGGUAUGCAGUCAUGU | ACAUGACUGCAUACCAGCC | [215-233] 5′UTR |
| 288 | UGAGCAGAAAGACACUAUC | GAUAGUGUCUUUCUGCUCA | [1741-1759] ORF |
| 289 | GUGCUCCUGAGCAGAAAGA | UCUUUCUGCUCAGGAGCAC | [1734-1752] ORF |
| 290 | UGACUACUUGUAUCUGAAC | GUUCAGAUACAAGUAGUCA | [1669-1687] ORF |
| 291 | GACAAACUCAAAUUCCUCU | AGAGGAAUUUGAGUUUGUC | [533-551] ORF |
| 292 | AGCAGCUCUGCAUGUGAAA | UUUCACAUGCAGAGCUGCU | [400-418] ORF |
| 293 | GCUUCAGAUUUUUCCAAGA | UCUUGGAAAAAUCUGAAGC | [2517-2535] ORF |
| 294 | GUACAUGACAUCUGCACUG | CAGUGCAGAUGUCAUGUAC | [2269-2287] ORF |
| 295 | GGAUCUUUGCCUCUGAGCA | UGCUCAGAGGCAAAGAUCC | [1977-1995] ORF |
| 296 | GUGACAAUGAGAAAGAGUC | GACUCUUUCUCAUUGUCAC | [1862-1880] ORF |
| 297 | CUGUCGAGGAGUGUGACAA | UUGUCACACUCCUCGACAG | [1850-1868] ORF |
| 298 | CCUGGAAUCUUGUUUUUCC | GGAAAAACAAGAUUCCAGG | [1523-1541] ORF |
| 299 | UCGUGUGGCUUCUGCUCAU | AUGAGCAGAAGCCACACGA | [1467-1485] ORF |
| 300 | UCCUCUUCCAGGUGUAUGA | UCAUACACCUGGAAGAGGA | [546-564] ORF |
| 301 | AGGUGCAGGUCUUCUUCUG | CAGAAGAAGACCUGCACCU | [2448-2466] ORF |
| 302 | UGACAUCUGCACUGGGCAA | UUGCCCAGUGCAGAUGUCA | [2274-2292] ORF |
| 303 | GAAGCUCCAUAAGGUGGAC | GUCCACCUUAUGGAGCUUC | [2134-2152] ORF |
| 304 | UGAAGCUCCAUAAGGUGGA | UCCACCUUAUGGAGCUUCA | [2133-2151] ORF |
| 305 | UGGUCUCACUGACUUCAAG | CUUGAAGUCAGUGAGACCA | [257-275] 5′UTR |
| 306 | GUGGCUUCUGCUCAUCUUU | AAAGAUGAGCAGAAGCCAC | [1471-1469] ORF |
| 307 | GUCUCACUGACUUCAAGAA | UUCUUGAAGUCAGUGAGAC | [259-277] 5′UTR |
| 308 | UUCUUGGUCUCACUGACUU | AAGUCAGUGAGACCAAGAA | [253-271] 5′UTR |
| 309 | CUUCAGAUUUUUCCAAGAG | CUCUUGGAAAAAUCUGAAG | [2518-2536] ORF |
| 310 | GCCAACAAGGAGAAGAAAG | CUUUCUUCUCCUUGUUGGC | [2339-2357] ORF |
| 311 | GGCACCAGAAAAGAAAGCA | UGCUUUCUUUUCUGGUGCC | [2064-2082] ORF |
| 312 | CUCACUGACUUCAAGAAUG | CAUUCUUGAAGUCAGUGAG | [261-279] 5′UTR |
| 313 | AGAAUGACAUGAAGGCCAU | AUGGCCUUCAUGUCAUUCU | [2292-2310] ORF |
| 314 | CACUGACUUCAAGAAUGAA | UUCAUUCUUGAAGUCAGUG | [263-281] 5′UTR |
| 315 | UUCAGAUUUUUCCAAGAGA | UCUCUUGGAAAAAUCUGAA | [2519-2537] ORF |
| 316 | GGUUCUUGGUCUCACUGAC | GUCAGUGAGACCAAGAACC | [251-269] 5′UTR |
| 317 | GAAUGACAUGAAGGCCAUU | AAUGGCCUUCAUGUCAUUC | [2293-2311] ORF |
| 318 | UCAAAUUCCUCUUCCAGGU | ACCUGGAAGAGGAAUUUGA | [540-558] ORF |
| 319 | CUGCUGAGAAGAAGGGCAA | UUGCCCUUCUUCUCAGCAG | [2430-2448] ORF |
| 320 | AGCAGAAAGACACUAUCUG | CAGAUAGUGUCUUUCUGCU | [1743-1761] ORF |

TABLE H
21-mer oligonucleotides to NADPH oxidase 5 (NOX5)

| | | | | |
|---|---|---|---|---|
| | | | Other | Human-20127623 |
| Number | Sense siRNA | AntiSense siRNA | Sp | ORF: 302-2545 |

| | | | |
|---|---|---|---|
| 1 | UGAGAUACUUUUGGAGAAACA | UGUUUCUCCAAAAGUAUCUCA | [1900-1920] ORF |
| 2 | CCAAGGGCUCUGAGAUACUUU | AAAGUAUCUCAGAGCCCUUGG | [1890-1910] ORF |
| 3 | GAGAUACUUUUGGAGAAACAC | GUGUUUCUCCAAAAGUAUCUC | [1901-1921] ORF |
| 4 | UCUGAGAUACUUUUGGAGAAA | UUUCUCCAAAAGUAUCUCAGA | [1898-1918] ORF |
| 5 | GGGCUCUGAGAUACUUUUGGA | UCCAAAAGUAUCUCAGAGCCC | [1894-1914] ORF |
| 6 | CUGAGAUACUUUUGGAGAAAC | GUUUCUCCAAAAGUAUCUCAG | [1899-1919] ORF |
| 7 | UGGUGCCUGGAAUCUUGUUUU | AAAACAAGAUUCCAGGCACCA | [1518-1538] ORF |
| 8 | CCUGCUGACUAAACUGGAGAU | AUCUCCAGUUUAGUCAGCAGG | [2200-2220] ORF |
| 9 | CAAAUUCUGUAACAUCAAGUG | CACUUGAUGUUACAGAAUUUG | [1921-1941] ORF |
| 10 | GCAGUCAUGUUGUAUCUGGAA | UUCCAGAUACAACAUGACUGC | [224-244] 5′UTR |
| 11 | CUGACUAAACUGGAGAUGGAC | GUCCAUCUCCAGUUUAGUCAG | [2204-2224] ORF |
| 12 | GCUGACUAAACUGGAGAUGGA | UCCAUCUCCAGUUUAGUCAGC | [2203-2223] ORF |
| 13 | GGCUCUGAGAUACUUUUGGAG | CUCCAAAAGUAUCUCAGAGCC | [1895-1915] ORF |
| 14 | GAGCAAGGUGUUCCAGAAAGU | ACUUUCUGGAACACCUUGCUC | [2407-2427] ORF |
| 15 | CUCUGAGAUACUUUUGGAGAA | UUCUCCAAAAGUAUCUCAGAG | [1897-1917] ORF |
| 16 | CAAGGGCUCUGAGAUACUUUU | AAAAGUAUCUCAGAGCCCUUG | [1861-1911] ORF |
| 17 | UGGUAUGCAGUCAUGUUGUAU | AUACAACAUGACUGCAUACCA | [216-238] 5′UTR |
| 18 | AGACACUAUCUGGCUGCACAU | AUGUGCAGCCAGAUAGUGUCU | [1750-1770] ORF |
| 19 | CGUGUGCAUCAUGGAAGUCAA | UUGACUUCCAUGAUGCACACG | [1582-1602] ORF |
| 20 | GCAAGAAUUCAAAGCAGCUCU | AGAGCUGCUUUGAAUUCUUGC | [388-406] ORF |
| 21 | CUGCUGACUAAACUGGAGAUG | CAUCUCCAGUUUAGUCAGCAG | [2201-2221] ORF |
| 22 | GGUGGACUUUAUCUGGAUCAA | UUGAUCCAGAUAAAGUCCACC | [2145-2166] ORF |
| 23 | GCUCUGAGAUACUUUUGGAGA | UCUCCAAAAGUAUCUCAGAGC | [1896-1916] ORF |
| 24 | UGAGAAAGAGUCAAAGGUCGU | ACGACCUUUGACUCUUUCUCA | [1869-1889] ORF |
| 25 | GGAGUGUGACAAUGAGAAAGA | UCUUUCUCAUUGUCACACUCC | [1857-1877] ORF |
| 26 | GGACAAACAGGCUGUAUGAGU | ACUCAUACAGCCUGUUUGUCC | [1786-1808] ORF |
| 27 | GUGGACAAACAGGCUGUAUGA | UCAUACAGCCUGUUUGUCCAC | [1786-1806] ORF |
| 28 | AGACCUGGUGACUACUUGUAU | AUACAAGUAGUCACCAGGUCU | [1661-1681] ORF |
| 29 | CUGGUGCCUGGAAUCUUGUUU | AAACAAGAUUCCAGGCACCAG | [1517-1537] ORF |
| 30 | GAAGGGCCAUUGUGAGAAGUU | AACUUCUCACAAUGGCCCUUC | [2494-2514] ORF |
| 31 | AGUCAUGUUGUAUCUGGAAUU | AAUUCCAGAUACAACAUGACU | [226-248] 5′UTR |
| 32 | AAAGAGUCAAAGGUCGUCCAA | UUGGACGACCUUUGACUCUUU | [1873-1893] ORF |
| 33 | GCUGGUGCCUGGAAUCUUGUU | AACAAGAUUCCAGGCACCAGC | [1516-1536] ORF |
| 34 | CUACCACUGGACCAGAACAUC | GAUGUUCUGGUCCAGUGGUAG | [1160-1180] ORF |
| 35 | CCUACCACUGGACCAGAACAU | AUGUUCUGGUCCAGUGGUAGG | [1159-1179] ORF |
| 36 | CCCAUGGACAAACUCAAAUUC | GAAUUUGAGUUUGUCCAUGGG | [527-547] ORF |
| 37 | GAGAUCAGCCUGCAAGAAUUC | GAAUUCUUGCAGGCUGAUCUC | [377-397] ORF |
| 38 | GCAGAGUAUCAUGUACAGGCA | UGCCUGUACAUGAUACUCUGC | [2047-2067] ORF |
| 39 | CUGGUAUGCAGUCAUGUUGUA | UACAACAUGACUGCAUACCAG | [217-237] 5′UTR |
| 40 | CAGUGGACAAACAGGCUGUAU | AUACAGCCUGUUUGUCCACUG | [1784-1804] ORF |
| 41 | UCAUCUUUCAUGGGCCCAACU | AGUUGGGCCCAUGAAAGAUGA | [1482-1502] ORF |
| 42 | CAAACUCAAAUUCCUCUUCCA | UGGAAGAGGAAUUUGAGUUUG | [535-555] ORF |
| 43 | UCUGCAUGUGAAAGAGUCCUU | AAGGACUCUUUCACAUGCAGA | [406-426] ORF |
| 44 | AGCAAGGUGUUCCAGAAAGUG | CACUUUCUGGAACACCUUGCU | [2408-2428] ORF |
| 45 | GUACAGGCACCAGAAAAGAAA | UUUCUUUUCUGGUGCCUGUAC | [2059-2079] ORF |
| 46 | UGCUACAUCGAUGGGCCUUAU | AUAAGGCCCAUCGAUGUAGCA | [1940-1960] ORF |
| 47 | GGAGAAACACAAAUUCUGUAA | UUACAGAAUUUGUGUUUCUCC | [1912-1932] ORF |
| 48 | GAAAGAGUCAAAGGUCGUCCA | UGGACGACCUUUGACUCUUUC | [1872-1892] ORF |
| 49 | GUAUGCAGUCAUGUUGUAUCU | AGAUACAACAUGACUGCAUAC | [220-240] 5′UTR |
| 50 | GUGUGCAUCAUGGAAGUCAAC | GUUGACUUCCAUGAUGCACAC | [1583-1603] ORF |
| 51 | GGUGCCUGGAAUCUUGUUUUU | AAAAACAAGAUUCCAGGCACC | [1519-1539] ORF |
| 52 | GUGGACUUUAUCUGGAUCAAC | GUUGAUCCAGAUAAAGUCCAC | [2147-2167] ORF |
| 53 | UAAGGUGGACUUUAUCUGGAU | AUCCAGAUAAAGUCCACCUUA | [2143-2163] ORF |
| 54 | CCAGAAAAGAAAGCAUACUUG | CAAGUAUGCUUUCUUUUCUGG | [2068-2088] ORF |
| 55 | GUCAUGUUGUAUCUGGAAUUG | CAAUUCCAGAUACAACAUGAC | [227-247] 5′UTR |
| 56 | UGCAGUCAUGUUGUAUCUGGA | UCCAGAUACAACAUGACUGCA | [223-243] 5′UTR |
| 57 | GGUGUAUGACAUCGAUGUGUG | CACACAUCGAUGUCAUACACC | [556-576] ORF |
| 58 | CAGGUGUAUGACAUCGAUGUG | CACAUCGAUGUCAUACACCUG | [554-574] ORF |
| 59 | CCAGGUGUAUGACAUCGAUGU | ACAUCGAUGUCAUACACCUGG | [553-573] ORF |
| 60 | UCAGCCUGCAAGAAUUCAAAG | CUUUGAAUUCUUGCAGGCUGA | [381-401] ORF |
| 61 | GUGGGUGACUCAGCAGUUUAA | UUAAACUGCUGAGUCACCCAC | [334-354] ORF |
| 62 | GCACUGGGCAAGAAUGACAUG | CAUGUCAUUCUUGCCCAGUGC | [2262-2302] ORF |
| 63 | CAGCGGUCUUUCGAGUGGUUU | AAACCACUCGAAAGACCGCUG | [2174-2194] ORF |
| 64 | AGGUGGACUUUAUCUGGAUCA | UGAUCCAGAUAAAGUCCACCU | [2145-2165] ORF |
| 65 | ACCAGAAAAGAAAGCAUACUU | AAGUAUGCUUUCUUUUCUGGU | [2067-2087] ORF |
| 66 | GCUUCCAUUCUGCAGAGUAUC | GAUACUCUGCAGAAUGGAAGC | [2036-2056] ORF |
| 67 | GCUACAUCGAUGGGCCUUAUG | CAUAAGGCCCAUCGAUGUAGC | [1941-1961] ORF |
| 68 | CAGUCAUGUUGUAUCUGGAAU | AUUCCAGAUACAACAUGACUG | [225-245] 5′UTR |
| 69 | GACAAUGAGAAAGAGUGAAAG | CUUUGACUCUUUCUCAUUGUC | [1864-1884] ORF |
| 70 | CUCCAAGGUCACUCAUCUCCU | AGGAGAUGAGUGACCUUGGAG | [1612-1632] ORF |
| 71 | GAAUCUUGUUUUUCCUGGAGA | UCUCCAGGAAAAACAAGAUUG | [1527-1547] ORF |
| 72 | CUCAUCUUUCAUGGGCCCAAC | GUUGGGCCCAUGAAAGAUGAG | [1481-1501] ORF |
| 73 | GCUCCUCCUCAUGUUCAUCUG | CAGAUGAACAUGAGGAGGAGC | [1378-1398] ORF |
| 74 | GCUGCUCCUCCUCAUGUUCAU | AUGAACAUGAGGAGGAGCAGC | [1375-1395] ORF |
| 75 | AAGCCCUUAGUCCUUCUAGUU | AACUAGAAGGACUAAGGGCUU | [133-153] 5′UTR |
| 76 | CUGGGCAAGAAUGACAUGAAG | CUUCAUGUCAUUCUUGCCCAG | [2285-2305] ORF |
| 77 | GCACCAGAAAAGAAAGCAUAC | GUAUGCUUUCUUUUCUGGUGC | [2065-2085] ORF |
| 78 | CAUUCUGCAGAGUAUCAUGUA | UACAUGAUACUCUGCAGAAUG | [2041-2061] ORF |
| 79 | CCAUUCUGCAGAGUAUCAUGU | ACAUGAUACUCUGCAGAAUGG | [2040-2060] ORF |
| 80 | UCCAUUCUGCAGAGUAUCAUG | CAUGAUACUCUGCAGAAUGGA | [2039-2059] ORF |
| 81 | UGGUUCUAAGAGGCUGUCGAG | CUCGACAGCCUCUUAGAACCA | [1837-1857] ORF |
| 82 | CCUAUGAAGCCCUUAGUCCUU | AAGGACUAAGGGCUUCAUAGG | [127-147] 5′UTR |
| 83 | GAAAGACACUAUCUGGCUGCA | UGCAGCCAGAUAGUGUCUUUC | [1747-1787] ORF |
| 84 | GUGUAUGACAUCGAUGUGUGU | ACACACAUCGAUGUCAUACAC | [557-577] ORF |
| 85 | UCCAGGUGUAUGACAUCGAUG | CAUCGAUGUCAUACACCUGGA | [552-572] ORF |
| 86 | UUCCAGGUGUAUGACAUCGAU | AUCGAUGUCAUACACCUGGAA | [551-571] ORF |
| 87 | GCGAUUCUUUGCCCUAUUUGA | UCAAAUAGGGCAAAGAAUCGC | [436-456] ORF |
| 88 | CCCUUAGUCCUUCUAGUUGCG | CGCAACUAGAAGGACUAAGGG | [136-156] 5′UTR |
| 89 | GGAGAUCAGCCUGCAAGAAUU | AAUUCUUGCAGGCUGAUCUCC | [376-396] ORF |
| 90 | UGAAGGGCCAUUGUGAGAAGU | ACUUCUCACAAUGGCCCUUCA | [2493-2513] ORF |
| 91 | CUGAAGGGCCAUUGUGAGAAG | CUUCUCACAAUGGCCCUUCAG | [2492-2512] ORF |
| 92 | ACUGGGCAAGAAUGACAUGAA | UUCAUGUCAUUCUUGCCCAGU | [2284-2304] ORF |
| 93 | GAGUGUGACAAUGAGAAAGAG | CUCUUUCUCAUUGUCACACUC | [1858-1878] ORF |
| 94 | GACACUAUCUGGCUGCACAUU | AAUGUGCAGCCAGAUAGUGUC | [1751-1771] ORF |
| 95 | CUGCUCCUCCUCAUGUUCAUC | GAUGAACAUGAGGAGGAGCAG | [1376-1396] ORF |
| 96 | UUAGUCCUUCUAGUUGCGCUU | AAGCGCAACUAGAAGGACUAA | [139-159] 5′UTR |
| 97 | CCAUUGUGAGAAGUUCGGCUU | AAGCCGAACUUCUCACAAUGG | [2500-2520] ORF |
| 98 | GUGCUACAUCGAUGGGCCUUA | UAAGGCCCAUCGAUGUAGCAC | [1939-1959] ORF |
| 99 | CCUUUUUUUCACUAUAGACCU | AGGUCUAUAGUGAAAAAAAGG | [1646-1666] ORF |
| 100 | GUGCCUGGAAUCUUGUUUUUC | GAAAAACAAGAUUCCAGGCAC | [1520-1540] ORF |
| 101 | CUCUUCCAGGUGUAUGACAUC | GAUGUCAUACACCUGGAAGAG | [548-568] ORF |
| 102 | GCAAGGUGCAGGUCUUCUUCU | AGAAGAAGACCUGCACCUUGC | [2445-2465] ORF |
| 103 | AAUCUUGUUUUUCCUGGAGAA | UUCUCCAGGAAAAACAAGAUU | [1528-1546] ORF |
| 104 | GCUUUUGCUAUGGCCUUCGUC | GACGAAGGCCAUAGCAAAAGC | [158-176] 5′UTR |
| 105 | GGGAGAUCAGCCUGCAAGAAU | AUUCUUGCAGGCUGAUCUCCC | [375-395] ORF |
| 106 | ACGACGCCUAUGAAGCCCUUA | UAAGGGCUUCAUAGGCGUCGU | [121-141] 5′UTR |
| 107 | CCAACAAGGAGAAGAAAGACU | AGUCUUUCUUCUCCUUGUUGG | [2340-2360] ORF |
| 108 | GGUCACUCAUCUCCUCAUCAA | UUGAUGAGGAGAUGAGUGACC | [1618-1638] ORF |
| 109 | GCCCUAUUUGACUCCGAUAGA | UCUAUCGGAGUCAAAUAGGGC | [446-466] ORF |
| 110 | GGUGGGUGACUCAGCAGUUUA | UAAACUGCUGAGUCACCCACC | [333-353] ORF |
| 111 | CAAGGAGAAGAAAGACUCCAU | AUGGAGUCUUUCUUCUCCUUG | [2344-2364] ORF |
| 112 | GAAGGUGUCCAAGACAACAUG | CAUGUUGUCUUGGACACCUUC | [2114-2134] ORF |
| 113 | CCCUAUUUGACUCCGAUAGAA | UUCUAUCGGAGUCAAAUAGGG | [447-467] ORF |
| 114 | GAGGAGUGUGACAAUGAGAAA | UUUCUCAUUGUCACACUCCUC | [1855-1875] ORF |
| 115 | CGAGGAGUGUGACAAUGAGAA | UUCUCAUUGUCACACUCCUCG | [1854-1874] ORF |
| 116 | GUCACUCAUCUCCUCAUCAAG | CUUGAUGAGGAGAUGAGUGAC | [1619-1839] ORF |
| 117 | AGGUCACUCAUCUCCUCAUCA | UGAUGAGGAGAUGAGUGACCU | [1617-1637] ORF |
| 118 | GGCUUAUCCGAGGUCUCAAUC | GAUUGAGACCUCGGAUAAGCC | [183-203] 5′UTR |
| 119 | UUGCCCUAUUUGACUCCGAUA | UAUCGGAGUCAAAUAGGGCAA | [444-464] ORF |
| 120 | UGGAGAAACACAAAUUCUGUA | UACAGAAUUUGUGUUUCUCCA | [1911-1931] ORF |
| 121 | GGCCACUUUGAGGUGUUCUAU | AUAGAACACCUCAAAGUGGCC | [1424-1444] ORF |
| 122 | GCUCACACUGUGAACUUUGUA | UACAAAGUUCACAGUGUGAGC | [1238-1258] ORF |
| 123 | UGGCUCACACUGUGAACUUUG | CAAAGUUCACAGUGUGAGCCA | [1236-1258] ORF |
| 124 | CCUAUUUGACUCCGAUAGAAG | CUUCUAUCGGAGUCAAAUAGG | [448-468] ORF |
| 125 | AGAAAGUGGCUGCUGAGAAGA | UCUUCUCAGCAGCCACUUUCU | [2421-2441] ORF |
| 126 | CGAAGGUGUCCAAGACAACAU | AUGUUGUCUUGGACACCUUCG | [2113-2133] ORF |
| 127 | CCUCCUCAUGUUCAUCUGCUC | GAGCAGAUGAACAUGAGGAGG | [1381-1401] ORF |
| 128 | GUGAACUUUGUACUCCAGGCU | AGCCUGGAGUACAAAGUUCAC | [1247-1267] ORF |
| 129 | CACUGUGAACUUUGUACUCCA | UGGAGUACAAAGUUCACAGUG | [1243-1283] ORF |
| 130 | CUCAACUUCGACUGCAGCUUC | GAAGCUGCAGUCGAAGUUGAG | [1078-1098] ORF |
| 131 | GUCCUUCUAGUUGCGCUUUUG | CAAAAGCGCAACUAGAAGGAC | [142-162] 5′UTR |
| 132 | AGAGCGAUUCUUUGCCCUAUU | AAUAGGGCAAAGAAUCGCUCU | [433-453] ORF |
| 133 | GAAAGUGGCUGCUGAGAAGAA | UUCUUCUCAGCAGCCACUUUC | [2422-2442] ORF |
| 134 | CUGGCCAACAAGGAGAAGAAA | UUUCUUCUCCUUGUUGGCCAG | [2338-2358] ORF |
| 135 | GGAGCUGCAUAUGUACAUGAC | GUCAUGUACAUAUGCAGCUCC | [2257-2277] ORF |
| 136 | GUAUCUGGAAUUGGUGGGUUC | GAACCCACCAAUUCCAGAUAC | [235-255] 5′UTR |
| 137 | CCAAGACAACAUGAAGCUCCA | UGGAGCUUCAUGUUGUCUUGG | [2122-2142] ORF |
| 138 | UCGUCCAAGGGCUCUGAGAUA | UAUCUCAGAGCCCUUGGACGA | [1886-1906] ORF |
| 139 | UCGAGGAGUGUGACAAUGAGA | UCUCAUUGUCACACUCCUCGA | [1853-1873] ORF |
| 140 | CCACCAUUGCUCGCUAUGAGU | ACUCAUAGCGAGCAAUGGUGG | [1692-1712] ORF |
| 141 | UGGUGAGUACUUGUAUCUGAA | UUCAGAUACAAGUAGUCACCA | [1686-1688] ORF |
| 142 | CGGCUUAUCCGAGGUCUCAAU | AUUGAGACCUCGGAUAAGCCG | [182-202] 5′UTR |
| 143 | CCUCAUGUUCAUCUGCUCCAG | CUGGAGCAGAUGAACAUGAGG | [1384-1404] ORF |
| 144 | UGAACUUUGUACUCCAGGCUC | GAGCCUGGAGUACAAAGUUCA | [1246-1268] ORF |
| 145 | CCAGAACAUCCAGUUCCACCA | UGGUGGAACUGGAUGUUCUGG | [1171-1191] ORF |
| 146 | CAACUUCGACUGCAGCUUCAU | AUGAAGCUGCAGUCGAAGUUG | [1078-1098] ORF |
| 147 | CGGUCUUUCGAGUGGUUUGUG | CACAAACCACUCGAAAGACCG | [2177-2197] ORF |
| 148 | GGACUUUAUCUGGAUCAACAG | CUGUUGAUCCAGAUAAAGUCC | [2149-2169] ORF |
| 149 | CAAGACAACAUGAAGCUCCAU | AUGGAGCUUCAUGUUGUCUUG | [2123-2143] ORF |
| 150 | UCACACUGUGAACUUUGUACU | AGUACAAAGUUCACAGUGUGA | [1240-1260] ORF |
| 151 | GUGGCUCACACUGUGAACUUU | AAAGUUCACAGUGUGAGCCAC | [1236-1255] ORF |
| 152 | UGUGGCUCACACUGUGAACUU | AAGUUCACAGUGUGAGCCACA | [1234-1254] ORF |
| 153 | GCAGAGCGAUUCUUUGCCCUA | UAGGGCAAAGAAUCGCUCUGC | [431-451] ORF |
| 154 | AGACCAUUGCAGGAGAAGAUG | CAUCUUCUCCUGCAAUGGUCU | [354-374] ORF |
| 155 | GCUGCAUAUGUACAUGACAUC | GAUGUCAUGUACAUAUGCAGC | [2260-2280] ORF |
| 156 | GCGGUCUUUCGAGUGGUUUGU | ACAAACCACUCGAAAGACCGC | [2176-2196] ORF |
| 157 | GAGAAACACAAAUUCUGUAAC | GUUACAGAAUUUGUGUUUCUC | [1913-1933] ORF |
| 158 | ACAGGCUGUAUGAGUCCUUCA | UGAAGGACUCAUACAGCCUGU | [1794-1814] ORF |
| 159 | UUAUCCGAGGUCUCAAUCCAU | AUGGAUUGAGACCUCGGAUAA | [186-208] 5′UTR |
| 160 | GCCACUUUGAGGUGUUCUAUU | AAUAGAACACCUCAAAGUGGC | [1425-1445] ORF |
| 161 | CACACUGUGAACUUUGUACUC | GAGUACAAAGUUCACAGUGUG | [1241-1261] ORF |
| 162 | ACAUCCAGUUCCACCAGCUUA | UAAGCUGGUGGAACUGGAUGU | [1176-1196] ORF |
| 163 | GGACCAGAACAUCCAGUUCCA | UGGAACUGGAUGUUCUGGUCC | [1166-1188] ORF |
| 164 | GGACAAACUCAAAUUCCUCUU | AAGAGGAAUUUGAGUUUGUCC | [532-552] ORF |
| 165 | CCUUCUUUGCAGAGCGAUUCU | AGAAUCGCUCUGCAAAGAAGG | [423-443] ORF |
| 166 | CCAAGAGAAUUUCUAGCCUCA | UGAGGCUAGAAAUUCUCUUGG | [2530-2550] ORF + 3′UTR |
| 167 | GGAAUUGGUGGGUUCUUGGUC | GACCAAGAACCCACCAAUUCC | [241-281] 5′UTR |
| 168 | GCCUGCUGACUAAACUGGAGA | UCUCCAGUUUAGUCAGCAGGC | [2199-2219] ORF |
| 169 | CAACAGAGACCAGCGGUCUUU | AAAGACCGCUGGUCUCUGUUG | [2164-2184] ORF |
| 170 | CUGUAACAUCAAGUGCUACAU | AUGUAGCACUUGAUGUUACAG | [1927-1947] ORF |
| 171 | GUCCAAGGGCUCUGAGAUACU | AGUAUCUCAGAGCCCUUGGAC | [1888-1906] ORF |
| 172 | UGACAAUGACAAAGAGUCAAA | UUUGACUCUUUCUCAUUGUCA | [1863-1883] ORF |
| 173 | UGACUACUUGUAUCUGAACAU | AUGUUCAGAUACAAGUAGUCA | [1669-1689] ORF |
| 174 | CUAUAGACCUGGUGACUACUU | AAGUAGUCACCAGGUCUAUAG | [1657-1677] ORF |
| 175 | GAGUGGCCACUUUGAGGUGUU | AACACCUCAAAGUGGCCACUC | [1420-1440] ORF |
| 176 | GGCUCACACUGUGAACUUUGU | ACAAAGUUCACAGUGUGAGCC | [1237-1257] ORF |
| 177 | AACAUCCAGUUCCACCAGCUU | AAGCUGGUGGAACUGGAUGUU | [1175-1195] ORF |
| 178 | GACCAGAACAUCCAGUUCCAC | GUGGAACUGGAUGUUCUGGUC | [1169-1189] ORF |
| 179 | CCUCAACUUCGACUGCAGCUU | AAGCUGCAGUCGAAGUUGAGG | [1075-1095] ORF |
| 180 | GAAUUCAAAGCAGCUCUGCAU | AUGCAGAGCUGCUUUGAAUUC | [392-412] ORF |
| 181 | CAAGAGAAUUUCUAGCCUCAC | GUGAGGCUAGAAAUUCUCUUG | [2531-2551] ORF + 3′UTR |
| 182 | UGAGAAGUUCGGCUUCAGAUU | AAUCUGAAGCCGAACUUCUCA | [2506-2526] ORF |
| 183 | UGCUGAGAAGAAGGGCAAGGU | ACCUUGCCCUUCUUCUCAGCA | [2431-2451] ORF |
| 184 | GGAGAAGAAAGACUCCAUCAC | GUGAUGGAGUCUUUCUUCUCC | [2347-2367] ORF |
| 185 | CCUGGCCAACAAGGAGAAGAA | UUCUUCUCCUUGUUGGCCAGG | [2335-2355] ORF |
| 186 | GUGAGCCUGCUGACUAAACUG | CAGUUUAGUCAGCAGGCUCAC | [2195-2215] ORF |
| 187 | GCUCCAUAAGGUGGACUUUAU | AUAAAGUCCACCUUAUGGAGC | [2137-2157] ORF |
| 188 | GACAACAUGAAGCUCCAUAAG | CUUAUGGAGCUUCAUGUUGUC | [2126-2146] ORF |
| 189 | AGACAACAUGAAGCUCCAUAA | UUAUGGAGCUUCAUGUUGUCU | [2125-2145] ORF |
| 190 | GGUGUCCAAGACAACAUGAAG | CUUCAUGUUGUCUUGGACACC | [2117-2137] ORF |
| 191 | CCAGUGGACAAACAGGCUGUA | UACAGCCUGUUUGUCCACUGG | [1783-1803] ORF |
| 192 | GGUGACUACUUGUAUCUGAAC | GUUCAGAUACAAGUAGUCACC | [1667-1687] ORF |
| 193 | CCAAGGUCACUCAUCUCCUCA | UGAGGAGAUGAGUGACCUUGG | [1614-1634] ORF |
| 194 | GACGCCUAUGAAGCCCUUAGU | ACUAAGGGCUUCAUAGGCGUC | [123-143] 5′UTR |
| 195 | GCAGCUCUGCAUGUGAAAGAG | CUCUUUCACAUGCAGAGCUGC | [401-421] ORF |
| 196 | GCCUGCAAGAAUUCAAAGCAG | CUGCUUUGAAUUCUUGCAGGC | [384-404] ORF |
| 197 | CAGCAGUUUAAGACCAUUGCA | UGCAAUGGUCUUAAACUGCUG | [344-384] ORF |
| 198 | UCUUGGUCUCACUGACUUCAA | UUGAAGUCAGUGAGACCAAGA | [254-274] 5′UTR |
| 199 | UGGAGCAAGGUGUUCCAGAAA | UUUCUGGAACACCUUGCUCCA | [2405-2425] ORF |
| 200 | AGGAGAAGAAAGACUCCAUCA | UGAUGGAGUCUUUCUUCUCCU | [2348-2388] ORF |
| 201 | AGGUGUCCAAGACAACAUGAA | UUCAUGUUGUCUUGGACACCU | [2116-2136] ORF |
| 202 | GGAUCGAAGGUGUCCAAGACA | UGUCUUGGACACCUUCGAUCC | [2109-2129] ORF |
| 203 | AGGCUGGUAUGCAGUCAUGUU | AACAUGACUGCAUACCAGCCU | [214-234] 5′UTR |
| 204 | CCUGGUGACUACUUGUAUCUG | CAGAUACAAGUAGUCACCAGG | [1684-1684] ORF |
| 205 | GGAAUCUUGUUUUUCCUGGAG | CUCCAGGAAAAACAAGAUUCC | [1526-1546] ORF |
| 206 | ACGCCUAUGAAGCCCUUAGUC | GACUAAGGGCUUCAUAGGCGU | [124-144] 5′UTR |
| 207 | CCGAUAGAAGUGGCACCAUCA | UGAUGGUGCCACUUCUAUCGG | [459-479] ORF |
| 208 | GCAUGUGAAAGAGUCCUUCUU | AAGAAGGACUCUUUCACAUGC | [409-429] ORF |
| 209 | AAAGCAGCUCUGCAUGUGAAA | UUUCACAUGCAGAGCUGCUUU | [396-418] ORF |
| 210 | GACUCAGCAGUUUAAGACCAU | AUGGUCUUAAACUGCUGAGUC | [340-360] ORF |
| 211 | GAAGUUCGGCUUCAGAUUUUU | AAAAAUCUGAAGCCGAACUUC | [2509-2529] ORF |
| 212 | AUGAAGCCCUUAGUCCUUCUA | UAGAAGGACUAAGGGCUUCAU | [130-150] 5′UTR |
| 213 | GGUUUGUGAGCCUGCUGACUA | UAGUCAGCAGGCUCACAAACC | [2190-2210] ORF |
| 214 | UCAUGUACAGGCACCAGAAAA | UUUUCUGGUGCCUGUACAUGA | [2055-2075] ORF |
| 215 | AGGCUGUAUGAGUCCUUCAAG | CUUGAAGGACUCAUACAGCCU | [1796-1816] ORF |
| 216 | GUGUGGCUUCUGCUCAUCUUU | AAAGAUGAGCAGAAGCCACAC | [1469-1489] ORF |
| 217 | ACACUGUGGCUCACACUGUGA | UCACAGUGUGAGCCACAGUGU | [1230-1250] ORF |
| 218 | GAACAUCCAGUUCCACCAGCU | AGCUGGUGGAACUGGAUGUUC | [1174-1194] ORF |
| 219 | UGCAUGUGAAAGAGUCCUUCU | AGAAGGACUCUUUCACAUGCA | [408-428] ORF |
| 220 | CUGCAAGAAUUCAAAGCAGCU | AGCUGCUUUGAAUUCUUGCAG | [386-406] ORF |
| 221 | GAGAAGUUCGGCUUCAGAUUU | AAAUCUGAAGCCGAACUUCUC | [2507-2527] ORF |
| 222 | GUGAGAAGUUCGGCUUCAGAU | AUCUGAAGCCGAACUUCUCAC | [2505-2525] ORF |
| 223 | GGAGCAAGGUGUUCCAGAAAG | CUUUCUGGAACACCUUGCUCC | [2408-2426] ORF |
| 224 | UGGAGCUGCAUAUGUACAUGA | UCAUGUACAUAUGCAGCUCCA | [2256-2276] ORF |
| 225 | CUGGAGCUGCAUAUGUACAUG | CAUGUACAUAUGCAGCUCCAG | [2255-2275] ORF |
| 226 | UCCUGGAUCGAAGGUGUCCAA | UUGGACACCUUCGAUCCAGGA | [2105-2125] ORF |
| 227 | GGAUCUUUGCCUCUGAGCAUG | CAUGCUCAGAGGCAAAGAUCC | [1977-1997] ORF |
| 228 | UGAGUCCUUCAAGGCAUCAGA | UCUGAUGCCUUGAAGGACUCA | [1804-1824] ORF |
| 229 | GUAUGAGUCCUUCAAGGCAUC | GAUGCCUUGAAGGACUCAUAC | [1801-1821] ORF |
| 230 | CCUGGAAUCUUGUUUUUCCUG | CAGGAAAAACAAGAUUCCAGG | [1523-1543] ORF |
| 231 | GCCUGGAAUCUUGUUUUUCCU | AGGAAAAACAAGAUUCCAGGC | [1522-1542] ORF |
| 232 | UGCUGGUGCCUGGAAUCUUGU | ACAAGAUUCCAGGCACCAGCA | [1515-1535] ORF |
| 233 | UCAUGGGCCCAACUUCUGGAA | UUCCAGAAGUUGGGCCCAUGA | [1489-1509] ORF |
| 234 | CUAUUGGACUCACCUGUCCUA | UAGGACAGGUGAGUCCAAUAG | [1441-1481] ORF |
| 235 | CCACUUUGAGGUGUUCUAUUG | CAAUAGAACACCUCAAAGUGG | [1426-1446] ORF |
| 236 | CGCUUUUGCUAUGGCCUUCGU | ACGAAGGCCAUAGCAAAAGCG | [159-175] 5′UTR |
| 237 | CAGAACAUCCAGUUCCACCAG | CUGGUGGAACUGGAUGUUCUG | [1172-1192] ORF |
| 238 | CUCCGAUAGAAGUGGCACCAU | AUGGUGCCACUUCUAUCGGAG | [457-477] ORF |
| 239 | GUCCUUCUUUGCAGAGCGAUU | AAUCGCUCUGCAAAGAAGGAC | [421-441] ORF |
| 240 | GAGUCCUUCUUUGCAGAGCGA | UCGCUCUGCAAAGAAGGACUC | [419-439] ORF |
| 241 | GAAAGAGUCCUUCUUUGCAGA | UCUGCAAAGAAGGACUCUUUC | [415-435] ORF |
| 242 | CAGCUCUGCAUGUGAAAGAGU | ACUCUUUCACAUGCAGAGCUG | [402-422] ORF |
| 243 | AGCCUGCAAGAAUUCAAAGCA | UGCUUUGAAUUCUUGCAGGCU | [383-403] ORF |
| 244 | GGGUGACUCAGCAGUUUAAGA | UCUUAAACUGCUGAGUCACCC | [336-356] ORF |
| 245 | AGAGAAUUUCUAGCCUCACCU | AGGUGAGGCUAGAAAUUCUCU | [2533-2553] ORF + 3′UTR |
| 246 | GGCCAACAAGGAGAAGAAAGA | UCUUUCUUCUCCUUGUUGGCC | [2338-2358] ORF |
| 247 | UGACAUCUGCACUGGGCAAGA | UCUUGCCCAGUGCAGAUGUCA | [2274-2294] ORF |
| 248 | GAGCUGCAUAUGUACAUGACA | UGUCAUGUACAUAUGCAGCUC | [2258-2278] ORF |
| 249 | GAGUAUCAUGUACAGGCACCA | UGGUGCCUGUACAUGAUACUC | [2050-2070] ORF |
| 250 | GCUGUCGAGGAGUGUGACAAU | AUUGUCACACUCCUCGACAGC | [1849-1869] ORF |
| 251 | GCUGUAUGAGUCCUUCAAGGC | GCCUUGAAGGACUCAUACAGC | [1798-1818] ORF |
| 252 | CAGGCUGUAUGAGUCCUUCAA | UUGAAGGACUCAUACAGCCUG | [1795-1815] ORF |
| 253 | AAACAGGCUGUAUGAGUCCUU | AAGGACUCAUACAGCCUGUUU | [1792-1812] ORF |
| 254 | GCUGGUAUGCAGUCAUGUUGU | ACAACAUGACUGCAUACCAGC | [216-236] 5′UTR |
| 255 | UCCUGAGCAGAAAGACACUAU | AUAGUGUCUUUCUGCUCAGGA | [1738-1758] ORF |
| 256 | ACCUGGUGACUACUUGUAUCU | AGAUACAAGUAGUCACCAGGU | [1663-1683] ORF |
| 257 | CAAGGUCACUCAUCUCCUCAU | AUGAGGAGAUGAGUGACCUUG | [1615-1635] ORF |
| 258 | AGCCGUGUGCAUCAUGGAAGU | ACUUCCAUGAUGCACACGGCU | [1579-1599] ORF |
| 259 | UGGCUUCUGCUCAUCUUUCAU | AUGAAAGAUGAGCAGAAGCCA | [1472-1492] ORF |
| 260 | CAUGUUCAUCUGCUCCAGUUC | GAACUGGAGCAGAUGAACAUG | [1387-1407] ORF |
| 261 | ACUGGACCAGAACAUCCAGUU | AACUGGAUGUUCUGGUCCAGU | [1165-1185] ORF |
| 262 | AAAUUCCUCUUCCAGGUGUAU | AUACACCUGGAAGAGGAAUUU | [542-562] ORF |
| 263 | GAGCGAUUCUUUGCCCUAUUU | AAAUAGGGCAAAGAAUCGCUC | [434-454] ORF |
| 264 | AGUCCUUCUUUGCAGAGCGAU | AUCGCUCUGCAAAGAAGGACU | [420-440] ORF |
| 265 | CAUGUGAAAGAGUCCUUCUUU | AAAGAAGGACUCUUUCACAUG | [410-430] ORF |
| 266 | CAAAGCAGCUCUGCAUGUGAA | UUCACAUGCAGAGCUGCUUUG | [397-417] ORF |
| 267 | CAAGAAUUCAAAGCAGCUCUG | CAGAGCUGCUUUGAAUUCUUG | [389-409] ORF |
| 268 | UGGGUGACUCAGCAGUUUAAG | CUUAAACUGCUGAGUCACCCA | [335-355] ORF |
| 269 | UCCAAGAGAAUUUCUAGCCUC | GAGGCUAGAAAUUCUCUUGGA | [2529-2549] ORF + 3′UTR |
| 270 | CGGCUUCAGAUUUUUCCAAGA | UCUUGGAAAAAUCUGAAGCCG | [2515-2535] ORF |
| 271 | UCGGCUUCAGAUUUUUCCAAG | CUUGGAAAAAUCUGAAGCCGA | [2514-2534] ORF |
| 272 | UGACUGGAGCAAGGUGUUCCA | UGGAACACCUUGCUCCAGUCA | [2401-2421] ORF |
| 273 | GCAUAUGUACAUGACAUCUGC | GCAGAUGUCAUGUACAUAUGC | [2263-2283] ORF |
| 274 | AGCUGCAUAUGUACAUGACAU | AUGUCAUGUACAUAUGCAGCU | [2259-2279] ORF |
| 275 | GUCUUUCGAGUGGUUUGUGAG | CUCACAAACCACUCGAAAGAC | [2179-2199] ORF |
| 276 | CUCCAUAAGGUGGACUUUAUC | GAUAAAGUCCACCUUAUGGAG | [2138-2158] ORF |
| 277 | AGCUCCAUAAGGUGGACUUUA | UAAAGUCCACCUUAUGGAGCU | [2136-2158] ORF |
| 278 | UGCUUCCAUUCUGCAGAGUAU | AUACUCUGCAGAAUGGAAGCA | [2035-2055] ORF |
| 279 | UUGCUUCCAUUCUGCAGAGUA | UACUCUGCAGAAUGGAAGCAA | [2034-2054] ORF |
| 280 | CCCUUUGCUUCCAUUCUGCAG | CUGCAGAAUGGAAGCAAAGGG | [2030-2050] ORF |
| 281 | GGCUGGUAUGCAGUCAUGUUG | CAACAUGACUGCAUACCAGCC | [215-235] 5′UTR |
| 282 | UCACUAUAGACCUGGUGACUA | UAGUCACCAGGUCUAUAGUGA | [1654-1674] ORF |
| 283 | UGCAUCAUGGAAGUCAACCUC | GAGGUUGACUUCCAUGAUGCA | [1585-1606] ORF |
| 284 | UGGAAUCUUGUUUUUCCUGGA | UCCAGGAAAAACAAGAUUCCA | [1525-1545] ORF |
| 285 | CGUGUGGCUUCUGCUCAUCUU | AAGAUGAGCAGAAGCCACACG | [1468-1488] ORF |
| 286 | GGUGUUCUAUUGGACUCACCU | AGGUGAGUCCAAUAGAACACC | [1435-1455] ORF |
| 287 | CUGGACCAGAACAUCCAGUUC | GAACUGGAUGUUCUGGUCCAG | [1168-1188] ORF |
| 288 | AGUCAUGGAGAACCUGACCAU | AUGGUCAGGUUCUCCAUGACU | [844-864] ORF |
| 289 | GGCUUCAGAUUUUUCCAAGAG | CUCUUGGAAAAAUCUGAAGCC | [2516-2536] ORF |
| 290 | AGAAGUUCGGCUUCAGAUUUU | AAAAUCUGAAGCCGAACUUCU | [2508-2528] ORF |
| 291 | CUGGAGCAAGGUGUUCCAGAA | UUCUGGAACACCUUGCUCCAG | [2404-2424] ORF |
| 292 | CCUGGAGCUGCAUAUGUACAU | AUGUACAUAUGCAGCUCCAGG | [2254-2274] ORF |
| 293 | UCAACAGAGACCAGCGGUCUU | AAGACCGCUGGUCUCUGUUGA | [2163-2183] ORF |
| 294 | CCAUAAGGUGGACUUUAUCUG | CAGAUAAAGUCCACCUUAUGG | [2140-2160] ORF |
| 295 | GAAGCUCCAUAAGGUGGACUU | AAGUCCACCUUAUGGAGCUUC | [2134-2154] ORF |
| 296 | GGCACCAGAAAAGAAAGCAUA | UAUGCUUUCUUUUCUGGUGCC | [2064-2084] ORF |
| 297 | AGGCACCAGAAAAGAAAGCAU | AUGCUUUCUUUUCUGGUGCCU | [2063-2083] ORF |
| 298 | UAACAUCAAGUGCUACAUCGA | UCGAUGUAGCACUUGAUGUUA | [1930-1950] ORF |
| 299 | GCAUCAUGGAAGUCAACCUCC | GGAGGUUGACUUCCAUGAUGC | [1587-1607] ORF |
| 300 | UGAGGUGUUCUAUUGGACUCA | UGAGUCCAAUAGAACACCUCA | [1432-1452] ORF |
| 301 | CCAUGGACAAACUCAAAUUCC | GGAAUUUGAGUUUGUCCAUGG | [528-548] ORF |
| 302 | CGAUUCUUUGCCCUAUUUGAC | GUCAAAUAGGGCAAAGAAUCG | [437-457] ORF |
| 303 | UCAAAGCAGCUCUGCAUGUGA | UCACAUGCAGAGCUGCUUUGA | [396-416] ORF |
| 304 | UGAAGCCCUUAGUCCUUCUAG | CUAGAAGGACUAAGGGCUUCA | [131-151] 5′UTR |
| 305 | CUGCAUAUGUACAUGACAUCU | AGAUGUCAUGUACAUAUGCAG | [2261-2281] ORF |
| 306 | CAACAUGAAGCUCCAUAAGGU | ACCUUAUGGAGCUUCAUGUUG | [2128-2148] ORF |
| 307 | GCUCCUGAGCAGAAAGACACU | AGUGUCUUUCUGCUCAGGAGC | [1736-1758] ORF |
| 308 | GCCUAUGAAGCCCUUAGUCCU | AGGACUAAGGGCUUCAUAGGC | [125-146] 5′UTR |
| 309 | UCGUGUGGCUUCUGCUCAUCU | AGAUGAGCAGAAGCCACACGA | [1487-1487] ORF |
| 310 | UGGCCACUUUGAGGUGUUCUA | UAGAACACCUCAAAGUGGCCA | [1423-1443] ORF |
| 311 | GCUUAUGGGCUACGUGGUAGU | ACUACCACGUAGCCCAUAAGC | [1192-1212] ORF |
| 312 | CAGCUUAUGGGCUACGUGGUA | UACCACGUAGCCCAUAAGCUG | [1190-1210] ORF |
| 313 | GACAAACUCAAAUUCCUCUUC | GAAGAGGAAUUUGAGUUUGUC | [533-553] ORF |
| 314 | CGAUAGAAGUGGCACCAUCAC | GUGAUGGUGCCACUUCUAUCG | [460-480] ORF |
| 315 | AGCGAUUCUUUGCCCUAUUUG | CAAAUAGGGCAAAGAAUCGCU | [435-455] ORF |
| 316 | UUCUUUGCAGAGCGAUUCUUU | AAAGAAUCGCUCUGCAAAGAA | [425-445] ORF |
| 317 | GAGAAUUUCUAGCCUCACCUC | GAGGUGAGGCUAGAAAUUCUC | [2534-2554] ORF + 3′UTR |
| 318 | GCCAACAAGGAGAAGAAAGAC | GUCUUUCUUCUCCUUGUUGGC | [2339-2359] ORF |
| 319 | GGCAAGAAUGACAUGAAGGCC | GGCCUUCAUGUCAUUCUUGCC | [2288-2308] ORF |
| 320 | UGCACUGGGCAAGAAUGACAU | AUGUCAUUCUUGCCCAGUGCA | [2281-2301] ORF |
| 321 | ACAUCUGCACUGGGCAAGAAU | AUUCUUGCCCAGUGCAGAUGU | [2276-2296] ORF |
| 322 | CCUGAGCAGAAAGACACUAUC | GAUAGUGUCUUUCUGCUCAGG | [1739-1759] ORF |
| 323 | CAGGCUGGUAUGCAGUCAUGU | ACAUGACUGCAUACCAGCCUG | [213-233] 5′UTR |
| 324 | GCUCAUCUUUCAUGGGCCCAA | UUGGGCCCAUGAAAGAUGAGC | [1480-1500] ORF |
| 325 | GAGGUGUUCUAUUGGACUCAC | GUGAGUCCAAUAGAACACCUC | [1433-1453] ORF |
| 326 | GUGGCCACUUUGAGGUGUUCU | AGAACACCUCAAAGUGGCCAC | [1422-1442] ORF |
| 327 | CAUCUGCUCCAGUUCCUGCAU | AUGCAGGAACUGGAGCAGAUG | [1393-1413] ORF |
| 328 | CAAAUUCCUCUUCCAGGUGUA | UACACCUGGAAGAGGAAUUUG | [541-561] ORF |
| 329 | GCAGUUUAAGACCAUUGCAGG | CCUGCAAUGGUCUUAAACUGC | [346-366] ORF |
| 330 | GGUCUCACUGACUUCAAGAAU | AUUCUUGAAGUCAGUGAGACC | [258-278] 5′UTR |
| 331 | CUUCAGAUUUUUCCAAGAGAA | UUCUCUUGGAAAAAUCUGAAG | [2518-2538] ORF |
| 332 | GCUUCAGAUUUUUCCAAGAGA | UCUCUUGGAAAAAUCUGAAGC | [2517-2537] ORF |
| 333 | ACAUGACAUCUGCACUGGGCA | UGCCCAGUGCAGAUGUCAUGU | [2271-2291] ORF |
| 334 | AGGAUCUUUGCCUCUGAGCAU | AUGCUCAGAGGCAAAGAUCCU | [1976-1996] ORF |
| 335 | GGCUUCUGCUCAUCUUUCAUG | CAUGAAAGAUGAGCAGAAGCC | [1473-1493] ORF |
| 336 | CCUCUUCCAGGUGUAUGACAU | AUGUCAUACACCUGGAAGAGG | [547-567] ORF |
| 337 | AAGCUCCAUAAGGUGGACUUU | AAAGUCCACCUUAUGGAGCUU | [2135-2155] ORF |
| 338 | CAGAGUAUCAUGUACAGGCAC | GUGCCUGUACAUGAUACUCUG | [2048-2068] ORF |
| 339 | GACUACUUGUAUCUGAACAUC | GAUGUUCAGAUACAAGUAGUC | [1670-1690] ORF |
| 340 | ACUCAGCAGUUUAAGACCAUU | AAUGGUCUUAAACUGCUGAGU | [341-361] ORF |
| 341 | CUCACUGACUUCAAGAAUGAA | UUCAUUCUUGAAGUCAGUGAG | [261-281] 5′UTR |
| 342 | UGGUCUCACUGACUUCAAGAA | UUCUUGAAGUCAGUGAGACCA | [257-277] 5′UTR |
| 343 | GGUUCUUGGUCUCACUGACUU | AAGUCAGUGAGACCAAGAACC | [251-271] 5′UTR |
| 344 | CCAGAAAGUGGCUGCUGAGAA | UUCUCAGCAGCCACUUUCUGG | [2419-2439] ORF |
| 345 | CAACAAGGAGAAGAAAGACUC | GAGUCUUUCUUCUCCUUGUUG | [2341-2361] ORF |
| 346 | CUCAGCAGUUUAAGACCAUUG | CAAUGGUCUUAAACUGCUGAG | [342-362] ORF |
| 347 | GGGUUCUUGGUCUCACUGACU | AGUCAGUGAGACCAAGAACCC | [250-270] 5′UTR |
| 348 | CAAGAAUGACAUGAAGGCCAU | AUGGCCUUCAUGUCAUUCUUG | [2290-2310] ORF |
| 349 | GCAGAAAGACACUAUCUGGCU | AGCCAGAUAGUGUCUUUCUGC | [1744-1764] ORF |
| 350 | AGCAGUGCUCCUGAGCAGAAA | UUUCUGCUCAGGAGCACUGCU | [1730-1750] ORF |
| 351 | UCAUGUUCAUCUGCUCCAGUU | AACUGGAGCAGAUGAACAUGA | [1386-1406] ORF |
| 352 | CAACUUCUGGAAGUGGCUGCU | AGCAGCCACUUCCAGAAGUUG | [1498-1518] ORF |
| 353 | CUCAAAUUCCUCUUCCAGGUG | CACCUGGAAGAGGAAUUUGAG | [539-559] ORF |
| 354 | UCUCACUGACUUCAAGAAUGA | UCAUUCUUGAAGUCAGUGAGA | [260-280] 5′UTR |
| 355 | GACAUCUGCACUGGGCAAGAA | UUCUUGCCCAGUGCAGAUGUC | [2275-2295] ORF |
| 356 | UGGUGGGUUCUUGGUCUCACU | AGUGAGACCAAGAACCCACCA | [246-266] 5′UTR |
| 357 | AAGAAUGACAUGAAGGCCAUU | AAUGGCCUUCAUGUCAUUCUU | [2291-2311] ORF |
| 358 | GCAAGAAUGACAUGAAGGCCA | UGGCCUUCAUGUCAUUCUUGC | [2289-2309] ORF |
| 359 | CUCAUGUUCAUCUGCUCCAGU | ACUGGAGCAGAUGAACAUGAG | [1385-1405] ORF |
| 360 | CUGAGCAGAAAGACACUAUCU | AGAUAGUGUCUUUCUGCUCAG | [1740-1760] ORF |
| 361 | GACUUCAAGAAUGAAGCCGCA | UGCGGCUUCAUUCUUGAAGUC | [267-287] 5′UTR |

TABLE I
19-mer oligomers for DUOX2

| | | | | |
|---|---|---|---|---|
| | | | | Human-132566531 |
| Number | Sense siRNA | AntiSense siRNA | Other Sp | ORF: 204-4850 |

| | | | | |
|---|---|---|---|---|
| 1 | CCCAGACAAUUAUACAAAU | AUUUGUAUAAUUGUCUGGG | | [5002-5020] 3′UTR |
| 2 | ACCCAGACAAUUAUACAAA | UUUGUAUAAUUGUCUGGGU | | [5001-5019] 3′UTR |
| 3 | CUAAUUGACUUCCACAAAA | UUUUGUGGAAGUCAAUUAG | | [5299-5317] 3′UTR |
| 4 | AGAAUAUUGACCCAGACAA | UUGUCUGGGUCAAUAUUCU | | [4992-5010] 3′UTR |
| 5 | CAGUACUCGAUCUCAAUUC | GAAUUGAGAUCGAGUACUG | | [5699-5717] 3′UTR |
| 6 | UCAGUACUCGAUCUCAAUU | AAUUGAGAUCGAGUACUGA | | [5698-5716] 3′UTR |
| 7 | GAAUAUUGACCCAGACAAU | AUUGUCUGGGUCAAUAUUC | | [4993-5011] 3′UTR |
| 8 | CCACUUUCCACAUCUACUU | AAGUAGAUGUGGAAAGUGG | | [3934-3952] ORF |
| 9 | GGGAGGAUUUUCACUUCAU | AUGAAGUGAAAAUCCUCCC | | [2965-2983] ORF |
| 10 | GGAGCUAUUUAGGAAGGCU | AGCCUUCCUAAAUAGCUCC | | [2459-2477] ORF |
| 11 | UGGUCCUACUAAAUAUACU | AGUAUAUUUAGUAGGACCA | | [6121-6139] 3′UTR |
| 12 | CUCUAAUUGACUUCCACAA | UUGUGGAAGUCAAUUAGAG | | [5297-5315] 3′UTR |
| 13 | GCCACGCAGUCAAUGUCUA | UAGACAUUGACUGCGUGGC | | [3634-3652] ORF |
| 14 | CCAACACCACCCUAAACCA | UGGUUUAGGGUGGUGUUGG | | [168-186] 5′UTR |
| 15 | GGAGGACAACAUAGUGGUU | AACCACUAUGUUGUCCUCC | | [1406-1424] ORF |
| 16 | CCUUUUUCAUCGGGCUCUU | AAGAGCCCGAUGAAAAAGG | | [5903-5921] 3′UTR |
| 17 | GGAGGUGAAAAGGUACAAU | AUUGUACCUUUUCACCUCC | | [5250-5268] 3′UTR |
| 18 | GUCUAAUGUUUACCAUGUA | UACAUGGUAAACAUUAGAC | | [2785-2803] ORF |
| 19 | CCCGUCUAAUGUUUACCAU | AUGGUAAACAUUAGACGGG | | [2782-2800] ORF |
| 20 | ACCUCAUCCUGUCCAACAA | UUGUUGGACAGGAUGAGGU | | [2284-2302] ORF |
| 21 | UCAACCUCAUCCUGUCCAA | UUGGACAGGAUGAGGUUGA | | [2281-2299] ORF |
| 22 | AGACAUCUGAACCCAACAC | GUGUUGGGUUCAGAUGUCU | | [156-174] 5′UTR |
| 23 | CAGUUUUGGUCCUACUAAA | UUUAGUAGGACCAAAACUG | | [6115-6133] 3′UTR |
| 24 | CCAGAUAUUCAGUACUCGA | UCGAGUACUGAAUAUCUGG | | [5690-5706] 3′UTR |
| 25 | CCCAAAGUGUCAGCAACUU | AAGUUGCUGACACUUUGGG | | [5518-5536] 3′UTR |
| 26 | UGACCAAGAAUGUAGAGAA | UUCUCUACAUUCUUGGUCA | | [4768-4786] ORF |
| 27 | CUGUUGUCCUGGCCAUUUU | AAAAUGGCCAGGACAACAG | | [3604-3622] ORF |
| 28 | GCCGCAACCUCAUAACCUU | AAGGUUAUGAGGUUGCGGC | | [3508-3526] ORF |
| 29 | CCUUCAUGUUCUCUUAUAU | AUAUAAGAGAACAUGAAGG | | [3475-3493] ORF |
| 30 | GGAGGAUUUUCACUUCAUG | CAUGAAGUGAAAAUCCUCC | | [2966-2984] ORF |
| 31 | ACGAAUUCUUCACCAUGAU | AUCAUGGUGAAGAAUUCGU | | [2836-2854] ORF |
| 32 | UGCCAGAUAUUCAGUACUC | GAGUACUGAAUAUCUGGCA | | [5688-5706] 3′UTR |
| 33 | GGCACCAGGAAACAAAGAU | AUCUUUGUUUCCUGGUGCC | | [5544-5562] 3′UTR |
| 34 | GAGGUGAAAAGGUACAAUA | UAUUGUACCUUUUCACCUC | | [5251-5269] 3′UTR |
| 35 | GGUCCCGGCAAUCAUCUAU | AUAGAUGAUUGCCGGGACC | | [3956-3974] ORF |
| 36 | UCCUUCAUGUUCUCUUAUA | UAUAAGAGAACAUGAAGGA | | [3474-3492] ORF |
| 37 | GAAGACAUCUGAACCCAAC | GUUGGGUUCAGAUGUCUUC | | [154-172] 5′UTR |
| 38 | GCAGUCCCAUCAUCAUCCA | UGGAUGAUGAUGGGACUGC | | [2179-2197] ORF |
| 39 | CAAGAAGCUACAAAAGAAA | UUUCUUUUGUAGCUUCUUG | | [2084-2102] ORF |
| 40 | GCCAGAUAUUCAGUACUCG | CGAGUACUGAAUAUCUGGC | | [5689-5707] 3′UTR |
| 41 | CAAAAUCGAUGCGUUGCUU | AAGCAACGCAUCGAUUUUG | | [5313-5331] 3′UTR |
| 42 | GGAGACUAUGUUCUACAAU | AUUGUAGAACAUAGUCUCC | | [5032-5050] 3′UTR |
| 43 | GGCAUAAAUUUGAGGUGUC | GACACCUCAAAUUUAUGCC | | [4324-4342] ORF |
| 44 | CCGGCAAUCAUCUAUGGAG | CUCCAUAGAUGAUUGCCGG | | [3980-3978] ORF |
| 45 | UGCCCACUUUCCACAUCUA | UAGAUGUGGAAAGUGGGCA | | [3931-3949] ORF |
| 46 | CCAACGUCUUUGUGAAUGA | UCAUUCACAAAGACGUUGG | | [3697-3715] ORF |
| 47 | CCAUCUGUGUUGGCGUGUU | AACACGCCAACACAGAUGG | | [3352-3370] ORF |
| 48 | ACACCACCCUAAACCACAG | CUGUGGUUUAGGGUGGUGU | | [171-189] 5′UTR |
| 49 | CUAUUUAGGAAGGCUGUGA | UCACAGCCUUCCUAAAUAG | | [2463-2481] ORF |
| 50 | GAGCUAUUUAGGAAGGCUG | CAGCCUUCCUAAAUAGCUC | | [2460-2478] RF |
| 51 | GAAGGAGCUAUUUAGGAAG | CUUCCUAAAUAGCUCCUUC | | [2456-2474] ORF |
| 52 | GCGAGAAGGAGCUAUUUAG | CUAAAUAGCUCCUUCUCGC | | [2452-2470] ORF |
| 53 | CCCUGACUGUGCUUGACUU | AAGUCAAGCACAGUCAGGG | | [1954-1972] ORF |
| 54 | GCUACUGGUUUGAGAACAC | GUGUUCUCAAACCAGUAGC | Rat, Mouse | [1750-1768] ORF |
| 55 | CCAUUGUCCUCGACCAGUU | AACUGGUCGAGGACAAUGG | | [1706-1726] ORF |
| 56 | GGGUCUGCAACAACUACUG | CAGUAGUUGUUGCAGACCC | Rat | [1306-1324] ORF |
| 57 | CUGGUGUCUACAUGAGAAA | UUUCUCAUGUAGACACCAG | | [1228-1246] ORF |
| 58 | CCUGGUGUCUACAUGAGAA | UUCUCAUGUAGACACCAGG | | [1227-1245] ORF |
| 59 | CUGCCAGAUAUUCAGUACU | AGUACUGAAUAUCUGGCAG | | [5687-5705] 3′UTR |
| 60 | UCUAAUUGACUUCCACAAA | UUUGUGGAAGUCAAUUAGA | | [5296-5316] 3′UTR |
| 61 | CAUCUCUAAUUGACUUCCA | UGGAAGUCAAUUAGAGAUG | | [5294-5312] 3′UTR |
| 62 | CCAAAGAAGAUAAAGACCA | UGGUCUUUAUCUUCUUUGG | | [5154-5172] 3′UTR |
| 63 | CAGUGCAUGAUGAUUAUAA | UUAUAAUCAUCAUGCACUG | | [5053-5071] 3′UTR |
| 64 | GCAGUGCAUGAUGAUUAUA | UAUAAUCAUCAUGCACUGC | | [5052-5070] 3′UTR |
| 65 | CCAAGAAUGUAGAGAAGGC | GCCUUCUCUACAUUCUUGG | | [4771-4789] ORF |
| 66 | GCAAUGGCUGUGCUGGAUA | UAUCCAGCACAGCCAUUGC | | [4258-4276] ORF |
| 67 | CCAAGGCUUUGAGUACAAG | CUUGUACUCAAAGCCUUGG | | [4079-4097] ORF |
| 68 | GCAAUCAUCUAUGGAGGUG | CACCUCCAUAGAUGAUUGC | | [3963-3981] ORF |
| 69 | GUCUACAUCUUCUCAGUCA | UGACUGAGAAGAUGUAGAC | | [3648-3666] ORF |
| 70 | AGACUUUCCUCAACCGCUA | UAGCGGUUGAGGAAAGUCU | | [3535-3553] ORF |
| 71 | GCAACCUCAUAACCUUCCU | AGGAAGGUUAUGAGGUUGC | | [3511-3529] ORF |
| 72 | CUAAUGUUUACCAUGUAUG | CAUACAUGGUAAACAUUAG | Rat | [2787-2805] ORF |
| 73 | CCAUCAUCAUCCAGCUGCU | AGCAGCUGGAUGAUGAUGG | | [2185-2203] ORF |
| 74 | AGAAGACAUCUGAACCCAA | UUGGGUUCAGAUGUCUUCU | | [153-171] 5′UTR |
| 75 | CUACAAAAGAAACUCAAAG | CUUUGAGUUUCUUUUGUAG | | [2091-2109] ORF |
| 76 | GCUACAAAAGAAACUCAAA | UUUGAGUUUCUUUUGUAGC | | [2090-2108] ORF |
| 77 | GCAACAACUACUGGAUUCG | CGAAUCCAGUAGUUGUUGC | Rat | [1312-1330] ORF |
| 78 | UGCAACAACUACUGGAUUC | GAAUCCAGUAGUUGUUGCA | Rat | [1311-1329] ORF |
| 79 | GGUGUCUACAUGAGAAAUG | CAUUUCUCAUGUAGACACC | | [1230-1248] ORF |
| 80 | ACAAAAUCGAUGCGUUGCU | AGCAACGCAUCGAUUUUGU | | [5312-5330] 3′UTR |
| 81 | CCCACAUCUCUAAUUGACU | AGUCAAUUAGAGAUGUGGG | | [5290-5308] 3′UTR |
| 82 | UGCAGUGCAUGAUGAUUAU | AUAAUCAUCAUGCACUGCA | | [5051-5069] 3′UTR |
| 83 | CCAUCAGAAUCCACCUUAG | CUAAGGUGGAUUCUGAUGG | | [4933-4951] 3′UTR |
| 84 | CCCACUUCAUGCACCACUA | UAGUGGUGCAUGAAGUGGG | | [4819-4837] ORF |
| 85 | GGCUUUGAGUACAAGUCAG | CUGACUUGUACUCAAAGCC | | [4083-4101] ORF |
| 86 | GUCCCGGCAAUCAUCUAUG | CAUAGAUGAUUGCCGGGAC | | [3957-3975] ORF |
| 87 | UGGCCAUCAUGUAUGUCUU | AAGACAUACAUGAUGGCCA | | [3799-3817] ORF |
| 88 | CAACGUCUUUGUGAAUGAU | AUCAUUCACAAAGACGUUG | | [3698-3716] ORF |
| 89 | GCUGUUGUCCUGGCCAUUU | AAAUGGCCAGGACAACAGC | | [3603-3621] ORF |
| 90 | AGAUCGUGCUUACUACUAU | AUAGUAGUAAGCACGAUCU | | [3374-3392] ORF |
| 91 | AGAAGAGGUUUGGCAAAAA | UUUUUGCCAAACCUCUUCU | | [3190-3208] ORF |
| 92 | UGGAAAUGGUAUUAGAGAU | AUCUCUAAUACCAUUUCCA | | [3047-3065] ORF |
| 93 | UGGAGGUGGAAAUGGUAUU | AAUACCAUUUCCACCUCCA | | [3041-3059] ORF |
| 94 | GGAUUUUCACUUCAUGCUG | CAGCAUGAAGUGAAAAUCC | | [2969-2987] ORF |
| 95 | UGACAUGGGAGGAUUUUCA | UGAAAAUCCUCCCAUGUCA | | [2959-2977] ORF |
| 96 | UGAAGAAGGAAGCAGCCAA | UUGGCUGCUUCCUUCUUCA | | [2116-2134] ORF |
| 97 | ACAAAAGAAACUCAAAGAG | CUCUUUGAGUUUCUUUUGU | | [2093-2111] ORF |
| 98 | CCCUUAGUGAGUCUGCUUC | GAAGCAGACUCACUAAGGG | | [2028-2046] ORF |
| 99 | AGCAAAGAUUUUAAGAGGA | UCCUCUUAAAAUCUUUGCU | | [133-151] 5′UTR |
| 100 | UCAGGGCCAUUGUCCUCGA | UCGAGGACAAUGGCACUGA | | [1702-1720] ORF |
| 101 | CUGCAACAACUACUGGAUU | AAUCCAGUAGUUGUUGCAG | Rat | [1310-1328] ORF |
| 102 | UCAGGGUCUGCAACAACUA | UAGUUGUUGCAGACCCUGA | Rat | [1303-1321] ORF |
| 103 | CUCUCAGGGUCUGCAACAA | UUGUUGCAGACCCUGAGAG | Rat | [1300-1318] ORF |
| 104 | CCAGGUCAAGACAUAGAAU | AUUCUAUGUCUUGACCUGG | | [5995-6013] 3′UTR |
| 105 | CCCUGAUAUUCUUGAUGUC | GACAUCAAGAAUAUCAGGG | | [5573-5591] 3′UTR |
| 106 | CACAUCUCUAAUUGACUUC | GAAGUCAAUUAGAGAUGUG | | [5292-5310] 3′UTR |
| 107 | UGGAGGUGAAAAGGUACAA | UUGUACCUUUUCACCUCCA | | [5249-5267] 3′UTR |
| 108 | UCCCAAAGAAGAUAAAGAC | GUCUUUAUCUUCUUUGGGA | | [5152-5170] 3′UTR |
| 109 | AGGAGACUAUGUUCUACAA | UUGUAGAACAUAGUCUCCU | | [5031-5049] 3′UTR |
| 110 | GACCCAGACAAUUAUACAA | UUGUAUAAUUGUCUGGGUC | | [5000-5018] 3′UTR |
| 111 | CCCGGCAAUCAUCUAUGGA | UCCAUAGAUGAUUGCCGGG | | [3959-3977] ORF |
| 112 | GCCCACUUUCCACAUCUAC | GUAGAUGUGGAAAGUGGGC | | [3932-3950] ORF |
| 113 | UCAUCAUCCAUGGCAGCUA | UAGCUGCCAUGGAUGAUGA | Rat, Mouse | [3896-3916] ORF |
| 114 | UCCUGGCCAUCAUGUAUGU | ACAUACAUGAUGGCCAGGA | | [3796-3814] ORF |
| 115 | AGAAGUUCUAUUGGUGGUU | AACCACCAAUAGAACUUCU | | [3733-3751] ORF |
| 116 | CCCAACGUCUUUGUGAAUG | CAUUCACAAAGACGUUGGG | | [3696-3714] ORF |
| 117 | CUUAUAUCUUGCUCACCAU | AUGGUGAGCAAGAUAUAAG | | [3487-3505] ORF |
| 118 | GUGCUUACUACUAUGGCUU | AAGCCAUAGUAGUAAGCAC | | [3379-3397] ORF |
| 119 | AGGAUUUUCACUUCAUGCU | AGCAUGAAGUGAAAAUCCU | | [2968-2986] ORF |
| 120 | UGGGAGGAUUUUCACUUCA | UGAAGUGAAAAUCCUCCCA | | [2964-2982] ORF |
| 121 | CCAGAGAGAAAGGUGAGAG | CUCUCACCUUUCUCUCUGG | | [45-63] 5′UTR |
| 122 | GCUGUUUAGUUCUGAAGAG | CUCUUCAGAACUAAACAGC | | [2357-2375] ORF |
| 123 | GUCAACCUCAUCCUGUCCA | UGGACAGGAUGAGGUUGAC | Rat | [2280-2298] ORF |
| 124 | CGAGAACACAAGAAGCUAC | GUAGCUUCUUGUGUUCUCG | | [2076-2094] ORF |
| 125 | CCAAUGUCUUUGUCUGGCA | UGCCAGACAAAGACAUUGG | | [1873-1891] ORF |
| 126 | ACAUCCGAAAUACCACCCU | AGGGUGGUAUUUCGGAUGU | | [1804-1822] ORF |
| 127 | GAAGCAAAGAUUUUAAGAG | CUCUUAAAAUCUUUGCUUC | | [131-149] 5′UTR |
| 128 | CACAGAAGCAAAGAUUUUA | UAAAAUCUUUGCUUCUGUG | | [127-145] 5′UTR |
| 129 | GUGAUCUCAACCCUAAUGU | ACAUUAGGGUUGAGAUCAC | | [1573-1591] ORF |
| 130 | GGCAAAUUCUCCCGUACAG | CUGUACGGGAGAAUUUGCC | | [1455-1473] ORF |
| 131 | ACUACUGGAUUCGGGAGAA | UUCUCCCGAAUCCAGUAGU | Rat | [1318-1336] ORF |
| 132 | UCUGCAACAACUACUGGAU | AUCCAGUAGUUGUUGCAGA | Rat | [1309-1327] ORF |
| 133 | AGGGUCUGCAACAACUACU | AGUAGUUGUUGCAGACCCU | Rat | [1305-1323] ORF |
| 134 | CUCAGGGUCUGCAACAACU | AGUUGUUGCAGACCCUGAG | Rat | [1302-1320] ORF |
| 135 | CCCUGGUGUCUACAUGAGA | UCUCAUGUAGACACCAGGG | | [1226-1244] ORF |
| 136 | GCUGAGCUGUAAAAUAUUU | AAAUAUUUUACAGCUCAGC | | [5931-5949] 3′UTR |
| 137 | UCCAUACUCAGCUCUUUAA | UUAAAGAGCUGAGUAUGGA | | [5874-5892] 3′UTR |
| 138 | UCGAUGCGUUGCUUUGGUA | UACCAAAGCAACGCAUCGA | | [5318-5336] 3′UTR |
| 139 | GUGAAAAGGUACAAUAGUG | CACUAUUGUACCUUUUCAC | | [5254-5272] 3′UTR |
| 140 | GGUGAAAAGGUACAAUAGU | ACUAUUGUACCUUUUCACC | | [5253-5271] 3′UTR |
| 141 | UCCAUCAGAAUCCACCUUA | UAAGGUGGAUUCUGAUGGA | | [4932-4950] 3′UTR |
| 142 | CCACUUCAUGCACCACUAU | AUAGUGGUGCAUGAAGUGG | | [4820-4838] ORF |
| 143 | GAAUGACCAAGAAUGUAGA | UCUACAUUCUUGGUCAUUC | | [4765-4783] ORF |
| 144 | CCAAAUGCUGUGUAAGAAG | CUUCUUACACAGCAUUUGG | | [4424-4442] ORF |
| 145 | CAAUGGCUGUGCUGGAUAC | GUAUCCAGCACAGCCAUUG | | [4259-4277] ORF |
| 146 | CAAUCAUCUAUGGAGGUGA | UCACCUCCAUAGAUGAUUG | | [3964-3982] ORF |
| 147 | GGCAAUCAUCUAUGGAGGU | ACCUCCAUAGAUGAUUGCC | | [3962-3980] ORF |
| 148 | CGGCAAUCAUCUAUGGAGG | CCUCCAUAGAUGAUUGCCG | | [3961-3979] ORF |
| 149 | CCCACUUUCCACAUCUACU | AGUAGAUGUGGAAAGUGGG | | [3933-3951] ORF |
| 150 | UCAUAACCUUCCUGCGAGA | UCUCGCAGGAAGGUUAUGA | | [3517-3535] ORF |
| 151 | GCUGACAUGGGAGGAUUUU | AAAAUCCUCCCAUGUCAGC | | [2957-2975] ORF |
| 152 | CCAAGGACGAAUUCUUCAC | GUGAAGAAUUCGUCCUUGG | | [2830-2848] ORF |
| 153 | CCGUCUAAUGUUUACCAUG | CAUGGUAAACAUUAGACGG | | [2783-2801] ORF |
| 154 | GUUUAGUUCUGAAGAGGAA | UUCCUCUUCAGAACUAAAC | | [2360-2378] ORF |
| 155 | GCUGCUGUUUAGUUCUGAA | UUCAGAACUAAACAGCAGC | | [2354-2372] ORF |
| 156 | UGCUGCUCAAGAUCCCUAA | UUAGGGAUCUUGAGCAGCA | | [2320-2338] ORF |
| 157 | UUAAGAGGAAAGAAGACAU | AUGUCUUCUUUCCUCUUAA | | [143-161] 5′UTR |
| 158 | GCAGCCCAAUGUCUUUGUC | GACAAAGACAUUGGGCUGC | | [1868-1886] ORF |
| 159 | CAGAAGCAAAGAUUUUAAG | CUUAAAAUCUUUGCUUCUG | | [129-147] 5′UTR |
| 160 | CCUCCAUACUCAGCUCUUU | AAAGAGCUGAGUAUGGAGG | | [5872-5890] 3′UTR |
| 161 | CCCUCCAUACUCAGCUCUU | AAGAGCUGAGUAUGGAGGG | | [5871-5889] 3′UTR |
| 162 | CAGAUAUUCAGUACUCGAU | AUCGAGUACUGAAUAUCUG | | [5691-5709] 3′UTR |
| 163 | CAGCCCUGAUAUUCUUGAU | AUCAAGAAUAUCAGGGCUG | | [5570-5588] 3′UTR |
| 164 | CCAGCCCUGAUAUUCUUGA | UCAAGAAUAUCAGGGCUGG | | [5569-5587] 3′UTR |
| 165 | GAGGGCACCAGGAAACAAA | UUUGUUUCCUGGUGCCCUC | | [5541-5559] 3′UTR |
| 166 | CGAUGCGUUGCUUUGGUAU | AUACCAAAGCAACGCAUCG | | [5319-5337] 3′UTR |
| 167 | GUGUGGAGGUGAAAAGGUA | UACCUUUUCACCUCCACAC | | [5246-5264] 3′UTR |
| 168 | CAAAGAAGAUAAAGACCAC | GUGGUCUUUAUCUUCUUUG | | [5155-5173] 3′UTR |
| 169 | GCAUGAUGAUUAUAAGUCC | GGACUUAUAAUCAUCAUGC | | [5057-5075] 3′UTR |
| 170 | UGCACCACUAUGAGAACUU | AAGUUCUCAUAGUGGUGCA | | [4828-4846] ORF |
| 171 | UGGCAUAAAUUUGAGGUGU | ACACCUCAAAUUUAUGCCA | | [4323-4341] ORF |
| 172 | UCGCCACCUACCAGAACAU | AUGUUCUGGUAGGUGGCGA | | [1072-1090] ORF |
| 173 | GGAGUGGCAUAAAUUUGAG | CUCAAAUUUAUGCCACUCC | | [4319-4337] ORF |
| 174 | CUGCCCACUUUCCACAUCU | AGAUGUGGAAAGUGGGCAG | | [3930-3948] ORF |
| 175 | UCUAUGCCCUGCUCAUCAU | AUGAUGAGCAGGGCAUAGA | | [3886-3904] ORF |
| 176 | ACCUCUACAUCCUGCUCUA | UAGAGCAGGAUGUAGAGGU | | [3871-3889] ORF |
| 177 | CCAUCAUGUAUGUCUUCGC | GCGAAGACAUACAUGAUGG | | [3802-3820] ORF |
| 178 | GGCCAUCAUGUAUGUCUUC | GAAGACAUACAUGAUGGCC | | [3800-3816] ORF |
| 179 | CUGGCCAUCAUGUAUGUCU | AGACAUACAUGAUGGCCAG | | [3798-3816] ORF |
| 180 | GUCCUGGCCAUCAUGUAUG | CAUACAUGAUGGCCAGGAC | | [3795-3813] ORF |
| 181 | GGUCCUGGCCAUCAUGUAU | AUACAUGAUGGCCAGGACC | | [3794-3812] ORF |
| 182 | GAAGUUCUAUUGGUGGUUC | GAACCACCAAUAGAACUUC | | [3734-3752] ORF |
| 183 | CCUUCCUGCGAGAGACUUU | AAAGUCUCUCGCAGGAAGG | | [3523-3541] ORF |
| 184 | CUCAUAACCUUCCUGCGAG | CUCGCAGGAAGGUUAUGAG | | [3516-3534] ORF |
| 185 | CGCAACCUCAUAACCUUCC | GGAAGGUUAUGAGGUUGCG | | [3510-3528] ORF |
| 186 | CCGCAACCUCAUAACCUUC | GAAGGUUAUGAGGUUGCGG | | [3509-3527] ORF |
| 187 | CUCCUUCAUGUUCUCUUAU | AUAAGAGAACAUGAAGGAG | | [3473-3491] ORF |
| 188 | GCUUACUACUAUGGCUUUG | CAAAGCCAUAGUAGUAAGC | | [3381-3399] ORF |
| 189 | UGCUUACUACUAUGGCUUU | AAAGCCAUAGUAGUAAGCA | | [3380-3398] ORF |
| 190 | CGUGCUUACUACUAUGGCU | AGCCAUAGUAGUAAGCACG | | [3378-3396] ORF |
| 191 | GAAGAGGUUUGGCAAAAAG | CUUUUUGCCAAACCUCUUC | | [3191-3209] ORF |
| 192 | AGGUGGAAAUGGUAUUAGA | UCUAAUACCAUUUCCACCU | | [3044-3062] ORF |
| 193 | GAGGUGGAAAUGGUAUUAG | CUAAUACCAUUUCCACCUC | | [3043-3061] ORF |
| 194 | GUAUGACCUGGAUGAGAAU | AUUCUCAUCCAGGUCAUAC | | [2801-2819] ORF |
| 195 | UCCCGUCUAAUGUUUACCA | UGGUAAACAUUAGACGGGA | | [2781-2799] ORF |
| 196 | UGGCUGACAAGGAUGGCAA | UUGCCAUCCUUGUCAGCCA | Mouse | [2692-2710] ORF |
| 197 | AACACAAGAAGCUACAAAA | UUUUGUAGCUUCUUGUGUU | | [2080-2098] ORF |
| 198 | AGAACACAAGAAGCUACAA | UUGUAGCUUCUUGUGUUCU | | [2078-2096] ORF |
| 199 | GCCGAGAACACAAGAAGCU | AGCUUCUUGUGUUCUCGGC | | [2074-2092] ORF |
| 200 | AUUGAAGACAUCCGAAAUA | UAUUUCGGAUGUCUUCAAU | | [1797-1815] ORF |
| 201 | CCUGUUCAGUGCCAUUGUC | GACAAUGGCACUGAACAGG | | [1697-1715] ORF |
| 202 | GGACAACAUAGUGGUUGAA | UUCAACCACUAUGUUGUCC | | [1409-1427] ORF |
| 203 | GCAAGAAGUAGAGCACAGA | UCUGUGCUCUACUUCUUGC | | [114-132] 5′UTR |
| 204 | GGCAAGAAGUAGAGCACAG | CUGUGCUCUACUUCUUGCC | | [113-131] 5′UTR |
| 205 | CCAAUCUGAACAGUACCCA | UGGGUACUGUUCAGAUUGG | | [1339-1357] ORF |
| 206 | GGUUUUCAAAGCUCCCAAG | CUUGGGAGCUUUGAAAACC | | [1281-1299] ORF |
| 207 | ACAAGGGUUUUCAAAGCUC | GAGCUUUGAAAACCCUUGU | | [1276-1294] ORF |
| 208 | AGAAAUGCCAGCUGUCAUU | AAUGACAGCUGGCAUUUCU | | [1242-1260] ORF |
| 209 | UGGUGUCUACAUGAGAAAU | AUUUCUCAUGUAGACACCA | | [1229-1247] ORF |
| 210 | GUCAAGACAUAGAAUGUUU | AAACAUUCUAUGUCUUGAC | | [5999-6017] 3′UTR |
| 211 | GGUCAAGACAUAGAAUGUU | AACAUUCUAUGUCUUGACC | | [5996-6016] 3′UTR |
| 212 | CCAUACUCAGCUCUUUAAC | GUUAAAGAGCUGAGUAUGG | | [5875-5893] 3′UTR |
| 213 | UGAGGGUGAAGGCAAGAAG | CUUCUUGCCUUCACCCUCA | | [103-121] 5′UTR |
| 214 | GGGCACCAGGAAACAAAGA | UCUUUGUUUCCUGGUGCCC | | [5543-5561] 3′UTR |
| 215 | GUGAGGGUGAAGGCAAGAA | UUCUUGCCUUCACCCUCAC | | [102-120] 5′UTR |
| 216 | GGACUGACAUUUGAGGGAG | CUCCCUCAAAUGUCAGUCC | | [5344-5362] 3′UTR |
| 217 | CAAGUGUGGAGGUGAAAAG | CUUUUCACCUCCACACUUG | | [5243-5261] 3′UTR |
| 218 | GACAAGUGUGGAGGUGAAA | UUUCACCUCCACACUUGUC | | [5241-5259] 3′UTR |
| 219 | AGAAAAGGCAGGAGACUAU | AUAGUCUCCUGCCUUUUCU | | [5022-5040] 3′UTR |
| 220 | CUCCAUCAGAAUCCACCUU | AAGGUGGAUUCUGAUGGAG | | [4931-4949] 3′UTR |
| 221 | ACAUUUAUGUCACCCAGCU | AGCUGGGUGACAUAAAUGU | | [4546-4564] ORF |
| 222 | UGGUCUUCAAGUCAUCCUU | AAGGAUGACUUGAAGACCA | | [4399-4417] ORF |
| 223 | CUGGAAGAGUGAGGGUGAA | UUCACCCUCACUCUUCCAG | | [94-112] 5′UTR |
| 224 | UCAUCUAUGGAGGUGACAA | UUGUCACCUCCAUAGAUGA | | [3967-3985] ORF |
| 225 | UGACCCACCACCUCUACAU | AUGUAGAGGUGGUGGGUCA | | [3862-3880] ORF |
| 226 | CCAUGUUCUUUCCGACGUG | CACGUCGGAAAGAACAUGG | | [533-551] ORF |
| 227 | CAACCGCUAUGUGCCUUUU | AAAAGGCACAUAGCGGUUG | | [3545-3563] ORF |
| 228 | GACUUUCCUCAACCGCUAU | AUAGCGGUUGAGGAAAGUC | | [3536-3554] ORF |
| 229 | CAACCUCAUAACCUUCCUG | CAGGAAGGUUAUGAGGUUG | | [3512-3530] ORF |
| 230 | UCUCCUUCAUGUUCUCUUA | UAAGAGAACAUGAAGGAGA | | [3472-3490] ORF |
| 231 | GUCUCCUUCAUGUUCUCUU | AAGAGAACAUGAAGGAGAC | | [3471-3489] ORF |
| 232 | CCAGCGUCUCCUUCAUGUU | AACAUGAAGGAGACGCUGG | | [3466-3484] ORF |
| 233 | CAUGGGAGGAUUUUCACUU | AAGUGAAAAUCCUCCCAUG | | [2962-2960] ORF |
| 234 | AGCUGACAUGGGAGGAUUU | AAAUCCUCCCAUGUCAGCU | | [2956-2974] ORF |
| 235 | GCUAUGACGGCUGGUUUAA | UUAAACCAGCCGUCAUAGC | | [313-331] ORF |
| 236 | GUCCCAUCAUCAUCCAGCU | AGCUGGAUGAUGAUGGGAC | | [2182-2200] ORF |
| 237 | CAAAAGAAACUCAAAGAGA | UCUCUUUGAGUUUCUUUUG | | [2094-2112] ORF |
| 238 | ACACAAGAAGCUACAAAAG | CUUUUGUAGCUUCUUGUGU | | [2081-2099] ORF |
| 239 | GAGAACACAAGAAGCUACA | UGUAGCUUCUUGUGUUCUC | | [2077-2095] ORF |
| 240 | CCGAGAACACAAGAAGCUA | UAGCUUCUUGUGUUCUCGG | | [2075-2093] ORF |
| 241 | GACUGUGCUUGACUUCUUU | AAAGAAGUCAAGCACAGUC | | [1958-1976] ORF |
| 242 | CAAUGUCUUUGUCUGGCAU | AUGCCAGACAAAGACAUUG | | [1874-1892] ORF |
| 243 | CCCUGUUCAGUGCCAUUGU | ACAAUGGCACUGAACAGGG | | [1696-1714] ORF |
| 244 | CUAAAAAGCUGAGCUGUAA | UUACAGCUCAGCUUUUUAG | Rat | [5924-5942] 3′UTR |
| 245 | UGCAUCUACUGGAGUGGUC | GACCACUCCAGUAGAUGCA | | [5835-5853] 3′UTR |
| 246 | CCUCUCUGCAUCUACUGGA | UCCAGUAGAUGCAGAGAGG | | [5629-5647] 3′UTR |
| 247 | UGCGUUGCUUUGGUAUUUG | CAAAUACCAAAGCAACGCA | | [5322-5340] 3′UTR |
| 248 | GUGGAGGUGAAAAGGUACA | UGUACCUUUUCACCUCCAC | | [5248-5266] 3′UTR |
| 249 | ACAAGUGUGGAGGUGAAAA | UUUUCACCUCCACACUUGU | | [5242-5260] 3′UTR |
| 250 | UGCAUGAUGAUUAUAAGUC | GACUUAUAAUCAUCAUGCA | | [5056-5074] 3′UTR |
| 251 | AGUGCAUGAUGAUUAUAAG | CUUAUAAUCAUCAUGCACU | | [5054-5072] 3′UTR |
| 252 | CAAAUGAGAAAAGGCAGGA | UCCUGCCUUUUCUCAUUUG | | [5018-5034] 3′UTR |
| 253 | UGCCUUCUCUUCUGUGCAC | GUGCACAGAAGAGAAGGCA | | [4884-4902] 3′UTR |
| 254 | CCACUAUGAGAACUUCUGA | UCAGAAGUUCUCAUAGUGG | | [4832-4850] ORF |
| 255 | AGAAUGUAGAGAAGGCCUG | CAGGCCUUCUCUACAUUCU | | [4774-4792] ORF |
| 256 | ACAUCGCUGUGUAUGAGUG | CACUCAUACACAGCGAUGU | | [1087-1105] ORF |
| 257 | GUGGCAUAAAUUUGAGGUG | CACCUCAAAUUUAUGCCAC | | [4322-4340] ORF |
| 258 | GAGUGACCUACCUGCAAUU | AAUUGCAGGUAGGUCACUC | | [4051-4069] ORF |
| 259 | CCUCUACAUCCUGCUCUAU | AUAGAGCAGGAUGUAGAGG | | [3872-3890] ORF |
| 260 | GCCAUCAUGUAUGUCUUCG | CGAAGACAUACAUGAUGGC | | [3801-3819] ORF |
| 261 | AGGUAUGACAGGUGUGCUU | AAGCACACCUGUCAUACCU | | [3767-3785] ORF |
| 262 | CAGAAGUUCUAUUGGUGGU | ACCACCAAUAGAACUUCUG | | [3732-3750] ORF |
| 263 | UCUUAUAUCUUGCUCACCA | UGGUGAGCAAGAUAUAAGA | | [3486-3504] ORF |
| 264 | CAGAUCGUGCUUACUACUA | UAGUAGUAAGCACGAUCUG | | [3373-3391] ORF |
| 265 | GCAAUCUUCUCGGCCAUCU | AGAUGGCCGAGAAGAUUGC | | [3339-3357] ORF |
| 266 | AGGCGCUGCAAGAGAAGAU | AUCUUCUCUUGCAGCGCCU | | [3241-3259] ORF |
| 267 | CAGAGAGAAAGGUGAGAGU | ACUCUCACCUUUCUCUCUG | | [46-64] 5′UTR |
| 268 | UGUUCUCUCUGGCUGACAA | UUGUCAGCCAGAGAGAACA | | [2683-2701] ORF |
| 269 | ACAUGUUUGUGGAGUCCAU | AUGGACUCCACAAACAUGU | Mouse | [2665-2683] ORF |
| 270 | CUGCUGUUUAGUUCUGAAG | CUUCAGAACUAAACAGCAG | | [2355-2373] ORF |
| 271 | GAAGGCAAGAAGUAGAGCA | UGCUCUACUUCUUGCCUUC | | [110-128] 5′UTR |
| 272 | AGGGUGAAGGCAAGAAGUA | UACUUCUUGCCUUCACCCU | | [105-123] 5′UTR |
| 273 | CCUAGAAGCAACCACUGAU | AUCAGUGGUUGCUUCUAGG | | [6079-6097] 3′UTR |
| 274 | AAGCUGAGCUGUAAAAUAU | AUAUUUUACAGCUCAGCUU | | [5929-5947] 3′UTR |
| 275 | CUGAUAUUCUUGAUGUCAC | GUGACAUCAAGAAUAUCAG | | [5575-5593] 3′UTR |
| 276 | AGCCCUGAUAUUCUUGAUG | CAUCAAGAAUAUCAGGGCU | | [5571-5589] 3′UTR |
| 277 | CUGUGAUGCUUAGAACCUG | CAGGUUCUAAGCAUCACAG | | [5415-5433] 3′UTR |
| 278 | AUGCGUUGCUUUGGUAUUU | AAAUACCAAAGCAACGCAU | | [5321-5339] 3′UTR |
| 279 | CAGGAGACUAUGUUCUACA | UGUAGAACAUAGUCUCCUG | | [5030-5048] 3′UTR |
| 280 | UCGCUGUGUAUGAGUGGCU | AGCCACUCAUACACAGCGA | | [1090-1108] ORF |
| 281 | UCAUGUAUGUCUUCGCCUC | GAGGCGAAGACAUACAUGA | | [3805-3823] ORF |
| 282 | CAGUCAAUGUCUACAUCUU | AAGAUGUAGACAUUGACUG | | [3640-3658] ORF |
| 283 | ACGGUGUGUAUCAGGCUCU | AGAGCCUGAUACACACCGU | | [403-421] ORF |
| 284 | ACAAGCGCUUCGUGGAGAA | UUCUCCACGAAGCGCUUGU | | [3295-3313] ORF |
| 285 | CAAGAGAAGAUGCAGCGAG | CUCGCUGCAUCUUCUCUUG | | [3249-3267] ORF |
| 286 | GGACUGAAGAAGAGGUUUG | CAAACCUCUUCUUCAGUCC | | [3183-3201] ORF |
| 287 | ACCUGGUGCUGCUGUUUAG | CUAAACAGCAGCACCAGGU | | [2347-2365] ORF |
| 288 | UGACCUGGUGCUGCUGUUU | AAACAGCAGCACCAGGUCA | | [2345-2363] ORF |
| 289 | UCACCAUCAUUGCUCUCUG | CAGAGAGCAAUGAUGGUGA | | [2002-2020] ORF |
| 290 | GCCAUCACCAUCAUUGCUC | GAGCAAUGAUGGUGAUGGC | | [1996-2016] ORF |
| 291 | GUCUUUGUCUGGCAUAAAG | CUUUAUGCCAGACAAAGAC | | [1878-1896] ORF |
| 292 | AGCCCAAUGUCUUUGUCUG | CAGACAAAGACAUUGGGCU | | [1870-1888] ORF |
| 293 | GCAAAUUCUCCCGUACAGA | UCUGUACGGGAGAAUUUGC | | [1456-1474] ORF |
| 294 | CAAGAAGUAGAGCACAGAA | UUCUGUGCUCUACUUCUUG | | [115-133] 5′UTR |
| 295 | CAGGUCAAGACAUAGAAUG | CAUUCUAUGUCUUGACCUG | | [5996-6014] 3′UTR |
| 296 | AGCUGAGCUGUAAAAUAUU | AAUAUUUUACAGCUCAGCU | | [5930-5948] 3′UTR |
| 297 | CCUAAAAAGCUGAGCUGUA | UACAGCUCAGCUUUUUAGG | | [5923-5941] 3′UTR |
| 298 | CUCCAUACUCAGCUCUUUA | UAAAGAGCUGAGUAUGGAG | | [5873-5891] 3′UTR |
| 299 | UGCUUGGACUGACAUUUGA | UCAAAUGUCAGUCCAAGCA | | [5339-5357] 3′UTR |
| 300 | GCUUUGGUAUUUGCUUGGA | UCCAAGCAAAUACCAAAGC | | [5328-5346] 3′UTR |
| 301 | AAAAGGUACAAUAGUGCUU | AAGCACUAUUGUACCUUUU | | [5257-5275] 3′UTR |
| 302 | ACCACUAUGAGAACUUCUG | CAGAAGUUCUCAUAGUGGU | | [4831-4849] ORF |
| 303 | CCUUCUUCAACUCCCUGCA | UGCAGGGAGUUGAAGAAGG | | [4893-4711] ORF |
| 304 | CUGGUCUUCAAGUCAUCCU | AGGAUGACUUGAAGACCAG | | [4396-4416] ORF |
| 305 | UGACCUACCUGCAAUUCCA | UGGAAUUGCAGGUAGGUCA | | [4054-4072] ORF |
| 306 | UCUCUUAUAUCUUGCUCAC | GUGAGCAAGAUAUAAGAGA | | [3484-3502] ORF |
| 307 | UCGUGUGUGUGGCAAUCUU | AAGAUUGCCACACACACGA | | [3328-3346] ORF |
| 308 | AAGAAGAGGUUUGGCAAAA | UUUUGCCAAACCUCUUCUU | | [3189-3207] ORF |
| 309 | GAAGAAGAGGUUUGGCAAA | UUUGCCAAACCUCUUCUUC | | [3188-3206] ORF |
| 310 | CUGUGUCAAAGGUGGAGGU | ACCUCCACCUUUGACACAG | | [3023-3041] ORF |
| 311 | CUGGUGGUCUUCAUGAAAG | CUUUCAUGAAGACCACCAG | Mouse | [2745-2763] ORF |
| 312 | CUGGUGCUGCUGUUUAGUU | AACUAAACAGCAGCACCAG | | [2349-2367] ORF |
| 313 | GUUUUGCCAUCACCAUCAU | AUGAUGGUGAUGGCAAAAC | | [1993-2011] ORF |
| 314 | UGCAGCCCAAUGUCUUUGU | ACAAAGACAUUGGGCUGCA | | [1867-1885] ORF |
| 315 | AGAUUUCGGAGUUGGAGGA | UCCUCCAACUCCGAAAUCU | | [1393-1411] ORF |
| 316 | CCCAAUCUGAACAGUACCC | GGGUACUGUUCAGAUUGGG | | [1338-1356] ORF |
| 317 | CUAGAAGCAACCACUGAUG | CAUCAGUGGUUGCUUCUAG | | [6080-6098] 3′UTR |
| 318 | UCAAGACAUAGAAUGUUUC | GAAACAUUCUAUGUCUUGA | | [6000-6018] 3′UTR |
| 319 | GUGAUGCUUAGAACCUGGA | UCCAGGUUCUAAGCAUCAC | | [5417-5435] 3′UTR |
| 320 | CACUAUGAGAACUUCUGAG | CUCAGAAGUUCUCAUAGUG | | [4833-4851] ORF + 3′UTR |
| 321 | CUAUGGAGGUGACAAGCUG | CAGCUUGUCACCUCCAUAG | | [3971-3989] ORF |
| 322 | CACCUCUACAUCCUGCUCU | AGAGCAGGAUGUAGAGGUG | | [3870-3888] ORF |
| 323 | ACCAUGUUCUUUCCGACGU | ACGUCGGAAAGAACAUGGU | | [532-550] ORF |
| 324 | AGAGAAAGGUGAGAGUGCA | UGCACUCUCACCUUUCUCU | | [49-67] 5′UTR |
| 325 | ACAUCGUGUGUGUGGCAAU | AUUGCCACACACACGAUGU | | [3325-3343] ORF |
| 326 | GAGUCUAUGUUCCGGGAGU | ACUCCCGGAACAUAGACUC | Mouse | [2916-2934] ORF |
| 327 | GUGGUCUUCAUGAAAGGCU | AGCCUUUCAUGAAGACCAC | Mouse | [2748-2768] ORF |
| 328 | UGUUUGUGGAGUCCAUGUU | AACAUGGACUCCACAAACA | Rat, Mouse | [2668-2686] ORF |
| 329 | CAGACACCUUUUUGCUCAG | CUGAGCAAAAAGGUGUCUG | | [2519-2537] ORF |
| 330 | UCAGACACCUUUUUGCUCA | UGAGCAAAAAGGUGUCUGA | | [2518-2538] ORF |
| 331 | GCCUUCCCUUAGUGAGUCU | AGACUCACUAAGGGAAGGC | | [2023-2041] ORF |
| 332 | CUGUGCUUGACUUCUUUGA | UCAAAGAAGUCAAGCACAG | | [1960-1978] ORF |
| 333 | CUCCCGUACAGACUAUGUG | CACAUAGUCUGUACGGGAG | | [1463-1481] ORF |
| 334 | CCAGAUUUCGGAGUUGGAG | CUCCAACUCCGAAAUCUGG | | [1391-1409] ORF |
| 335 | GAGAAAUGCCAGCUGUCAU | AUGACAGCUGGCAUUUCUC | | [1241-1259] ORF |
| 336 | AAAAAGCUGAGCUGUAAAA | UUUUACAGCUCAGCUUUUU | | [5926-5944] 3′UTR |
| 337 | CUUGAUGUCACCAGUGAUA | UAUCACUGGUGACAUCAAG | | [5583-5601] 3′UTR |
| 338 | CCUGAUAUUCUUGAUGUCA | UGACAUCAAGAAUAUCAGG | | [5574-5592] 3′UTR |
| 339 | UGGACUGACAUUUGAGGGA | UCCCUCAAAUGUCAGUCCA | | [5343-5381] 3′UTR |
| 340 | GUAUUUGCUUGGACUGACA | UGUCAGUCCAAGCAAAUAC | | [5334-5352] 3′UTR |
| 341 | AAGUCCACCUGUUUAUCAA | UUGAUAAACAGGUGGACUU | | [5070-5068] 3′UTR |
| 342 | AGAGUGAGGGUGAAGGCAA | UUGCCUUCACCCUCACUCU | | [99-117] 5′UTR |
| 343 | UGAUUAUAAGUCCACCUGU | ACAGGUGGACUUAUAAUCA | | [5083-5081] 3′UTR |
| 344 | CUUCUCUUCUGUGCACCUA | UAGGUGCACAGAAGAGAAG | | [4887-4905] 3′UTR |
| 345 | AGCCCUUCUUCAACUCCCU | AGGGAGUUGAAGAAGGGCU | | [4690-4708] ORF |
| 346 | AGAAAGUGCUGAACCGGAG | CUCCGGUUCAGCACUUUCU | | [4621-4639] ORF |
| 347 | CCUUUGCCUCCAUCCUCAA | UUGAGGAUGGAGGCAAAGG | Rat, Mouse | [4375-4393] ORF |
| 348 | CCUUGAUGGACCGUUUGGA | UCCAAACGGUCCAUCAAGG | | [4289-4307] ORF |
| 349 | CUACCAUGUUCUUUCCGAC | GUCGGAAAGAACAUGGUAG | | [530-548] ORF |
| 350 | GCUACCAUGUUCUUUCCGA | UCGGAAAGAACAUGGUAGC | | [529-547] ORF |
| 351 | UUGUGAAUGAUGGGUCCAA | UUGGACCCAUCAUUCACAA | Rat | [3706-3724] ORF |
| 352 | GUGUGUAUCAGGCUCUGGA | UCCAGAGCCUGAUACACAC | | [406-424] ORF |
| 353 | GCUGUCGAGUCUCGUUCAU | AUGAACGAGACUCGACAGC | | [3085-3103] ORF |
| 354 | UGGUCUUCAUGAAAGGCUC | GAGCCUUUCAUGAAGACCA | Mouse | [2749-2767] ORF |
| 355 | CCAGGACAUGUUUGUGGAG | CUCCACAAACAUGUCCUGG | Mouse | [2660-2678] ORF |
| 356 | UGGUGCUGCUGUUUAGUUC | GAACUAAACAGCAGCACCA | | [2350-2368] ORF |
| 357 | UCAUUGCUCUCUGCUGCCU | AGGCAGCAGAGAGCAAUGA | | [2008-2026] ORF |
| 358 | UGUCUUUGUCUGGCAUAAA | UUUAUGCCAGACAAAGACA | | [1877-1895] ORF |
| 359 | AUGUCUUUGUCUGGCAUAA | UUAUGCCAGACAAAGAGAU | | [1876-1894] ORF |
| 360 | CCUGCAGCCCAAUGUCUUU | AAAGACAUUGGGCUGCAGG | | [1865-1883] ORF |
| 361 | AGGAGGUGAAUGAGCUGCU | AGCAGCUCAUUCACCUCCU | | [1357-1375] ORF |
| 362 | CAGCUGUCAUUUCCGGAAG | CUUCCGGAAAUGACAGCUG | | [1250-1268] ORF |
| 363 | UCGGGCUCUUUCCUAAAAA | UUUUUAGGAAAGAGCCCGA | | [5912-5930] 3′UTR |
| 364 | GGUAUUUGCUUGGACUGAC | GUCAGUCCAAGCAAAUACC | | [5333-5351] 3′UTR |
| 365 | AAAGGCAGGAGACUAUGUU | AACAUAGUCUCCUGCCUUU | | [5025-5043] 3′UTR |
| 366 | GGAGAAUAUUGACCCAGAC | GUCUGGGUCAAUAUUCUCC | | [4990-5008] 3′UTR |
| 367 | UGAAGAAGAGGUUUGGCAA | UUGCCAAACCUCUUCUUCA | | [3187-3205] ORF |
| 368 | CUGAAGAAGAGGUUUGGCA | UGCCAAACCUCUUCUUCAG | | [3186-3204] ORF |
| 369 | GGAGCUCUUCUGACUGGAU | AUCCAGUCAGAAGAGCUCC | | [240-258] ORF |
| 370 | CAGGACAUGUUUGUGGAGU | ACUCCACAAACAUGUCCUG | Mouse | [2661-2679] ORF |
| 371 | GGAAUUUGUGGUGGCCUCU | AGAGGCCACCACAAAUUCC | | [1181-1199] ORF |
| 372 | AGGCAGGAGACUAUGUUCU | AGAACAUAGUCUCCUGCCU | | [5027-5045] 3′UTR |
| 373 | GAGAACUUCUGAGCCUGUC | GACAGGCUCAGAAGUUCUC | | [4839-4857] ORF + 3′UTR |
| 374 | UGGAGAUCAGCGUGGUGAA | UUCACCACGCUGAUCUCCA | | [4012-4030] ORF |
| 375 | ACAUUGCACAGACCACCCU | AGGGUGGUCUGUGCAAUGU | | [3415-3433] ORF |
| 376 | UGGAGGUGGAGGUGGAAAU | AUUUCCACCUCCACCUCCA | | [3035-3053] ORF |
| 377 | CGGAGUUGGAGGACAACAU | AUGUUGUCCUCCAACUCCG | | [1399-1417] ORF |
| 378 | UCCACAAAAUCGAUGCGUU | AACGCAUCGAUUUUGUGGA | | [5309-5327] 3′UTR |
| 379 | GGGAGAAUAUUGACCCAGA | UCUGGGUCAAUAUUCUCCC | | [4989-5007] 3′UTR |
| 380 | CCAGGAAUGACCAAGAAUG | CAUUCUUGGUCAUUCCUGG | | [4761-4779] ORF |
| 381 | UCGUUCAUCACUCGGACAC | GUGUCCGAGUGAUGAACGA | | [3096-3114] ORF |
| 382 | GGUGGAGGUGGAAAUGGUA | UACCAUUUCCACCUCCACC | | [3039-3057] ORF |
| 383 | GCAAUGGCUACCUGUCCUU | AAGGACAGGUAGCCAUUGC | | [2707-2725] ORF |
| 384 | AGGAAAGAAGACAUCUGAA | UUCAGAUGUCUUCUUUCCU | | [148-166] 5′UTR |
| 385 | CUGGAGUGGUGGCCUAUUU | AAAUAGGCCACCACUCCAG | | [2050-2068] ORF |
| 386 | AGAGGAAAGAAGACAUCUG | CAGAUGUCUUCUUUCCUCU | | [146-164] 5′UTR |
| 387 | GGAACUGGAGUGAUCUCAA | UUGAGAUCACUCCAGUUCC | | [1564-1582] ORF |
| 388 | GGAGUUGGAGGACAACAUA | UAUGUUGUCCUCCAACUCC | | [1400-1418] ORF |
| 389 | CCAGUGCAGAAGCUGUGAU | AUCACAGCUUCUGCACUGG | | [5403-5421] 3′UTR |
| 390 | CCAGUCAGGAUCUCCCAAA | UUUGGGAGAUCCUGACUGG | | [5140-5158] 3′UTR |
| 391 | GCACAUUUAUGUCACCCAG | CUGGGUGACAUAAAUGUGC | | [4544-4562] ORF |
| 392 | GGCAGCCAAAUGCUGUGUA | UACACAGCAUUUGGCUGCC | | [4419-4437] ORF |
| 393 | GAGUACAAGUCAGGACAGU | ACUGUCCUGACUUGUACUC | | [4089-4107] ORF |
| 394 | CCCAAGGCUUUGAGUACAA | UUGUACUCAAAGCCUUGGG | | [4078-4096] ORF |
| 395 | CGCAGUCAAUGUCUACAUC | GAUGUAGACAUUGACUGCG | | [3638-3658] ORF |
| 396 | ACGCAGUCAAUGUCUACAU | AUGUAGACAUUGACUGCGU | | [3637-3655] ORF |
| 397 | AGGUGGAGGUGGAAAUGGU | ACCAUUUCCACCUCCACCU | | [3038-3056] ORF |
| 398 | UGCCUUCCCUUAGUGAGUC | GACUCACUAAGGGAAGGCA | | [2022-2040] ORF |
| 399 | CCAGUUUUGGUCCUACUAA | UUAGUAGGACCAAAACUGG | | [6114-6132] 3′UTR |
| 400 | AUCGGGCUCUUUCCUAAAA | UUUUAGGAAAGAGCCCGAU | | [5911-5929] 3′UTR |
| 401 | CUCUGCCAGAUAUUCAGUA | UACUGAAUAUCUGGCAGAG | | [5685-5703] 3′UTR |
| 402 | AAAAGGAGCAGCCCUACUU | AAGUAGGGCUGCUCCUUUU | | [5484-5502] 3′UTR |
| 403 | GGAGUAUACAGGAUACCGU | ACGGUAUCCUGUAUACUCC | | [1136-1154] ORF |
| 404 | GCAGUCAAUGUCUACAUCU | AGAUGUAGACAUUGACUGC | | [3639-3657] ORF |
| 405 | GCAGCUCUGUGUCAAAGGU | ACCUUUGACACAGAGCUGC | | [3017-3035] ORF |
| 406 | CUUCUUCAGACACCUUUUU | AAAAAGGUGUCUGAAGAAG | | [2513-2531] ORF |
| 407 | CUGCUGCUCAAGAUCCCUA | UAGGGAUCUUGAGCAGCAG | | [2319-2337] ORF |
| 408 | GGUCGCUGUUAUCAACAUU | AAUGUUGAUAACAGCGACC | | [1835-1853] ORF |
| 409 | UCCGGAAGGUCCUGAACAA | UUGUUCAGGACCUUCCGGA | | [1261-1279] ORF |
| 410 | GGUGAAGGCAAGAAGUAGA | UCUACUUCUUGCCUUCACC | | [107-125] 5′UTR |
| 411 | CAAAUCCAGUUUUGGUCCU | AGGACCAAAACUGGAUUUG | | [6109-6127] 3′UTR |
| 412 | UGAUGUGAUUUCUACCAAA | UUUGGUAGAAAUCACAUCA | | [6094-6112] 3′UTR |
| 413 | GUACAUGUUUUACCACCAC | GUGGUGGUAAAACAUGUAC | | [5976-5994] 3′UTR |
| 414 | UCAGCUCUUUAACUUUUCU | AGAAAAGUUAAAGAGCUGA | | [5881-5899] 3′UTR |
| 415 | GGAUCUCCCAAAGAAGAUA | UAUCUUCUUUGGGAGAUCC | | [5147-5165] 3′UTR |
| 416 | CCGGGAGUAUACAGGAUAC | GUAUCCUGUAUACUCCGGG | | [1133-1151] ORF |
| 417 | CAAUUGCAGUGCAUGAUGA | UCAUCAUGCACUGCAAUUG | | [5047-5065] 3′UTR |
| 418 | UACAAUUGCAGUGCAUGAU | AUCAUGCACUGCAAUUGUA | | [5045-5063] 3′UTR |
| 419 | UUCUACAAUUGCAGUGCAU | AUGCACUGCAAUUGUAGAA | | [5042-5060] 3′UTR |
| 420 | AGAAAACACUCCCGGAGUA | UACUCCGGGAGUGUUUUCU | | [1123-1141] ORF |
| 421 | ACCAGAACAUCGCUGUGUA | UACACAGCGAUGUUCUGGU | | [1081-1099] ORF |
| 422 | CCGCUAUGUGCCUUUUGAU | AUCAAAAGGCACAUAGCGG | | [3548-3568] ORF |
| 423 | CCAAAAGCUGCAGCAGUAC | GUACUGCUGCAGCUUUUGG | | [3278-3296] ORF |
| 424 | GAGCUCUUCUGACUGGAUC | GAUCCAGUCAGAAGAGCUC | | [241-259] ORF |
| 425 | CUCAGGUGCUGGACAUCAA | UUGAUGUCCAGCACCUGAG | Rat, Mouse | [2533-2551] ORF |
| 426 | UGAGCGAGAAGGAGCUAUU | AAUAGCUCCUUCUCGCUCA | | [2449-2467] ORF |
| 427 | CCAUCACCAUCAUUGCUCU | AGAGCAAUGAUGGUGAUGG | | [1999-2017] ORF |
| 428 | ACGUGCUGGUCGCUGUUAU | AUAACAGCGACCAGCACGU | | [1828-1846] ORF |
| 429 | CGCUACUGGUUUGAGAACA | UGUUCUCAAACCAGUAGCG | Rat, Mouse | [1749-1767] ORF |
| 430 | AGGACAACAUAGUGGUUGA | UCAACCACUAUGUUGUCCU | | [1406-1426] ORF |
| 431 | GUGAAGGCAAGAAGUAGAG | CUCUACUUCUUGCCUUCAC | | [106-128] 5′UTR |
| 432 | AGGUCCUGAACAAGGGUUU | AAACCCUUGUUCAGGACCU | | [1267-1285] ORF |
| 433 | AAAUCCAGUUUUGGUCCUA | UAGGACCAAAACUGGAUUU | | [6110-6128] 3′UTR |
| 434 | CGGGCUCUUUCCUAAAAAG | CUUUUUAGGAAAGAGCCCG | | [5913-5931] 3′UTR |
| 435 | CAGUCAGGAUCUCCCAAAG | CUUUGGGAGAUCCUGACUG | | [5141-5159] 3′UTR |
| 436 | CCAAUAUUGGGAGAAUAUU | AAUAUUCUCCCAAUAUUGG | | [4981-4999] 3′UTR |
| 437 | CCCAAUAUGGGGAGAAUAU | AUAUUCUCCCAAUAUUGGG | | [4980-4998] 3′UTR |
| 438 | GCUUCCAGUAUCCUGCCUU | AAGGCAGGAUACUGGAAGC | | [4871-4889] 3′UTR |
| 439 | CCACCAUGCUAUACAUCUG | CAGAUGUAUAGCAUGGUGG | | [4588-4606] ORF |
| 440 | GCAGCCAAAUGCUGUGUAA | UUACACAGCAUUUGGCUGC | | [4420-4438] ORF |
| 441 | UCAACCGCUAUGUGCCUUU | AAAGGCACAUAGCGGUUGA | | [3544-3582] ORF |
| 442 | GCGUCUCCUUCAUGUUCUC | GAGAACAUGAAGGAGACGC | | [3469-3487] ORF |
| 443 | CUGGACUGAAGAAGAGGUU | AACCUCUUCUUCAGUCCAG | | [3181-3199] ORF |
| 444 | AGGUGGUGGAGUCUAUGUU | AACAUAGACUCCACCACCU | | [2908-2926] ORF |
| 445 | GGAUGAGAAUGGCUUCCUC | GAGGAAGCCAUUCUCAUCC | | [2810-2828] ORF |
| 446 | UACCAUGUAUGACCUGGAU | AUCCAGGUCAUACAUGGUA | Rat, Mouse | [2795-2813] ORF |
| 447 | UGCAGCAGGUCAACCUCAU | AUGAGGUUGACCUGCUGCA | | [2272-2290] ORF |
| 448 | AGCUACAAAAGAAACUCAA | UUGAGUUUCUUUUGUAGCU | | [2089-2107] ORF |
| 449 | GAGUCUGCUUCUCUCUGGA | UCCAGAGAGAAGCAGACUC | | [2036-2054] ORF |
| 450 | CCUCAACCUAAGCAGCUCA | UGAGCUGCUUAGGUUGAGG | | [1906-1926] ORF |
| 451 | GGUUUGAGAACACCAGGAA | UUCCUGGUGUUCUCAAACC | | [1756-1774] ORF |
| 452 | CCUGGCAAAUUCUCCCGUA | UACGGGAGAAUUUGCCAGG | | [1452-1470] ORF |
| 453 | GGGUUUUCAAAGCUCCCAA | UUGGGAGCUUUGAAAACCC | | [1280-1298] ORF |
| 454 | GAUGUGAUUUCUACCAAAU | AUUUGGUAGAAAUCACAUC | | [6095-6113] 3′UTR |
| 455 | CCCAGGUCAAGACAUAGAA | UUCUAUGUCUUGACCUGGG | | [5994-6012] 3′UTR |
| 456 | CAGCUCUUUAACUUUUCUU | AAGAAAAGUUAAAGAGCUG | | [5882-5900] 3′UTR |
| 457 | CUCAGCUCUUUAACUUUUC | GAAAAGUUAAAGAGCUGAG | | [5880-5898] 3′UTR |
| 458 | ACUCAGCUCUUUAACUUUU | AAAAGUUAAAGAGCUGAGU | | [5879-5897] 3′UTR |
| 459 | GAAGCUGUGAUGCUUAGAA | UUCUAAGCAUCACAGCUUC | | [5411-5429] 3′UTR |
| 460 | CCCAGCUUCCUGCAGAAAA | UUUUCUGCAGGAAGCUGGG | Rat, Mouse | [1110-1128] ORF |
| 461 | CUCAGGACCACCAUGCUAU | AUAGCAUGGUGGUCCUGAG | | [4581-4599] ORF |
| 462 | UGGUGUCUGUGCACAUUUA | UAAAUGUGCACAGACACCA | | [4534-4552] ORF |
| 463 | CCAGAACAUCGCUGUGUAU | AUACACAGCGAUGUUCUGG | | [1082-1100] ORF |
| 464 | UCAGGAGUGGCAUAAAUUU | AAAUUUAUGCCACUCCUGA | | [4316-4334] ORF |
| 465 | CCACAUCUACUUCCUGGUC | GACCAGGAAGUAGAUGUGG | Rat, Mouse | [3941-3959] ORF |
| 466 | GCAGCUAUGCUCUGAUCCA | UGGAUCAGAGCAUAGCUGC | | [3910-3928] ORF |
| 467 | ACCGCUAUGUGCCUUUUGA | UCAAAAGGCACAUAGCGGU | | [3547-3565] ORF |
| 468 | GAGGCUUCCUAGCCCAAAA | UUUUGGGCUAGGAAGCCUC | | [3265-3283] ORF |
| 469 | ACAAGGAGGAGCUGACAUG | CAUGUCAGCUCCUCCUUGU | | [2947-2965] ORF |
| 470 | CCAACAACUGCCUGUCCAA | UUGGACAGGCAGUUGUUGG | Rat, Mouse | [2875-2893] ORF |
| 471 | AGAAUGGCUUCCUCUCCAA | UUGGAGAGGAAGCCAUUCU | | [2815-2833] ORF |
| 472 | CCUGGUGGUCUUCAUGAAA | UUUCAUGAAGACCACCAGG | Mouse | [2744-2762] ORF |
| 473 | UGCUCAAGAUCCCUAAGGA | UCCUUAGGGAUCUUGAGCA | | [2323-2341] ORF |
| 474 | CUGUCCAACAACCGAGGAU | AUCCUCGGUUGUUGGACAG | | [2292-2310] ORF |
| 475 | CAACCUAAGCAGCUCACAA | UUGUGAGCUGCUUAGGUUG | | [1911-1929] ORF |
| 476 | CUCAACCUAAGCAGCUCAC | GUGAGCUGCUUAGGUUGAG | | [1909-1927] ORF |
| 477 | AGAGCACAGAAGCAAAGAU | AUCUUUGCUUCUGUGCUCU | | [123-141] 5′UTR |
| 478 | ACAACAUAGUGGUUGAAGA | UCUUCAACCACUAUGUUGU | | [1411-1429] ORF |
| 479 | GAGGACAACAUAGUGGUUG | CAACCACUAUGUUGUCCUC | | [1407-1425] ORF |
| 480 | GUCCUGAACAAGGGUUUUC | GAAAACCCUUGUUCAGGAC | | [1269-1287] ORF |
| 481 | GGUCCUGAACAAGGGUUUU | AAAACCCUUGUUCAGGACC | | [1268-1286] ORF |
| 482 | GUCUACAUGAGAAAUGCCA | UGGCAUUUCUCAUGUAGAC | | [1233-1251] ORF |
| 483 | GCACCAGGAAACAAAGAUG | CAUCUUUGUUUCCUGGUGC | | [5545-5563] 3′UTR |
| 484 | CAGAAGCUGUGAUGCUUAG | CUAAGCAUCACAGCUUCUG | | [5409-5427] 3′UTR |
| 485 | GCAGAAGCUGUGAUGCUUA | UAAGCAUCACAGCUUCUGC | | [5408-5426] 3′UTR |
| 486 | CCGGAGUAUACAGGAUACC | GGUAUCCUGUAUACUCCGG | | [1134-1152] ORF |
| 487 | UCCCGGAGUAUACAGGAUA | UAUCCUGUAUACUCCGGGA | | [1132-1150] ORF |
| 488 | GUUCUACAAUUGCAGUGCA | UGCACUGCAAUUGUAGAAC | | [5041-5059] 3′UTR |
| 489 | UCCCAAUAUUGGGAGAAUA | UAUUCUCCCAAUAUUGGGA | | [4979-4997] 3′UTR |
| 490 | CUCCAGGAAUGACCAAGAA | UUCUUGGUCAUUCCUGGAG | | [4759-4777] ORF |
| 491 | UGCACAUUUAUGUCACCCA | UGGGUGACAUAAAUGUGCA | | [4543-4561] ORF |
| 492 | AGGAGUGGCAUAAAUUUGA | UCAAAUUUAUGCCACUCCU | | [4318-4336] ORF |
| 493 | CCACGCAGUCAAUGUCUAC | GUAGACAUUGACUGCGUGG | | [3635-3653] ORF |
| 494 | GCCAUUUUGCACAGUGCUG | CAGCACUGUGCAAAAUGGC | | [3615-3633] ORF |
| 495 | UGCGAGAGACUUUCCUCAA | UUGAGGAAAGUCUCUCGCA | | [3529-3547] ORF |
| 496 | CCCAAAAGCUGCAGCAGUA | UACUGCUGCAGCUUUUGGG | | [3277-3295] ORF |
| 497 | GCUGGUUUAACAACCUGAG | CUCAGGUUGUUAAACCAGC | | [322-340] ORF |
| 498 | UGCGAUCCUUCAUCGAGAU | AUCUCGAUGAAGGAUCGCA | | [2854-2872] ORF |
| 499 | AGGACGAAUUCUUCACCAU | AUGGUGAAGAAUUCGUCCU | | [2833-2851] ORF |
| 500 | AGAGGAUAAGUCCCGUCUA | UAGACGGGACUUAUCCUCU | | [2771-2789] ORF |

TABLE J
21-mer oligomers for DUOX2

| | | | | |
|---|---|---|---|---|
| | | | | Human-132566531 |
| Number | Sense siRNA | AntiSense siRNA | Other Sp | ORF: 204-4850 |

| | | | | |
|---|---|---|---|---|
| 1 | CCAGACAAUUAUACAAAUGAG | CUCAUUUGUAUAAUUGUCUGG | | [5003-5023] 3′UTR |
| 2 | UGAACCCAACACCACCCUAAA | UUUAGGGUGGUGUUGGGUUCA | | [163-183] 5′UTR |
| 3 | CCCAGACAAUUAUACAAAUGA | UCAUUUGUAUAAUUGUCUGGG | | [5002-5022] 3′UTR |
| 4 | GAGAAUAUUGACCCAGACAAU | AUUGUCUGGGUCAAUAUUCUC | | [4991-5011] 3′UTR |
| 5 | CCGUCUAAUGUUUACCAUGUA | UACAUGGUAAACAUUAGACGG | | [2783-2803] ORF |
| 6 | ACCCAGACAAUUAUACAAAUG | CAUUUGUAUAAUUGUCUGGGU | | [5001-5021] 3′UTR |
| 7 | CCAAAUGCUGUGUAAGAAGAU | AUCUUCUUACACAGCAUUUGG | | [4424-4444] ORF |
| 8 | CGUCUAAUGUUUACCAUGUAU | AUACAUGGUAAACAUUAGACG | | [2784-2804] ORF |
| 9 | ACCCAACACCACCCUAAACCA | UGGUUUAGGGUGGUGUUGGGU | | [166-186] 5′UTR |
| 10 | CCCUGGUGUCUACAUGAGAAA | UUUCUCAUGUAGACACCAGGG | | [1226-1246] ORF |
| 11 | UCAGUACUCGAUCUCAAUUCC | GGAAUUGAGAUCGAGUACUGA | | [5698-5718] 3′UTR |
| 12 | UCUCUAAUUGACUUCCACAAA | UUUGUGGAAGUCAAUUAGAGA | | [5298-5316] 3′UTR |
| 13 | GGAAAUGGUAUUAGAGAUAUC | GAUAUCUCUAAUACCAUUUCC | | [3048-3068] ORF |
| 14 | GGAGCUAUUUAGGAAGGCUGU | ACAGCCUUCCUAAAUAGCUCC | | [2459-2479] ORF |
| 15 | GCACAGAAGCAAAGAUUUUAA | UUAAAAUCUUUGCUUCUGUGC | | [126-146] 5′UTR |
| 16 | CUCUAAUUGACUUCCACAAAA | UUUUGUGGAAGUCAAUUAGAG | | [5297-5317] 3′UTR |
| 17 | CGAGAAGGAGCUAUUUAGGAA | UUCCUAAAUAGCUCCUUCUCG | | [2453-2473] ORF |
| 18 | GGUCAACCUCAUCCUGUCCAA | UUGGACAGGAUGAGGUUGACC | | [2279-2299] ORF |
| 19 | ACAAAAUCGAUGCGUUGCUUU | AAAGCAACGCAUCGAUUUUGU | | [5312-5332] 3′UTR |
| 20 | GCCCACUUUCCACAUCUACUU | AAGUAGAUGUGGAAAGUGGGC | | [3932-3952] ORF |
| 21 | CCCAACGUCUUUGUGAAUGAU | AUCAUUCACAAAGACGUUGGG | | [3698-3716] ORF |
| 22 | CUCCUUCAUGUUCUCUUAUAU | AUAUAAGAGAACAUGAAGGAG | | [3473-3493] ORF |
| 23 | GGAGCUGACAUGGGAGGAUUU | AAAUCCUCCCAUGUCAGCUCC | | [2954-2974] ORF |
| 24 | CCAACACCACCCUAAACCACA | UGUGGUUUAGGGUGGUGUUGG | | [168-188] 5′UTR |
| 25 | CUAUUUAGGAAGGCUGUGACA | UGUCACAGCCUUCCUAAAUAG | | [2463-2483] ORF |
| 26 | GCUAUUUAGGAAGGCUGUGAC | GUCACAGCCUUCCUAAAUAGC | | [2462-2482] ORF |
| 27 | CAACCUCAUCCUGUCCAACAA | UUGUUGGACAGGAUGAGGUUG | | [2282-2302] ORF |
| 28 | AGAAGACAUCUGAACCCAACA | UGUUGGGUUCAGAUGUCUUCU | | [153-173] 5′UTR |
| 29 | CGAGAACACAAGAAGCUACAA | UUGUAGCUUCUUGUGUUCUCG | | [2078-2096] ORF |
| 30 | CAACUACUGGAUUCGGGAGAA | UUCUCCCGAAUCCAGUAGUUG | Rat | [1316-1338] ORF |
| 31 | ACAUCUCUAAUUGACUUCCAC | GUGGAAGUCAAUUAGAGAUGU | | [5293-5313] 3′UTR |
| 32 | CCCACAUCUCUAAUUGACUUC | GAAGUCAAUUAGAGAUGUGGG | | [5290-5310] 3′UTR |
| 33 | GGAGGUGAAAAGGUACAAUAG | CUAUUGUACCUUUUCACCUCC | | [5250-5270] 3′UTR |
| 34 | GGAGACUAUGUUCUACAAUUG | CAAUUGUAGAACAUAGUCUCC | | [5032-5052] 3′UTR |
| 35 | CCCACUUUCCACAUCUACUUC | GAAGUAGAUGUGGAAAGUGGG | | [3933-3953] ORF |
| 36 | CUCUUAUAUCUUGCUCACCAU | AUGGUGAGCAAGAUAUAAGAG | | [3485-3505] ORF |
| 37 | GUGGAAAUGGUAUUAGAGAUA | UAUCUCUAAUACCAUUUCCAC | | [3046-3068] ORF |
| 38 | GGAGGUGGAAAUGGUAUUAGA | UCUAAUACCAUUUCCACCUCC | | [3042-3062] ORF |
| 39 | CACAAGAAGCUACAAAAGAAA | UUUCUUUUGUAGCUUCUUGUG | | [2082-2102] ORF |
| 40 | AGAACACAAGAAGCUACAAAA | UUUUGUAGCUUCUUGUGUUCU | | [2078-2098] ORF |
| 41 | GCCAUUGUCCUCGACCAGUUU | AAACUGGUCGAGGACAAUGGC | | [1707-1727] ORF |
| 42 | UGCCAGAUAUUCAGUACUCGA | UCGAGUACUGAAUAUCUGGCA | | [5688-5708] 3′UTR |
| 43 | GCAGUGCAUGAUGAUUAUAAG | CUUAUAAUCAUCAUGCACUGC | | [5052-5072] 3′UTR |
| 44 | GGAAUGACCAAGAAUGUAGAG | CUCUACAUUCUUGGUCAUUCC | | [4764-4784] ORF |
| 45 | GCUGUUGUCCUGGCCAUUUUG | CAAAAUGGCCAGGACAACAGC | | [3603-3623] ORF |
| 46 | UGCUGUUGUCCUGGCCAUUUU | AAAAUGGCCAGGACAACAGCA | | [3602-3622] ORF |
| 47 | CCGCAACCUCAUAACCUUCCU | AGGAAGGUUAUGAGGUUGCGG | | [3509-3529] ORF |
| 48 | GUGGAGGUGGAAAUGGUAUUA | UAAUACCAUUUCCACCUCCAC | | [3040-3080] ORF |
| 49 | AAAGAAGACAUCUGAACCCAA | UUGGGUUCAGAUGUCUUCUUU | | [151-171] 5′UTR |
| 50 | GGUCUGCAACAACUACUGGAU | AUCCAGUAGUUGUUGCAGACC | Rat | [1307-1327] ORF |
| 51 | CCUGGUGUCUACAUGAGAAAU | AUUUCUCAUGUAGACACCAGG | | [1227-1247] ORF |
| 52 | CAGUUUUGGUCCUACUAAAUA | UAUUUAGUAGGACCAAAACUG | | [6115-6135] 3′UTR |
| 53 | CCCUCCAUACUCAGCUCUUUA | UAAAGAGCUGAGUAUGGAGGG | | [5871-5891] 3′UTR |
| 54 | CACAUCUCUAAUUGACUUCCA | UGGAAGUCAAUUAGAGAUGUG | | [5292-5312] 3′UTR |
| 55 | UGGAGGUGAAAAGGUACAAUA | UAUUGUACCUUUUCACCUCCA | | [5249-5269] 3′UTR |
| 56 | GUGACAAGUGUGGAGGUGAAA | UUUCACCUCCACACUUGUCAC | | [5239-5259] 3′UTR |
| 57 | CCCAAAGAAGAUAAAGACCAC | GUGGUCUUUAUCUUCUUUGGG | | [5153-5173] 3′UTR |
| 58 | UGACCCAGACAAUUAUACAAA | UUUGUAUAAUUGUCUGGGUCA | | [4999-5019] 3′UTR |
| 59 | CCAAGAAUGUAGAGAAGGCCU | AGGCCUUCUCUACAUUCUUGG | | [4771-4791] ORF |
| 60 | GGCAAUCAUCUAUGGAGGUGA | UCACCUCCAUAGAUGAUUGCC | | [3962-3982] ORF |
| 61 | GCUGCCCACUUUCCACAUCUA | UAGAUGUGGAAAGUGGGCAGC | | [3929-3949] ORF |
| 62 | CCACCUCUACAUCCUGCUCUA | UAGAGCAGGAUGUAGAGGUGG | | [3889-3889] ORF |
| 63 | CCUGGCCAUCAUGUAUGUCUU | AAGACAUACAUGAUGGCCAGG | | [3797-3817] ORF |
| 64 | GAAGAAGAGGUUUGGCAAAAA | UUUUUGCCAAACCUCUUCUUC | | [3188-3208] ORF |
| 65 | GAAAGAAGACAUCUGAACCCA | UGGGUUCAGAUGUCUUCUUUC | | [150-170] 5′UTR |
| 66 | AGAAGGAAGCAGCCAAAGAUG | CAUCUUUGGCUGCUUCCUUCU | | [2119-2139] ORF |
| 67 | GUGAAGAAGGAAGCAGCCAAA | UUUGGCUGCUUCCUUCUUCAC | | [2115-2135] ORF |
| 68 | GCUACAAAAGAAACUCAAAGA | UCUUUGAGUUUCUUUUGUAGC | | [2090-2110] ORF |
| 69 | ACACAAGAAGCUACAAAAGAA | UUCUUUUGUAGCUUCUUGUGU | | [2081-2101] ORF |
| 70 | CCAAUGUCUUUGUCUGGCAUA | UAUGCCAGACAAAGACAUUGG | | [1873-1893] ORF |
| 71 | CAACAACUACUGGAUUCGGGA | UCCCGAAUCCAGUAGUUGUUG | Rat | [1313-1333] ORF |
| 72 | UCUCAGGGUCUGCAACAACUA | UAGUUGUUGCAGACCCUGAGA | Rat | [1301-1321] ORF |
| 73 | GCUCUUUAACUUUUCUUUUCC | GGAAAAGAAAAGUUAAAGAGC | | [5884-5904] 3′UTR |
| 74 | CCAAAGAAGAUAAAGACCACU | AGUGGUCUUUAUCUUCUUUGG | | [5154-5174] 3′UTR |
| 75 | GACCCAGACAAUUAUACAAAU | AUUUGUAUAAUUGUCUGGGUC | | [5000-5020] 3′UTR |
| 76 | CUCAUCAUCCAUGGCAGCUAU | AUAGCUGCCAUGGAUGAUGAG | | [3897-3917] ORF |
| 77 | GAAGUUCUAUUGGUGGUUCUU | AAGAACCACCAAUAGAACUUC | | [3734-3754] ORF |
| 78 | CCAACGUCUUUGUGAAUGAUG | CAUCAUUCACAAAGACGUUGG | | [3697-3717] ORF |
| 79 | GGAGGAUUUUCACUUCAUGCU | AGCAUGAAGUGAAAAUCCUCC | | [2966-2986] ORF |
| 80 | GUCUAAUGUUUACCAUGUAUG | CAUACAUGGUAAACAUUAGAC | | [2785-2805] ORF |
| 81 | AGGAGCUAUUUAGGAAGGCUG | CAGCCUUCCUAAAUAGCUCCU | | [2458-2478] ORF |
| 82 | GAGAAGGAGCUAUUUAGGAAG | CUUCCUAAAUAGCUCCUUCUC | | [2454-2474] ORF |
| 83 | CUACAAAAGAAACUCAAAGAG | CUCUUUGAGUUUCUUUUGUAG | | [2091-2111] ORF |
| 84 | GAACACAAGAAGCUACAAAAG | CUUUUGUAGCUUCUUGUGUUC | | [2079-2099] ORF |
| 85 | GUCUGCAACAACUACUGGAUU | AAUCCAGUAGUUGUUGCAGAC | Rat | [1308-1328] ORF |
| 86 | CCAGGUCAAGACAUAGAAUGU | ACAUUCUAUGUCUUGACCUGG | | [5995-6015] 3′UTR |
| 87 | CCAGAUAUUCAGUACUCGAUC | GAUCGAGUACUGAAUAUCUGG | | [5690-5710] 3′UTR |
| 88 | CCCAGCCCUGAUAUUCUUGAU | AUCAAGAAUAUCAGGGCUGGG | | [5568-6588] 3′UTR |
| 89 | CCACAUCUCUAAUUGACUUCC | GGAAGUCAAUUAGAGAUGUGG | | [5291-5311] 3′UTR |
| 90 | GUGGAGGUGAAAAGGUACAAU | AUUGUACCUUUUCACCUCCAC | | [5248-5268] 3′UTR |
| 91 | UGAUUAUAAGUCCACCUGUUU | AAACAGGUGGACUUAUAAUCA | | [5063-5083] 3′UTR |
| 92 | GCAUGAUGAUUAUAAGUCCAC | GUGGACUUAUAAUCAUCAUGC | | [5057-5077] 3′UTR |
| 93 | ACAAUUGCAGUGCAUGAUGAU | AUCAUCAUGCACUGCAAUUGU | | [5046-5088] 3′UTR |
| 94 | CAGGAGACUAUGUUCUACAAU | AUUGUAGAACAUAGUCUCCUG | | [5030-5050] 3′UTR |
| 95 | GGCAUAAAUUUGAGGUGUCAG | CUGACACCUCAAAUUUAUGCC | | [4324-4344] ORF |
| 96 | GGUCCUGGCCAUCAUGUAUGU | ACAUACAUGAUGGCCAGGACC | | [3794-3814] ORF |
| 97 | CGCAACCUCAUAACCUUCCUG | CAGGAAGGUUAUGAGGUUGCG | | [3510-3530] ORF |
| 98 | UCCUUCAUGUUCUCUUAUAUC | GAUAUAAGAGAACAUGAAGGA | | [3474-3494] ORF |
| 99 | CGUGCUUACUACUAUGGCUUU | AAAGCCAUAGUAGUAAGCACG | | [3378-3398] ORF |
| 100 | GAGGAUUUUCACUUCAUGCUG | CAGCAUGAAGUGAAAAUCCUC | | [2967-2987] ORF |
| 101 | CCCGUCUAAUGUUUACCAUGU | ACAUGGUAAACAUUAGACGGG | | [2782-2802] ORF |
| 102 | CCGAGAACACAAGAAGCUACA | UGUAGCUUCUUGUGUUCUCGG | | [2075-2095] ORF |
| 103 | GGCCGAGAACACAAGAAGCUA | UAGCUUCUUGUGUUCUCGGCC | | [2073-2093] ORF |
| 104 | GAAGCAAAGAUUUUAAGAGGA | UCCUCUUAAAAUCUUUGCUUC | | [131-151] 5′UTR |
| 105 | GGCAAGAAGUAGAGCACAGAA | UUCUGUGCUCUACUUCUUGCC | | [113-133] 5′UTR |
| 106 | AGAUAUUCAGUACUCGAUCUC | GAGAUCGAGUACUGAAUAUCU | | [5692-5712] 3′UTR |
| 107 | CGAUGCGUUGCUUUGGUAUUU | AAAUACCAAAGCAACGCAUCG | | [5319-5339] 3′UTR |
| 108 | GCAUAAAUUUGAGGUGUCAGU | ACUGACACCUCAAAUUUAUGC | | [4325-4345] ORF |
| 109 | GGCUUUGAGUACAAGUCAGGA | UCCUGACUUGUACUCAAAGCC | | [4083-4103] ORF |
| 110 | CACUUUCCACAUCUACUUCCU | AGGAAGUAGAUGUGGAAAGUG | | [3935-3955] ORF |
| 111 | GCUCUAUGCCCUGCUCAUCAU | AUGAUGAGCAGGGCAUAGAGC | | [3884-3904] ORF |
| 112 | CCAUCAUGUAUGUCUUCGCCU | AGGCGAAGACAUACAUGAUGG | | [3802-3822] ORF |
| 113 | UGUCUACAUCUUCUCAGUCAG | CUGACUGAGAAGAUGUAGACA | | [3647-3667] ORF |
| 114 | CUGCUGUUGUCCUGGCCAUUU | AAAUGGCCAGGACAACAGCAG | | [3601-3621] ORF |
| 115 | GUCUCCUUCAUGUUCUCUUAU | AUAAGAGAACAUGAAGGAGAC | | [3471-3491] ORF |
| 116 | UCGUGCUUACUACUAUGGCUU | AAGCCAUAGUAGUAAGCACGA | | [3377-3397] ORF |
| 117 | CCAUCUGUGUUGGCGUGUUUG | CAAACACGCCAACACAGAUGG | | [3352-3372] ORF |
| 118 | GGUGGAAAUGGUAUUAGAGAU | AUCUCUAAUACCAUUUCCACC | | [3045-3065] ORF |
| 119 | CCAGAGAGAAAGGUGAGAGUG | CACUCUCACCUUUCUCUCUGG | | [45-65] 5′UTR |
| 120 | GGGAGGAUUUUCACUUCAUGC | GCAUGAAGUGAAAAUCCUCCC | | [2965-2985] ORF |
| 121 | UGGGAGGAUUUUCACUUCAUG | CAUGAAGUGAAAAUCCUCCCA | | [2964-2984] ORF |
| 122 | CUAUGACGGCUGGUUUAACAA | UUGUUAAACCAGCCGUCAUAG | | [314-334] ORF |
| 123 | GUCCCGUCUAAUGUUUACCAU | AUGGUAAACAUUAGACGGGAC | | [2780-2800] ORF |
| 124 | GCGAGAAGGAGCUAUUUAGGA | UCCUAAAUAGCUCCUUCUCGC | | [2452-2472] ORF |
| 125 | GCUGUUUAGUUCUGAAGAGGA | UCCUCUUCAGAACUAAACAGC | | [2357-2377] ORF |
| 126 | UGAAGAAGGAAGCAGCCAAAG | CUUUGGCUGCUUCCUUCUUCA | | [2116-2138] ORF |
| 127 | ACAAGAAGCUACAAAAGAAAC | GUUUCUUUUGUAGCUUCUUGU | | [2083-2103] ORF |
| 128 | CUGACUGUGCUUGACUUCUUU | AAAGAAGUCAAGCACAGUCAG | | [1956-1976] ORF |
| 129 | UGAACAAGGGUUUUCAAAGCU | AGCUUUGAAAACCCUUGUUCA | | [1273-1293] ORF |
| 130 | CCUAAAAAGCUGAGCUGUAAA | UUUACAGCUCAGCUUUUUAGG | | [5923-5943] 3′UTR |
| 131 | GCCCUGAUAUUCUUGAUGUCA | UGACAUCAAGAAUAUCAGGGC | | [5572-5592] 3′UTR |
| 132 | GGACUGACAUUUGAGGGAGGA | UCCUCCCUCAAAUGUCAGUCC | | [5344-5384] 3′UTR |
| 133 | UGACAAGUGUGGAGGUGAAAA | UUUUCACCUCCACACUUGUCA | | [5240-5260] 3′UTR |
| 134 | CCUUCUCUUCUGUGCACCUAA | UUAGGUGCACAGAAGAGAAGG | | [4886-4906] 3′UTR |
| 135 | AGAACAUCGCUGUGUAUGAGU | ACUCAUACACAGCGAUGUUCU | | [1084-1104] ORF |
| 136 | GCCAAAUGCUGUGUAAGAAGA | UCUUCUUACACAGCAUUUGGC | | [4423-4443] ORF |
| 137 | UGGCAUAAAUUUGAGGUGUCA | UGACACCUCAAAUUUAUGCCA | | [4323-4343] ORF |
| 138 | CAAGGCUUUGAGUACAAGUCA | UGACUUGUACUCAAAGCCUUG | | [4080-4100] ORF |
| 139 | CUGCCCACUUUCCACAUCUAC | GUAGAUGUGGAAAGUGGGCAG | | [3930-3950] ORF |
| 140 | CUCUAUGCCCUGCUCAUCAUC | GAUGAUGAGCAGGGCAUAGAG | | [3885-3905] ORF |
| 141 | CACCUCUACAUCCUGCUCUAU | AUAGAGCAGGAUGUAGAGGUG | | [3870-3890] ORF |
| 142 | AGAAGUUCUAUUGGUGGUUCU | AGAACCACCAAUAGAACUUCU | | [3733-3753] ORF |
| 143 | CCUUCAUGUUCUCUUAUAUCU | AGAUAUAAGAGAACAUGAAGG | | [3475-3495] ORF |
| 144 | GAGGUGGAAAUGGUAUUAGAG | CUCUAAUACCAUUUCCACCUC | | [3043-3063] ORF |
| 145 | AUGGGAGGAUUUUCACUUCAU | AUGAAGUGAAAAUCCUCCCAU | | [2963-2983] ORF |
| 146 | ACAUGGGAGGAUUUUCACUUC | GAAGUGAAAAUCCUCCCAUGU | | [2961-2981] ORF |
| 147 | CUGACAUGGGAGGAUUUUCAC | GUGAAAAUCCUCCCAUGUCAG | | [2958-2978] ORF |
| 148 | AUGUAUGACCUGGAUGAGAAU | AUUCUCAUCCAGGUCAUACAU | | [2799-2819] ORF |
| 149 | AUAAGUCCCGUCUAAUGUUUA | UAAACAUUAGACGGGACUUAU | | [2776-2798] ORF |
| 150 | CUGUUUAGUUCUGAAGAGGAA | UUCCUCUUCAGAACUAAACAG | | [2358-2378] ORF |
| 151 | GAGCAGUCCCAUCAUCAUCCA | UGGAUGAUGAUGGGACUGCUC | | [2177-2197] ORF |
| 152 | CGUGAAGAAGGAAGCAGCCAA | UUGGCUGCUUCCUUCUUCACG | | [2114-2134] ORF |
| 153 | GAGAACACAAGAAGCUACAAA | UUUGUAGCUUCUUGUGUUCUC | | [2077-2097] ORF |
| 154 | AGACAUCCGAAAUACCACCCU | AGGGUGGUAUUUCGGAUGUCU | | [1802-1822] ORF |
| 155 | AGGUCAAGACAUAGAAUGUUU | AAACAUUCUAUGUCUUGACCU | | [5997-6017] 3′UTR |
| 156 | UCAUCGGGCUCUUUCCUAAAA | UUUUAGGAAAGAGCCCGAUGA | | [5909-5929] 3′UTR |
| 157 | GCAAGAGCCAGGCUGAAACAU | AUGUUUCAGCCUGGCUCUUGC | | [5849-5889] 3′UTR |
| 158 | GCCAGAUAUUCAGUACUCGAU | AUCGAGUACUGAAUAUCUGGC | | [5689-5709] 3′UTR |
| 159 | CCAGCCCUGAUAUUCUUGAUG | CAUCAAGAAUAUCAGGGCUGG | | [5569-5589] 3′UTR |
| 160 | GGUGAAAAGGUACAAUAGUGC | GCACUAUUGUACCUUUUCACC | | [5253-5273] 3′UTR |
| 161 | CAAAGAAGAUAAAGACCACUC | GAGUGGUCUUUAUCUUCUUUG | | [5155-5175] 3′UTR |
| 162 | UGCAUGAUGAUUAUAAGUCCA | UGGACUUAUAAUCAUCAUGCA | | [5056-5076] 3′UTR |
| 163 | GAGUGCAUGAUGAUUAUAAGU | ACUUAUAAUCAUCAUGCACUG | | [5053-5073] 3′UTR |
| 164 | GAAUGUAGAGAAGGCCUGUCA | UGACAGGCCUUCUCUACAUUC | | [4775-4795] ORF |
| 165 | GCACAUUUAUGUCACCCAGCU | AGCUGGGUGACAUAAAUGUGC | | [4544-4564] ORF |
| 166 | GUGGCAUAAAUUUGAGGUGUC | GACACCUCAAAUUUAUGCCAC | | [4322-4342] ORF |
| 167 | CCAAGGCUUUGAGUACAAGUC | GACUUGUACUCAAAGCCUUGG | | [4079-4099] ORF |
| 168 | CGGCAAUCAUCUAUGGAGGUG | CACCUCCAUAGAUGAUUGCCG | | [3961-3981] ORF |
| 169 | GGCCAUCAUGUAUGUCUUCGC | GCGAAGACAUACAUGAUGGCC | | [3800-3820] ORF |
| 170 | CUGGCCAUCAUGUAUGUCUUC | GAAGACAUACAUGAUGGCCAG | | [3798-3818] ORF |
| 171 | CCAGGUAUGACAGGUGUGCUU | AAGCACACCUGUCAUACCUGG | | [3765-3785] ORF |
| 172 | GACUUUCCUCAACCGCUAUGU | ACAUAGCGGUUGAGGAAAGUC | | [3536-3556] ORF |
| 173 | UGCCGCAACCUCAUAACCUUC | GAAGGUUAUGAGGUUGCGGCA | | [3507-3527] ORF |
| 174 | GAGCUGACAUGGGAGGAUUUU | AAAAUCCUCCCAUGUCAGCUC | | [2955-2975] ORF |
| 175 | AGGAGCUGACAUGGGAGGAUU | AAUCCUCCCAUGUCAGCUCCU | | [2953-2973] ORF |
| 176 | UCCCGUCUAAUGUUUACCAUG | CAUGGUAAACAUUAGACGGGA | | [2781-2801] ORF |
| 177 | UCCCAUCAUCAUCCAGCUGCU | AGCAGCUGGAUGAUGAUGGGA | | [2183-2203] ORF |
| 178 | CCUGACUGUGCUUGACUUCUU | AAGAAGUCAAGCACAGUCAGG | | [1955-1975] ORF |
| 179 | CACAGAAGCAAAGAUUUUAAG | CUUAAAAUCUUUGCUUCUGUG | | [127-147] 5′UTR |
| 180 | UGAGGGUGAAGGCAAGAAGUA | UACUUCUUGCCUUCACCCUCA | | [103-123] 5′UTR |
| 181 | CUCCAUACUCAGCUCUUUAAC | GUUAAAGAGCUGAGUAUGGAG | | [5873-5893] 3′UTR |
| 182 | AGGGCACCAGGAAACAAAGAU | AUCUUUGUUUCCUGGUGCCCU | | [5542-5562] 3′UTR |
| 183 | GGUAUUUGCUUGGACUGACAU | AUGUCAGUCCAAGCAAAUACC | | [5333-5353] 3′UTR |
| 184 | UGAAAAGGUACAAUAGUGCUU | AAGCACUAUUGUACCUUUUCA | | [5255-5275] 3′UTR |
| 185 | GUGAAAAGGUACAAUAGUGCU | AGCACUAUUGUACCUUUUCAC | | [5254-5274] 3′UTR |
| 186 | UGUGGAGGUGAAAAGGUACAA | UUGUACCUUUUCACCUCCACA | | [5247-5267] 3′UTR |
| 187 | UGCAGUGCAUGAUGAUUAUAA | UUAUAAUCAUCAUGCACUGCA | | [5051-5071] 3′UTR |
| 188 | AGGAGACUAUGUUCUACAAUU | AAUUGUAGAACAUAGUCUCCU | | [5031-5051] 3′UTR |
| 189 | CAAAUGAGAAAAGGCAGGAGA | UCUCCUGCCUUUUCUCAUUUG | | [5016-5038] 3′UTR |
| 190 | ACAAAUGAGAAAAGGCAGGAG | CUCCUGCCUUUUCUCAUUUGU | | [5015-5035] 3′UTR |
| 191 | GCCUUCUCUUCUGUGCACCUA | UAGGUGCACAGAAGAGAAGGC | | [4885-4905] 3′UTR |
| 192 | CACAUUUAUGUCACCCAGCUG | CAGCUGGGUGACAUAAAUGUG | | [4545-4565] ORF |
| 193 | CCCUUUGCCUCCAUCCUCAAA | UUUGAGGAUGGAGGCAAAGGG | Rat, Mouse | [4374-4394] ORF |
| 194 | GGAGUGGCAUAAAUUUGAGGU | ACCUCAAAUUUAUGCCACUCC | | [4319-4339] ORF |
| 195 | AGCUGCCCACUUUCCACAUCU | AGAUGUGGAAAGUGGGCAGCU | | [3928-3948] ORF |
| 196 | GUGCCGCAACCUCAUAACCUU | AAGGUUAUGAGGUUGCGGCAC | | [3506-3526] ORF |
| 197 | CAGAUCGUGCUUACUACUAUG | CAUAGUAGUAAGCACGAUCUG | | [3373-3393] ORF |
| 198 | GCUGCUGUUUAGUUCUGAAGA | UCUUCAGAACUAAACAGCAGC | | [2354-2374] ORF |
| 199 | GACCUGGUGCUGCUGUUUAGU | ACUAAACAGCAGCACCAGGUC | | [2346-2366] ORF |
| 200 | CAGUCCCAUCAUCAUCCAGCU | AGCUGGAUGAUGAUGGGACUG | | [2180-2200] ORF |
| 201 | AAAGAGAGCGUGAAGAAGGAA | UUCCUUCUUCACGCUCUCUUU | | [2106-2126] ORF |
| 202 | GCAAAGAUUUUAAGAGGAAAG | CUUUCCUCUUAAAAUCUUUGC | | [134-154] 5′UTR |
| 203 | UGAGAAAUGCCAGCUGUCAUU | AAUGACAGCUGGCAUUUCUCA | | [1240-1260] ORF |
| 204 | CCUCCAUACUCAGCUCUUUAA | UUAAAGAGCUGAGUAUGGAGG | | [5872-5892] 3′UTR |
| 205 | CCCUGAUAUUCUUGAUGUCAC | GUGACAUCAAGAAUAUCAGGG | | [5573-5593] 3′UTR |
| 206 | AGCCCUGAUAUUCUUGAUGUC | GACAUCAAGAAUAUCAGGGCU | | [5571-5591] 3′UTR |
| 207 | AUCGAUGCGUUGCUUUGGUAU | AUACCAAAGCAACGCAUCGAU | | [5317-5337] 3′UTR |
| 208 | AGACUAUGUUCUACAAUUGCA | UGCAAUUGUAGAACAUAGUCU | | [5034-5054] 3′UTR |
| 209 | CUCCAUCAGAAUCCACCUUAG | CUAAGGUGGAUUCUGAUGGAG | | [4931-4951] 3′UTR |
| 210 | CCACUAUGAGAACUUCUGAGC | GCUCAGAAGUUCUCAUAGUGG | | [4832-4852] ORF + 3′UTR |
| 211 | CAUGCACCACUAUGAGAACUU | AAGUUCUCAUAGUGGUGCAUG | | [4826-4846] ORF |
| 212 | GGCUACCAUGUUCUUUCCGAC | GUCGGAAAGAACAUGGUAGCC | | [528-548] ORF |
| 213 | GCCAUCAUGUAUGUCUUCGCC | GGCGAAGACAUACAUGAUGGC | | [3801-3821] ORF |
| 214 | CGUCUUUGUGAAUGAUGGGUC | GACCCAUCAUUCACAAAGACG | | [3701-3721] ORF |
| 215 | ACAUCUUCUCAGUCAGCCCAC | GUGGGCUGACUGAGAAGAUGU | | [3652-3672] ORF |
| 216 | GCAGUCAAUGUCUACAUCUUC | GAAGAUGUAGACAUUGACUGC | | [3639-3659] ORF |
| 217 | GCAACCUCAUAACCUUCCUGC | GCAGGAAGGUUAUGAGGUUGC | | [3511-3531] ORF |
| 218 | UCAUGUUCUCUUAUAUCUUGC | GCAAGAUAUAAGAGAACAUGA | | [3478-3498] ORF |
| 219 | UCUCCUUCAUGUUCUCUUAUA | UAUAAGAGAACAUGAAGGAGA | | [3472-3492] ORF |
| 220 | UGAAGAAGAGGUUUGGCAAAA | UUUUGCCAAACCUCUUCUUCA | | [3187-3207] ORF |
| 221 | GUAUGACCUGGUGCUGCUGUU | AACAGCAGCACCAGGUCAUAC | | [2342-2362] ORF |
| 222 | ACUGUGCUUGACUUCUUUGAA | UUCAAAGAAGUCAAGCACAGU | | [1959-1979] ORF |
| 223 | UGACUGUGCUUGACUUCUUUG | CAAAGAAGUCAAGCACAGUCA | | [1957-1977] ORF |
| 224 | GCAAAUUCUCCCGUACAGACU | AGUCUGUACGGGAGAAUUUGC | | [1456-1476] ORF |
| 225 | GAUUUCGGAGUUGGAGGACAA | UUGUCCUCCAACUCCGAAAUC | | [1394-1414] ORF |
| 226 | CCAGAUUUCGGAGUUGGAGGA | UCCUCCAACUCCGAAAUCUGG | | [1391-1411] ORF |
| 227 | AGGCAAGAAGUAGAGCACAGA | UCUGUGCUCUACUUCUUGCCU | | [112-132] 5′UTR |
| 228 | CAAAUCCAGUUUUGGUCCUAC | GUAGGACCAAAACUGGAUUUG | | [6109-6129] 3′UTR |
| 229 | UCCUAAAAAGCUGAGCUGUAA | UUACAGCUCAGCUUUUUAGGA | | [5922-5942] 3′UTR |
| 230 | CUAUUCCCUCCAGCUCCAAAA | UUUUGGAGCUGGAGGGAAUAG | | [5467-5487] 3′UTR |
| 231 | CUGUGAUGCUUAGAACCUGGA | UCCAGGUUCUAAGCAUCACAG | | [5415-5435] 3′UTR |
| 232 | UGGUAUUUGCUUGGACUGACA | UGUCAGUCCAAGCAAAUACCA | | [5332-5352] 3′UTR |
| 233 | UCGAUGCGUUGCUUUGGUAUU | AAUACCAAAGCAACGCAUCGA | | [5318-5338] 3′UTR |
| 234 | GAAAAGGUACAAUAGUGCUUG | CAAGCACUAUUGUACCUUUUC | | [5256-5276] 3′UTR |
| 235 | AGAGUGAGGGUGAAGGCAAGA | UCUUGCCUUCACCCUCACUCU | | [99-119] 5′UTR |
| 236 | UGAGAAAAGGCAGGAGACUAU | AUAGUCUCCUGCCUUUUCUCA | | [5020-5040] 3′UTR |
| 237 | GAAUAUUGACCCAGACAAUUA | UAAUUGUCUGGGUCAAUAUUC | | [4993-5013] 3′UTR |
| 238 | UGCCUUCUCUUCUGUGCACCU | AGGUGCACAGAAGAGAAGGCA | | [4884-4904] 3′UTR |
| 239 | GUAUCCUGCCUUCUCUUCUGU | ACAGAAGAGAAGGCAGGAUAC | | [4878-4898] 3′UTR |
| 240 | CACCACUAUGAGAACUUCUGA | UCAGAAGUUCUCAUAGUGGUG | | [4830-4850] ORF |
| 241 | GCACCACUAUGAGAACUUCUG | CAGAAGUUCUCAUAGUGGUGC | | [4829-4849] ORF |
| 242 | CAUCGCCACCUACCAGAACAU | AUGUUCUGGUAGGUGGCGAUG | | [1070-1090] ORF |
| 243 | CAGAAGUUCUAUUGGUGGUUC | GAACCACCAAUAGAACUUCUG | | [3732-3752] ORF |
| 244 | CUCAACCGCUAUGUGCCUUUU | AAAAGGCACAUAGCGGUUGAG | | [3543-3563] ORF |
| 245 | ACAUCGUGUGUGUGGCAAUCU | AGAUUGCCACACACACGAUGU | | [3325-3345] ORF |
| 246 | GCCGAGAACACAAGAAGCUAC | GUAGCUUCUUGUGUUCUCGGC | | [2074-2094] ORF |
| 247 | CAAUGUCUUUGUCUGGCAUAA | UUAUGCCAGACAAAGACAUUG | | [1874-1894] ORF |
| 248 | CCAUUGUCCUCGACCAGUUUG | CAAACUGGUCGAGGACAAUGG | | [1708-1728] ORF |
| 249 | CAAAUUCUCCCGUACAGACUA | UAGUCUGUACGGGAGAAUUUG | | [1457-1477] ORF |
| 250 | GCAAGAAGUAGAGCACAGAAG | CUUCUGUGCUCUACUUCUUGC | | [114-134] 5′UTR |
| 251 | GGUCAAGACAUAGAAUGUUUC | GAAACAUUCUAUGUCUUGACC | | [5998-6018] 3′UTR |
| 252 | CUAAAAAGCUGAGCUGUAAAA | UUUUACAGCUCAGCUUUUUAG | | [5924-5944] 3′UTR |
| 253 | CAGCCCUGAUAUUCUUGAUGU | ACAUCAAGAAUAUCAGGGCUG | | [5570-5590] 3′UTR |
| 254 | GGAAUUUGUGGUGGCCUCUGA | UCAGAGGCCACCACAAAUUCC | | [1181-1201] ORF |
| 255 | UCAGCAACUUUGAGGAGGGCA | UGCCCUCCUCAAAGUUGCUGA | | [5527-5547] 3′UTR |
| 256 | UGAUGCUUAGAACCUGGACAG | CUGUCCAGGUUCUAAGCAUCA | | [5418-5438] 3′UTR |
| 257 | CUGACAUUUGAGGGAGGAGGA | UCCUCCUCCCUCAAAUGUCAG | | [5347-5367] 3′UTR |
| 258 | GCUUUGGUAUUUGCUUGGACU | AGUCCAAGCAAAUACCAAAGC | | [5328-5348] 3′UTR |
| 259 | GAAAAGGCAGGAGACUAUGUU | AACAUAGUCUCCUGCCUUUUC | | [5023-5043] 3′UTR |
| 260 | UGCACCACUAUGAGAACUUCU | AGAAGUUCUCAUAGUGGUGCA | | [4828-4848] ORF |
| 261 | CAGAAAGUGCUGAACCGGAGU | ACUCCGGUUCAGCACUUUCUG | | [4620-4640] ORF |
| 262 | UGAGUGGCUGGCUGACAUCAU | AUGAUGUCAGCCAGCCACUCA | Rat, Mouse | [4481-4501] ORF |
| 263 | CAUGUAUGUCUUCGCCUCCCA | UGGGAGGCGAAGACAUACAUG | | [3806-3826] ORF |
| 264 | GAGAGAAAGGUGAGAGUGCAG | CUGCACUCUCACCUUUCUCUC | | [48-68] 5′UTR |
| 265 | CCAGCGUCUCCUUCAUGUUCU | AGAACAUGAAGGAGACGCUGG | | [3466-3485] ORF |
| 266 | AGAGGCGCUGCAAGAGAAGAU | AUCUUCUCUUGCAGCGCCUCU | | [3239-3259] ORF |
| 267 | CUGAAGAAGAGGUUUGGCAAA | UUUGCCAAACCUCUUCUUCAG | | [3186-3206] ORF |
| 268 | ACUGAAGAAGAGGUUUGGCAA | UUGCCAAACCUCUUCUUCAGU | | [3185-3205] ORF |
| 269 | GAGGUGGAGGUGGAAAUGGUA | UACCAUUUCCACCUCCACCUC | | [3037-3057] ORF |
| 270 | AGAGGAAAGAAGACAUCUGAA | UUCAGAUGUCUUCUUUCCUCU | | [146-166] 5′UTR |
| 271 | AUCAUUGCUCUCUGCUGCCUU | AAGGCAGCAGAGAGCAAUGAU | | [2007-2027] ORF |
| 272 | UGCAGCCCAAUGUCUUUGUCU | AGACAAAGACAUUGGGCUGCA | | [1887-1887] ORF |
| 273 | CCUGCAGCCCAAUGUCUUUGU | ACAAAGACAUUGGGCUGCAGG | | [1865-1885] ORF |
| 274 | CAGGUCAAGACAUAGAAUGUU | AACAUUCUAUGUCUUGACCUG | | [5996-6016] 3′UTR |
| 275 | GACUGACAUUUGAGGGAGGAG | CUCCUCCCUCAAAUGUCAGUC | | [5345-5365] 3′UTR |
| 276 | GUAUUUGCUUGGACUGACAUU | AAUGUCAGUCCAAGCAAAUAC | | [5334-5354] 3′UTR |
| 277 | GGAUCUCCCAAAGAAGAUAAA | UUUAUCUUCUUUGGGAGAUCC | | [5147-5167] 3′UTR |
| 278 | CCCACUUCAUGCACCACUAUG | CAUAGUGGUGCAUGAAGUGGG | | [4819-4839] ORF |
| 279 | ACCUUGAUGGACCGUUUGGAG | CUCCAAACGGUCCAUCAAGGU | | [4288-4308] ORF |
| 280 | CAGGUAUGACAGGUGUGCUUC | GAAGCACACCUGUCAUACCUG | | [3766-3786] ORF |
| 281 | GCAAUCUUCUCGGCCAUCUGU | ACAGAUGGCCGAGAAGAUUGC | | [3339-3359] ORF |
| 282 | GGUGGUCUUCAUGAAAGGCUC | GAGCCUUUCAUGAAGACCACC | Mouse | [2747-2767] ORF |
| 283 | CAAGAUCCCUAAGGAGUAUGA | UCAUACUCCUUAGGGAUCUUG | | [2327-2347] ORF |
| 284 | AAUGUCUUUGUCUGGCAUAAA | UUUAUGCCAGACAAAGACAUU | | [1875-1895] ORF |
| 285 | CUGCAGCCCAAUGUCUUUGUC | GACAAAGACAUUGGGCUGCAG | | [1866-1886] ORF |
| 286 | GAGGACAACAUAGUGGUUGAA | UUCAACCACUAUGUUGUCCUC | | [1407-1427] ORF |
| 287 | CCAGUUUUGGUCCUACUAAAU | AUUUAGUAGGACCAAAACUGG | | [6114-6134] 3′UTR |
| 288 | CCUAGAAGCAACCACUGAUGU | ACAUCAGUGGUUGCUUCUAGG | | [6079-6099] 3′UTR |
| 289 | UCCUAGAAGCAACCACUGAUG | CAUCAGUGGUUGCUUCUAGGA | | [6078-8096] 3′UTR |
| 290 | GAAUUUGUGGUGGCCUCUGAG | CUCAGAGGCCACCACAAAUUC | | [1182-1202] ORF |
| 291 | AAGGCAGGAGACUAUGUUCUA | UAGAACAUAGUCUCCUGCCUU | | [5026-5046] 3′UTR |
| 292 | GAGAAAAGGCAGGAGACUAUG | CAUAGUCUCCUGCCUUUUCUC | | [5021-5041] 3′UTR |
| 293 | GCUUCCAGUAUCCUGCCUUCU | AGAAGGCAGGAUACUGGAAGC | | [4871-4891] 3′UTR |
| 294 | CUAUGAGAACUUCUGAGCCUG | CAGGCUCAGAAGUUCUCAUAG | | [4835-4855] ORF + 3′UTR |
| 295 | GAAAGUGCUGAACCGGAGUCU | AGACUCCGGUUCAGCACUUUC | | [4622-4642] ORF |
| 296 | GUGUCUGUGCACAUUUAUGUC | GACAUAAAUGUGCACAGACAC | | [4536-4556] ORF |
| 297 | GGACUGAAGAAGAGGUUUGGC | GCCAAACCUCUUCUUCAGUCC | | [3183-3203] ORF |
| 298 | GACAACAUAGUGGUUGAAGAU | AUCUUCAACCACUAUGUUGUC | | [1410-1430] ORF |
| 299 | CCAAAAGGAGCAGCCCUACUU | AAGUAGGGCUGCUCCUUUUGG | | [5462-5502] 3′UTR |
| 300 | CCACGCAGUCAAUGUCUACAU | AUGUAGACAUUGACUGCGUGG | | [3635-3655] ORF |
| 301 | GGUGGAGGUGGAAAUGGUAUU | AAUACCAUUUCCACCUCCACC | | [3039-3059] ORF |
| 302 | GUAGAGCACAGAAGCAAAGAU | AUCUUUGCUUCUGUGCUCUAC | | [121-141] 5′UTR |
| 303 | GGAGAAUAUUGACCCAGACAA | UUGUCUGGGUCAAUAUUCUCC | | [4990-5010] 3′UTR |
| 304 | UGCUUCCAGUAUCCUGCCUUC | GAAGGCAGGAUACUGGAAGCA | | [4870-4890] 3′UTR |
| 305 | UGAGUACAAGUCAGGACAGUG | CACUGUCCUGACUUGUACUCA | | [4088-4108] ORF |
| 306 | GGACGAAUUCUUCACCAUGAU | AUCAUGGUGAAGAAUUCGUCC | | [2834-2854] ORF |
| 307 | CUGCCUUCCCUUAGUGAGUCU | AGACUCACUAAGGGAAGGCAG | | [2021-2041] ORF |
| 308 | CCUCAACCUAAGCAGCUCACA | UGUGAGCUGCUUAGGUUGAGG | | [1906-1928] ORF |
| 309 | GGACAACAUAGUGGUUGAAGA | UCUUCAACCACUAUGUUGUCC | | [1409-1429] ORF |
| 310 | GGAGGACAACAUAGUGGUUGA | UCAACCACUAUGUUGUCCUCC | | [1406-1426] ORF |
| 311 | GGGUGAAGGCAAGAAGUAGAG | CUCUACUUCUUGCCUUCACCC | | [106-126] 5′UTR |
| 312 | CCAGUCAGGAUCUCCCAAAGA | UCUUUGGGAGAUCCUGACUGG | | [5140-5160] 3′UTR |
| 313 | GGGAGAAUAUUGACCCAGACA | UGUCUGGGUCAAUAUUCUCCC | | [4989-5009] 3′UTR |
| 314 | CAGCUGUCGAGUCUCGUUCAU | AUGAACGAGACUCGACAGCUG | | [3083-3103] ORF |
| 315 | UGGGAGCUCUUCUGACUGGAU | AUCCAGUCAGAAGAGCUCCCA | | [238-258] ORF |
| 316 | CAAGAAGGAGAUUGAAGACAU | AUGUCUUCAAUCUCCUUCUUG | | [1787-1807] ORF |
| 317 | CCAAAUCCAGUUUUGGUCCUA | UAGGACCAAAACUGGAUUUGG | | [6108-6128] 3′UTR |
| 318 | UGAGCAGUUCUUCUCUACCAU | AUGGUAGAGAAGAACUGCUCA | | [1199-1219] ORF |
| 319 | UUCCUUUUUCAUCGGGCUCUU | AAGAGCCCGAUGAAAAAGGAA | | [5901-5921] 3′UTR |
| 320 | AGGAUCUCCCAAAGAAGAUAA | UUAUCUUCUUUGGGAGAUCCU | | [5146-5166] 3′UTR |
| 321 | CGCAGUCAAUGUCUACAUCUU | AAGAUGUAGACAUUGACUGCG | | [3638-3658] ORF |
| 322 | CCCAAAAGCUGCAGCAGUACA | UGUACUGCUGCAGCUUUUGGG | | [3277-3297] ORF |
| 323 | AGCUGUCGAGUCUCGUUCAUC | GAUGAACGAGACUCGACAGCU | | [3084-3104] ORF |
| 324 | CGAGGUGGUGGAGUCUAUGUU | AACAUAGACUCCACCACCUCG | | [2906-2926] ORF |
| 325 | GUGCUGCUGUUUAGUUCUGAA | UUCAGAACUAAACAGCAGCAC | | [2352-2372] ORF |
| 326 | UGACCGCUACUGGUUUGAGAA | UUCUCAAACCAGUAGCGGUCA | | [1745-1765] ORF |
| 327 | GUCCUGAACAAGGGUUUUCAA | UUGAAAACCCUUGUUCAGGAC | | [1269-1289] ORF |
| 328 | CAUCGGGCUCUUUCCUAAAAA | UUUUUAGGAAAGAGCCCGAUG | | [5910-5930] 3′UTR |
| 329 | UCCUUUUUCAUCGGGCUCUUU | AAAGAGCCCGAUGAAAAAGGA | | [5902-5922] 3′UTR |
| 330 | GCAGAAGCUGUGAUGCUUAGA | UCUAAGCAUCACAGCUUCUGC | | [5408-5428] 3′UTR |
| 331 | CUAAUUGACUUCCACAAAAUC | GAUUUUGUGGAAGUCAAUUAG | | [5299-5319] 3′UTR |
| 332 | AGAAUAUUGACCCAGACAAUU | AAUUGUCUGGGUCAAUAUUCU | | [4992-5012] 3′UTR |
| 333 | GGGCAGCCAAAUGCUGUGUAA | UUACACAGCAUUUGGCUGCCC | | [4418-4438] ORF |
| 334 | CAAGGACGAAUUCUUCACCAU | AUGGUGAAGAAUUCGUCCUUG | | [2831-2851] ORF |
| 335 | AGGACAACAUAGUGGUUGAAG | CUUCAACCACUAUGUUGUCCU | | [1408-1428] ORF |
| 336 | CUCAGCUCUUUAACUUUUCUU | AAGAAAAGUUAAAGAGCUGAG | | [5880-5900] 3′UTR |
| 337 | GUCUAUUCCCUCCAGCUCCAA | UUGGAGCUGGAGGGAAUAGAC | | [5465-5485] 3′UTR |
| 338 | GUAUACAGGAUACCGUCCUUU | AAAGGACGGUAUCCUGUAUAC | | [1139-1159] ORF |
| 339 | CCAGGAAUGACCAAGAAUGUA | UACAUUCUUGGUCAUUCCUGG | | [4761-4781] ORF |
| 340 | CCUCAGGACCACCAUGCUAUA | UAUAGCAUGGUGGUCCUGAGG | | [4580-4600] ORF |
| 341 | GCGUGUUUGCAGAUCGUGCUU | AAGCACGAUCUGCAAACACGC | | [3364-3384] ORF |
| 342 | GACCAAGGACCCAGAGAGAAA | UUUCUCUCUGGGUCCUUGGUC | | [35-55] 5′UTR |
| 343 | GCGCUAUGACGGCUGGUUUAA | UUAAACCAGCCGUCAUAGCGC | | [311-331] ORF |
| 344 | CUGACAAGGAUGGCAAUGGCU | AGCCAUUGCCAUCCUUGUCAG | Mouse | [2695-2715] ORF |
| 345 | AGACAUCUGAACCCAACACCA | UGGUGUUGGGUUCAGAUGUCU | | [156-176] 5′UTR |
| 346 | CUUCCCUUAGUGAGUCUGCUU | AAGCAGACUCACUAAGGGAAG | | [2025-2045] ORF |
| 347 | GAAUGGGCUGUUCUCCAAGAA | UUCUUGGAGAACAGCCCAUUC | | [1772-1792] ORF |
| 348 | CCGCUACUGGUUUGAGAACAC | GUGUUCUCAAACCAGUAGCGG | | [1748-1768] ORF |
| 349 | CUGGAGUGAUCUCAACCCUAA | UUAGGGUUGAGAUCACUCCAG | | [1568-1588] ORF |
| 350 | GGAGUUGGAGGACAACAUAGU | ACUAUGUUGUCCUCCAACUCC | | [1400-1420] ORF |
| 351 | UCGGAGUUGGAGGACAACAUA | UAUGUUGUCCUCCAACUCCGA | | [1396-1418] ORF |
| 352 | UGAAGGCAAGAAGUAGAGCAC | GUGCUCUACUUCUUGCCUUCA | | [109-129] 5′UTR |
| 353 | CUGAUGUGAUUUCUACCAAAU | AUUUGGUAGAAAUCACAUCAG | | [6093-6113] 3′UTR |
| 354 | UUCAUCGGGCUCUUUCCUAAA | UUUAGGAAAGAGCCCGAUGAA | | [5906-5928] 3′UTR |
| 355 | UGUUCUACAAUUGCAGUGCAU | AUGCACUGCAAUUGUAGAACA | | [5040-5050] 3′UTR |
| 356 | CAGAAAACACUCCCGGAGUAU | AUACUCCGGGAGUGUUUUCUG | | [1122-1142] ORF |
| 357 | GCAGAAAACACUCCCGGAGUA | UACUCCGGGAGUGUUUUCUGC | | [1121-1141] ORF |
| 358 | UCUCCAUCAGAAUCCACCUUA | UAAGGUGGAUUCUGAUGGAGA | | [4930-4950] 3′UTR |
| 359 | UUCUUUGGCUACCAUGUUCUU | AAGAACAUGGUAGCCAAAGAA | | [522-542] ORF |
| 360 | CCUCAACCGCUAUGUGCCUUU | AAAGGCACAUAGCGGUUGAGG | | [3542-3562] ORF |
| 361 | CCUGCGAGAGACUUUCCUCAA | UUGAGGAAAGUCUCUCGCAGG | | [3527-3547] ORF |
| 362 | GCCAUCUGUGUUGGCGUGUUU | AAACACGCCAACACAGAUGGC | | [3351-3371] ORF |
| 363 | CCUGGACUGAAGAAGAGGUUU | AAACCUCUUCUUCAGUCCAGG | | [3180-3200] ORF |
| 364 | UGAGAAUGGCUUCCUCUCCAA | UUGGAGAGGAAGCCAUUCUCA | | [2813-2833] ORF |
| 365 | AGAGGAUAAGUCCCGUCUAAU | AUUAGACGGGACUUAUCCUCU | | [2771-2791] ORF |
| 366 | GGACAUCCUGGUGGUCUUCAU | AUGAAGACCACCAGGAUGUCC | Mouse | [2736-2758] ORF |
| 367 | UGACAAGGAUGGCAAUGGCUA | UAGCCAUUGCCAUCCUUGUCA | Mouse | [2696-2716] ORF |
| 368 | UGCUGCUGUUUAGUUCUGAAG | CUUCAGAACUAAACAGCAGCA | | [2353-2373] ORF |
| 369 | GGAAGCAGCCAAAGAUGGAGU | ACUCCAUCUUUGGCUGCUUCC | | [2123-2143] ORF |
| 370 | CUCUGGAGUGGUGGCCUAUUU | AAAUAGGCCACCACUCCAGAG | | [2048-2068] ORF |
| 371 | GCCAUCACCAUCAUUGCUCUC | GAGAGCAAUGAUGGUGAUGGC | | [1996-2018] ORF |
| 372 | CUGGUCGCUGUUAUCAACAUU | AAUGUUGAUAACAGCGACCAG | | [1833-1853] ORF |
| 373 | UGGAGUGAUCUCAACCCUAAU | AUUAGGGUUGAGAUCACUCCA | | [1569-1589] ORF |
| 374 | ACUGGAGUGAUCUCAACCCUA | UAGGGUUGAGAUCACUCCAGU | | [1567-1587] ORF |
| 375 | GAAGGUCCUGAACAAGGGUUU | AAACCCUUGUUCAGGACCUUC | | [1265-1285] ORF |
| 376 | GCAGUUCUUCUCUACCAUGGU | ACCAUGGUAGAGAAGAACUGC | | [1202-1222] ORF |
| 377 | CUCUGCCAGAUAUUCAGUACU | AGUACUGAAUAUCUGGCAGAG | | [5685-5705] 3′UTR |
| 378 | CACAAAAUCGAUGCGUUGCUU | AAGCAACGCAUCGAUUUUGUG | | [5311-5331] 3′UTR |
| 379 | CAAUAGUGCUUGUCUCCGAUA | UAUCGGAGACAAGCACUAUUG | | [5265-5265] 3′UTR |
| 380 | ACAAUAGUGCUUGUCUCCGAU | AUCGGAGACAAGCACUAUUGU | | [5264-5284] 3′UTR |
| 381 | CGGAGUAUACAGGAUACCGUC | GACGGUAUCCUGUAUACUCCG | | [1135-1155] ORF |
| 382 | ACUCCCGGAGUAUACAGGAUA | UAUCCUGUAUACUCCGGGAGU | | [1130-1150] ORF |
| 383 | UGGGAGAAUAUUGACCCAGAC | GUCUGGGUCAAUAUUCUCCCA | | [4988-5008] 3′UTR |
| 384 | UCCAGUAUCCUGCCUUCUCUU | AAGAGAAGGCAGGAUACUGGA | | [4874-4894] 3′UTR |
| 385 | GCUGACAUCAUCCAAGAGGUG | CACCUCUUGGAUGAUGUCAGC | | [4491-4511] ORF |
| 386 | GCAAUCAUCUAUGGAGGUGAC | GUCACCUCCAUAGAUGAUUGC | | [3963-3983] ORF |
| 387 | CCAUGGCAGCUAUGCUCUGAU | AUCAGAGCAUAGCUGCCAUGG | | [3905-3925] ORF |
| 388 | CCAGAAGUUCUAUUGGUGGUU | AACCACCAAUAGAACUUCUGG | | [3731-3751] ORF |
| 389 | ACGCAGUCAAUGUCUACAUCU | AGAUGUAGACAUUGACUGCGU | | [3637-3657] ORF |
| 390 | GCACAUCGUGUGUGUGGCAAU | AUUGCCACACACACGAUGUGC | | [3323-3343] ORF |
| 391 | GUACAAGCGCUUCGUGGAGAA | UUCUCCACGAAGCGCUUGUAC | | [3293-3313] ORF |
| 392 | AGGAAGGCUGUGACAAAGCAG | CUGCUUUGUCACAGCCUUCCU | | [2469-2489] ORF |
| 393 | GGUGCUGGUGUUUAGUUCUGA | UCAGAACUAAACAGCAGCACC | | [2351-2371] ORF |
| 394 | CUGGUGCUGCUGUUUAGUUCU | AGAACUAAACAGCAGCACCAG | | [2349-2369] ORF |
| 395 | AGAGCACAGAAGCAAAGAUUU | AAAUCUUUGCUUCUGUGCUCU | | [123-143] 5′UTR |
| 396 | GAAGUAGAGCACAGAAGCAAA | UUUGCUUCUGUGCUCUACUUC | | [118-138] 5′UTR |
| 397 | GAGUUGGAGGACAACAUAGUG | CACUAUGUUGUCCUCCAACUC | | [1401-1421] ORF |
| 398 | GGUCCUGAACAAGGGUUUUCA | UGAAAACCCUUGUUCAGGACC | | [1268-1288] ORF |
| 399 | GGAAGGUCCUGAACAAGGGUU | AACCCUUGUUCAGGACCUUCC | | [1264-1284] ORF |
| 400 | CAUACUCAGCUCUUUAACUUU | AAAGUUAAAGAGCUGAGUAUG | | [5876-5898] 3′UTR |
| 401 | CCAGCCUCUCUGCCAGAUAUU | AAUAUCUGGCAGAGAGGCUGG | | [5678-5698] 3′UTR |
| 402 | AGAAGCUGUGAUGCUUAGAAC | GUUCUAAGCAUCACAGCUUCU | | [5410-5430] 3′UTR |
| 403 | CUUCCACAAAAUCGAUGCGUU | AACGCAUCGAUUUUGUGGAAG | | [5307-5327] 3′UTR |
| 404 | CAGUCAGGAUCUCCCAAAGAA | UUCUUUGGGAGAUCCUGACUG | | [5141-5161] 3′UTR |
| 405 | ACCAGUCAGGAUCUCCCAAAG | CUUUGGGAGAUCCUGACUGGU | | [5138-5159] 3′UTR |
| 406 | CCACCUGUUUAUCAACGGCAC | GUGCCGUUGAUAAACAGGUGG | | [5074-5094] 3′UTR |
| 407 | CAAUUGCAGUGCAUGAUGAUU | AAUCAUCAUGCACUGCAAUUG | | [5047-5067] 3′UTR |
| 408 | GCAGGAGACUAUGUUCUACAA | UUGUAGAACAUAGUCUCCUGC | | [5029-5049] 3′UTR |
| 409 | UGCAGAAAACACUCCCGGAGU | ACUCCGGGAGUGUUUUCUGCA | | [1120-1140] ORF |
| 410 | CUCCCAAUAUUGGGAGAAUAU | AUAUGCUCCCAAUAUUGGGAG | | [4978-4998] 3′UTR |
| 411 | CUGCUUCCAGUAUCCUGCCUU | AAGGCAGGAUACUGGAAGCAG | | [4869-4889] 3′UTR |
| 412 | CCCUCCAGGAAUGACCAAGAA | UUCUUGGUCAUUCCUGGAGGG | | [4757-4777] ORF |
| 413 | CAGGACCACCAUGCUAUACAU | AUGUAUAGCAUGGUGGUCCUG | | [4583-4603] ORF |
| 414 | CUGGUGUCUGUGCACAUUUAU | AUAAAUGUGCACAGACACCAG | | [4533-4553] ORF |
| 415 | CAGGAGUGGCAUAAAUUUGAG | CUCAAAUGUAUGCCACUCCUG | | [4317-4337] ORF |
| 416 | CACGCAGUCAAUGUCUACAUC | GAUGUAGACAUUGACUGCGUG | | [3636-3656] ORF |
| 417 | GAGGUGGUGGAGUCUAUGUUC | GAACAUAGACUCCACCACCUC | | [2907-2927] ORF |
| 418 | CCAGAGGAUAAGUCCCGUCUA | UAGACGGGACUUAUCCUCUGG | | [2769-2789] ORF |
| 419 | UGAGCGAGAAGGAGCUAUUUA | UAAAUAGCUCCUUCUCGCUCA | | [2449-2469] ORF |
| 420 | GCUGCUCAAGAUCCCUAAGGA | UCCUUAGGGAUCUUGAGCAGC | | [2321-2341] ORF |
| 421 | GAAGCUACAAAAGAAACUCAA | UUGAGUUUCUUUUGUAGCUUC | | [2087-2107] ORF |
| 422 | CAACCUAAGCAGCUCACAACU | AGUUGUGAGCUGCUUAGGUUG | | [1911-1931] ORF |
| 423 | CUCAACCUAAGCAGCUCACAA | UUGUGAGCUGCUUAGGUUGAG | | [1909-1929] ORF |
| 424 | CCAAGAAGGAGAUUGAAGACA | UGUCUUCAAUCUCCUUCUUGG | | [1785-1806] ORF |
| 425 | UGGUUUGAGAACACCAGGAAU | AUUCCUGGUGUUCUCAAACCA | | [1755-1775] ORF |
| 426 | CUGGUUUGAGAACACCAGGAA | UUCCUGGUGUUCUCAAACCAG | | [1754-1774] ORF |
| 427 | CCCUGUACAACCAGGACCUAU | AUAGGUCCUGGUUGUACAGGG | | [1621-1641] ORF |
| 428 | CUACAUGAGAAAUGCCAGCUG | CAGCUGGCAUUUCUCAUGUAG | | [1235-1255] ORF |
| 429 | UCCAGUUUUGGUCCUACUAAA | UUUAGUAGGACCAAAACUGGA | | [6113-6133] 3′UTR |
| 430 | CAUGUACAUGUUUUACCACCA | UGGUGGUAAAACAUGUACAUG | | [5973-5993] 3′UTR |
| 431 | GGGCUCUUUCCUAAAAAGCUG | CAGCUUUUUAGGAAAGAGCCC | | [5914-5934] 3′UTR |
| 432 | CCAUACUCAGCUCUUUAACUU | AAGUUAAAGAGCUGAGUAUGG | | [5875-5895] 3′UTR |
| 433 | CCAAAGUGUCAGCAACUUUGA | UCAAAGUUGCGGACACUUUGG | | [5519-5539] 3′UTR |
| 434 | CAAAAGGAGCAGCCCUACUUC | GAAGUAGGGCUGCUCCUUUUG | | [5483-5503] 3′UTR |
| 435 | CCACAAAAUCGAUGCGUUGCU | AGCAACGCAUCGAUUUUGUGG | | [5310-5330] 3′UTR |
| 436 | CUACAAUUGCAGUGCAUGAUG | CAUCAUGCACUGCAAUUGUAG | | [5044-5064] 3′UTR |
| 437 | CCAAUAUUGGGAGAAUAUUGA | UCAAUAUUCUCCCAAUAUUGG | | [4981-5001] 3′UTR |
| 438 | CCUCCCAAUAUUGGGAGAAUA | UAUUCUCCCAAUAUUGGGAGG | | [4977-4997] 3′UTR |
| 439 | CCCUCCCAAUAUUGGGAGAAU | AUUCUCCCAAUAUUGGGAGGG | | [4976-4996] 3′UTR |
| 440 | GAAGAGUGAGGGUGAAGGCAA | UUGCCUUCACCCUCACUCUUC | | [97-117] 5′UTR |
| 441 | CCUCCAGGAAUGACCAAGAAU | AUUCUUGGUCAUUCCUGGAGG | | [4758-4778] ORF |
| 442 | GCACUUCCAGAAAGUGCUGAA | UUCAGCACUUUCUGGAAGUGC | | [4613-4633] ORF |
| 443 | GGCAGCCAAAUGCUGUGUAAG | CUUACACAGCAUUUGGCUGCC | | [4419-4439] ORF |
| 444 | CCAUCAGGAGUGGCAUAAAUU | AAUUUAUGCCACUCCUGAUGG | | [4313-4333] ORF |
| 445 | CGAGUUCCUCAACAUCCGCAU | AUGCGGAUGUUGAGGAACUCG | | [581-601] ORF |
| 446 | CCGGCAAUCAUCUAUGGAGGU | ACCUCCAUAGAUGAUUGCCGG | | [3960-3980] ORF |
| 447 | AACCGCUAUGUGCCUUUUGAU | AUCAAAAGGCACAUAGCGGUU | | [3546-3566] ORF |
| 448 | GUGUUUGCAGAUCGUGCUUAC | GUAAGCACGAUCUGCAAACAC | | [3366-3386] ORF |
| 449 | CUGGUUUAACAACCUGAGGCA | UGCCUCAGGUUGUUAAACCAG | | [323-343] ORF |
| 450 | ACGGCUGGUUUAACAACCUGA | UCAGGUUGUUAAACCAGCCGU | | [319-339] ORF |
| 451 | GACGGCUGGUUUAACAACCUG | CAGGUUGUUAAACCAGCCGUC | | [318-338] ORF |
| 452 | CCUGGUGGUCUUCAUGAAAGG | CCUUUCAUGAAGACCACCAGG | Mouse | [2744-2764] ORF |
| 453 | GAUGAGCGAGAAGGAGCUAUU | AAUAGCUCCUUCUCGCUCAUC | | [2447-2467] ORF |
| 454 | GCUCAAGAUCCCUAAGGAGUA | UACUCCUUAGGGAUCUUGAGC | | [2324-2344] ORF |
| 455 | CAAAGAUGGAGUGCCAGCGAU | AUCGCUGGCACUCCAUCUUUG | | [2132-2152] ORF |
| 456 | CCCAAUGUCUUUGUCUGGCAU | AUGCCAGACAAAGACAUUGGG | | [1872-1892] ORF |
| 457 | GGUCGCUGUUAUCAACAUUGA | UCAAUGUUGAUAACAGCGACC | | [1835-1855] ORF |
| 458 | CGUGCUGGUCGCUGUUAUCAA | UUGAUAACAGCGACCAGCACG | | [1829-1849] ORF |
| 459 | GAGAUUGAAGACAUCCGAAAU | AUUUCGGAUGUCUUCAAUCUC | | [1794-1814] ORF |
| 460 | GAGCACAGAAGCAAAGAUUUU | AAAAUCUUUGCUUCUGUGCUC | | [124-144] 5′UTR |
| 461 | CUGUUCUCCAAGAAGGAGAUU | AAUCUCCUUCUUGGAGAACAG | | [1779-1799] ORF |
| 462 | UGCCAUUGUCCUCGACCAGUU | AACUGGUCGAGGACAAUGGCA | | [1706-1726] ORF |
| 463 | AAGGAACUGGAGUGAUCUCAA | UUGAGAUCACUCCAGUUCCUU | | [1562-1582] ORF |
| 464 | CGGAGUUGGAGGACAACAUAG | CUAUGUUGUCCUCCAACUCCG | | [1399-1419] ORF |
| 465 | ACUGAUGUGAUUUCUACCAAA | UUUGGUAGAAAUCACAUCAGU | | [6092-6112] 3′UTR |
| 466 | ACUCAGCUCUUUAACUUUUCU | AGAAAAGUUAAAGAGCUGAGU | | [5879-5899] 3′UTR |
| 467 | CAGAAGCUGUGAUGCUUAGAA | UUCUAAGCAUCACAGCUUCUG | | [5409-5429] 3′UTR |
| 468 | GUGCAGAAGCUGUGAUGCUUA | UAAGCAUCACAGCUUCUGCAC | | [5406-5426] 3′UTR |
| 469 | ACAGGAUACCGGCCUUUCCUA | UAGGAAAGGACGGUAUCCUGU | | [1143-1163] ORF |
| 470 | CCGGAGUAUACAGGAUACCGU | ACGGUAUCCUGUAUACUCCGG | | [1134-1154] ORF |
| 471 | AGAAAACACUCCCGGAGUAUA | UAUACUCCGGGAGUGUUUUCU | | [1123-1143] ORF |
| 472 | CAAUAUUGGGAGAAUAUUGAC | GUCAAUAUUCUCCCAAUAUUG | | [4982-5002] 3′UTR |
| 473 | CCCAAUAUUGGGAGAAUAUUG | CAAUAUUCUCCCAAUAUUGGG | | [4980-5000] 3′UTR |
| 474 | CCAGCUUCCUGCAGAAAACAC | GUGUUUUCUGCAGGAAGCUGG | Rat | [1111-1131] ORF |
| 475 | GCCCACUUCAUGCACCACUAU | AUAGUGGUGCAUGAAGUGGGC | | [4818-4838] ORF |
| 476 | ACCUGGUGUCUGUGCACAUUU | AAAUGUGCACAGACACCAGGU | | [4531-4551] ORF |
| 477 | GGCUGACAUCAUCCAAGAGGU | ACCUCUGGGAUGAUGUCAGCC | | [4490-4510] ORF |
| 478 | CUGUACCUUGAUGGACCGUUU | AAACGGUCCAUCAAGGUACAG | | [4284-4304] ORF |
| 479 | CCCAAGGCUUUGAGUACAAGU | ACUUGUACUCAAAGCCUUGGG | | [4078-4098] ORF |
| 480 | CAAUCAUCUAUGGAGGUGACA | UGUCACCUCCAUAGAUGAUUG | | [3964-3984] ORF |
| 481 | GAGACUUUCCUCAACCGCUAU | AUAGCGGUUGAGGAAAGUCUC | | [3534-3554] ORF |
| 482 | CGUCUCCUUCAUGUUCUCUUA | UAAGAGAACAUGAAGGAGACG | | [3470-3490] ORF |
| 483 | GCGUCUCCUUCAUGUUCUCUU | AAGAGAACAUGAAGGAGACGC | | [3469-3489] ORF |
| 484 | GCAGAUCGUGCUUACUACUAU | AUAGUAGUAAGCACGAUCUGC | | [3372-3392] ORF |
| 485 | GGAUUUUCACUUCAUGCUGCG | CGCAGCAUGAAGUGAAAAUCC | | [2969-2989] ORF |
| 486 | CUUCACCAUGAUGCGAUCCUU | AAGGAUCGCAUCAUGGUGAAG | | [2843-2863] ORF |
| 487 | CGAAUUCUUCACCAUGAUGCG | CGCAUCAUGGUGAAGAAUUCG | | [2837-2857] ORF |
| 488 | GGAUAAGUCCCGUCUAAUGUU | AACAUUAGACGGGACUUAUCC | | [2774-2794] ORF |
| 489 | CCCUAAGGAGUAUGACCUGGU | ACCAGGUCAUACUCCUUAGGG | | [2333-2353] ORF |
| 490 | CUCAAGAUCCCUAAGGAGUAU | AUACUCCUUAGGGAUCUUGAG | | [2325-2345] ORF |
| 491 | AGAGGAGCAGUCCCAUCAUCA | UGAUGAUGGGACUGCUCCUCU | | [2173-2193] ORF |
| 492 | CCAUCACCAUCAUUGCUCUCU | AGAGAGCAAUGAUGGUGAUGG | | [1999-2019] ORF |
| 493 | CCUAAGCAGCUCACAACUGAC | GUCAGUUGUGAGCUGCUUAGG | | [1914-1934] ORF |
| 494 | GUCGCUGUUAUCAACAUUGAC | GUCAAUGUUGAUAACAGCGAC | | [1836-1856] ORF |
| 495 | GGAAUGGGCUGUUCUCCAAGA | UCUUGGAGAACAGCCCAUUCC | | [1771-1791] ORF |
| 496 | GGAGUGAUCUCAACCCUAAUG | CAUUAGGGUUGAGAUCACUCC | | [1570-1590] ORF |
| 497 | CCCAAGGAACUGGAGUGAUCU | AGAUCACUCCAGUUCCUUGGG | | [1559-1579] ORF |
| 498 | AAGGGUUUUCAAAGCUCCCAA | UUGGGAGCUUUGAAAACCCUU | | [1278-1298] ORF |
| 499 | GCAACCACUGAUGUGAUUUCU | AGAAAUCACAUCAGUGGUUGC | | [6086-8106] 3′UTR |
| 500 | CACCCAGGUCAAGACAUAGAA | UUCUAUGUCUUGACCUGGGUG | | [5992-6012] 3′UTR |

TABLE K
19-mer oligomers for NOXO1 (NADPH oxidase organizer 1)

| | | | | | | |
|---|---|---|---|---|---|---|
| | | | Other | Human-34222190 | Human-41281827 | Human-41281810 |
| Number | Sense siRNA | AntiSense siRNA | Sp | ORF: 261-1373 | ORF: 5-1135 | ORF: 5-1120 |

| | | | | | |
|---|---|---|---|---|---|
| 1 | CGACGAUCACUGGCUUCUU | AAGAAGCCAGUGAUCGUCG | [595-813] ORF | [357-375] ORF | [342-360] ORF |
| 2 | GCGUGUUGGAAACGUCAGA | UCUGACGUUUCCAACACGC | [1036-1054] ORF | [798-816] ORF | [783-801] ORF |
| 3 | CCAAACGUUUGCCUUCUCU | AGAGAAGGCAAACGUUUGG | [323-341] ORF | [67-85] ORF | [67-85] ORF |
| 4 | CAGAUCAAGAGGCUCCAAA | UUUGGAGCCUCUUGAUCUG | [309-327] ORF | [53-71] ORF | [53-71] ORF |
| 5 | GCAGAUCAAGAGGCUCCAA | UUGGAGCCUCUUGAUCUGC | [308-326] ORF | [52-70] ORF | [52-70] ORF |
| 6 | UGCUGUUCCUCUGAGUAAA | UUUACUCAGAGGAACAGCA | [1475-1493] 3′UTR | — | — |
| 7 | GCUGUUGGAAACCUAUUCU | AGAAUAGGUUUCCAACAGC | [536-654] ORF | [296-316] ORF | [283-301] ORF |
| 8 | GGAGUUGGGACGAAUUCAG | CUGAAUUCGUCCCAACUCC | [379-397] ORF | [123-141] ORF | [123-141] ORF |
| 9 | UCCAAACGUUUGCCUUCUC | GAGAAGGCAAACGUUUGGA | [322-340] ORF | [66-84] ORF | [66-84] ORF |
| 10 | CUCCAAACGUUUGCCUUCU | AGAAGGCAAACGUUUGGAG | [321-339] ORF | [65-83] ORF | [65-83] ORF |
| 11 | AGCUGUUGGAAACCUAUUC | GAAUAGGUUUCCAACAGCU | [535-553] ORF | [297-315] ORF | [282-300] ORF |
| 12 | CGAAUUCAGGCAGCUCAAG | CUUGAGCUGCCUGAAUUCG | [389-407] ORF | [133-151] ORF | [133-151] ORF |
| 13 | ACGAAUUCAGGCAGCUCAA | UUGAGCUGCCUGAAUUCGU | [388-406] ORF | [132-150] ORF | [132-150] ORF |
| 14 | CAAGAAAGCUGCAAAGGAC | GUCCUUUGCAGCUUUCUUG | [87-105] 5′UTR | — | — |
| 15 | CGUGUUGGAAACGUCAGAC | GUCUGACGUUUCCAACACG | [1037-1055] ORF | [799-817] ORF | [784-802] ORF |
| 16 | CUGUUGGAAACCUAUUCUC | GAGAAUAGGUUUCCAACAG | [537-555] ORF | [299-317] ORF | [284-302] ORF |
| 17 | GACGAAUUCAGGCAGCUCA | UGAGCUGCCUGAAUUCGUC | [387-405] ORF | [131-149] ORF | [131-149] ORF |
| 18 | GCUGUUCCUCUGAGUAAAG | CUUUACUCAGAGGAACAGC | [1476-1494] 3′UTR | — | — |
| 19 | UGGUGCAGAUCAAGAGGCU | AGCCUCUUGAUCUGCACCA | [304-322] ORF | [48-66] ORF | [48-66] ORF |
| 20 | UUGAGGUGUUGCCAGGAUU | AAUCCUGGCAACACCUCAA | [140-158] 5′UTR | — | — |
| 21 | GCAGCUGUUGGAAACCUAU | AUAGGUUUCCAACAGCUGC | [533-551] ORF | [295-313] ORF | [280-298] ORF |
| 22 | CCUCGCCCAUUUCAGGAAU | AUUCCUGAAAUGGGCGAGG | [220-238] 5′UTR | — | — |
| 23 | GCCAUUUGCUGUUCCUCUG | CAGAGGAACAGCAAAUGGC | [1459-1487] 3′UTR | — | — |
| 24 | UCAAGACCCUCAAGGAGAC | GUCUCCUUGAGGGUCUUGA | [403-421] ORF | [152-168](17/17) | [152-168](17/17) |
| | | | | ORF | ORF |
| 25 | CCAUUUGCUGUUCCUCUGA | UCAGAGGAACAGCAAAUGG | [1470-1488] 3′UTR | — | — |
| 26 | UCGCCCAUUUCAGGAAUCU | AGAUUCCUGAAAUGGGCGA | [222-240] 5′UTR | — | — |
| 27 | CUCGCCCAUUUCAGGAAUC | GAUUCCUGAAAUGGGCGAG | [221-239] 5′UTR | — | — |
| 28 | AGCUUCUCGAUGCACCACU | AGUGGUGCAUCGAGAAGCU | [472-490] ORF | [242-252](11/11) | [219-237] ORF |
| | | | | ORF | |
| 29 | AUACCCAGUUUCAGUGCAA | UUGCACUGAAACUGGGUAU | [275-293] ORF | [19-37] ORF | [19-37] ORF |
| 30 | CCGAUACCCAGUUUCAGUG | CACUGAAACUGGGUAUCGG | [272-290] ORF | [16-34] ORF | [16-34] ORF |
| 31 | AGGAAUCUGCAGCCUGGAA | UUCCAGGCUGCAGAUUCCU | [233-251] 5′UTR | — | — |
| 32 | CGCCCAUUUCAGGAAUCUG | CAGAUUCCUGAAAUGGGCG | [223-241] 5′UTR | — | — |
| 33 | AGACCCUCAAGGAGACCUU | AAGGUCUCCUUGAGGGUCU | [406-424] ORF | [153-171] ORF | [153-171] ORF |
| 34 | CCAUUUCAGGAAUCUGCAG | CUGCAGAUUCCUGAAAUGG | [226-244] 5′UTR | — | — |
| 35 | CCCAUUUCAGGAAUCUGCA | UGCAGAUUCCUGAAAUGGG | [225-243] 5′UTR | — | — |
| 36 | ACAGCAAGAAAGCUGCAAA | UUUGCAGCUUUCUUGCUGU | [83-101] 5′UTR | — | — |
| 37 | CCCGAUACCCAGUUUCAGU | ACUGAAACUGGGUAUCGGG | [271-289] ORF | [15-33] ORF | [15-33] ORF |
| 38 | UGGAAACCUAUUCUCGGAG | CUCCGAGAAUAGGUUUCCA | [541-559] ORF | [303-321] ORF | [288-306] ORF |
| 39 | UCCUCGCCCAUUUCAGGAA | UUCCUGAAAUGGGCGAGGA | [219-237] 5′UTR | — | — |
| 40 | CUUGAGGUGUUGCCAGGAU | AUCCUGGCAACACCUCAAG | [139-157] 5′UTR | — | — |
| 41 | GCCCAUUUCAGGAAUCUGC | GCAGAUUCCUGAAAUGGGC | [224-242] 5′UTR | — | — |
| 42 | CAGCAAGAAAGCUGCAAAG | CUUUGCAGCUUUCUUGCUG | [84-102] 5′UTR | — | — |
| 43 | GGCUCCAAACGUUUGCCUU | AAGGCAAACGUUUGGAGCC | [319-337] ORF | [63-81] ORF | [63-81] ORF |
| 44 | UGCCAGGAUUCUAUCCCAG | CUGGGAUAGAAUCCUGGCA | [149-167] 5′UTR | — | — |
| 45 | GGAAACCUAUUCUCGGAGG | CCUCCGAGAAUAGGUUUCC | [542-560] ORF | [304-322] ORF | [289-307] ORF |
| 46 | CAGCUGUUGGAAACCUAUU | AAUAGGUUUCCAACAGCUG | [534-552] ORF | [296-314] ORF | [281-299] ORF |
| 47 | GACAGCAAGAAAGCUGCAA | UUGCAGCUUUCUUGCUGUC | [82-100] 5′UTR | — | — |
| 48 | UGCAGCUGUUGGAAACCUA | UAGGUUUCCAACAGCUGCA | [532-550] ORF | [294-312] ORF | [279-297] ORF |
| 49 | AGGCUCCAAACGUUUGCCU | AGGCAAACGUUUGGAGCCU | [318-336] ORF | [62-80] ORF | [62-80] ORF |
| 50 | GGACAGCAAGAAAGCUGCA | UGCAGCUUUCUUGCUGUCC | [81-99] 5′UTR | — | — |
| 51 | CUGCAGCUGUUGGAAACCU | AGGUUUCCAACAGCUGCAG | [531-549] ORF | [293-311] ORF | [278-296] ORF |
| 52 | GGCCAUUUGCUGUUCCUCU | AGAGGAACAGCAAAUGGCC | [1468-1486] 3′UTR | — | — |

TABLE L
21-mer oligomers for NOXO1 (NADPH oxidase organizer 1)

| | | | | | | |
|---|---|---|---|---|---|---|
| | | | Other | Human-34222190 | Human-41281827 | Human-41281810 |
| Number | Sense siRNA | AntiSense siRNA | Sp | ORF: 261-1373 | ORF: 5-1135 | ORF: 5-1120 |

| | | | | | |
|---|---|---|---|---|---|
| 1 | GGACGAAUUCAGGCAGCUCAA | UUGAGCUGCCUGAAUUCGUCC | [386-406] ORF | [130-150] ORF | [130-150] ORF |
| 2 | GCAGAUCAAGAGGCUCCAAAC | GUUUGGAGCCUCUUGAUCUGC | [308-328] ORF | [52-72] ORF | [52-72] ORF |
| 3 | GCUUCUCGAUGCACCACUGUU | AACAGUGGUGCAUCGAGAAGC | [473-493] ORF | [242-255](14/14) | [220-240] ORF |
| | | | | ORF | |
| 4 | CGAAUUCAGGCAGCUCAAGAC | GUCUUGAGCUGCCUGAAUUCG | [389-409] ORF | [133-152](20/20) | [133-152](20/20) |
| | | | | ORF | ORF |
| 5 | GCUCCAAACGUUUGCCUUCUC | GAGAAGGCAAACGUUUGGAGC | [320-340] ORF | [64-84] ORF | [64-84] ORF |
| 6 | CUCCAAACGUUUGCCUUCUCU | AGAGAAGGCAAACGUUUGGAG | [321-341] ORF | [65-85] ORF | [65-85] ORF |
| 7 | AUCAAGAGGCUCCAAACGUUU | AAACGUUUGGAGCCUCUUGAU | [312-332] ORF | [56-76] ORF | [56-76] ORF |
| 8 | GUGCAGAUCAAGAGGCUCCAA | UUGGAGCCUCUUGAUCUGCAC | [306-326] ORF | [50-70] ORF | [50-70] ORF |
| 9 | AGCUGUUGGAAACCUAUUCUC | GAGAAUAGGUUUCCAACAGCU | [535-555] ORF | [297-317] ORF | [282-302] ORF |
| 10 | CAUUUGCUGUUCCUCUGAGUA | UACUCAGAGGAACAGCAAAUG | [1471-1491] | — | — |
| | | | 3′UTR | | |
| 11 | UCCAAACGUUUGCCUUCUCUG | CAGAGAAGGCAAACGUUUGGA | [322-342] ORF | [66-86] ORF | [66-86] ORF |
| 12 | UGCAGAUCAAGAGGCUCCAAA | UUUGGAGCCUCUUGAUCUGCA | [307-327] ORF | [51-71] ORF | [51-71] ORF |
| 13 | GCUUGAGGUGUUGCCAGGAUU | AAUCCUGGCAACACCUCAAGC | [138-158] | — | — |
| | | | 5′UTR | | |
| 14 | GCAGGAGUUGGGACGAAUUCA | UGAAUUCGUCCCAACUCCUGC | [376-396] ORF | [120-140] ORF | [120-140] ORF |
| 15 | UGAGGUGUUGCCAGGAUUCUA | UAGAAUCCUGGCAACACCUCA | [141-161] | — | — |
| | | | 5′UTR | | |
| 16 | CCAUUUGCUGUUCCUCUGAGU | ACUCAGAGGAACAGCAAAUGG | [1470-1490] | — | — |
| | | | 3′UTR | | |
| 17 | CGAUACCCAGUUUCAGUGCAA | UUGCACUGAAACUGGGUAUCG | [273-293] ORF | [17-37] ORF | [17-37] ORF |
| 18 | CCAUUUCAGGAAUCUGCAGCC | GGCUGCAGAUUCCUGAAAUGG | [226-246] | — | — |
| | | | 5′UTR | | |
| 19 | GGACAGCAAGAAAGCUGCAAA | UUUGCAGCUUUCUUGCUGUCC | [81-101] 5′UTR | — | — |
| 20 | CGCCCAUUUCAGGAAUCUGCA | UGCAGAUUCCUGAAAUGGGCG | [223-243] | — | — |
| | | | 5′UTR | | |
| 21 | GCCAUUUGCUGUUCCUCUGAG | CUCAGAGGAACAGCAAAUGGC | [1469-1489] | — | — |
| | | | 3′UTR | | |
| 22 | GCAGCUGUUGGAAACCUAUUC | GAAUAGGUUUCCAACAGCUGC | [533-553] ORF | [295-315] ORF | [280-300] ORF |
| 23 | AGCUUCUCGAUGCACCACUGU | ACAGUGGUGCAUCGAGAAGCU | [472-492] ORF | [242-254](13/13) | [219-239] ORF |
| | | | | ORF | |
| 24 | GCUGUUCCUCUGAGUAAAGCU | AGCUUUACUCAGAGGAACAGC | [1476-1496] | — | — |
| | | | 3′UTR | | |
| 25 | CAAGACCCUCAAGGAGACCUU | AAGGUCUCCUUGAGGGUCUUG | [404-424] ORF | [152-171](20/20) | [152-171](20/20) |
| | | | | ORF | ORF |
| 26 | CCUCGCCCAUUUCAGGAAUCU | AGAUUCCUGAAAUGGGCGAGG | [220-240] | — | — |
| | | | 5′UTR | | |
| 27 | GAGGUGUUGCCAGGAUUCUAU | AUAGAAUCCUGGCAACACCUC | [142-162] | — | — |
| | | | 5′UTR | | |
| 28 | GCAAGAAAGCUGCAAAGGACU | AGUCCUUUGCAGCUUUCUUGC | [86-106] 5′UTR | — | — |
| 29 | GGGACAGCAAGAAAGCUGCAA | UUGCAGCUUUCUUGCUGUCCC | [80-100] 5′UTR | — | — |
| 30 | CCGAUACCCAGUUUCAGUGCA | UGCACUGAAACUGGGUAUCGG | [272-292] ORF | [16-36] ORF | [16-36] ORF |
| 31 | CUCGCCCAUUUCAGGAAUCUG | CAGAUUCCUGAAAUGGGCGAG | [221-241] | — | — |
| | | | 5′UTR | | |
| 32 | GUCCUCGCCCAUUUCAGGAAU | AUUCCUGAAAUGGGCGAGGAC | [218-238] | — | — |
| | | | 5′UTR | | |
| 33 | AGGUGUUGCCAGGAUUCUAUC | GAUAGAAUCCUGGCAACACCU | [143-163] | — | — |
| | | | 5′UTR | | |
| 34 | GGAAACCUAUUCUCGGAGGCU | AGCCUCCGAGAAUAGGUUUCC | [542-562] ORF | [304-324] ORF | [289-309] ORF |
| 35 | GCCCAUUUCAGGAAUCUGCAG | CUGCAGAUUCCUGAAAUGGGC | [224-244] | — | — |
| | | | 5′UTR | | |
| 36 | CAGCUGUUGGAAACCUAUUCU | AGAAUAGGUUUCCAACAGCUG | [534-554] ORF | [296-316] ORF | [281-301] ORF |
| 37 | CCUGCAGCUGUUGGAAACCUA | UAGGUUUCCAACAGCUGCAGG | [530-550] ORF | [292-312] ORF | [277-297] ORF |
| 38 | UCCUCGCCCAUUUCAGGAAUC | GAUUCCUGAAAUGGGCGAGGA | [219-239] | — | — |
| | | | 5′UTR | | |
| 39 | GGCUCCAAACGUUUGCCUUCU | AGAAGGCAAACGUUUGGAGCC | [319-339] ORF | [63-83] ORF | [63-83] ORF |
| 40 | GAGGCUCCAAACGUUUGCCUU | AAGGCAAACGUUUGGAGCCUC | [317-337] ORF | [61-81] ORF | [61-81] ORF |
| 41 | CUGCAGCUGUUGGAAACCUAU | AUAGGUUUCCAACAGCUGCAG | [531-551] ORF | [293-313] ORF | [278-298] ORF |
| 42 | GACAGCAAGAAAGCUGCAAAG | CUUUGCAGCUUUCUUGCUGUC | [82-102] 5′UTR | — | — |
| 43 | CCAAACGUUUGCCUUCUCUGU | ACAGAGAAGGCAAACGUUUGG | [323-343] ORF | [67-87] ORF | [67-87] ORF |
| 44 | UGCAGCUGUUGGAAACCUAUU | AAUAGGUUUCCAACAGCUGCA | [532-552] ORF | [294-314] ORF | [279-299] ORF |
| 45 | GGCCAUUUGCUGUUCCUCUGA | UCAGAGGAACAGCAAAUGGCC | [1468-1488] | — | — |
| | | | 3′UTR | | |
| 46 | UCAGGAAUCUGCAGCCUGGAA | UUCCAGGCUGCAGAUUCCUGA | [231-251] | — | — |
| | | | 5′UTR | | |

TABLE M
19-mer oligomers for NOXO2 (NADPH oxidase organizer 2, NCF1)

| | | | | |
|---|---|---|---|---|
| | | | | Human-115298671 |
| Number | Sense siRNA | AntiSense siRNA | Other Sp | ORF: 71-1243 |

| | | | | |
|---|---|---|---|---|
| 1 | CCUGUACAUACGUGUUCUA | UAGAACACGUAUGUACAGG | | [1292-1310] 3′UTR |
| 2 | GGGCGAUCAAUCCAGAGAA | UUCUCUGGAUUGAUCGCCC | | [258-276] ORF |
| 3 | GUACAUACGUGUUCUAUAG | CUAUAGAACACGUAUGUAC | | [1295-1313] 3′UTR |
| 4 | CCAUUGCCAACUACGAGAA | UUCUCGUAGUUGGCAAUGG | | [558-576] ORF |
| 5 | ACAUACGUGUUCUAUAGAG | CUCUAUAGAACACGUAUGU | | [1297-1315] 3′UTR |
| 6 | CAAAAAAGCCAGAGACAUA | UAUGUCUCUGGCUUUUUUG | | [468-486] ORF |
| 7 | ACAAAAAAGCCAGAGACAU | AUGUCUCUGGCUUUUUUGU | | [467-485] ORF |
| 8 | GCCUGUACAUACGUGUUCU | AGAACACGUAUGUACAGGC | | [1291-1309] 3′UTR |
| 9 | CCGUCCAUGUACCUGCAAA | UUUGCAGGUACAUGGACGG | | [896-914] ORF |
| 10 | CUGUACAUACGUGUUCUAU | AUAGAACACGUAUGUACAG | | [1293-1311] 3′UTR |
| 11 | UCAGAUGAAAGCAAAGCGA | UCGCUUUGCUUUCAUCUGA | | [658-676] ORF |
| 12 | CGAUCAAUCCAGAGAACAG | CUGUUCUCUGGAUUGAUCG | | [261-279] ORF |
| 13 | CGUCCAUGUACCUGCAAAA | UUUUGCAGGUACAUGGACG | | [897-915] ORF |
| 14 | GUGGUUCUGUCAGAUGAAA | UUUCAUCUGACAGAACCAC | | [649-667] ORF |
| 15 | GCCAACUACGAGAAGACCU | AGGUCUUCUCGUAGUUGGC | | [563-581] ORF |
| 16 | ACAUACUUGAUGCCCAAAG | CUUUGGGCAUCAAGUAUGU | | [482-500] ORF |
| 17 | CCAGAGACAUACUUGAUGC | GCAUCAAGUAUGUCUCUGG | | [476-494] ORF |
| 18 | GGACAACCAGACAAAAAAG | CUUUUUUGUCUGGUUGUCC | | [457-475] ORF |
| 19 | GCGAUCAAUCCAGAGAACA | UGUUCUCUGGAUUGAUCGC | | [260-278] ORF |
| 20 | UGCCUGUACAUACGUGUUC | GAACACGUAUGUACAGGCA | | [1290-1308] 3′UTR |
| 21 | GCCAUUGCCAACUACGAGA | UCUCGUAGUUGGCAAUGGC | | [557-575] ORF |
| 22 | AGCCAGAGACAUACUUGAU | AUCAAGUAUGUCUCUGGCU | | [474-492] ORF |
| 23 | AAAAGCCAGAGACAUACUU | AAGUAUGUCUCUGGCUUUU | | [471-489] ORF |
| 24 | GGUGGUUCUGUCAGAUGAA | UUCAUCUGACAGAACCACC | | [648-666] ORF |
| 25 | UGUACAUGUUCCUGGUGAA | UUCACCAGGAACAUGUACA | Guinea-pig | [144-162] ORF |
| 26 | GUGCCUGUACAUACGUGUU | AACACGUAUGUACAGGCAC | | [1289-1307] 3′UTR |
| 27 | GCUACUUCCCGUCCAUGUA | UACAUGGACGGGAAGUAGC | | [888-906] ORF |
| 28 | GUGUACAUGUUCCUGGUGA | UCACCAGGAACAUGUACAC | Guinea-pig | [143-161] ORF |
| 29 | CGUGCCUGUACAUACGUGU | ACACGUAUGUACAGGCACG | | [1288-1306] 3′UTR |
| 30 | UGGUGGGUCAUCAGGAAAG | CUUUCCUGAUGACCCACCA | | [857-875] ORF |
| 31 | ACACCUUCAUCCGUCACAU | AUGUGACGGAUGAAGGUGU | | [78-96] ORF |
| 32 | UGGUGGUUCUGUCAGAUGA | UCAUCUGACAGAACCACCA | | [647-665] ORF |
| 33 | UGGUGGAGGUCGUAGAGAA | UUCUCUACGACCUCCACCA | | [615-633] ORF |
| 34 | GUCGUAGAGAAGAGCGAGA | UCUCGCUCUUCUCUACGAC | | [623-641] ORF |
| 35 | AGAGUACCGCGACAGACAU | AUGUCUGUCGCGGUACUCU | | [507-525] ORF |
| 36 | ACGAGUUCCAUAAAACCUU | AAGGUUUUAUGGAACUCGU | | [213-231] ORF |
| 37 | GGUUGGUGGUUCUGUCAGA | UCUGACAGAACCACCAACC | | [644-662] ORF |
| 38 | GCAACAGCGUCCGUUUUCU | AGAAAACGGACGCUGUUGC | | [1047-1065] ORF |
| 39 | GUUGGUGGUUCUGUCAGAU | AUCUGACAGAACCACCAAC | | [645-663] ORF |
| 40 | UGGAGGUCGUAGAGAAGAG | CUCUUCUCUACGACCUCCA | | [618-636] ORF |
| 41 | CCAGACAAAAAAGCCAGAG | CUCUGGCUUUUUUGUCUGG | | [463-481] ORF |
| 42 | CCACGGACAACCAGACAAA | UUUGUCUGGUUGUCCGUGG | | [453-471] ORF |
| 43 | AUCUACGAGUUCCAUAAAA | UUUUAUGGAACUCGUAGAU | Rat, Mouse | [209-227] ORF |
| 44 | UGUCGGAGAAGGUGGUCUA | UAGACCACCUUCUCCGACA | Rat, Mouse | [174-192] ORF |
| 45 | CUUCCUCUUUCCAGUGCAU | AUGCACUGGAAAGAGGAAG | | [4-22] 5′UTR |
| 46 | AGGUCAUUCACAAGCUCCU | AGGAGCUUGUGAAUGACCU | | [831-849] ORF |
| 47 | CAGACAAAAAAGCCAGAGA | UCUCUGGCUUUUUUGUCUG | | [464-482] ORF |
| 48 | CAGCUCCCAAGUGGUUUGA | UCAAACCACUUGGGAGCUG | | [297-315] ORF |
| 49 | GCCAGCACUAUGUGUACAU | AUGUACACAUAGUGCUGGC | Mouse | [132-150] ORF |
| 50 | CGGACAACCAGACAAAAAA | UUUUUUGUCUGGUUGUCCG | | [456-474] ORF |
| 51 | GAGUUCCAUAAAACCUUAA | UUAAGGUUUUAUGGAACUC | | [215-233] ORF |
| 52 | AGACAAAAAAGCCAGAGAC | GUCUCUGGCUUUUUUGUCU | | [465-483] ORF |
| 53 | ACCAGACAAAAAAGCCAGA | UCUGGCUUUUUUGUCUGGU | | [462-480] ORF |
| 54 | ACGGACAACCAGACAAAAA | UUUUUGUCUGGUUGUCCGU | | [455-473] ORF |
| 55 | UCAGGAAAGACGACGUCAC | GUGACGUCGUCUUUCCUGA | | [867-885] ORF |
| 56 | CCAACUACGAGAAGACCUC | GAGGUCUUCUCGUAGUUGG | | [564-582] ORF |
| 57 | AGACAUACUUGAUGCCCAA | UUGGGCAUCAAGUAUGUCU | | [480-498] ORF |
| 58 | GGCACACUUACCGAGUACU | AGUACUCGGUAAGUGUGCC | | [344-362] ORF |
| 59 | CCUCUUUCCAGUGCAUUUA | UAAAUGCACUGGAAAGAGG | | [7-25] 5′UTR |
| 60 | UUCUGUCAGAUGAAAGCAA | UUGCUUUCAUCUGACAGAA | | [653-671] ORF |
| 61 | CACGGACAACCAGACAAAA | UUUUGUCUGGUUGUCCGUG | | [454-472] ORF |
| 62 | CGACUUCCUCUUUCCAGUG | CACUGGAAAGAGGAAGUCG | | [1-19] 5′UTR |
| 63 | UCAAUCCAGAGAACAGGAU | AUCCUGUUCUCUGGAUUGA | | [264-282] ORF |
| 64 | ACCUCCUCGACUUCUUCAA | UUGAAGAAGUCGAGGAGGU | | [408-426] ORF |
| 65 | CUCUUUCCAGUGCAUUUAA | UUAAAUGCACUGGAAAGAG | | [8-26] 5′UTR |
| 66 | UCUGUCAGAUGAAAGCAAA | UUUGCUUUCAUCUGACAGA | | [654-672] ORF |
| 67 | CCCAAAGAUGGCAAGAGUA | UACUCUUGCCAUCUUUGGG | | [494-512] ORF |
| 68 | UGAAAUGGCAGGACCUGUC | GACAGGUCCUGCCAUUUCA | | [159-177] ORF |
| 69 | AGCACUAUGUGUACAUGUU | AACAUGUACACAUAGUGCU | Mouse | [135-153] ORF |
| 70 | CCAGCACUAUGUGUACAUG | CAUGUACACAUAGUGCUGG | Mouse | [133-151] ORF |
| 71 | UGGGCUUUGAGAAGCGCUU | AAGCGCUUCUCAAAGCCCA | | [105-123] ORF |
| 72 | UGAUGCCCAAAGAUGGCAA | UUGCCAUCUUUGGGCAUCA | | [489-507] ORF |
| 73 | CAACCAGACAAAAAAGCCA | UGGCUUUUUUGUCUGGUUG | | [460-478] ORF |
| 74 | CCAGAGAACAGGAUCAUCC | GGAUGAUCCUGUUCUCUGG | | [269-287] ORF |
| 75 | GCAAAAGUCAGGGCAAGAC | GUCUUGCCCUGACUUUUGC | | [910-928] ORF |
| 76 | GCUUUGAGAAGCGCUUCGU | ACGAAGCGCUUCUCAAAGC | | [106-126] ORF |
| 77 | AGCCCAACUAUGCAGGUGA | UCACCUGCAUAGUUGGGCU | | [738-756] ORF |
| 78 | GAGACAUACUUGAUGCCCA | UGGGCAUCAAGUAUGUCUC | | [479-497] ORF |
| 79 | UCCUCGACUUCUUCAAGGU | ACCUUGAAGAAGUCGAGGA | | [411-429] ORF |
| 80 | UCCUCUUUCCAGUGCAUUU | AAAUGCACUGGAAAGAGGA | | [6-24] 5′UTR |
| 81 | GCACUAUGUGUACAUGUUC | GAACAUGUACACAUAGUGC | Mouse | [136-154] ORF |
| 82 | UCAUCAGGAAAGACGACGU | ACGUCGUCUUUCCUGAUGA | | [864-882] ORF |
| 83 | CAACUAUGCAGGUGAGCCA | UGGCUCACCUGCAUAGUUG | | [742-760] ORF |
| 84 | GACAUACUUGAUGCCCAAA | UUUGGGCAUCAAGUAUGUC | | [481-499] ORF |
| 85 | AUCCAGAGAACAGGAUCAU | AUGAUCCUGUUCUCUGGAU | | [267-285] ORF |
| 86 | UGCCCAAAGAUGGCAAGAG | CUCUUGCCAUCUUUGGGCA | | [492-510] ORF |
| 87 | CCAGUGCAUUUAAGGCGCA | UGCGCCUUAAAUGCACUGG | | [14-32] 5′UTR |
| 88 | CCUGCAAAAGUCAGGGCAA | UUGCCCUGACUUUUGCAGG | | [907-925] ORF |
| 89 | GGGUCAUCAGGAAAGACGA | UCGUCUUUCCUGAUGACCC | | [861-879] ORF |
| 90 | CUGCAAAAGUCAGGGCAAG | CUUGCCCUGACUUUUGCAG | | [908-926] ORF |
| 91 | AGACCCUGAGCCCAACUAU | AUAGUUGGGCUCAGGGUCU | | [730-748] ORF |
| 92 | AGCCAGCACUAUGUGUACA | UGUACACAUAGUGCUGGCU | Mouse | [131-149] ORF |
| 93 | CUAUGCAGGUGAGCCAUAC | GUAUGGCUCACCUGCAUAG | | [745-763] ORF |
| 94 | UCAUUCACAAGCUCCUGGA | UCCAGGAGCUUGUGAAUGA | | [834-852] ORF |
| 95 | UGAGGUCAUUCACAAGCUC | GAGCUUGUGAAUGACCUCA | | [829-847] ORF |
| 96 | GACACCUUCAUCCGUCACA | UGUGACGGAUGAAGGUGUC | | [77-95] ORF |

TABLE N
21-mer oligomers for NOXO2 (NADPH oxidase organizer 2, NCF1)

| | | | | |
|---|---|---|---|---|
| | | | | Human-115298671 |
| Number | Sense siRNA | AntiSense siRNA | Other Sp | ORF: 71-1243 |

| | | | | |
|---|---|---|---|---|
| 1 | GCCUGUACAUACGUGUUCUAU | AUAGAACACGUAUGUACAGGC | | [1291-1311] 3′UTR |
| 2 | ACAUACUUGAUGCCCAAAGAU | AUCUUUGGGCAUCAAGUAUGU | | [482-502] ORF |
| 3 | UGCCUGUACAUACGUGUUCUA | UAGAACACGUAUGUACAGGCA | | [1290-1310] 3′UTR |
| 4 | CCCGUCCAUGUACCUGCAAAA | UUUUGCAGGUACAUGGACGGG | | [895-915] ORF |
| 5 | CAAAAAAGCCAGAGACAUACU | AGUAUGUCUCUGGCUUUUUUG | | [468-488] ORF |
| 6 | CCUGUACAUACGUGUUCUAUA | UAUAGAACACGUAUGUACAGG | | [1292-1312] 3′UTR |
| 7 | GGGCGAUCAAUCCAGAGAACA | UGUUCUCUGGAUUGAUCGCCC | | [258-278] ORF |
| 8 | GCCAUUGCCAACUACGAGAAG | CUUCUCGUAGUUGGCAAUGGC | | [557-577] ORF |
| 9 | UGUACAUACGUGUUCUAUAGA | UCUAUAGAACACGUAUGUACA | | [1294-1314] 3′UTR |
| 10 | CUGUACAUACGUGUUCUAUAG | CUAUAGAACACGUAUGUACAG | | [1293-1313] 3′UTR |
| 11 | AAAGCCAGAGACAUACUUGAU | AUCAAGUAUGUCUCUGGCUUU | | [472-492] ORF |
| 12 | GGCGAUCAAUCCAGAGAACAG | CUGUUCUCUGGAUUGAUCGCC | | [259-279] ORF |
| 13 | GUGUACAUGUUCCUGGUGAAA | UUUCACCAGGAACAUGUACAC | | [143-163] ORF |
| 14 | CCAUUGCCAACUACGAGAAGA | UCUUCUCGUAGUUGGCAAUGG | | [558-578] ORF |
| 15 | CCCACGGACAACCAGACAAAA | UUUUGUCUGGUUGUCCGUGGG | | [452-472] ORF |
| 16 | GUGCCUGUACAUACGUGUUCU | AGAACACGUAUGUACAGGCAC | | [1289-1309] 3′UTR |
| 17 | CCGUCCAUGUACCUGCAAAAG | CUUUUGCAGGUACAUGGACGG | | [896-916] ORF |
| 18 | CGGUUGGUGGUUCUGUCAGAU | AUCUGACAGAACCACCAACCG | | [643-663] ORF |
| 19 | UGUGUACAUGUUCCUGGUGAA | UUCACCAGGAACAUGUACACA | | [142-162] ORF |
| 20 | GGUGAAGCUGUUGAGGUCAUU | AAUGACCUCAACAGCUUCACC | | [818-838] ORF |
| 21 | CGAGUUCCAUAAAACCUUAAA | UUUAAGGUUUUAUGGAACUCG | | [214-234] ORF |
| 22 | CUACUUCCCGUCCAUGUACCU | AGGUACAUGGACGGGAAGUAG | | [889-909] ORF |
| 23 | CUGGUGGGUCAUCAGGAAAGA | UCUUUCCUGAUGACCCACCAG | | [856-876] ORF |
| 24 | UUGGUGGUUCUGUCAGAUGAA | UUCAUCUGACAGAACCACCAA | | [646-666] ORF |
| 25 | UCCCGUCCAUGUACCUGCAAA | UUUGCAGGUACAUGGACGGGA | | [894-914] ORF |
| 26 | GGGUGAAGCUGUUGAGGUCAU | AUGACCUCAACAGCUUCACCC | | [817-837] ORF |
| 27 | UGGUGGUUCUGUCAGAUGAAA | UUUCAUCUGACAGAACCACCA | | [647-667] ORF |
| 28 | GACUUCCUCUUUCCAGUGCAU | AUGCACUGGAAAGAGGAAGUC | | [2-22] 5′UTR |
| 29 | CUAUGUGUACAUGUUCCUGGU | ACCAGGAACAUGUACACAUAG | Rat, Mouse | [139-159] ORF |
| 30 | CAAGAGUACCGCGACAGACAU | AUGUCUGUCGCGGUACUCUUG | | [505-525] ORF |
| 31 | GGUUGGUGGUUCUGUCAGAUG | CAUCUGACAGAACCACCAACC | | [644-664] ORF |
| 32 | AGGGUGAAGCUGUUGAGGUCA | UGACCUCAACAGCUUCACCCU | | [816-836] ORF |
| 33 | CCAGACAAAAAAGCCAGAGAC | GUCUCUGGCUUUUUUGUCUGG | | [463-483] ORF |
| 34 | ACGAGUUCCAUAAAACCUUAA | UUAAGGUUUUAUGGAACUCGU | | [213-233] ORF |
| 35 | GAUCUACGAGUUCCAUAAAAC | GUUUUAUGGAACUCGUAGAUC | | [208-228] ORF |
| 36 | GUGAAGCUGUUGAGGUCAUUC | GAAUGACCUCAACAGCUUCAC | | [819-839] ORF |
| 37 | UGAGCCAUACGUCGCCAUCAA | UUGAUGGCGACGUAUGGCUCA | | [754-774] ORF |
| 38 | AGAGACAUACUUGAUGCCCAA | UUGGGCAUCAAGUAUGUCUCU | | [478-498] ORF |
| 39 | CUACGAGUUCCAUAAAACCUU | AAGGUUUUAUGGAACUCGUAG | | [211-231] ORF |
| 40 | ACUUCCUCUUUCCAGUGCAUU | AAUGCACUGGAAAGAGGAAGU | | [3-23] 5′UTR |
| 41 | CAGACAAAAAAGCCAGAGACA | UGUCUCUGGCUUUUUUGUCUG | | [464-484] ORF |
| 42 | CCACGGACAACCAGACAAAAA | UUUUUGUCUGGUUGUCCGUGG | | [453-473] ORF |
| 43 | UACGAGUUCCAUAAAACCUUA | UAAGGUUUUAUGGAACUCGUA | | [212-232] ORF |
| 44 | CCUUAAAAGAAAUGUUCCCUA | UAGGGAACAUUUCUUUUAAGG | | [228-248] ORF |
| 45 | GUACAUGUUCCUGGUGAAAUG | CAUUUCACCAGGAACAUGUAC | | [145-165] ORF |
| 46 | CGACUUCCUCUUUCCAGUGCA | UGCACUGGAAAGAGGAAGUCG | | [1-21] 5′UTR |
| 47 | CAACCAGACAAAAAAGCCAGA | UCUGGCUUUUUUGUCUGGUUG | | [460-480] ORF |
| 48 | ACGGACAACCAGACAAAAAAG | CUUUUUUGUCUGGUUGUCCGU | | [455-475] ORF |
| 49 | CACGGACAACCAGACAAAAAA | UUUUUUGUCUGGUUGUCCGUG | | [454-474] ORF |
| 50 | GGCUUUGAGAAGCGCUUCGUA | UACGAAGCGCUUCUCAAAGCC | | [107-127] ORF |
| 51 | CGCCAUUGCCAACUACGAGAA | UUCUCGUAGUUGGCAAUGGCG | | [556-576] ORF |
| 52 | GACAAAAAAGCCAGAGACAUA | UAUGUCUCUGGCUUUUUUGUC | | [466-486] ORF |
| 53 | GUUCUGUCAGAUGAAAGCAAA | UUUGCUUUCAUCUGACAGAAC | | [652-672] ORF |
| 54 | AGACAAAAAAGCCAGAGACAU | AUGUCUCUGGCUUUUUUGUCU | | [465-485] ORF |
| 55 | CCACCUCCUCGACUUCUUCAA | UUGAAGAAGUCGAGGAGGUGG | | [406-426] ORF |
| 56 | CCAACUAUGCAGGUGAGCCAU | AUGGCUCACCUGCAUAGUUGG | | [741-761] ORF |
| 57 | ACCAGACAAAAAAGCCAGAGA | UCUCUGGCUUUUUUGUCUGGU | | [462-482] ORF |
| 58 | UUCUUCAAGGUGCGCCCUGAU | AUCAGGGCGCACCUUGAAGAA | | [419-439] ORF |
| 59 | CGAUCAAUCCAGAGAACAGGA | UCCUGUUCUCUGGAUUGAUCG | | [261-281] ORF |
| 60 | UCCUCUUUCCAGUGCAUUUAA | UUAAAUGCACUGGAAAGAGGA | | [6-26] 5′UTR |
| 61 | CAGCCAGCACUAUGUGUACAU | AUGUACACAUAGUGCUGGCUG | Mouse | [130-150] ORF |
| 62 | GAAGACCCUGAGCCCAACUAU | AUAGUUGGGCUCAGGGUCUUC | | [728-748] ORF |
| 63 | GCCCAAAGAUGGCAAGAGUAC | GUACUCUUGCCAUCUUUGGGC | | [493-513] ORF |
| 64 | GACAUACUUGAUGCCCAAAGA | UCUUUGGGCAUCAAGUAUGUC | | [481-501] ORF |
| 65 | GAGACAUACUUGAUGCCCAAA | UUUGGGCAUCAAGUAUGUCUC | | [479-499] ORF |
| 66 | CUCCUCGACUUCUUCAAGGUG | CACCUUGAAGAAGUCGAGGAG | | [410-430] ORF |
| 67 | CAAUCCAGAGAACAGGAUCAU | AUGAUCCUGUUCUCUGGAUUG | | [265-285] ORF |
| 68 | CCGUGCCUGUACAUACGUGUU | AACACGUAUGUACAGGCACGG | | [1287-1307] 3′UTR |
| 69 | AGACGACGUCACAGGCUACUU | AAGUAGCCUGUGACGUCGUCU | | [874-894] ORF |
| 70 | GAAAGCAAAGCGAGGCUGGAU | AUCCAGCCUCGCUUUGCUUUC | | [664-684] ORF |
| 71 | AGAUGAAAGCAAAGCGAGGCU | AGCCUCGCUUUGCUUUCAUCU | | [660-680] ORF |
| 72 | GGUUCUGUCAGAUGAAAGCAA | UUGCUUUCAUCUGACAGAACC | | [651-671] ORF |
| 73 | GCACUAUGUGUACAUGUUCCU | AGGAACAUGUACACAUAGUGC | Mouse | [136-156] ORF |
| 74 | CAGCACUAUGUGUACAUGUUC | GAACAUGUACACAUAGUGCUG | Mouse | [134-154] ORF |
| 75 | CCAGCACUAUGUGUACAUGUU | AACAUGUACACAUAGUGCUGG | Mouse | [133-153] ORF |
| 76 | GAAAGACGACGUCACAGGCUA | UAGCCUGUGACGUCGUCUUUC | | [871-891] ORF |
| 77 | UGGUUCUGUCAGAUGAAAGCA | UGCUUUCAUCUGACAGAACCA | | [650-670] ORF |
| 78 | GGACAACCAGACAAAAAAGCC | GGCUUUUUUGUCUGGUUGUCC | | [457-477] ORF |
| 79 | CCUCCUCGACUUCUUCAAGGU | ACCUUGAAGAAGUCGAGGAGG | | [409-429] ORF |
| 80 | GCAAAAGUCAGGGCAAGACGU | ACGUCUUGCCCUGACUUUUGC | | [910-930] ORF |
| 81 | UGCCCAAAGAUGGCAAGAGUA | UACUCUUGCCAUCUUUGGGCA | | [492-512] ORF |
| 82 | CACCUCCUCGACUUCUUCAAG | CUUGAAGAAGUCGAGGAGGUG | | [407-427] ORF |
| 83 | AGGGCACACUUACCGAGUACU | AGUACUCGGUAAGUGUGCCCU | | [342-362] ORF |
| 84 | ACCUUAAAAGAAAUGUUCCCU | AGGGAACAUUUCUUUUAAGGU | | [227-247] ORF |
| 85 | CAACUAUGCAGGUGAGCCAUA | UAUGGCUCACCUGCAUAGUUG | | [742-762] ORF |
| 86 | GGACACCUUCAUCCGUCACAU | AUGUGACGGAUGAAGGUGUCC | | [76-96] ORF |
| 87 | AAAGAAAUGUUCCCUAUUGAG | CUCAAUAGGGAACAUUUCUUU | | [233-253] ORF |
| 88 | GCCAGCACUAUGUGUACAUGU | ACAUGUACACAUAGUGCUGGC | Mouse | [132-152] ORF |
| 89 | AGGCUACUUCCCGUCCAUGUA | UACAUGGACGGGAAGUAGCCU | | [886-906] ORF |
| 90 | GGUCAUCAGGAAAGACGACGU | ACGUCGUCUUUCCUGAUGACC | | [862-882] ORF |
| 91 | UGAGCCCAACUAUGCAGGUGA | UCACCUGCAUAGUUGGGCUCA | | [736-756] ORF |
| 92 | GGUGGUUCUGUCAGAUGAAAG | CUUUCAUCUGACAGAACCACC | | [648-668] ORF |
| 93 | CCAGAGACAUACUUGAUGCCC | GGGCAUCAAGUAUGUCUCUGG | | [476-496] ORF |
| 94 | GCCAGAGACAUACUUGAUGCC | GGCAUCAAGUAUGUCUCUGGC | | [475-495] ORF |
| 95 | CCUCUUUCCAGUGCAUUUAAG | CUUAAAUGCACUGGAAAGAGG | | [7-27] 5′UTR |
| 96 | UCCAUGUACCUGCAAAAGUCA | UGACUUUUGCAGGUACAUGGA | | [899-919] ORF |
| 97 | CCUGCAAAAGUCAGGGCAAGA | UCUUGCCCUGACUUUUGCAGG | | [907-927] ORF |
| 98 | CCAUGUACCUGCAAAAGUCAG | CUGACUUUUGCAGGUACAUGG | | [900-920] ORF |
| 99 | GUGGGUCAUCAGGAAAGACGA | UCGUCUUUCCUGAUGACCCAC | | [859-879] ORF |
| 100 | GCUGUUGAGGUCAUUCACAAG | CUUGUGAAUGACCUCAACAGC | | [824-844] ORF |
| 101 | AGCCAGCACUAUGUGUACAUG | CAUGUACACAUAGUGCUGGCU | Mouse | [131-151] ORF |
| 102 | CCAGCCAGCACUAUGUGUACA | UGUACACAUAGUGCUGGCUGG | Mouse | [129-149] ORF |
| 103 | GGUCAUUCACAAGCUCCUGGA | UCCAGGAGCUUGUGAAUGACC | | [832-852] ORF |
| 104 | AGCUGUUGAGGUCAUUCACAA | UUGUGAAUGACCUCAACAGCU | | [823-843] ORF |
| 105 | GAAGCUGUUGAGGUCAUUCAC | GUGAAUGACCUCAACAGCUUC | | [821-841] ORF |
| 106 | GUCAUUCACAAGCUCCUGGAC | GUCCAGGAGCUUGUGAAUGAC | | [833-853] ORF |
| 107 | GACACCUUCAUCCGUCACAUC | GAUGUGACGGAUGAAGGUGUC | | [77-97] ORF |
| 108 | GAGGUCAUUCACAAGCUCCUG | CAGGAGCUUGUGAAUGACCUC | | [830-850] ORF |

TABLE O
19-mer oligomers for NOXA1

| | | | | |
|---|---|---|---|---|
| Number | Sense siRNA | AntiSense siRNA | Other Sp | Human-41393186 ORF: 136-1587 |

| | | | | |
|---|---|---|---|---|
| 1 | GCCCUAAUGAUGCUGUGUC | GACACAGCAUCAUUAGGGC | | [1581-1599] ORF + 3′UTR |
| 2 | GGAGAUCAGCCCUAAUGAU | AUCAUUAGGGCUGAUCUCC | | [1573-1591] ORF + 3′UTR |
| 3 | GGGAGGUGCUACACAAUGU | ACAUUGUGUAGCACCUCCC | Chimpanzee | [500-518] ORF |
| 4 | AGGAGGCUCUGUCUGACUU | AAGUCAGACAGAGCCUCCU | | [401-419] ORF |
| 5 | CUAAUGAUGCUGUGUCCAU | AUGGACACAGCAUCAUUAG | | [1584-1602] ORF + 3′UTR |
| 6 | CCCUAAUGAUGCUGUGUCC | GGACACAGCAUCAUUAGGG | | [1582-1600] ORF + 3′UTR |
| 7 | GGAGCAAGUUGGCAAACAG | CUGUUUGCCAACUUGCUCC | Chimpanzee | [951-969] ORF |
| 8 | GGAGGUGCUACACAAUGUG | CACAUUGUGUAGCACCUCC | Chimpanzee | [501-519] ORF |
| 9 | AUGAUGCUGUGUCCAUGAU | AUCAUGGACACAGCAUCAU | | [1587-1605] 3′UTR |
| 10 | GAGGCUCUGUCUGACUUCU | AGAAGUCAGACAGAGCCUC | | [403-421] ORF |
| 11 | CUGGGAGGUGCUACACAAU | AUUGUGUAGCACCUCCCAG | | [498-516] ORF |
| 12 | UGGAGCAAGUUGGCAAACA | UGUUUGCCAACUUGCUCCA | Chimpanzee | [950-968] ORF |
| 13 | GGUGGAGCAAGUUGGCAAA | UUUGCCAACUUGCUCCACC | Chimpanzee | [948-966] ORF |
| 14 | GUGUCCAUGAUGCUUUUAA | UUAAAAGCAUCAUGGACAC | Chimpanzee | [1595-1613] 3′UTR |
| 15 | UGUCCAUGAUGCUUUUAAU | AUUAAAAGCAUCAUGGACA | Chimpanzee | [1596-1614] 3′UTR |
| 16 | GUCCAUGAUGCUUUUAAUA | UAUUAAAAGCAUCAUGGAC | Chimpanzee | [1597-1615] 3′UTR |
| 17 | AGGUGGAGCAAGUUGGCAA | UUGCCAACUUGCUCCACCU | Chimpanzee | [947-965] ORF |
| 18 | CUGUGUCCAUGAUGCUUUU | AAAAGCAUCAUGGACACAG | Chimpanzee | [1593-1611] 3′UTR |
| 19 | GCUGUGUCCAUGAUGCUUU | AAAGCAUCAUGGACACAGC | Chimpanzee | [1592-1610] 3′UTR |
| 20 | UGACCAAGGACACCUGCAU | AUGCAGGUGUCCUUGGUCA | Rat | [329-347] ORF |
| 21 | GCAUUUGACCAAGCCGUGA | UCACGGCUUGGUCAAAUGC | | [313-331] ORF |
| 22 | CAGCCCUAAUGAUGCUGUG | CACAGCAUCAUUAGGGCUG | | [1579-1597] ORF + 3′UTR |
| 23 | UCAGCCCUAAUGAUGCUGU | ACAGCAUCAUUAGGGCUGA | | [1578-1596] ORF + 3′UTR |
| 24 | GGGAGAUCAGCCCUAAUGA | UCAUUAGGGCUGAUCUCCC | | [1572-1590] ORF + 3′UTR |
| 25 | ACUUCCAGCUGGCAAGGUU | AACCUUGCCAGCUGGAAGU | | [380-398] ORF |
| 26 | AGGGAGAUCAGCCCUAAUG | CAUUAGGGCUGAUCUCCCU | | [1571-1589] ORF + 3′UTR |
| 27 | UGGGCAACUCAGUUACCUA | UAGGUAACUGAGUUGCCCA | Chimpanzee | [1188-1206] ORF |
| 28 | GGGCAACUCAGUUACCUAG | CUAGGUAACUGAGUUGCCC | Chimpanzee | [1189-1207] ORF |
| 29 | AGCUUGGGCAACUCAGUUA | UAACUGAGUUGCCCAAGCU | Chimpanzee | [1184-1202] ORF |
| 30 | CCAGGUCCCUAAUCAUGGA | UCCAUGAUUAGGGACCUGG | Chimpanzee | [830-848] ORF |
| 31 | CAGGGAGAUCAGCCCUAAU | AUUAGGGCUGAUCUCCCUG | | [1570-1588] ORF + 3′UTR |
| 32 | GCAACUCAGUUACCUAGCC | GGCUAGGUAACUGAGUUGC | Chimpanzee | [1191-1209] ORF |
| 33 | CAUGAUGCCAGGUCCCUAA | UUAGGGACCUGGCAUCAUG | Chimpanzee | [823-841] ORF |
| 34 | UGCUACACAAUGUGGCGUC | GACGCCACAUUGUGUAGCA | Chimpanzee | [506-524] ORF |
| 35 | GCUUGGGCAACUCAGUUAC | GUAACUGAGUUGCCCAAGC | Chimpanzee | [1185-1203] ORF |
| 36 | GCAAGUUGGCAAACAGGCU | AGCCUGUUUGCCAACUUGC | Chimpanzee | [954-972] ORF |
| 37 | GGUCCCUAAUCAUGGACUC | GAGUCCAUGAUUAGGGACC | Chimpanzee | [833-851] ORF |
| 38 | AGGUCCCUAAUCAUGGACU | AGUCCAUGAUUAGGGACCU | Chimpanzee | [832-850] ORF |

TABLE P
21-mer oligomers for NOXA1

| | | | | |
|---|---|---|---|---|
| | | | | Human-41393185 |
| Number | Sense siRNA | AntiSense siRNA | Other Sp | ORF: 136-1587 |

| | | | | |
|---|---|---|---|---|
| 1 | CCCUAAUGAUGCUGUGUCCAU | AUGGACAGAGCAUCAUUAGGG | | [1582-1602] ORF + 3′UTR |
| 2 | CUAAUGAUGCUGUGUCCAUGA | UCAUGGACACAGCAUCAUUAG | | [1584-1604] ORF + 3′UTR |
| 3 | GCCCUAAUGAUGCUGUGUCCA | UGGACACAGCAUCAUUAGGGC | | [1581-1601] ORF + 3′UTR |
| 4 | UGGGAGGUGCUACACAAUGUG | CACAUUGUGUAGCACCUCCCA | Chimpanzee | [499-519] ORF |
| 5 | GUGUCCAUGAUGCUUUUAAUA | UAUUAAAAGCAUCAUGGACAC | Chimpanzee | [1595-1615] 3′UTR |
| 6 | GUGGAGCAAGUUGGCAAACAG | CUGUUUGCCAACUUGCUCCAC | Chimpanzee | [949-969] ORF |
| 7 | CAGGUGGAGCAAGUUGGCAAA | UUUGCCAACUUGCUCCACCUG | Chimpanzee | [946-966] ORF |
| 8 | UGUGUCCAUGAUGCUUUUAAU | AUUAAAAGCAUCAUGGACACA | Chimpanzee | [1594-1614] 3′UTR |
| 9 | GCUGUGUCCAUGAUGCUUUUA | UAAAAGCAUCAUGGACACAGC | Chimpanzee | [1592-1612] 3′UTR |
| 10 | CUGUGUCCAUGAUGCUUUUAA | UUAAAAGCAUCAUGGACACAG | Chimpanzee | [1593-1613] 3′UTR |
| 11 | CCAUGAUGCCAGGUCCCUAAU | AUUAGGGACCUGGCAUCAUGG | Chimpanzee | [822-842] ORF |
| 12 | AGCAGGGAGAUCAGCCCUAAU | AUUAGGGCUGAUCUCCCUGCU | | [1568-1588] ORF + 3′UTR |
| 13 | CAACUUCCAGCUGGCAAGGUU | AACCUUGCCAGCUGGAAGUUG | | [378-398] ORF |
| 14 | CCAGCUUGGGCAACUCAGUUA | UAACUGAGUUGCCCAAGCUGG | Chimpanzee | [1182-1202] ORF |
| 15 | CUUGGGCAACUCAGUUACCUA | UAGGUAACUGAGUUGCCCAAG | Chimpanzee | [1186-1206] ORF |
| 16 | UGAUGCCAGGUCCCUAAUCAU | AUGAUUAGGGACCUGGCAUCA | Chimpanzee | [825-845] ORF |
| 17 | UCAGCCCUAAUGAUGCUGUGU | ACACAGCAUCAUUAGGGCUGA | | [1578-1598] ORF + 3′UTR |
| 18 | GGGAGAUCAGCCCUAAUGAUG | CAUCAUUAGGGCUGAUCUCCC | | [1572-1592] ORF + 3′UTR |
| 19 | AGGGAGAUCAGCCCUAAUGAU | AUCAUUAGGGCUGAUCUCCCU | | [1571-1591] ORF + 3′UTR |
| 20 | CAGGGAGAUCAGCCCUAAUGA | UCAUUAGGGCUGAUCUCCCUG | | [1570-1590] ORF + 3′UTR |
| 21 | CAGGUCCCUAAUCAUGGACUC | GAGUCCAUGAUUAGGGACCUG | Chimpanzee | [831-851] ORF |
| 22 | CCAGGUCCCUAAUCAUGGACU | AGUCCAUGAUUAGGGACCUGG | Chimpanzee | [830-850] ORF |
| 23 | UGCUGUGUCCAUGAUGCUUUU | AAAAGCAUCAUGGACACAGCA | Chimpanzee | [1591-1611] 3′UTR |
| 24 | CAGCUUGGGCAACUCAGUUAC | GUAACUGAGUUGCCCAAGCUG | Chimpanzee | [1183-1203] ORF |
| 25 | UGAUGCUGUGUCCAUGAUGCU | AGCAUCAUGGACACAGCAUCA | | [1588-1608] 3′UTR |
| 26 | ACCAUGAUGCCAGGUCCCUAA | UUAGGGACCUGGCAUCAUGGU | Chimpanzee | [821-841] ORF |

TABLE Q
19-mer oligomers for NOXA2 (NCF2, neutrophil cytosolic factor 2, p67phox)

| | | | | |
|---|---|---|---|---|
| Number | Sense siRNA | AntiSense siRNA | Other Sp | Human-67189969 ORF: 253-1633 |

| | | | | |
|---|---|---|---|---|
| 1 | CCCUGCAACUACCUUGAAC | GUUCAAGGUAGUUGCAGGG | Guinea-pig | [1120-1138] ORF |
| 2 | GGAAAGUGAAAAAGCUGAU | AUCAGCUUUUUCACUUUCC | | [1572-1590] ORF |
| 3 | GUGAAAAACUACUGCCUGA | UCAGGCAGUAGUUUUUCAC | Rat, Mouse | [1501-1519] ORF |
| 4 | GCAUUUGCAACACUGAGAA | UUCUCAGUGUUGCAAAUGC | | [79-97] 5′UTR |
| 5 | CCUUAUAGGUUACUCAUAA | UUAUGAGUAACCUAUAAGG | | [2334-2352] 3′UTR |
| 6 | CCAAGGAGUAAGUACAAAU | AUUUGUACUUACUCCUUGG | | [2265-2283] 3′UTR |
| 7 | UCAAAGGUGAAUGAAGAAU | AUUCUUCAUUCACCUUUGA | | [1714-1732] ORF |
| 8 | ACCCAAGGAAAGUGAAAAA | UUUUUCACUUUCCUUGGGU | Guinea-pig | [1566-1584] ORF |
| 9 | GAAAAACUACUGCCUGACU | AGUCAGGCAGUAGUUUUUC | Mouse | [1503-1521] ORF |
| 10 | CCUUAAAGAAGCCUUGAUU | AAUCAAGGCUUCUUUAAGG | | [531-549] ORF |
| 11 | CGCAACUACAGAUUUGGAA | UUCCAAAUCUGUAGUUGCG | | [1794-1812] ORF |
| 12 | CCAAAGUUUUUGUUGAAGA | UCUUCAACAAAAACUUUGG | | [1772-1790] ORF |
| 13 | GCUUUCCAGAUGAACCCAA | UUGGGUUCAUCUGGAAAGC | | [1553-1571] ORF |
| 14 | AGGUGAAAAACUACUGCCU | AGGCAGUAGUUUUUCACCU | | [1499-1517] ORF |
| 15 | UCCCUGCAACUACCUUGAA | UUCAAGGUAGUUGCAGGGA | Guinea-pig | [1119-1137] ORF |
| 16 | UCAUGUUCAACGGGCAGAA | UUCUGCCCGUUGAACAUGA | | [1091-1109] ORF |
| 17 | ACCUUAAAGAAGCCUUGAU | AUCAAGGCUUCUUUAAGGU | | [530-548] ORF |
| 18 | CCAGACAGAGAAAUAUGAU | AUCAUAUUUCUCUGUCUGG | | [498-516] ORF |
| 19 | UGUACACUAUCCUGAAGAA | UUCUUCAGGAUAGUGUACA | | [389-407] ORF |
| 20 | GGUGUUAUCAAAGGUGAAU | AUUCACCUUUGAUAACACC | | [1707-1725] ORF |
| 21 | UGAUGCUAAUAACCAGACA | UGUCUGGUUAUUAGCAUCA | | [1587-1605] ORF |
| 22 | CCCAAGGAAAGUGAAAAAG | CUUUUUCACUUUCCUUGGG | | [1567-1585] ORF |
| 23 | GGCCAGGUGAAAAACUACU | AGUAGUUUUUCACCUGGCC | | [1495-1513] ORF |
| 24 | ACACUGAGAAGUUAUCUUA | UAAGAUAACUUCUCAGUGU | | [88-106] 5′UTR |
| 25 | ACAUUGUCUUUGUCUUGAA | UUCAAGACAAAGACAAUGU | | [1046-1064] ORF |
| 26 | UCGACCAAAUGAGAGACAA | UUGUCUCUCAUUUGGUCGA | | [801-819] ORF |
| 27 | GGCUGCAUGUACACUAUCC | GGAUAGUGUACAUGCAGCC | | [382-400] ORF |
| 28 | CGGACAAGAAGGACUGGAA | UUCCAGUCCUUCUUGUCCG | | [302-320] ORF |
| 29 | GGUUACUCAUAAGGCAUAU | AUAUGCCUUAUGAGUAACC | | [2341-2359] 3′UTR |
| 30 | UCACACUGUAAACAUUCAU | AUGAAUGUUUACAGUGUGA | | [2157-2175] 3′UTR |
| 31 | CUAGACAAUGAUGUGAGUA | UACUCACAUCAUUGUCUAG | | [1975-1993] 3′UTR |
| 32 | AGAAAAUGAAGCCCUAUUA | UAAUAGGGCUUCAUUUUCU | | [1859-1877] 3′UTR |
| 33 | CCCAAAGUUUUUGUUGAAG | CUUCAACAAAAACUUUGGG | | [1771-1789] ORF |
| 34 | GGAGGCACUCUUCAGUUAU | AUAACUGAAGAGUGCCUCC | | [1638-1656] ORF |
| 35 | GAAGACAGCAUGAAGGAUG | CAUCCUUCAUGCUGUCUUC | | [1471-1489] ORF |
| 36 | CACUCAAGGUGCACUACAA | UUGUAGUGCACCUUGAGUG | Guinea-pig | [1310-1328] ORF |
| 37 | AGACAAAAGAAGAGCUCCA | UGGAGCUCUUCUUUUGUCU | | [1013-1031] ORF |
| 38 | CGACCAAAUGAGAGACAAG | CUUGUCUCUCAUUUGGUCG | | [802-820] ORF |
| 39 | UGAUUCAGCUUCGAGGGAA | UUCCCUCGAAGCUGAAUCA | | [545-563] ORF |
| 40 | CAGACAGAGAAAUAUGAUU | AAUCAUAUUUCUCUGUCUG | | [499-517] ORF |
| 41 | GAAGCAUUAACCGAGACAA | UUGUCUCGGUUAAUGCUUC | | [437-455] ORF |
| 42 | GCUGCAUGUACACUAUCCU | AGGAUAGUGUACAUGCAGC | | [383-401] ORF |
| 43 | AGGUUACUCAUAAGGCAUA | UAUGCCUUAUGAGUAACCU | | [2340-2358] 3′UTR |
| 44 | UGAUGUGAGUACCCAGGAU | AUCCUGGGUACUCACAUCA | | [1963-2001] 3′UTR |
| 45 | GUAUACUGUACUGAGACAU | AUGUCUCAGUACAGUAUAC | | [1908-1926] 3′UTR |
| 46 | GAUGCUAAUAACCAGACAA | UUGUCUGGUUAUUAGCAUC | | [1588-1606] ORF |
| 47 | GCCUGAGACAAAAGAAGAG | CUCUUCUUUUGUCUCAGGC | | [1008-1028] ORF |
| 48 | AGAGAAAUAUGAUUUGGCU | AGCCAAAUCAUAUUUCUCU | | [504-522] ORF |
| 49 | CCAGAAGCAUUAACCGAGA | UCUCGGUUAAUGCUUCUGG | | [434-452] ORF |
| 50 | GGCCUCCUAGUUUCUACCU | AGGUAGAAACUAGGAGGCC | | [230-248] 5′UTR |
| 51 | CAUUCAUCAAGCUGUACAU | AUGUACAGCUUGAUGAAUG | | [2169-2187] 3′UTR |
| 52 | ACACUGUAAACAUUCAUCA | UGAUGAAUGUUUACAGUGU | | [2159-2177] 3′UTR |
| 53 | ACAAUGAUGUGAGUACCCA | UGGGUACUCACAUCAUUGU | | [1979-1997] 3′UTR |
| 54 | UAACCUACUAGACAAUGAU | AUCAUUGUCUAGUAGGUUA | | [1988-1986] 3′UTR |
| 55 | GGAUGUUUCACAAACUACA | UGUAGUUUGUGAAACAUCC | | [1833-1851] 3′UTR |
| 56 | UCGGAGAGAAGUCUAGGAU | AUCCUAGACUUCUCUCCGA | | [1818-1836] ORF + 3′UTR |
| 57 | AGGCACUCUUCAGUUAUGA | UCAUAACUGAAGAGUGCCU | | [1640-1658] ORF |
| 58 | CAAGGAAAGUGAAAAAGCU | AGCUUUUUCACUUUCCUUG | | [1569-1587] ORF |
| 59 | UGAACCCAAGGAAAGUGAA | UUCACUUUCCUUGGGUUCA | | [1563-1581] ORF |
| 60 | ACAAAAAGAAGAGCCUAAG | CUUAGGCUCUUCUUUUUGU | | [1260-1278] ORF |
| 61 | AACAAAAAGAAGAGCCUAA | UUAGGCUCUUCUUUUUGUU | | [1259-1277] ORF |
| 62 | AGGCCAGAAACAAAAAGAA | UUCUUUUUGUUUCUGGCCU | | [1251-1269] ORF |
| 63 | AGCUCCUCCUAGUUCCAAA | UUUGGAACUAGGAGGAGCU | | [1206-1224] ORF |
| 64 | CAAAAGAAGAGCUCCAGGU | ACCUGGAGCUCUUCUUUUG | | [1016-1034] ORF |
| 65 | CCGUGUGCUAUUUGGGUUU | AAACCCAAAUAGCACACGG | | [967-1005] ORF |
| 66 | UGGUGGAUCAAGACAGUUU | AAACUGUCUUGAUCCACCA | | [875-893] ORF |
| 67 | GGAAUGGAAAAAAGCUGAA | UUCAGCUUUUUUCCAUUCC | | [657-675] ORF |
| 68 | CUCUCUGCUUCUUUCCUUU | AAAGGAAAGAAGCAGAGAG | | [4-22] 5′UTR |
| 69 | GACAGAGAAAUAUGAUUUG | CAAAUCAUAUUUCUCUGUC | | [501-519] ORF |
| 70 | CUACCAGACAGAGAAAUAU | AUAUUUCUCUGUCUGGUAG | | [495-513] ORF |
| 71 | GCAUUAACCGAGACAAGCA | UGCUUGUCUCGGUUAAUGC | | [440-458] ORF |
| 72 | UGCAUGUACACUAUCCUGA | UCAGGAUAGUGUACAUGCA | | [385-403] ORF |
| 73 | ACAAAAACAAGACACCCAA | UUGGGUGUCUUGUUUUUGU | | [2238-2258] 3′UTR |
| 74 | ACAUUCAUCAAGCUGUACA | UGUACAGCUUGAUGAAUGU | | [2158-2186] 3′UTR |
| 75 | CACUGUAAACAUUCAUCAA | UUGAUGAAUGUUUACAGUG | | [2160-2178] 3′UTR |
| 76 | CUACUAGACAAUGAUGUGA | UCACAUCAUUGUCUAGUAG | | [1972-1990] 3′UTR |
| 77 | ACAAAGCUGAAGAAAAUGA | UCAUUUUCUUCAGCUUUGU | | [1849-1867] 3′UTR |
| 78 | CAAAGUUUUUGUUGAAGAC | GUCUUCAACAAAAACUUUG | | [1773-1791] ORF |
| 79 | GUGUUAUCAAAGGUGAAUG | CAUUCACCUUUGAUAACAC | | [1708-1726] ORF |
| 80 | CUCUUCAGUUAUGAGGCUA | UAGCCUCAUAACUGAAGAG | | [1645-1663] ORF |
| 81 | GGCUGGAACACACUAAGCU | AGCUUAGUGUGUUCCAGCC | | [1409-1427] ORF |
| 82 | CACUGAGAAGUUAUCUUAA | UUAAGAUAACUUCUCAGUG | | [89-107] 5′UTR |
| 83 | GCCUAAGGAAGUGAAGCUC | GAGCUUCACUUCCUUAGGC | | [1272-1290] ORF |
| 84 | GAAGAGCCUAAGGAAGUGA | UCACUUCCUUAGGCUCUUC | Rat | [1267-1285] ORF |
| 85 | AGAAGAGCCUAAGGAAGUG | CACUUCCUUAGGCUCUUCU | Rat | [1265-1284] ORF |
| 86 | CCAGGCCAGAAACAAAAAG | CUUUUUGUUUCUGGCCUGG | | [1249-1267] ORF |
| 87 | GACAAAAGAAGAGCUCCAG | CUGGAGCUCUUCUUUUGUC | | [1014-1032] ORF |
| 88 | GGCAUCUGUGGUGGAUCAA | UUGAUCCACCACAGAUGCC | Guinea-pig | [867-885] ORF |
| 89 | GACCAAAUGAGAGACAAGU | ACUUGUCUCUCAUUUGGUC | | [803-821] ORF |
| 90 | GCUGUUUCGACCAAAUGAG | CUCAUUUGGUCGAAACAGC | | [795-813] ORF |
| 91 | GACAUUCCAAAAUCGACAA | UUGUCGAUUUUGGAAUGUC | | [716-734] ORF |
| 92 | UCUCUCUGCUUCUUUCCUU | AAGGAAAGAAGCAGAGAGA | | [3-21] 5′UTR |
| 93 | CUCAUAAGGCAUAUACAAA | UUUGUAUAUGCCUUAUGAG | | [2346-2364] 3′UTR |
| 94 | GUUACUCAUAAGGCAUAUA | UAUAUGCCUUAUGAGUAAC | | [2342-2360] 3′UTR |
| 95 | CACACUGUAAACAUUCAUC | GAUGAAUGUUUACAGUGUG | | [2156-2176] 3′UTR |
| 96 | ACCUACUAGACAAUGAUGU | ACAUCAUUGUCUAGUAGGU | | [1970-1988] 3′UTR |
| 97 | GUCUAGGAUGUUUCACAAA | UUUGUGAAACAUCCUAGAC | | [1828-1846] ORF + 3′UTR |
| 98 | AGUCUAGGAUGUUUCACAA | UUGUGAAACAUCCUAGACU | | [1827-1845] ORF + 3′UTR |
| 99 | CAACUACAGAUUUGGAAAG | CUUUCCAAAUCUGUAGUUG | | [1796-1814] ORF |
| 100 | UGAAUGAAGAAUGGCUGGA | UCCAGCCAUUCUUCAUUCA | Rat, Mouse | [1721-1739] ORF |
| 101 | GGUGAAUGAAGAAUGGCUG | CAGCCAUUCUUCAUUCACC | | [1719-1737] ORF |
| 102 | AGGUGAAUGAAGAAUGGCU | AGCCAUUCUUCAUUCACCU | | [1718-1736] ORF |
| 103 | CCAGACAACAGAACCUCAG | CUGAGGUUCUGUUGUCUGG | | [1599-1617] ORF |
| 104 | GAAAGUGAAAAAGCUGAUG | CAUCAGCUUUUUCACUUUC | | [1573-1591] ORF |
| 105 | GGGCCAGGUGAAAAACUAC | GUAGUUUUUCACCUGGCCC | | [1494-1512] ORF |
| 106 | GGCCAGAAACAAAAAGAAG | CUUCUUUUUGUUUCUGGCC | | [1252-1270] ORF |
| 107 | GCAAUGAUAACUGGGCCAC | GUGGCCCAGUUAUCAUUGC | | [1070-1088] ORF |
| 108 | ACAUUCCAAAAUCGACAAG | CUUGUCGAUUUUGGAAUGU | | [717-735] ORF |
| 109 | AGCUGAUAGACUAUAAGAU | AUCUUAUAGUCUAUCAGCU | | [566-584] ORF |
| 110 | CUAUCAAAGACCUUAAAGA | UCUUUAAGGUCUUUGAUAG | Mouse | [521-539] ORF |
| 111 | GAGAAAUAUGAUUUGGCUA | UAGCCAAAUCAUAUUUCUC | | [505-523] ORF |
| 112 | ACCAGACAGAGAAAUAUGA | UCAUAUUUCUCUGUCUGGU | | [497-515] ORF |
| 113 | AGAAGCAUUAACCGAGACA | UGUCUCGGUUAAUGCUUCU | | [436-454] ORF |
| 114 | ACAUGACUGAAGCAGAGAA | UUCUCUGCUUCAGUCAUGU | | [407-425] ORF |
| 115 | CCUUUUCUCUCUCAUGGUA | UACCAUGAGAGAGAAAAGG | | [18-36] 5′UTR |
| 116 | ACCUAAUCAUGUCCCUGGU | ACCAGGGACAUGAUUAGGU | | [245-263] 5′UTR + ORF |
| 117 | ACUGUAAACAUUCAUCAAG | CUUGAUGAAUGUUUACAGU | | [2161-2179] 3′UTR |
| 118 | GCCCUAUUACUUGUUUGUA | UACAAACAAGUAAUAGGGC | | [1869-1887] 3′UTR |
| 119 | GCGCAACUACAGAUUUGGA | UCCAAAUCUGUAGUUGCGC | | [1793-1811] ORF |
| 120 | UGGUGUUAUCAAAGGUGAA | UUCACCUUUGAUAACACCA | | [1706-1724] ORF |
| 121 | CUGGUGUUAUCAAAGGUGA | UCACCUUUGAUAACACCAG | | [1705-1723] ORF |
| 122 | CCUGGUGUUAUCAAAGGUG | CACCUUUGAUAACACCAGG | | [1704-1722] ORF |
| 123 | UAAUCCUGGUGUUAUCAAA | UUUGAUAACACCAGGAUUA | | [1700-1718] ORF |
| 124 | AGGAAAGUGAAAAAGCUGA | UCAGCUUUUUCACUUUCCU | | [1571-1589] ORF |
| 125 | CCAAGGAAAGUGAAAAAGC | GCUUUUUCACUUUCCUUGG | | [1568-1586] ORF |
| 126 | GAACACACUAAGCUGAGCU | AGCUCAGCUUAGUGUGUUC | | [1414-1432] ORF |
| 127 | GGAACACACUAAGCUGAGC | GCUCAGCUUAGUGUGUUCC | | [1413-1431] ORF |
| 128 | ACUGAGAAGUUAUCUUAAG | CUUAAGAUAACUUCUCAGU | | [90-108] 5′UTR |
| 129 | AACACUGAGAAGUUAUCUU | AAGAUAACUUCUCAGUGUU | | [87-105] 5′UTR |
| 130 | UUGUCUUUGUCUUGAAGAA | UUCUUCAAGACAAAGACAA | | [1049-1067] ORF |
| 131 | CUGAGACAAAAGAAGAGCU | AGCUCUUCUUUUGUCUCAG | | [1010-1028] ORF |
| 132 | GGUGGAUCAAGACAGUUUC | GAAACUGUCUUGAUCCACC | | [876-894] ORF |
| 133 | GCAUCUGUGGUGGAUCAAG | CUUGAUCCACCACAGAUGC | Guinea-pig | [868-886] ORF |
| 134 | CGAUGGAGUGUGUCUGGAA | UUCCAGACACACUCCAUCG | | [737-755] ORF |
| 135 | GGGAACCAGCUGAUAGACU | AGUCUAUCAGCUGGUUCCC | Mouse | [559-577] ORF |
| 136 | ACAGAGAAAUAUGAUUUGG | CCAAAUCAUAUUUCUCUGU | | [502-520] ORF |
| 137 | CUACUACCAGACAGAGAAA | UUUCUCUGUCUGGUAGUAG | | [492-510] ORF |
| 138 | CUGAAGAACAUGACUGAAG | CUUCAGUCAUGUUCUUCAG | | [400-418] ORF |
| 139 | UCCUGAAGAACAUGACUGA | UCAGUCAUGUUCUUCAGGA | | [398-416] ORF |
| 140 | GGACAAGAAGGACUGGAAG | CUUCCAGUCCUUCUUGUCC | | [303-321] ORF |
| 141 | UUACUCAUAAGGCAUAUAC | GUAUAUGCCUUAUGAGUAA | | [2343-2361] 3′UTR |
| 142 | GCUUGAAGUAACCUUAUAG | CUAUAAGGUUACUUCAAGC | | [2323-2341] 3′UTR |
| 143 | UACUGUAAUUGGCUCUUAA | UUAAGAGCCAAUUACAGUA | | [2303-2321] 3′UTR |
| 144 | UUACUGUAAUUGGCUCUUA | UAAGAGCCAAUUACAGUAA | | [2302-2320] 3′UTR |
| 145 | UCCUGUUUCUGAACCAUUA | UAAUGGUUCAGAAACAGGA | | [2286-2304] 3′UTR |
| 146 | GUAAGUACAAAUAUUCCUG | CAGGAAUAUUUGUACUUAC | | [2272-2290] 3′UTR |
| 147 | AAACCUAGCAUCUUACAAA | UUUGUAAGAUGCUAGGUUU | | [2224-2242] 3′UTR |
| 148 | UCAAGCUGUACAUUUGGUG | CACCAAAUGUACAGCUUGA | | [2175-2193] 3′UTR |
| 149 | CUGUAAACAUUCAUCAAGC | GCUUGAUGAAUGUUUACAG | | [2162-2180] 3′UTR |
| 150 | CCUGUUUCUUGCUCUGAGU | ACUCAGAGCAAGAAACAGG | | [2119-2137] 3′UTR |
| 151 | AGAGAGUCUUCCAGGUACU | AGUACCUGGAAGACUCUCU | | [2097-2115] 3′UTR |
| 152 | GAAAAGUGGAUGGAAGUGU | ACACUUCCAUCCACUUUUC | | [2064-2082] 3′UTR |
| 153 | UGAGACAUUACAGUUUGGA | UCCAAACUGUAAUGUCUCA | | [1919-1937] 3′UTR |
| 154 | CUGCUGUAUACUGUACUGA | UCAGUACAGUAUACAGCAG | | [1903-1921] 3′UTR |
| 155 | CCCUUCUGCUGUAUACUGU | ACAGUAUACAGCAGAAGGG | | [1898-1916] 3′UTR |
| 156 | AAGAAAAUGAAGCCCUAUU | AAUAGGGCUUCAUUUUCUU | | [1858-1876] 3′UTR |
| 157 | CGGAGAGAAGUCUAGGAUG | CAUCCUAGACUUCUCUCCG | | [1819-1837] ORF + 3′UTR |
| 158 | CUACAGAUUUGGAAAGCAC | GUGCUUUCCAAAUCUGUAG | | [1799-1817] ORF |
| 159 | GUUGAAGACUGCGCAACUA | UAGUUGCGCAGUCUUCAAC | | [1783-1801] ORF |
| 160 | GACAACAGAACCUCAGCUU | AAGCUGAGGUUCUGUUGUC | | [1602-1620] ORF |
| 161 | ACACACUAAGCUGAGCUAU | AUAGCUCAGCUUAGUGUGU | | [1416-1434] ORF |
| 162 | AACACACUAAGCUGAGCUA | UAGCUCAGCUUAGUGUGUU | | [1415-1433] ORF |
| 163 | GAGAAGUUAUCUUAAGGGA | UCCCUUAAGAUAACUUCUC | | [93-111] 5′UTR |
| 164 | AGGAAGUGAAGCUCAGUGU | ACACUGAGCUUCACUUCCU | | [1277-1295] ORF |
| 165 | CACCAGGCCAGAAACAAAA | UUUUGUUUCUGGCCUGGUG | | [1247-1265] ORF |
| 166 | GGGAACAUUGUCUUUGUCU | AGACAAAGACAAUGUUCCC | | [1042-1060] ORF |
| 167 | CCAGGGAACAUUGUCUUUG | CAAAGACAAUGUUCCCUGG | | [1039-1057] ORF |
| 168 | GUGUGCUAUUUGGGUUUGU | ACAAACCCAAAUAGCACAC | | [989-1007] ORF |
| 169 | ACCGUGUGCUAUUUGGGUU | AACCCAAAUAGCACACGGU | | [986-1004] ORF |
| 170 | GUAGGGUUAUGAGUCAGUU | AACUGACUCAUAACCCUAC | | [34-52] 5′UTR |
| 171 | ACUUGGCAGUGGCUUACUU | AAGUAAGCCACUGCCAAGU | Rat | [458-476] ORF |
| 172 | CAGAAGCAUUAACCGAGAC | GUCUCGGUUAAUGCUUCUG | | [435-453] ORF |
| 173 | GAACAUGACUGAAGCAGAG | CUCUGCUUCAGUCAUGUUC | | [405-423] ORF |
| 174 | CCUGAAGAACAUGACUGAA | UUCAGUCAUGUUCUUCAGG | | [399-417] ORF |
| 175 | GGCUCUUAAGGCUUGAAGU | ACUUCAAGCCUUAAGAGCC | | [2313-2331] 3′UTR |
| 176 | UGGCUCUUAAGGCUUGAAG | CUUCAAGCCUUAAGAGCCA | | [2312-2330] 3′UTR |
| 177 | AACCUAGCAUCUUACAAAA | UUUUGUAAGAUGCUAGGUU | | [2225-2243] 3′UTR |
| 178 | UGUAAACAUUCAUCAAGCU | AGCUUGAUGAAUGUUUACA | | [2163-2181] 3′UTR |
| 179 | GAGAGAGUCUUCCAGGUAC | GUACCUGGAAGACUCUCUC | | [2096-2114] 3′UTR |
| 180 | ACGAGAGAGUCUUCCAGGU | ACCUGGAAGACUCUCUCGU | | [2094-2112] 3′UTR |
| 181 | UGGAUGGAAGUGUCUGGAA | UUCCAGACACUUCCAUCCA | | [2070-2088] 3′UTR |
| 182 | GGAAAAGUGGAUGGAAGUG | CACUUCCAUCCACUUUUCC | | [2063-2081] 3′UTR |
| 183 | GAGGAAAAGUGGAUGGAAG | CUUCCAUCCACUUUUCCUC | | [2061-2079] 3′UTR |
| 184 | AGAGGAAAAGUGGAUGGAA | UUCCAUCCACUUUUCCUCU | | [2060-2078] 3′UTR |
| 185 | ACAUUACAGUUUGGAAGUG | CACUUCCAAACUGUAAUGU | | [1923-1941] 3′UTR |
| 186 | CAACCUGUCUUCUCUCUGU | ACAGAGAGAAGACAGGUUG | | [194-212] 5′UTR |
| 187 | GAGAAGUCUAGGAUGUUUC | GAAACAUCCUAGACUUCUC | | [1823-1841] ORF + 3′UTR |
| 188 | CUCGGAGAGAAGUCUAGGA | UCCUAGACUUCUCUCCGAG | | [1817-1835] ORF + 3′UTR |
| 189 | AAGGGAAGGUGGGCAUUUU | AAAAUGCCCACCUUCCCUU | | [1751-1769] ORF |
| 190 | GUGGAGGCACUCUUCAGUU | AACUGAAGAGUGCCUCCAC | | [1636-1654] ORF |
| 191 | CUGGAACACACUAAGCUGA | UCAGCUUAGUGUGUUCCAG | | [1411-1429] ORF |
| 192 | CCAGAAACAAAAAGAAGAG | CUCUUCUUUUUGUUUCUGG | | [1254-1272] ORF |
| 193 | CAGGCCAGAAACAAAAAGA | UCUUUUUGUUUCUGGCCUG | | [1250-1268] ORF |
| 194 | GUCUUGAAGAAGGGCAAUG | CAUUGCCCUUCUUCAAGAC | | [1057-1075] ORF |
| 195 | AGGGAACAUUGUCUUUGUC | GACAAAGACAAUGUUCCCU | | [1041-1059] ORF |
| 196 | GCCAGGGAACAUUGUCUUU | AAAGACAAUGUUCCCUGGC | | [1038-1056] ORF |
| 197 | UGCCAGGGAACAUUGUCUU | AAGACAAUGUUCCCUGGCA | | [1037-1055] ORF |
| 198 | CGUGUGCUAUUUGGGUUUG | CAAACCCAAAUAGCACACG | | [988-1006] ORF |
| 199 | GGAAAAAAGCUGAAGAACA | UGUUCUUCAGCUUUUUUCC | | [662-680] ORF |
| 200 | GCUAUCAAAGACCUUAAAG | CUUUAAGGUCUUUGAUAGC | Mouse | [520-538] ORF |
| 201 | CUCUUAAGGCUUGAAGUAA | UUACUUCAAGCCUUAAGAG | | [2315-2333] 3′UTR |
| 202 | GUAAUUGGCUCUUAAGGCU | AGCCUUAAGAGCCAAUUAC | | [2307-2325] 3′UTR |
| 203 | CUGAACCAUUACUGUAAUU | AAUUACAGUAAUGGUUCAG | | [2294-2312] 3′UTR |
| 204 | CCCAAGGAGUAAGUACAAA | UUUGUACUUACUCCUUGGG | | [2264-2282] 3′UTR |
| 205 | UCCUGUUUCUUGCUCUGAG | CUCAGAGCAAGAAACAGGA | | [2118-2136] 3′UTR |
| 206 | GGAGAGGAAAAGUGGAUGG | CCAUCCACUUUUCCUCUCC | | [2058-2076] 3′UTR |
| 207 | GGGAGAGGAAAAGUGGAUG | CAUCCACUUUUCCUCUCCC | | [2057-2075] 3′UTR |
| 208 | ACAGUUUGGAAGUGUUAAC | GUUAACACUUCCAAACUGU | | [1928-1946] 3′UTR |
| 209 | AGACAUUACAGUUUGGAAG | CUUCCAAACUGUAAUGUCU | | [1921-1939] 3′UTR |
| 210 | GAGACAUUACAGUUUGGAA | UUCCAAACUGUAAUGUCUC | | [1920-1938] 3′UTR |
| 211 | GAAAAUGAAGCCCUAUUAC | GUAAUAGGGCUUCAUUUUC | | [1860-1878] 3′UTR |
| 212 | GCUCCAACCUGUCUUCUCU | AGAGAAGACAGGUUGGAGC | | [190-208] 5′UTR |
| 213 | CCCUUUCAGAAGACAGCAU | AUGCUGUCUUCUGAAAGGG | | [1463-1481] ORF |
| 214 | GAAGUUAUCUUAAGGGAGG | CCUCCCUUAAGAUAACUUC | | [95-113] 5′UTR |
| 215 | ACAAGUACACGGUAGUCAU | AUGACUACCGUGUACUUGU | | [1325-1343] ORF |
| 216 | UGAGAAGUUAUCUUAAGGG | CCCUUAAGAUAACUUCUCA | | [92-110] 5′UTR |
| 217 | ACCAGGCCAGAAACAAAAA | UUUUUGUUUCUGGCCUGGU | | [1248-1266] ORF |
| 218 | AUGGAAAAAAGCUGAAGAA | UUCUUCAGCUUUUUUCCAU | | [660-678] ORF |
| 219 | GGAGGAAUGGAAAAAAGCU | AGCUUUUUUCCAUUCCUCC | | [654-672] ORF |
| 220 | UCCCGGAUUUGCUUCAACA | UGUUGAAGCAAAUCCGGGA | | [361-379] ORF |
| 221 | CUCCCGGAUUUGCUUCAAC | GUUGAAGCAAAUCCGGGAG | | [360-378] ORF |
| 222 | ACUCCCGGAUUUGCUUCAA | UUGAAGCAAAUCCGGGAGU | | [359-377] ORF |
| 223 | UUGGCUCUUAAGGCUUGAA | UUCAAGCCUUAAGAGCCAA | | [2311-2329] 3′UTR |
| 224 | UGAUCCUGUUUCUUGCUCU | AGAGCAAGAAACAGGAUCA | | [2115-2133] 3′UTR |
| 225 | GGUACUGAUCCUGUUUCUU | AAGAAACAGGAUCAGUACC | | [2110-2128] 3′UTR |
| 226 | GAGAGGAAAAGUGGAUGGA | UCCAUCCACUUUUCCUCUC | | [2059-2077] 3′UTR |
| 227 | CCUUCUGCUGUAUACUGUA | UACAGUAUACAGCAGAAGG | | [1899-1917] 3′UTR |
| 228 | CCAACCUGUCUUCUCUCUG | CAGAGAGAAGACAGGUUGG | | [193-211] 5′UTR |
| 229 | UCCAACCUGUCUUCUCUCU | AGAGAGAAGACAGGUUGGA | | [192-210] 5′UTR |
| 230 | AGCCCUAUUACUUGUUUGU | ACAAACAAGUAAUAGGGCU | | [1868-1888] 3′UTR |
| 231 | AUAUAAUCCUGGUGUUAUC | GAUAACACCAGGAUUAUAU | | [1697-1715] ORF |
| 232 | AGACAACAGAACCUGAGCU | AGCUGAGGUUCUGUUGUCU | | [1601-1619] ORF |
| 233 | UGAAAAAGCUGAUGCUAAU | AUUAGCAUCAGCUUUUUCA | | [1578-1596] ORF |
| 234 | CCAUGCCCUACACACUCAA | UUGAGUGUGUAGGGCAUGG | | [1298-1316] ORF |
| 235 | UGGAGUGUGUCUGGAAGCA | UGCUUCCAGACACACUCCA | | [740-758] ORF |
| 236 | UGAGUCAGUUGCCAAAAGG | CCUUUUGGCAACUGACUCA | | [43-61] 5′UTR |
| 237 | GCUGAUAGACUAUAAGAUC | GAUCUUAUAGUCUAUCAGC | | [567-585] ORF |
| 238 | CUGUUUCUGAACCAUUACU | AGUAAUGGUUCAGAAACAG | | [2288-2306] 3′UTR |
| 239 | CUGAUCCUGUUUCUUGCUC | GAGCAAGAAACAGGAUCAG | | [2114-2132] 3′UTR |
| 240 | CAGUUUGGAAGUGUUAACU | AGUUAACACUUCCAAACUG | | [1829-1947] 3′UTR |
| 241 | GGGACAUGGUGUCUAAGAA | UUCUUAGACACCAUGUCCC | | [1379-1397] ORF |
| 242 | GCCAGAAACAAAAAGAAGA | UCUUCUUUUUGUUUCUGGC | | [1253-1271] ORF |
| 243 | AGAAGGGCAAUGAUAACUG | CAGUUAUCAUUGCCCUUCU | | [1064-1082] ORF |
| 244 | CUAUUUGGGUUUGUGCCUG | CAGGCACAAACCCAAAUAG | | [994-1012] ORF |
| 245 | GAAUGGAAAAAAGCUGAAG | CUUCAGCUUUUUUCCAUUC | | [658-676] ORF |
| 246 | GGUAGGGUUAUGAGUCAGU | ACUGACUCAUAACCCUACC | | [33-51] 5′UTR |
| 247 | CUCUCAUGGUAGGGUUAUG | CAUAACCCUACCAUGAGAG | | [26-44] 5′UTR |
| 248 | UCUCUCUCAUGGUAGGGUU | AACCCUACCAUGAGAGAGA | | [23-41] 5′UTR |
| 249 | UUGGCUGCAUGUACACUAU | AUAGUGUACAUGCAGCCAA | | [380-398] ORF |
| 250 | GCCCAAGGAGUAAGUACAA | UUGUACUUACUCCUUGGGC | | [2263-2281] 3′UTR |
| 251 | CAGGUGAAUGGAGGAGUUA | UAACUCCUCCAUUCACCUG | | [2037-2055] 3′UTR |
| 252 | CAAAGCUGAAGAAAAUGAA | UUCAUUUUCUUCAGCUUUG | | [1850-1868] 3′UTR |
| 253 | CUUCUUUCCUUUUCUCUCU | AGAGAGAAAAGGAAAGAAG | | [11-29] 5′UTR |
| 254 | GAAACAAAAAGAAGAGCCU | AGGCUCUUCUUUUUGUUUC | | [1257-1275] ORF |
| 255 | CUACCUUGAACCAGUUGAG | CUCAACUGGUUCAAGGUAG | Guinea-pig | [1128-1146] ORF |
| 256 | AGGAAUGGAAAAAAGCUGA | UCAGCUUUUUUCCAUUCCU | | [656-674] ORF |
| 257 | GAGGAAUGGAAAAAAGCUG | CAGCUUUUUUCCAUUCCUC | | [655-673] ORF |
| 258 | CUCUCUCAUGGUAGGGUUA | UAACCCUACCAUGAGAGAG | | [24-42] 5′UTR |
| 259 | CCUGUUUCUGAACCAUUAC | GUAAUGGUUCAGAAACAGG | | [2287-2305] 3′UTR |
| 260 | GGUGUUAUAUAACAUUGCU | AGCAAUGUUAUAUAACACC | | [618-636] ORF |
| 261 | UGAACCAUUACUGUAAUUG | CAAUUACAGUAAUGGUUCA | | [2295-2313] 3′UTR |
| 262 | GCCUCCUAGUUUCUACCUA | UAGGUAGAAACUAGGAGGC | | [231-249] 5′UTR |
| 263 | GAAAAAGCUGAUGCUAAUA | UAUUAGCAUCAGCUUUUUC | | [1579-1597] ORF |
| 264 | GCAGUGGCUUACUUCCAAC | GUUGGAAGUAAGCCACUGC | | [463-481] ORF |
| 265 | GGCCCAAGGAGUAAGUACA | UGUACUUACUCCUUGGGCC | | [2262-2280] 3′UTR |
| 266 | UGAAGAAAAUGAAGCCCUA | UAGGGCUUCAUUUUCUUCA | | [1856-1874] 3′UTR |
| 267 | CGGUAGUCAUGAAGACUCA | UGAGUCUUCAUGACUACCG | | [1334-1352] ORF |
| 268 | GAAGGAGGAAUGGAAAAAA | UUUUUUCCAUUCCUCCUUC | | [651-669] ORF |
| 269 | GAGGUGUUAUAUAACAUUG | CAAUGUUAUAUAACACCUC | | [616-634] ORF |
| 270 | ACCAGCUGAUAGACUAUAA | UUAUAGUCUAUCAGCUGGU | | [563-581] ORF |
| 271 | CUCCUAGUUUCUACCUAAU | AUUAGGUAGAAACUAGGAG | | [233-251] 5′UTR |
| 272 | CUGUACAUUUGGUGCACUU | AAGUGCACCAAAUGUACAG | | [2180-2198] 3′UTR |
| 273 | GUGAAAAAGCUGAUGCUAA | UUAGCAUCAGCUUUUUCAC | | [1577-1595] ORF |
| 274 | UGAGAACACAGUGGGUGAC | GUCACCCACUGUGUUCUCA | | [1530-1548] ORF |
| 275 | UGAAAAACUACUGCCUGAC | GUCAGGCAGUAGUUUUUCA | Rat, Mouse | [1502-1520] ORF |
| 276 | CUCUGCUUCUUUCCUUUUC | GAAAAGGAAAGAAGCAGAG | | [6-24] 5′UTR |
| 277 | AGAAGGAGGAAUGGAAAAA | UUUUUCCAUUCCUCCUUCU | | [650-668] ORF |
| 278 | GUUUGCCUGUGAGGUGUUA | UAACACCUCACAGGCAAAC | | [606-624] ORF |
| 279 | GGCUAUCAAAGACCUUAAA | UUUAAGGUCUUUGAUAGCC | | [519-537] ORF |
| 280 | CAAACUACAAAGCUGAAGA | UCUUCAGCUUUGUAGUUUG | | [1843-1861] 3′UTR |
| 281 | GGACCUGGAGUUUCAGGAA | UUCCUGAAACUCCAGGUCC | | [1674-1692] ORF |
| 282 | GGACAUGGUGUCUAAGAAA | UUUCUUAGACACCAUGUCC | | [1380-1398] ORF |
| 283 | UCCGGGACAUGGUGUCUAA | UUAGACACCAUGUCCCGGA | | [1376-1394] ORF |
| 284 | ACACACUCAAGGUGCACUA | UAGUGCACCUUGAGUGUGU | Guinea-pig | [1307-1325] ORF |
| 285 | GCAACUACCUUGAACCAGU | ACUGGUUCAAGGUAGUUGC | Guinea-pig | [1124-1142] ORF |
| 286 | UGGAAGCAGAAGCUAUAUG | CAUAUAGCUUCUGCUUCCA | | [751-769] ORF |
| 287 | CUGGAAGCAGAAGCUAUAU | AUAUAGCUUCUGCUUCCAG | | [750-768] ORF |
| 288 | CCAAAAUCGACAAGGCGAU | AUCGCCUUGUCGAUUUUGG | | [722-740] ORF |
| 289 | AGCCCAGACAUUCCAAAAU | AUUUUGGAAUGUCUGGGCU | | [710-728] ORF |
| 290 | GCUUCAACAUUGGCUGCAU | AUGCAGCCAAUGUUGAAGC | | [371-389] ORF |
| 291 | CUAGUUUCUACCUAAUCAU | AUGAUUAGGUAGAAACUAG | | [236-254] 5′UTR + ORF |
| 292 | UCCUAGUUUCUACCUAAUC | GAUUAGGUAGAAACUAGGA | | [234-252] 5′UTR |
| 293 | CCUAGCAUCUUACAAAAAC | GUUUUUGUAAGAUGCUAGG | | [2227-2245] 3′UTR |
| 294 | ACCUAGCAUCUUACAAAAA | UUUUUGUAAGAUGCUAGGU | | [2226-2244] 3′UTR |
| 295 | CUGUGUCAUACCACAAUAA | UUAUUGUGGUAUGACACAG | | [2201-2219] 3′UTR |
| 296 | GAAGAAAAUGAAGCCCUAU | AUAGGGCUUCAUUUUCUUC | | [1857-1875] 3′UTR |
| 297 | UCACAAACUACAAAGCUGA | UCAGCUUUGUAGUUUGUGA | | [1840-1858] 3′UTR |
| 298 | GGGAUAUAAUCCUGGUGUU | AACACCAGGAUUAUAUCCC | | [1694-1712] ORF |
| 299 | UCCAGAUGAACCCAAGGAA | UUCCUUGGGUUCAUCUGGA | | [1557-1575] ORF |
| 300 | GCCAGGUGAAAAACUACUG | CAGUAGUUUUUCACCUGGC | | [1496-1514] ORF |
| 301 | CGGGACAUGGUGUCUAAGA | UCUUAGACACCAUGUCCCG | | [1378-1396] ORF |
| 302 | CUGCUUCUUUCCUUUUCUC | GAGAAAAGGAAAGAAGCAG | | [8-26] 5′UTR |
| 303 | UCUGGAAGCAGAAGCUAUA | UAUAGCUUCUGCUUCCAGA | | [749-767] ORF |
| 304 | AGGUGUUAUAUAACAUUGC | GCAAUGUUAUAUAACACCU | | [617-635] ORF |
| 305 | CUAGCAUCUUACAAAAACA | UGUUUUUGUAAGAUGCUAG | | [2228-2246] 3′UTR |
| 306 | CUUUCCUUUUCUCUCUCAU | AUGAGAGAGAAAAGGAAAG | Rat | [14-32] 5′UTR |
| 307 | GCACUUUUCUGUGUCAUAC | GUAUGACACAGAAAAGUGC | | [2193-2211] 3′UTR |
| 308 | GCUGUGUUCACACUGUAAA | UUUACAGUGUGAACACAGC | | [2150-2168] 3′UTR |
| 309 | CCUGUCUUCUCUCUGUCUC | GAGACAGAGAGAAGACAGG | | [197-215] 5′UTR |
| 310 | CUGAAGAAAAUGAAGCCCU | AGGGCUUCAUUUUCUUCAG | | [1855-1873] 3′UTR |
| 311 | ACAAACUACAAAGCUGAAG | CUUCAGCUUUGUAGUUUGU | | [1842-1860] 3′UTR |
| 312 | CACAAACUACAAAGCUGAA | UUCAGCUUUGUAGUUUGUG | | [1841-1859] 3′UTR |
| 313 | GCACUCGGAGAGAAGUCUA | UAGACUUCUCUCCGAGUGC | | [1814-1832] ORF |
| 314 | GACUGCGCAACUACAGAUU | AAUCUGUAGUUGCGCAGUC | | [1789-1807] ORF |
| 315 | AGACUGCGCAACUACAGAU | AUCUGUAGUUGCGCAGUCU | | [1788-1806] ORF |
| 316 | CCAGGUGAAAAACUACUGC | GCAGUAGUUUUUCACCUGG | | [1497-1515] ORF |
| 317 | GCAACACUGAGAAGUUAUC | GAUAACUUCUCAGUGUUGC | | [85-103] 5′UTR |
| 318 | UCUGCUUCUUUCCUUUUCU | AGAAAAGGAAAGAAGCAGA | | [7-25] 5′UTR |
| 319 | AGAAGGAUUACCUAGGCAA | UUGCCUAGGUAAUCCUUCU | | [836-854] ORF |
| 320 | CAAGAAGGAUUACCUAGGC | GCCUAGGUAAUCCUUCUUG | | [834-852] ORF |
| 321 | GAAGCUAUAUGAGCCAGUG | CACUGGCUCAUAUAGCUUC | | [759-777] ORF |
| 322 | AGAAGCUAUAUGAGCCAGU | ACUGGCUCAUAUAGCUUCU | | [758-776] ORF |
| 323 | GCAGAAGCUAUAUGAGCCA | UGGCUCAUAUAGCUUCUGC | | [756-774] ORF |
| 324 | GUGAGGUGUUAUAUAACAU | AUGUUAUAUAACACCUCAC | | [614-632] ORF |
| 325 | CCGGAUUUGCUUCAACAUU | AAUGUUGAAGCAAAUCCGG | | [363-381] ORF |
| 326 | CAGCUGUGUUCACACUGUA | UACAGUGUGAACACAGCUG | | [2148-2166] 3′UTR |
| 327 | CCCUGUUAAAAUUUAACCU | AGGUUAAAUUUUAACAGGG | | [1955-1973] 3′UTR |
| 328 | CCCUAUUACUUGUUUGUAA | UUACAAACAAGUAAUAGGG | | [1870-1888] 3′UTR |
| 329 | ACAGCAUGAAGGAUGCCUG | CAGGCAUCCUUCAUGCUGU | | [1475-1493] ORF |
| 330 | GUACACGGUAGUCAUGAAG | CUUCAUGACUACCGUGUAC | | [1329-1347] ORF |
| 331 | AGUACACGGUAGUCAUGAA | UUCAUGACUACCGUGUACU | | [1328-1346] ORF |
| 332 | AGACAGUUUCUCUGGGUUU | AAACCCAGAGAAACUGUCU | | [885-903] ORF |
| 333 | AGCUGGCCAAGAAGGAUUA | UAAUCCUUCUUGGCCAGCU | | [827-845] ORF |
| 334 | UUGCUUUCAUGUAUGCCAA | UUGGCAUACAUGAAAGCAA | | [632-650] ORF |
| 335 | CCAGCUGAUAGACUAUAAG | CUUAUAGUCUAUCAGCUGG | | [564-582] ORF |
| 336 | AACCAGCUGAUAGACUAUA | UAUAGUCUAUCAGCUGGUU | | [562-580] ORF |
| 337 | UCUGUGUCAUACCACAAUA | UAUUGUGGUAUGACACAGA | | [2200-2218] 3′UTR |
| 338 | GUGCACUUUUCUGUGUCAU | AUGACACAGAAAAGUGCAC | | [2191-2209] 3′UTR |
| 339 | GUACAUUUGGUGCACUUUU | AAAAGUGCACCAAAUGUAC | | [2182-2200] 3′UTR |
| 340 | CUCUUGGCCUCCUAGUUUC | GAAACUAGGAGGCCAAGAG | | [225-243] 5′UTR |
| 341 | UCCAGGUACUGAUCCUGUU | AACAGGAUCAGUACCUGGA | | [2106-2124] 3′UTR |
| 342 | UCUUUCCUUUUCUCUCUCA | UGAGAGAGAAAAGGAAAGA | Rat | [13-31] 5′UTR |
| 343 | GACCUGGAGUUUCAGGAAG | CUUCCUGAAACUCCAGGUC | | [1675-1693] ORF |
| 344 | AGAACCUCAGCUUAAGAAA | UUUCUUAAGCUGAGGUUCU | | [1608-1626] ORF |
| 345 | AGAACACAGUGGGUGACCA | UGGUCACCCACUGUGUUCU | | [1532-1550] ORF |
| 346 | UGGUGUCUAAGAAACUGGA | UCCAGUUUCUUAGACACCA | | [1385-1403] ORF |
| 347 | GGUUUGUGCCUGAGACAAA | UUUGUCUCAGGCACAAACC | | [1001-1019] ORF |
| 348 | CUGAUGCAUUUGCAACACU | AGUGUUGCAAAUGCAUCAG | | [74-92] 5′UTR |
| 349 | GGCUCACCGUGUGCUAUUU | AAAUAGCACACGGUGAGCC | | [981-999] ORF |
| 350 | CCUGAUGCAUUUGCAACAC | GUGUUGCAAAUGCAUCAGG | | [73-91] 5′UTR |
| 351 | GGCCAAGAAGGAUUACCUA | UAGGUAAUCCUUCUUGGCC | | [831-849] ORF |
| 352 | UGAGAGACAAGUGGCUCAG | CUGAGCCACUUGUCUCUCA | | [810-828] ORF |
| 353 | GACAUUUCCUGAUGCAUUU | AAAUGCAUCAGGAAAUGUC | | [66-84] 5′UTR |
| 354 | GGACAUUUCCUGAUGCAUU | AAUGCAUCAGGAAAUGUCC | | [65-83] 5′UTR |
| 355 | GCCCAGACAUUCCAAAAUC | GAUUUUGGAAUGUCUGGGC | | [711-729] ORF |
| 356 | GAAGUCUGAGCCCAGACAU | AUGUCUGGGCUCAGACUUC | | [702-720] ORF |
| 357 | CAGUUAGCAUUGGCCACGA | UCGUGGCCAAUGCUAACUG | | [679-697] ORF |
| 358 | GCCUGUGAGGUGUUAUAUA | UAUAUAACACCUCACAGGC | | [610-628] ORF |
| 359 | GAAAUAUGAUUUGGCUAUC | GAUAGCCAAAUCAUAUUUC | | [507-525] ORF |
| 360 | CAACGAGGGAUGCUCUACU | AGUAGAGCAUCCCUCGUUG | | [478-496] ORF |
| 361 | GAAGUAACCUUAUAGGUUA | UAACCUAUAAGGUUACUUC | | [2327-2345] 3′UTR |
| 362 | UCUGAACCAUUACUGUAAU | AUUACAGUAAUGGUUCAGA | | [2293-2311] 3′UTR |
| 363 | GUGUCAUACCACAAUAAAA | UUUUAUUGUGGUAUGACAC | | [2203-2221] 3′UTR |
| 364 | CUCUCUUGGCCUCCUAGUU | AACUAGGAGGCCAAGAGAG | | [223-241] 5′UTR |
| 365 | UGAAGCCCUAUUACUUGUU | AACAAGUAAUAGGGCUUCA | | [1865-1883] 3′UTR |
| 366 | GCACUCUUCAGUUAUGAGG | CCUCAUAACUGAAGAGUGC | | [1642-1660] ORF |
| 367 | CUGAUGCUAAUAACCAGAC | GUCUGGUUAUUAGCAUCAG | | [1586-1604] ORF |
| 368 | GCUGAUGCUAAUAACCAGA | UCUGGUUAUUAGCAUCAGC | | [1585-1603] ORF |
| 369 | CCUUUCAGAAGACAGCAUG | CAUGCUGUCUUCUGAAAGG | | [1464-1482] ORF |
| 370 | UUGCAACACUGAGAAGUUA | UAACUUCUCAGUGUUGCAA | | [83-101] 5′UTR |
| 371 | UGAGACAAAAGAAGAGCUC | GAGCUCUUCUUUUGUCUCA | | [1011-1029] ORF |
| 372 | UGAUGCAUUUGCAACACUG | CAGUGUUGCAAAUGCAUCA | | [75-93] 5′UTR |
| 373 | GUGUCUGGAAGCAGAAGCU | AGCUUCUGCUUCCAGACAC | | [746-764] ORF |
| 374 | CCAGACAUUCCAAAAUCGA | UCGAUUUUGGAAUGUCUGG | | [713-731] ORF |
| 375 | UGCCUGUGAGGUGUUAUAU | AUAUAACACCUCACAGGCA | | [609-627] ORF |
| 376 | GGCUCCAGUUCAAGCUGUU | AACAGCUUGAACUGGAGCC | | [590-608] ORF |
| 377 | AGCCUUGAUUCAGCUUCGA | UCGAAGCUGAAUCAAGGCU | | [540-558] ORF |
| 378 | AGACCUUAAAGAAGCCUUG | CAAGGCUUCUUUAAGGUCU | | [528-546] ORF |
| 379 | UGCUCUACUACCAGACAGA | UCUGUCUGGUAGUAGAGCA | | [488-506] ORF |
| 380 | CCUUUACCAGAAGCAUUAA | UUAAUGCUUCUGGUAAAGG | | [428-446] ORF |
| 381 | CUAUCCUGAAGAACAUGAC | GUCAUGUUCUUCAGGAUAG | | [395-413] ORF |
| 382 | ACCUUAUAGGUUACUCAUA | UAUGAGUAACCUAUAAGGU | | [2333-2351] 3′UTR |
| 383 | UUCUGUGUCAUACCACAAU | AUUGUGGUAUGACACAGAA | | [2199-2217] 3′UTR |
| 384 | UGCACUUUUCUGUGUCAUA | UAUGACACAGAAAAGUGCA | | [2192-2210] 3′UTR |
| 385 | GGUGCACUUUUCUGUGUCA | UGACACAGAAAAGUGCACC | | [2190-2208] 3′UTR |
| 386 | CCAGCUGUGUUCACACUGU | ACAGUGUGAACACAGCUGG | | [2147-2165] 3′UTR |
| 387 | CUAGGAUGUUUCACAAACU | AGUUUGUGAAACAUCCUAG | | [1830-1848] ORF + 3′UTR |
| 388 | CCCAGAAAGUGAACACCUU | AAGGUGUUCACUUUCUGGG | | [138-156] 5′UTR |
| 389 | UGUUAUCAAAGGUGAAUGA | UCAUUCACCUUUGAUAACA | | [1709-1727] ORF |
| 390 | GGAUAUAAUCCUGGUGUUA | UAACACCAGGAUUAUAUCC | | [1695-1713] ORF |
| 391 | GGCACUCUUCAGUUAUGAG | CUCAUAACUGAAGAGUGCC | | [1641-1659] ORF |
| 392 | AGCUGAUGCUAAUAACCAG | CUGGUUAUUAGCAUCAGCU | | [1584-1602] ORF |
| 393 | ACCAAGGCUUUCCAGAUGA | UCAUCUGGAAAGCCUUGGU | | [1547-1565] ORF |
| 394 | GACAUGGUGUCUAAGAAAC | GUUUCUUAGACACCAUGUC | | [1381-1399] ORF |
| 395 | GCACUACAAGUACACGGUA | UACCGUGUACUUGUAGUGC | | [1320-1338] ORF |
| 396 | CCUACACACUCAAGGUGCA | UGCACCUUGAGUGUGUAGG | | [1304-1322] ORF |
| 397 | GCUCCUCCUAGUUCCAAAG | CUUUGGAACUAGGAGGAGC | | [1207-1225] ORF |
| 398 | UGCAACACUGAGAAGUUAU | AUAACUUCUCAGUGUUGCA | | [84-102] 5′UTR |
| 399 | AAGACAGUUUCUCUGGGUU | AACCCAGAGAAACUGUCUU | | [884-902] ORF |
| 400 | CCAAGAAGGAUUACCUAGG | CCUAGGUAAUCCUUCUUGG | | [833-851] ORF |
| 401 | CCAAAUGAGAGACAAGUGG | CCACUUGUCUCUCAUUUGG | | [805-823] ORF |
| 402 | AAAAGCUGAAGAACAGUUA | UAACUGUUCUUCAGCUUUU | | [666-684] ORF |
| 403 | UUAUGAGUCAGUUGCCAAA | UUUGGCAACUGACUCAUAA | | [40-58] 5′UTR |
| 404 | UUGCCUGUGAGGUGUUAUA | UAUAACACCUCACAGGCAA | | [606-626] ORF |
| 405 | GCCUUGAUUCAGCUUCGAG | CUCGAAGCUGAAUCAAGGC | | [541-559] ORF |
| 406 | UGUGUCAUACCACAAUAAA | UUUAUUGUGGUAUGACACA | | [2202-2220] 3′UTR |
| 407 | AGCUGUGUUCACACUGUAA | UUACAGUGUGAACACAGCU | | [2149-2167] 3′UTR |
| 408 | CCAGGUACUGAUCCUGUUU | AAACAGGAUCAGUACCUGG | | [2107-2125] 3′UTR |
| 409 | AGACGCUCCAACCUGUCUU | AAGACAGGUUGGAGCGUCU | | [186-204] 5′UTR |
| 410 | UCAGUUAUGAGGCUACCCA | UGGGUAGCCUCAUAACUGA | | [1649-1667] ORF |
| 411 | UGACCAAGGCUUUCCAGAU | AUCUGGAAAGCCUUGGUCA | | [1545-1563] ORF |
| 412 | UCCGGCUGGAACACACUAA | UUAGUGUGUUCCAGCCGGA | | [1406-1424] ORF |
| 413 | AGCCUAAGGAAGUGAAGCU | AGCUUCACUUCCUUAGGCU | | [1271-1289] ORF |
| 414 | CCUUGAACCAGUUGAGCUG | CAGCUCAACUGGUUCAAGG | | [1131-1149] ORF |
| 415 | CAAGACAGUUUCUCUGGGU | ACCCAGAGAAACUGUCUUG | | [883-901] ORF |
| 416 | UCCUGAUGCAUUUGCAACA | UGUUGCAAAUGCAUCAGGA | | [72-90] 5′UTR |
| 417 | GGAUCAAGACAGUUUCUCU | AGAGAAACUGUCUUGAUCC | | [879-897] ORF |
| 418 | AGAGACAAGUGGCUCAGCU | AGCUGAGCCACUUGUCUCU | Guinea-pig | [812-830] ORF |
| 419 | CUGAAGAACAGUUAGCAUU | AAUGCUAACUGUUCUUCAG | | [671-689] ORF |
| 420 | AAAAAGCUGAAGAACAGUU | AACUGUUCUUCAGCUUUUU | | [665-683] ORF |
| 421 | CUUUCAUGUAUGCCAAGAA | UUCUUGGCAUACAUGAAAG | | [635-653] ORF |
| 422 | ACAUUGCUUUCAUGUAUGC | GCAUACAUGAAAGCAAUGU | | [629-647] ORF |
| 423 | UGUGAGGUGUUAUAUAACA | UGUUAUAUAACACCUCACA | | [613-631] ORF |
| 424 | CUCCAGUUCAAGCUGUUUG | CAAACAGCUUGAACUGGAG | | [592-610] ORF |
| 425 | GAACCAGCUGAUAGACUAU | AUAGUCUAUCAGCUGGUUC | | [561-579] ORF |
| 426 | GAAGCCUUGAUUCAGCUUC | GAAGCUGAAUCAAGGCUUC | | [538-556] ORF |
| 427 | AGAAGCCUUGAUUCAGCUU | AAGCUGAAUCAAGGCUUCU | | [537-555] ORF |
| 428 | GGAUGCUCUACUACCAGAC | GUCUGGUAGUAGAGCAUCC | | [485-503] ORF |
| 429 | GGGAUGCUCUACUACCAGA | UCUGGUAGUAGAGCAUCCC | | [484-502] ORF |
| 430 | GUGGCUUACUUCCAACGAG | CUCGUUGGAAGUAAGCCAC | Guinea-pig | [466-484] ORF |
| 431 | UGCUUCAACAUUGGCUGCA | UGCAGCCAAUGUUGAAGCA | | [370-388] ORF |
| 432 | CUUAUAGGUUACUCAUAAG | CUUAUGAGUAACCUAUAAG | | [2335-2353] 3′UTR |
| 433 | AGGCUUGAAGUAACCUUAU | AUAAGGUUACUUCAAGCCU | | [2321-2339] 3′UTR |
| 434 | UUCUGAACCAUUACUGUAA | UUACAGUAAUGGUUCAGAA | | [2292-2310] 3′UTR |
| 435 | GCAUCUUACAAAAACAAGA | UCUUGUUUUUGUAAGAUGC | | [2231-2249] 3′UTR |
| 436 | GCUGUACAUUUGGUGCACU | AGUGCACCAAAUGUACAGC | | [2179-2197] 3′UTR |
| 437 | UACUGAGACAUUACAGUUU | AAACUGUAAUGUCUCAGUA | | [1916-1934] 3′UTR |
| 438 | GUAAGAUUUAGCACCCUUC | GAAGGGUGCUAAAUCUUAC | | [1885-1903] 3′UTR |
| 439 | UAGGAUGUUUCACAAACUA | UAGUUUGUGAAACAUCCUA | | [1831-1849] ORF + 3′UTR |
| 440 | CAGUUAUGAGGCUACCCAA | UUGGGUAGCCUCAUAACUG | | [1650-1668] ORF |
| 441 | CUACUCAUCUGGCCCAGAA | UUCUGGGCCAGAUGAGUAG | | [126-144] 5′UTR |
| 442 | ACGGUAGUCAUGAAGACUC | GAGUCUUCAUGACUACCGU | | [1333-1351] ORF |
| 443 | ACACGGUAGUCAUGAAGAC | GUCUUCAUGACUACCGUGU | | [1331-1349] ORF |
| 444 | GGCAAUGAUAACUGGGCCA | UGGCCCAGUUAUCAUUGCC | | [1069-1087] ORF |
| 445 | UGCAUUUGCAACACUGAGA | UCUCAGUGUUGCAAAUGCA | | [78-96] 5′UTR |
| 446 | GUGCCUGAGACAAAAGAAG | CUUCUUUUGUCUCAGGCAC | | [1006-1024] ORF |
| 447 | AGCUAUAUGAGCCAGUGGU | ACCACUGGCUCAUAUAGCU | | [761-779] ORF |
| 448 | GGGACAUUUCCUGAUGCAU | AUGCAUCAGGAAAUGUCCC | | [64-82] 5′UTR |
| 449 | UGAAGUCUGAGCCCAGACA | UGUCUGGGCUCAGACUUCA | | [701-719] ORF |
| 450 | AGCUGAAGAACAGUUAGCA | UGCUAACUGUUCUUCAGCU | | [669-687] ORF |
| 451 | GCUUUCAUGUAUGCCAAGA | UCUUGGCAUACAUGAAAGC | | [634-652] ORF |
| 452 | UCAACAUUGGCUGCAUGUA | UACAUGCAGCCAAUGUUGA | | [374-392] ORF |
| 453 | CCCGGAUUUGCUUCAACAU | AUGUUGAAGCAAAUCCGGG | | [362-380] ORF |
| 454 | GUACCCAGGAUGAUUUCCU | AGGAAAUCAUCCUGGGUAC | | [1991-2009] 3′UTR |
| 455 | ACUGAGACAUUACAGUUUG | CAAACUGUAAUGUCUCAGU | | [1917-1935] 3′UTR |
| 456 | GUACUGAGACAUUACAGUU | AACUGUAAUGUCUCAGUAC | | [1915-1933] 3′UTR |
| 457 | CUGCGCAACUACAGAUUUG | CAAAUCUGUAGUUGCGCAG | | [1791-1809] ORF |
| 458 | UCCUGGUGUUAUCAAAGGU | ACCUUUGAUAACACCAGGA | | [1703-1721] ORF |
| 459 | CCUCAGCUUAAGAAAGGCA | UGCCUUUCUUAAGCUGAGG | | [1612-1630] ORF |
| 460 | GAAGCUCAGUGUUCCCAUG | CAUGGGAACACUGAGCUUC | | [1284-1302] ORF |
| 461 | GGGUUUGUGCCUGAGACAA | UUGUCUCAGGCACAAACCC | | [1000-1018] ORF |
| 462 | UGGAUCAAGACAGUUUCUC | GAGAAACUGUCUUGAUCCA | | [878-896] ORF |
| 463 | GCCAAGAAGGAUUACCUAG | CUAGGUAAUCCUUCUUGGC | | [832-850] ORF |
| 464 | UGGCCAAGAAGGAUUACCU | AGGUAAUCCUUCUUGGCCA | | [830-848] ORF |
| 465 | UGAGCCCAGACAUUCCAAA | UUUGGAAUGUCUGGGCUCA | | [708-726] ORF |
| 466 | GGCCUUUACCAGAAGCAUU | AAUGCUUCUGGUAAAGGCC | | [426-444] ORF |
| 467 | GUAACCUUAUAGGUUACUC | GAGUAACCUAUAAGGUUAC | | [2330-2348] 3′UTR |
| 468 | UGAAGUAACCUUAUAGGUU | AACCUAUAAGGUUACUUCA | | [2326-2344] 3′UTR |
| 469 | UGGUGCACUUUUCUGUGUC | GACACAGAAAAGUGCACCA | | [2189-2207] 3′UTR |
| 470 | UGUUCACACUGUAAACAUU | AAUGUUUACAGUGUGAACA | | [2154-2172] 3′UTR |
| 471 | AGGUACUGAUCCUGUUUCU | AGAAACAGGAUCAGUACCU | | [2109-2127] 3′UTR |
| 472 | UUAGCACCCUUCUGCUGUA | UACAGCAGAAGGGUGCUAA | | [1892-1910] 3′UTR |
| 473 | CCAUUCUACUCAUCUGGCC | GGCCAGAUGAGUAGAAUGG | | [121-139] 5′UTR |
| 474 | UGAAGCUCAGUGUUCCCAU | AUGGGAACACUGAGCUUCA | | [1253-1301] ORF |
| 475 | CUAAGGAAGUGAAGCUCAG | CUGAGCUUCACUUCCUUAG | | [1274-1292] ORF |
| 476 | GGCUUGUUCCCUGCAACUA | UAGUUGCAGGGAACAAGCC | Guinea-pig | [1112-1130] ORF |
| 477 | AGGCUCACCGUGUGCUAUU | AAUAGCACACGGUGAGCCU | | [980-996] ORF |
| 478 | AGACAUUCCAAAAUCGACA | UGUCGAUUUUGGAAUGUCU | | [715-733] ORF |
| 479 | CCCAGACAUUCCAAAAUCG | CGAUUUUGGAAUGUCUGGG | | [712-730] ORF |
| 480 | GCUGAAGAACAGUUAGCAU | AUGCUAACUGUUCUUCAGC | | [670-688] ORF |
| 481 | UGAUAGACUAUAAGAUCCU | AGGAUCUUAUAGUCUAUCA | Guinea-pig | [569-587] ORF |
| 482 | AAGGCUUGAAGUAACCUUA | UAAGGUUACUUCAAGCCUU | | [2320-2338] 3′UTR |
| 483 | UAAGGCUUGAAGUAACCUU | AAGGUUACUUCAAGCCUUA | | [2319-2337] 3′UTR |
| 484 | CCAUUACUGUAAUUGGCUC | GAGCCAAUUACAGUAAUGG | | [2299-2317] 3′UTR |
| 485 | AGCUGUACAUUUGGUGCAC | GUGCACCAAAUGUACAGCU | | [2178-2196] 3′UTR |
| 486 | GUGUUCACACUGUAAACAU | AUGUUUACAGUGUGAACAC | | [2153-2171] 3′UTR |
| 487 | CAGGUACUGAUCCUGUUUC | GAAACAGGAUCAGUACCUG | | [2108-2126] 3′UTR |
| 488 | GAGUCUUCCAGGUACUGAU | AUCAGUACCUGGAAGACUC | | [2100-2118] 3′UTR |
| 489 | CCUAUUACUUGUUUGUAAG | CUUACAAACAAGUAAUAGG | | [1871-1889] 3′UTR |
| 490 | GUCUAAGAAACUGGAGCUC | GAGCUCCAGUUUCUUAGAC | | [1389-1407] ORF |
| 491 | CACUACAAGUACACGGUAG | CUACCGUGUACUUGUAGUG | | [1321-1339] ORF |
| 492 | UGCACUACAAGUACACGGU | ACCGUGUACUUGUAGUGCA | | [1319-1337] ORF |
| 493 | AGGUGCACUACAAGUACAC | GUGUACUUGUAGUGCACCU | Guinea-pig | [1316-1334] ORF |
| 494 | UCAGUGUUCCCAUGCCCUA | UAGGGCAUGGGAACACUGA | | [1289-1307] ORF |
| 495 | CAACUACCUUGAACCAGUU | AACUGGUUCAAGGUAGUUG | Guinea-pig | [1125-1143] ORF |
| 496 | GUUUGUGCCUGAGACAAAA | UUUUGUCUCAGGCACAAAC | | [1002-1020] ORF |
| 497 | GAGCCCAGACAUUCCAAAA | UUUUGGAAUGUCUGGGCUC | | [709-727] ORF |
| 498 | GAAAAAAGCUGAAGAACAG | CUGUUCUUCAGCUUUUUUC | | [663-681] ORF |
| 499 | CAAGCUGUUUGCCUGUGAG | CUCACAGGCAAACAGCUUG | Mouse | [600-618] ORF |
| 500 | UCUACUACCAGACAGAGAA | UUCUCUGUCUGGUAGUAGA | | [491-509] ORF |
| 501 | GAAGCAGAGAAGGCCUUUA | UAAAGGCCUUCUCUGCUUC | | [415-433] ORF |
| 502 | AGCCAGCUGUGUUCACACU | AGUGUGAACACAGCUGGCU | | [2145-2163] 3′UTR |
| 503 | CUAGCUAGCCAGCUGUGUU | AACACAGCUGGCUAGCUAG | | [2139-2157] 3′UTR |
| 504 | UGACUCUGUGGUGUGAGAA | UUCUCACACCACAGAGUCA | | [1517-1535] ORF |
| 505 | GAGUGUGUCUGGAAGCAGA | UCUGCUUCCAGACACACUC | | [742-760] ORF |
| 506 | GGUUAUGAGUCAGUUGCCA | UGGCAACUGACUCAUAACC | | [38-56] 5′UTR |
| 507 | GCUCCAGUUCAAGCUGUUU | AAACAGCUUGAACUGGAGC | | [591-609] ORF |
| 508 | CUGAAGCAGAGAAGGCCUU | AAGGCCUUCUCUGCUUCAG | | [413-431] ORF |
| 509 | UGUAAGAUUUAGCACCCUU | AAGGGUGCUAAAUCUUACA | | [1884-1902] 3′UTR |
| 510 | CCAGAAAGUGAACACCUUG | CAAGGUGUUCACUUUCUGG | | [139-157] 5′UTR |
| 511 | GCCCAGAAAGUGAACACCU | AGGUGUUCACUUUCUGGGC | | [137-155] 5′UTR |
| 512 | UGGCCCAGAAAGUGAACAC | GUGUUCACUUUCUGGGCCA | | [135-153] 5′UTR |
| 513 | GCCAAGAAGGAGGAAUGGA | UCCAUUCCUCCUUCUUGGC | Guinea-pig | [646-664] ORF |
| 514 | AGCACCCUUCUGCUGUAUA | UAUACAGCAGAAGGGUGCU | | [1894-1912] 3′UTR |
| 515 | CAGCUGGCCAAGAAGGAUU | AAUCCUUCUUGGCCAGCUG | | [826-844] ORF |
| 516 | AGGCCUUUACCAGAAGCAU | AUGCUUCUGGUAAAGGCCU | | [425-443] ORF |
| 517 | CUAGCCAGCUGUGUUCACA | UGUGAACACAGCUGGCUAG | | [2143-2161] 3′UTR |
| 518 | UGCUCUGAGUGCUAGCUAG | CUAGCUAGCACUCAGAGCA | | [2128-2146] 3′UTR |
| 519 | UGGCUAUCAAAGACCUUAA | UUAAGGUCUUUGAUAGCCA | | [518-536] ORF |
| 520 | UAGCACCCUUCUGCUGUAU | AUACAGCAGAAGGGUGCUA | | [1893-1911] 3′UTR |
| 521 | GCUUAAGAAAGGCAGCCAA | UUGGCUGCCUUUCUUAAGC | | [1617-1635] ORF |
| 522 | CUGACUCUGUGGUGUGAGA | UCUCACACCACAGAGUCAG | | [1516-1534] ORF |
| 523 | UGAUUUGGCUAUCAAAGAC | GUCUUUGAUAGCCAAAUCA | | [513-531] ORF |
| 524 | AGAAGGCCUUUACCAGAAG | CUUCUGGUAAAGGCCUUCU | | [422-440] ORF |
| 525 | GGUCAUGCCAGGGAACAUU | AAUGUUCCCUGGCAUGACC | | [1032-1050] ORF |
| 526 | AGCUUAAGAAAGGCAGCCA | UGGCUGCCUUUCUUAAGCU | | [1516-1634] ORF |

TABLE R
21-mer oligomers for NOXA2 (NCF2, p67phox)

| | | | | |
|---|---|---|---|---|
| | | | | Human-67189969 |
| Number | Sense siRNA | AntiSense siRNA | Other Sp | ORF: 253-1833 |

| | | | | |
|---|---|---|---|---|
| 1 | GAAAAACUACUGCCUGACUCU | AGAGUCAGGCAGUAGUUUUUC | Mouse | [1503-1523] ORF |
| 2 | CCAGACAGAGAAAUAUGAUUU | AAAUCAUAUUUCUCUGUCUGG | | [496-516] ORF |
| 3 | GCAUGUACACUAUCCUGAAGA | UCUUCAGGAUAGUGUACAUGC | | [386-406] ORF |
| 4 | GAACCCAAGGAAAGUGAAAAA | UUUUUCACUUUCCUUGGGUUC | Guinea-pig | [1564-1584] ORF |
| 5 | CCCUGCAACUACCUUGAACCA | UGGUUCAAGGUAGUUGCAGGG | Guinea-pig | [1120-1140] ORF |
| 6 | CCAAGGAGUAAGUACAAAUAU | AUAUUUGUACUUACUCCUUGG | | [2265-2285] 3′UTR |
| 7 | GCGCAACUACAGAUUUGGAAA | UUUCCAAAUCUGUAGUUGCGC | | [1793-1813] ORF |
| 8 | CAAAAAGAAGAGCCUAAGGAA | UUCCUUAGGCUCUUCUUUUUG | Rat | [1261-1281] ORF |
| 9 | CGACCAAAUGAGAGACAAGUG | CACUUGUCUCUCAUUUGGUCG | | [802-822] ORF |
| 10 | CGGAGAGAAGUCUAGGAUGUU | AACAUCCUAGACUUCUCUCCG | | [1819-1839] ORF + 3′UTR |
| 11 | GGAGGCACUCUUCAGUUAUGA | UCAUAACUGAAGAGUGCCUCC | | [1638-1658] ORF |
| 12 | CCAAGGAAAGUGAAAAAGCUG | CAGCUUUUUCACUUUCCUUGG | | [1568-1588] ORF |
| 13 | GGUCAUGUUCAACGGGCAGAA | UUCUGCCCGUUGAACAUGACC | | [1089-1109] ORF |
| 14 | GAACAUUGUCUUUGUCUUGAA | UUCAAGACAAAGACAAUGUUC | | [1044-1064] ORF |
| 15 | CUGCAUGUACACUAUCCUGAA | UUCAGGAUAGUGUACAUGCAG | | [384-404] ORF |
| 16 | ACAUUCAUCAAGCUGUACAUU | AAUGUACAGCUUGAUGAAUGU | | [2168-2188] 3′UTR |
| 17 | GAAGUCUAGGAUGUUUCACAA | UUGUGAAACAUCCUAGACUUC | | [1825-1845] ORF + 3′UTR |
| 18 | AGAAGAGCCUAAGGAAGUGAA | UUCACUUCCUUAGGCUCUUCU | Rat | [1266-1286] ORF |
| 19 | CCAGGCCAGAAACAAAAAGAA | UUCUUUUUGUUUCUGGCCUGG | | [1249-1269] ORF |
| 20 | CUACUACCAGACAGAGAAAUA | UAUUUCUCUGUCUGGUAGUAG | | [492-512] ORF |
| 21 | CAUGUACACUAUCCUGAAGAA | UUCUUCAGGAUAGUGUACAUG | | [387-407] ORF |
| 22 | AGGUUACUCAUAAGGCAUAUA | UAUAUGCCUUAUGAGUAACCU | | [2340-2360] 3′UTR |
| 23 | GUAAACAUUCAUCAAGCUGUA | UACAGCUUGAUGAAUGUUUAC | | [2164-2184] 3′UTR |
| 24 | CACACUGUAAACAUUCAUCAA | UUGAUGAAUGUUUACAGUGUG | | [2158-2178] 3′UTR |
| 25 | CCCAAAGUUUUUGUUGAAGAC | GUCUUCAACAAAAACUUUGGG | | [1771-1791] ORF |
| 26 | GCUAAUAACCAGACAACAGAA | UUCUGUUGUCUGGUUAUUAGC | | [1591-1611] ORF |
| 27 | AGAUGAACCCAAGGAAAGUGA | UCACUUUCCUUGGGUUCAUCU | | [1560-1580] ORF |
| 28 | CCAGAUGAACCCAAGGAAAGU | ACUUUCCUUGGGUUCAUCUGG | | [1558-1578] ORF |
| 29 | GCAAGCUGUUUCGACCAAAUG | CAUUUGGUCGAAACAGCUUGC | | [791-811] ORF |
| 30 | GCAUUAACCGAGACAAGCACU | AGUGCUUGUCUCGGUUAAUGC | | [440-460] ORF |
| 31 | CCAGAAGCAUUAACCGAGACA | UGUCUCGGUUAAUGCUUCUGG | | [434-454] ORF |
| 32 | CGAGAGAGUCUUCCAGGUACU | AGUACCUGGAAGACUCUCUCG | | [2095-2115] 3′UTR |
| 33 | CUGUAUACUGUACUGAGACAU | AUGUCUCAGUACAGUAUACAG | | [1906-1926] 3′UTR |
| 34 | GGAUGUUUCACAAACUACAAA | UUUGUAGUUUGUGAAACAUCC | | [1833-1853] 3′UTR |
| 35 | CUCUCUCUGCUUCUUUCCUUU | AAAGGAAAGAAGCAGAGAGAG | | [2-22] 5′UTR |
| 36 | GGAGAGAAGUCUAGGAUGUUU | AAACAUCCUAGACUUCUCUCC | | [1820-1840] ORF + 3′UTR |
| 37 | CCAAAGUUUUUGUUGAAGACU | AGUCUUCAACAAAAACUUUGG | | [1772-1792] ORF |
| 38 | GUGAAUGAAGAAUGGCUGGAA | UUCCAGCCAUUCUUCAUUCAC | Rat, Mouse | [1720-1740] ORF |
| 39 | CCUGGUGUUAUCAAAGGUGAA | UUCACCUUUGAUAACACCAGG | | [1704-1724] ORF |
| 40 | GUGGAGGCACUCUUCAGUUAU | AUAACUGAAGAGUGCCUCCAC | | [1636-1656] ORF |
| 41 | UGAACCCAAGGAAAGUGAAAA | UUUUCACUUUCCUUGGGUUCA | | [1563-1583] ORF |
| 42 | GAUGAACCCAAGGAAAGUGAA | UUCACUUUCCUUGGGUUCAUC | | [1561-1581] ORF |
| 43 | GUCUUGAAGAAGGGCAAUGAU | AUCAUUGCCCUUCUUCAAGAC | | [1057-1077] ORF |
| 44 | CAUUUGCAACACUGAGAAGUU | AACUUCUCAGUGUUGCAAAUG | | [80-100] 5′UTR |
| 45 | GGGAACAUUGUCUUUGUCUUG | CAAGACAAAGACAAUGUUCCC | | [1042-1062] ORF |
| 46 | ACCUUAAAGAAGCCUUGAUUC | GAAUCAAGGCUUCUUUAAGGU | | [530-550] ORF |
| 47 | GCUAUCAAAGACCUUAAAGAA | UUCUUUAAGGUCUUUGAUAGC | | [520-540] ORF |
| 48 | CAGAGAAAUAUGAUUUGGCUA | UAGCCAAAUCAUAUUUCUCUG | | [503-523] ORF |
| 49 | CCACUCCCGGAUUUGCUUCAA | UUGAAGCAAAUCCGGGAGUGG | | [357-377] ORF |
| 50 | GGAAAAGUGGAUGGAAGUGUC | GACACUUCCAUCCACUUUUCC | | [2063-2083] 3′UTR |
| 51 | GGAGAGGAAAAGUGGAUGGAA | UUCCAUCCACUUUUCCUCUCC | | [2058-2078] 3′UTR |
| 52 | GACAAUGAUGUGAGUACCCAG | CUGGGUACUCACAUCAUUGUC | | [1978-1998] 3′UTR |
| 53 | GAAAAAGCUGAUGCUAAUAAC | GUUAUUAGCAUCAGCUUUUUC | | [1579-1599] ORF |
| 54 | CCCAAGGAAAGUGAAAAAGCU | AGCUUUUUCACUUUCCUUGGG | | [1567-1587] ORF |
| 55 | GAAGAGCCUAAGGAAGUGAAG | CUUCACUUCCUUAGGCUCUUC | Rat | [1267-1287] ORF |
| 56 | ACAAAAGAAGAGCUCCAGGUC | GACCUGGAGCUCUUCUUUUGU | | [1015-1035] ORF |
| 57 | UAGGUUACUCAUAAGGCAUAU | AUAUGCCUUAUGAGUAACCUA | | [2339-2359] 3′UTR |
| 58 | AACAUUCAUCAAGCUGUACAU | AUGUACAGCUUGAUGAAUGUU | | [2167-2187] 3′UTR |
| 59 | UCACACUGUAAACAUUCAUCA | UGAUGAAUGUUUACAGUGUGA | | [2157-2177] 3′UTR |
| 60 | CACGAGAGAGUCUUCCAGGUA | UACCUGGAAGACUCUCUCGUG | | [2093-2113] 3′UTR |
| 61 | GGACAGGUGAAUGGAGGAGUU | AACUCCUCCAUUCACCUGUCC | | [2034-2054] 3′UTR |
| 62 | CCUACUAGACAAUGAUGUGAG | CUCACAUCAUUGUCUAGUAGG | | [1971-1991] 3′UTR |
| 63 | GCAAAGGGAAGGUGGGCAUUU | AAAUGCCCACCUUCCCUUUGC | | [1748-1768] ORF |
| 64 | UGAAUGAAGAAUGGCUGGAAG | CUUCCAGCCAUUCUUCAUUCA | Rat, Mouse | [1721-1741] ORF |
| 65 | AAGGAAAGUGAAAAAGCUGAU | AUCAGCUUUUUCACUUUCCUU | | [1570-1590] ORF |
| 66 | UCAGAAGACAGCAUGAAGGAU | AUCCUUCAUGCUGUCUUCUGA | | [1468-1488] ORF |
| 67 | GUCAUGUUCAACGGGCAGAAG | CUUCUGCCCGUUGAACAUGAC | | [1090-1110] ORF |
| 68 | GCAUUUGCAACACUGAGAAGU | ACUUCUCAGUGUUGCAAAUGC | | [79-99] 5′UTR |
| 69 | GAGACAAAAGAAGAGCUCCAG | CUGGAGCUCUUCUUUUGUCUC | | [1012-1032] ORF |
| 70 | GCCUGAGACAAAAGAAGAGCU | AGCUCUUCUUUUGUCUCAGGC | | [1008-1028] ORF |
| 71 | GUGGCAUCUGUGGUGGAUCAA | UUGAUCCACCACAGAUGCCAC | Guinea-pig | [865-885] ORF |
| 72 | CCUUAAAGAAGCCUUGAUUCA | UGAAUCAAGGCUUCUUUAAGG | | [531-551] ORF |
| 73 | ACAGAGAAAUAUGAUUUGGCU | AGCCAAAUCAUAUUUCUCUGU | | [502-522] ORF |
| 74 | AGAAGCAUUAACCGAGACAAG | CUUGUCUCGGUUAAUGCUUCU | | [436-456] ORF |
| 75 | CAGAAGCAUUAACCGAGACAA | UUGUCUCGGUUAAUGCUUCUG | | [435-455] ORF |
| 76 | AGCGGACAAGAAGGACUGGAA | UUCCAGUCCUUCUUGUCCGCU | | [300-320] ORF |
| 77 | GGUUACUCAUAAGGCAUAUAC | GUAUAUGCCUUAUGAGUAACC | | [2341-2361] 3′UTR |
| 78 | UGAUGUGAGUACCCAGGAUGA | UCAUCCUGGGUACUCACAUCA | | [1983-2003] 3′UTR |
| 79 | CAAUGAUGUGAGUACCCAGGA | UCCUGGGUACUCACAUCAUUG | | [1980-2000] 3′UTR |
| 80 | ACAUUACAGUUUGGAAGUGUU | AACACUUCCAAACUGUAAUGU | | [1923-1943] 3′UTR |
| 81 | GUAUACUGUACUGAGACAUUA | UAAUGUCUCAGUACAGUAUAC | | [1908-1928] 3′UTR |
| 82 | UGUAUACUGUACUGAGACAUU | AAUGUCUCAGUACAGUAUACA | | [1907-1927] 3′UTR |
| 83 | AAGUCUAGGAUGUUUCACAAA | UUUGUGAAACAUCCUAGACUU | | [1826-1846] ORF + 3′UTR |
| 84 | AGAGAAGUCUAGGAUGUUUCA | UGAAACAUCCUAGACUUCUCU | | [1822-1842] ORF + 3′UTR |
| 85 | CAACUACAGAUUUGGAAAGCA | UGCUUUCCAAAUCUGUAGUUG | | [1796-1816] ORF |
| 86 | GCAACUACAGAUUUGGAAAGC | GCUUUCCAAAUCUGUAGUUGC | | [1795-1815] ORF |
| 87 | UGGUGUUAUCAAAGGUGAAUG | CAUUCACCUUUGAUAACACCA | | [1706-1726] ORF |
| 88 | UCACCAGGCCAGAAACAAAAA | UUUUUGUUUCUGGCCUGGUGA | | [1246-1266] ORF |
| 89 | ACAUUGUCUUUGUCUUGAAGA | UCUUCAAGACAAAGACAAUGU | | [1046-1066] ORF |
| 90 | CCAGGGAACAUUGUCUUUGUC | GACAAAGACAAUGUUCCCUGG | | [1039-1059] ORF |
| 91 | CCGUGUGCUAUUUGGGUUUGU | ACAAACCCAAAUAGCACACGG | | [967-1007] ORF |
| 92 | GGCAUCUGUGGUGGAUCAAGA | UCUUGAUCCACCACAGAUGCC | Guinea-pig | [867-887] ORF |
| 93 | AGCUGUUUCGACCAAAUGAGA | UCUCAUUUGGUCGAAACAGCU | | [794-814] ORF |
| 94 | CAGACAGAGAAAUAUGAUUUG | CAAAUCAUAUUUCUCUGUCUG | | [499-519] ORF |
| 95 | ACCAGACAGAGAAAUAUGAUU | AAUCAUAUUUCUCUGUCUGGU | | [497-517] ORF |
| 96 | CCAACGAGGGAUGCUCUACUA | UAGUAGAGCAUCCCUCGUUGG | | [477-497] ORF |
| 97 | GGCUCUUAAGGCUUGAAGUAA | UUACUUCAAGCCUUAAGAGCC | | [2313-2333] 3′UTR |
| 98 | CUGUAAACAUUCAUCAAGCUG | CAGCUUGAUGAAUGUUUACAG | | [2162-2182] 3′UTR |
| 99 | ACUGUAAACAUUCAUCAAGCU | AGCUUGAUGAAUGUUUACAGU | | [2161-2181] 3′UTR |
| 100 | UGCUGUAUACUGUACUGAGAC | GUCUCAGUACAGUAUACAGCA | | [1904-1924] 3′UTR |
| 101 | GAGAGAAGUCUAGGAUGUUUC | GAAACAUCCUAGACUUCUCUC | | [1821-1841] ORF + 3′UTR |
| 102 | CGCAACUACAGAUUUGGAAAG | CUUUCCAAAUCUGUAGUUGCG | | [1794-1814] ORF |
| 103 | UGCAAAGGGAAGGUGGGCAUU | AAUGCCCACCUUCCCUUUGCA | | [1747-1767] ORF |
| 104 | CUGGUGUUAUCAAAGGUGAAU | AUUCACCUUUGAUAACACCAG | | [1705-1725] ORF |
| 105 | CAACAGAACCUCAGCUUAAGA | UCUUAAGCUGAGGUUCUGUUG | | [1604-1624] ORF |
| 106 | AGGAAAGUGAAAAAGCUGAUG | CAUCAGCUUUUUCACUUUCCU | | [1571-1591] ORF |
| 107 | ACACUGAGAAGUUAUCUUAAG | CUUAAGAUAACUUCUCAGUGU | | [88-108] 5′UTR |
| 108 | CAAUGAUAACUGGGCCACGGU | ACCGUGGCCCAGUUAUCAUUG | | [1071-1091] ORF |
| 109 | GGAACAUUGUCUUUGUCUUGA | UCAAGACAAAGACAAUGUUCC | | [1043-1063] ORF |
| 110 | GCCAGGGAACAUUGUCUUUGU | ACAAAGACAAUGUUCCCUGGC | | [1038-1058] ORF |
| 111 | UGGUGGAUCAAGACAGUUUCU | AGAAACUGUCUUGAUCCACCA | | [875-895] ORF |
| 112 | UGGAUGGAAGUGUCUGGAAAG | CUUUCCAGACACUUCCAUCCA | | [2070-2090] 3′UTR |
| 113 | GUGGAUGGAAGUGUCUGGAAA | UUUCCAGACACUUCCAUCCAC | | [2069-2089] 3′UTR |
| 114 | GAAAAGUGGAUGGAAGUGUCU | AGACACUUCCAUCCACUUUUC | | [2064-2084] 3′UTR |
| 115 | AGAGGAAAAGUGGAUGGAAGU | ACUUCCAUCCACUUUUCCUCU | | [2050-2080] 3′UTR |
| 116 | GGGAGAGGAAAAGUGGAUGGA | UCCAUCCACUUUUCCUCUCCC | | [2057-2077] 3′UTR |
| 117 | GUGAGUACCCAGGAUGAUUUC | GAAAUCAUCCUGGGUACUCAC | | [1987-2007] 3′UTR |
| 118 | ACCCUUCUGCUGUAUACUGUA | UACAGUAUACAGCAGAAGGGU | | [1897-1917] 3′UTR |
| 119 | UGGAAAGCACUCGGAGAGAAG | CUUCUCUCCGAGUGCUUUCCA | | [1808-1828] ORF |
| 120 | UGCGCAACUACAGAUUUGGAA | UUCCAAAUCUGUAGUUGCGCA | | [1792-1812] ORF |
| 121 | CAAAGGGAAGGUGGGCAUUUU | AAAAUGCCCACCUUCCCUUUG | | [1749-1769] ORF |
| 122 | AGACAACAGAACCUCAGCUUA | UAAGCUGAGGUUCUGUUGUCU | | [1601-1621] ORF |
| 123 | UGGAACACACUAAGCUGAGCU | AGCUCAGCUUAGUGUGUUCCA | | [1412-1432] ORF |
| 124 | GAAGUUAUCUUAAGGGAGGCU | AGCCUCCCUUAAGAUAACUUC | | [95-115] 5′UTR |
| 125 | CACUGAGAAGUUAUCUUAAGG | CCUUAAGAUAACUUCUCAGUG | | [89-109] 5′UTR |
| 126 | GUGGUGGAUCAAGACAGUUUC | GAAACUGUCUUGAUCCACCAC | | [874-894] ORF |
| 127 | GCUAUAUGAGCCAGUGGUGAU | AUCACCACUGGCUCAUAUAGC | | [762-782] ORF |
| 128 | GAAUGGAAAAAAGCUGAAGAA | UUCUUCAGCUUUUUUCCAUUC | | [658-678] ORF |
| 129 | GGAAUGGAAAAAAGCUGAAGA | UCUUCAGCUUUUUUCCAUUCC | | [657-677] ORF |
| 130 | GGAGGAAUGGAAAAAAGCUGA | UCAGCUUUUUUCCAUUCCUCC | | [654-674] ORF |
| 131 | GAAGAACAUGACUGAAGCAGA | UCUGCUUCAGUCAUGUUCUUC | | [402-422] ORF |
| 132 | CUGAAGAACAUGACUGAAGCA | UGCUUCAGUCAUGUUCUUCAG | | [400-420] ORF |
| 133 | GAGUAAGUACAAAUAUUCCUG | CAGGAAUAUUUGUACUUACUC | | [2270-2290] 3′UTR |
| 134 | GCCCAAGGAGUAAGUACAAAU | AUUUGUACUUACUCCUUGGGC | | [2263-2283] 3′UTR |
| 135 | UUACAAAAACAAGACACCCAA | UUGGGUGUCUUGUUUUUGUAA | | [2236-2256] 3′UTR |
| 136 | GUACUGAUCCUGUUUCUUGCU | AGCAAGAAACAGGAUCAGUAC | | [2111-2131] 3′UTR |
| 137 | AGUGGAUGGAAGUGUCUGGAA | UUCCAGACACUUCCAUCCACU | | [2068-2088] 3′UTR |
| 138 | GACAUUACAGUUUGGAAGUGU | ACACUUCCAAACUGUAAUGUC | | [1922-1942] 3′UTR |
| 139 | GAGACAUUACAGUUUGGAAGU | ACUUCCAAACUGUAAUGUCUC | | [1920-1940] 3′UTR |
| 140 | GCUGUAUACUGUACUGAGACA | UGUCUCAGUACAGUAUACAGC | | [1905-1925] 3′UTR |
| 141 | CCUUCUGCUGUAUACUGUACU | AGUACAGUAUACAGCAGAAGG | | [1899-1919] 3′UTR |
| 142 | CAGACAACAGAACCUCAGCUU | AAGCUGAGGUUCUGUUGUCUG | | [1600-1620] ORF |
| 143 | GCUGGAACACACUAAGCUGAG | CUCAGCUUAGUGUGUUCCAGC | | [1410-1430] ORF |
| 144 | UGAGAAGUUAUCUUAAGGGAG | CUCCCUUAAGAUAACUUCUCA | | [92-112] 5′UTR |
| 145 | CUGAGAAGUUAUCUUAAGGGA | UCCCUUAAGAUAACUUCUCAG | | [91-111] 5′UTR |
| 146 | CAGGCCAGAAACAAAAAGAAG | CUUCUUUUUGUUUCUGGCCUG | | [1250-1270] ORF |
| 147 | ACCAGGCCAGAAACAAAAAGA | UCUUUUUGUUUCUGGCCUGGU | | [1248-1268] ORF |
| 148 | AGGGAACAUUGUCUUUGUCUU | AAGACAAAGACAAUGUUCCCU | | [1041-1061] ORF |
| 149 | GCUAUUUGGGUUUGUGCCUGA | UCAGGCACAAACCCAAAUAGC | | [993-1013] ORF |
| 150 | CGUGUGCUAUUUGGGUUUGUG | CACAAACCCAAAUAGCACACG | | [988-1008] ORF |
| 151 | GAACAUGACUGAAGCAGAGAA | UUCUCUGCUUCAGUCAUGUUC | | [405-425] ORF |
| 152 | AGAACAUGACUGAAGCAGAGA | UCUCUGCUUCAGUCAUGUUCU | | [404-424] ORF |
| 153 | UGGCUCUUAAGGCUUGAAGUA | UACUUCAAGCCUUAAGAGCCA | | [2312-2332] 3′UTR |
| 154 | UUACUGUAAUUGGCUCUUAAG | CUUAAGAGCCAAUUACAGUAA | | [2302-2322] 3′UTR |
| 155 | CCCAAGGAGUAAGUACAAAUA | UAUUUGUACUUACUCCUUGGG | | [2264-2284] 3′UTR |
| 156 | GAGAGAGUCUUCCAGGUACUG | CAGUACCUGGAAGACUCUCUC | | [2096-2116] 3′UTR |
| 157 | UGGAAGUGUCUGGAAAGGGCA | UGCCCUUUCCAGACACUUCCA | | [2074-2094] 3′UTR |
| 158 | CAUUACAGUUUGGAAGUGUUA | UAACACUUCCAAACUGUAAUG | | [1924-1944] 3′UTR |
| 159 | AGAUUUGGAAAGCACUCGGAG | CUCCGAGUGCUUUCCAAAUCU | | [1803-1823] ORF |
| 160 | CAGAUUUGGAAAGCACUCGGA | UCCGAGUGCUUUCCAAAUCUG | | [1802-1822] ORF |
| 161 | GCUUCUUUCCUUUUCUCUCUC | GAGAGAGAAAAGGAAAGAAGC | | [10-30] 5′UTR |
| 162 | AAGGAAGUGAAGCUCAGUGUU | AACACUGAGCUUCACUUCCUU | | [1276-1296] ORF |
| 163 | CUUUGUCUUGAAGAAGGGCAA | UUGCCCUUCUUCAAGACAAAG | | [1053-1073] ORF |
| 164 | CAGGGAACAUUGUCUUUGUCU | AGACAAAGACAAUGUUCCCUG | | [1040-1060] ORF |
| 165 | CUGGAAGCAGAAGCUAUAUGA | UCAUAUAGCUUCUGCUUCCAG | | [750-770] ORF |
| 166 | UGAAGAACAGUUAGCAUUGGC | GCCAAUGCUAACUGUUCUUCA | | [672-692] ORF |
| 167 | GAGGAAUGGAAAAAAGCUGAA | UUCAGCUUUUUUCCAUUCCUC | | [655-675] ORF |
| 168 | CCUGAAGAACAUGACUGAAGC | GCUUCAGUCAUGUUCUUCAGG | | [399-419] ORF |
| 169 | UGUAAUUGGCUCUUAAGGCUU | AAGCCUUAAGAGCCAAUUACA | | [2306-2326] 3′UTR |
| 170 | CAUUACUGUAAUUGGCUCUUA | UAAGAGCCAAUUACAGUAAUG | | [2300-2320] 3′UTR |
| 171 | UGAUCCUGUUUCUUGCUCUGA | UCAGAGCAAGAAACAGGAUCA | | [2115-2135] 3′UTR |
| 172 | GACAGGUGAAUGGAGGAGUUA | UAACUCCUCCAUUCACCUGUC | | [2035-2055] 3′UTR |
| 173 | CAGUUUGGAAGUGUUAACUAU | AUAGUUAACACUUCCAAACUG | | [1929-1949] 3′UTR |
| 174 | CCCUUCUGCUGUAUACUGUAC | GUACAGUAUACAGCAGAAGGG | | [1896-1918] 3′UTR |
| 175 | AGAAAAUGAAGCCCUAUUACU | AGUAAUAGGGCUUCAUUUUCU | | [1859-1879] 3′UTR |
| 176 | GAAGAAAAUGAAGCCCUAUUA | UAAUAGGGCUUCAUUUUCUUC | | [1857-1877] 3′UTR |
| 177 | GAUAUAAUCCUGGUGUUAUCA | UGAUAACACCAGGAUUAUAUC | | [1696-1716] ORF |
| 178 | GACAACAGAACCUCAGCUUAA | UUAAGCUGAGGUUCUGUUGUC | | [1602-1622] ORF |
| 179 | CCAGACAACAGAACCUCAGCU | AGCUGAGGUUCUGUUGUCUGG | | [1599-1619] ORF |
| 180 | GGCCAGAAACAAAAAGAAGAG | CUCUUCUUUUUGUUUCUGGCC | | [1252-1272] ORF |
| 181 | GAAGAAGGGCAAUGAUAACUG | CAGUUAUCAUUGCCCUUCUUC | | [1062-1082] ORF |
| 182 | CUAUUUGGGUUUGUGCCUGAG | CUCAGGCACAAACCCAAAUAG | | [994-1014] ORF |
| 183 | UGGUAGGGUUAUGAGUCAGUU | AACUGACUCAUAACCCUACCA | | [32-52] 5′UTR |
| 184 | UCCUUUUCUCUCUCAUGGUAG | CUACCAUGAGAGAGAAAAGGA | | [17-37] 5′UTR |
| 185 | CACUCCCGGAUUUGCUUCAAC | GUUGAAGCAAAUCCGGGAGUG | | [358-378] ORF |
| 186 | GCUCUUAAGGCUUGAAGUAAC | GUUACUUCAAGCCUUAAGAGC | | [2314-2334] 3′UTR |
| 187 | UCCUGUUUCUUGCUCUGAGUG | CACUCAGAGCAAGAAACAGGA | | [2118-2138] 3′UTR |
| 188 | CUGAUCCUGUUUCUUGCUCUG | CAGAGCAAGAAACAGGAUCAG | | [2114-2134] 3′UTR |
| 189 | CUCCAACCUGUCUUCUCUCUG | CAGAGAGAAGACAGGUUGGAG | | [191-211] 5′UTR |
| 190 | AGAAACAAAAAGAAGAGCCUA | UAGGCUCUUCUUUUUGUUUCU | | [1256-1276] ORF |
| 191 | ACCGUGUGCUAUUUGGGUUUG | CAAACCCAAAUAGCACACGGU | | [986-1006] ORF |
| 192 | GGUAGGGUUAUGAGUCAGUUG | CAACUGACUCAUAACCCUACC | | [33-53] 5′UTR |
| 193 | AUCCUGUUUCUUGCUCUGAGU | ACUCAGAGCAAGAAACAGGAU | | [2117-2137] 3′UTR |
| 194 | GUGUUAACUAUUUAUUCCCUG | CAGGGAAUAAAUAGUUAACAC | | [1939-1959] 3′UTR |
| 195 | GUGAAAAAGCUGAUGCUAAUA | UAUUAGCAUCAGCUUUUUCAC | | [1577-1597] ORF |
| 196 | CGGGACAUGGUGUCUAAGAAA | UUUCUUAGACACCAUGUCCCG | | [1378-1398] ORF |
| 197 | GUAUGCCAAGAAGGAGGAAUG | CAUUCCUCCUUCUUGGCAUAC | | [642-662] ORF |
| 198 | CAUUGGCUGCAUGUACACUAU | AUAGUGUACAUGCAGCCAAUG | | [378-398] ORF |
| 199 | GAGAACACAGUGGGUGACCAA | UUGGUCACCCACUGUGUUCUC | | [1531-1551] ORF |
| 200 | GAAACAAAAAGAAGAGCCUAA | UUAGGCUCUUCUUUUUGUUUC | | [1257-1277] ORF |
| 201 | CCAGAAACAAAAAGAAGAGCC | GGCUCUUCUUUUUGUUUCUGG | | [1254-1274] ORF |
| 202 | AGGAAUGGAAAAAAGCUGAAG | CUUCAGCUUUUUUCCAUUCCU | | [656-676] ORF |
| 203 | UUCUCUCUCAUGGUAGGGUUA | UAACCCUACCAUGAGAGAGAA | | [22-42] 5′UTR |
| 204 | GGCCCAAGGAGUAAGUACAAA | UUUGUACUUACUCCUUGGGCC | | [2262-2282] 3′UTR |
| 205 | UCCUAGUUUCUACCUAAUCAU | AUGAUUAGGUAGAAACUAGGA | | [234-254] 5′UTR + ORF |
| 206 | GCCUCCUAGUUUCUACCUAAU | AUUAGGUAGAAACUAGGAGGC | | [231-251] 5′UTR |
| 207 | GCUGAAGAAAAUGAAGCCCUA | UAGGGCUUCAUUUUCUUCAGC | | [1854-1874] 3′UTR |
| 208 | CAGAAACAAAAAGAAGAGCCU | AGGCUCUUCUUUUUGUUUCUG | | [1255-1275] ORF |
| 209 | AGGCCCAAGGAGUAAGUACAA | UUGUACUUACUCCUUGGGCCU | | [2261-2281] 3′UTR |
| 210 | CCUCCUAGUUUCUACCUAAUC | GAUUAGGUAGAAACUAGGAGG | | [232-252] 5′UTR |
| 211 | GGCCUCCUAGUUUCUACCUAA | UUAGGUAGAAACUAGGAGGCC | | [230-250] 5′UTR |
| 212 | CUGAAGAAAAUGAAGCCCUAU | AUAGGGCUUCAUUUUCUUCAG | | [1855-1875] 3′UTR |
| 213 | CAAACUACAAAGCUGAAGAAA | UUUCUUCAGCUUUGUAGUUUG | | [1843-1863] 3′UTR |
| 214 | ACAAACUACAAAGCUGAAGAA | UUCUUCAGCUUUGUAGUUUGU | | [1842-1862] 3′UTR |
| 215 | CCCUUUCAGAAGACAGCAUGA | UCAUGCUGUCUUCUGAAAGGG | | [1463-1483] ORF |
| 216 | GCCAGAAACAAAAAGAAGAGC | GCUCUUCUUUUUGUUUCUGGC | | [1253-1273] ORF |
| 217 | UCUCUCUGCUUCUUUCCUUUU | AAAAGGAAAGAAGCAGAGAGA | | [3-23] 5′UTR |
| 218 | CGGAUUUGCUUCAACAUUGGC | GCCAAUGUUGAAGCAAAUCCG | | [384-384] ORF |
| 219 | CCAACCUGUCUUCUCUCUGUC | GACAGAGAGAAGACAGGUUGG | | [193-213] 5′UTR |
| 220 | ACAAAGCUGAAGAAAAUGAAG | CUUCAUUUUCUUCAGCUUUGU | | [1849-1869] 3′UTR |
| 221 | UCCCAUGCCCUACACACUCAA | UUGAGUGUGUAGGGCAUGGGA | | [1296-1316] ORF |
| 222 | CCAAGAAGGAGGAAUGGAAAA | UUUUCCAUUCCUCCUUCUUGG | Guinea-pig | [647-667] ORF |
| 223 | GAACCAGCUGAUAGACUAUAA | UUAUAGUCUAUCAGCUGGUUC | | [561-581] ORF |
| 224 | GGAUUUGCUUCAACAUUGGCU | AGCCAAUGUUGAAGCAAAUCC | | [365-385] ORF |
| 225 | CCUAGCAUCUUACAAAAACAA | UUGUUUUUGUAAGAUGCUAGG | | [2227-2247] 3′UTR |
| 226 | GGUGCACUUUUCUGUGUCAUA | UAUGACACAGAAAAGUGCACC | | [2190-2210] 3′UTR |
| 227 | GCUGUACAUUUGGUGCACUUU | AAAGUGCACCAAAUGUACAGC | | [2179-2199] 3′UTR |
| 228 | CAGCUGUGUUCACACUGUAAA | UUUACAGUGUGAACACAGCUG | | [2148-2168] 3′UTR |
| 229 | UGAAGAAAAUGAAGCCCUAUU | AAUAGGGCUUCAUUUUCUUCA | | [1856-1876] 3′UTR |
| 230 | GGUGAAAAACUACUGCCUGAC | GUCAGGCAGUAGUUUUUCACC | | [1500-1520] ORF |
| 231 | CCAGGUGAAAAACUACUGCCU | AGGCAGUAGUUUUUCACCUGG | | [1497-1517] ORF |
| 232 | CCGGGACAUGGUGUCUAAGAA | UUCUUAGACACCAUGUCCCGG | | [1377-1397] ORF |
| 233 | ACAUCCCAGCUCCUCCUAGUU | AACUAGGAGGAGCUGGGAUGU | | [1199-1219] ORF |
| 234 | GCAGAAGCUAUAUGAGCCAGU | ACUGGCUCAUAUAGCUUCUGC | | [756-776] ORF |
| 235 | GCAGUGGCUUACUUCCAACGA | UCGUUGGAAGUAAGCCACUGC | | [463-483] ORF |
| 236 | ACCUAGCAUCUUACAAAAACA | UGUUUUUGUAAGAUGCUAGGU | | [2226-2246] 3′UTR |
| 237 | UUCUGUGUCAUACCACAAUAA | UUAUUGUGGUAUGACACAGAA | | [2199-2219] 3′UTR |
| 238 | GCCAGCUGUGUUCACACUGUA | UACAGUGUGAACACAGCUGGC | | [2146-2168] 3′UTR |
| 239 | CACUCUUCAGUUAUGAGGCUA | UAGCCUCAUAACUGAAGAGUG | | [1643-1663] ORF |
| 240 | GUGAAAAACUACUGCCUGACU | AGUCAGGCAGUAGUUUUUCAC | Mouse | [1501-1521] ORF |
| 241 | CUCUGCUUCUUUCCUUUUCUC | GAGAAAAGGAAAGAAGCAGAG | | [6-26] 5′UTR |
| 242 | UCUCUGCUUCUUUCCUUUUCU | AGAAAAGGAAAGAAGCAGAGA | | [5-25] 5′UTR |
| 243 | GAGGUGUUAUAUAACAUUGCU | AGCAAUGUUAUAUAACACCUC | | [616-636] ORF |
| 244 | GGGUUAUGAGUCAGUUGCCAA | UUGGCAACUGACUCAUAACCC | | [37-57] 5′UTR |
| 245 | GACCUUAAAGAAGCCUUGAUU | AAUCAAGGCUUCUUUAAGGUC | | [529-549] ORF |
| 246 | CACUAUCCUGAAGAACAUGAC | GUCAUGUUCUUCAGGAUAGUG | | [393-413] ORF |
| 247 | CAACAUUGGCUGCAUGUACAC | GUGUACAUGCAGCCAAUGUUG | | [375-395] ORF |
| 248 | AGCUGUACAUUUGGUGCACUU | AAGUGCACCAAAUGUACAGCU | | [2178-2198] 3′UTR |
| 249 | ACAGAACCUCAGCUUAAGAAA | UUUCUUAAGCUGAGGUUCUGU | | [1606-1626] ORF |
| 250 | AGUGAAAAAGCUGAUGCUAAU | AUUAGCAUCAGCUUUUUCACU | | [1576-1596] ORF |
| 251 | GGCUUUCCAGAUGAACCCAAG | CUUGGGUUCAUCUGGAAAGCC | | [1552-1572] ORF |
| 252 | GGACAUGGUGUCUAAGAAACU | AGUUUCUUAGACACCAUGUCC | | [1380-1400] ORF |
| 253 | UCAAGACAGUUUCUCUGGGUU | AACCCAGAGAAACUGUCUUGA | | [882-902] ORF |
| 254 | CCUGAUGCAUUUGCAACACUG | CAGUGUUGCAAAUGCAUCAGG | | [73-93] 5′UTR |
| 255 | GGCAAGCUGUUUCGACCAAAU | AUUUGGUCGAAACAGCUUGCC | | [790-810] ORF |
| 256 | GGGCAAGCUGUUUCGACCAAA | UUUGGUCGAAACAGCUUGCCC | | [789-809] ORF |
| 257 | AGAAGGAGGAAUGGAAAAAAG | CUUUUUUCCAUUCCUCCUUCU | | [650-670] ORF |
| 258 | CAAGAAGGAGGAAUGGAAAAA | UUUUUCCAUUCCUCCUUCUUG | Guinea-pig | [648-668] ORF |
| 259 | GUGAGGUGUUAUAUAACAUUG | CAAUGUUAUAUAACACCUCAC | | [614-634] ORF |
| 260 | GGGAACCAGCUGAUAGACUAU | AUAGUCUAUCAGCUGGUUCCC | | [559-579] ORF |
| 261 | GCCAUCAGCCUCUGGAAUGAA | UUCAUUCCAGAGGCUGAUGGC | | [268-288] ORF |
| 262 | CUCCUAGUUUCUACCUAAUCA | UGAUUAGGUAGAAACUAGGAG | | [233-253] 5′UTR |
| 263 | UUCUUUCCUUUUCUCUCUCAU | AUGAGAGAGAAAAGGAAAGAA | | [12-32] 5′UTR |
| 264 | CAACCUGUCUUCUCUCUGUCU | AGACAGAGAGAAGACAGGUUG | | [194-214] 5′UTR |
| 265 | CACAAACUACAAAGCUGAAGA | UCUUCAGCUUUGUAGUUUGUG | | [1841-1861] 3′UTR |
| 266 | AGACUGCGCAACUACAGAUUU | AAAUCUGUAGUUGCGCAGUCU | | [1788-1808] ORF |
| 267 | GUGUUAUCAAAGGUGAAUGAA | UUCAUUCACCUUUGAUAACAC | | [1708-1728] ORF |
| 268 | CUGAUGCUAAUAACCAGACAA | UUGUCUGGUUAUUAGCAUCAG | | [1586-1606] ORF |
| 269 | GACCAAGGCUUUCCAGAUGAA | UUCAUCUGGAAAGCCUUGGUC | | [1546-1568] ORF |
| 270 | CUACACACUCAAGGUGCACUA | UAGUGCACCUUGAGUGUGUAG | | [1305-1325] ORF |
| 271 | UCCUGAUGCAUUUGCAACACU | AGUGUUGCAAAUGCAUCAGGA | | [72-92] 5′UTR |
| 272 | CAAGAAGGAUUACCUAGGCAA | UUGCCUAGGUAAUCCUUCUUG | | [834-854] ORF |
| 273 | CCAAGAAGGAUUACCUAGGCA | UGCCUAGGUAAUCCUUCUUGG | | [833-853] ORF |
| 274 | GGGCUCCAGUUCAAGCUGUUU | AAACAGCUUGAACUGGAGCCC | | [589-609] ORF |
| 275 | GGAACCAGCUGAUAGACUAUA | UAUAGUCUAUCAGCUGGUUCC | | [560-580] ORF |
| 276 | GUACACUAUCCUGAAGAACAU | AUGUUCUUCAGGAUAGUGUAC | | [390-410] ORF |
| 277 | GUACAUUUGGUGCACUUUUCU | AGAAAAGUGCACCAAAUGUAC | | [2182-2202] 3′UTR |
| 278 | CUGUACAUUUGGUGCACUUUU | AAAAGUGCACCAAAUGUACAG | | [2180-2200] 3′UTR |
| 279 | GUACUGAGACAUUACAGUUUG | CAAACUGUAAUGUCUCAGUAC | | [1915-1935] 3′UTR |
| 280 | GAGGACCUGGAGUUUCAGGAA | UUCCUGAAACUCCAGGUCCUC | | [1672-1692] ORF |
| 281 | CAGGUGAAAAACUACUGCCUG | CAGGCAGUAGUUUUUCACCUG | | [1498-1518] ORF |
| 282 | GGGCCAGGUGAAAAACUACUG | CAGUAGUUUUUCACCUGGCCC | | [1494-1514] ORF |
| 283 | AGACAGCAUGAAGGAUGCCUG | CAGGCAUCCUUCAUGCUGUCU | | [1473-1493] ORF |
| 284 | GGAACACACUAAGCUGAGCUA | UAGCUCAGCUUAGUGUGUUCC | | [1413-1433] ORF |
| 285 | GGGACAUGGUGUCUAAGAAAC | GUUUCUUAGACACCAUGUCCC | | [1379-1399] ORF |
| 286 | CACGGUAGUCAUGAAGACUCA | UGAGUCUUCAUGACUACCGUG | | [1332-1352] ORF |
| 287 | CAAGUACACGGUAGUCAUGAA | UUCAUGACUACCGUGUACUUG | | [1326-1346] ORF |
| 288 | GGUUUGUGCCUGAGACAAAAG | CUUUUGUCUCAGGCACAAACC | | [1001-1021] ORF |
| 289 | CUGGCCAAGAAGGAUUACCUA | UAGGUAAUCCUUCUUGGCCAG | | [829-849] ORF |
| 290 | UGGAAGCAGAAGCUAUAUGAG | CUCAUAUAGCUUCUGCUUCCA | | [751-771] ORF |
| 291 | GGAGUGUGUCUGGAAGCAGAA | UUCUGCUUCCAGACACACUCC | | [741-761] ORF |
| 292 | CAAAAUCGACAAGGCGAUGGA | UCCAUCGCCUUGUCGAUUUUG | | [723-743] ORF |
| 293 | CAGACAUUCCAAAAUCGACAA | UUGUCGAUUUUGGAAUGUCUG | | [714-734] ORF |
| 294 | AAGAAGGAGGAAUGGAAAAAA | UUUUUUCCAUUCCUCCUUCUU | | [649-669] ORF |
| 295 | UGCUUUCAUGUAUGCCAAGAA | UUCUUGGCAUACAUGAAAGCA | | [633-653] ORF |
| 296 | CAUUGCUUUCAUGUAUGCCAA | UUGGCAUACAUGAAAGCAAUG | | [630-650] ORF |
| 297 | UGAGGUGUUAUAUAACAUUGC | GCAAUGUUAUAUAACACCUCA | | [615-635] ORF |
| 298 | CUGUGAGGUGUUAUAUAACAU | AUGUUAUAUAACACCUCACAG | | [612-632] ORF |
| 299 | UGCCUGUGAGGUGUUAUAUAA | UUAUAUAACACCUCACAGGCA | | [609-629] ORF |
| 300 | GGCUGCAUGUACACUAUCCUG | CAGGAUAGUGUACAUGCAGCC | | [382-402] ORF |
| 301 | UGGCUGCAUGUACACUAUCCU | AGGAUAGUGUACAUGCAGCCA | | [381-401] ORF |
| 302 | GUACAAAUAUUCCUGUUUCUG | CAGAAACAGGAAUAUUUGUAC | | [2276-2296] 3′UTR |
| 303 | UCUGUGUCAUACCACAAUAAA | UUUAUUGUGGUAUGACACAGA | | [2200-2220] 3′UTR |
| 304 | CCAGCUGUGUUCACACUGUAA | UUACAGUGUGAACACAGCUGG | | [2147-2167] 3′UTR |
| 305 | GCUCUCUUGGCCUCCUAGUUU | AAACUAGGAGGCCAAGAGAGC | | [222-242] 5′UTR |
| 306 | CUACUAGACAAUGAUGUGAGU | ACUCACAUCAUUGUCUAGUAG | | [1972-1992] 3′UTR |
| 307 | CCCUAUUACUUGUUUGUAAGA | UCUUACAAACAAGUAAUAGGG | | [1870-1890] 3′UTR |
| 308 | CUUCUUUCCUUUUCUCUCUCA | UGAGAGAGAAAAGGAAAGAAG | | [11-31] 5′UTR |
| 309 | GGUGUUAUCAAAGGUGAAUGA | UCAUUCACCUUUGAUAACACC | | [1707-1727] ORF |
| 310 | GCUGAUGCUAAUAACCAGACA | UGUCUGGUUAUUAGCAUCAGC | | [1585-1605] ORF |
| 311 | CAACACUGAGAAGUUAUCUUA | UAAGAUAACUUCUCAGUGUUG | | [86-106] 5′UTR |
| 312 | CCAGCUCCUCCUAGUUCCAAA | UUUGGAACUAGGAGGAGCUGG | | [1204-1224] ORF |
| 313 | UAACUGGGCCACGGUCAUGUU | AACAUGACCGUGGCCCAGUUA | | [1077-1097] ORF |
| 314 | GGAUCAAGACAGUUUCUCUGG | CCAGAGAAACUGUCUUGAUCC | | [879-899] ORF |
| 315 | GCAUCUGUGGUGGAUCAAGAC | GUCUUGAUCCACCACAGAUGC | Guinea-pig | [868-888] ORF |
| 316 | GUCUGGAAGCAGAAGCUAUAU | AUAUAGCUUCUGCUUCCAGAC | | [748-768] ORF |
| 317 | GGACAUUUCCUGAUGCAUUUG | CAAAUGCAUCAGGAAAUGUCC | | [65-85] 5′UTR |
| 318 | ACAGUUAGCAUUGGCCACGAG | CUCGUGGCCAAUGCUAACUGU | | [678-698] ORF |
| 319 | GCCUGUGAGGUGUUAUAUAAC | GUUAUAUAACACCUCACAGGC | | [610-630] ORF |
| 320 | AGACCUUAAAGAAGCCUUGAU | AUCAAGGCUUCUUUAAGGUCU | | [528-548] ORF |
| 321 | GCUUACUUCCAACGAGGGAUG | CAUCCCUCGUUGGAAGUAAGC | Guinea-pig | [469-489] ORF |
| 322 | GCACUUGGCAGUGGCUUACUU | AAGUAAGCCACUGCCAAGUGC | Rat | [456-476] ORF |
| 323 | GGCCUUUACCAGAAGCAUUAA | UUAAUGCUUCUGGUAAAGGCC | | [426-446] ORF |
| 324 | GCUUCAACAUUGGCUGCAUGU | ACAUGCAGCCAAUGUUGAAGC | | [371-391] ORF |
| 325 | CCGGAUUUGCUUCAACAUUGG | CCAAUGUUGAAGCAAAUCCGG | | [363-383] ORF |
| 326 | ACCUUAUAGGUUACUCAUAAG | CUUAUGAGUAACCUAUAAGGU | | [2333-2353] 3′UTR |
| 327 | CUGUGUCAUACCACAAUAAAA | UUUUAUUGUGGUAUGACACAG | | [2201-2221] 3′UTR |
| 328 | CCCUGUUAAAAUUUAACCUAC | GUAGGUUAAAUUUUAACAGGG | | [1955-1975] 3′UTR |
| 329 | UGAUGCUAAUAACCAGACAAC | GUUGUCUGGUUAUUAGCAUCA | | [1587-1607] ORF |
| 330 | UGACCAAGGCUUUCCAGAUGA | UCAUCUGGAAAGCCUUGGUCA | | [1545-1565] ORF |
| 331 | ACAGUGGGUGACCAAGGCUUU | AAAGCCUUGGUCACCCACUGU | | [1537-1557] ORF |
| 332 | GUGUGAGAACACAGUGGGUGA | UCACCCACUGUGUUCUCACAC | Guinea-pig | [1527-1547] ORF |
| 333 | GGUGUCUAAGAAACUGGAGCU | AGCUCCAGUUUCUUAGACACC | | [1386-1406] ORF |
| 334 | ACGGUAGUCAUGAAGACUCAG | CUGAGUCUUCAUGACUACCGU | | [1333-1353] ORF |
| 335 | GCAACACUGAGAAGUUAUCUU | AAGAUAACUUCUCAGUGUUGC | | [85-105] 5′UTR |
| 336 | GUUCCCUGCAACUACCUUGAA | UUCAAGGUAGUUGCAGGGAAC | Guinea-pig | [1117-1137] ORF |
| 337 | CAAGACAGUUUCUCUGGGUUU | AAACCCAGAGAAACUGUCUUG | | [883-903] ORF |
| 338 | GGGACAUUUCCUGAUGCAUUU | AAAUGCAUCAGGAAAUGUCCC | | [64-84] 5′UTR |
| 339 | UGAAGUCUGAGCCCAGACAUU | AAUGUCUGGGCUCAGACUUCA | | [701-721] ORF |
| 340 | AUGAAGUCUGAGCCCAGACAU | AUGUCUGGGCUCAGACUUCAU | | [700-720] ORF |
| 341 | ACCAGCUGAUAGACUAUAAGA | UCUUAUAGUCUAUCAGCUGGU | | [563-583] ORF |
| 342 | CAACGAGGGAUGCUCUACUAC | GUAGUAGAGCAUCCCUCGUUG | | [478-498] ORF |
| 343 | UUCCUGUUUCUGAACCAUUAC | GUAAUGGUUCAGAAACAGGAA | | [2285-2305] 3′UTR |
| 344 | CUUUUCUGUGUCAUACCACAA | UUGUGGUAUGACACAGAAAAG | | [2196-2216] 3′UTR |
| 345 | UACUAGACAAUGAUGUGAGUA | UACUCACAUCAUUGUCUAGUA | | [1973-1993] 3′UTR |
| 346 | AGGAUGUUUCACAAACUACAA | UUGUAGUUUGUGAAACAUCCU | | [1832-1852] ORF + 3′UTR |
| 347 | GAAGACUGCGCAACUACAGAU | AUCUGUAGUUGCGCAGUCUUC | | [1786-1806] ORF |
| 348 | GGCCAGGUGAAAAACUACUGC | GCAGUAGUUUUUCACCUGGCC | | [1495-1515] ORF |
| 349 | CUACAAGUACACGGUAGUCAU | AUGACUACCGUGUACUUGUAG | | [1323-1343] ORF |
| 350 | GCACUACAAGUACACGGUAGU | ACUACCGUGUACUUGUAGUGC | | [1320-1340] ORF |
| 351 | ACACUCAAGGUGCACUACAAG | CUUGUAGUGCACCUUGAGUGU | Guinea-pig | [1309-1329] ORF |
| 352 | CACACUCAAGGUGCACUACAA | UUGUAGUGCACCUUGAGUGUG | Guinea-pig | [1308-1328] ORF |
| 353 | UGGGUUUGUGCCUGAGACAAA | UUUGUCUCAGGCACAAACCCA | | [999-1019] ORF |
| 354 | CCAGACAUUCCAAAAUCGACA | UGUCGAUUUUGGAAUGUCUGG | | [713-733] ORF |
| 355 | GUUAUGAGUCAGUUGCCAAAA | UUUUGGCAACUGACUCAUAAC | | [39-59] 5′UTR |
| 356 | CCAGCUGAUAGACUAUAAGAU | AUCUUAUAGUCUAUCAGCUGG | | [564-584] ORF |
| 357 | AAAGAAGCCUUGAUUCAGCUU | AAGCUGAAUCAAGGCUUCUUU | | [535-555] ORF |
| 358 | CAAAGACCUUAAAGAAGCCUU | AAGGCUUCUUUAAGGUCUUUG | | [525-545] ORF |
| 359 | UGCUUCAACAUUGGCUGCAUG | CAUGCAGCCAAUGUUGAAGCA | | [370-390] ORF |
| 360 | CCCGGAUUUGCUUCAACAUUG | CAAUGUUGAAGCAAAUCCGGG | | [362-382] ORF |
| 361 | GUAACCUUAUAGGUUACUCAU | AUGAGUAACCUAUAAGGUUAC | | [2330-2350] 3′UTR |
| 362 | CCUAGUUUCUACCUAAUCAUG | CAUGAUUAGGUAGAAACUAGG | | [235-255] 5′UTR + ORF |
| 363 | GCACUUUUCUGUGUCAUACCA | UGGUAUGACACAGAAAAGUGC | | [2193-2213] 3′UTR |
| 364 | UGGUGCACUUUUCUGUGUCAU | AUGACACAGAAAAGUGCACCA | | [2189-2209] 3′UTR |
| 365 | CCAGGUACUGAUCCUGUUUCU | AGAAACAGGAUCAGUACCUGG | | [2107-2127] 3′UTR |
| 366 | CUGUACUGAGACAUUACAGUU | AACUGUAAUGUCUCAGUACAG | | [1913-1933] 3′UTR |
| 367 | GUCUAGGAUGUUUCACAAACU | AGUUUGUGAAACAUCCUAGAC | | [1828-1848] ORF + 3′UTR |
| 368 | GCACUCUUCAGUUAUGAGGCU | AGCCUCAUAACUGAAGAGUGC | | [1642-1662] ORF |
| 369 | UCUACUCAUCUGGCCCAGAAA | UUUCUGGGCCAGAUGAGUAGA | | [125-145] 5′UTR |
| 370 | UUCCAGAUGAACCCAAGGAAA | UUUCCUUGGGUUCAUCUGGAA | | [1556-1576] ORF |
| 371 | CUCUGUGGUGUGAGAACACAG | CUGUGUUCUCACACCACAGAG | | [1520-1540] ORF |
| 372 | CCUGACUCUGUGGUGUGAGAA | UUCUCACACCACAGAGUCAGG | | [1515-1535] ORF |
| 373 | CCUUUCAGAAGACAGCAUGAA | UUCAUGCUGUCUUCUGAAAGG | | [1464-1484] ORF |
| 374 | GAACACACUAAGCUGAGCUAU | AUAGCUCAGCUUAGUGUGUUC | | [1414-1434] ORF |
| 375 | GGGUUUGUGCCUGAGACAAAA | UUUUGUCUCAGGCACAAACCC | | [1000-1020] ORF |
| 376 | CUGAUGCAUUUGCAACACUGA | UCAGUGUUGCAAAUGCAUCAG | | [74-94] 5′UTR |
| 377 | GAGGCUCACCGUGUGCUAUUU | AAAUAGCACACGGUGAGCCUC | | [979-999] ORF |
| 378 | GCCAAGAAGGAUUACCUAGGC | GCCUAGGUAAUCCUUCUUGGC | | [832-852] ORF |
| 379 | GCCCAGACAUUCCAAAAUCGA | UCGAUUUUGGAAUGUCUGGGC | | [711-731] ORF |
| 380 | UGAGCCCAGACAUUCCAAAAU | AUUUUGGAAUGUCUGGGCUCA | | [708-728] ORF |
| 381 | AGCAUUGGCCACGAGCAUGAA | UUCAUGCUCGUGGCCAAUGCU | | [684-704] ORF |
| 382 | GCUUUCAUGUAUGCCAAGAAG | CUUCUUGGCAUACAUGAAAGC | | [634-654] ORF |
| 383 | GGUUAUGAGUCAGUUGCCAAA | UUUGGCAACUGACUCAUAACC | | [38-58] 5′UTR |
| 384 | UGUUUGCCUGUGAGGUGUUAU | AUAACACCUCACAGGCAAACA | | [605-625] ORF |
| 385 | GAAGCCUUGAUUCAGCUUCGA | UCGAAGCUGAAUCAAGGCUUC | | [538-558] ORF |
| 386 | GAGAAAUAUGAUUUGGCUAUC | GAUAGCCAAAUCAUAUUUCUC | | [505-525] ORF |
| 387 | CUCUACUACCAGACAGAGAAA | UUUCUCUGUCUGGUAGUAGAG | | [490-510] ORF |
| 388 | GGCUUACUUCCAACGAGGGAU | AUCCCUCGUUGGAAGUAAGCC | Guinea-pig | [468-488] ORF |
| 389 | CAAGCACUUGGCAGUGGCUUA | UAAGCCACUGCCAAGUGCUUG | Rat | [453-473] ORF |
| 390 | AGAGAAGGCCUUUACCAGAAG | CUUCUGGUAAAGGCCUUCUCU | | [420-440] ORF |
| 391 | UUGCUUCAACAUUGGCUGCAU | AUGCAGCCAAUGUUGAAGCAA | | [369-389] ORF |
| 392 | AGGCUUGAAGUAACCUUAUAG | CUAUAAGGUUACUUCAAGCCU | | [2321-2341] 3′UTR |
| 393 | CCAUUACUGUAAUUGGCUCUU | AAGAGCCAAUUACAGUAAUGG | | [2299-2319] 3′UTR |
| 394 | GCAUCUUACAAAAACAAGACA | UGUCUUGUUUUUGUAAGAUGC | | [2231-2251] 3′UTR |
| 395 | CACUUUUCUGUGUCAUACCAC | GUGGUAUGACACAGAAAAGUG | | [2194-2214] 3′UTR |
| 396 | GUUCACACUGUAAACAUUCAU | AUGAAUGUUUACAGUGUGAAC | | [2155-2175] 3′UTR |
| 397 | AGCUGUGUUCACACUGUAAAC | GUUUACAGUGUGAACACAGCU | | [2149-2169] 3′UTR |
| 398 | UGGCCUCCUAGUUUCUACCUA | UAGGUAGAAACUAGGAGGCCA | | [229-249] 5′UTR |
| 399 | UGUUUCUUGCUCUGAGUGCUA | UAGCACUCAGAGCAAGAAACA | | [2121-2141] 3′UTR |
| 400 | UUCCAGGUACUGAUCCUGUUU | AAACAGGAUCAGUACCUGGAA | | [2105-2125] 3′UTR |
| 401 | AGAGUCUUCCAGGUACUGAUC | GAUCAGUACCUGGAAGACUCU | | [2099-2119] 3′UTR |
| 402 | CUGAGACAUUACAGUUUGGAA | UUCCAAACUGUAAUGUCUCAG | | [1918-1938] 3′UTR |
| 403 | GCCCUAUUACUUGUUUGUAAG | CUUACAAACAAGUAAUAGGGC | | [1869-1889] 3′UTR |
| 404 | UCUAGGAUGUUUCACAAACUA | UAGUUUGUGAAACAUCCUAGA | | [1829-1849] ORF + 3′UTR |
| 405 | GGAUAUAAUCCUGGUGUUAUC | GAUAACACCAGGAUUAUAUCC | | [1695-1715] ORF |
| 406 | GGGAUAUAAUCCUGGUGUUAU | AUAACACCAGGAUUAUAUCCC | | [1694-1714] ORF |
| 407 | GAGGCACUCUUCAGUUAUGAG | CUCAUAACUGAAGAGUGCCUC | | [1639-1659] ORF |
| 408 | AAAGUGAAAAAGCUGAUGCUA | UAGCAUCAGCUUUUUCACUUU | | [1574-1594] ORF |
| 409 | GAAAGUGAAAAAGCUGAUGCU | AGCAUCAGCUUUUUCACUUUC | | [1573-1593] ORF |
| 410 | GGAAAGUGAAAAAGCUGAUGC | GCAUCAGCUUUUUCACUUUCC | | [1572-1592] ORF |
| 411 | ACCAAGGCUUUCCAGAUGAAC | GUUCAUCUGGAAAGCCUUGGU | | [1547-1567] ORF |
| 412 | GGUGACCAAGGCUUUCCAGAU | AUCUGGAAAGCCUUGGUCACC | | [1543-1563] ORF |
| 413 | CAGCUCCUCCUAGUUCCAAAG | CUUUGGAACUAGGAGGAGCUG | | [1205-1225] ORF |
| 414 | UGCAACACUGAGAAGUUAUCU | AGAUAACUUCUCAGUGUUGCA | | [84-104] 5′UTR |
| 415 | UGCAUUUGCAACACUGAGAAG | CUUCUCAGUGUUGCAAAUGCA | | [78-98] 5′UTR |
| 416 | AUGCAUUUGCAACACUGAGAA | UUCUCAGUGUUGCAAAUGCAU | | [77-97] 5′UTR |
| 417 | CCUGAGACAAAAGAAGAGCUC | GAGCUCUUCUUUUGUCUCAGG | | [1009-1029] ORF |
| 418 | AGACAGUUUCUCUGGGUUUGC | GCAAACCCAGAGAAACUGUCU | | [885-905] ORF |
| 419 | CCAAAUGAGAGACAAGUGGCU | AGCCACUUGUCUCUCAUUUGG | | [805-825] ORF |
| 420 | CCCAGACAUUCCAAAAUCGAC | GUCGAUUUUGGAAUGUCUGGG | | [712-732] ORF |
| 421 | AGCUGAAGAACAGUUAGCAUU | AAUGCUAACUGUUCUUCAGCU | | [669-689] ORF |
| 422 | GAAAAAAGCUGAAGAACAGUU | AACUGUUCUUCAGCUUUUUUC | | [663-683] ORF |
| 423 | GGAAAAAAGCUGAAGAACAGU | ACUGUUCUUCAGCUUUUUUCC | | [662-682] ORF |
| 424 | UACCAGACAGAGAAAUAUGAU | AUCAUAUUUCUCUGUCUGGUA | | [496-516] ORF |
| 425 | UGGCAGUGGCUUACUUCCAAC | GUUGGAAGUAAGCCACUGCCA | | [461-481] ORF |
| 426 | GAAGGCCUUUACCAGAAGCAU | AUGCUUCUGGUAAAGGCCUUC | | [423-443] ORF |
| 427 | UCCCGGAUUUGCUUCAACAUU | AAUGUUGAAGCAAAUCCGGGA | | [361-381] ORF |
| 428 | CCAUCAGCCUCUGGAAUGAAG | CUUCAUUCCAGAGGCUGAUGG | | [269-289] ORF |
| 429 | CCUAAUCAUGUCCCUGGUGGA | UCCACCAGGGACAUGAUUAGG | | [246-266] 5′UTR + ORF |
| 430 | CUACCUAAUCAUGUCCCUGGU | ACCAGGGACAUGAUUAGGUAG | | [243-263] 5′UTR + ORF |
| 431 | GGAGUAAGUACAAAUAUUCCU | AGGAAUAUUUGUACUUACUCC | | [2269-2289] 3′UTR |
| 432 | CAAAAACAAGACACCCAAGUC | GACUUGGGUGUCUUGUUUUUG | | [2239-2259] 3′UTR |
| 433 | ACAGUUUGGAAGUGUUAACUA | UAGUUAACACUUCCAAACUGU | | [1928-1948] 3′UTR |
| 434 | CUGUCUUCUCUCUGUCUCCUG | CAGGAGACAGAGAGAAGACAG | | [196-218] 5′UTR |
| 435 | CUAGGAUGUUUCACAAACUAC | GUAGUUUGUGAAACAUCCUAG | | [1830-1850] ORF + 3′UTR |
| 436 | GGCACUCUUCAGUUAUGAGGC | GCCUCAUAACUGAAGAGUGCC | | [1641-1661] ORF |
| 437 | CAAGGUGCACUACAAGUACAC | GUGUACUUGUAGUGCACCUUG | Guinea-pig | [1314-1334] ORF |
| 438 | UGAUAACUGGGCCACGGUCAU | AUGACCGUGGCCCAGUUAUCA | | [1074-1094] ORF |
| 439 | GUGGAUCAAGACAGUUUCUCU | AGAGAAACUGUCUUGAUCCAC | | [877-897] ORF |
| 440 | ACAUUUCCUGAUGCAUUUGCA | UGCAAAUGCAUCAGGAAAUGU | | [67-87] 5′UTR |
| 441 | AGACAUUCCAAAAUCGACAAG | CUUGUCGAUUUUGGAAUGUCU | | [715-735] ORF |
| 442 | GCUGAAGAACAGUUAGCAUUG | CAAUGCUAACUGUUCUUCAGC | | [670-890] ORF |
| 443 | AAGCUGAAGAACAGUUAGCAU | AUGCUAACUGUUCUUCAGCUU | | [668-688] ORF |
| 444 | CUGUUUGCCUGUGAGGUGUUA | UAACACCUCACAGGCAAACAG | | [604-624] ORF |
| 445 | GUUCAAGCUGUUUGCCUGUGA | UCACAGGCAAACAGCUUGAAC | Mouse | [597-617] ORF |
| 446 | CUGAUAGACUAUAAGAUCCUG | CAGGAUCUUAUAGUCUAUCAG | Guinea-pig | [568-588] ORF |
| 447 | GCUGAUAGACUAUAAGAUCCU | AGGAUCUUAUAGUCUAUCAGC | | [567-587] ORF |
| 448 | GGCUAUCAAAGACCUUAAAGA | UCUUUAAGGUCUUUGAUAGCC | | [519-539] ORF |
| 449 | GGGAUGCUCUACUACCAGACA | UGUCUGGUAGUAGAGCAUCCC | | [484-504] ORF |
| 450 | CACUUGGCAGUGGCUUACUUC | GAAGUAAGCCACUGCCAAGUG | Rat | [457-477] ORF |
| 451 | CCUUAUAGGUUACUCAUAAGG | CCUUAUGAGUAACCUAUAAGG | | [2334-2354] 3′UTR |
| 452 | UGAAGUAACCUUAUAGGUUAC | GUAACCUAUAAGGUUACUUCA | | [2326-2346] 3′UTR |
| 453 | UAAGGCUUGAAGUAACCUUAU | AUAAGGUUACUUCAAGCCUUA | | [2319-2339] 3′UTR |
| 454 | CAAAUAUUCCUGUUUCUGAAC | GUUCAGAAACAGGAAUAUUUG | | [2279-2299] 3′UTR |
| 455 | CUGUGUUCACACUGUAAACAU | AUGUUUACAGUGUGAACACAG | | [2151-2171] 3′UTR |
| 456 | CAGGUACUGAUCCUGUUUCUU | AAGAAACAGGAUCAGUACCUG | | [2108-2128] 3′UTR |
| 457 | UUAGCACCCUUCUGCUGUAUA | UAUACAGCAGAAGGGUGCUAA | | [1892-1912] 3′UTR |
| 458 | UGAAGCCCUAUUACUUGUUUG | CAAACAAGUAAUAGGGCUUCA | | [1865-1885] 3′UTR |
| 459 | GAAAAUGAAGCCCUAUUACUU | AAGUAAUAGGGCUUCAUUUUC | | [1860-1880] 3′UTR |
| 460 | UUCACAAACUACAAAGCUGAA | UUCAGCUUUGUAGUUUGUGAA | | [1839-1859] 3′UTR |
| 461 | ACUCGGAGAGAAGUCUAGGAU | AUCCUAGACUUCUCUCCGAGU | | [1816-1836] ORF + 3′UTR |
| 462 | GCCCAGAAAGUGAACACCUUG | CAAGGUGUUCACUUUCUGGGC | | [137-157] 5′UTR |
| 463 | UUCAGUUAUGAGGCUACCCAA | UUGGGUAGCCUCAUAACUGAA | | [1648-1668] ORF |
| 464 | GUGCACUACAAGUACACGGUA | UACCGUGUACUUGUAGUGCAC | | [1318-1338] ORF |
| 465 | CCUAAGGAAGUGAAGCUCAGU | ACUGAGCUUCACUUCCUUAGG | | [1273-1293] ORF |
| 466 | GCCUAAGGAAGUGAAGCUCAG | CUGAGCUUCACUUCCUUAGGC | | [1272-1292] ORF |
| 467 | AGCCUAAGGAAGUGAAGCUCA | UGAGCUUCACUUCCUUAGGCU | | [1271-1291] ORF |
| 468 | CACCGUGUGCUAUUUGGGUUU | AAACCCAAAUAGCACACGGUG | | [985-1005] ORF |
| 469 | CUGUGGUGGAUCAAGACAGUU | AACUGUCUUGAUCCACCACAG | | [872-892] ORF |
| 470 | UGGGCAAGCUGUUUCGACCAA | UUGGUCGAAACAGCUUGCCCA | | [788-808] ORF |
| 471 | GUGUGUCUGGAAGCAGAAGCU | AGCUUCUGCUUCCAGACACAC | | [744-764] ORF |
| 472 | UCCAAAAUCGACAAGGCGAUG | CAUCGCCUUGUCGAUUUUGGA | | [721-741] ORF |
| 473 | CUGAGCCCAGACAUUCCAAAA | UUUUGGAAUGUCUGGGCUCAG | | [707-727] ORF |
| 474 | CAGCUGAUAGACUAUAAGAUC | GAUCUUAUAGUCUAUCAGCUG | | [565-585] ORF |
| 475 | AGGGAACCAGCUGAUAGACUA | UAGUCUAUCAGCUGGUUCCCU | | [558-578] ORF |
| 476 | UCAAAGACCUUAAAGAAGCCU | AGGCUUCUUUAAGGUCUUUGA | | [524-544] ORF |
| 477 | AGCACUUGGCAGUGGCUUACU | AGUAAGCCACUGCCAAGUGCU | Rat | [455-475] ORF |
| 478 | UGAAGCAGAGAAGGCCUUUAC | GUAAAGGCCUUCUCUGCUUCA | | [414-434] ORF |
| 479 | ACUGAAGCAGAGAAGGCCUUU | AAAGGCCUUCUCUGCUUCAGU | | [412-432] ORF |
| 480 | CUCCCGGAUUUGCUUCAACAU | AUGUUGAAGCAAAUCCGGGAG | | [360-380] ORF |
| 481 | GAAGUAACCUUAUAGGUUACU | AGUAACCUAUAAGGUUACUUC | | [2327-2347] 3′UTR |
| 482 | GGCUUGAAGUAACCUUAUAGG | CCUAUAAGGUUACUUCAAGCC | | [2322-2342] 3′UTR |
| 483 | UUAAGGCUUGAAGUAACCUUA | UAAGGUUACUUCAAGCCUUAA | | [2318-2338] 3′UTR |
| 484 | UGCACUUUUCUGUGUCAUACC | GGUAUGACACAGAAAAGUGCA | | [2192-2212] 3′UTR |
| 485 | CAAGCUGUACAUUUGGUGCAC | GUGCACCAAAUGUACAGCUUG | | [2176-2196] 3′UTR |
| 486 | GUGUUCACACUGUAAACAUUC | GAAUGUUUACAGUGUGAACAC | | [2153-2173] 3′UTR |
| 487 | AGCUCUCUUGGCCUCCUAGUU | AACUAGGAGGCCAAGAGAGCU | | [221-241] 5′UTR |
| 488 | AUGAAGCCCUAUUACUUGUUU | AAACAAGUAAUAGGGCUUCAU | | [1864-1884] 3′UTR |
| 489 | GGAAAGCACUCGGAGAGAAGU | ACUUCUCUCCGAGUGCUUUCC | | [1809-1829] ORF |
| 490 | CUGCGCAACUACAGAUUUGGA | UCCAAAUCUGUAGUUGCGCAG | | [1791-1811] ORF |
| 491 | GGCCCAGAAAGUGAACACCUU | AAGGUGUUCACUUUCUGGGCC | | [138-156] 5′UTR |
| 492 | CUCAAGGUGCACUACAAGUAC | GUACUUGUAGUGCACCUUGAG | Guinea-pig | [1312-1332] ORF |
| 493 | AGUGAAGCUCAGUGUUCCCAU | AUGGGAACACUGAGCUUCACU | | [1281-1301] ORF |
| 494 | UGCAACUACCUUGAACCAGUU | AACUGGUUCAAGGUAGUUGCA | Guinea-pig | [1123-1143] ORF |
| 495 | GCUUGUUCCCUGCAACUACCU | AGGUAGUUGCAGGGAACAAGC | Guinea-pig | [1113-1133] ORF |
| 496 | AGGCUCACCGUGUGCUAUUUG | CAAAUAGCACACGGUGAGCCU | | [980-1000] ORF |
| 497 | GCUGGCCAAGAAGGAUUACCU | AGGUAAUCCUUCUUGGCCAGC | | [828-848] ORF |
| 498 | CAUUUCCUGAUGCAUUUGCAA | UUGCAAAUGCAUCAGGAAAUG | | [68-88] 5′UTR |
| 499 | CAGAAGCUAUAUGAGCCAGUG | CACUGGCUCAUAUAGCUUCUG | | [757-777] ORF |
| 500 | GAGCCCAGACAUUCCAAAAUC | GAUUUUGGAAUGUCUGGGCUC | | [709-729] ORF |
| 501 | GCUCUACUACCAGACAGAGAA | UUCUCUGUCUGGUAGUAGAGC | | [489-509] ORF |
| 502 | ACCAGAAGCAUUAACCGAGAC | GUCUCGGUUAAUGCUUCUGGU | | [433-453] ORF |
| 503 | GCUUGAAGUAACCUUAUAGGU | ACCUAUAAGGUUACUUCAAGC | | [2323-2343] 3′UTR |
| 504 | UCUUGCUCUGAGUGCUAGCUA | UAGCUAGCACUCAGAGCAAGA | | [2125-2145] 3′UTR |
| 505 | CCAGAAAGUGAACACCUUGGG | CCCAAGGUGUUCACUUUCUGG | | [139-159] 5′UTR |
| 506 | UCCUGGUGUUAUCAAAGGUGA | UCACCUUUGAUAACACCAGGA | | [1703-1723] ORF |
| 507 | CAACCAGAGGACCUGGAGUUU | AAACUCCAGGUCCUCUGGUUG | | [1666-1686] ORF |
| 508 | GGUGCACUACAAGUACACGGU | ACCGUGUACUUGUAGUGCACC | | [1317-1337] ORF |
| 509 | GCAACUACCUUGAACCAGUUG | CAACUGGUUCAAGGUAGUUGC | Guinea-pig | [1124-1144] ORF |
| 510 | GUCUGAGCCCAGACAUUCCAA | UUGGAAUGUCUGGGCUCAGAC | | [705-725] ORF |
| 511 | CCAGUUCAAGCUGUUUGCCUG | CAGGCAAACAGCUUGAACUGG | | [594-614] ORF |
| 512 | GGAUGCUCUACUACCAGACAG | CUGUCUGGUAGUAGAGCAUCC | | [485-505] ORF |
| 513 | AGGGAUGCUCUACUACCAGAC | GUCUGGUAGUAGAGCAUCCCU | | [483-503] ORF |
| 514 | GCCUUUACCAGAAGCAUUAAC | GUUAAUGCUUCUGGUAAAGGC | | [427-447] ORF |
| 515 | AGGCCUUUACCAGAAGCAUUA | UAAUGCUUCUGGUAAAGGCCU | | [425-445] ORF |
| 516 | GAACCAUUACUGUAAUUGGCU | AGCCAAUUACAGUAAUGGUUC | | [2296-2316] 3′UTR |
| 517 | AGCAUCUUACAAAAACAAGAC | GUCUUGUUUUUGUAAGAUGCU | | [2230-2250] 3′UTR |
| 518 | CUAGCUAGCCAGCUGUGUUCA | UGAACACAGCUGGCUAGCUAG | | [2139-2159] 3′UTR |
| 519 | CCAUUCUACUCAUCUGGCCCA | UGGGCCAGAUGAGUAGAAUGG | | [121-141] 5′UTR |
| 520 | UCAGCUGGCCAAGAAGGAUUA | UAAUCCUUCUUGGCCAGCUGA | | [825-845] ORF |
| 521 | CAGUUCAAGCUGUUUGCCUGU | ACAGGCAAACAGCUUGAACUG | Mouse | [595-615] ORF |
| 522 | CUCUUAAGGCUUGAAGUAACC | GGUUACUUCAAGCCUUAAGAG | | [2315-2335] 3′UTR |
| 523 | ACCAUUACUGUAAUUGGCUCU | AGAGCCAAUUACAGUAAUGGU | | [2298-2318] 3′UTR |
| 524 | CCUGUUUCUGAACCAUUACUG | CAGUAAUGGUUCAGAAACAGG | | [2287-2307] 3′UTR |
| 525 | UCCAGGCCCAAGGAGUAAGUA | UACUUACUCCUUGGGCCUGGA | | [2258-2278] 3′UTR |
| 526 | GAGAGUCUUCCAGGUACUGAU | AUCAGUACCUGGAAGACUCUC | | [2098-2118] 3′UTR |
| 527 | AGCCCUAUUACUUGUUUGUAA | UUACAAACAAGUAAUAGGGCU | | [1868-1888] 3′UTR |
| 528 | GAAGCCCUAUUACUUGUUUGU | ACAAACAAGUAAUAGGGCUUC | | [1866-1886] 3′UTR |
| 529 | CACUACAAGUACACGGUAGUC | GACUACCGUGUACUUGUAGUG | | [1321-1341] ORF |
| 530 | AGGGCAAUGAUAACUGGGCCA | UGGCCCAGUUAUCAUUGCCCU | | [1067-1087] ORF |
| 531 | UCACCGUGUGCUAUUUGGGUU | AACCCAAAUAGCACACGGUGA | | [984-1004] ORF |
| 532 | UUGGCUAUCAAAGACCUUAAA | UUUAAGGUCUUUGAUAGCCAA | | [517-537] ORF |
| 533 | GCACCCUUCUGCUGUAUACUG | CAGUAUACAGCAGAAGGGUGC | | [1895-1915] 3′UTR |
| 534 | AGCACCCUUCUGCUGUAUACU | AGUAUACAGCAGAAGGGUGCU | | [1894-1914] 3′UTR |
| 535 | UGGAAAAAAGCUGAAGAACAG | CUGUUCUUCAGCUUUUUUCCA | | [661-681] ORF |
| 536 | GGCUCCAGUUCAAGCUGUUUG | CAAACAGCUUGAACUGGAGCC | | [590-610] ORF |
| 537 | GAUUUGGCUAUCAAAGACCUU | AAGGUCUUUGAUAGCCAAAUC | | [514-534] ORF |
| 538 | UGACUGAAGCAGAGAAGGCCU | AGGCCUUCUCUGCUUCAGUCA | | [410-430] ORF |
| 539 | CUGUUUCUGAACCAUUACUGU | ACAGUAAUGGUUCAGAAACAG | | [2288-2308] 3′UTR |
| 540 | GACUCUGUGGUGUGAGAACAC | GUGUUCUCACACCACAGAGUC | | [1518-1538] ORF |
| 541 | GCUGUUUGCCUGUGAGGUGUU | AACACCUCACAGGCAAACAGC | | [603-623] ORF |
| 542 | CUCUCAUGGUAGGGUUAUGAG | CUCAUAACCCUACCAUGAGAG | | [26-46] 5′UTR |
| 543 | ACAUUGGCUGCAUGUACACUA | UAGUGUACAUGCAGCCAAUGU | | [377-397] ORF |
| 544 | AAGCCCUAUUACUUGUUUGUA | UACAAACAAGUAAUAGGGCUU | | [1857-1887] 3′UTR |
| 545 | GCCAAGAAGGAGGAAUGGAAA | UUUCCAUUCCUCCUUCUUGGC | Guinea-pig | [646-666] ORF |
| 546 | AGGUCAUGCCAGGGAACAUUG | CAAUGUUCCCUGGCAUGACCU | | [1031-1051] ORF |
| 547 | UGCUAGCUAGCCAGCUGUGUU | AACACAGCUGGCUAGCUAGCA | | [2137-2157] 3′UTR |
| 548 | CAGCUUAAGAAAGGCAGCCAA | UUGGCUGCCUUUCUUAAGCUG | | [1615-1635] ORF |
| 549 | UGCCAAGAAGGAGGAAUGGAA | UUCCAUUCCUCCUUCUUGGCA | | [645-665] ORF |
| 550 | UGGCUAUCAAAGACCUUAAAG | CUUUAAGGUCUUUGAUAGCCA | | [518-538] ORF |
| 551 | UGAUUUGGCUAUCAAAGACCU | AGGUCUUUGAUAGCCAAAUCA | | [513-533] ORF |
| 552 | AGAAGGCCUUUACCAGAAGCA | UGCUUCUGGUAAAGGCCUUCU | | [422-442] ORF |
| 553 | CAGAGAAGGCCUUUACCAGAA | UUCUGGUAAAGGCCUUCUCUG | | [419-439] ORF |
| 554 | GCAGAGAAGGCCUUUACCAGA | UCUGGUAAAGGCCUUCUCUGC | | [418-438] ORF |
| 555 | AUAUUCCUGUUUCUGAACCAU | AUGGUUCAGAAACAGGAAUAU | | [2282-2302] 3′UTR |
| 556 | AGCUUAAGAAAGGCAGCCAAG | CUUGGCUGCCUUUCUUAAGCU | | [1616-1636] ORF |
| 557 | UCAGCUUAAGAAAGGCAGCCA | UGGCUGCCUUUCUUAAGCUGA | | [1614-1634] ORF |
| 558 | CAGGUCAUGCCAGGGAACAUU | AAUGUUCCCUGGCAUGACCUG | | [1030-1050] ORF |
| 559 | CUAGCAUCUUACAAAAACAAG | CUUGUUUUUGUAAGAUGCUAG | | [2228-2248] 3′UTR |

1. A double-stranded siRNA compound having structure (A) set forth below:
5′(N)ₓ—Z3′ (antisense strand)
3′Z′—(N′)_{y}5′ (sense strand) (A)
wherein each of N and N′ is a nucleotide selected from an unmodified ribonucleotide, a modified ribonucleotide, an unmodified deoxyribonucleotide and a modified deoxyribonucleotide;
wherein each of N and N′ is a nucleotide which may be modified or unmodified in its sugar residue;
wherein each of (N)ₓ and (N′)_{y} is an oligonucleotide in which each consecutive N or N′ is joined to the next N or N′ by a covalent bond;
wherein each of x and y is an integer between 18 and 40;
wherein each of Z and Z′ may be present or absent, but if present is 1-5 consecutive nucleotides covalently attached at the 3′ terminus of the strand in which it is present; and
wherein the sequence of (N)ₓ comprises an antisense sequence to an mRNA transcribed from mammalian gene selected from the group consisting of NOX4, NOX1, NOX2 (gp91phox, CYBB), NOX5, DUOX2, NOXO1, NOXO2, NOXA1 and NOXA2 (p67phox).

2. The compound of claim 1, wherein the sequence of (N)ₓ comprises an antisense sequence set forth in any one of SEQ ID NOs: 13-17, 352.

3. The compound of claim 1, wherein the covalent bond is a phosphodiester bond.

4. The compound of claim 1, wherein x=y.

5. (canceled)

6. The compound of claim 4, wherein x=y=19.

7. The compound of claim 1, wherein Z and Z′ are both absent.

8. The compound of claim 1, wherein one of Z or Z′ is present.

9. The compound of claim 1, wherein all of the ribonucleotides are unmodified in their sugar residues.

10. The compound of claim 1, wherein at least one ribonucleotide is modified in its sugar residue.

11. The compound of claim 10, wherein the modification of the sugar residue comprises a modification at the 2′ position.

12. The compound of claim 11, wherein the modification at the 2′ position results in the presence of a moiety selected from the group consisting of an amino, a fluoro, a methoxy, an alkoxy and an alkyl group.

13. The compound of claim 12, wherein the moiety at the 2′ position is methoxy (2′-O-methyl).

14. The compound of claim 1, wherein alternating ribonucleotides are modified in both the antisense and the sense strands.

15. The compound according to claim 14 wherein the middle ribonucleotide in the antisense strand is unmodified.

16. The compound of claim 14, wherein the ribonucleotides at the 5′ and 3′ termini of the antisense strand are modified in their sugar residues, and the ribonucleotides at the 5′ and 3′ termini of the sense strand are unmodified in their sugar residues.

17. The compound of claim 14, wherein the antisense and the sense strands are non-phosphorylated at the 3′ and 5′ termini or wherein the antisense and the sense strands are phosphorylated at the 3′ termini.

18. The compound of claim 1, wherein at least one modified ribonucleotide is selected from a locked ribonucleotide, a morpholino, a peptide ribonucleotide and a mirror ribonucleotide.

19-20. (canceled)

21. A pharmaceutical composition comprising one or more compound of claim 1 in an amount effective to inhibit gene expression of a gene selected from the group consisting of NOX4, NOX1, NOX2 (gp91phox, CYBB), NOX5, DUOX2, NOXO1, NOXO2, NOXA1 and NOXA2 (p67phox); and a pharmaceutically acceptable carrier.

22. A method of treating a subject in need of treatment for a disease or condition selected from hearing loss, acute renal failure, nephritis, ocular disease, Acute Respiratory Distress Syndrome and other acute lung injuries, lung transplantation, spinal cord injury, pressure sores, osteoarthritis and Chronic Obstructive Pulmonary Disease (COPD), comprising administering to the subject a compound according to claim 1 in an amount effective to treat the disease or condition.

23-28. (canceled)

29. The method of claim 22 wherein the disease or disorder is selected from COPD, Acute Respiratory Distress Syndrome or acute lung injury.

30. (canceled)

31. A method of treating an organ recipient comprising the step of administering to the organ recipient a therapeutically effective amount of a compound according to claim 1.

32. The method of claim 31 wherein the organ recipient is undergoing lung transplantation.
